SEQUENCE LISTING (34 sequences)

SEQ ID NO: 1 - DNA, 82393 bp, Homo sapiens
  source 1..82393: mol_type = unassigned DNA
acaacgcgctccctgcggggcgggcggcaacctccatgcggcctcgtccacgctcagcaccggggaagccgaggcggagaagccgcgcgcgcctcagaagctcccggacgcccaggtacgtgggagcactccacagatggcgcaagtacggttgggccaggcggttgcggccccgtcgcgccgcggcctctgtgacgcacggcgaggcctcccgggctgctgcgcggcgcagcgggggcggggcgagcgcgtgagggcggggcggtggggggggggggcggagcgagaggggcggagccggcagaggccccgccccggggccggaggagcgaggacgctacggagcaggcgcgtctcgctgccgccgctgccgccgccgccgctcgctcttctgtggagccgccgccgccgccgccgccatttgcacggggaccccagtgacaggggctcggcggaggggcggaggggcggagggagggggggagggcccgcggagcccccgagggcgggagcgacgccgccggcgccggccgggctccctgcgcgaccgcgccgcccgcggcgggccccgagcagcagcagcagcagcagcggcagcagcggcagcagcagcagcagccgaggccgggcgtgcgcctgaggcggcggcggcggcggccctgcgggcggccgggaggggcgggggcagcggccgccgccgtttgatggatccgaggatcgcctggtttcagccagagcagctcggaccgtccaacagtctgtggatgcagatctgggagacgacccaggggctgaggaacctctacttcaaccaccactgtcacagcagcggcggcgcgagcggcggcggcggcagcagcagcagcagcagcacggccaccggcgggagcggcagcagcaccggcagccccggcggcgcggcctcggccccggccccggccccggccggcatgtatcgctccggggagcgcctgctgggcagccacgcgctgcccgcggagcagcgggacttcctgcccctagagacgaccaacaacaacaacaaccaccaccagcccggggcctgggcccgccgggcgggctcctcggcgtcctcgcctccctcggcgtcctcgtccccgcacccttcggccgccgtccccgccgccgatccagccgattcggcctcgggcagcagcaacaagaggaagcgcgacaacaaggccagcacgtatggactcaactacagcctgctgcagcccagcggagggcgggccgcggggggcggccgagcagacggcggcggggtcgtgtacagcgggaccccgtggaaacggaggaactacaaccagggagtcgtggggtgagtgctggctctgcggcccgatggcctggccggtgcgaatgcgcagccgggcacacgcccacagaggggggttgtgagggtctaggagcggccacccccacggcctgccttcgctgctgttgcacgggggtgctgctggccatccccaaccccccagtcgttcacacctttccccaagcctccttagccgtccacaccctccgtctcctgtcctcccttagtcgtccacaccttcctcccctccctcttaaccgtccacaccttccccaggcccccccctttatccattcactctcctcccatcccccttagttaaacacatctacccttgaccaccaccccgcctccagccctccacacctttttccccatcatcacaactcaagatgagaccgcttagcacgggcctatcattcattccctgagaacattggtgtgtgagtgttttttgatggtgcaggacccggaggtgctttccttgccaagaatagaaacatccagaatgctcctccccatcccccaatcccagacagcaattatgtcagccctgtaaggcattgcctgctcttgaccctttggcccatctttttatttttaaaaaattcccatgtcacagatgccctgtctatgcagagggtggcgtgggatgggtgaccactaagtttaggctggtgaaggtggtgagcccttctgaggccctgatagaactttccaggagttcatggtccgcggctccagcttctcactgtaaagttgtcatcctggcagaggcagccaatgcttttcattctagggggtagagatttatgctaatgagtgaatattgcaccactagtgactttctgtttaaagttcagctcttagaaaatggaatcttacctgacccctagtgaattatgtacataagcagggaatgtttccaactagatctcccttcagaagagtccctgtgctggaataggtcactgaatcttatttgttttgtaaaacaaagcttttgggtctcgtgggtgtgtgtgtgtgtgtgtgtgtgtgtgtgtgtgtgtgtgtagcttgagtatggagaaccggctttcaaattgcttttcatttttcaggttgtgttttacattgagggctttagcatgcaaatgaaattaccaattagtaatcccatgtgaaccttttcctggatttattcattcagatctgccctgctttggctgagagagagagttctgtgtacctttttgaaggtctggataaaatgagttggtgggttccatctgcttccagtgggctggtgtctgctctatgctactattacaactcctaccttttgtggaaaatgcagtcaagcgttctaggactggtgctgtggtacatgtcaaacctgccctcacattccagaaagggaacccttttagggttgagtcctctgttgctaagcttcaagggtgctctccatggtcatcacgttttattaaaggcttgtggttccatcctgttagcatttccaagtctacgcgtaaacctgtggtttagtgacaagcaaattgatgttgagggtttctggtagtttcatttcacaggagtaagctccagttaggtaatcactgtcaacgaaaaccttgaagttccttaattgcattttactgaagcctctttgcatgtgtctagcaaaagatataagtccaagatgcttattttttttttgataaattagaaattgtcctctcctctacttgctatttaatgcagaagatactctaaaaggttcatatttatacttagaagcaagatgttcttgttcctgattcaaatatattgccctcaaagggattaggagaggaattttcatttcccggagggattactgtttaaaaactggttgtaaacctctttaaaaactgcttatcacttcaccagattttccattcttttgcctcctcccttagaggatgtcagcagttaattttttttttaaattaaaaaaagttcaattctgagacctcctagtttcaaaaaatacattaaacaattcccaagagtgttaagagtgtctgggtgcttagaaattcttgctttgattcatgtattctgattttttttttttttttgagacggagtttcgctcttgttgcccaggctggagtgcagtggctcgatctcagctcaccgcaacctctgcctcccaggttcaagcgattctgctgcctcagcctcccgagtagctgggattacaggcgcttgccaccacgcctggctaattttttatttttagtagagacggggtttcttcatgttggtcaggctggtctcgaactcctgacctcaggtgatctgcccgtcttggcctcccaaaggactgggattacaggcatgagccaccgtgcccggcctcattatcctgatttcttttttttcttttgagactgggtctcactctgctgcctaggctggagtggagtgacgtgatcatagctcactgtatcctctaactcttgggctcaagtgatcctcctgccttagcttcctggagtagctgggactacaggcacatgtcaccacacctgcctaatttttttatttttactttttgtagagatggggcctccatttgttgcctaggctggtcttcaaccggcctcaagcagtcctcccaccttggcctcacagagtgctgggattataggcatgagccaccattctcgccagtatccttatttcttaactttagaaagtttttctatttttaatataggtatttaaaaaaatctgaattcagagtgcacctcgatgttatgctgttctgagattaaatatactaaaactgttaccattgttttctgaattcttaagatgtgactgatagttagctaataggttaacacattgtggtggttcttggcctctgaactgatagtccagatggggagaggagaccagaaagcatgtgaaaatggactagaaccatgggacagctatatagtctctcgcagctgtcttttgtgttctctgcttccaccaaattggttgatttatttagaatgctgacctcttgcattgcctaagtccttgatgtttttggtttctcctctgaactctcaaaggtactcacttcatgctcttggtatagcccacttatgtttaactttccttttattatgtgttccctcttacacatgacatggacatttctttaatatgtagagtaagatattggatttcatcctaaagtcttcaaaataaaactcttgagctcatcatctcagacttcttcatgtacccacagaccagggattttgtttgctttttaaaacatttttttattttgtgttttattatttttaaattttaatttaattttatggagacagggtctcgctctgttgcccaggctggagtgcagtggtgtgatctcggctcactgcagcctttgcctgggctcaagccatccacatgccttggcctcccagtgtgctgggattacaggtgtgagccactgtgcctggcctaaatttatttttttaattttttttgagacagggtcttgctctgtcgctcaggctggagtgcagtgtcataatcatgggtcacagcagccttggcctcccaggctgaagtgaacttcccacctcagcctcctgagtagttgggactacaggcgagtgccaccatgcctggctcattttggttttttttgtaaagatggggtcttgtcatgttgcccatgaaggtctccaactcctggtccaagtgatcctcccgcctccgcctagcaaaatgttgggattacaggtgtgagccaccatgcctggccttatttatttatttaattatgaatgaatgaatgaattaatgagagggagtcttgctctcttgcccaggctggagtgtggtggcacaatcttggcccactgcaacctccgtctcccaggttcgagcaattctcctgcctcagcctcccgagtaactgggattacaggcgcccgccaccatgcccaggtaatttttgtatttttagtagagatggggtctcaccatgttggccagactggtttcgaacttctgacctcaagtgatctgcccaccttggcttcccaaagtgtcaggattacaggcatgagccaccatgcctggcctggccttttatgttttaagttgcttccactgattctcttgggctttgctcccctccagaactggccatggtttaggatgctgtccacctgctgctgcttgtccatgaaaacgagccataaacccttttcttttgaaagacttaattgtttatcactatggagaaagaggggatggcaagaagtagcaaatacagggaatttgcagaacttggtcttgagccctgggtccagaaacttcttctggaaggtgcttggtgtttgtccaagctcatgataggtttctgttggctgtactgccagatctgtagatgcttttttaaggcttggatgacttgttcaaaacaatgttttggagtacaaatttggctgtggggacatcaagaccttgttgggaaacttgggtttaaggtacaatttcttaaactaggatggtgggaatggggatgtgaagggagaatgaatgtgagaggtattacagggtaaggatggagatgattcagattccttaagtggatttaataatcacactgtagctttgaacttgagtgactggggaaatatttgtggtgtttttggaaataagggccagaaggactattggtttgggtaagaagatagtagggggatgtataggtggacctgctagtggggagctgagatttggagggctgagatgtagtgctcttcactgccgtagggcagtatcctcttgtatgtgccatcctctagtgcccattgttcatcatgtcatagtaagcccaagatgttcatgccttttttcagcactgcattagggcttatatctgcttctctttctctctctctctcgctctcgctccctccctctctctctctttctgttttttttttttttttagacggagtttcacttgtgttgcccaggttggagtgcagtggcgcgatcttggctcattgcaacctctgactcccgggttcaagcaattctcctgcctcagcctcccaagtagctgggattacaggcatgtgccaccatgcctaattttgtatctttagtagagatggggtttctccatgttggtcaggctggtgtcaaactcccaacatcaggtgatctacctgcctcggccgcccaaagtgctgggattacaggcatgagccaccgcgcccggcctctctctgcttatttctacacagtgttaccaatgagattggtgttactgctgggctccaaagcaatcagacagattaaagtagattgaatatgaaagaatttagaggcctttttccaagtgatttgtgctctatttaatttctgtgcatttgcagatatagcccacagtaattcttagtgaactagaaccttcaggttattgaattttactgatttgggtactgacatgcgcttttaagaagacattaggttttctatagtgtagattgtacactaacaatataattcatatttaagaatgtctcaaaatttagtatactgtgttcaactaacttaactttctttgttttttttgttttgttttgtttttgttttttgagacggagtcttgctatgccacccaggctggagtgcagtggcgtgatcttggcttactgcaacttcaacactcctgggttcaagtgattttcctgcctcagcctcctgagtagctgggattacaggcacccgccaccacaccggctaatttttgtatttttagtagagacggggtttcgccgtgttggccaggctggtcttggactcctgactcaaatgatctgcctgccttggcctcccacagtgctaggattacagacatgagccactgcgcccggccgctaacttaactttcattccacaacttccatcttttatccaaaatctgtgatcattgaatactgtcaccattaatcattggcatttcagtgtttggactttttttttcccccttcgtctttgtggactcttttttaacactcataaagttttaactattgaaaagcaaaggaaacggtgagtgactttttggagtctgtctacccagtggtcacacaaaaggcttactacattacaggaaagataggatgggaaagggatactagaaaattctaagtcaggaacgggggtgtgtattagaaaaattctgatcctggcatgccagatggccttacatctcaatttcttccgtgaaattcctgccaacaaatcatagtgttagaagtacagaagggtccatgggaacagaatttaagggctccgttggtgatacggaactgatcagatggttctcacttgttctcagataacctgtatactgaatatcacaggaagggtatagacgtcatggcagtggttagatattcttgcacctgctgaagctgagaaaattaaagtaattttttttcctgtggaaagtagaaaatcaagcttttgtatgatttcacacagctttctattctctcttttgttgactctgttaagagtaacatttagtggtggaaactatttcaggatcacacccacaacactagagactgtattaatcacttacacacacataggtatagagtaatcttgaaggggctgtaggccaaagataatgcttttttgaagaattagagactagttaccagcacctggtatttgctgtttcctacagagctgactggacagcctagagtctgctgaggaattcagaggatggccagtagaatgttctttccaccccagaatatttggtagggactcagctgctgtggaatgccaaaaaggctttgagtttgtttcactattcttaagattacacgtaattgtttttttgtaagagattatatatattcaagttgaggatggctttgagttagactttccttaatttggaatcacacagcagatgatacatttatttccatctgataagttacttgatgatgtaaaaagacatttgagttaaagatttttgggaaaaaagctgaatgttgagccatttatgttgtgtactggttccctattcacttggacaattttaagtcttaaaacaatcttaaccatgtgcacaagagatttcacatagtatttggtaattaaattaaggaattctagctcaagtcatgctttttgctgaaatagttgtatatatttagtgcggaaacctgtgttttcaaattaatgtaataaaagtttcaataaaatggaagcctttattaccgtgtttcaaatgctatgctaaaccttttccatttgttattatattaacctcctcatacatagccctactaatttttttactttctattttgaaataattacagatttataggaagttgtgaaaaatagtacagagcccatgttcccttcaccaagtttcacctaatggtagtagctcacataacgataatttaatgtcaagaaccaggaaattgtcatcgttgcaatccataagccttttttagatttcaccagtttcacatgtatttgtgtgtgtgtgtatatatataattgtatgcaattttatcatgtgtagatctggatagccactgtaacagtctatagttctatatacagagctactccatcacctcagggctccctatgctaccactttatagccgcacgcacccttccagcaaccactaatctgtttgcatctctgtaattttgccattttgagaatgttatataaatggaatcatacagaatgtaactttggctttttttcttttaccatacttcctttgagagccatccaaattgctgcatgtatcagtagttcatttctttttactattgagtagtagtccatagtatggctgaaccacacaatttgtttaaccatttacttattgaaggacataccagaagggtggtttccagttttttggctattgcaaataaagctgctataaacattcatgtatataaatatttttatgtgaatataaagttttcattttgggggaataaatgcccaaatgtttggattgtatggtaagtgcatgtttggtttttagagaaactgctgaactatttattttctagaatgactatatcctcttatattcctatcaacaatatatgagatatccagtttctctgcatccttgctagcatttagtgttaccacttttttatttgagcggttctaatatgtgtagtgatagcctgttttgccttatattaatcaataaaaatagcctcatctaatcttaactttttttattttaaaacatcttggcagtattgaactttctcaatgaaaaatctctaaaattgtgacttgaaaggctttaattttccagtttttctttggttttactcttagcagtaacattttaacttttttttgtctttgaagtaattttcagtgtttcctttacatgttgctttttcttagaaactagttactagcatgaagtagatctttagcctcgttttctaaaaacataaaaaagtaaaactgtggggtttatttcaaaattgagagtcctgtcttttcatatgaggatattttatagtctgttggcttggctatattttagggagtaaacctgtggttagtggtttgttgttggtggtggtaaagttttcttacagtatttttatacctgaataatacctttagactctatagaatagatacttgatcttcaaatctatcctagaataaattgttttatctaaacagctttgtgacctgagaattgggacttagtcccttagttttcccttactggccctttgtagtcactgttttgattttgtgaaagtaacttaactcttagcactgtcaggtattgtacattcctgccaaagcaagaataagaatacataggattgtgttttaattctataattaggtgacttttggctaatttccaggaacttggacttaataaagtactagtgataagtttggaaattttagtgtccttgttctttgaagttattcaccctttactttcttgtttgtttggggtgtttatactactgtccctaaatatagctgaaataaaggaagaaaaataacccctgtaatatcactaccaggatataatttcttttttttttttttttttgagatggagtctcgctctgtcgcccaccatctcggctcactgcaagctctgcctcctgggttcacgccattctcctgcctcatcctcccgagtagctgggactacaggcgcccgccaccacacccggcttattttttgtatttttagtagagacggggtttcactgtgttagccaggatggtcttgatctcctgatcttgtgatccacctgcctcggcctcccaaagtactgggattacaggcatgagccaccgcgcccggcctgatataatttctgttaacagtttgatgtaaatattttttgactttttagtgtttttatatatatatatattttatgtttttcttttatcaatacgcactcttactgtgggaataattttaatgtttttaaagagttgggttttatttgtttattttattttatagaaatggggtctcgccgggtgcgatggctcacgcctgtaatcccagtactttgggaggccaaggcaggagcatcacctgaggtcgggagttcgagaccagcctgaccaacatggataaaccgcctctctactaaaaatacaaaattagccgggcgtggtggcacgtgcctgtaattccagctacttgggaggctgaggcaggagaatcacttgaacccggccggtggaggttgcagtgagcaaagattgtgccattgcactccatcctgggcagcaagagtgaaacttcatctcaaaaaaaataaaaaataaaaaagaaagaaagaaagaaatgggatctcaccattttggctggttttgaacttgtggtctcaagcagtcttcctacctcagcatcccaaagtattgggattacaggtgtgagcccatcctgtttgttgttgttcttttgttgttgttgtttttagatgaagtctccctctgtcacccaggctggagtgcagtggcgctatcttggctcactgcaagccccgccacccaagttcaagcaattttctgcctcagcctcccgagtagctgggattacaggcgcccaccaccatacctggctaatttttgtatttttagtggagacgaggtttcaccatattggccaggctagtcttgagctcctgacctcgtgatccacctgcctcggcctcccaaagtgctgggattacaggtgtgagccactgcgcctggcctgttgttgtttaaataaaagaaatttattctcttacagtcgaggccagaacttagaactggttttcaatctaaattttttttcttctttgggagaagggcatcagaatattgtggatatacttttttgacttaaaaaaaaaggttttactgggctgggcatggtggctcacctgggattaactgcctgtaaccttggcactttgggaggctgaggcaggtggatcgcttgagtccaggagttcaagagcagcttgggtgacatggtgaaactccgtctctaccaaaaaaaaaaaattagccaggcatggtgatggcgtgcccttgaagtcccagctacttgggaggcttagctgggaggatcgcttgagaccaagaggcagaggttgcagtgagctaagttcatgccactgcactccagcctgggtgacagagcgagacctcgtctgaaaaatttttttttttttttactaatatgacaaacatcttttcatttcaaatatatttctataccatttttaatatctcattgcctttagaatgaccttgtattcatagtacatatgtatgtgatattccatttatttattttttttcttttgtctttttttggttatattccattgatttaatgtaccttaatttatcttaccaatttcttgttgaccattttgtttccagtcttttgtttttttaccagacatggattaagctgagcctttgccccagacgacattatttcttttttatcagcaaaatatgcgtgtaatgaaattagaattaaaaggcaaaaaaggttatcctttatttttctacttatttttattgagatagtaattcacataccataaatttaacccttttaaagtgtacagttcagtggttttcatatattagaaggttgtacaaccatcgcaactaattccagaacattttcatcaccccagaaagaaactctgaacccattatcactccccactccctcacacaccctaaccctggcagtcacatatagactctctgtctctgtggatttgtttactctggacctttcatataagtggaatcataacagtttgtggccttttgtgcttggcttctcaaacttatctgtttccaaaggttatctgtgtcgtagcatgtgtcagtacttcattcctttttatggctgaatattttattgcatgtatatgccacattttgtttatccattcacctgtagaaggacatttaggttgtttccattttttggctgttatgaatattactgctgtagacgttcatgtacaagtttttatgtgaacgtgttttcatttttcttgggtatatacttaagtgaggaattcctgggtcttaagttaactctctgtttaacattttgaggaactgccaaattattttttaaagtggctgtgacattttatattctaccagcagtgaatgaaatttccaatttctccacatacttgacagcacttttttttttttttttttttgaggtgaagtcttgctttattgcccaggctggagtgcagtagcatgatcttggctcactgcaacctccacctcccaggttcaagcaattcttgtatctctcagcctcccgagtagctgggattacaggcgcatgtcaccatgcctggctaatttttgtattttttatagagacagggttttgccatgttggtcaggctggtcttgaactcctgatttcaagtgatccacctgccttagcctcccagagttctgggattacaggcgtgagccactgcacccagtctgcactttctttattatctgtcttctttattatagccaatctagtgggtatgaagtaagtgtgtcatttgtgattttgattgttagtggtgactaaaaatgttgaatatctttacatgagcttgttggccatgtgcacatctttgttggagaaatatctattcaaatcttttgactattttaaaattgggttatttatctttttattgttgagctataggagttctttattttattttactgagacagggtcttgctctgtcacctaggctggagtgtagtgatgccatcttgactcactgcaacctctgcccccaccccaggctcaagtgatcctcccacctcagtcagcatcccacagctgggaccacaggcgcatgccaccatgcctggctaattttttttttttttttttttttgtattttagtatagacagagtctcaccttattgcccaggctggtctcaaactcctgagctgaagcaatccgcccatctcagcttcccaaagtgctggaattagaggcatgagccactgtgcctggcctattttattttaaagatgaggcctcactttgtcacccaggttggagtgcagtggcgtgatcatagttcactgccattttgccctcctgggctcaaacagtactcacgactcatcttcctgagtagctaggactgcaggcatgtcgctagcatgcccagctaaaacagttctttatattctagatcggggtgtccaatcttttgacttccctgggccacattagaagaagaagaattgtcttgggccacacataaaatacactaacagtaatgacagctgatgagctaaaaaaagaattaccaaaacatctcataatgttttaagaaagtttacaagtttatgttgggccacattcaaagccatcgtgggcctctggccgtgggttggatgagcttgttctaaatgctagacccttatcagatggatggtttgtagatatttatcgcatgctgtgggttttttttttttactttcttttaggtttttttttttcttaaataattaaactgattaaaagctttaatcttttcattttcttgataatgtcttttaaagcacaaagttttgtttcaatgatgtctaatttgtctatttttttttctttggttgcttgtcatacgtaagaaactgttgctaaatccagaatgctgaagatttacttgtgaactttgtttccttctatgagttttatagttttagctcttgtatttaggtctttgatacattttgaggtttttttgttgttgttgagacagtcttgctctgtcgcccaggctggagtgcagtggtgtgatcttggcttactgcaccctctgcctcctcggttcaagcaattctcatgcttcagcacccgagtagctcggattacaggcgtgcaccaccaagcctggctaatttttgtatttttagtaaagagggggcttcaccatgtttgtcaggttggtcttgaactcctgggctcaagcaatcctctcatctcggcctccccaagtgctgggattacaggcatgagccaccacgcccagcctgttttgagttcatttttaaaatatggtgtgaggtagaggtcccatttcattcctttgcctgtgggtatccagttgtcccagaaccatttgttgaaatgactcttgtttcctcattgagcaatgtcgtgagaccctatctccataaaaaataattaaaaaaaaaaaagaatgcagaaggaaacagttttgccaattttgtagtatttactgacaatttgcatatgtctttacattctttagctatttatttttcttttgaattactgcctttgttcatttttcttttggagttgtttgtctttttcttattaatttgtaagagattttgcaaatatatacaatttcttttctcttttttttgagatggagttttgctcttcttgcccaggctggagtgcagtggcatgatcttggcttactgcagcctctgcctcctggtttcaagagattcttctgcctcagcttcctgagtagctgggattacaggtgcccaccaccacacccagctaattttttttttttttttttgtatttttagtagagactcggtttcatcatgttggccagactggtctcaaactcctcacctcagttgatccacccaccttggcctcccaaagtgctgggattacagttgtgagccaccgtgcctggacctcccacattattttgaaacaaattccatatcacataatttcttttttttgagacagagtctcgctctgtcacccaggctggaatgctgtggcgtgacctgtgcttactgtaccttctgcctcctaggttcaagcgattctcctgcctcagtctcctgagtagctgggattacaggcacgcaccaccacacctggctagtttttgtatttttagtagagatggggtttcaacatgttggccaggctggtcttgaactcctggcctcaggtggtccgtccacttcggcctcccaaagtgctgggattacaggcttgagccactgcacccagccaatatcatataatttcatataaatagttctttgtgtatctttagataaggacttaaaagaaggcataatcgtaacaccattattaatacctaaaagaagtgagcaataaataattcatttgccgtatcaaatatccaatgttcatatttcctccattgtcccataataatttttaaaagtttgctcaaatcaaaatccaaacaagattatttcaaagcattgtttgaggtacattttaaatcttaatttatagatttctctgctgtctcttttcccccatatttatttgttgaagaaaccaagcgttgtttcctgtggactttcctactctctggattttgctggttatattcctctggtatcagtttactatgatccctttttcccctgtattttctgtaaatttgtaactagatctagagatttgtttagattttgtgggttttttttttttttttttttttttttgcaaaaatgcatcataaatggtggtgtgtacatctctcagaagacacatatcttaatgtctttttgtggtattagttattaatgattactgcctatatttattaattcattatttggattgtaagtttatgatagtctcttgatgctttttctgttgttagctggaatgcttctaaaaggagaaggctttcctcttcaagctacttggttgtcttgagggtttgcttcttatagggaaagcaggctaagggtgaaaaaggaaatagtttctaactgggtctgttaatgagctgtcaccccaggcaaagagaagcaaggcaggtcacaggaaagtgaagtgggcttgggatgattggtgccccatgcgtgcatgcatgaagggaagttaatcctccctgtagtgaactctactgggcttttggtcagtagccaagactgtcaaggaagacctttgtcagaagccatacctggcctttgcttttagctgttggtagctgaaggaaaccagaacagacctatgacctgtgaacttctgctcagtagacaaagttctctcagcctaaattcagtaagcaggagtaagatgcttgctttcccttgaagtgaaacgtgaattatatgtttcttcaacttgtgctaatattctttttttttttgagatggagtctcacactgtctcccaggctggagtgcagtggtgcaatctccgctcactgcaacctcagcctcccgagtagctgggattacaggcgcctgccaccacgcctggctaattttttgtatttttagcagagatggggtttcactatgttggccaggctggacttgaactcctgacctcacgatctgcctgcctcggtctcccaaagtgctgggattacaggcgtgagccaccacacctgggcaacttgtgctaatattcttaaccttcatgtgaatcattcctgccctcaggctagcataacccatacagccttccttataggaagatttcctactgggagtgaatttgtccagtgattcccccaagatatcccccaatcaaatattttaaaagtcatcatttacatgtaaaaactatgtaacaagcatggtagcagcagcgttaaagaaatggcagtatggcccctgtaagggaaggctccagaagatgagccgcactcagcctctaggtcacagctaccttaggagtttgcagttgttcctggggaagtcagtagacaaagctatctctcaggcctgggcaagatagggattttttttttttctttgagatggagtctcaccctgtcatccaggctggagtgcagcagcatgatctcggttcaccacaacctccacctcctgggttcaagtgattttactgcctcagcctcctgagtagctgggactacaggtgcgggccatcatgcctggctcatttttgtatttttagtagagatggggtttcaccatgttggctaggctagtctcaaactcctgacctcaggtgatccacctgcctcccagagtgctgggattataggcatgagccactgtgcccagtgtttttttttttttaattgtagtgacaggatctcactttgtttcctgggctattcccaaactccaggcctcaagccgtcctcctaccttagcctcccagagtgctggggttacaggtttgacccactgtgcctagtctcagaattcatgtttttaaaagtcactctgtgccaggctcatgcctgtaatcctaatactttgggaggctgaggcaggagggttgcttgagcccaggagtttgagaccagcctggaaaccatagcaaaatcctaactctacaaaaaatacaaaaaatagccaggtgtggtggcatgcacctgtagtcccggttacttgggaggctgaagtgcaaggatcgcttgagcctaggaagttgaggctgcagtgagctgtgatcatgccactgcacaacagcctgggcaacagagtgagaagtaactctggctgtggtggggaaagtggattagtggagaatggaagctgggaaacatggtggttcttgctaagtcagtatcaagggatcacagatgagggggctatttcgtcctaataagggccttggtctcccagatagtcatggatttttctatttagaagctccttctcagtttttcttgcccaaggcatatacggttgatatttgtacaacacaggctggatctgtatgggtccacttatatgtggattttttttcaaccaaacttggattaaaaatatagttgtaggccaggcacagtgacttatgcctgtaagcctagcactttgggagcccaaggcaggcggatcagctgaggtcaggagtttgagaccagcctggccaatgtggtgaaaccatgtgcctactaaaaatacaaaaaatagctgggtgtggtggtgtgcacttgtaatcccagctactcaggaggctgaagccagagaattgcttgaacccgggaggtggaggttgcagtgagctaacgcagcagaggttgcagtgagctaacgcagcagaggttgcagtgagccaacggggtggaggttgcagtgagccaagattgcaccaccacactctagcctgtgtgacagagcaagactctgtctcaaaaataaataaataaaaatacagtgtaggccaggtatagtggctcatgcctataatcccagaactttgagaggccaaggcaggcagatcagttgaagccaggagtttgagaccaacctggctaacatggtagaaccccacctctactaaacagaagtacagaaattaaccaggcataggtggtgcatgcctgtaatcccagctgcttgctaaactgaggcaggagaattgggaggcagaggttgcagtgagctacgattgtgccactggactccagactgggtgacagagcgagactctgtctccaagagaaaaaaaaaaattgtacttacaggacatgaaacccacctgtacggtgtgctgactgggagactggagtatgcatagttcttggtaaacaaggggattcctgaaaccaatcccctgagtatatggagggttgactatatattttaatagaatttattacttttttttttttttttagcagttttaggtttatggaaaaattgagcaggagtacatagtttctctatctccctcacatttccccattactagcatcttgaaatagtgtggtacatttgttacaactgaagagccaaatattgatacattactgttaactaaggtccgtaatttactttagagttcactcttggtgttgcagtttctatgagtgttggcaaatatatcatgacatgtatctagcattatagtatcatattgagtagtttcactgccctaaaaatcccctttgttccaccttttcatccctccatctacctgaacccctgataaccactgatccttttacagtctctatagttttaccttttacagaatgtcatatagttggaatcatacagattggcttctttccatgttccttcctggcttgatagctcttttcttttttttgagatggagtctcgctctcgcccaggctggagtgcagtggcgcaatcttggctcactgcaaactctccgcctcctgggttcaagcaattctcctgtctcagcctcccaagtagcttggactacaggcgcatacctcccctgcctggctaatgtttgtatttttggtaaaggtggggttttaccatattggtcaggctggtctcaaacacctgtcctcaggtgatccacccacctcggcctcccaaagtgctgggattacaggcgtgagccaccctgccctgccagctcttttttttgtactgctgaataatactccattgtataggtgtatgagtttatctattcaccttctgaaggacattttggttgctcctaagttttggcaattatgcatgaagttactataaacatctgtgtgtaggtttttgtgtggtcatgtttttagctcatttggataaataccaaggagcacgattgttggatcgtatggtaagagtatgtttagttttgtaagaaactgccaaactgtctttcagggtgactgtaccattttgcattcccaccagcaatgaatcaagttcctgtcgctccacatcctcgttagcatttggtgttgtcagtgttttggcttttcaccattctaatagatatgtagtgatatcttgtcttactttgcagttctctaatgacgtatgatgttgagcatcttttcatctgcttatttgttgttgttgttgttgtgttgttcattgaaatggaatctcgctctattgcccaggctggagtgcaatggtacaatcttggctcactgcaacctctgcctcctgggttcaagtgattctcctgcctcagctccccaggtagctgggattacaggcgcccgccaccatgcccggctagtttttgtatttttagtagagacaggatttcaccatgttggccaggctggtcttgaactcctgaccttaggtgatctgcccgcctcggcctcccaaagtgctgggattacaggcgtgagccactgcgcctggctttcatctgcttatttgatatgtgtatatgttatttggcaaagtatctgttctgatcttttgcccattttttaatcagattgttcttttattgcttctggggttctttttgtttgctttttttgagacagagtcttgctctgtcgcccagtctggaatgcagtggcatgatctcagctcactgcgacctctgcttcctgggttcaagtgattcttgtgccttagcctcccaaatagctgggattacaagcatgtgccactgcacctggctaatttttgtatttatagtagggacagggttttgccatgttggccaggctggtcttggactcctggtcttcagtgatccacccacctttgcctcccaaagtaatgagattacaggcgtgagccaccatgcccggcttattgttaagttttaagagttctttgtatatgtgtattttttgattcttttaaaattaatacttaataaaataattgtacatatttatgggatgcatgtgatattttgatacatgcatacaatgtggatcaaatcaaggtaattagagtattacctcaaacatttgtcatttctttatgttgggaacatttcaaaatgtctagctattttgaaatatacaataaattattatctataagtcacctcattgtgctgtcaaacattagaacttattctttctacctggctttattttttttaccccttaaccaaccattcttcatcagctccccgtctcccctactctttttttttttttttttttttgatacggagtcgctctgttacccaggctagagtacagtggcacaatctcgactcactgcagcttccgcctcccaggtttaagcaattctctgcctcagcctcccgagtagctgggattacaggcgaatgctaccacacccgactaatttttatatttttagtagagatggggtttcaccatcttagccagactggtcttgaactcttgacctcctgatccacccgcctcagcctcccaaagtgccgggattacaggggtgagccaccatgcctggcccctcttactcttttcttagcctctggtatctatcattctactctctacttctatgagatcaacttttttttagctcccacatatgagtaagaacatgtaatatttctctttctgggtctggcttctttgtatattttggataataagtcttttattagatacgtgttttgcaaatattttttccgagtccgtgacttatcttttcattctcttaaatagtgtcttttgcagagcacacattatacattttagtgcagtccagtttaccaattctttctttgatggattttgcttttggtattgtgtctagaaagtcttcgccaaaccacagtcatctagagttccccttatattatcttacaggagttttatagtttttgttttacatttaggtctgtgatctattttaagttaatttttatgtgaaagatataagatctatgtctggattctctctttttttgagatggagtctcgctttgtcgccaggctgaagtgcagtggcgcgatctcggctcactgcaacctctgactccctggttcaagggattctcctgcctcagcctcccgagtagcacatgacaccacgcccagctaatttttgtattttgagtagagacggggttgcaccatgttggccaggatggtcttgatctcttgacctcgtgatccgcccgcctcagcctcccaaagtgctgagattacaggcatgagccaccacgtccggccagtttttttgttttatttatttatttatttgaaacagggtcttgttctgttgcccaggctgcagtatagtgacaccatcaaggcttcgttgcagccttgacctcctagggtcaagttatcttcttgcttcagcctcctgagtagctgggactacaggtgagcaccactctgaccaggtactttttaaatttattttagagacagggttttaccatgttgcccaggctggtcttgaactcctgggctgaaatgcccctcctaccttggcctcccaaagtgttgggattacagacatgaggcactcagcccagccaatataattcttttttttttttttgagacagtcttactctgttgcccaggctggagtgcagtggcatgatcacagctcactgcaacctctgcctcctggactcaagctgtcctcccacctcagcctcccaagcagctgggattatgggtgcccatgaccacacccagctaagtttttaaatttttttagagattgaatctttctttttctcagcctggtctcaaactcctgagctcatatgatctgcccgcctccgtctcccaaagtgctgcgattacaggcatgaaccactacgcccagcctacaatttatttgtaatccaagttttattgaaaaaaaaatccacatataagtggacatatgtagatccaacctgtgttgttccagtgtcaaccatatataccaataattcttttttttttttttttttttttttttttttgagatggagtcttgctctgtcgcccaggctggagtgcagcggtgcaatctcatctcactgcaacctctgcctcccgggttcaagtaattctcctgcctcagcctcctgagcagctgggactacaggcatgcaccaccacgcccagataatttttgtatttttagtagagatggggtttcaccatattggccaggctggtctcaaactcctgacctcaagtgatccacccgccttggcctcccaaagtgttgggattacaggagtgagccactgtgcctggcctataattctttacgtatattgttagattcagtttgctagtattttatttagcatttgtgtatctgtgttcatgagaggtattgttctgtagttttctttggtttcttttctgtctggtttagggtaatgctggcctcatagaataggttaggaaatatttcctctgcttctgtttctgaaagagaattgaggtaatatctatttttttttttttgagatggaatcttgctctgtcgcctaggctggagtgtagtggcgcaatcttggttcactgcaacctctgcctcccaggttcaagtgattctcctgcctcagtctcctgagtagctagaattacaggcatgcaccaccatgcctggctaatttttgtatttttagtagagatggggtttcactatgtgggccaggctggtcttgaacttctgatctcaggtgatccacctgtcttgtcctcccaatgtgctgggattacaggcgtgagtcactgtgcctggcccgagataatatctaatttaacagtttggtagaattcaccagtgaacccatctgggcctggtgccttttgctttagaaggttattgattattgattcaatttccttaatagataaaggtgcattgagattgtcttttcttcttgggtaagttttaatacattgtgtctttcaagaaattgttccatttcatctaggttatcaaatttgtgggattagagtccttcataatatttctttgttttgcttttggtgtccataggttcagaagtgatggccctttttcattttttctattagtaatttgtgtctttgcccttttttttctttgttaatctggctagaagcttatcaattttgttgatcttttcaaagaaccagtttttggtttcactgatttttctctattaattttgttttcaatttaattgatttctgctctaattggttttcttctgctcactttggatttaattttttttagtttttctagaaaactaagtttttaagtgaaaactgagattattgatttttagatcttttttctaatgtttacagttaacactgtacaatttcctgtaagcactgctttctctatatcttacaaattttgatgtcatattttcattttcatttagttagaaatatctcttgagacttctttgacccatctgttatttagaagtgtattgtttaatctccaagtatgtattttgggatttttctggctatctttctgctgttgatttctagtttaattacatgtggtctgagagcataccttgtatgctttctattcttttcaatttgttaaggtgctctttgtggctcaaggtggtctactttttttttttttttttttttaaagaaaagctggccaggtgcagtggcttatgcctgtactccagcactttgggaggcgtaagtgggaggatcacttgaggtcaggagtttgagaccagcctgggcaacatatagagacttcacttgcacaacaaatttttaaaatattagttgggtatggtggcatatacctgtatatggctgaagtgggaggattgcttgagccctggaggttgaggctacatgagccatgatcgcaccactgtactccagcctgggcaacagagtgaaattttgttctctcttgaaaagaaaaaaaaagttgatgacataaagttcattcatcttttttgtatgtgacttcaaaataactactgatggttaaaaaaaaaatcagaatgatgcaacccaagtgtccatcaatggatgaatagataatatgtggtgtatgaatacaatgggctactattattcagcctttaaaaataagaaaatgctgacactgctgtaacatggatgaacgttcagatcattatgctaaatgagaaaagccagacacaaaaggacaaatattgcatgattgcacttatatgaggtatctggaatataagagtcatagaaacagtaattcagtaattagaataatgcttgccagggcctgtggggaggagggaatgaggaattcatgtttaatgggtacagagtttcatttggaaaagattaaaaagttatggaggtggatggtggtgaggattgcacaacagtgtgaatgtacttaataccactgaactgtacacctaaaaatgattaaaatggtacattttatgttacatatgttttacaacaatttttacagatggaaaaaaattataaaaaacatcaggatggtgttgacagtgaaaaggttaaagagttactttaaaaatttactttattccagccgggtgcggtggctcacacctgtaatcccagcactttgggagaccgaggcgggtggatcacctgaggtcaggagtttgagaccagcctgaccaacatggagaaaccccgtctctactaaaaatacaaaattagccaggtgtggtggcgcatgcctgtaatcccagctacttgggaggctgaggcaggagaatcgcttgaacccgggtggtggaggttgcagtgagccgagatcttgccattgcactccagcctgggcaacaaagcgcaactccgtctcaaaagaaaattttttttttttactttatcccaaatgtttatatttactttggggcttatgtgaccagtttaattttcatttgtaattgacttgatagaacacactaatgttcagttaagatttcttatggtgtggtgaggagtaggatttttatgtaaataagcccaaaattgtatatatgaggttaatctgatatttgcagaagatattcatgcattactgtaaggaccactctgcttattcatttgaccgatttgttacaacatggttagaaatcatcaaggtgtttgagatcaaaggatcttcagaggtgatttactccaatccttttttaaaaaattaataacttgagcctcagagaagttaagtgacattaccaagttctgctgttagtatagtgactttatctttacctgaatccagggtttcttagccctagtctgtaatgtgtcctagtgtgcctctagaatctggtcctgtcagcccaagtctgttaaatcaaataaaaccagggcttggtgcttcaccttgtcttctgccataccgttgggtttcctgtggaccatgcagataatgatgatgggctcagtgggcttgatagtgataactcctaaagcagctccttctaagtgcggttctcaatctgaggaagttaaaaaaaaaattagtgactagaacccacttctcaggtactctgataaaatacatttgtaggggagtgatagttttcactttctttttttctttttcttttttttttgagatggcgtctcgctcttttgcccaggctggagtgcaatggtgcgatctcagcttactgcaacctctgcctcccagtttcaagtgattcttctgccttagcttcctgagtagctgagattacaggtgtgtgccaccatgcctggctaatttttgtatttttattagagacgggatttcaccatggttggtcaggttggtctcgaactcctaaccttgtgatctgcctgcctcagcctcccagagtgctgggattacaggcatgagccactgtgcccagccttcacatattttttgaaataataggccagttgcggtggttcatgcctgtaatctcacactttgggaggccgaggtgggcagatcacttgaggccaggagttcaaggccaccctggccaacatggcgaaaccctgtttctactaaaaatacaaaaaaattagccgggtatgatggcatgtgcctgtggtcccagctactctggaagttgaggcatgagaatcgctttaacttaggaggtggaggttgcagtgaggcaagatcgtgcccctgcactccagcctgggtgacagagcgagactccatctcaaaaaaaaaaaaaaaaatcgtatgcagtaaaggttgaaaactgctgcccaaaggcgctattaaactataggttcccaaacctggccaattgtcaaaatccctcaagaaggggcagtggggtctaagggaccccattgctgtagaggagattagtagtccaagagtgagatgaactgttggaagtcctcaaacttccaaactattaaaatagaatagttttgcttccttaaaatagaatagttttcttcctcactgatttttctgtattgattagaaccataacaagtgaattaaacaactacaaaatagttatgtgggccacagacattattgtaatgaagtgaagtttggctcaggccttgtaacacaattgctttttggattaaaagtaaaaatattaaattgtgaaattatgtgtaagttttaaaaaattggtcttgtacaaaagtgttgggtttttctttgtttttaactggatttgtttttaagcaagacagaatatttatattgttggagagtcacaaaggaggtgtgtttgtggatttaaatgtggagacagtgtgccttgaaatgccctttatcagtctgattcaagccacctgcaatcatggatttgaacttttttttttttttcccctgagatagggttgcctaggctggggtgcagtggtgtgatcttggctcactgcaacctctggctcagcctcttggagtacctgggactataggcacacaacatcatatccagctattgtatttttttttttttttttttttatagagacagagtttcaccatgttgtccaggctggtcttgaactcctgagctcaagcaactcacctgcctcggccttccagagtgctgagattataggtgtgagccaccatgcccagccttcactttatttaaaaaccatagtttttaaaagccacattcctactgatgaacacagaggttgtttccagtgttttcatttgcagggctgtagtgattgtttttgcacatgcctctttatgtacatgtgctgtgtcttctgggacagagagtggacattttaagtgttttattggtcctgctaaattgtctttcagccaaactgctgcagaggtgatggagatgaggtgggtactcaggagaattatgctcagtgcttgtgtgctagtcactgacctggaaacattttattaaaaatgctagattaggttagtgatgtaaataccaggtgatagtaaccagaataattgtgtcaaaacatcaagaatcatcaagagatgccaggcatggtggctcatgcctgtaatctcagccctttagtaggaggccgaggtgggcagattgcttgagctcaggagttcaagaccagcctaggcaacatggtgaaaccctgtctctaccaaaaatacaaaaatttgctggatgcagtggtacgtgcatgtggtcccagctactcagtaggctgaggcgggaggatcgcttgagcctgggaggcagaggttgcagtgagccaagactacactacagcccgggcaacagagtgaaaccctgccataaaaaaaggagaaaaagaatcatcaagagaaaacttaatttgatgtgctctgcgttttctttggtgcttcatgtcagtgttagaaactataggttgtatatttattaatttttctcctacatttgttcaacttgactaaaatattaactccaaatgcctagaatttcaaaatacctcttcgttataaagtatcaactatttcttagtccccttaagctgatagtattgtgtcattgtaaaagatcccttgtgaaaaataatttttgtcaacatgaaaggtcttaatgtgtctcctagtttacattttacatggtcttttccatgtatttatatagttgacatatatagctttttttgtaaatacactttcctatgtgaacatgccaaggtttacttaagcattctcttattcttggacatctaaattgcttcttattttctattgtaaataaagtgctagaagcttctttcctgaaaagttatttacttttcacctagtatttccatctgtgttcctcaaaataagattgctgagattcatgtatgttgccagaaagattgtgaggtttaacagtgaatgaggaaaacttttaacactcaaggtctaccaagtacagcaagttttattttacctttttttttttttttttttttttttttttttgagttaggccaagttgcctaggctgggctcaagcaatccttctgcctcagcctccggagtagctgggattataggtgtgcagcaccacacccggctttcttgtattttttttttatttttatttatttatttatttttgagatggagtttcactcttgtcacccaggctggagtgcaatggcccaatctcgactcaccgcaacctctgcctcccaggttcaagcgattcttctgcctcagcctcccaagtagctgggattacaggcatgtgccaccacacccggctaatttttctatttttagtagagacggggttcctccatgttggtcaggctggtctcaaactcccgacctcaggtgatccacccacctgagcctcccaaagtgctgggattacaggtgtgagccaccgtgcctggctgctttcttgtattttatcttgctgtagcggttgtgggatttttgcctggtgtgttcattggagtgtgtgtgtgtgttttgagatgtatgactgtgttggttgtgttgtcatactttgaaggttcattaatgtgctggtctttctatttttcttgttatacttagtcacttaaaaacccttttttttttttttttggaattcttatttttacagtactttgaattcctgttttattaatcaccttcttatctgaaagtgaagtaatagtgtacttggcaccattgaattagaaaattgtgtgtccttggccagaagatcacatacacaggaactcgataagttgagagatttagccgtttcagaaatgggcatttgtgtcttccagtggagaagcatctgcaaaagaattgggcagatttggccaggcgcggtagctcatgcctgtaatcccagcactttgggaggccgaggtgggcagatcacctgaggtcaggagtttgagaccagcctggccaacatggtgaaaccccgtctctactaaaaatacaaaattagccaggcgtggtggctaacccatttaaagtgggtgaattatatcttttatgttttaatgtattcctagagttgtgcaaccatcaccagaataagttgtagaacattttcatcaacccaaaaagaaactttgtattcattagtcgttcctcctcatttctcccctaacctcccagcactaggcaaccatcagaatactttttgtctccacggatttgactgttttggacatttcatattaatggaatgcacaatacagtagtataatacattaattttggaaggccaaaattaatggcttttgatgtctgccttttttcacttagaataatgtcttccaggtccatctgtgttatagcatgtatcattactttatcctttatgtggctgggtaatattccattgtatggttataccatgttttgtttatctattcatcagttgatggacatttaggttgttttccattgactattaagagtaatgctgccggccgggcgcggtggctcacgcctgtaatcccagcactttgggaggctgaggcgggtggatacgagggtcaggagatcgagaccatcctggctaacacggtgaaaccccgtctctactaaaaaacaaaaaattagccgggcgtggtggcgggcgcctatagtcccagctacgcaggaggctgaggcaggagaatggcgtaaacccgggaggcggagctggcagtgagccgagatggcgccactgcactccagcctgggcgacagagcgagactccatctcaaaaagaaaaaaaaaagagaataatgctgccatcatgcttgagggtttttttttattttgttttgttttgtttgtggggggagcggagggcacagtctcactctgttacgcaggctggagtgtagtaggcttactgcagcctcctccgccccctggttctagtgattcttgtgcctcaggctcctgagtatcatgctgcaatttttgtgtgagcatacatttttcatttctcttgggtcaatatctaggagtatccccacttttggttctgagtgttaattgagcctgttgtgttctcacattccctgtacttagatggaaatagtgctttgcctaaaaagaaaataaaagacctgattgtgcaggcgagtgagaaagagagagaggcgctagggttatttccaccctgatactctttgggtagtcttggtatgactagcaaagaagcaagctccaagttgtagtttgcttccaagtttctggcttctgtgggaatttctgcatctaagtaatgacaattttcagttacttgcagtaagtaaattatcaaccaatcttactgtgtattactagcctagtaggaatttacttgtatatcgagaggaatgctgcagctttcaccttacttctaatggggattaatgcttacttaacttgcagttttggaggcaagtacaaagtacaggaccaattatggtcatgaagtgagagagaagtctggctagtgaatggtgattggcaactccagttgactgttcatggcatcttagatctgtgaggagggaggagggaaggaaagttcaagctggtctttatggtaagttctggaacatttccctgtgtcaatgggtcatctgttcattcactgtgtaaaatggttgagggaagttttaatttacatgcttccttattgtgtaaacctttgatttttagtgatttcagagtttgtttttataattacttaacacgtgaagaggatgcagagtaacgtatcgaagctctggttaccttccactgggatttgacacatttgatttcctttattccctccttccttcccttcctccctctctcttttcttaaggaatgcaactactcagattcacctgcacacccttggcatacctctcactcccccttacccccacttcctcagaggtgacctgtcctcagaggcaaatgtgtgccctttccatccgtacttttatgctctcatctatgtttacatctattagtacactattgtctgtatttttaaacattacataaatggtgtaatttttacttttaattctgtagtggtgtttctcaaattaagttctgcacaaaacatttttatagatatccagtgttagcttaacgtttttcttattgtggtaaaatatacataagataaaatttaccattttagccatttttaagtttacaagtcagtggcattaagtacattcacagtgttgtataaccatcaccattgtccattgccagaacttttcatcatcctaaacagaaactctgtactcattaaacaatagtttatcactccttccctgcaactagatgctggcaatcaccattctactttctatctctatcaatttgcctcttccatctaagtggaatcctacatatttgtcgctttgtttctggcttttttttcttcttgtgatgctttgttttaattatgttcctggctttcatcatctagcaggctgattccaaggtttgtccatgtggtagcttgtatcactttaatgtttttagagatagttaatattacattgtttatatataccacattttgttttttcattcaaccttgatggacatttgaattgtttccccctttacctgttgtgaataatgctgccgtgaacattgataaccaaatatttgtttgaatctctgatttcagttcttttggttccatacctaggagtggaattgctggatcatatgataattctatgtttaactctttgagggatggccagacttttccaccatagctaaatcattttaccttcccacaagcaaagttcaagggctccagtctctccccataaggtcctttgcacttttttttttgagacggaatctcactctgttgcccaggctggagaacagtggcaccatcttgcctgacctcaagtggcacctgccttggcctcccaaagtgctaggattgtaggcgtgggccactgcactctgccaattttttaatttttattttcatttatttttctttttttaatttttaatttttttattttttgaagggataaggtctcactttgttgcccaggctggtcttgaactcctggcttcaagcaatcctcctacctcggcttctcagagtgctgagattataggtgtaagcccctgcacatggcctttactctcttgatagtgtcctttgatgcacaaaagctttcaattttgatgaagtttattttttatcttgttacctgtacatttggtgtcatatatctaagagaccattgccaaatgcagtgtcatgaagctttccctcagtgttttctttctgcagttttatgattttagctcctaagtttaggtctttgatccattttgagttaatttttgtatacagtttgagagtcacacttgaggctctgggcgcagtggctcacgcctgtaatcccactacttggggaggccgaggcggatggatcacctgaggtcaggagtttgagaccagcctggccaacatgccgaaaccctgtctctactaaaaatacaaaaattagccaggcatggtgatgcatacctgtggtcccagctacttgggaggctgaggcaggagaattgcttgaacacaggaggcggaggttgcagtgagccgagattgtgccactgcactccagcctgggcgacagagcgagactccatcgtgcggggtggggggtaaaagtcaaacttgagtcttttgcctgtggatatccagttttcccatcactatttgttgaaaagactatcctttctcaactgtgaatggtcttggtaccctagctgaagttatttttattattatgttacttaggaatgcacataaggcctggcacggtggctcatgcttgtaattgcagcactttgagaggtcaaggtgggaggattccttgagccgaggagtttgagaccagcctgggcaatatagcagcaagaccccatctctatattttaaaaaaagaagaaaaaaaaacctctgatgcataaaatatttaaacttgtatgcattcttttctttctttattttttaaaaattgagacagcagcttactctgttgtccaggctggtcttgaactcctgggctcaagcagttcctctcaccttggcctgcagtgctgagatgacaggtgtgaaccactacacctggcctgcttacagattataaaaagaaaataagtttacaagttaaagacagataaaatgacaaaatcagtaaaattaaaattacttttatggagccgatgatgtttattccagttgctcctctcattgtgaatatggtattgttgctgtggcagatttggaggccgtggcagatttggaggctttggcaatggcttctgttaccttgccatgaggtaactcagttccctcatcacttttctctgagaactataaaaccttggaggggtgccttctgcccttcgcttggcatgtatattatgcagggatcaggtcttactccgttcttgattgttagtacaaattagttaaaattgtattgtttggccttagcctgatggtaaacacaacagcacacgtgggctgtgaaatctctgggcagctctgtgtttctagggaagcatctcgatgatccagaacaggcttatactaatgttttagtgtaattttgaaatgaaaacacagcatttaaaaattcttatagagaatgtatagaccttgagaagtgttagcagacccagtttacgacatgtctcaatattatgaaacattgctttattccctatcctgcttgtacatttaattttttcatccagttttaaacaacttgggtactgtggcctgtgcctgtattcccagctactagggaggctgaggcaggaggattgcttgagcacaggactttgagggctgtagtgagctgtgattgtgcctgtgaatagccattgtgctccagcctgggcaacatagcaagaccctgataccttgggtttttaaaaaacaaaacaagatacatgctgacatttctggtttggcaggcagagcttgttctgctccccaccctcccttttcccatagtaaccatttataggacatctcactgttgtctactctgtgttgcctctgcttccctgcgtggtagatctaggaatcttaggatttcttagttttagctggtgatccgtatctttttcttaattccattgtaacttcagcttttcttattgcttgtaggaaggctgtttccattgaatacaaacaaaataaaagcttttattcttaatcttagagataggatgtttgtatttaaaaataattgtgctgtcaaaattctgtcaagttggcttttaccacattagttttttttaatgtggtttatatgaccctggagtaccttgtcttctcactgttaaattctcaactgagttgtccctatttaaagtgtgagactgtgccagtttgattttaaaatattgcaagtgcgttatggcaagataaaactgcaaagaaagaaccttcatgtccctttgattataaatgcttttggcacttgtttctactttttcctaatgttttttgaggaaagaacctccaactctccagacaggtctgggggcaaatgactaaaacatgaactgaggccctgggctgtctctgtgaggatatcccctctattctctctgaaatgtcccagcatgtggtgcatttcttgttagtgtggactcctctgtatataacacatcttatttatcttctgtgcataacatgaagtagtgccctaatgcaattccaggatgtaattcagcatttctataaaaatacagtgtttttctacatttgcatcaaaaaataaccagataattatatttattaagaaaatagcatttttggctgggtgtggtagctcacgtaatcccagcactttgggaggccgaggcaggcagatcacttgaggtcaggagtgaggcaggcagatcacttgagatcaggagttcgagaccagcctggccaacatggtgaaaccccatctctactaaaaatgcaaaattagcctggcgtagtggtgcatgcctgtaatcctagctactcaggagactgaggcaggagaatcacttgaacttgggaaggggagattgcagtgagctgagattgtgccactgcactccagcctaggcaacagagtgagactctgtttcaaaaaaaaaaaaaaaaaacaaagaaaagaaaagaaagaaagaaatgtatttttggtatttgttttcacaaactagagcatttatgtgaaataacattgctagtattgatattataccatagtataatacttagttcttcagcgatgtatctctgctgatcagctacatgatatctacttgagctgttggattttttttaagaacagtgcatttttgaatgcttttgaaaaattgtagtaaaatacataaaacaacatttaccttgtaagcattttaattggtacaattcagtgacattaagtacagtcccagtttagtgcaaccactgttactgtctagtttcagaacgtttttgccccagatggatactctgtacctgttgaacattcagtcctcatggcccaataatctttatgtctgtatagatttgcctattctgcatattttatataaatggaatcatgtcttttgtgtctggcttcttttacctagcatagtattttcaaggttcatccatgttgcagcatgtttcaatactttgttcctttttatgtccattgtatggatatgccacatttcgttaatgacaattcttttgggtagctacattttaaaacattatagtagaatacatatagcatacaatttactatcttaaccatttaagcctgcagttcagtggcattaaatacattcacgttactgtgcaattatcaccaccatctgtttgcagaaacttttcatctcctccatttgaaactctgtatacattaaacatgaactctcccttctccccttcctccagccctggcagccaccgttctacatttttatctttctgacagagattttactactctaggtacctcacataagtgaaatcaaccagtatttatccttttgtgaccagcttatttcattagcttaatgtcctcaaggttcatccatgttgtagcatatgtcaacatgactttccttttaaggttgaataatattccattgtatgtatatgtcacaatttgtttctccatttatccatcactggacatttgggttgcttttacctatcggctgtcttgaatcatgttgctatagctgtacaagtatctatttgagtttctgctatcaattctttaagtatatgcccagaagtggaattgctggatcatatggtaattccgtgtctggttttttttttgaggaagtgccatgctgttttccacacagctgtaccattgtacattccccccagcaatgtacgagggctctgatttcttcacatccttgctaacacttagtattttttttgatagaatagccatcctaatggctacttttaaagtatgtttaacattatttatttatttttaattttttttgtagagatggcatcttactgtgttgcccaggctggtcatgaactcctgggctcaagcagtcctcctgcttcagcctcccaaagtgttcggattataggcgtgagccaccatgcccagcccaaatttaaatatataactaaacacatagcagctaacaccaagcctttaaaaatatcattaataggccaggcgcagtggctcatgcctgtaatcccagcactttgggaggccgaggcgggcagatcacctgaggtcgcgagttcgagaccagcctgaccaacatggagaaaccctgtctctactaaaaatacaaaattagccgggcatggtggcacatgcctgtaatcccagctactggggaggctgaggcaggagaatcacttgaacctgggaggcggaggttgcggtgagtcgagatcgcaccattgccctccagcctgggcaacaagagcaaaactccatctcaaaaaaacaaacaaacaaacaaaatatatatatcattaataggccgggcatggtgtctcacgcttgtaatcccagtgctttgagaggccgaggtgggcagatcactggggtcaggagttcgataccagcctgggcaacatggtgaaaccctgtctctgctaaaaatacaaaaattagccacgcatggtggtaagcacctgtaatcccagctactcaggaggctgaggctggagaatgcttggacctgggaggtggaggctacagtgagctgagatcacactccagcctgggtgacagagcaagactctgtctcaaaaaaaaaaaaaaaaatcattaataaatgtgatcttttttcttcctatacaacaagttgtcaagcaagtatgaccttcttaattgaccctttgacatgaactgggatgagatcgtggaggatgttgaggagacagttgttaccatagtgcacttctaaaaactttaattctatagatttctttaaaattttttttaaaattattatgagtacacaataggtgcatctatagatttcattaccctcaaataaatgtacaaggcaatgcagagaaatgcacagtgtaacttggtagacttgacctatcaagttactgttgaatatattatggagcctgtgtattaccaggggcagcagacttttcctgtaaagatagttgttttcagctttgttgaatctgtggtctctgtcttaactacaactgagaaagccattgacaatatatagatgaatgtacatgactattctaataaaactgtgtacactgtaatttgaattgcacataattttcatgtgtctcctgtataattcttcttttgacttcttttcaaccattaaaaaatgtaaaaacaggccaggcgtggtggctcatgcttgtaatcccagcactttgggaggctgggtggattgctggagcccaggagcttgagatcagcctgagcaatgtgatgaaaccctgtctctacaaaaaattagctgggcatggtgttatgtgcctgtggtcccagctacttgggaggctgaggtaggaggattgcctgaacccgaggaagtcaaggctacagtggtttgtgccactgcactctagcctaggtgacagagtgagaccctgtctcagtgaatgaatgaatacattattagcttgtggactatacaaaaatcagaggctggaggtgagctgggtatggccattgggtgtggtttgctgacttctggtagagaggaattaggagatgttaaaggtggtggaactgtcagatacttgcattcttttagaaatactttggagttagctttttggttcaggcaaaggaccaaagggttaggagagtcaggccggtaaagaggagtggtgggcccatagcagcagttcctggagttttttttttttttttttttttgtgagacggagttttgctctgtcgcccaggctggagtgcagtggcacgatctcggctcactgcaacctctgcctcctgggctcaagcaattctcctgcctcagcctcccgagtagctgggactacaggtgcccaccgccacgcccggccaatttttttctatttttagtagagatgggatttcaccgtgtttgctaggatggtctcgatctcctgacctcgtgatccacctgcctcggcctcccaaagtgttgggattacaggtgtgagtcaccgcgcctggccaggtcctggagtctttaagaggaggtttgtctgatggttggttggacaaaagcctgggcatgttgtcaccttccataagtgtttgtgggaatgtaggtaatgaggaggagtaaaggattcctgaaggatgaggaggagggctggtggctgccataggaagtgatcactgttttggcagacctgtcttagagtaatgaccgtcatactctctcattgcccttgtgaactcatgaaatcccatggctgctaaagctgaaggtcaagtggggacttcccggccactgggcttagcaccccacagagctgtggagtgggcattaatgtcccttttttatagatgcggagactgagaatgaggactgttggtaacttttgaaagggcactcagctagaaaagtctgagccaggatttcaagtcccatggctttacctctgtggtcctaatatttggtgtgttcaagtgagatctgtttttttcctatttcattttgattattgattttcataaatttttttctcttttgagatagtatcttctctctcttttcttttttctgttctttctttctcttttctttctttttttttttttctctgagacggagtcttgctctgtcgcccaggctggagtgcaatggtgcaatctcagttcactgcaacctctgcctctcgggttcaagcgattctcccatctcagcctcctgagttgctgagattacaggcacctgccatcttgcctggctagtttttgtatttttgtagagacggggtttcatcacgttggccaggctggtcttgaacttctgacctcaggcgatccacccgcctctgcctcccaaagcgctgtgattatatgcatgagccaccatgcctggccattatttctttctttctttctttttctttttttcaggttcattgaatttgctttgagacagggtcttgctctgttgcccaggctgaagcgcagtggtgcagtcatggctcactggagcatcaatttcctgggctcaagcgatacttgcacttcagcacccctccacccccacccgctcctttcccccacagtagctggaactacaggcgctagccaccgtgcttggctaattttttttttttttttttttttgagacggagtctcgctctgtcacccaggctggagtgcagtggcgcgatctcggctcactgcaagctctgcctcccaggttcacatcattctcctgcctcagcctcccaagtagctgggactacaggcgcccgccaccatgcccggctaattttttgtattttttagtagagacggggtttcaccgtgttagccaggatggtctcgatctcctgaccttgtgatccgcctgcctcggcctcccaaagtgctgggattacaggcatgagccactgcacccggcctggctaatttttaaatttttttttgtagactgcccaggcttgtttaattgattttctatgtgatcttagggaaatcgattatttcccataaacatttttttaattagaagttaaattctgcctagtttgattcacaggattattgtggatgactgaaacagagaataggtaagagcttctttgaaaaatatgaggtaccatacagaagttagatgctttgtcctggtgataccccctccaaagcacagctaaggaaatgtggaaggcactcttatctcatcatatagctttgaaagcctagcattgaaagtacgaacttgattcttttggagaaatcctttggctctcagtgagtttactttctattaatgactgtgttaagcggaatgaaaactgaaagaggaaaggggaggaagtcagaattaagcaggaagagtgagcccatagcagagtccagatttagaccccaagctacttggaatgatactggacaattatgggtgtgtttaatgatggtcctgagtcatgaaaacaaaaggaggctttaaattatgtctggcttagtgtacagcatatttttgtcattattcaagttttagcatgtaaagaggaaagtgtgcagtacttatgcatatcattttcattaatgaaactaaatgaggcctctttaaaattatcagtgttcacagtatcttccaaaagacatgtaaatgtataaaggtataaaaaatatacatataaattttacaattttgtgagctatatagtagatctcttattttgtccataggtcttaaagatcttatactgtattcaggaataaagataacttcagtgggaggcctttacagggctaatgagtaagcattattttgataaagttctgtgttgtctacaatagatatagtagaaatactcttggaatggtaatcatcccaggccctgctttggagcggaagaaatagtcaatgtagaactttacagtatattgtacacagatgtgcctgctaataacttctgtagacagcaaagtttaagagaaattaggtggtaaatgcaacatatgtatctaaataaatttggtctgagggatttgataagatgaaacagtacatagtccagaaaatttttatactcaaagaattatagaaaatatctgaaatgttttcagttttgtgcatatccagaaaatgtcatcctgtgatctgctggttggcagcccaatggcagtattagatgtattgtttttattttgttttgtttgctatttatttggttaagagagttacctaattaggagtgtgaaaaaaaagatttattatagtagtgggcttttgtttgacttaaaacatttttgttgttaccacagtatgagtgccttgtttgtgaaatttgtttaccgggaagccatatacttagagtagcttttagtttatcattatcatcatcatcatcatcatcatcatcatcatcatcatctccttcatcatgaaaggaagaagctaccaatgttgctttattctgcaaaaaatacaatagatgcttgttgaaagtatggagtgaaatcttaaatatgtctgttaaaaagagtacaactggccaggggtagtgcctcatgcctgtaatcccagcactttgggaggccaaggcgggcagatcgcttgagccaggagtttgagaccagcctgggcaacatggtgaaatcctgtctctacaaaaaaaaaaaaaatagacaaaaattagctgggtgtgatggcatgcagctgtagtcccagctacagtggggctgaggcagggggattgcttgagcccaggaagtaaaggctgcagtgagctatggttgtgccactgcattccagtgtgggtaacagaacgaaaccctgtctcaaaaaaaaaaaaatagtacaactttaagcaggatgtgggcacatgcctgtagtctcagctacttgggaggctaagtcaggagagtcacttgagcccaggagcttgatgctgcagtgagatgtgattgtgccactgccttccagcctggggatgatagcaagaccccatctctaaaaaaaaaacaaaaaacaaaaaaaaaacagagtacaacaacctttggtaaacttggaatataaaggtgtttccttaacctgttaaagagctgataaagagtggtactttcaaaccagtacacattatgtgaaacactagagacacttcccatttgttaaaagaaaaaccttagccaaattaaatttaagttttttttggagacagagtcttgctttgtcacccaggctggagtgcagtggtgcaagcacagctcactgcaacctccgccttctgggttcaagtgattttcctgcctcagcctcctgcgtagctgggactacaggtgtgcaccaccacgcctgggtgatttttgtattttttgtagacatggggttttgccatgttgcccagtctggtcttgaactcctgggctcaagcaatctgcctgcctgggcctctgaaagtgctgggattacaggcgtgagccaccatgccatttaatggtttaattgagcaaagaatgatttgcaaattgggcagcctcccgagccagagtaggttcagagactccagcacagccatgtcgtggaaaaagatttatgaatggaaagaggaaagtgatgtaccgaaaacggaagtgaggtacagaaacagccggattggttacagctctgaatttgccttatttgaacacaagttgaggtttgtacagttggccacctttgattggccaaaactcggtgattggcacaagagcaggttatagtctgtttacatctccattttggttatagttcattatggacagaaaaacctgtaggtcaaacttaaaatatgtaaggagacagttttaggctaaacttgatttaacacattaaatccgtaacaagacaggatgcctgccctcaccatgttatttgatcttattttagtaattctagccaatgtagtagggcaagaaaactgcctgcttggctacaaaataaacacacaaaagtcagtatctgtaatatgtgacagaaaatataattaaaaaaaaaaaaaaaagccgggcacagtggcccatgactgtaatcctagcactttgggaggccaacgtgggtggatctcttgagctcaggagttcaagaccatcctaggcaacatggcaaaaccccgtctctacaaaaaatacaaaaaaattagccacgtgtggtggtgagctcctatagtcccagctactttggaggctgaggttggaggatcatttgaacctgagaagcacaggttatagtgagctgagatcacgccactgcactccagcctgggtgacaaagtgagactctgtctaaaaaaaaaaaaaaaagaagaaaaggaaaggaaagtaaaagaaaaaataatttcacttactagagcatcaaatccctaagataatttagagcaaagccagaaaagtgaagaaaatataaaaatctttacagtgggttgcattttaaaaaaaaacttgaagatatcagatcaactcactaatgtattaattaatataatttaaattaaaatcccactgatttttttgtggggagggtgggagagtcatttgttaaaatgattctaaagagcatctggaagaataagcaggcaagaatagccaagaacattttgaaaactaaagatgagtttggaagacgattggttttgtactgtcaactacttatagattttacatgaattttaaagggtaatctgagccctcgaatagacagaaatagccatagatctgaaagaacacttaagacctgatccagctatccatgagagtatatataatcaaagtctttgtgtgtgaatcaggagtctttcaggtgcaaggtaaataaactcataattgcttacgcaaaggtggtatttgctttagtgactccagagaaagctcaagtgcctatctctcccctgactttgattctttttggggttggctccattctctcctgttgctaatggcttcctttgtgcagccagaggaaaggaggtgtggttttttgatacttccaggcttctatttttatagcttgagatcaaagagggaagtgaccttccttagagtcagtgtgtaaagtcctaaggaagataccacgtggggtgctggggccatgtgcccatccctggcccatgaccatggggatgctacactaactgggggccacgcatggctgttcctgccctgaactgccagctggctttgcagtgcagcctcaccagaatcacatggaatagtagggatatgaattgtttcccaaagaaagtgtgtggggtggtagaattactagtgggggagtaaggggacaggccattgggcatactggagcagcatttactcagtcattgagaaaaggatggaacattcaataaagggtgctggacacatttgtgctctaaaaattttgtgtttcacctattaatttatccctccccttagcccctggcaaacactgatctgtttactgtctccatagttttgcctttcccagaatgtcacacccttggaatcatacagcatgtaaccttttcagattggcttcttttacgtagtaatatgcatttaggattccttcatgccttttcctggattgatagctcatttctttttagtcctgaataatattccattctatggatataccacaattgatccattcacctactgaaggtcattttgattgcttccaagttttgataatttaaaaaattttttaagacagggtgtcattgtgttttccatactggtctcctgaacacctgggctgatgtgaacccctctcctcagcctcctgggtaactgggattacagctatacaccactgtgcccagtgtgacaattatgaataaggctgctgtaaacttctgtgtaggtttttttgtgtgtggacattggttttcagttcattatggtaaataccaaggagtgcagttgctggattgtatggtaaaagtatgtttagtttgctaaggaactgccagctgggtgtggtggctcatgcctgtaatcctagcataatgggaggctgagacaggaggatcccttgaagccaggagttcgagactagcctgggcaacatagtgagacctcatctctacaaaaaatttaaaaattagctgggcgtggtcttatgtgcctatagtcctaactgcttgggagactgaggtgggaggatcacttgagcccaggagctggaggtggcagtaaactgtgatcataccactgcactgctgcctgggtgacaaagcaagaccctgacttaaaaaaaaaaagagaaaagaaaaaaagatgagtcagagggtaaggaagcaaaaataagtaaataaataaatagaagagaaaagaaaaaagaaaaaactgtctttcaaagtggctgcgccattttgcattcctaccagcaatgaatgagagtctgttgttgcacatcctcaccagcatttggtgttgtcagtgttctggattttgaatattctattaggtatataatgctgtctcacttgttttaatcaatgatatatgacattgagcatctttttaatatgtttacttctcatctatgtatcttctttagtgaggcctttgtttaggtcttctgcccattttaaaaaatgggttcattttcttattgttgaatatcatgagttctttgtctattttgaatacctgccttttgctttatttttgtgttttttattttttttttttattgagacaagttctcactctgttgcccaggctagagtgcagtggcatgaacatggctcactgcagcctcaacttctcccagcctcaagcaatcctcttgcctcagccttccgagtagctgggactataggcacacaccaccatgccctgctaatttaaaagagtttttttttgtagaggtgggatctcgccatgttacccaggtggtcttgaactcctggcctcaggcaatcttccagccctcagcctcccaaagtgctgattataggcctgagccacttagcctagctcagaatttattttttatttgttaattttgaaaaaatataggacctcataaaagtcagtctacatttgtacacattatgtttttggtgaatatgtaaatggattctttgtgaatcaatttggttttgtttttttgcttttaaaaataccagccctgggctggatgcggtggctcacgcctgtaatcctagcactttgggaggctgaggcaggtggatctcctgaggtcaggagtttgagaccagcctggccaacatggtgaaacgctgtctctactaaaaatacaaaaattagctgggcgtggtggcgcatgtcagtaatcccagctacttaggaggctgaggcatgagaatcgattgaacctgggaggcagaggttgcagtgagccgagatcgcgccactgcactccaaccttggcgatagagcaagactctgtctcaagaaaaaaaaaaatgccagcagtggctggctgaggtggctcacacctataatcccagcactttgggaagccaaggcaggtagatctcttgtggtcaggagttcaagaccagcctggcgaacatggcgaaaccccatctctaccaaaaatacaaaaattagctggatgtggtagtgcgcacctgtaatttcagctgctggggaggctgagacatgagaatcacttggaccctggaggcagaggttggagtgagccactgtattccagcctgggtgaaagagggatactctatttaaaaaaaaaaaaaaaaaaagcgggctggatacagtggtgcacacctgtaaccctagcactttgggaggctgaggtgctcagattgcttgagctcaggagtttgagaccagcctagacaacatagtgagacatcgtccctaaagaaaaaaaaaaaataccagcacttagccaaaagatttcaacagtgcagaaaaagaaagttgtgatatcttttctccaaattagtcttgttttcagtttcattatccaagtaaccactactaatagttaaaacatttgaaatacgtgtgggagcttgtcctatttaatataaattatttattgagcaaataatcaccactagtatgttttggatactggaattttcatatgtaggagtccttgaatgtaaggtgcccctttggtagttctgtgcttcttttacctgtactgtaacatagggaaagatgttacaaatggttgtatttttaacagagcagtatctattcttaaacaccagcccttccactaaaggtaaacaacaaatgaatacataaatgaagttttggtattgggattatgtgggttaaacacatccatatttcattattaatatttaagaatataacaaactttttattggcatttggaccttgtagctaaggaaagattaaactttgtttatttgtgctttgttttttttcttcactcagatatttgaggatttcccatttgaggaatacatttattaatcaagctttagtttcaagatccttgatcttagggaataccatcaaccgttcttctttaagcttcctaactttgcccaaatttggttagaactactcaagagtagtttgggtaattcagaaattttattggaaggggaaagaatttttgacccaaattagataaagcaactcttgggtaatgatttcttttcttgttctctctttataatcaattgaaagtagtagtaaggctgggtggcaaaagaaagaggcctggggagaatcgcgtggttttcattatctcttttcatagcagcaaagtgggaagggaccaagaggaaatcaactgaaaaaccatccttctgaaacattggcctaaaaaactgtagtccagaaattgagtgcaactggcagtggcatttaaaaggaatgctctaatttctaggaaagcaggcacgagtacctcttaaaagaagaaaaaaatgaaaactgtaatttaggacacatagacgagtatccattccctgtacttttactctcatgtcctaaccaaggaagggttgccatagcaaatatggcattccttagccatgattcactgttgtaaatgcctgcagcattcataaaagtaagatatatgggctcttctttttccttttaaatctttatttctgtatttaaatctgtatgtcatcatctgtattttctctcttgttttttttaaatcttgggagatggtacaaattatttaggggagggaatgagtttcttgtccacaaatagaggagagagagggctttttgtctttctgctttggaactggagagcttcctatttaggcatggcctttttcaagtgaccttgtattgttatcagtactgtagaaggtaggcacgttgtgtaaactttaaaattgaaagccattaggcattccacttgtaaaccttggctttttaaagaaaattacatgttcattgtgaatattttcttatcgacctatctctgtgcacatgcagacttcctttgcctacattctgaaaggtgtaattgccttctttaaggacagcggacatctatagttcttgggtcaaattgtcctccttctggttttgtcagttctcagccacactgtgtgagcatccattttcttggattctggttcggagctcattttaaggaacatcatgtcccttttgagactctgtggacattggggtgggtagatgtccccctgtgaacagaaggtccctccctaaggaggtgcttctctgtgttgagtcctgcatctgggcacacagagcccgaagcaggaagagttgagtctgaatagggaggcctgtaagcctgactgctctgccacgggtaggcctggtctgccatgctccaggagcccccaggaggctctgaagtcattctgcctctgggaattttacagaggagctaatatttgagctgagtgagaaggtgattcttgaaaaagcaaggcgtgccctggtccagttctgtggggctgtcgtagaagagggctcggaagctcttgggagtgaggctagaaaggtaggcagtgttaccataaggaggttggaagtgacacagataggctaaggaaggggacttcagtaatcatgcagcaatggcctggaaaagggagaggctcatagtagggagaccagtttggaggctgccatagtgttctactgagagagaaaaagacttgaactcagagcacggtcagggaaggtggaggggcagggtctgatttggaagtgttttgttttttggttttgggaacaaggatttggaaacctcttggctccgtagatgaagaaaggtgaaccaaggaagactgaagtttccactcaaggaaacagtaactcagggaagaaaacacaggagtaggaataggtttaagagaatgacaagcagttgtgttttggacttgtttagttcgaggtgacatctttgtagggatgtctagctggtagctgcaagtataggaggaggctggatgtgtgcatgagtttcttggtaaagatccatctgcagtctcctgaagatgtcacccagattgccctgccatcaccccagggccttgagtcactcgcttctcttggtgtctaggcaggcttgaagaaaatcaaacagtacaaaatcatgcaaattcagacctctattccaccccagaggaaagtcctgctagcagtttttgtgtattcttccagaaatttgccttgcaacgcgtttgagtatataaataatcccagaaaggatgggccagacactagcagaaactcaccacacacactgcactgggcatgcagaaactttgcaaaatgtactggtgtgtgctgtcactgcattcctgggtttggaggtcttctgtgtccccagctgtcagtattgcccacaggctgaccactgagctccttccacagccggcccatatcaccctctttcgaagtgtgtctgcaaggctagattattagagtaatttaagtgtttctgctttgtgttttctttaatgaatttttttatgatgaagttcttgttttaaaaatatacagtggtaattaacatgtatgcatttttcttaaaatgacccccccagtccctcttcggatgtgatcactgttaacagtatcgtatatagaccctgttctgtgtggggcgggcagagggctggttagtgggtggaacatgcatactcacaaccatatttttcacatgggaaaatataaaggtgacaacaaatctcctggactataatctcatcaagcagacaataatctctgtttcaaaagtggacaaatacatgctgattttttaaaaaaaattataatagtacagaaagatacaaaaataagttaaagtcttcctaactctcctgcctgtgccctccactgggcaaaagtccctgtccctaaggtaatatctgttaacagttctccttccagaaaacttgcaatgcaaataggaacattttgtgtgtctgcctctgtgtgtatgtgtttacatgtcttttttttttttttttttttttttttttttttttttttttttttttttttgagacagagtcttgctctgttatccaggctggagtgcagtggcatgatctcagcttgctgtgatctctgcctcctgggttcaagtgattctcatgcctcagcctcccgagtagctggaattacaggcgcctgccaccacacccagctaatttttgtatttttagtagagatggggtttcgccatgttggccaggctggtctgaaactcctgacatcaagtgatctacccgcctcagcctcccaaagtgctgggattacaggtgtgagccactgcttctgcccctgcacatgtctaacgcacacaaaagagattctgctgaacacattttttatgctttttctttccaatttaacatatttgacatctttatcagcttatatagctttatttttttaagaggttattggctataaagggaaagtgctttatgggtgcttctgttgttatttaattggatcctgttgatggacattgatatcatttctaaattttttagtatcctaattaatcctgtggtgagcatccttatacagatatccttgttccctcatgaaaatatttctggaggatggaaagaagggaaattttaaaaattatttgtgtaaacattatcgtttgttagtagaatgacctcaggagagattgtagtaatttatacttccccacatgcatatgaatgccttttcaatatattgtttcaaaattggatatcatgaaccttaagagataaatatatatgtatgtattttttttttttttttttttttggtgggggcatgtcctgttattgacagtttgttttggtggagggatgtcctgttattgacagttatcgaatagaactttgtcagttccttcatgtaagggatgaatttggtaataaaacaggtctgacttcagttatggaactgggaaagctggaccttgatgtggaggtgggctcagacatgctgtgtctgggcaggtatctcttgggaagcagtgtcatccctgaacagaaacatggatgagccgcaggggacggtgctgagcagaggggctggccgtgggtcatctgcctcccttggcctagaaggcacaagggagctttccaccttgcctttggttttgagaatgcagaagctactaagcaactcacagtgtgcccagggtggtccagctgaaggatgcgaaatggttcttccttttccagccaaagatcttgaacctcccaccccatgacaagcattataaaattcacataattttgataggctggattccttttctgtagcagatctttcctcagaacagaagtttgttttttttttttaacctaaattacctgtgagttttattttttaaatatggaatctgttttttggacacctccttgtcatattaaatgttctgttattaattttgagattttaatgtaaattttaccccagcaaattaattttgtttctcttgctctctctttttttttttttggcatcctttcccgttgtatagtggtgctcattttatcatactgcttttatatgacttttcttttgtgaacagggatcttgttcacttttccttttttaaaaatttttttatttttttgagacggagtcttgctctgttgcccaggctggagaacagaggcacaatctcagcccactgcaagttttgtcttccgggctcaagcgattctcccaccttagcctcttgagtagctgggattacaggcgtgcaccatcacgcctggctaatttttttgtatttttgtagagatggggtttcactatgtcatccaggctggtctcgaactcctggactcaagcgatccacccgcctcagcctcacaaaatgctagggttacaggcgtgagccactgtgcccgagccacttttccttttaacataaaaacattgatatcctagagagggcactgtattttgggtacatacaggttcagatcacggaagagttgtattctatactttttttcatcattcttagttcagtatgatgtattttataatttcatatgagaaactgtaacaatgggcatgtgtcatccagcaatacctactcataaatcatattaaattttgatccaaacatgggagaaactgaagttttctctgtgtacttggatgctttcagaggcataaaattatattaccatgtgaaagcaagcctacaaaattcctcaggtggtccactctgccactcaaatgagagccagacttacagtgcacactctacaaacggacttccagctcgtcagggtattttaagtgcctgaatatgcaaggcactgtgccagtaaaattactcagtcctgaggatagagcctgttagaattattttaaaatctgtacttgaagtttatttcctcagtattccaagatattttatctggttgttctctgagtatttcacacgtagctagttacatataggtgagatggtgatgcttgtgcctggtgtggatgagaaactgagcctggcagacccgagattggatttcctcttctgactttgcaagtgtggctttgctttaataatagccctcctctttttctgtattcctcttttctccttccagtgtggcaaatatgcatcaatcaaaatacaataacctgtggaaccattttttctaaaataggaaggtggtgccatgggctattgattgttgagggtagtcttcagagcctgtcttataaatctataataattccccccaaattaatgtgctagttagaaccctagaattgcagcactgaaagtgaattttagaatcttttccaactttttaccaagctcagaaagcccttttattggacctttgtttgcctcatggtcatctggtttctttatgctggtagtaacaggaaccttactaacctacaaggtgacccagtccatcctgggcagctgtgatcattggaaagctctgaatcattcatctggaagctgcctgtggcagaatagtacagaacatatgcattgcacacactacaaaactgctttaaataagtgttccttttcattgttagatggaagtgacccagggcaaggatcatgttttatgagccagttttctcctagtgccttgaacataggcacttggtagtgtttagagaatgaattgtcttttttttttttgagacaaagtctcactccattgcccaggctggattgcagtggcacggtgtcagctcactgcagcctccacctcccaagttgaagtgatttttgtgcctcatcctcccgaggagctggaattacaggcatgcgccaccacacccagctaatttttatatttttagtagagatggggttttgcgtgttggccagatggtctccaactcctggcctcaggcgatctgcctgcctcagcctcccaaagtgctgggattacaggcgtgagccactgcgcctggccagagaatgaattcttaataagtttcttatgacttcagattgactggcagttgtgctaaatattttttgcctttatttgaattagaaaattcgtaagggggccttgagagtagatgcagtcacatttaggagatttttcccccccattggttaaaatgcagtttttctatggatctgctttagaactacaaataaactccgatcactcacagcagactacgcaatgagataagaagcagtccatttgttctgtcgtttattcatggccctcatctttgccagccaacctgtgcagaagagcacagcaaagcataagcttcctaacagctattcccattcagctgctgtcttcaagaagcagaagggtttagataaccttagagaacataatcaaacctgggaaactcaaatcaaaacaaaacttcgaaacggaagttgaaggtcctgaggtacccagaggcatagcaaggaaaacgtccccactgggatatcttctggattcacctacagacctctctggagttttaagatccacgttttatgacatattgcatagcaaacaggccgaacgctgttgtaaatcatcctctacgaaaggcccaggttttgaaactgaaggcctgaaaaggctcctgcttaccagctgtgtgctgtacctgacacactgaagcctgagagtgccagtcacctgtcagagggaacacagctgccctcaggcagaacctgagtctagaactcaaggtttttgactcttaggctaaaaataaaaaatcagaaggaggaactatggattgcttaatcagtcacattttcactttctaaggtttcttgtagcaaaagttatagtctttggggattgggatgtagacacttttttttccttccctaaaacaatttttggcctgccagtttttcgagcttcctgctctggaccctgaagttgccatgcagttgcagacatgctcctccttcaaggctctgttaagctgtggctgcccttccttaccactgctgtgccccaggccaaaccccctgcccctccttcccctaaggcactttgcacctgttgctccgagccttctggtctatcccagttggtagagtccctgtgcttacctctgtggctcagtcctcgtgggtggtcagtgctctagggaaggtgagctgaccacttcactttcccttcccagccatagctcttcacagcctcaccaacttagaaaggaatgctgcttttctctctgtcccccgtcctgactatgttcaagtcattgctcagcccagtgagtttgtcctttcttccagaccagtagttttcccccagtccctcaggcttcagcccttgaaggcatccttagccccccaccaccatgatagtcaggcgctgcgcctatccgcagggtgtgggtatgtgtgtgtcttttgtttctggagcccattcctttccttccaccagcctctaggccttcatccaatccttttgcaactcagtgattcatgacttttccctgcatccagtctagtctctttgctggtccattgtttccatagttgccagtttaatcttcttaaaatgctgcatttttgaagtcagtctcttttggtccctctcagaaacatttaaatagctatctgttgccacagggggaaagcttgaacctcttagccagtcattcagagcctgccctctgctggggcctgctttcctctacagatgcttctcccactctgcctgccacagcccttgtagctggctgattgccctgtacctcgcaggctctggaagtcttcagctccttcatggagcctttgccagccattcttttcaagactttgctgttgttcactgcctgatttgttcatttgacacttaattcactgagcaaacattatgaaagatgtactgtttgctactgtgaggaatggatccccaagagataagagggtcagtccctactgccaggaaacttgcctgtgtcacctgggttcagtatcttgtgtctattagatcagaagtggaaaggcagaggccagcccgtatgcttgttttattttatttaaatcaccagcaccccagcaagtgttttgcacaggaaatttctcattatttaattattttgggttttttgattaaattctgtacattcccattttagcttatcttgagttataacattaaaattaaggtagtcatcagctgaattataagacttaattagataagattatttaagatagtgatttctcattagattggccctgttcatattaacttttctgtttttttcttcagtctgcatgaagaaatcagtgatttttatgaatacatgtctccaagacctgaggaggagaagatgcggatggaggtggtgaacaggatcgagagtgtaattaaggagctctggcccagcgctgacgtgagtcccttcctgggtagcttatgcttcggacagtccttgtccacgggctagaagcctatctgctggtatctcatgctagtcctcacatgcaagtagaagtgctctgtagagttgtggtctaattaaattttaaaggcaaacaattttctgcagtctttagaattgaggcttcctaactattttcattggattggatgactaacaactattttttttttgtagtgctaatagcaactactaaaggcaagctatccttagaaattattagtgtaaagagaagaaagacaaatcaaacctcattgttgtagtggtctgttattggatatgatatatcaaaacctcattactacttagttccagcctgccagggtaaacattatataattgtttacagctaaatgaaaatgtcaagtaagaacttttgtcacttgaagttcatttcctttggctaatgcacgcataagtcttttcttatttctttcctgaaattgccatttttcatctctctcagaccagctaattgccttttagacagctcccagtcagtgaacaaaatgattactcaggatttcttcttggcttatttgtcgtttttgttactggtactaagtcttttgttttttgtttttgagatggggtctcactctgttgctgaggctggagtgcaatagtgcgatcacgacttactgcagcctcgatttcctgggctcaggtgatccacctcagcatcccgagtagctgggactgcaggtgcacgccaccacacttagctaatttttgtatttttttgtagagactgagtctcactatgttgcccaggttggtcttgaactcctgggttcaagcaatgtgcccgccttggcctcccaaagtgctaggattacaggtgtgagccaccacacttggcctgttactggtactaagttaatacgtcactttttagggcactttgagggcctgttctacaattttgtgtatgcaaagaagtacacaaaataatactaataaaatccattactttgtgtttgtagcttttcttcaggcactgtcctgggtggtggtgggaagctagggaaggtttttttccaattggcaaaacaaagaagtttcattgtatgtaaaacttgcaagtatatgacagtatgtatatgacactgtaggtaaagggaaaaggcaagggtactaatgttttatgagcaatgaccatacatcgcattctttctcctatgtgatccaaatcagtggttctcagctagtggctattttgttctccagggggcatttggcaatgtccgagacatatttgattgtcctgactgggtacgcactgctagtacctagtgggtagaggccatggatgccatcagccattctatgatgaacagtataggcccttacaacaaagaattatccacccccaaatgccaatgttgagaagccgtgatctaacttaacccttatctttcttaggtggaggttgattatctatctatctctttctctccagccagccaggcagccatcatctgtctacctacagatgaggaacatgagcttgtggttaggttcccaggtccatctcgcctcagaggttgaactggtttcactgtttatcatttttttcccccgagatggagtctcgctctgtcgcccaggctggagtgcagtgacatgatctcagctcactgcagcctttgcctcccaggttcaagaaattttcctgtctcagcctcccaagtagctggggttcaggcgcccgtcaccacacctggctgatttttgtaattttagtagaggtgggatttcatcatgttggccaggctggtcttgaactcctgacatcaggtgacccacctgccttggcctcccaaagtgctgggattacaggcgtgagccactgtgcccggcctatcctttttttattacaattacctgcatacatatttctgcctgagttccaccgttctccatgggttgagatggaatgcatcccagttttatgccacagcatgatgttacctgatgttctttgtggaattgacctaaaggccctcacttgccctacagttaaagtagtctgatcccaatttagtaatctattcgaagactcctgcttagagaacaaaaatgaaggatttgtgattgtgtctctggataatgagggaacattagtgatctgaactgcttctgaaagtttcctgtggttggctttctgtatccacaggtaccacacctccatattaaaccaacaatggattgaaaatattcagaaaaaactataaaaataacaatgtaccaataaaaacaatacaaattattttaaaaatacagtataacaactatttaggtagcatttacatcatacttggtattataggtattataagtaatctaaagatgatgtaaagtacatgggaggactagcataggttgcatgcaaatactctaccattttatagcagggactcaagcatcttcagattttggcatggtgggactggaaccaatctcctgccgataccaagggacaactgtattttggtctatgtgtttcatattgaaccagataagtttaaattatattcagaatgtctgcttgtgaaacagaatccccgcttcatgaagcttggggttagaaaaaaatgctcttgtcataccaaaaagtaccagtagagggtagcaaaaactgacatttctccatatcttggtgactcaatatgataacaacttctgataactcaatataataacaacttctttttctgtttcaggtccagatatttggaagttttaaaactggactttatttacctactaggttagtacactcatgaatctttcaaaggacttttcttagagtgtattcattttggctgtcaaatttgtaaggagtagaaacaaaacaaatttataaaacaaaatggggctgggcatggtggctcatgcctgtaatcctagcacttcgggaggccaaggagggtggatcatttgaggtcaggagttcaagaccagcctggccaacatggtaaaaccccatctctactagaaatacaagaattagccaggcgtagcagtgcgcacctgtaatcccagctactcaggaggctgaggcaggagaattgcttgaacccgggaggtgaaggttgcagtgagccgagatcgcgccactgcactccagcctgggcgacagagcaagactctgtctcaaaaaataagtaagtaaataaataaaggatttaccagcatttaatttgatttaccttgaagtagaatatcacttcacatcatcttaccaagacatagatggtacagagagatggaaaagggatcatgttgcaatggaatcaattagttactaattttagaaattgactgcctggcagagtattgctcagtcccataacttaacccactgacacagatgttaatgtagtatcatgataaaatgtctgattatatatcctcttgaatgtgagttcccgctgtcttgctcactcactcttacactcaccctcgctttcaaattaagaactcattctactagttatggctccagcatcctgatccagaaattcagggtacagatctcttctctgagaaagatcttggcctttcaggactgttgttcagtttcagtcttctcaaatgagacctcttgtgacgcacagccttggaggctctcttttgggaaatgataatgtttctccaaagggtgaatacttgctctctaagaattgaaattgtttgaacattccatcatggttattattattattaccttaaattttaatctctccagaataaagtcagcatcatgtttttccatttgagcttgatttggtatactttaccccaacttaaagtgtgctgaagtggatggaccctggcaatttccgttcttctcatagatgcccttggcctgcaaaagtcataaaatactcaacttcgagttaatatttcttatttaggttgtcagcatcagtaaaacatgaaaaatcacctttcttaaaaaatttaattaaattttatgaataggtagtacattcacatagttcacatttggaaaaggcacaaaaatcaatacagggaaaagtctcagtcccacctctgccccctggttctccttggaacagccgcttttaccagtttctcacatatcctttcagagatatctgtgcatataaaaggacatgtgtgtatattaacacaaatggtagcatgctggatacctgttctgcatccagcttaaagcacttcataatatttctgagttgcaattaatctcatccggataggaaaatcattatgtctagtaccacaagcgtttattaaagaaaatacatgaagtgctttttgtttttttttctttgaggttttacttctttcaaaatcaccctatttcctgaggcctgaattctgtgaaatgactgagaggaggagtttgttaaaatcaacaactactatttcccttctccacaaaaccattatcaccaacacatttagtcttcgttggccagggtgggaaataggttttaattgtactaatgaagtctataagcatgagtgtcagttaaaacaagtttccaacttcttccgaccctcttgtatatgtattctgtgttcagtgacatcgacctagtggtgtttgggaagtgggagaacctacccctctggactctggaagaagctcttcggaaacacaaagtcgcagatgaggattcggtgaaagttttagacaaagcaactgtaagttctgcagcatttcatattaaaatccttagttatttacctatgaaacttgaattaaaattaaagtttggtgagcacagttgcattgcaagtgagtgattctttcattttgttaatgtcaccgtgcttgcacataaaaagttttctggttgtccacactggagtgtgaccatacaatctcggctcactacaacctgtgcctcctcggctcaagtgatccttctacctcagcctcttggggagctgtactacaaacacaacctgccatgcctggctaattttgttgtattttttgtagagacggggtttcaccgtgttacccagactgatctccaactcctgggctcaagtgatccacccacctctgcctcccaaaatgctgggattacaggcgtgagccactgcacctggtctgcatttcttttcacagcagcaaaatatgcaattttattatacacagtactcactgtagagatttttgtttgttttattcattttttttagagagatacagtctcactatgttgcccaggctggtctctaactcctggactcaagtgatcctcccacctcagccttatgtgtatctgggactaaggcgcaccctaccacgccctgcttatttaaaaaatttttttttgtagagatagggtctccctgtgttgcccaagttaggccattttttgaaaagaactgctgatagctcatgtaaataatcctgtcagctttttagaataatttttatattttatcttgtcaggttgttttttgggctatttgcaaaactgaccagtaatgcaagtgggttgtagtgtacaccttaagaatccagcaattttcttattagaaacagtttgatgatacaaaacatttaatacctggcattcctagttcttcatcttatactcagaaagtgttctccaaattattgaggaaggtttttgttcattttaaaattatcattataactatatgtcaactaataaatgagatgatgggcatattaatttatttaactgtagtaatcacttcagtatgtatatcaagataagtatatcaaaacatgtacaccttaaatataaacaataaaaataaataataaaaaattggacaccaaacaaaattctcggttgatagaaattatactgtaatatactgtatgggacccagtgctaaatatgcagcatatagtatttgtagcagaccaggtttactggggtgtgccatatttagaatactcagtgttcttatgctctcatgagatgatggagacctcatgtctagtaggcttccatcccctgattttatcatttaatctggttaaagcatttacattttacctttcttctctttataggtacctattattaaattaacagattcttttactgaagtgaaagttgatatcagctttaatgtacagaatggcgtgagagcagctgacctcatcaaagattttaccaaggtcagagaatttagcgtttatacaacaaaactattagaaacgtaattttaagattctgttgtggtggtgttctaatatttttatatgcatgttgctgtctctctctctctcttttaaatagagctagggtctcactctgtcacctaggctggagtgcagtggctggatcatggcccgctgcagcctcaaactcctgggttcaagtggtcatctcacctcagcctcccaagtagctgggactacagacgtgagccactacacctggctattttttgtattttttttttttttttagtgttggggtctcgctgtgttggccaggctggtctcttaactcctggccttagcctcccaaagcactgggattacaggcatgagccaccatgctcagcctgcaccttacttttgtatgcaacggttttgctttctttgaatctgcttgtaatgatcagtgattaacttataatgtgacctcaagtaagaattaaaagttgagaaagcttttgaagaaattgtctgctctagatccttccttgtagagacagaagagatggaattctactacacagttgattccatctgtttttaaccttcaggagttcagattaagaacctttcctttaacccatttcccatatgccccaagaatactgtgcgggcagtgagctgcactttttttttttcttttttcgaggcagagtctctctctgtcacccgggctggagtgcagtggcacgatcttggttcactgccacctccgcctcccgggttcaagcaattcttctgcctcagtctcatgagtacctgggactcgtgcctgggacgagtaccaggcacctgccaccatgcctggctaatttttgtatttttattagagatggggtttcaccatattggccaggctggtctcgaactcctgaccttgtgatccgcctacctcggcctcccaaagtgcccggattacaggcgtgagccaccgtgcccagctgtacttttttttttcctaaacaggaaataggttaagagttttaagagccttttctagatttcaatccctaaattacctttaaggtgtttcctacaggcttccttacttctgttttgaaattatttaagtttatttctattctgttttcttccaagatagagaataatttgtcaccatcatctgtggaacattttacatacttaggtagttgtcagctttctcacctctaacctaagccattaactcctttggcttctgttagaatattcacaattttttttttctgtagcagctctaaagtttctatttcttcttttttgttttttaagaaaaaaatgttaactacttgatgttacaagcattatcatcttgcataaatgtatggaagacagaaaagcagaaaaataaaagaaagattatccataatcgcactatattgggggtgtgtgtgtgtgtgtctgtctaatattttctttttctggaaaaaacttttaaaaattgaaattcatatgcatgataacattcatcagtataaagagacagtgtaaagtgagtcacccttacaccatagatacttagatcatttttacagaatttctcattgccaattattgttttttgcgttgcttttttatgttctttaaaattataagcaaaaggagtagcacattatacacacattgtcttataacttcataaaaacttaatgttttggaggtttcttccatattagcacatacaggcttgctttattctttttgttggttacacaggcagtagtcttttataaggctgtgactgcttgatttagcagttcttcagtattgttccagttttcttttgctattagaaaaagggattgatgaatatacttccatacatgcatcttgctttacacatgcaaaatgtttgtagaaagattcccaaaagcaaaattttgaggtcgaagggtatatccagataaaattctgttggatatttccctaattatttattctttcatattctcattttctctctctccctcccccttttcttctccctccccctcttcctctatccctccctttcccttttctctctttttctttccctcccttttcttttctcttttctcttcttcctttttcttccttcccctttcccttttccttccttctgcttcctttttttcctttctttcattttttgacaccgagtctcactctgttacccaggctagagcgcagtgatcatggctcactgcagcctcggcttcttgggctcaagtgatcctcccaccttggcctgagtatctgggatcacaggcttgcccaccacacctggctaactttttttttaatttttttttttttgagatggagtctcactctgttgcccaggctggagtgcagtggcgcgatcttggctcactgcaacctccgtctcccaggttcaagcgattctcctgccttagcctcctgagtagctgggattagaggcgcacaccaccacgctcagctaatttttgtatttgtagtagagatgtggttgcaccatgttggccagggtggtctcaaaccctgacctcaggtgatccgccctcctcagcctcccgcagtgctgggattacaggcgtgagccactgagcccggccacttttttattttattttttaagtagagatgaggtcttgctatgtggccacttttttttttctttttttttaagtagagataaggtcttgctatgttgcccaagctgttcttaaactcctgggctcaagcagtcctccttccttgacctcccaaagtgttgggattacaggcatgaaccaccacacctgacccctaattgttctttgaaagggaactgtatctagactgacttaaccaccatgttttgttttgttttttgagacagagtcttgctctgtcactcaggctggagtgcaatggtgcgatcatggctcattgtaccctccgcctcctgagttcaaacgattcttgtgcctcagcctccagaatagctgggactacatatgtgtgccaccacgctgggctaatttttgtatttttagtagagatggggtttctccatgttggccaggctggtcttgaactcctgacctcaagagatccacccgcctcagcctcccagagtgctgggattacagatatgagccaccgtgcccagcccacaatgtttaaaaatacttatttctccatatttttgttctttcctatgcttgcttagtttgatacaatttgcaaaagtataagcttttttttcttttttatagaagccatgcgtgttcattgtaggacatctagaaaacagagataagagtaaagaaaaaaaaatggaaatcaccggccaggtgctatgtttcacacctgtaatcccaacactttgggaggcccagacaggcagatcatttgagcttaggagttcaagaccagcccgggcaatgtggtgaaaccctgtctctacaaaaatacaaaaattagctgggcatggtgggctgaggtcggaggatcacttgagcccaggagctggagattgcaatgagccaagattgtgctactgtactccagcctgggtgacagaatgagggggaaaaaaatggaaatcactagtaattttaccaccctaagtaataatagctgttaagacttctttgaagatgttgtgcctgctttgtttccctccgtggccccagcctatggcatggtttacagaggagtgaatgaatatgtgcacagcaaaaggtggactcattctgtacatacttgcccactcaggtgttctctcgggtagccctgcctcattccctgtgaagcgtggaagggaggggtggtctgtgtgtagtcatcagcccatgtgcaagtcagcaggcaggactcttgtttgccccagggctgtggcagaataatctaaaggtcgctagtctacagtggtacatcaccaagaaaagtgattcttaaaaatctcactgatttagtgctttaagatgttggttactttgtccttgtactctttctattctctgtttacaaatgaatattagagggtcatggtcacaaatgagcatcatcagttacatgctgttagtgtttctatcctatagcaagtacttttttttttttttgagatggagtcttgctctgtcacccaggctggggtgcaatggcacgatctcgcctcactacatcctctgcctcccgggttcaagtgattctcctgcctcagcctcccaagtagctgggattacaggctcccaccaccactcctggctattttttgtatttttagtagagataggcgtttcaccatgttggccaggctggtctcgaactcctgacctcaggtgatctgcccgccttggtctcccaaagtgctaggattacaggcatgagccaccatgcccagccctgtagcaagtacttagatactattattcatttgtacatgtcttacaatttaagtataaggggagaaccattcattacctatagtttacttttttttaatagcttactcttaaaatagaaaattaagtatgttgtatatctctaccaaattttataatgtaaggaccaatttatgcccctcttaatgcttagatctgttgctgatacaggaattcattgaaaatacaattttctttttcagaaatatcctgtattgccatacttggttttagtattgaaacaattcctattgcagagggaccttaatgaagtatttacaggtggaattggttcttatagtctctttttaatggcagtcagtttccttcaggtaagtcatatgggtatagcatgctagtgcacactaaaagcaaaagtgatcaatcagctgggaaacattttggaaaaaatcgaaatcaacctgtaattgcattgctttccttgattacttaacggcttttccctttaaactgggtacattttatcatttagcaaatatgtatttttaaattcctatgaaagaatatttttggttttaaatcccatacattctagtatttttgagacttttcactgcaaattttaacatgcaaaatgtacggcctggtttccataagcataaatagtataaatgccaacaataagaatgtcttctaagcagctaaatcttgtaagtttagttggaattgagaccagctatttgggtaagcgaattagagtcttagtattgtaagtgggtatgtttatgtggcacagggttgccaactgcctgagtctattcgtgagtcagaacgactttgctgatgtgttgggccaagccagccctggttggcagcctggtgcagccgtaaaattcagccttacaaacagtctcccgccattcccgcaccatgggactttagtgttgtgtgtaacaacagtataacctgctgttagcccattatcaactgactgctatgctaaaccaaaattataataatattgcttgtagaagttagaatataatttattccccctctccttgataatttagcaaaaatccaatataatttcttcttttctgcttttagttacatcccagggaagatgcttgcatccccaatacaaactatggtgttctcttaatagaattttttgaattatatggacgacacttcaattatttaaagactggcatccggataaaggatggtggttcatatgtggccaaagatgaagtacagaaaaatatgctagatggctacaggccatcaatgctttatatcgaagatcctttacaaccaggtattgaaattaggtaaatttgtgggcattcaaagagagggcactgtcagtcaccttattatactttaaattctctttagatgaaaaatgaaggaacaacttctaattgttattcttttttcatcgaaatatttcatgagcaaacatactaaaataaacagacacagacaatagaaaaacaccttggagacttccagataagtagggagtagaatctgtttaaccctaaaagcatagtagaaaaggcattcacttatttggatgggttcatgtttggtggctgtttctccttcttgggtccttattgccttgattacaaccaattgtcagcaattaatgaggctttaatgagatgattctgaagtcctgagaggcagcaagcatagtaatatatctttgaattcatgagcagaagggtgcaaggagacaatgtattttctttttgaatttctcctttcctgtttgattttgcatgtctctttgtgctttttccagcttcatgtgggcttgaaagtaagcagaaagtaaattccttccatgcttttctgaagttctgtttgcttgcttgtgtcctgatttttgtgagcaatattttttcttgatataattgtaaaatagattctgcgttattggacttcagtggaagtgcttttagtcatttgctttaatgtgtaaactttgaaaatgagtaaggaaagggggtgaagagatagagtagttgcctaggaaccattttctggcttattgagctgccttataaacattaatagttctatgtgtttattcattgaggaaacattacattgattgggagcctgctctgttcaaaagtattgggccaaaggacacgaagacttttcagcaagacgatccttgctttttaggggctcataatttagagtgagaaatagatatatagctaatataaacccaaaaaatatagaagtatttctgatgtaacttggggtttcactcttaggagtgaacagggcactatttcttttgtttgcataactgtttatgtatggaatgggataattcttgatgggccagaatacattccggcaactgatacaccataatgaagtaccaactgcatgattcacatattcagagactggggagctttggggacagctcacagctcagcttccaggcacaactctggtgggataactatggcccttgctctcctggaagagagtcatcaacatttagtgcatattaagcacagtcaggcttactatgttacgtatatttcttttaaaggtaacgatgttggaaggagttcatatggggccatgcaagtgaagcaggcctttgattatgcctacgttgttttgagtcatgctgtatcaccaatagcaaagtactatcccaacaatgaaacagaaaggtaaaagttcatctataaccagcccattgtgtcaaaattagttgtggcttcttatcttcaaattaatgttattccctccctctccctttctttttaaacacatgcagcatactaggtagaataattagagtaacagatgaagttgccacatatagagattggatatcaaagcagtggggcttgaagaatagacctgagccttcatgcaatggtaagatattttccttggtcgattgactgagtattagaggcttttctgtgttgtgtgcgtttaatgggaagaaacgttttccaatcttttgccactctttcaggaaatggtgttaccttgatagtagatactcagcagttagataaatgtaataataatctatctgaagaaaatgaagcccttggaaaatgtagaagtaaaacctcggaatctcttagtaaacactcttcaaactcttcatcaggtccagtgtcgtcctcttctgccacacagtccagctctagtgatgtagtaagtatgaaagcctcggctcttctgaactcagatgcatgcacgttctcttgctggggttaacactgtctcgaaggctaaggctacttcctttgcttacatgttactgggatattttaataactttcatgcttgtacattttctcaacattttgttatgaaaaagttcaagcatatagtaaaagtgaacaaattttagtgagcattcatgtactcaccagtagattctgctattaaccttttacttgcttatgtcatacctgtctatccatcactctatccattaattcatcttattctttgatccatttcaaagtagattacagacatcagttcccctagagtactgtagcttgtgcatccttgtagccagactccagtatttgtttattgttttttccttttttttttttttgagacgggatctccctctgtcacccaggctggagtgcagtggtatgatctcggctcactgcaacctccgcctcccatgttcaaacgattctcctgcctcaacctcctgagtagctgggattacaggtgcgtaccaccatacccagctaattttttgtatttttagtagagacggggtttcaccatgttggtcaggctggtcctgaactcctgatcttgtgatccacccgcctcaacctcctaaattgctgggattacaggcatgagccaccacacctggcctttaacgtttttcttttcttttcttttcttttttttgagacggagtcttgctctgtcacccaggctggaatgtaatggcatgatcttcactcacctcaaccttcgcctcctgggttcaagcgattctcctgcctcagcctcctgagtatctaggattacaggcatgtgccaccacacccagctaattttttgtatttttagtagagatggggtttcaccatgttggccaggctggtctcgaattcctgacctcaagagatccacccgcctcagcctcccaaagtgctgggattacaggcgtgagcccagctgttatgactttttaacaccatagttagttttgcctgtttcagaatttcatacaaatggaaccacatagaatatagtcttgtgtaaggcttctttcactcaattttttttcagctttctggttgaattttttgttgttgttgttttgttttgttttttgagacggagtctcgctctgtcgcccaggctggagtgcagtggcgcgatcttggctcactgcaagctccgcctcccgggttcatgccattctgcctcagcctcccaagtagctgggactacaggtgcccgccaccacacccggctaattttttgtatttttagtagagccagggtttcaccgtggtcttgatcgcctgacctcatgatccgactgcctcggcctcccaaagtgctgggattacaggcgtgagccaccacgcctggccaaatttttttttagttgagatatagttaacataaaattcagcattaaaaatgtacaattcagtggtttttagaacatattcacaatgttgtgcagccatctccagtaattctagaacatttccatcaccccaagaagaaaccctgcatttagcagtagtttcttctaattcttccttccctcccttaacctctggtaacctctaatctactttctctttctatcctgatagaattttttttgttcccccatcctgatagaatttatgtgtcaattataatgtaagttaccttttaaaatcaaagtgaatttgtagtgtactgatttgagatctaaagcaggcttacctgtttgagtttaactttattaagtgtaggacatgaaaagtaatctaaatattgtatgttgttgatgatgaccatgtgtcaatatggaatcataaatcctcctgtgcagaatctccctgtgtgcttttttggttcctagagcagtatgctttggaggacagaagccaagctagatgtcacagacacagggagatggagtgttggggactgagagaatgtgactctgacatgctgggtagagtgccagggccagggtggagacctgcagagagacgtagcattgtcatggcccatgcagcccagaaataggtggagctcagcccactgtcgcgggaagtccacccccacccacaccagtatgtttggttagaatgatcactgatttgtcatcacagactctcagagattgaaccccttaaacccatcatcttgtgtctggctgaagccagggactaggacctaggttcctcactctttaccatactctttcattttctataaataaaaaaacaaataaacttagacctctgtgagctccttcaaggtcagatgtgggcagtggatttaacaatgaacagaagctctctgataaaatcagtcattttaaatgtttggaggaaaatttaaacaaacagttaattttttgtgtgcttcttttaactcccaaatgtttaaatttagtccagagagtactttaaccaaaattgtttttctttctgaatattgagtatctaaattactaatatgtcacattataactcacgtgacttgtgttaggattccgatgcaacaccatgcaaaaccccgaaacagctgctttgccgtccgtccactgggaaccgagtagggtcgcaagatgtatccttggagtcctctcaggcagttgggaaaatgcaaagcacccaaaccactaacacatccaacagcaccaacaaatctcaggtgtgtggaacgtgggtttttaattgttagtatttgatacaaaatatttagaatttcccacatgtaaataatatgcagcatgggtttgaagaaaacgctagattgaagaacaaacttattttattctaagaggttccaacacatgacagtgcttctaggaacaggatgtcctaaggatccttgtgagacaccattgtaacataaatctcttcaggaatctattgactggtccttataagatgttccagccaaactaccatataaaaagtgtctcagttgtacatgaaataagctggcatgaaggttttgtgaggcctcatggcagtgtgcatatctgggaataatgtatccttttctaatattttaatgttcaataccttgttgctggtgttgaaatgatcagctggctgtcaggcgtggtcagttgattaacattagcttggacttaaaaggccacagagatactctagtttaagtttttttgttgcctagaattgtcattaactgagtaatgactcagagtgaggggaggaagccattgatatggggctctggcctaaggctgggtcactcctcactatagctgggaacctgggaataggcctcttggctgtaaccttgtgtctgatttgactcactgagttcactttacctacgcggccttagccatgtatgccagacacagacttatcacaaaataccagttcaagtgacagggttgacagaagggactgaggtcacgagaaagccacaggccatgagtagcaggaagggagtagccacctgtgctgacccaaacctaccagtgtgcctgtcatgcccaagagcctcttcccgctctgtcacttatgatgtggtgtttgggttggtaagtttcttaagaaaacctttgcatcccacacactgttaagaaggcgggcgagtggtctatgcttttcattatttccattaaaataaagtaagggttttagagctaaaaagaacccctgtaacagctttttctgcccactagataagtcagtgatcagtaaggtaacaatctagccagcatattcctagttttgaaagcttccccaaatccaggtgtttgagaacactctgcttttctgcaatactgatgtctttgtggtcgttttctgtttctgcagcatggatcagcaaggctctttcgttcttccagcaaaggcttccaaggtacaactcaaacaagccatggttccttgatgacaaacaaacaacatcaaggcaaatccaataatcagtattaccatggcaaaaagaggaaacacaagagggacgcgcccctctcagacctctgtagatagtcagcgctgcgcggtggactgtcttctctgtgcaatgatctcatgctcaggacagttgcgcagggactcctgggagatattcaggagcctcacactgttcagacgttgacttagcaactgcgttttttcccagctcgccacagaatggatcatgaagactgacaactgcaaaaaaaacaaaacaaaacaaaaaaaaaagcaagcaaaaaagagggaaaaaaaaggctgcttatttgataagtcatatgctacaacagggtcattttaagatttaaagcttgaatgtaaaataaatatatttctcattggctttatgcagagttatagggaatagtattcagtgttggtagggtgatagaaacaaaaaacagtatcagaggatgaggtggggaaggaaaacaaaggtatctgataggaagtccagattccaaaggggaaagtgatctgtgcatgttttttttttaaatatttttgcatatatttaccattttattgtgtgtatatatagaagaccatataggagattgatatttgtaatagtggatttgttaataatactttttacataacattactgtttaaattgtaaacagattttttctcaggattagtttgaaaaataatctaaattgtcatcttaacatccatatatagggaagtgattagttctattactcaatttgtttttctcagcattgaaatgacttaatagaacccttgtgtcctgctgcaaaaatttttcctctctaaagaaaaggtttatggtggcaaatgatgtttattttattttgtaaaaaaaaaaaaatgtactatgtacttttgtgtaaacactgaaaaatctctggtcatctccgagaattaacttgcaactgttttctatagtgctgtcgtcttgggcaatgggcaattacatgactttgtgtttgcttcctttgcagtcttttttttttccccccatttcttcctaataggaaaaaaaaaaaaaaaaaggtcacccatgtctggtctcattcctgttgcagtgaaacttcgagttccacagactttgcatgctggcttctctaaccctgtgtgctgcgtgtgcctgtttctcatctcttattctttttaaaattcatgcttaactactgtgggagaataactgtaaacagctttaattaaatcatacttataaaaaactattttcttatattccactctatgcttttggtattgttgatctttacaaattaaatggtctttgataatggatctattttgtattgccttattaagaccaaatacttcttgtcatcccattctttatcctcttctttcatggaattgttatcgttaattaaaacttttttaaacattggcttgtttcaatcatactgtaaattttggttgtagtcagctttgagtgcaatgagatgtataattctgttatcattacctgttgagtttgaaactcagttgggaatatttaatataatagaatgtaagtgacatttctgaaaatgctttctttcagggtgaaagctcttatgtttagcatcaatgtgtatggctctgttaaatgcagccatttctgagacgagattcttttatatatatatacatataaagtactattggcttttaggagtttcttttatatacatttatgaaatactgaagaccaatcagaccattaatggacacttagtgtaactttttataaagaaaataatgctaaagtaagaccaaaactgatgtcatcactgaaattaacaattttcaatatgttcatattttaattcacaatggaaaaatgtgttccaaaactggaaactcatagtactcgtgtaaactgtggaagatttcaaatgtgatgttattttgacaatgttttaaattttagagtcacattttattctgatcagaatttttattgagatgttgagcttttgtttttgaaactagtttgtcataacattgtgcataatcacagtatttattttctaggacaattgtgaatgtgtagacttatgtttactgctaagggaacaattatttataaaataatattaaatccagtattagctgcctatttcagacacttaatacttgcagagatctatgttacatttaccacactgaagttttttttgttgttttttgtttgtttttaaagaatcaccctcattgttgaaagtaaatgtactcttagggtgcgaatattagtgttccaataagcatgtgattatattaaggtggtggtagcgggaagataattctgattccattgggaatcttaggttttcgtaaatttattgggaaaatagtttttcctgtactgctgaagtttctttttggtaaacagtatctttctaaaagaaaaaagcatgaaggagaaattgaggtgtgtatacatttcctcaaatgaccagcattgtattcgtgaatactgtgtatcttgcagtgaacagtgtggaagctgttcatttttcaatctgaagtaaaatactttcaagaacttttagtttgcctgctcatttgttttatacatttcatctatttgactcctatcttatttcttttttgagttttaatacttcctatattttgtgaatatatcagaaatgtgtcatttatatattagagtccattcatatccatgaatcataaccttcctttgctaatacttgttgaatgggattttacaaattctccctcactctggtgacatttctcaggcagtcatgtatgtgtacctggccattagaaatattaatatttaaagactgttttttagaggagctgatgggttggtgaggtgtcagcacaaaatcttactggttatgttttgatgataaaagtatatccattttttccctccagctttaaggtgactgtgaaggtgcctggttttgaatgtctttgtttggtttggagatgtcgcactcagttttcaaatctagcttggatctgtaggacctatgttttttacaagtaattgccctccagtcttcaacagttgattctgttttatttttatcctgttttgagtgtactttacctttacttgcattttgagcctcattaatatttaggttatttgatttggctccagatattcctagatctgcacagggcaaaacatgggctatagggtgagcatttttaattgtctttttctgctggaaccttatatctctccatgtgttttctgctccttccctcccccatgaaatggtaagtgtgacttgtgtttgcctgaacctgtggactagtgtttggggtttctggaaacactagagggtcagaaaagagtaatgaccaccgtgacgtgcaggattctcttgctgtgacatgttcattgcaaagccctctccagtgactaggaggtgtagttattaaggttgatctgttagaaatcaccattattaggtattagtggtagatgttgctgatacttttattggtcatgactacatctcagttttactttaatattgatctatagtttgatcagttccttgaattctaatatgttgatttctcagtgtttctgtcactaaccaagaatgtttctaggcagttggttgcttcacagtcaaaactaaatggtaaactatcaaaaatacattcccaattttgctgtgataaatattgaaatgttaaaattaatgaacagaagaatttattcttacccatctattcttgttctcctagttcattaaactttcagttattggaaaggcacattctcaaagtattttatgagcaaaatattctataaatgcgtctaacaaacctaattgaatataaaagttatatttagtagttactgttgatagtaattttcatcagggtcatagttcatctagtaaaatatttagagaatgatgttaacattccagcattaaagtgggaacaaagatttatatatgaaattccttaaaagagttcatcttgccttggtttctgaccctcaagactctagctacctgccatcttgtcaaaacatttgtgggtagaataagtgttaaagatcaaattttaatatgcttctcgatatttaacatagctaagaagccagattttactgtagaagttatttacatgatttgaaaacttgacctaactggaagcctttttctcagtcatcttgttctaagccatcttgacttcacacccttagcgacttttcttttttttttggtcaaagataatgagctaaatatatatagacgttgaatgttgacaaaattattaaccagaaaaattgcttataaaggctgctgatctatttgatacctagaattaaatatttgaggacagtttttagttaataaactgctaatgtttattttactgtctctcaggtttttggtttttttaaaaaaaatgtgtttggcctttacattttctacttaagtgtgtactttattgagtttaaccttgtctgtagcctagtagcctgaaagaaaaggagacagaaccagagagatggatgtagtgcattccctttggttattacacatttgtggtagctcctggatttactgagagatattttagctatgtcaataagaacagctaatgatgtggaaatcaggtgttctcttgtgtatttcagtgaacatttttattagtagttgcatatcatctctagttccacattttaacttaacgtctttgtggcttcaccactgagctacctttcactacaccagcttctgtgtggcctggtaacatggaaggtctctcctaaggacagtctggacgtattttgggggaatgttatttatcttaaagatgcctagaaacaaaacgcatatagtaccagtgagaaactatgaagtaaacaagttgctcaggccgggcatggtggctcacgcctgtaatcccagcactttgggaggccgaagcgggaggatggcttgaggctgggagtttgagaccttcatctcttaaaaaaacaaacaaaaacctgaatggtgaggtgtggtggaattgggtaggggagggaaaggaggacttggaaaagcattctccaaagccagcaacttggtgaagttcagtacttgcctcttagaggttaggccatgcctttcaaagagagtgaaatgatgggttatcagccacattcttggagttaatatttttcttcatctttcagtttgggttctgtgctattcatagttcttccctaagaccatttcattattaccttttatatttagttgcaatttattataatatgttgttttgtccctgaacttaatctcctaattttaagatcctctctgatttttgcatattgaaacttacagaagtcactttaaaaaagtcttttgaaagtcctacaatcctaaaataaatcacaagcttgtttgttagacgtgtcaagagtctccagtctttactactaaaaagcagcactgccttaacacacattgttatgggtgaaaagtgagggacgaccagtgtagtttctggatataaagtgtgaaggactgttgagttaaacatttttagtggaatatacatagataacgtgtatttagaaactttggtgaagccagtatttgtttttagtaacctttttatgtatttccttctttgattagcattgtcttcagtgttaagaaatgtggactcctgtgaggtgctggaggtttgaatcatcttgaaaactttccaatcttgtctagttaccactgcagagacactaaggaatttaccagaaaaagatatttgatacaagtgatttaagaaatctcaacatttcctgaggccgtatcactgggcaaccagtgatgaaaactatgaatgaattgcacacctggaagattttttaagctaatgacagtttcttcaaagatgtcaattatttgccttggaaattttataaattgcatttctatgcacatcggcctctagtgcttaccactcggtttattattcataatctgcaattcaataaaggctttgtgttttcatttatcttcaaaa

SEQ ID NO: 2 - DNA, 44042 bp, Homo sapiens
  source 1..44042: mol_type = unassigned DNA
ccacgctgccctgcgcacgcgcagcgcggctcaggcggcagcccggtgacaccggccctagcgcgcatgtctgcacgccggggtctgcgcgccgcggcgggccgagggcggcgtgcgggccttccagccgctgcctattccacccaacgccggcctagtcagtaatggctcaacgccaccgcctctccctccagcccctcccagtcgcgtagcttctgacgccgtcctcaccccgcccgcggccgacggggccccaacgcgcaggcgcggtaaccacggcggcagtgtctagtgaggatttgaaatcggtcgcgcgtgcgcaccggcgacacggcccggcgaccgaggccgcgcttcctcctgccgcccccgtccccgccccctcccccgcccctcattggagcggacgcggcggcggccccctccttcccccgcgctgtcgccgccgagagtgtcttttcaccgccgccgccgccgccgccgcaggagcgccgagccagcggcgcgagcgtgactgagggctagccgcacgggcggcggcgcctcccgcgggtccttcagccgctcggcgcctgggcccgccccctcggccccgccgcccgcccttctccgatggcctctccccgcggcccgagtggaacgccgccgccgccgcggcccccgcgcccgccccgcgccgcgtgaagcgggagcccggagaccgcagccgcccgctgggacgcgccaagcgccggagccgcccgccgcggcctgccggggcccatcaccgccgccgccgccccacgccggagcccgacgggagcgcggctagagcaggaggccggggctcggcccgcccgccgccgccgccgccgccgccgccaccggcccaggcccgtccgtccgtccgtgcgcgcgcggccgggcctcggggcgcggcgggggcggggccgcgtcggggcgggcgggcgcgcgggccccgcgggggcggcgcgtggatggatccgcgcgtggcctggatccagcccgagcagaaggggccggccaatgccctgtggatgcagatctgggagacctcgcagggcgtgggccgcggcggctcgggcttcgcgtcctatttctgcctcaactcgccggcgctggacacggcggccgcggcgggggcggccgggcggggcagtggcggcctgggccccgcgctgcccgccgcgtcgcccccgccgcccggccccaccgcgcccgccgcgctgccccccgcgctgctgacggcgctggggcccgcggccgagggcgcgcggcgcttgcacaagtcgccgtcgctgtcgtcctcgtcgtcgtcctcctcgtccaacgcggagtcgggcaccgagagccccggctgctcgtcgtcgtcctccagcagcgcctcgctgggccggccgggcggcggccgcggcggcgccttcttcaacttcgccgacggcgcgcccagcgcccctggcacagccaacgggcaccccgggccgcgcggccccgcgcccgccggctccccgtcgcagcaccagttccacccgggtcgccggaaacgcgagaacaaggccagcacctacggcctcaactacctgctgtccggcagccgcgcggccgctctcagcggagggggcggccccggggcccaggcgccgcggcccggcaccccgtggaagagccgcgcgtacagcccgggcatccaggggtgagtgcgcggggaggccgcgggggcgggggcggggcccatggtcctggccggcgcccgcggtgcagacacccgtcccaggcgcccgggcttttggaggatggatgttgaaggctaaggccaaggcccgactctgcactgaaagttttttttttaaacatcagactcatttatcgtggagtgacttgcccagatcctacaagtaacagtccaagaaaaggggctgctgggtaggacctgcaggtatttgtcttttttactcttgagattggaacgggaaatcgactctctacccctccaccccgcctccgggcaagtgaggaaccccttgtcaaagtggggcgtagataagtgtggagtttcacgtaagttaagttgcagaataatttagcattgccaggaactcgaatcacgtcgaaggtaaatattaacctttttaatttcattttttaaaaaaatttaactgtcaacttagaggtgattcattttttggggggtgttgtgtcctttaattttgtgctgcaattaccataagcatcgcctatggtttataaacattggcttaattcaaagaaaaaaccagatttgtcatatatgtctattctttggaaggtgccatttttattttaaatatttctacatccgcctagagggaattagaggctctacttaaatttagtgcacttacagacggcaaggaatgaaacgaaaggtggtgtgtgtttcggggttggaattgtcccaggtgaggctgttcaggtgtgatgctgttgacgcagcccctttgccattttgggcttttctgagcgtctggaagcaatttatgtgtaggttgtatgtcagtattttaagacttaaatgataatttttccttgcacaatttttccccccaatttaaaaaacaattaaggatttgctggggtatgagggttgttgcatgcagtagagtcctacaaataaccacaattgctaggtgttgggagttcttatagtaaacttttgcttgtaactctttttctcatttgaagtatgttgggaaacacgagttgatacttctttaaagcgtgtgatacactgtaatagctgcatgttctgtaacttatttcacctggcttgggctacaagcgataccttctaaatttcccgaagtgtaaacatgcagtgcagacgccggcaggagcgcagactcctcttctctccctccagttcttacctgtagaactttctgagagcaggtggttgggagcagtttcttcttgatgaatagcaatatatacctaagggctcgcttggggaggacctttaggtttccagcctgttatgtaactggaatggactccgtttctcttagcactagaaaaaacaggaagacacgtggtctctgccagtcttgggttgtacctctgctcttagaaagtggtagcgcatcaggtcccatgcacctcacttgggctccccgagctgtttcctccaggtaactctagtcaggctcagtaggtggtgttgcttttgttagtgaatgagcccactaggatcaggtgctgtgctaggatctggggatctggggcagggacggcatgggagacacagtctcatgatcttgacccaccctgaaacctgcctgcaagtgccccctgcctagagcacaagagtaccctgctcagttgtgcaggtcccctctcacgctcttcgtcacccacgtccagtctctcatcacatgctgttcgtttggcccttttaatctctccacacctcatcctccctcatgcaggctcttgttacttctgtggagattgctcaagcagccggaggtgcctttgatgcactctggtcctgcttgagtcctttcccatcacaggtaatggggcaatcttctctgccagcaggtctggtcagattccccttcaccctgtgatgtcacctcccccatctccaatcccccacattaaagactaaagcctaaaactcagcaacacctccagggctctgctgctcagatgcccctaggtcctggctcctggccccaccccttgccaacccgttagaacctagctgtaaggcagtatggtgtagggcatgagagggctctgaagcccaccaggaggtctggtttgatactgcagtcatgctcttggaactttccttttcccctttgctggagacctctccttgcctgtctctgggtggtgtgtaccatgcctccagtatggccttgggtgcatccgcttcctgccgtctctgcccaaaggggcctgtgaggaccacctgctctgtgcccagaagggcacggtgacctctgcttgggctgctattccagctactctttcagaagcaaacctaagctgtggtagagttgggcctgtggctagaggaggtaaggtcccccctgtgatcatttccacatggcctgttggtcctatataaactaacctttttgtatcaataaatagttctgtctagaacttgcttctggccatcagcttacccagagtgttgagaaaggccaccaaaaagtctttcggttgtggcttagctaaggaaataactgagttttaaaggctcacctgggctggccaatagtaaaggaccttgttgctgagaagctgcttggggttgtgatatagtcccaggacatgcacttctgaaaatgcagtgtgtattcctcatgggaggatgagcctgctgtggagcattggctgaacccagttgggtctttgcctggtagcccatgtggcaaccctcactgtttgtccttttctggggaggagttttctgcccttggacactttgcctggtggcttggccttgtgagactgccagtctgccttctgcttcaagtaggatgaagaaaaagcaggtgaaagaggacagggattggtgcaagaaccttcagaggagaggaggtgaaatgcttcttttggctctggttcttgaatcattgtttgattgaaatctcaagcctttgttttgggagtggtgtgcttagcgtgagctgtgctatctacctgggcttctgacctagagatgttgggcaggtgtttggacaggccctgggccctttgcatcccagcctctggctgctgtcacttgcaagtgctctcatcccctgacccagcagggctttgggctgttgttactttgtcatggtcgttctagcagctttggaaacctcttcaggttaagagtcttgcataagtgagagtgggagcatggccctcagatatttggccacatccttactggtgtgttacagagacccaggaagaatgtagttgaacgaggacacatgcaggtgtcccgggccctgaacagcttctattcagagtttggcctttcgaaggctgtgccatctcaggtgtgcctgcagtgtgcagcaggtgtatgcagcttcctctttgcaggtttcttggtatttaatctcatcctttaatatcttctattaactcaaaggaaattctgttcttagtttgaagtctgagagagaccgacgactgtcggcataggacatggtcagccatgccccgcaaggcgtctggtgagagtcgtttccaacttggtgcatgtttttctcaattctttcttgcgaaggagtcacagcttggaggcgcaaccaggatcccccctctccctctagcctgacctcactgacataaagtagagcaggtgtgacctgtccggaacatccttgtgatgctcagcagggcctgctgcagagcacgggaggcatcactctaggggcctttccctcccatactttcctgtgagtgtccaggatgcatgagagaggttgttgtgagacctgccttaaagggtggcggtggcacgtgtggaccttgctttctgagtttcactctccgagtcccagaggtataagcttgtgaagagaagcgtgtatgtatgatcacactaagcagatacttgctcctgcactgttggaggaggaagaggagttaatttcatcaattaactcttccaacactccttccatttagtaatagcatcacttgttcctgtcttttgttccatggccaagctccacaaggtgaaattgaaaatcgagtgcaagacactggctgtgctggagttgaggaaagttttgctggagacctgcttgcaccatatgtctggtcactgatagatgaggactggccaggtcaggacagctgacacttggagaaggggctgcccaggagggcatgacagactctggaaaaggagggtcggagtattaaactggctgggaatgagaggcctccaatcttttcgcaggaaaaaaaaaaaaaggcttaatgctcgtgctgtggaagtcagaatggagcaaagtgggttctgtctgtcttgctgctgtgagcgtgtgatggaacaacagtgtcatttgctttttctcagaaatatttaatgcatgtttgtgacataatttttcaaagtaattttaagtaaatattttaaagtaaaaagttctaagatttgtgtctcaaggtaaagtctcaaacgttctttggtcactatttaatatgagattttgtcctcattttaaatggattcatgtaagcgtcctgtggaagaagagttaatagttatccttggaaaataagaactttttatgcctcagttaggtcatatggtttaggatctgattttgtagttgtggagtaaaggttaagaaaaaaaaacagcaaaccttgatattcaaattcagaaacttgatttttgaggatgcaaccagaatttggactaaaggtacagaggggtggcagagtcagaccacccagacttgcagaagattgaagaagccgcagtgctgccatgaagaggccctttctaggaggtgtggctggcttgtcagtgctttgtcttctctgcagtgaattggatggcaagcctcgccctcttcgaaagctgccactctgaacctgccttgagaagcacctcagggagggcaggcaggtggtctcagtcagcgctgacaggtgtccaagttacctgacctgttgggaacatgtgcctgagtgaggtggccaggatgccttttctcccaacactggggatgcacactcgtcagcatcctatttttgagatttctatgttgtggtagttctctgttgctgtgtagcaaattaacataaactcagaggcttagaagaacactcacttatggtcttaaggattttgtgtgtcagatcagggatggcggggctgggttctctgcttaggcttttgcaaggctgaagtcaaggtgttggccagctgtgttctcagctggcactcagggtcctcttaccagcacattcctgttattgtcagaattcagttccttgcaggatggaagtccttgattgcttgctagctgccagcaggggaattgggtagggcgctgtcagcttcttaaggccacctgcattccatctgcaaagcaaggtactttgaatttctctgatgttttccgccagctggaggaagccccctgtttctatgggcttgtgttattgagtcaggctcctgcagataacctgcctacctgaaggtcatgtagtagtacaacatgatcatggtgtgataacctcatcagagccacaggttccaaggagtagggtgtaggaccttgaggggaggaggttcttctgtgggtagacttcccctggcatagaatccgttgatgagcaggttgtggttcttcttgtccaacacttttcccctgactggactccagcccatcgcaatgactcttgcagattgccagttccgtcctctggcttggtggttactactgaactcaggcagccactataaccaggagaacctttctgtgctgcactcagatgaacattctttaaaatatgtcatttaagaaaagtttgcaggactactcgggaggcagaggcaggagaatcctttgaactggagagttggaggttgcagtgagccgagatcgcaccacagcactctagcctggtgacagagcgagactctgtctcaaaataaataaataaataaataaataaataaataaataaataaataaaagtttgcaggaaagccgtgtgaatatatgaaaatacagtgattgaaaagtcctgttcacgaggtgtcctgcattggctagtttaggaaaggggtctttcctatgcaggtgggggttgatgacttacccaagagtcacctctggaacccagttctcttaagttgatagcagtctatctttgctttgcagaagatgtggggaacacttgtcctgcaagcccaggttcgtagcaatgttggcttccccagaaccttggcttcagagcactgtgcctcctttaggaggcacaagaaaactcccacacggttcttccctctgtcccttcctgagccccctgagtgtttggctcttgtggagttgctgctattactaagttgatcccactgccctcctgagtctcctcagggaaggaggggctgtattgtagcccgcattcttagtgccaagcacacggtagccactaagtaagtatctcccaagaaagaagagcaggaggaggatctgccaactcaggagagcaggtgggggatggcaagtcttcggagtattcataaaccaaatgctaagggaaacttttgttgtttgtcttagaattttaaaaaataaagtctgttgcagtttatctgctttccttcctggagagtggctaaactagtgttctgttttacaatgtagaatgcaaaagcagaaaacattcaagaaaattctatactgtatttgaaaaacatcaccatttagttttaactgctctttgtttcttattataaaaattaatacctaactatgaaaagttagaaagcctggagaagtatggaggtgagttgtccaccattgggccactagagagtgccctatgagcctgtccctggtgtcctggactctggtggtgtgcactgcatttgctggcagtgagccagggggtgggccacatctgggcccgggcggggtggatctctgcagaagtttatccatctcttggctgacagggtgggcgagatgggagcagctctgagggtccctgttggcagagaatgtctttgattatcaacaacatgcctttttgttgtgggcttgtgatccttttctttcctaaatcagctgccgtgcataaccagttaggctctcctgtggcttcagattggagttagtttcccaagtgctaggatgtgggtgttaggtgatttctgtctttccgtttgaaagagatttcagatcattgtaacatttctggaatcctgtcgatctgaaggaatggctaggatgtagtagtttaagggaaatgaaaagtcgaatgtattttgatgtttctgcatcagacctgctcggtggagtccatttctcagcttcgggagccacgtgcttggctcttgagagcctagctccatcagcccatgtcacacactcacaggtctggctttagctggtttcgccatggtttctaacttgagcctcagtttccccccctgtgaagcagagcctgtggcacccacctcagagagtacatgaaagacttggaagcactctgtgagttgtcatgcgagagattaaaaaggccaccgctgcccttttctcctctctttaaggaaattgaaaccaaaaattaagtccttcttgccagctggacaggaaaagcctttttcttggtttttgaaaatacaacttccactttcagaccaaagtgaaaactgctaaagactgaatattctgagtcttgggagtggggggctagaggggtgttgtgaattgaaagatacctttctatttttaaaacattttaacaatgccttaatgatgaataatgtctgttctagttttgcatttgttagtttttttttttttttttttttaactgttctgaaggtacatcagcactgttctacagctttaaataagaatctcatctccccagaggcaagggtactcttgatgtatttgctcagggctgtatgtgctgctccgtgtaactcatttaaagttggttaaggtttttttatttcttgcacatagtagaaggagtggatgaagtgttttctgaactctttgcagcttctaacatagtgttctgtgtatagtgaaggaaaacaaaattaagggccagggaacattaagtaggcaactagaacagcactgtccagtagaacttcctgtggagatggaataattctatttttgcactaatacagtagccactggccatatgtgacttttgagcacttgaaatgtgacaaatgcaactgaggagctgaattttaattttatttacttttaattaaaatttaaatggccatgtatttagacagctctgaactgcagtaactttaggctctatttgaaacagtgtttgattcagtaactgttgctgaaataaattgaaactcatacaacagtaaaagctgtgttactcagcaagttatcactgtgaaagctctagaaattgtttgagtttccaatgcaaatccttttcaaaaagccgctgttttaatagcacatgaagcataaaataggttcatagcagagcgcagcacagagcaacatggagcaactgttatgacctggagtgtctccagtccagcatgcaggtgataggtctcagcatttttgcacgcaggttaatgatggtgcagacggtcacttccttctctcaacagtcttcctggtcacgagcaccattgtggctcgtgtgtggggctcttttggcttagctctctgcacgagtttgctcctttagttcccagagctgacccttgaaatgagtgatattactcctgttttgtagacagaaaactgaagccttgacagtctgacgtgacctggcaagaggtgctgcagttggaaatgtgaattcacggctgacatctgggcactttactcctaacagtgttcagtgaacaagacgtcgctaacatgcgggggatggaacctagcaactcattctacaaacatggttcaaatatgttggtgcagggccttttgctttgttttcctaaagagattagattcagatgtggtggggtgctttgacagccaccgcaggacaaagttgatagctgtggggttgcggagtgttgaggatttcatagggaagccagtcctgcgcagtagtacgctcaggttcgtgctttcttgaggtgttccagaactggcctggagggaggctgcagtgtggaagcgggatttctgtcacctggagtattcttagaagttgcattctatgaagagtggagcatctgatgagctgtttactcgctgtttcatctgacggcagttgaaagacaaggcaggactggcagcgcagctgcctcagtcagcactgctgcactgggggcttgacctgcagtctcgcaatcctggactataactcattttgaagagagaaaaattaagcattaagtgattcaagcgtcttgcccaaggcgctactagaaaataaaggcactggtgcccagatgcaggtctgcatggtatgcaagcctgggcttcttcccacctcccccacagagagggcactggtatgttggagtgaagagccacgcaagacctctgtgaatgggcagagatgggccagtgacgcaacacagtaaagtgtattttggttataggcatcgtctctaaacttatgtaaaacattattaaaaaatggaaggacaacgatgaaatgatggccaaaaatatagaaaaggataccttgcatgtcctgtgaaatgcaaaggaattctaaagtgtcattatgagttacctcatggaagaaagcaaaaggtgaatctatctagagtttgtggttctgactcacaagagactgatgttcatgctgaaggacgagtgtgacaggtggaaggatagagcaccgagaccacactctaaagggtaggaatctatgggaactattcagggagatgaaagcatggaatgaactgaagcttgcagactcgttgagtaaaaagcgcgttttaggattggttttagaataaaataacaaggcctgtggttggggaagatgacttgctgttcacagagcctcccttaataggtggggacctcagcttttcctctgctgccatcaggtgagtggtgtacagtcctagccacagtagtaatcaccactggcctgactgagccctcaccctttatacagtgtctcctgccaccctcctgggagaggctgttctcggcacagctggcctggggtcacacagctggtaggtgtaaagcaggcattggagtccaggtagtctcactccgtagcctgtctctttagccactggaaatgtagagcaaagcgagaattgtccaaagagataagctaataaagaggaaaacaggctgggtgcaatggctcacgcctgtaatcccagcactttgggaggccaaggagggcggatcacaagttcaggagatcgagaccatcctggctaacacagtaaaaccccatctctactaaaaatacaaaaaattatccgggcgtgatggcacgcacctgtagtcccagctacttgggaggctgaggcaggagaatctcttgaatccaggaggcggaggttgcagtgagccgagatcacactactgcactccagcctgcgtgacagagcgagactccgtctcaaaaaaagaaaaaaaaaagaaaacaattatgctgagttccagagaagtgatgtctgtcttctcaggagagatgccgatgctgtctgagggcctgcccagtctccacatgattcagagactccagagatggacagctagtgccctgattttcccaagaggattctgagggtgacttctgtcaaccaaacaggaggacctggtgctgtcatcaccagttgtagagaggctgcggaccacctgctgtgtgtgccatcttacactgccatttgctgattgcttcaaagctagaggttgtttctaagagtgcttcgtgctaactaactaaaacataatgacattgtttttgtaaaactgatccgtggtttgttttttaaagcagaaagctctaaagtcactcagtcccaccacccagaagcacaagcaggtggctgccactgtaggacctctctctctaggctcgtgtagatggacacatggattggtcagtagaaatacttttattaaaagtcttatctttacataaatttgccaaattattaattttgcttgaaagggaaaggtgtccaacttcagttggaaatactagtttctaagagatactgctgagactaagagcataaaacatgatgaaaaccttaagtggctaagtgcagcaacgtcagagtaaaagctttgatgaagtcttggtttgcttgggctgtgtaggttggaggctcagcctttgtttctccctcctctggtgcccagtggtggtgtttgtgtccgtgaattttcaacctctgtagtttgtttgtagttatgaccactgttgcaccgaacccttaccgagggccaggccctgtgctaagcacttgccagtgtggatttattaagtcttcatgacagcaccatgtagggggagctgcgtccctgtgttgtacttgggacgcgatctgacctcaggcacttcaggctcctgaagggtctgcagtctcgtctctgcctgggaaaccacgatttgcagcatattccacagacctcatgctcatcatcaggaggcttcccgggactgctgcctgagatttctaagttcctaatgtggttcatgcttctggtgagtttctttgaggcgacgccccctgcgttgcctctggtcagctcagtcctgtggtcctgcagggcatcctacagtgtcctctgtctgtgacttgtgccgtgccaccttgacctggcatccactgtcctctaccgcgtttgcagataggagccactgttggtgcctttttgtcgtgtgtgttgaatggtcagggtcccaaatagctgattgggagcactctccttgaatctgccatgtgcctgggtctcaggtgactgggcccctccttgcttcagaacccacgtgcgttgcctgtcctctctggcttggagggttgtgtggaatcagaggtgagacccagtccccagggatgggcagttgcctttgattgcccagctgtcctcagcgccctccctcctgcgcccaactgctgtctcagttgcttgcttgaggatccggatatagactgaggtcggccttagcgtggcggggggttttctcttgagtcttggatactttgttaccggtcccatcttcctgtgggtgggagtgtctgtcttgcttggcccagcctcccagagaccttagctctcttagtacatgggctctgcctaccttgatccccagctcaccaccctagtgcagttgcttcttcgctcttgttccactccttgtcgccatccaccctgtgctttctcgatgtgtccttactcggtgtttctgtggagcagggcatcctgggcttcctttctgatccctggctcctgtgatcttccgtgctgggctccctcttcccttcccttttccactgtgttgccctcacacagctggcatgccatggatgtcgctcacccaagcccttcctaatgttgctcaccaaaacccctcccaccttgcccctgggaccttctccccttccaggctgcatgcaggccgagggcctggcgtctcagcaggaggcagtggggcctttgctggcacctgggctctgcatcctgaccttctgagggcttggtcctttaggtccatcttgaatctcctccaggcttcggactctctgctctgtagctggcccatggagacgggtacactcaggcctggtcttagactccgctgcttgggctgtgctggtgcctttggtgccctcttagtccatcccacctgggggccctgtcctgctgtccattgtgccagagtgctgtccccttgtctttccgtaactggctgctcgttacctttcccatctcagcctcagtaccagccacttggtatcagggaggctctccctgaccaacctaaagttcacagccgtggttgccagtttaaatttctgcatagcaactttttggtttattttggctacttatcttccccatcatgccccctgtccctcccatataaactcagtgagagtaggggccacatctcatcatcctgcccacagctctgctgtctgtatcagccagggtatgctgtgcagaggctcacggtaaatagctgcagaccacgaatcccatctgcttgctgtgctttaatattggcttacatctttggatccagtgagttcttttctctgtctccctctctctcactcgctcatacttactttgtgtaattggtgatttccagccttttgtatagtcctttctcgaatagttgttttctgtcatcttggcgggggcctcaaggggttgactgtacggagggcaggggctgcagagctgcagctgctgcctggggtctcacggcgcccgtgaggtgtaggcaggtgctttgcctctgagctgtctgtagaatggggtgacggcggtttcatcagactcagtgaagcatgtcatacagtgagtgtctggtcacagcaggaagatggtgaatgtcagctaatgagtattcatcaccaatgaatagtaacagttttttttactaaggctatgtaatgtagcctcagaattccactcagcacagccccctggcagcggtgcctctgagagctggcatgatggagagagcctggttggccttactggtgtggttggggcacttgggagaacgccttcctcacaaagctcatctggagggttttcggacttgtaggatagctttttcaggggccttgcctttggcagggcagggacgtgtactgctgcagtctagggtatgggataactttctaaaccagacccagaacttcatggccgcaggggccttttagccatgcggggctaggagctgacacagcgtcagcagcatgagggcctgtggtgctgggcggcagagcccagagggagcccctgctggtgtgactttagtgtaaaggctgggggataccagattcttacagaagacttaagacgggcacagtgatgtctgctctttgacccttgcagtatgaattagtaaaactgaaattattacatttcctttattaggattataaaagcaatgatgacttattgaagaaaatttggaaaatacagaaactacctataatttttccattgttaacatttgagcatatttcttgtcacttttaatggtgctttaaatatgtagcaaatgtatcatttcgtattttaaaaaaatgctaggtaagcatttcctcctgtccttaaaaagctcttttaaacaactttaaaatattgtatagatagatgtacacaattttctgaataattggagttatatttacatcttttcactctttaggaaaggactggcctgtttctgtgttgggttccttcctgagtgtggcttccagctcagtggctcagacttcaagatgaagacttcagtcctggttgtgtatggtcttgggccagttaccatatgtctaatgaatacttagttttgtcatctacaaaatgaaaatagtaatatttgcctcaaagactattatttgggaggatctagtgcaaatgttagtaatgtggatattgtgtagtgtcccaggatattaatgtttttagcctcttggcttttattctgtattgttgccccaaaagatgatgctcacttatctttcatccagtgtaaggatatctggaaagacaacagaaagtatagctgttttcatttcaaaagtgatcagctgcttgagctagcaagcaaggcttgcactagcttccaggcgcagtcacgcagtttcacagcaggcgcggttccctcggagcacccagagctgccctgcggtagtcagcagttgtgctgtggctgcactgccaggctgggtggcaggtggatcggagccagcagatgtggctcaggaagtgccttcttggcctctccttaatctctttcagagtctgtgggcccttgattgcactgtgggttgtttcagactccagtattaggagactgaaccccttggtggtttttttgtgtgtgtgtgctgagctgggttgaggacatgttaagcaggtggggtgcctcccctgggtttgctccgggtggtacctgtggtgtggggtggttctgagtagttctggccccactgctggagtatctgcccactcagtttgtgagatggcagggcttcatcctggtctggtgcctcattttcttctttagcagtgggcttagaaccaatgcagattcccaagttaagtattttttctgtagcttaattattacaggcttctggtacctaagccctttcttactttctgttctgaggggaagagaagataatgttgtttctccgcccccccccggagtggccccaggaccttgcatggcatttgcagcatttgcagcgtgcttgggtttgctttactagggtgaaagtgttgcaccccccagcacccacaaaggcacctctgctcacactccggtgaggttctgactggccctgggacatcacctgctccaggatcctatgtggctcatcccaggagagatgtgggagggaaggggaaaaaaggcttacatttgctgagtggaattcatgtagatctgagttccgcattgattcctaagctgcagagcccttatgccttggctgttttgtgaatgttagtcggtcttaacctttttcaccgagttagcattggctgtctcaggaggctcacagctcctgctcctcctccaggggagtgcgccctcctcctctgtcggtagctgtcaggtgcccctttcctctgcagcagactgtcctgggtccttgcctggccttccccttacacgtgagcctgcagcttcattcacagcccctgtgtagaaagataggcacatcgataggtccctccctgcccagagtgggcggaactgaggcaggcactaaaagcagctgactggcagccctagaaacatgaagggtttcatttatagtttcagtccttttccttctttcgagccttaatttaaaaaaaaaaaaaaaaaaagccttgaagtcctgcttctgagttttctaatttgtgcaggtattagttgccttgtaacataatcaaaaataaataaaaatgatttataattagcttattaactgtatcagtaaatggatactttaaagaggatcattgatccctcaaaatagaagcaatgcagtcattccctcattatgctttacttgtgatttgcttacaacccactcttcctagttaaagttaaatattaatccagaccctatcagtgcgatgtagtagtgtctgaatcagttgttgttttggtgtaatcgtatcaaagcatgttataaaatctacaaaattgcagggttaactccaaatattttcactaaggtattgtttttttgggcaaaaatgcatagtgaacattgtggagctgaagtgagggaacttcgatttctgagaaaccactagttttaagggttttgaaggaagagttggaggaggagaggaagagaataaattcacagttaatgagtttccagtattttctgtcgcattttacgttgtaatggaaaagactgggaactgaactcacatgcagtttgtcaaatcactttttccctagaattcaggattgatgagattaacggggtgttaaaggtaaactgaggcacataattaacatggacagaactgtagacctgagtgttgagagttgggaaatttcagtgagttgggaagactggaagcacctgttcttcagagtgcaggtcctcatattcagtgggtttaaggtgctgaaactttttttttttttttgagatggggtcttgcactgttgcccagactagagtgcagtggtgtaatcaccactcactgtagcttcgaatcctgggctgtcagcctatcctcccacatcagcctcctgactagctggactgcaggcctgggccaaaactcctggcttgaaacttcttgtaaccagattggaggaggagggcatgttcattttcgtgacgtttcctttcccttaaacatccagtgaaatctgacctttgaccatcactttgcttaaaagaagctactggatttaaagtctaggagaatgtcctagacaagcccatagtatgttcctgtatgttccccacccagagacctgcgttatgaagtgtttggtgtgcttcttccagccccactgttctttctaaagtgttttattttacatacgctgtcctggcttctgggctatgacctttgccttttttgccctttagttcctttgccctttacctactgcagtgcagctgcctgtccttgggttgactacaaagaagtcacctttgaacaacttagaaattgtgacttttggggaaggcagggcagagccttggggctgagatgggggaggagccagctctgccctgggagagatacaaagcgcctgcctgggtgaggcagtgcacgggtgggcttgcttcacctctttggccccagcttcaaaaccagccagtcctccctggctttggctttaattcacatttacaagcttgaaaaccagttaatcccattagctcagcttctaaagctgaaaatcgtcccctaaatgggtcacctgttgtcatcaatagctttattagctatggaataatatagttttgttctctaactgtaggatccttcttttgctcttaaaatagctcagtaagttgggtctcataaatacatacagcaagcatataccagatactaaaatacaaaaacattgctgatcttgcttttcagtactaaaagcagaaaatcgggaatttactaaattgagaagtcagtcattacctttcgatgggtttggactcttgcaaggcagtgattgtaaacgagagtgatcttttgttgtttttcaatgaactttattctctaatttttagtaaagcacactaggaaataatgcttcagaattctgttttcgagtagtttcctgactaaaataaaaattcactaaaaaaaacctctgctgtcaccatttccttttttcttaagataactaggaaatgaatcattaagagtttgctccgtggcaatggatcgggaagctgaccctgctctctgtggggctggagccttgctaatgtcccaggatttcacttcacagagacaagcatcagaggcttgcttcatttatagatcctacttcttttctacaccacagccaactcaaaatggtgacagaatctaagacgaggctagaagtcaccacggagcagttggaagctctgcttccggttctgggggcagtgttcctggcgttgtgtcctttggccccacctcagtttgtccgtttagctcctcagctgagaatgagatgtgtatcatataggagtttcctgggccactcacagccccaagctggagagtgccagcctaatgtgtcaggagtagggggtgaggccagagggctgtgtgccaactcctccctaagaagcctcctgggaaagccctcccaggcacttcccaggtctcactggccgcctgaggctgcaggggaggatggtcaggccgcctcttggctggcactgcttcccggcgtgccgccagcctttctcatggggaggggaatgatggcatgcctggggggcagcagggccccagggctgcctagggctctcactgtgtcctcctggttctgagatgccacctttgtgatccactgtagagggatttattctattatgatcaatgcatagaaatttcttcgatttggagactgaacactagtgagcagaactgaaattgagctttaaaagatattgatgacgggtctgtggataggagactttagtgtggtttttatgcagacgtcctgccagctgtaaattccctggaggtttggtatgtgagaacataagactaaacttatttctattttgttgaggagaaatgattaaaacttttcattgatgtacttctgtggcagactttttggagaattgaaccagcgggcatattcagtatttgaagtcatagatgagtaaaggaggtatgttgtagtttgcgctggcggcgtggcctgtggtcggcagggcttatctgtgaaggtatgtgcacagcttcctaaggcagtgaaaagtcctggcagtgttagtattgaatgagataatccaaaaaatgtaaaaatgtttacatttttaaagggatagttggcgatttaaatggtttctgctaacaaatcaaattattcattgcagaggtaaaatattttcagaatgttaattttagatgtcgtagagagtgtacatcagcaatgacaaggtcagcaaaatatcttagcaaaacttgattgattgattccatgcacaggcaagcgctgttctgggcaccggagacggagcagtgcgctgttgcgtccatccacagatggcctccagagtcatgcggttgcagggaggccgaagggccagggaggccgctgggtgggcacggctgggcggtgccctcacgtgggttttgttgggctactcttactgtgcactttttctcagtttggtcagtgttccgccttgctgcctggcccccaaccctcgccctctgagggcctcacaaagagccaagcagaaggcaggctgggggcttttcaggccagcccagaggatgtaatgatgatggttggcactgtcccacggccgccaccagtctcacactgcctcgtgggcagtcctggagtcgtgcggcaccttgctggtccctgctctccttgaagaagggccagtggggcacttcgccagagccttcttgtctgactccgtcatccaagaggcatggatggccgggcccccggcagtttccattctattctgagaaggcaaaacaaaattattcctgtctcttattatctaatatttgttacagcagttgctcacttttaggtgcattttattacagatttcagacaggtgtggttattagtgcagcttactgtttggacacaatgccaaggtcaggaggacagtgttcccctgagcaccacttctgctaggagcatgggcaggccatgcctcgccattaatctctcctgatttaggggaggaataccaggccaccccctcttctccctgtgcaagggaacagacatttgacaaaaacggatgccatgttacgctgattttgtgtgtctaaggcagactgcagcaggtgttatctccgtgctcttcctttcctggagtgttgagcatctcttgatagtaggggatgcccctgagggtggtgaatgtggctgcacaggtcctgaaagctatttgatgttgccgttacttcaggtagaacttaaagttgacagtacattctactctgcaggtggaaatgtggagtgccattttgacaaattggaatgccctgtttacaatatgctttaatgagttaaatctgggggatgtggatagaattttagtatcctagctttggcattcttccatgactttgggccaattatttaataattccaagcctgcatctttgttagaatctctaaatttctctactcctgttattatcctcagaacaggactgtgaggtgcagtaggccacatggtgtagtggtttaggtggacagacttgccagtgctgttctcatggatagcctaggactgtccctagctctctgcagtgacagtgatagtgactggtgaaggtgaagggatccacccagacgttcttcctgatggagagaggctggcctgtggctcttccctggggtggatgttaacctgctaacgtgacatatctagtcctgcttacattactaagtggtaggaaattttaggtaacacctcagactttaaagtggcctactgagctggtagaaaagtgtgtagttggtgctcagtaattgttgaaaagatagaagctttacttcaaagctcttgtagtttgatcagtttggaaaaaatattttaatgttgggctctgttaacagctggactggtggctgtctgaattgggaccatgcttggggtgaggttttttcttacttttttttttttttttggtgagacagagtcttgctctgtcgcccaggctggagtacagtggtgcaatctcggcccactgcagcctctgcctcctgggttcaagcgattctcctgcctcagttttctgagtagctgggactacaagcatgtgccaccacgcctggctaatttttttctatttttagcagagatggggtttcaccattttagtcaggatggtcttgatctcctgaacttgtgatccatccacctcagcctcccaaagtgctgggattacaggcgtgagccaccgcatctggccacatatttttttaaattaaatgtgatacataaaattaggctgcaggcatggcctgatttgcaggtgcttcatgagcaagcgtgcacagcatttatttgctgctcctggagtctctcgtgtgtgcttgtagcctagccttaagccctctgtggacggcttggaatgcgtactccaaatgactcttttggcggggtgggaagtggcaatactttaggtgactgacagttgaaattaaccttacacaagagccaaactgtaggctgatgcagggccactcacctttgtactcacccctggcaggtcctgtaagaggtgctacttgcttccactttgccagctgttcgttggccctgttttgtcttctgctgtttgccttatttatgaaacagaatggaaacaggcgagtttgatttgttataattcctaggagtcatagaatggaagcaggtgagtttgatttgttataattcctaggattggataatttgccttcccctctctccatctttaattaatcccttaaaggaaaagaggacgacagcacttttcctgcagtcatctgtgtaggcctcagccttaactcatgacataggctggtgccactggccacagggctgacctcagctcttggagcccatggggtgacaggagatcagcacctttgaggtggcggcgtgaggcgtcttccaggccttgctcatggtgcttgaaaacactgctttagagctttgttaagagagagaggccctacttactcctgtcccacaggcatttgggtgttgacctccttgttggcctcttaaggagagaatacttgagtattgaattcgatcagcttttctgcctccagggaccctgtttctctctgctgagactgtggcggatgaaaccaggatttagtggatggtcaccaggtagcttgggcagacctgggccgcggggcccctgaggactcatacgtctttttcctgttcacatgttccttccccacaccttggcccattctcagtccctcccatgctccttagcgtgaggcactcagggaggtgcccatggggccttggggcctgtgctggagctggttcccagcctcagaagtgtgcctaactgtgccacattcattggaaagctgctttcacacttcctggatggaattctctcattttttcaatataaagtcactgagatgatttttttggagaagtctttaaaggccagttatacatttattgatacttggtattgacaaagttcagttgtttagtgttgctaagtcatactgtgtaagtttgttgagcacagagttttcattttatactttcagaggagaaatgaaactgaatttcgtggccagaaatatgtttgagcgtcatcctgatgtaagaacagaacagaaaatgtggtgacatttcattgtaactctagtatgttttcttttttgtccattagactacatgaggaaataattgacttttataacttcatgtccccttgtcctgaagaagcagctatgagaagagaggtggtgaaacggatcgaaactgtggtgaaagacctttggccgacggctgatgtgagtatgttctttggagttctgtgtcgcacgtcacgtgcgagtaaatttaaataccctgtgatgatgatgtgtcggctagatccacacagaccttttctcgttggcccgaggcagcagttctccaagtgtgctttgagaaggcctccgttgcctggaatgcatggcccccggcgcacctgcacctgctgtcttagacacctgcggtggccgcacatcttcgtgatgctggcgcgcactcaagtgcgaagaccatcggctgagggatttgttgggttttttttttttttttttggaagggggagaatggtgtgattgtttctcttaagttcaccttaaaatttagaaatttcaccctgtcacccacccctgcttccccaccaccacacatagtagcatataatgtgctcatttttgtaaaacttggaagtgttccctcatcacacaccactcttgcagtgaaggaacaagtgttttttgacatgtggagtggggccttctggaatgcttggtgcaggtggcgtgaagcctgcccctggccggctctattcagcagccttccctactgctgactgggctcagtggaccagcagggctggccgtgccccagctgtgaggggcatgtgtgctcttggtgggcaagggcaacccagttttctgcgcctcttttaaaatgatacagatttttggctttaaacttcagagtcctaggacaaagccctgccccagtgccttagctgtgggtttaagaagaggttgaagggtttgaagctagctctgaaaagtcctcagctttgaagggttatagggtgaggacaaaacttgtttcacctcttaattttgagtttttaaacattccttttttgggcatgtctttaacttaaagggaattttctggttgattgttatacggtcccctccatcagtttccaaggtagttttattttttacccaagggtatagtgagggctttcttttagtaagaaataatggtagtgtgactgcttggtttgtggtatacattttaaggcaaccactctttctttcaggtacagatatttggcagctttagtacaggtctttatcttccaactaggtgagtaccagactgcatggcatgggctagtggggggctgggatggtgtctatgcaatattaagggctacaaatagattctttgtaattgagtctaaggcgagaaatgccagctaaaggaaaagactgtggttacagagggaaattggcagaaagatttaatttagtgttatgatgaattcattgctttgcatttttcctctcacacttaatttgttggggtgcaaaaatgcttcatgctggaaatatgaagaagacaggtgggaggactgtgggaagtaaacgcaatagaagacattctgctgatattttaggacatgtgtttgaaaaattgatcttatgttttgatgaagattcaagcaaaattctctttaaatagtattttctaagtatttttacctgataggaaaatgtcaaacaagtttgcattctaaaatacaaactagtattttctcattaaggattcttgatagccaaataaatttcctgtgcactgtctcattaaaagcttaccttctattaacccactgctagttagcatttggaggccgaagaggctataatctcaggatttgggggttgacattagcagggccagtgggtaattgaacaggtttgggtatccaggaatctctgggtccgcaggagtgatccagcgtaggcagtgccggagtaggtgctgggagagcggggcctcagcctggtttgggggcaggcattttcatttgaatcccctcacatacctagtgctgttgggagaatgatttaaccttcttgctttccatcttatgctacaaatatggaagtcttcccttaagttcagcgaggacttgctgtagttcatgaactgcacttcacctccttaggggccataggctagtgggattgtgtcttggcctttgtgggagacacaggcacatgtgccttggtgcttatcctccccacacgagtgtgtaggctgtggcgaggggagcagtggctgacgtgcgttttcttctgtcagcgacatagacctggtggtcttcgggaaatgggagcgtcctcctttacagctgctggagcaagccctgcggaagcacaacgtggctgagccgtgttccatcaaagtccttgacaaggctacggtgagtgcctggctttggcccctctgaccgggcaggagccttgtcacatcccaggtggtcacaggatacgcctgcgtcacgagcttgtggtattttacacagttattggctacagttttgaagattaatctgcttctggtatagacatgtgttttatgtttttgtttcatagtcgtgatcaccaactgagaacatgtttagtgacagtctgaacttttgggacttgtgagcccattaaactgttcttggaatgaaaatatatgattgtgtctacttgtgttaggatgaataggaaaggagagtcatctgaaaccggacactgacattcaggtgctgcccattatggagagtgtggctcaatgattaaaccatggtttttatgattaccatttgctatgttatgttaaagaggaacaacttagctgttcctttcgtggttccaaaaaatatacatatatgaaaagccttttatctttgggggaagtttgaagatgagactgtttctggtgtgtacttgctaaggtttatgtcagttcaagattataagccccccagggactatgaggtacttgcctgttgtatgacagttcttagctcacctgtgcgaccggctagcatttcatttttaaatttgtgtgtcaacttgtgtgtgattcacatccttatagtgtttagcagaatgtaagttaaggcaggagcttcctcctgcctgtgtggtgataggggagggggcattcacatgtttctcattgtcaggtgcttttgattgaggctgggggcaagttttaaaacatgatatatgcactgaaaagtgcacatgtaccaggtgcacctcttggtgagagttaacgaagtgcacatgctcatgtcactgtcatgtggaccagagcgagcaggcagcaccagaggttccctccaggcccaggttccagaaggggctgttggctcttctcattagcacgagacaagccctggccggccactttctcaaatgcttacgggccttattgcacttagctctcccagccactctcatcagcagatgctgttgctgttcacattttacaggtgaggaaactgagatgcggagaggtgaggtcattagcccaaggtgggtcagcggcatagtccaggccgctgttgtctgctgctcctctgtgtgtgccaaggggcagcggggaggtgggtgggaatcctgaccaggcgaccacctttggagtagaggaactaaggcgcggctgtcctgaggccacacagtaggttgagcagttacagtaactgcttagtcccagtgacctcgttactgtcgcatattggctactaagtatcttttcctctgttacctgagggccagtgtagactgtagggagagcctggagcctgccactgctcatttctggggagcactgtgcagccggccagtcatgggcacaagagacccagggcgagggctgagtttaaggtgaaatcttgtctatttggaacaacaacccaaaactgtgttacagtgttaactcctcacctccaggatttggggttcctgccgtgagtgagtgtgtgcaagagtagggcaggagagtgccaggagtgattgtgggaaggagtctatgagatggaaaggaagctgcttcctaaactgtgggtgtcagggaggcatagccattgagtgttggcttcttccaaagcagtttctgatgagttctttgggaaagtatttctttctctgccttcttaagaacatactggcctcagggcttaccctgcctggtggtcgcagtggtgtccaggtgactctggccccactggcatgtcctcacacgctgagtccttggttggttgccctcaagtgtagacattaaagcccagaataggtgtgtactgaatgcactgtccctacgttgcctatcactggcatttgaacttttcgttagacactgattgttttggttaaaggaattcctttttttaatccttcaaggctgaaggaaacaaaacattgccttttcttctggaagaattcagttttactggtggggtggagggtggggcatgagtgtggtctggacagttgctcaggcagattttgaatgccattcggtgacagttcttgttggtgagaatatttctaaaatagccttttatttgctgaattttttaggggaaaaatttttttagtaaagttgtcttaaagagtgaaaacccaaagtagagaaacaatatcagtataacatacaatttaaaaaatggcttcgaagttacattaaatgatttcaaaaattctgccaaataaaagtctggggccaagcacctctctccatcccagcacatagggtgggcgtggcagagactaactcctgttccctgttggctccctcccctcacgtctctttgatgcttggtcacctctggtgctgatccagggctacaggggctgggcagaatgtgggtcctgctgtgaggggctggggcctgggagtcgtcctgggttcagggactactgatgctgacagtgttctctgaccccctttcattactaagaaaaaacaccaaacctctgtacagctttggcagcattttgatgcctggctgtggagagtcctgcatgttaaagcagtttttaaaatgaaatctttacaggtaccaataataaagctcacagatcaggagactgaagtgaaagttgacatcagctttaacatggagacgggcgtccgggcagcggagttcatcaagaattacatgaaggtactgtgcttggtgacccagcgcggcgagagtgcaggactggagtgcttgtgcttggtggcatcctacgatgtttacagctgtcagctgcacacacaagtctttcgtaacacagactacacttacattatttctcctacaatattatttctagagatactttgaaattacatagctgtttttaaaatttgcttttcctgagtaaccattttataaagttgacaattattttagagagctttgttaaaatgttctttctagttattacagactttgcttctagctcagtgtgcctcattcgcaggttttacccaggtggaagttgataaatcatgaggtgccttaaatatactcactttgggaggtctcagtgcctgaggatgggcagagagacttggttgagctgacatgtttgggactctgaccatgtgcctggtcttaaacgggtggtcatgacctcctgttacagtagaggccgtgcagtccttagcaggggcagacgcacctccgggtggtcgtgacctcctgttgcagtagaggctgttgcaatccttagcaggggcaaatccacccctgcaatctggtcccatcttgttccattttcaagggccttgctcttcaggttcccccttcccccttgtcatctggtctgagggagcatgtccaccccaagcgcaacacgaacagcagcagccaggctttccctccctcctgccatctcctgccgctctgccttccttgccagcctcactctgctctcctgctccggaggcccccactgtcctcatggcctgtgtagcaagcacataaacactccagtgaacgcgctggtctcttcctgagttcctttgtgcctgtggactcgtcatggtaggggtgcacccctgctgagtcgtaacccagggagagctccacagtctcgattttcacaagcccctcaggaaattaattcttagcgtcccccaaccaaagtttgagacttaatgactgagagccttcagatgggcaagaggatcgaggaaactttcctttctgtcttgtgtgatttgctatgtgaaatctctttgaaagtatggtaattactcagtaaatctttttcttttggaatttacagaaatattcattgctgccttacttgattttagtattgaaacagttccttctgcagagggacctgaatgaagtttttacaggtggaattagctcatacagcctaattttaatggccattagctttctacaggtatgtatgctttcttgagactgtttctgttgagacatgtgtaagagtagactcttccaaccagttgcctagtgggttccagcagcctttgctctccttttactgtattgtttcaatttggtagaggctgatttctgattcttacaatcaaaccctcttgattaatgcacctttctggatgctcattttgtactgggtgtaactgttggtgcaggggtgcccgtctggttctgtgagtccagtgcacatcagtccaagctcagggaattctctgtattcagaaatgtccatttcatggtaaacaataaacatttcttggtgcttgtctgtgatttatattgaaaaaaatttgtctcagaataaagttgagtaccacatatgagaaaaggatttacaagagagctttctcagactgatgaaacatcattattttgtcttaaattatatgtggtccttattttgctgagtaacatggaaaatctatcaatagaaaacctacgtgttttaaaaagtattgttaaatgctgtgatgtattgataaactgtaattatactttttaaacatataaaatcatctctaattggaacagtaattattccgtttatattttcttgagtgaagaattctgtcctttacaaaatcttgcctatataatttagccgcacggctgtatttctccagtgtttaccattaatttggtctttgtgattgtgctgagattaccgaatctgtccatgatttggtaatgttctcactgtcatgaatgctatgatagtagaatcactgggtaactacctgtgatgatcgcagcttccttggtctgtctctgccaagatgctttaaaagtagtgaaaatggaattccatgtctgttttcttacagttctagtcacactgtcctgtcttcactttcccctctgagatgtggcccttatatatagcctttcttccatagtttttggacattattttgaattaaacatgggcctctgttctttactgatatactcctgacttgcactattttcatggccttgtagtaacaacagctacttaatactttgatcatgcatgtgccatgtgccaggcctgggtccagggcctcctccaccccctcagggggtcctttcagtgatcctgtgaccccacaggaggccaccacagcatgagtgggattcctgtgttcactgccacatccctgtgccttgtccagtgcccagagcttgtcctgcctcagcggtgtgactgtggcagcatctgagtttcttgactaagtgaggggaggaggcccaccctggcaccggccaggctcttggatatgtgattttggctaaaagacaaggaataaggaagggaataaaggtcaggccagaatcggaatcctcctcttgtgtggtggaaatatgcagagaactccggaattcttctgaaccctaaaaacatttgtatgctttctagctgcagctcctcctggcactctgtgttataaatagtttcaagcaccgtgcttctctgagggctttctctcatgtgccctcgtccatccctttcgagttggcactgtccgatagaattctgtgatggtgatggccacttctgcatggtccggcaaggggcagggcgccacatgtggctactgagtattgcagtgtggctggtgagagcaacaagctggatttttaattaatttgttttaattcatttaaatagacatgtgggcaatgcaggtctggacagctgagaattatgaccctcaggaggtgtggtggacagtggtttacttccgaacaagcccagtgcctgcttttgaagacgatatggcactgaactgagagtggtgctcatctgtgtgacaagcaggatggaagcttgctataaatattgtgatataatgctaatgacctttacacagctaaaatcaatcctttcacttttccggttttatgtgatactgccatactagtcagtctaacactgacccctgttggttgtgcttcagcataacgaaattaggatgacgagaatctgaaattacatctaccatccaggtgactaagttatgcagaatatagtccaacttatttgcccatatttggttaatcagatatctgtgtttgcaggaagtttgctgtatggattaccaatttcaaaaatcaaactacactaaaattcagatgagtccgttgtgttccttttgaacactcctcgttgaagaggctgctgttcaggcttcctcgtggtgctggtgagtgagcgagtgccactcactggtattgccttgaagaggctgctggacagcagacttcgtcgcgtgctattttctttagaacatgccatgaatccatacaaattgtgggtgcattgcttttacagttgcatccaagaattgatgcccggagagctgatgaaaaccttggaatgcttcttgtagaattttttgaactctatgggagaaattttaattacttgaaaaccggtattagaatcaaagaaggaggtgcctatatcgccaaagaggagatcatgaaagccatgaccagcgggtacagaccgtcgatgctgtgcattgaggaccccctgctgccaggtaagggcgccctgatctccactgctgagagctgggccagcctcggggacgtgctggtgacagggcctgtgttggggctctgagagccccgggcagttcatttgctcttgatgcaggtttctcttatactaaccagttaataatcacactttgagaaatccttatttaatgcttctaattaacttttgtctttctaactgtttacattctataataaagaagaattaaggaaaatcttttctttttctgattattgaagtataacatatccaccctaaaccatttgtttctctgaagtatgtaatatagaaaaaccatggatgccataatccccttttatttggacatgtatttctccactttttccctatatggatattaatgtttattattgttatcttgttgaacacatcccacaacgcaaacacgtttgtcacttggcttttgacttaacgcttacccttgaccctttcctatgtgtgtaggttgtgttttgtcgtgaggattgccgcattgaagagtctaaggctgggctagaggtttcccggttttattcccattggcgtattcaggggtatttatgtcctcattcctttggtgacagtcttttaaaatctttgccaatctgataaaaccatgtgttaacgcttttaaatttatatttctttaatgattagtggggttaaacacattgctatttatgaattccacaaattttgattgagcgtctgttaggtgagataccattcttactgttggtgataaaatcgttggcggacgggtgacagaacaggtggtggagagcccagctccaggcgcgtgtccctgggccttctcctctgtgtggctctgtgtgctgtccgcccggttgctggctgccctcctctgctgtgtgcaaggcctgtgtgtgtcggggaagcctttcttgtcacacatgtcatactttttccaatttgttttttgcatttgtatttttttattcagctcctatacatttaaaaaaacacctcagatttaatggttgtttttgttttatggcttctgggatgtgagctttatttatttcctaaagatcgtctcaaggaaaacacattggctgatgtgttttgttgttgtttatagatggcatgatttgtgcaggctcttgagtggttttcttggtagcacggtatggcagtgtatcatccatttttgtccgttggatgagtcacattggagacattggcattgtcttcaagtgtctgctgaaatgtactcaaaaaacaaagacccacataatcgttgagttataatataaatctagaaaagataaaactccagtacttagaacatatatcctttaaaagaaatccacacttaacctgattgtgaagaatgagttgtttgtagattaaaattagaaacagtgcttttcttatgaaaattaagcttctcctgactggcttccttggtgactgctgtgacagattcctttgatttgctgtcccagttttccacacgtgagaattcacattccattctaaaggatttacttcctgtaggttccattagcgttgactgagttgtgatgcacttggggtgagctgcccttctacctgccctgttggggcactgtgaggcctcctgattgtcagatcagcgatcacagtgggtcggtgctgctggtctgcacaatggtagtctttggcttcctcatgtttctgccacctggagaggtggctttttgtgggctgtcactccgtctgtgaatggcagccgcctccgtgaggtgggccatgagcacaggggcactgatgatgcagaccagtcctctcggcactcacagactgtgccctgtgcatggtgattgacagggcctttgccagtgccaagtgccacccgtgcccattgtgttcgcctgtctgggctttgctggtagggtctggaagagtttcagtggtgagggcctgcttcagagtcacttgtatgagaagatccaaaacatgtggacgatggtgtgcatgtggggagggtctgacatagcctttttgctgcagggaatgacgttggccggagctcctatggcgccatgcaggtgaagcaggtcttcgattatgcctacatagtgctcagccatgctgtgtcaccgctggccaggtcctatccaaacagagacgccgaaaggtaatgggttgtgtgtctgcgtctgggctcagcgtgcctgtgggatggtacttatccctttcctgtgtcatttacctccatgaaatttatgaagggatgttctgccgtatttcagtagaatctagatatgttggtgaaggaaggccttctaggaatatgggatggctgtgtgggattcatccatggttgagagttgaaaatttctttcttggagatttgacattttcttcagggtcttttgttttggggaggtgatttctggcttttaaaattcagtccctaccatcttctcttatgtacactcgtcccttgttctacattttggggcatttttacagtcccaaaatgtagtcagaagtatttacttctcacccagatcattctgtggtagtggaaagggtggtatttgaaggggtgggagatgagataggaatgggaaggaagagtaacgtggtcgtcaagagtggaattcgaaacagtttgatagatctgttctgtggtggatgatggaataaacaggtttcgaggcctggctcagcagccgctgcaggtgctggtggtgctggagctctgtgtgttcctgagccgctgtctgctcggtgttttcaggcggagctctgggccccatgtagggcactcgtctcggtaccgtctccattctcgtccgtgcagtgggaagtgaaatgtcagcactgtatgatcatcgtgggtgggaaggccccgctcccctacttggagctgcatttcacagtggtcttctgtaggtagatgtactgcgatcccagggtatgcttgagctgaatcattaaaagtcagagatatttgtcaacgtattttagctcctttcctactgtccttcacctagcgagatgatctgttaggggtataaggtagctgttcgagaggggttctcagctccctgacacctgttgtactctgttgatctccaacaatgtccctttgcagtactttaggaagaatcatcaaagtaactcaggaggtgattgactaccggaggtggatcaaagagaagtggggcagcaaagcccacccgtcgccaggcatgggtgagagattaattcatttgtgttcatcctaaccactggctggcatgttcatgcagaagtgtctctattcctttgtggtaaattggtcaaattaagaaaatagctagtttttctgatgagcattaattaagaagacaataagatctagagcagcactgtccagtagaagcaatataatgcatgccacacatagaatttcaaagtttctaggctgtgtcaaatgtgaaaagaaacaggtgaaataattttgatagattttattccactcaagtcaaaatattaacatttcaacatgtaatcaacataaaaataattaagatattttatagctgtttcttgtactgtctgaaatccggtgtgtatttattcgtacttatagtacatcctaattaggatgctaaattttcgtaaaaaatacttgatctatatttagattttagaaagttcacagttgaagatgatttgcatacccaagttattacaaacatgtttaatgttttccaacaactaattgaatgtaatttttaaaattaaattaggcaaaacctaatgttgggtttgttagtcacattagcagcgttcccggctcagcagagcccgtgactgatgctgccggggcggctccacgccgcagttctcaggagttattaaccaaggctttttccctccacagacagcaggatcaagatcaaagagcgaatagccacatgcaatggggagcagacgcagaaccgagagcccgagtctccctatggccagcgcttgactttgtcgctgtccagcccccagctcctgtcttcaggctcctcggcctcttctgtgtcttcactttctgggagtgacgttgtaagtgccctcccctcctccgtgtgtctgttggacagtttgtgtctctggtaaatgtccatagccgcgagcttaaaatctcccccttggttttgctcaggttttgtttccttgtatgtgtgtggaggtgggtggggggcagccccgtgatgtgggcaccaggcttcctttcccctgccgtgaaccttcagaacctgtctgtgcgactcatgcggctgtcgagggcagtaatcctctaaatggttgaactacagtggacttccttgagtagtttttaaaaatttatttgaagattaaaaaaaaaattaaatccaagtatctcttctgtatttcctttaacattctttttcagttgtgatgaaattacttgaaggaagcctgggtaggtttgggctgcctgttcagaagttagacttaatttgaataacctttcatagccagcctggatgcaggcgtttcttttcatagctttaaggaagtagtagtgcacctttgtggtacagctgtcctttttgttttttgtaccgggttcaaggattcagacacaccgccctgcacaacgcccagtgtttaccagttcagtctgcaagcgccagctcctctcatggccggcttacccaccgccttgccaatgcccagtggcaaacctcagcccaccacttccagaacactgatcatgacaaccaacaatcaggtacgtggccctctggcacccttcccgctggtggcccctgggaacagcatccgagctgtgatatgcactagaggagattgatggtcctttgaattagaagagtaactttttgagtatttggccattggtgtgttgttctaggaaatcctctcttttttgtggtgttgaggtcccccatgtatagtttcagcagcgaggacactgtggttcttgagtgctgccgtggcttttcacgggggccaggttgactgccttcctgcaagtttcctcactgccccagcatgagactgctgtcgagggtcatcttgagagagcgactcagtcacgacccacttagctgggcgccaagccgtgccagacacttgtccctacttcctctcagaatctcaatgaaagttttaatgtgaacttattagacttttttcatgtttgaaattaggcataatttctaaggctttttctgttggaatatactgtttttaaaatttagataaaattagaaatctaaaggataattttataaatactaaattttgtatctacttgcgattatacatcacttgaatatgtgtgggtataaaacccaacatgttaattgacttaaaaccattttctgaaatgtggggtataatttgagcataaagctatgtaggtacatgcaaaagtgttttgactcatttcttggagttttgcactctgctctggggaagacattctcacaggatccaccgtgattctggcggagcttctgggatgctggctctgtaatgacccacagagctgatgagcagagccatggcccagccggacaccgtaacgtgtctaattgcagcataagtgtaaaattcaggggcaattatttacactcttaaaatgaattataccacagataaacttggtcgcctttttatggtcatcacagtggccctgacgtcctggccatgtgtcacaaaggtgtttgttttaaccacccacaagccttggggcccttgagagcccagtgcggctgctgagctacagagccacactctgcggctgcttgtgtggttcgagtgtgaagtccagggacgctgagggtttataggtttttatctaagaagactcttggccacagtcaatctccagaggttgttggggtaaatgcacgggatgccaagatgcaaccaggtcagtattgcaagtctgagaaaaggggttctcgttagcgcacttctgctgctgacagtaacgggtgatgctgacatagaagcagcctgggacctggacagcaggcaaggaaggaactgccagccgtcccacggcctctcaggccaccagttgggccagccttgggctgtgacccctgagttcagcgtgtgagtagggggttcaccacgggggtgacggttgttcttctgatgactctaatgtcttgatcgtttgatcttcaatgagtttcaaactttatgacttggattactgggcatactttatatgccagttgctgttttagaatacgaagtatttccaattcaaagcacaatattgttaggtaatagtaaaacagactgctctatggagcccacatgcaactgtgccatttatcagctgccctttggtggtgctgagcttagaagccggatggttttcctctgattgctttggtacccatggccgtctctcattttgttcctagaccaggtttactatacctccaccgaccctaggggttgctcctgttccttgcagacaagctggtgtagaaggaactgcgtctttgaaagccgtccaccacatgtcttccccggccattccctcagcgtcccccaacccgctctcgagccctcatctgtatcataaggtatagctctgtcctggtgcattcacctacctgttcaagctgccatgtgagaggcggtgctaaatgttttctcctccagagagaattccagagagatcatttgaaaacggaatttgctttgttgtcattcagcctgtttgcttgtctttccaaacaaaacttaaaaaagttaaattattttaagatgtaatatatagtttaattggttgccacaaacatctcttaattcctctgttgaactgattagcataaaactgaagtttgaaataaggctcaaaatgaagacttttcccatttacataattcatttatatgctaaataccttggttttcaagaagcaaatgataaaaccaagagcagatcttgccatgatgtcccgtgtatgctgctgtcattcccacgttgcctgatccccgcctggggcaggagcaagcgtcagggctggcagagctgtgtgctgggcctcagcagggccctggcatgcgtgcccttgtggctcctctcaagtccagctgtgtgcatggaggaaacaggtcacgttaagtctctatattcttgaagtacctgaatgattgggagagccatggcgaggatcttccaggtcagcccccgtcgtgtgtgatgttccttgggctctgcggatgctcggtgctttcatcggtgtccacacctctttattccgctcctcctttgcttgtctaatcctattttgccagtaagttttttattcttgaggctttgttggccctgtgttgtatgatgattgtttttaggagttaagtaatagaacatttcctcttggatttatccatccccgatagacacattcagggtgaaagaacaacttcgcacaccggcctcttctttgcattttggctttgctttcccagtctcctcctgctgtttttcttgctctgagactttcctgaagccggcgtgtgttccctctcagtctgcttggccgcgactttgcagtgcagggaatgtgctttgggtgtagcccaagcacaggctgctgcatgctgggatcgacaggctgctgagggcgagagcgccaggtcctggcacgtgtgacttgcttggttctttctagaaggtcacagctgggggaagaacatgacagggaccttcttacttctgtttttttggagacagaatctcactccatcacccaggctggagtgcagtggtgtgatctcagctcactgcaacctccgcctcccgggttcaagcaattcttgtgcctcagcttcttgagtagctggaattacaggtgtgtgccaccacacccagccaatttttgtatttttagtagagacggggtttcaccatgttggtcaggctggtctcaagctcctgacctcaagtgacatgcctgcctcggcctcccaaagtggtggaattacaggtgcaagccattggcacctggctagggaccttcttatttctatggataagtggaacaagttagaagtgaggttctgctgaatttgtgtggtttgatcctggtacatggttcttgcctttagtcattcacggaatgggaagaatgcttttctctcagatggaggagttgggaagtcccagagggcaggtgtccatccctgctctctatgtaacatcacgtcggtgcttagtgtggtcactgcccgaggacgtgggcattgtgcctgctgtctggctccaacactgctgtctctctctttctccagcagcacaacggcatgaaactgtccatgaagggctctcacggccacacccaaggcggcggctacagctctgtgggtagcggaggtgtgcggccccctgtgggcaacaggggacaccaccagtataaccgcaccggctggaggaggaaaaaacacacacacacacgggacagtctgcccgtgagcctcagcagataatggctcctggctgcgtcagcctcccccacccctctgcagactgccccgcggcctcggccaccggcaggggaaccgagaccagcaccccgcacgtcagccgggctcgcggcacgcccgccgctgatcactctgcatgtttcttcgtgtggtggtcgcgtccatcttcaagaacagctcgttgtgctcatctgtgaagccttattaaacgtggacgttgttttctgccttcccaggattcttccttcagtgctgaggcaggtcgggctcaggaactgcagggacgtgaacatgcgcttgcggtttgaggtagccgtgtctgttccttcgcggtttgctattttcatttcctgttcgtcaaagcagcagaggagatcaaaccccgttcgtgtgtctttcctccacggataagcttgggaggtcattgttttactgccctcacattttgtttgaaatttcagaactgtttttctatgtaaatattgaaaacttatgatttgtgcaataactcagatattttttatttaatttcctattttcacataagttatatttaagggaggagggaattttttttaaacaagcttaggtcctttcccgagctgcattttctaagttgggtcatcgtgtcggctggttgtctgacgagcatcgttacaaacaccatgatgaggggtttggggttttattttgatgtcttttcttttggtcggaagtgagtgaaggagccaggtcgccctgaaggttttccaaagggcttggctccagagccacctggcagactgcccgtggccctgctgtcgggccccaggccgttgtcctgctctgaccacagagttttaatgttttggttttcacttcttttaaactggacaacaaatccagcatttcaagtgccagaagtataactttctaaggagagaagggttgtcacattataaaatctttaggaaaatgtgaactggaaaacgcttcggtcagttttagtgacatagcctgtgatgatgggtctggtgactattattgcggaccgtggtacccagttttaggaatgtggagaaaggaattctgttgattccgttgaggaatctgtagcgtatgcattcgttctgttaagagcaaatctaggagaagtgcttcagctgcccagtgcgccgtggggagtgttttaacggatcgtgtcgcaggagagcacagcccagcgttggggccgggaccgctggcgcccgacgtcggaagcatacaggtatactatgcaagtgtattctgccacaacaaccactgtctttgttacctttttttgaacaagaatatatccatcctgcctaaccctgagtttttggagcaccacagttgtcctgggagttggttgcatcttgtaggccatctgacttcctgtttttaaaacgggggtctggtcttgctaaacactacaggtaggttggtctttgaagtccactagtggagaatgtcaagacaagatacttattaccatgacatctgatgcatgtgcagcagtggggagttctagattgatctctgaatgtgatcgacgcccagcaaggacaagctttaaaatgtctgcggtctgcccttttgaagcaggactggctcactctgtcattgggagctgtcagctgcgactgcaggttctctaggaggcattccagaatagagtagcacactgtgtctgcagttctcgatgaccgaaagttatcaaaaatatttaaaatatttaaattgtgaacctattgataaagaatatttataaaaactgatctgtaggcctgtactaatctctacgcattagcaatattgactgtaaacccacattaaggaaaccactacgggtctggcagtgcgtgtcccgtggggtgtgcattttaaaactcgattcatagacacaggtaccatgttccatttccgtcatggtgaagcaaatgaattggcctggctaccactgtggtcgcgtgctacaggtttgacaaaaagatatcatgtttcgatttttttgtgtgtggacaacaatatggaagctaaaattgacatatttttatgtaaagtttttctattctttgatttttaataaactttggaaaccagttt

SEQ ID NO: 3 - DNA, 86750 bp, Macaca fascicularis
  misc_feature 23430..24809: note = n is a, c, g, or t
  misc_feature 70678..70758: note = n is a, c, g, or t
  misc_feature 71318..71417: note = n is a, c, g, or t
  source 1..86750: mol_type = unassigned DNA
ggcggcgcggcctcggccccggccccggccccggccccggccggcatgtaccgctccggggagcgcctgctgggcagccacgcgctgcccgcggagcagcgggacttcctgcccctagagacgaccaacaacaacaacaaccaccaccagcccggggcctgggcccgccgggcgggctcctcggcgtcctcgtccccctcggcgtcctcgtccccgcacccttcggccgccgtgcccgccgccgatcccgccgactcggcctcaggcagcagcaacaagaggaagcgcgacaacaaggccagcacgtatggactcaactacagcctgctgcagcccagcggagggcgggccgcggggggcggccgagcagacggcggcggggtcgtgtacagcggtaccccgtggaaacggaggaactacaaccagggagtcgtggggtgagtgctggccctgcggcccgatggcctggccggtgcgaaagcgcagccgagcacacgcccacagtggggggttgtgagggtctgggagcggccacccccacggcctgcctttgcttctggtgcacgggggtgctgctggccatccccacccccctagtcgtccacacctttccccagcctccttaaccgtccccaccctccgctctcctgtcctcccttagtcgtccacaccttcctcccctccctcttaaccgtccacaccttccccaggtcccccctttatccattcactctcctcccatcccccttagtcaaacacatctacccctgaccaccaccccgcctccagccctccacacctttttccccgtcatcacaactcaagatgagaccgcttaacacgggcatatcattcattccctgagaacattggtgtgtgagtgttttttgatggtgcaggacccggaggtgctttccttgccaagaatagaaacatccagaatgctcctccccctcccccagtcccagacagcaatcatgtcagccctgtaaggcattgcctgctcttgaccctttggccaatctttttattttttaaaaattcgcatatcacagatgccctgtctgtggagagggtggcgtgggatgggtgaccgctaagtttaggctggcgaaggtggtgagctcttctgaggccctgatagaactttccaggagttcatggtccgcggctccagcttctcactgtaaagttgtcatcctggcagaggcagccaatgcttttcattctagggggtagagatttatgctaatgagtgaatattgcaccactagtgactgtttaaagttcagctgttagaaaatggaatcttacctgacccctagtgaattatgtacataagcagggaatgtttccagctagatcacccttcagaagagtccctgtgttggaataggttactgagtcttatttgttttgcaaaacaaagcttttggtctcgtgtgtgtgtgtgtgtgtgtgtgtgtgtgtgtgtgtgtgtgtgtgtagcttgagtatggagaacgggctttcaaattgcttttcatttttcaggttctgttttacactgagggctttggcatgcaaatgaaattaccaattagtaatctcatgtgaaccttttcctggatttattcattcagatctgtcctgctttggctgagagagagagttctgtgtacctttttgaaggtctggataaaatgagttggtgggttccatctgcttccagtgggctggtgtctgctctatgctactattacaactcctaccttttgtggaaaatgcagtcaagcgttttaggactggtgctgtggtacatgtcaaacctgccctcacattccagaaagggaacccttttagggttgagtcctctgttgctaagcttcaagggcgctctccatggtcatcacgttttattaaaggcttgtggttccatcctgttagcatttccaagtctaagcgtaaacctgtggtttagtgacaagcaaattgatgttgagggtttctggtagtttcatttcacaggagtaagctccagttaagtaatcactgtcaacgaaaaccttgaagttccttaattgcattttattgaagcctccttgcatgtgtctagcaaaagatataagtccaagatgcttatttttttttgataaattagaaattgtcctttcctctgcttgctatttaatgcagaagatactctaaaaggttcatatttgtacctagtagaagcaagatgttcttgttcctaattcaaatatattgccctcaaagggattaggagaggaattttcatttcccagagggattactgttttaaaactgattgtaaacctctttaaaaactgcttatcacttcaccagtttttccattcttttgcctcctcccttagaggatgtcagcagttaattttttaaaaaaaaaattgaaaaaagaatttcaattctgagtcctcctagtttcaaaaaatacgttaaacaattcccaggagtgttaagagtgtcggggtgcttagaaattcttgctttgattcatgtatcctgatttcttttttttttttttttttttgagatgaagtttcactcttgttgcccagggtggagtgcagtagcacaatctcagctcgccacaacctctgcctcccaggttcaagcgattctgctacctcagcctcctgagtagctgggattacaggtgcttgccaccatgcctggctacttttttatttgtagtagagacggggtttctccatgttggtcaggctggtctcgaactcccgatctcaggtgatctgcccgccttggcctcccagagtactgggattataggcatgagccaccgtgcccggcctcattatcctgatttcttttttttttttttttttttttttttttttttttttgagactgggtctcactctgctgcctaggctggagtggagtgatgtgatcatagctcatggtatcctctaactcttaggctcaagtgatcctcctgcctcagcttcctggagtacgtgggactacaggcacatgtcaccacacctgcctaatttttttatttttactttttgtagagatggggcctccatttgttgcccgggctggtcttcaactggcctcaagcactcctcctgccttggcctcacagagtactgggattataggcatgagccaccattcttgccagtgtccttatttcttaaggaagtttttctgtttttgatacaggtatttcaaaatatctgaattcagagtgcacctcgatgttttgctgttctgagatttaatatactaaaactattaccattgttgtctgaattcttaagatgtgactgatagttagctaataggttaacacgttgtgttggttcttggcctctgaactgatagtccagatagggagaggacaccagaaagcatgtgaaaaatggactagaactatggaacagctatatagtctctcacagctgtcttttgtgttctctgcttcaaccaaactggttgacttatttagaattctgacctcttgcattgcctaagtccttgatgtttttggtttcttctctgaactctcaaaggtactcacttcatgctcttggtatagcccacttatgtttaactttccttttattatgtcttccctcttacacatgacatggacatttcttttaatatgtagagtaagatattggatttcatctaaagtcttcaaaataaaactcttgggctcaccatctcagacttcttcatgtatttacagaccagggattttgtctgctttttaaaaaaattttatattttttattatttttaaattttaatttaattttatggagacagggtctcgctctcttgcccaggctggagtgcagtggtgtgatcttgactcactgcaacctttgcctgggctcaacccatccacatgccttggcctcccagagtgctgggattacaggtgtgagccactgtgcctggcctaaatttatttttttaattttttttgagacagggtcttgctctgtcactcaggctggagtgcagtggcatgatcatgggtctcagcagccttggcctcccaagctgaagtgatcttcccacctcagcctcctgagtagttgggattacaggtgagtaccaccacgcctggctcattttggtattttttgtaaagatggggtcttgccatgttgcccatgcaggtctccaactcctggcccaagtgatcctcccacctcaccctcgcaaaatgttgggattacaggtgtgagccactgtgcctggccttatgtatttatttaattatgaatgaatgaatgagagggagtcttgctctctcgcccaggctggagtgcggtggcacaatcttggctcactgcaacctctgcctcccaggttcaagcagttctcctgcctcagcctcccgagtaactgggattataggcgcctgccaccatgcccagctaatttttgtatttttagtggagatggggtctcactatgttggccacactggttttgaacttctgacctgcccacctcggcttcttaaagtgtcaggattacaggcatgagccaccgcgcctggcctggccttttatgttttaagttgcttccactgattctctttcttgggctttgctgccctccagaactggctatggtgtaggatgctgtccacctgctgctgcttgtccatgaaaacgagccataaacccttttatttggaaagacttagttgttgatcgctatggagaaagaggggatggcaagaagtagcaactacagagaatttgcagaacttggtcttgagccctgggtccagaaacttcttgtggaaggtgcttggtgtttgtccaagctcatgaggataggtttctgttggctgtactgccagatctgtagatgcttttttaaggcttggatgacttgttcaaaacaacgttttggagtacaaatttggcttggggacatcaagaccttgttgggaaacttgggtttaagatataatttcttaaactaggatggtgggaatggggatgtgaagggagaatgaatgtgagaggcattacagggtaaggatggagaggattcagattccttaagtggatttaataatcacactgtagctttgaacttcagtgactggggaaatgtttgtggtgtttttggaaataagggccagaaggactattggtttggggaagaagatagtagggggatatataggtagacccgctagtggggtgctgagatttggagggcagagatgtagtgctcttcactgctgtagggcagtatcgtcctgtatgtgccatcctctagtgcccttttttcaccatattgtagtaagcccgagatgttcattcctttcttcagtactgcattaaggcttatctctgcttgtttctttgtttctgtgttttttttttttttttaagatggagttttgcttttggtgcccaggttggagtgcaatggcgcgatcttggctcaccgcaacctctgcctcccggtttcaagcgattcccctgcctcagcctcccacgtagctgggattacgggcatgcgccaccatgcccgactaattttgtatctttggtagagatggggtttctccttgttggtcaggctggtctcaaactcctgacatcaggtgatctacccgcctcggccgcccaaagtgctgggattacaggcatgagccaccacgcccggcctatctctgcttatttctgcacagtattatcagtgagattggtgttactgctgggctccaaagcaatcagacatagtaaagtagattgaatatgaaataatttagaggccttgttccaagtgatttgtgctttgtttaatttctgtgcatttgtaaatatagcccacagtaattcttagtgaactggaaccttcaggttattgcattttactgatttgggtactgaaatgtgcttttaagaagacattaggttttctatagtgtagattgtacactaacaatataattcatatttaagaatgtctcaaaatttagtatgctgtgttcagctaacttaactttatttgtttttttgttttgttttgtttttttctttgagatggagtcttgctgtgccacccaggctggagtgcagtggcatgatcttggctcactgcaacttcaacactcctgggttcaagtgattctcctgcctcagcctcctgagtagctgggattacaggcacccgccaccacaccagctaatttttgtatttttagtagagatggagttttgccacgttggccaggctggtctcggactcctgagtcaaatgatctgcccgccttggcctcccacagggctaggattacagacatgagccactgcgcccggccactaacttaactttcattccacaacttccatcttttatccaaaatctgtgatcagtgaacactgtcaccattaaccattgacatttcagtgtttggacttttttttttcttttccccctttgtctttgtggactcttttttaacactcataaagttttaactattgaaaagcacaaagaaacagtgagtgactttttggaatttgtttacccagtgttcacataaaaggcttactacattacaggaaagataggatgagaaagggatactagaaaattctaagtcagggacgggggtgtgtattagaaaaattctgatcctggcatgccagatggccttacatctcaacttcttctgtgaaattcctgccaacaaatcatagtgttggaagtacagaagggtccatgggaacagaatttaagggctcccttggtgatactgaactgatcagatggttctcacttgttctcagataacctgcatactgaatatcacaggaagggtatagacatcatggctatggttagatattcttgcacctgctgaagctgagaaaattaaagtcatttttttttctgtggaaagtagaaaatcaagcttttatatgatttcacacagctttctattctctcttctgttgactctgttaagagtaacatttagtggtggaaactatttcaggatcacacccacaacactagagactgtattaatcactcacacacacataggtatagagtaatcttgaaggggctgtaggccagagataatgcttttttgaagaattagagactagttaccagcacctggtatttgctgtttcctacagacctgactggacagcttagagtctgctgaggaattcagaggatggccagtagaatgttctttctaccccagagtatttggtagggactcagctgctatggaatgccaaaaaggctttaagtttctttcactattcttaagattacatgtaattgtttttttgtaagagattatatatattcaaattgaggatggctttgagctagacttttccttaatttggaaccacacagcagatgatacatttatttccatctgataagttacttgatgatgtaaaagacatttgagttaaagatttttgggaaaaaagctgaatgttgagccatttatgttgtgtactggttccctattcacttagacaattttaagtctgaaaacaatcttatcatatgcacaagagatttcatgtagtatttggtaattaagttgaggaattctagctcaagtcatgctttttgctgaaataggtgtatatatttagtgcaaaaacctgtgttttcaaaaaaaattaatgtaataaaagtttcaacaaaatggaagcctttataaccatgtttcaaatgctatactaaaccttttccatttgttattatattaacctcctcatacatagccctactatttttttttttttttactttctattttgaaataattgtagatttatgggaagttgtgaaaaatagtacagagcccatgttcccttcaccaagtttcacctggtggtgatagctcacataactatagtttaatgtcaagaaccaggaaattgtcattgttacaatccataaggcttttttagatttcaccagtttcacatgtatttgtgtgtatgtgtgtatataatagtatgcaattttatcatgtgtagatctgggtagccactctaacagtctatagttctatatacagaactactccatcacctcagggctccccatgctaccgctttgtagccgcatgcacccttccaacaaccactaatctgttctgcatctctgtaattttgccattttgagaatgttatataaatggaatcatacagaatgtaactttggcttttttcttttaccataattcctttgagagccatccaaattgctgcatgcatcagtagttcatttctttttactattgagtagtagtccatagtatggctgaaccacacaatttgtttaaccatttacttgttgaaggacataccagaagggtggtttccagttttttgactattacagataaagctgctataaacattcatgtatataaatatttttatatgaattaaagttttcattttgggggaataaatgcccaaatgtttggatcatctggtaaatgcatgtttggtttttagagaaactgctgaactatttattttctagaatgactatatcctcttgtattcctatcaacaacgtatgagatatccagtttctctgcatccttgctagcatttagtgttaccatttttttatttgagcggttctaatatgtgtagtaatagcctgttttgcgttatattaatcaataaaaatagcctcatctaattttaactttttaattttaaaatatcttggtggtattgaactttctcagtgagaaatatctaaaattgtgacttgaaaggctttaattttcaagtttttctttggttttactcttagcagtaacattttaattttttttgtctttgaagtaattttcagtgtttcctttacatgttgctttttcttagaaactagttattagcatgaagtagatctttagtctcgttttctaaaaacataaaaaagtaaaactgcgggatttatttcaaaattgagagtcttgtcttttcatatgaggatattttatagtctgttggcttggctatattttagggagtaaacctgtggctagtggtttgttggtgatggtggtggtaaagttttcttacagaatttttatttttttttttattttttattttttatttatttattttttttgagacggagtctcgctctgtcacccaggctggagtgcagtggccggatctcagctcactgcaagctccgcctcccgggttcatgccattctcctgcctcagcctcccgagtagctgggactacaggcgcctgccacctcgcccggctaagtttttgtatttttagtagagacggggtttcactgtgttacccaggatggtctcgatctcctgacctcgtgatccgcccgtctcagcctcccaaagtgctgggattacaggcttgagccaccgcgcccggccaagaatttttatacctgaatgatacctttagactctatagaatagatacttgatttcaaatctatcctagaataaattgtttcatctaaacagctttgtgacctgagaattgggacttagtgccttagttttcccttactggccctttgtagtcactgttttgatttagtcaaagtaacctaactcttagcactgtcaggtattgtacattcctgccaaagcaagaataataatacataggattgtgttttaattctataattaggtgacttagctaatttccaggaacttggacttaatacagtactagtgataaggcttgaaattttagtgcctttgttctttgaagttattcaccctttagtttcgtgtttgtttggggtgtttataccactgtccctaaatatagctgaaataacggaggaaaacccctgtaatgtcactagcaggatataatttctgttaatagtttgatgtaaatattttttgactttttaatttttttatatatatatatattttctttcatcaatatggactcttactgtgagcataattttaatgtctttaaagagttgggttttgtttatttgtttattttattttatagaaatgggatctcactgggtgcggtggctcacgcctgtaatcccagtactttgggaggccaaggcaggcgcatcacctcaggtcagtgaggttggtcaccagcctgaccaacatggagaaaccccgtctctaccaaaaacacaaaattagctggacgtggtgacatgcgcctgtaattccagctacttgggagcctgaggcaggagaatcacttgaacccaggaggtggaggttgcggtgagtcaagattgcaccattgcattccagcctgggcaacaagagtgaaactttgtctcaaaaaaaaaaaaaaaaaaagaaagaaagaaagaaagaaagaaatggggtctcaccattttgatgggttttgaacttgtggtctcaagcagtcttcccacttcagcatcccaaagtattgggattacaggtgtgagcccatcctggttttttgttgttgtcgttgtttgcttgttttttgtttgccaccgtacccggctaatttttgtatttttagtagagacagggtttcaccatattggccaggctggtcttgagctcctcacctcgtgatcttcccacctcggcctcccaaagtgctgggattacaggcgtgagccactgtgcccagcctattgttgtttaaataaaagaatgttgtttaaataaaataaatttattctcttatagtctggaggccagaacttagaactggttttcagtctaattttttttttcttctttgggagaagggcatcagaatattgtgaatatacttttttgactaaaaaaagttttctctgggcatggtggctcacctggaattgcctgtaatcctggtactttggaggaggctgaggcaggtggatcgcttgagtccaggagttggagagcagcttgggtgacatggtgaaacccagtctctaccaaaaaaaaaaaaaaaaattagccaggcatgatggtggcgtgcccttgtagtcccagctacttgggaggcttagctgggaggatcacttgagaccaagaggcagaggttgcagtgagctaaattcataccactgcactccagcctggatgacagagcaagacctcgtctgaaaaaaaaatttttttttttactagcatgacaaacatcttttcatttcaaatatatttctttttttttttttttttgagacggagtctcgctctgtcgcccaggctggagtgcagtggccggatctcggctcactagttttttgtattttttagtagagacggggtttcaccgtgttagccaggatggtctcgatctcctgacctcgtgatccgcccacctcggcctcccaaagtgctgggattacaggcttgagccaccgcgcccggcctcatttcaaatatatttctataccatttttaatatctcattgcctttagaatgaccttgtattcatagtacatatgtatgtgatattcgatttatttatttttcgtttgtcttatttttggttatattccattgatttaatgtaccataatttatcttaccaatttcttgctgaccattttgtttccagtcttttgtttttttatcagacatggattaagctgaggctttgtcccagacaacattatttcttttttatcagcaaaatatgcatgtaatgaaattaaaattaaaaggcaaaaaacgttatcctttattttcttcttatttttgttgagataataattcacataccataaatttaacccttttaaagtgtacaattcagtggttttgtatattagaaggttgtaccatcacaactaattccagaacagtttcaagaaactctgaacccattatcactccccactccctcacacgccctaatcctggcagccacatagagactgtctgtctctgtgtatttgtctattctggacctttcatataagtggaatcataacaatttgtggccttttgtgcttggcttctcaaacttagcacaatgtttgcaaaggttatctgtgtcgtagcatgtgtcatacttcattcctttttgtgactgaatattttattgcatatatatgtcacattttgtttatccattcacctgtagaaggatttttaggttgtttccattttttagctgttatgaatattactgctgtagacgttcatgtacaagtttttatgtgaatgtgttttcatttttcttgggtatatacttaggtaagaaattctgggtcttatgttaactctctgtttaacattttgaggaactgtcaacttgttttttaaagtggctgtgacattttatattctaccagcagtgaattaaatttccaatttctccacatacttgacagcattttttgttttgtttttttttttgttttgttttttttgagacgaagtctcgctttatcgcccaggctggagtgcagtcgcacgatcttggctcactgcaacctccgcctcctgggttcaagtgattcttgtgcctctcagcctcccaagtagctgagattacaggcacgtgtcaccacacctggctaatttttgtatttttttttttttttgagacggagtctcactctgtcgcccaggctagagtgcagtggcatgatctcaggtcactgcaagctccgcctcccgggatcacaccattctcctgcctcagcctcccgagtagctgggactacaggcgcccgccaccaccccgggttaattttttttatatatttttagtagagacgggattttaccgtgttagccaggatggtctcgatctcctgacctcgtgatctgcctgccttggcctcccaaagtgctgggattacaggcgtgagccactgtacccggcctaatttttgtatttttttttagagacaggattttgccatgttggtcaggctggtcttgaactactgacttcaagtgatccacctgccttagcctcccaaagtgctgggattataggcgtgagccactgtgcccggcctacactttttaaattatctgtcttctttattatagccaacctagtgagtatgaagtaagtgtctcatttgtgattttgattgttagtggtgactaacaatattgaatatctttacatgagcttgttggccatgtacacatctttggagaaatatctattcaaatcttttgaccattttaaaattgggttatttatctttttattgttgagctataggagttctattttattttattttattttattttattttattttactgagacagggtcttgctttgtcacccaggctggagtgtagtgatgccatcttgactcactgcagcctctgcccccaccccaggctcaagcaatcctcccacctcagcatcccacagctgggaccacaggcgcatgccactgtgcctggctaatttttttttttttgtattttagtatagacggagcctcaccatattgcccaggctggtctcaaactactaagctgaagcagtctgcccatctcagcttcccaaagtgctggaattagaggcatgagccactgtgcctggtctattttatttttaagatgagactttactttgtcacccaggttggagtgcagtagcatgatcatagttcactgccattttgccctcctgggctcaaatgatcctcccggcttatcttactgagtaggtaggactgcaggcatgtcgctaccacgcccagctaaaagagttctttatattctagatcggggtatccaatcttttgacttccatgggccacattggaagaaaaattgtcttgggccacacataaaatacactaacagtaatgacagctgatgagctaaaaaagaattaccaaaaaatctcataatgttataagaaagtttacaagtttgtgttgggccacattcaaagccatcgtgggcctcatggctgtgggttggatgagcttgttctaaatgctagacccttatcagatggatggtttgtagctatttatctcatgctgtggattttttttactttcttttttttcttttaggtttttttttcttaaataattcaactgattataagctttaatcgttttgttttcttgatagtgtcttttaaagcacaaagttttatttcgatggtgtctaatttgtctgctttttctttggttgcttgtcatatgtaagaaactgttgctaaatccagaatgctgaagatttacttgtgaactttgtttccttctatgtgttttatagttttagctcttacatttaggtctttgatcattttgggggttttttttttttttttttttttttttgacagagtcctgttctgtatcccaggctggagtgcagtggtgtgatcttggctcactgcaccctctgcctcctcggttcaagcaattctcatgcttcagcacccgagtagctgggattacaggcatgtaccaccaagcctggctaagttttgcatttttagtaaagagagagtttcaccatgtttgtcaggctggtctcgaactcctgggctcaagcagtcctctaaccttgacctctgcaagtgctaggattacagggtgtgagctaccgcgcccagtccatttggagttcatttttaaaatacggtgtgaggtagaggtcccatttcattcctttgcctgtgggtatccagttgtcccagaaccatttgttgaaaagactgttgtttctccattgagcaatgttgtgaggccctatctccataaaaaaaaaaaaaaaaaaaaagaatgcagaaggaagcagtttttgccaattttgtagtatttactgacaatttgcatatatctgtacattctttagctatttatttttcttttgagttattgcctttgttcatttttcttttggaggtgtttgtctttttcttattaatttgtaagagattttgcaaatgtatacaatttcttttttttttttttttttttttttttttgagacggagtctcgctctgtcacccaggctggagtgcagtggcgcaatctcggctcactgcaagctccgcctcctgggttcacgccattctccggcctcagcctcccgagtagctgggactacaggcgcccgcccctgcgcccggctaattttttctatttttagtagagacggggtttcaccatggtctcgatctcctgaccttgtgatccgcccacctcggcctcccaaagtgctgggattacaggcgtgagccaccacgcccggctaatttcttttcttgtttgttttgagatagagttttgctcttgttgcctaggctggagtgcaatggcatgaccttggctcactgcagcctctgcctcctggtttcaagagattctcctgcctcgaccttccgagtagctgggattacaggtgcccaccaccacacccagctaatttttttttttttttttttttgtatttttagtagagattcgtttcatcatgttggccaggctggtctcaaactcctcacctcaggcgatccacccactttggcctcccaaagtgctggaattacaggtgtgagccaccgtgcctggacctcccaccttattttgaaacaaatttctttctttttttttcttctttttttgttttgagaccaagtctcgctctgtcacccaggctggagtgcagtggcatgatctgtgcttgctgtaccttctgcctcccaggttcaagtgattctcctgcctcagtctcctgagtagctgggattgcaggcatgtaccaccacacctggctaatttttatatttttagtagagacggggtttcaacatgttggccaggctggtcttgaactcctggcctcaggtgatccacccgctttggcctcccaaagtgctgggattacagacttgagccattgcgcctggccagtatcacataatttcatataaatatttctttgtgtatctttagataaggacttaaaagaaggcataatcataacaccattattaatacctaaaagaaatgaataataaataattcatttgttgtatcaaatatccaatgttcatatttcctcaattgtcccataataatttttaaaagtttgcttaaatcaaaatcgaaacaagattctttcattgctattgtttgagatacatttgaaatcttaatttatagatttctctgccatttcttttcccccatatttatttgttgaagaaatcaagtgttgtttcctatggactttcttactatctggattttgctggttatattcttctggcgtcagtttactatgatcccttttcccctgtattttctataaatttgtaattaggtctagagatttgttaagattttgtggtgtttttttttttttttttccaaaaatgcatcataaatggtggtgtgtagatatcgcagaagacacgtatcttaatgtctttttgtggtattagtagttattaatgattactacctatatttattaattcattatttggactataagtttataatattctcattctcttgatcctttttctgttgttagctgtaatgcttctaaaaggagaaacctttcctcttcaagctacttggttgtcttaagggttcacttctaatagggaaaacgggctaagggtgaaaaaggaaatagtttttaactgaatctgttaatgagctgtcaccccaggcaaagagaagcaaggcaggccctaggaaactgaagtgggtttgggatgattggtgccccatgcgtgcatgcatgaagggaagttaatcctccctgtagtgaactctacttggctttttgtcagtggccaggactgtcaaggaagacctttgtccaaagtcatacctggcctttgcttttagctcttggtagctgaaggaaaccaaaacagacctatgacctgcaaacttctgcttagtagacaaagttctcacagcctcaattcagtaagcaggagtaagatgcttgctttcccttgaagtgaaatgtgaattacatgtttcttcaacttgtgctaatattctcttttttaatatttatttatttatttatttattgagatggagtctcacactgtcgccgaggctggagtgcagtggtgcaatctccgctcactgcaacctcagtctcccgagtagctgggattacaggtgcctgccatcatgcccggctaattttttgtatttttagtagagatggggtttcactatgttggccaggctggaattgaactcctgacttcgtgatctgcctgcctcagtctcccaaagtgctgggattacaggcgtgagccaccacacccgggcaacttgtgctaatattcttaacagggtgtgaatcattcctgccctcaggctaacataacccatacagccttccttataggaagatttcctactgggagtgaatttgtccagtgattcccccaaaatgtcccccaatcaaatattttaaaactcagcatttacatgtaaaaactaacaagcatggtagcagcagtataaagaaacagcagtatggcccttgtaagggaaggctccggaagatgagctgcactcagcctctaggtcacagctaccttaggagtttgcagtagttcctggggaagtcagtagacaggccatctctcaggcctgggcaagatagggacttttttttttttttcttttgagatagagtctcaccctgtcgtccaggctgaagtgcagtggcatgatctcggttcaccacaacctccacctcctgggttcaagtgattctcctgcctcagcctcctgagtagctgggactacaggcgcggacaaccatgcctggcccatttttgtagagatggggtttcatcatgttggccaagctggtctcaaactcctgacctcaggtgatccacccccctcccaaagtgctgggattacaggcatgagccactatgcccagtgttgttttttttttttttgaattgtagtgataggatctcactttgtttgatgggctattcccaaactccaggcctcaagccgtcctcccgccttggcctcccagaatgctggggttacaggtttgacccactgtgcctggtcccagaattcatgtttttaaaagtcactctgtgccaggctcatgcctgtaatcctaatactttgggaggctgaagcaggaggattgcttgagcccgggagtttgagaccagcctggacaacatagcaaaaccctaactctacaaaaatacaaaaactagccaggtgtggtggcatgtgcctgtagtcccggttgctttggaggctgaagtggaaagatcgcttgagtctaggaagttgaggtttcagtgagctgtgattgtgcctgggcaacagagtgagaaaagtcactctgattgtggtggggaaagtggattaatggagaatggaagctgggaaacatggtggttcttgctaagtcagtatcaagggttcacagatgagggggctgtttcttcctagtaagggccttggccaaatagtcatggatctttctctttggaagaagctcctcaatttttcttccccaaggcatatatggttgatgcttgaacaacacaggctgggtctgtatgggttcacttatatgtggattttttttcaaccaaacttggattaaaaatatagttgtaggccagggtcagtgactcatgcctgtaagcctagcactttgggagcccaaggcaggtggatcacctgaggtcgggagtttgagacgagcctggccaacatagtgaaaccatgtgcctactaaaaatacaaaaaatagctgggcttggtggcgtgcgcttgtaatcccagctactcaggaggctgcagctggagaattgcttgaacctgggaggtggaggttgcagtgagccaacggggtggaggttgcagtgagctgagattgcaccaccacactctagcctgtgtgacagagcaagactctgtctcaaaaataaataaataaaaatacagtgtaggccaggtacagtggctcatgcctataatcccagaactttgagaggccaaggcaggcagatcagttgaagccaggagtttaagaccaatctggctaacatggtagaaccccacttctactaaacagaagtacagaaattaaccaggcatggtggtacacgcctgtaatcccagctacttgctaaactgaggcaggagaattgtaatcccagctgcttgggaagcttgaggcaggggaattgggaggcggaggttacagtgagctgtgatggtgccactggactccagactgggtgacagagcgagactctctctccaagagaaaaaaaaaaaattgtatttacaggacatgaaacctgcctgtatgatgtgctgactgggagactagagtatgcacagattttggtaaacaaggggattcctgaaaccaatcccctgagtatatggaggattgactatatattttaatagaatttatttattattatttttagcagttttaggtttgtggaaaaattgagcaggagtacatagtttctctctctccctcacatttccccgttactaacgtcttgaaatagtgtggtacatttgttaaaactgaagagccaagtattgatacatcactgttaaccaaggtccatagtttagagttcattcttggtgttgccgtttctatgagtgttggcaaatatataatgacatgtatctaccattatagtatcatattgagtagtttcactgccctaaaaatccccctcgttctaccttttcatccctccctctatctacctgaacccctggcatccattgatccttttactgtctccatagttttaccttttacagaatgtcaaatagttggaatcatatagattggcttctttccatgttccttctggcttgatagctcttttcttttctttttttttgagacagagtctcgctctcgcccaggctggagtgcagtggtgcaatctcagctcactgcaaactctctgcctcctgggttcaagcaattctcctgtctcagcctcccaagtagcttggactacaggtgcctacctccctgcctggctaatgtttgtatttttggtagagacggggttttaccatattggtcaggctggtctcaaactcctgtcttcaggtgatccacccacctcagcctcccaaagtgctgcgattacaggtgtgagccaccctgccctgccagctcttttctctgtactgctgactaatactctattgtataggtgtatgagtttattcaccttctgaaggacattttggttgctcctaagttttggcaattatgcatgaagttactataaacgtctgtgtgtaggtttttgtgtggtcatgtttttagctcatttggataaacaccaaggagcacgattgctggattgtatggtaagagtatgtttagttctgtaagaaactgccaaactgtctttcagggtgactgtaccattttacattcccaccagcaatgaatgaagttcctgtcgctccacatcctcgttagcatttggtgttgtcagtgttttggcttttcaccattctaatagatgtgtagtgatatcttgttttagtttgcaattctctagtgatgtatgatgttgagcatcttttcatctgcttatttgttgttgttgttgttgttgttttttattgagatggagtctcgctctattgcccaggctggagtgcagtggtacaatcttggctcactgcaacctctgcctcccgggttcaagtgattctcctgcctcagctccccaaggagctgggtttacaggcgcccgccgccatgcccagctaatttttgtatttttagtagagatggggtttcaccatgttgcccaggctggtcttgaacttctgacctcgtgatctgcctgagctcaagcaatccgcctgcctcagcctcacaaagtgctgggtttacaggcgtgagccaccgcgccctgtcttcatctgcttatttgatatgtgtatatgttatttggtggagtgtctgttctgatcttttgcccattttaaaatcagattgttttattgtttctgcggttcttttagtttgtttttttgagacaaactctcgctctgtcacccagtctggagtgcagtggcgtgatctcagctcattgcgacctctgcttcctgggttcaagcgattctcgtgcgttagcctcccaaatagctgggattacaagcatgtgccactgcacctggctaatttttgtatttatagtagggacagggttttgccatgctggccaggctggtcttggactcctggccttcagtgatccacccacttttgcctcccaaagtaatgagattacaggtgtgagccactatgcccggcttattgttaagttttaagagttctttgtatgtgtgtattttttgtgattcttttaaagttaatgcttaataaaataattgtacatatttatgggatgcatgtgatattttgatacatgcatacaatgtggatcaaatcaaggtaattagagtatcacctcaaacatttttcatttctttatgttggaaacatttcaaatcttctagctattttgaaatatatagtaaattattaactataagtcacctcattgtgctatcaaacattagaacttattctttctacctgactttattttttttacccattaaccaaccattcttcatcagctccccatctcccctactctttttttttttttgagatggagtcgctctgtcacccaggcgggagtgcagtggcgcgatctgggctcactgcaacttccgcctcccgggtttaagcagttctctgcctcagcctcctgagtagctgggattacaggcgaatgccaccatacctggctaatttttgtatttttagtagagatggggtttcaccatcttgtccaggctggtcttgaactcctgacctcctgatcacctgcttcggcctcccgaagtgctgggattacaggggtgagccaccacgcctggcccctcttactctttgcttagcctccgatatctatcattctattctctacttctatgagatcaactttttttttagctcccacatatgagtaagacatgtaatattcctctttccgtgtctgatttctttgtatattttggataatagtcttttattagatacgtgttttgcaaatattttttcccagtccctgacttatcttttcattctcttaagtagtgtcttttgcagagcacacattttacattttagcacagtccagtttaccaattccttctttcatggattttgcttttggtattgtgtctaaaaagtctttgccaaaccacagtcagctagagttccccttaccttacaggatttttaagtttttgttttacatttatgtctgtgattcattttaagttaacttttatgtgaaagatgtaagatctatgtctggaatttctctttttttttgagatgcagtctcgctctgtcgccaggctagagtgcagtggcgtgtgatctcggctcactgcaacctctgactccctggttcaaatgattctcctgcctcagcctcctgagtagcacacgccaccatgcccagctaagttttgttttttagtagagacgggatttcaccatgttgacctggatggtcttgatctcctgacctcgtgatccgcccgcctcagcctcccaaagtgctgggattacaggcatgagccaccacgccctgccgggcttaaccatttttaagtgtacagttcagtagtgttaagcacattcacattgcaaacaatctctacaagtttttcaaaaaaactgaaaaacttgcaaaactgaaactttatacccactaagtggtaactccccattctagtggttaccctagcccctggtaaccaccattctcctttctgtttctatgaatttgactactctgggaaccttagataagggaaatcatacagttcttgtttttttgtggctagcttctttcacagcatgatgtctaaggttcatccccatagtaccatgtgatcctaatgtttttaaacctccacagcaacactttcaagttagatttcacattttacaggtgagaaaatggagactcaaaaagaaaaagcaattgtccataggtagtgtcatgggttgacactagctttgaaggttggtccccacctccaaagacatgcccaagtcccaagtcccagtaccgtgaatgtggtgttatgtggaaataggtctttgcagatgtaattaagttatggatctcgatatgaaatcttcctgagtttggggtggactctaaattttacgactgatgtcctcataagagaagggagggggaggtcaggcgcagtggctcacacctgtaatcccagcactttgggaggctgaggcgggcagatcacctgaggtcgggagtttgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatggagtcttgctctgtcacccaggctggagtgcagtggtgcaatctcatctcactgcaacctccacctcctgggttcaagcgattcttctccctcagcctcctgagcagctggagctacaggcatgcgccaccacgctcagctaatttttgtattttttgtagagatggagtttcaccatattggccaggctggtctcaaactcctgacctcaagtgatccacccaccttggcctccctaagtgttgggattacatccatgagccgctgcgcccggcctataattctttatgtacattgttagattcagtttgctagtattttatttagcatttgtctgtgttcatgagaggtattgttctgtagttttctttggtttcttttctgtctagtttagggtaatgctggcctcatagaataggttaggaagtacttcctctgcttctgtttctgaaagagaattgagataatatctttttttttgagatggaatcttgctctgtcatctaggctggagtatagtggtgccatcttggctcactgcaacctctgcctcccaggttcaagtgcttctcctgcctcagtctcctgagtaggtggaattacaggcatgcaccaccatgcctggctaatttttgtatttttagtagagatggggtttcactgtgtgggccaggctggccttgaacttctgatctcaggtgatccgcctgccttgtcctcccaatgtgctaggattacaggcatgagtcactgcgcctggcctgagataatatctaaaacagtttggtagaattcaccagtgaacccatctgggcctggtgccttttgctttagaagattattgattattgattcaatttccttaatagataaaggtacattgagattgtcttttcttcttgggtaagttttaatacattgtgtctttcaagaaattgttccatttcatctaggttatcaaatttgtggattagagtccttcataatatttctttgttttgcttttgatgtccacaggtttagaagtgatggccctttttcattttttctattagcaatttgtgtctttgccttttttttttctttcttaatctggctagaagcttatcaattttgttgatcttttcaaagaaccagtttttggtttcactgatttttctctgttaattttgttttcagtttaattgatttctgttgtaattggttttcttctgctcactttggatttttttttagttttcctagaaaactaagtttttaagtgaaaactgagattattgatttttagatcttttttgtaatgtttatagttaatgctatacaatttcctgtaagcactgctttctctgtatcttacaaattttgataagtcatattttcattttcatttagttagacatatctcttgagacttctttgatccatctgttatttagaagtgtggtgtttaatctccaagtatgtattttgggattttctggctatctttctgctattgatttctagtttaattacatgtggtcttagagcataccttgtttgctttctattcttttcaatttgttaaggtgttctttgtggctcagaagttggtctacttttttttttttttttttttttttaaagaaaaactggctagatgcagtggcttatgcctgtactcccagcactttgggaggcctaaatgggaggatcacttgaggtcaggagtttgagaccagcctgggcaaaatttttaaaagattagttgggtatggtggcatatacctgtgtatggctgaagtgggaggattgcttgagccctggaggttgagactacattgaactatgatcacatcactgtacttcagcctgggcaacagagtgaaactttgttctctcttgaaaagaaaaaaatagttgatgacataaagttcattcatcttttttgtatgtgacttcaaaataactactgatggttaaaaaaaaaatcagaatgatgcagcccaagtgtccatcaatggatgaatagataaacaatatgtggtgtgtgaatacaatggactactattattcagccttaaaaaataagaaaattctgacactgctgtaacatggatgaaccttcagctcgttatgctaaatgaaaaaaaccagacgcaaaaggacaaatattgcatgattgcacttatatgaggtgtctggagtataaaagtcatagaaacagtaattcaataattagaataatgattgccaggggctctggggaggagggaatgaggaattcatgtttaatgcatacagagtttcatttggaaaagattaaaaagttacggaggtggatggtggtgagggttgcacaacagtgtgaatgtacttaataccactgaattgtacacttaaaaatgattaaaatggtacattttatgttacatatattttacaacaacttttacagatggaaaaaatttattaaaaaacatcaggatggtgttgacagtgaaaaggttaaagagttactttaaaaatttactttattccggccaggtgccgtgactcacacctgtaatcccagcattttgggaggccgaggtggacagatcacctgaggtcgggagttcgagaccagcctgaccaacatggagaaaccccatctctactaaaaatacaaaattagccgggcgtggtggtgcatgcctgcagtcccagctactcgggaggctgaggcaggagaattgcttgaactcgagtggcggaggttgcagtgagccaagatctcgccattacactccagcctgggcaacaagagcgcaactccatctcaaaaaaaaaaattttttttaattttattccaaatatttatatttactttggggcttatgtgaccagtttaattttcatttgtaattgacttgatagaatacactatgttcagttaagattccttatggtgtgatgaggaataggaattttatgtaagtaagcccaaaattgtatcagattaatctgatatttgcagaagatattcatgcattaatgtaaggaccactctgcttattcatttgactgatttgtagcaacatggttagaaatcatcaaggtgtttgagatcaaaggatcatcagaggtcatttactctaatcctttctttaaaaaattaataacttgagcctcagagaagttaaatgatattaccaagttctgctgttagtatagtgactttatctttacctgaagccagggcttcttagccctagtctgtaatgtgtcctagtatgcctgtggaatctggtcctatcagcccaagtctgttaaatcaaataaaaccagggcttggtgcttcaccttgtcttctaccataccactgggtttcctgtggaccatgcagataacgatgatgggctcagttggcttgatagtgataactcctaaagcagctgcttctaagtgtggttctcaatctgaggaagttaaaaaaaattttagtgactagaacccacttctcagctactcttactaaataaatctgtaggggagtgatagttttcaatttttttttttttttttttttttttgagatggagtctcactctgttgcccaggctggagtgcaatggtgcgatctcagctccctgcaacctctgcctcccagcctcaagcgattcttctgcctcaacctcctgagtagctgggattacaggtgcgtgccaccatgcctggctaatttttgtatttttagtagagatggggtttcaccgtgttggtcaggctggtctcgagctcctaaccttgtgatctgcctgcctcagcctcccaaagtgctgggattacaggcatgagccactgcgcccggccttcacatattttttgaaataataggccagttgcggtggttcgtgcctgtaatctcagcactttgggaggccgaggtgggtgggtcacttgaggccaagaattcaaggccactctggccaacgtggtgaaaccctgtttctactgaaaatacaaaaaattagccgggtatgatggcatgtgcttgtggtcccagctactctggaagctgaagcgtgagaatcgctttaacttaggaggcagaggttgcagtgaggcgagattgcgcccctgcgctccagcctgggtgacagagcaagactccatctcatttaaaaaaaaaaaaaaaaaatcataggcagtaaaggttgagaactgctgcccaaaggacctattaaactatagattcccaaacctggccaattatcaaaatccctcaagaaggggcaatggggtctaagggaccccactggaagagattagtagtccaagagtgagatgaactgttggaagtcctcagacttccaaactattagaatagttttgcttcctcaaaatagagtagttttgcttcctcactaatttttctgtattgattagaaccgtaacaagtgaattaaacaactacaaaatagttatgtgggcaacagacattattgtaatgaagtgaagtttggctcaggccttggaacacaattgcgttttggattaaaagtaaaaatatttattaaatcatgtgagattatgtgtaagttttaaaaaattggtcttatacaaaagtgttgggggtttttttttttttttttggctgaatttgtttttaagcaagacagaatatttatattgttggagagtcacagaggaggtgtgtttgtggatttaaatgtggagacagtgtgccttgagtgccctttatcagtctgattcgagccactgataatcatggatttgaactaccgccccccaccccctgcgcctagatagggtctccctctgttgcctaggctgcggtgcagtggtgggatctcggctcgctgcaacctctggcttagcctcttggagtagctgggactacaggcacacaacaccatacccagctattgttttttttgtttgttttctttgttttttttttttttttttttttatagagacagggtttcaccatgttgctcaggctggtcttgaactcctgagctcaagcaacccacctgtctcggccttccagagtgctgggattacaaggcctgagctaccatgcccagctttcacttcatttaaaaaccatagtttttaaaatccacagtcctactgacgaacacagaggttgtttccagtgttttcatttgcagtattgcagtgattgtttttgcacatgcctccttatgcacatgtgctgtggcttctgggacagagaatggacatattaagtgttttattgatcctgctaaattgtccttcagccaaactgctgcagaggtgatagagatgaggtgggtactcaggagaattgtgcccagtgcttgtgtgctggttgctgacctggaaacattttattaaaaatgctcgattaggttagtgatgtaaagatgattaccaggtgatagtaaccagaataattgtgtcaaaacatcaagaattatcaagagatgccaggcatggtggctcacgcctgtaatcgcagcactttaggaggccaaggtgggcagattgcttgagctcaggagttcaagaccagcctgggcaacatggtgaaaccctgtctctaccaaaaatacaaaaatttgctggatgcagtggtatgtgcatgtggtctcagctattcagtaggctgagacgggatgatcacttgagcctggaaggcagaggttgcagtgagccgagacaatactgcagcccgggcaacagagtgaaaccctgccttaaaaaaaaaagagaaaaagaatcatcaagagaaaacttaatttgatgtgttctgtgttttctttggtgcttcctgtcagtgttagaaactgtaggttatatatttaataatttttctcctgtatttgttcaacttgactataaaatattaactccaaatgcctagaatttcaaaacacctcttcattataaagtatcagctatttctgagtccccttaagctcatagtattgtgtcattgtaaaagatctgttgtgaaaaataatttttgtcaacatgaaaggtcttaatgtgtctcccagtttacattttacatggtcttttccgtgtatttttatagttgacatatatagctttttttgtaaatatactttcctatatgaacatgccaaggtttacttaagcattctcttattcttggacatctaaattgcttcttattttctattgtaaataaagtgctagaagcttccttcctgaaaagttatttactgttcagctagtatttccatctgtgttcctcaaaataagattactgagattcatgtatgttgccagaaagattgtgaggtttaacagtgaatgaggaaaacttttaacacttaaggtctctcaagtacagcaagttttatcattttactttttttttgagataaggtcttgctaagttgcctaggccagtctcaaactgctgggctcaagcaatccttctgcttcagactccggagtagctgggattataggtgtgcagcaccacacccggctttcttgtattattattattattatttatttatttatttttgaggtggagtttcactcttgtcacccaggctgtagtgcaatgacccgatgtcggctcactgcagcctccatctcccaggttcaaatgattctcctgcctcagcctcccaagtagctgggattataggcatgcgccaccacacccacctaatttttctatttttagtagagacagggttcctccatgttggtcaggctggtctcgaactcccgacctcaagtgatatacccgcctgagcctcccaaagtgctgggattacaggtgtgtgccaccgcgcctgtccgctttcttgtattatttttatcttgctgtagtggttgtaggatttttgcctggtgtgttcattggagtgtgtgtgtgtgtgtgtgtgtgtgtgtgtgtgtgtgtgtgtgttttgagatgtatgactgtgttggttgtgttgtcatgctttgaaggttcattcatgtgctcatctatttttctcattatacttagtcacttaaaaacctcttttatttggatttctaatttttacagttcctgttttattaatcaccttcttatctgaaagtgaagtattgaattagaaatttgtgtgtccctgatcagaagatcacgtacacaggaacaagataagttgagggatttagcagttccagaaatgggcatttgtgtcttccagtggagaagcatctgcaaaagaattgtgcagatttggccgggcacggtagctcatgcctgtaatcccagcactttgggaggccgaggcgggcagatcacctgacgtcaggagtttgagaccagcctggccaacatggtgaaaccccgtctccactaaaaatacaaaattagccaggcgtggtggcgggcgcctgtaatcccagctgcttgggaggatgaggcaggaaaatcacttgaacccatgatgcggtggttgcagtgagccaagatcgcaccattgcacaccattgcactccagcctgggcaacagagtgagactccatctcaaaagaaaaaaaaaaaaaatcatgcagatctatttaactttccataaagtgagacagaactccgtaagttagggtaaatttaaggacatgtaatgtgctcattatgttattaggaaattaatggtgaaaagggtgttttggcctcgtgtggcttcttgaagatgacctgtgaaaacagcaaggcctggaagggaactgaattgggcagatgcagatttgtggactctagtgaactatggagaaacaattaggactccaggatgagatcttcttaggttggtgcagaagtaattacagttttggactgtgaattttaaatcattataactaggcttaaacacatctttattaatcaaaataggaaccattacagtcaacacattttcaccaacaagaaataagtttgtttcttcctgcagcacaaaaatccgtgcttcaggattcaacaaactgttggaaagcattttctgcatcctgctggttgtggaaatgttttccctgcaaaaagttgttgagatgcttcaagaagtggtagttggttggcaagaggtcagatgagtatggtgaatgaagcaaaattttgtagcccaattcattcaacttttgaaacgttgtgtgacgtgaggccaaacgttgtcatggagaagaattgggccctttctgttgaccaatgccggctgcaggtgtagcagtttccggagcatctcattgatttgctaagcatacttctcagaggtaatggttttgccaggattcagaaagccgtactggatcagaccagcagcagaccagcagcagaccaccaaacagtgaccatgaccttttttttttttggtgcaaatttggctttgggaagtgttttagagctgcttcttggtccagccactgaactggtcatcgccagttgttgtataaaatctactttttgtcgcacatcacaatccaattgagaaatggttcgttgttgtgtagaataagagaagacaacacttcaaagtgatgatttttttttttaaatttttgctcagctcctgaggcacctacttttcaagctttaaataagtagtgaccctgggattcctagatcccttgtgggtcctgatgtaatgtggtctgcagatgttttcagtattcataaaggtgaacaatttgcctgttaactaaaatatgaagttgtgttttgggtttgtgtgggtcatccattatcataacgtctgagtcttctttttattgacttagagtcttccagtcctcttattttgcttggtggtagcatgtacaagatagaagtttaggcacaataatctattcagtggctgtaatagcagcttgtcctgcctggttttttttgtttttgtttttttgtaatagcattgaggagatataattcacccatttaaagcaggtgaattatatattttatgttttaatgtattcctagagttgtgcaaccatcaccagagtaagttatagaacattttcatcaacccaaaaagaaactttgttttcattaatcgttcctcctcatttctcccctaaactcccagcactaggcaaccatcagaatactttttgtctccacagatttgactgttttggacatttcatattaatggaatgtacgatacagtagtataatacattaattttggaaggccaaagttaatggccttcgatgtctcccttttttcacttagagtaatgtcttccaggtccatctgtgttgtagcctgtatcattactttatcctttatgtggctgagtaatattccattgtatggttgtaccatgttttgtttattcatcagctgatggacatttaggttgttttccattgactattaagaataatgctgccatcatgcttgagtttttttttttttattttaggggtgggtggtgggcagagggcacagtctcgctctgttacgcaggctggcgtgcagtggtgcgatctcggctcaccgcagcctcctctaccccctgggttctagtgattcttgtgcctcaggctcctgagtattatgctgcagtttttatgcgagcatacattttcagttcttttgggtcaatatctaggagtatccccacttttggatctgagtgttaattgagcctgttgtgttctcacattccctgtacttacatggaaatagtgctttgcctaaaaagaaaaacaaaagacctaattgcgcaggcgagtgagagagaaagagagagagagagaaagagagagagagagagagagagagagagagagagagacacgctagggttatttccaccctgatactctttgtgtagtcttggtatgactagcaaagaagcaagctccaagttgtaatttgcttccaagtttctggcttctgtgggaatttctgcatcttagtaatgacaattttcagttacttgcagtaagtaaattatcagccagtcttactctgtattactagtctaggaggagtttacttgtatattgagagaaatgctgcagcttttaccttacttctaatggggattaatgcttacttaacttaccgtttcggaggcaaataaaaagtgtaggaccaattatggtcttgaagtgagagagaagtctggctagtgaatggtgattggcaactccagttgactgttcatggcatcttagatctgtgaggagagaggagggaaggaaagttcaagctggtctttatggtaagttctggaacatttccctgtgtcagtgggtcatctgttcattcactgtgtaaaatggctgaggaaagttttcattttcatacttcttcattgtgtaaacctttgatttttagtgatttcagagtttgtttttataattatttaaacatgtgaagaggatacagagtaacatatcgaacctctggttatcttccactgggatttgacatatttgagttccttttcttccttcttccttccctccctccctctctcttttcttaaggaatgcaactactcagattcacctgcataccgttggcatacctctcacttcctcagaggtgaccagtcctcagagcaaatgtgtgccctttccatccgcgcttttatgctctcatctatgtttacatctattagtacactattgtctatatttttaaacattacataaatggtgtaatttttacttttaattctgtagtggtatttctcaaattaagttctgcataaaacatttttatagatatccagtgttagcttaacgtttttcttattgtggtaaaatatacataagataaaatttaccatttttgccatttttaagtatacaagtcagcagcattaagtacattcacagtgtagtataaccatcaccattgtccattgctggaacttttcctcatcctaaacagaaactctatactcattaaacaatagtttatcactccttccctgcaactagatgctggcagtcaccattctactttctgtctctatcaatttgcctgttccatctaagtggaatcctacatatttgtcgctttgtttctggctttttttttcttcttgtgatgctttgttttaattatgtttctggctttcttcatctagcaggctgattccaaggttcgtccatgtggtagcttgtatcactttaattcttttagagataattaatattatgttgtctgtatatgccacattttgttttttcattcaaccttgatggacatttggattgtttcccccctttaactattgtgaataatactgctatgaacattgatggccaaatatttgtttgaatctctgatttcagttcttttggttctatacctaggagtggaattgctggatcatatgataattctatgtttaactctttgagggatggccagacttttccaccatagctaaatcattttaccttcccacaagcaaagttcaagggctctagtctctccccatcatgtcctttgcacttttttttttttttttttgaggcagagtctcactctgttgcccaggctggagagtagtggcacgatctttcctgacctcaagtggcccctgctttggcctcccaaagtgctaggattataggtgtgggccactgcactctgccaatttttaaattttaattttcatttatttttcgttttttcatttttagttttttttattttttgaagggataaggtctcattttgttgcccaggctggtcttgaactcctgacttcaagcaatcctcctacctcggcctctcagagtgctgagattgtaggtgtaagcccctgcacctggcctttactctcttgatagtgtcctttgatgcacaaaagctttcaattttgatgaagtctattttttctcttgttacctgtacatttggtgtcatatatctaagagaccattgccaaatgcaatgtcgtgaagctttccctcagtgttttctttccatagttttatgattttagctcctaagtttaggtctttgatccattttgagttaatttttgtatacagtttaagagtcagacttgagactctgggcgcagtggctcacttctgtaatcccagtactttgggaggccgaggtgggtggatcacctgaggtcaggagtttgagaccagcctggccaacatgccaaaaccccgtctctagtaaaaatacaataattagccaggcatggtgatgcatacctgtggtcccagctactcgggaggctgggcaggagaatcgcttgaacccgggaggtggtggttgcagtgagctgagattgtgccactgcactctagcctgggcgacaaaagcgagtctccatctggggaaaaaacaaaaaaaaagtcaaacttgagtcttttgcctatggatatccagttttcccatcactatttgttgaaaagactatcctttctcaactgtgaatggtcttggtatcctagctgaagttatttttattactatgttacttaggaatgcacataaggccgggcacagtggctcatgcttgtaattgcagcactttaggaggtcaaggtgggaggattccctgagccgaggagtttgagaccagcctgggcaatatagcagcaagaccccatctctatattttaaaaaaagaagaagaaaaaaaaaacccactctgacacataattatttaaacttgtatgcattcttttctttctttatttttaaaaaattgagatagcagcttactctgttgcccaggctggtctcgaactcctgggctcaagcagttcctcttaccttggcctgaagtgctgagatgacaggtgtgagccactacatctggcctgcttacagattataaaaagaaaataagtttacaaattaaagacagataaaatgacagaatcagtaaaattaaaatttcttttatggagctgatgatgtttatcccaattggtcctctcattgtgaatatggtattgttgctgtggcagatttggaggctttggcaatggcttctattaccttgccatgaggtaactcagttccctcattacttttctctgagaactgtaaaaccttggaggggtgccctctgcccttcgcttggcatgtgtattatgcggggatcaggtcttactctgttcttgattgttagtacaaacgagttaaaatcctgttgtttggccttagcctgatggtaaacacaacagcacacatgggctgtgaaatccctgggcagctctgtgtttctagggaagcatctcgatgatccagaacaggcttatactgatgttttagtgtaattttgaaatgaaaacactgcatttaaaaaattctcatagagaatgtatagacctggagaagtgttagcagacccagtttaagacatgtctcaatattacggaacattgctttattccctgtcctgcttgtacatttaattttttcacccacttttaaacaacttgggtactgtggcctgtgcctgtattcccagctactagggaggctgaggcaggaagattgcttgagcacaggacttcgagggctatagtgagctgtgtttgtgcctgtgaatagccattgtgctccagcctgggcaacatagcaagaccctgataccctgggtttttaaaaaacaaaacaagatacatgctgacatttctagtttggcaggcagagcttgttctgctccccaccctcccttttcctatagtaaccatttataggacatctcactgttgtctactctgtgttgcctctgcttccctacgtggtagatctaggaatcttaggatttcttagttttagctggtgatccatatatttttcttaattccattgtaacttcagcttttcttattgcttgtaagaaggctgtttccattgaatacaaacaaaataaaagcttttatttgtaatcttagagataggatgtttgtatttaaaaataattgtgctgtcaaaattctgtcaagttggcttctaccatattagtttttttttttttttatgtgatttatatgaccctggagtaccttgtcttctcactgttaaattctcaactgagttgtccctatttaaagtgtgagactgtgccagtttgattttaaaatattgcaagtgcgttatggcaagataaagctgcaaagaaagaaccttcatgttcctttgattataaatgcttttggcacttgtttctactggaagataactttttcctgatgtgtttttgaggaaagaacctccaacgctctagacaggtctgggggcaaatgactaaaacatcaactgaggccctgggctgtctccatgaggatatcccctctattgtctctgaaatgtcccagcatgtggtgcatttcttgttagtgtggactcctctgtatataacacccattatttatgttctgtgcataacatgaaatagtgccctaatgcaattccaggatgtaattcaacatttctataaaaatacaatgtttttgtacatttgcatcaaacaataaccagataattatatttgttaagaaaatagtatttttggctgggtttgatagctcacgtaatcccagcactttgggaggctgaggcgggtggatcacttgaggtcaggagttcaagaccagcctggccaacatggtgaaaccccatctctactaaaaatgcaaaattagccaggcatagtggtgcatgcctgtaatcctggctgctcaggagactgaggcaggagaattgcttgaactcaggaaggggagattgcagtgagctgagattgtgccactgctctgtagcctaggcaacagagtgagactctgtttcaaaaaaaagaaaaaagaaagaaagaaagaaaatagtatttttggtatttgttttcacaaactagagcatttatgtgaaataacattgctagtattgatattataccatagtataatacttagttcttcaaatgatgtatctctgctgatcagctacatgatatctacgtgagttgttgcgtgttttttctctttttttaaagagcagtgcatttttgaatgcttttgaaaaattgcagtaaaatacataaacgtaacatttaccttgtaaccattttaattggtacaattcagtgacattaagtacagtcccagtgttgtgcaaccactgttactgtctagtttcagaatgttttagctccaaatgggtaccctgtacctgttgaacattcagtcctagtggcccaataatcttctttatgtctgtatagatttgcctgttctgcatgttttatataaatggaatcatgtcttttttgtctggcttcttttacttagcatagtattttcaaggttcatctacgttgcagcatgtttcaatacttcgttcctttttatgtccattatgaatataccacatttcgttaatgacagttctttggggtagctacattttaaaacattatagtaaaatacacatggcatacgatttactatcttaaccacgtaagcctgcagttcagtggcattaaatacattcacattattgtacaattatcaccaccatctgtttccagaaatttttcatctcctccaattgaaactctgtatacattaaacatgaactctccattctccccttcctccagcccttagcagccaccattctacatttctatctttctgacagattttactactctaggtacctgacataagttaaatcaaccagtatttatccttttgtgaccagcttatttcattagcttaatgtcctcaagattcatccatgttgtagcatatatcagaattactttccttttaaggttgaataatattccattgtatgtatatgtcacaattcgtttctccatttatccatcactggacatttgggttgcttccacgtatcgcctatcttgagtcatgttgctatagctgtacgagtatctatttgagtccccgctatcaattctttgagtatatgcccagaagtggaattgctggatcatatggtaattccgtgtctggtgtttttgaggaactgccatgctgttttccacacagctgtatcattatatgttccctccggcaatgtatgagggctctaagttcttcacatctttgctaacacttagtatttttttttttatggaatagccatcctaatggctactttaaaaatatatttaactttatttatttatttttaaatttttttatagagatgccatcttactatgttgcccaggctggtcatgaactcctgggctcaagcaatcctcctgcctcagcctcccaaagtgttcagattacaggaatgagccaccatgcccagcccaaatttaaatatgtaactaaacacatagcagctaacaccaagcctttaaaaatatcattaataggctgggtgcagtggctcatgcctgtaatcccagcactttgggaggccgaggtgggcagatcacctgaggttgggagttcaagaccagcctgaccaacatggagaaaccctgtctctactactaaaagtacaaaattagctgggcatggtggcacatgcctgtaatcccagctactagggaggctgaggcaggagaattgcttgaacctgggaggcagaggttgtggtgagccgagattgcgccattgcactctagcctgggcaacaagagcaaaactccatctcgcaaaataaatatctgtatctatatctatatatcgaacttcttaccccagtgatccacccacctcggcctcccaaagcactggaattacaggcatgagccaccacacccggcctattaattatatatgcacctataatcccaactactcgggaagctgaggctggagagtggcttgaacctgggaggtggaggctacagtgagctgagctcacactccagcctgggtgacagagcaagactctgtctcagaaaaaaaagtcattaataaatgtgatctttttttttcctgtacaagttgtcaaggaagtatgaccttcttaattgaccctttgacatgaactgggatgagatcgtggaggatgttgaggagacagttgttaccatagtgcgcttctaaaaactaattctatagatttctttacaaaaatttgtttaaattattatgagtacacaataggtgcatctatagatttcattaccctcaaataaatgtacaaggcaatgcagagaaatacatagtgtaacttggtagacttgacctatcaagttactcttgaatatgttatgaagcctgtgtattactgggggcagcaaacttctcctgttatgatagttgttttcagctttgttgaatctgtggtctctgtcttaactacaactgagaaagcagccatcgacaatatgtagatgaatgtacatgactctattgtaataaaactgtgtacactgtaatttgaattgcacataattttcatgtgtcccctgtataattcttcttttgacttcttttcaaccattaaaaaatgtaaaaacaggcatggtggctcatgcttgtaatcccagcactttgggaggctgggtggattgcttgagcccaggagcttgagatcagctttagcaatgtgacgaaaccctgtctctacaaaaaattagctgggcatggtggcatgtgcctgtggtcgcacctactcgggaggctgaggtgggaggattgcctgagcccgagaaagtcaaggctacagtgatttgtgccactgcactctagcctaggtgacagagtgagaccctgtctcagtgaatgaatgaatacattattagcttgtggactatacaaaaattagaggctggaggtgagctgggtatggccattgggcatggtttactgacctctggtagagaggaattagtatatgttaaaggtggtggaactgtctgatacttgcgttcttttagaaatactttggagttagctttttggttcaggcaaaggaccaaagagttaggagagtcaggctggtaaagaggagtggtgggcccatagcaacaggtcctggagtctttaagaggaggtgtgtctcatgtgacattggttggttggacaaaagcctggcatgttgtcaccttccataataagtgtttgtgggaatgtaggtaatgagaaggaggagtaaagggttcctgaaggatgaggaggagggctggtggctgccataggaagtgatcaccattttggcggacctgtcttagagtaatgaccatcatactctctcactccttgtgaactcatgaagtcccatggctgctaaagctaaaggtcaagtggggacttctctgggcactgggcttggcaccccacagagctgtggagtgggcattaatgtccctgttatatagatgcagagactgagaatgaggactgttggtaacttttgagagggcactcagctagaaaagtctgagccaggatgtcaagtcccatggctttacctctgtggtcctaatggttggtgtgttcaagtgagatccgttttttcatatttggttttgattattgattttcatgcattttttttcttttttgagttagtatattctctctcttttctttttttcttttctttctttctttctttctttctttctttttctttctttctttctttctttctttctttctttctttctttctttctttctttcttttctttcttttctttcttttctttctttcttttttttttttttttttgtgagacagaatctcgctctgtcacccagactggagtgtgcagtggtacaatctcagttcactgcaacctccacctctcgggttcaagtgattctcccatctcagcctccgaagttgctgagattacaggcacctgccatcatgcctggctagtttttatatttttgtagagacagggtttcaccgcgttggccaggctggtcttgaactcctgacctcaggtgaaccacctgcctctgcctcccaaagtgctgtgatttataggcatgagccactgtgcctagccagtatttttttctttctttctttttctttttttcaggttcattgaatttgctttgagacaggatcttgctctgttgcgcaggctgaagcacagtggtgcaatcatggctcactggagcctcaatttcctgggctcaagcaatccttgcacctcagcacccctccacccccacctactcctttcccccaccagtagctggaactacaggcgccagctaccgtgcttggctaattttttaaatgtttttatagactgggtttccctatgctgcccaggcttattaattgattttctgtgtgatcttagggaaatcgattatttcccataaacattttttaaattagaagttaaattctgcctagtttgcctcacatgattattgtggatgactgaatcagagaagaggtaagagctctttgaaaaatatgaagtaccatacagaagttagatgctttgtcctggtgagacccctccaaagcacagctaaggaagtgtggaaggcactcttatcacatcatatagctttgaaagcctagcattgaaagtatgaacttgattcttttggagaaatcctttggctctcagtgagtttactttctattaatgactatattaagcggaatggaaactgaaagaggaaagaggaggaagtcagaattaaataggaagagtaagcccatagcagagtccagatttagaccccaagctacttggaatgatactggacaattatgggtgtgtttaatgattgccctgagtcatgaaaacaaaaggaggctttaaattatgtctggcttagtaatatagcatattttatcattattcaagttttagcatgtaaagaggaaaagtgtgcagtacttacacataccattttctattagcgaaactaaatgaggccgctttaaaattatcagtgttcacagtatcttccaaaagacatgtaaatgtataaatgtataaaaaatatacatataaattttacagtttggtgagctatatagtagatctcttattttgtccataggtcgtaaagatcttatactgtatttaggaacaaatataacttaagtgggagtcctttacagggctaataagtaagcattattttgataaagtgctgtgttgtctacactaggtatagtagaaatactcttggaatagtaatcatcccaggccctactttggagtggaagaaatagtcaatgtagaactttatagtacattgtacgtagatgtgcctgctaataacttctgtagacagcaaagtttaagagaaattaggtggtaaatacaacatatgtatctaaataaatttggtctgagagatttgataagatgaaacagtacatagtccagaaaatttttatactcaaagaattgtagaaaatatcttaaatgttttcagttttgtgtatatccagagaatatcatcctgtaatctgctggttggcaacccaatggcagtattagatgtatgtttttattttgtttcgtttgctatttgtttggttaagagagttacctaattaggagtgtggaaaaaaaaaaaagatttattatagtagtgggcttttgtttgacttcagacatttttgttgttacaacaatattagtgtcttgtttgtgaaatttgtttaccgggaagccaaatacttagaataacttttagtttatcattatcatcatcatcatcatcatcatctccatcatgaaaggaagaagctaccaatgttgctttattctgcaaacaatataatagatgcttgttgaaagtatggagtgaaatcttaaatatatctgttaaaaagagtacaactggccaggtgtggtgcctcatgcctgtaatccaagcactttgggaggccaaggcgggcgaattgcttgagccaggagtttgagaccagcctgggcaacatagtgaaaccctgtctctacaaaaaaaatagacaaaaattagctgggtgtgatggcatacacctgtagtcccagctacagtggggctgaggcagggggtattgcatgagcccaggaagtaaaggctgcagtgagctatggtcgtgccactgcactccagtgtgggtaacagaatgagaccctgtctcaaaaaaaaaaaaaaaatagtacaactttaagcaggatgtgggtacatgactgtagtctcagctacttgggaggctaaggcaagagggttacttgagcccagaagcttgatgctgcagtgagatgtgattgtgccactgccctccagcctggggaccatagaaagatcccatctcttaaagaaaaaaaaaacagagtacaacaactttggtaaacttggaatataaagatatttccttaacctattaaagagctgataaagagtggtactttcaaaccagtacacattatgtgaaacactagaggcacttcccatttgttaaaagaaaaaccttagccaaattaaatttaagttttgttttttttttttttgtttttttgtttttttgtttcttgagacagagtcttgctttgtcacccaggctggagtgcagtggtgcaagcacagctcactgcaacctccgccttctgggttcaagtgattttcttgcctcagcctcctgagtagttgggactacaggtgtgcaccaccacgcctggctaatttttgtattttttgtagacacgtggttttgccatgttgcccaggctggtcttgaactcctgggctcaaggaatctgcctgcctgggcctctgaaagtgctgggattacagatgtgagccactatgccatttaacggtttaattgagcaaagaatgatttgcaaattgggcagcctcccgagccagagtaggttcagagagactccagcacagccatgtggtggaagaagatttatggatggaaaaaggaaagtgatgtatagaaaagagaagtgaggtacagaaacagccggattggttacagctcagaatttgcctttttagaacacaagtagaggtttgaacagttggccacctttgattggccaaaacccggtgattggcacaagagcaggttccagtctctttacatctccatttaggttatagttcactatggatggaaaaacctgtagatcaaacttaaaatacgtaaggagacagttttaggctaaacttgatttaacacattaaatccataacaagacaggatgcctgccctcaccatgttatttgatcttattttagtaattctagccaatgtagtagggcaagaaaactgcctgcttggttacaaaataaacatgcaaaagtcaatatttgtaatatgtgacagaaaatataatttaaaaaagaagaagccgggcacggtggcccgtggctgtaatcctagcactttgggaggccaacacaggtggatctcttgaactcaggagttcaagaccatcctaggcaacatggaaaaccccgtctccacaaaaaaattagccaggtgcggtggtgcgcgcctgtagttccagctgctttggaggctgaggttggaggattgtttgagcctaggaagcacaggttgtagtgagctgagatcatgccactgcactccagcctgggtgacaaagtaagactctatctaaaaaaaaaaaaaagaaagaaagaaaaggaaagaaaaagaaaaaagaatttcacttactagagcatcaaatccctaagataatttagaacaaagccagaaaagtgaagaaaatacaaaaatctttacagtgagttgcataaaaaaaaacaacttgaagatgtcagatcaactcattaatgtgttgattaatgtaattaaaatcccactgttttttttgtggggagggtgggagagtcacttgttaaaatgattctaaagagcatctggaagaataaacaggcaagaatagccaagaacattttgaaaactaaagatgagtttggaagacgattggttttgtactatcaactacttatagattttacatgaattttaaagggtaatctgagtcctcgaatagacagaaatagccatagatctgaaagaacacttaagacctgatctagctatccgtgagagtatgtataatcaaagtctttgtgtgtgaatcaggagtctttcaggtgcaaggtaaataagctcataattgcttaagcaaaggtggtatttgctttagtgactccagagaaagctcaagtgcctgtctccccctgactttgattctttttggggttggctccattctctcctgttgctaatggcttcctttgtgcagccagaggaaaggaggtgtggttttttgatacttccagtcttctatttttatagcttgagatcaaagagggaagtgaccttccttagagtcagtgtgtaaagtcctaaggaagataccacgtggggtgctggggccatgtgcccatccctggcccatgacgatggggatgctacactaactgggggccacccatggctgttcctgccttgaactgccaactggctttgcagtgcagcttcaccagaatcacatggaatagtagggataggaattgtttcccaaagagagtgtgtggggtggtaaaattactagtgggggagtaaggggacaggccattgggcagactggagcagcatttacttactcagtcattgagaaaaggatggaacattcaataaagggtgctggacacagtttgtgctctaaaaattttgtgtttcacctattaatttatccctccccttagcccctggcaaacactggtctgtttactgtctccatagttttgcctttcccagaacgtcatgcccttggaatcatacagcaggtaaccttttccagttggcttcttttatctagtaatgtgcatttaagattccttcatgtcttttcctggattgataacccatttctttttagtcctgaataatattccattgtatggttgtaccacagttgatccattcacctactgaaggtcattttggctgcttccaagttttgataatttgaaaaaaaaattttgagacagggtgtgattgtgtttaagatactggtctcctgaacaactgagctcacgtgaacccctctcctcagcctcctgggtaactgggattacagctatacaccaccgtgcccagtgtgacaattatgaataaagctgctataaacttctatgtaggtttttttgtgtttggacattggttttcagttcattatggtgaataccaaggagtgcaattgctggattatatggtaaaagtatgtttagtttgctaagaaactgccagctgggtgtggtggctcacgcctgtaatcctagcataatgggaggctgagacaggaggatcccttgaagccaggagtttgagactagcctgggcaacatagtgagacctcatctctacagaaaatttaaaaattagctggtcgtggtcttatgtgtctatagtcctaactgcttgggaaactgaggtgggaggatcacttgagcccaggagctggaggtggcagtaaactgtgatcataccactgcactgcagcctgggtgacaaagcaagaccctgactttaaaaaaaaaaaaaaaaaaaaatgagtcagagggtaaggaagcaaaaataagtaaataaataaatagaagataaaagaaaaatctatctttcaaagtggccgtgccattttgcattcctaccagcaatgaatgagagtctctgttgttgcacatcctcaccagcatttggtggtgtcagtgttctggattttgaatattctattaagtatataatgctgtctcacttgttttaatttccaattctttagtgatgtatgtcattaagcgtctttttaatatgtttacttatcatatatgtatcttctttagtgaggcctttgtttaggtcttctgcccatttttaaaaatgggtttattttcttattgttgaatagtatgagttctttgtctattttgaatacttgtcttttgctttatttttgtgttttttttttatttttatctttttgagacaagttctcactctgttgcccaggctggagtgcagtggcatgaacatggctcactgcagcctcaacttcttccaggctcaagcaatcctcttgcctcagccttccgagtagctgggaccataggcgcacaccaccacaccctgctaatttgaaaaaattttttgcagaggcggggtctcaccatattacccaggctggtcttgaactcctggcctcagtcaatcttccagccctcagcctcccaaagtgctgattataggcctgagccacttagcctagcccagaatttatttttttatttcttagttttgaaaaaatataggacctcataaaagtcagtctagatttgtacacattatgtttttggtgtatatgtaaatggattctttgtgaatcaatttggttttgtttttttgcttttaaaaataccagcactgggctgggtgctgttgctcatacctgtaatcccagcactttgggaggctgaggcaggtggatcacctgaggtcaggagtttgagatcagcctggccgacatggtgaaacgctgtctctactaaaaatacaaaaattagctgggcgtggtggcacatgtcagtaatcccagctactcaggaggctgaggcaggagaattgattgaacttgggaggcggaggttgcagtgctccaagattgcgccactgcactccagccttggcgatagagcaagactttgtctcaaaaaaaaaaaaaaagaaaaaaaccgcagcagtggctggccaaggtggctcacacctgtaatcccagcactttgggaggccaaggcaggtagatctcttgcggtcaggaattcaagaccagcctagcgaacatggcgaaaccccatctctaccaaaaatactaaaattagccagatgtggtagtgcacacctgtaattccagctgcctgggagactgaggcatgagaatcacttgaaccctggaggcagaggttggagtgagccactgtattccagcctgggtgacagagggagactctatttaaaaaaaaaagaaaaaaaaggctggatacagtggtgcacgcctgtaaccccagcactttgggaggctgaggtgctcagattgcttgagctcaggagtttgagaccagcctggacaacatagtgagacatcatctcttaaaaaaaaaaaaaaatacctgcacttggtcaaaagatttcaacagtgcagaaaaagaaagttgctatatcttttctccaaattagtcttgtttctagtttcattatccaaataatcactactaatagttaaaacattttaaatacacattgggagtttgtcctatttaatataaattatttattgagcaaataatcactgctagtatattttggatactggaattttcatatgtaggggtccttgaatgtaaggtgcccctttggtagttctgtgcttcttttacctgtactgtaacatagggaaagatgttacaaatggttagtatctattcttaaacaccagcccttccactaaaggtaaacaacaaataaatatataaatgaagttttggtattgggattacacgggttaaacacatccatatttcattattaatatttaagaatataacaaacttcttattggcatttggaccttgtagctagggaaagattaagctttgtttatttgtgctttgttttttttcttcactcagatatttgagggtttcccatttgaggaatacatttattaatcaagctttagttgcaagatatttgatcttagagaataccatcaaccattcttctttaagcttcctaactttacccaaatgtggttggatctactcaagagtagtttgggtagttcagaaattttattggaaggggaaataatttttgacccaaatttgataaagcaactcttgagtaatgatttcttttcttgttctctctttataatcagttgaaagtagtagtaaggctgggtggcaaaagaaagaggcctggggagaatcgggtggttttcattatctcttttcatagcagctaagtgggaagggaccaagaggaaatcaactgaaaaaccatccttctgaaacattggcctaaaaaagtgtagtccagaaattgagtgcaactggcagtggcatttaaaaggaatgctctaatttctaggaaagcaggcacgagtacctcttaaaagaagaaaaaaatgaaaactgtaatttaggacacacagacgagtatccattccctgtacttttttactctcgtgtcctaaccaaggaagggttaccatagcaaatatggcattccttagccatgattcactgttgtaaatgcctgcagcattcataaaagtaagatatatgggctctttctttttccttttgaatccgtatttctgtatttaaatctgtatgtcaacatctgtattttctgtctctcttgtttttttaaatcttgggagatggtacaaattatttaggggagtgaataagtttcttgtctacaaatagaggagagagaaggctttttgtctttctgctttggaactggagagcttcctatttaggcacggcctttttcaagtgaccttgtattgttatcagtactgtagaaggtaggcacattgtacagactttaaaatgtaaagcttttaggcattccacttgtaaaccttggctttttaaagaaaattacatgttcattgtgaatattttcttatcgccctatctctgtgcacatgcagacttcctttggctacattctgaaaggtgtaattgtcttctttaaggacagtggacatctatagttcttaggtcaaattgtcctccttctggttttgtcagttctcagccacactgtgtgagcatccattttcttggatcctggtttggagctcattttaaggaacatcacgtcccttttgagactatgtggacatcagggtgggtagatgttcccctgtgaacagaaggttcctccctaaggaggtgcttctctgtgttgagtcttgcatctgggcacacagagcccaaagcaggaagagttgagtctgaatagggaggcctgtaagcctcactgctctgccacggctaggcctggttggccatgctctaggagccctcaggaggctctgaagtgattctgcctctgggaattttacagaggagctaatatttgagatctccaaagctgagtgagagggtgattcttgaaaaggcaaggcatgcctcggtccagttctgtggggctgtcatagaagagggctcggaagctcttgtaagtgaggctggaaaggtaggcagtgttaccataaggaggatggaaatgacacagataggctaaggaaggggacctctgtaatcatgcagcgatggcctagaaaagggacaggctcatggcagggagaccagtttggaggctgccacagtgttctactgagagagaaaaagaattaaactcaaagcacggtcagggaaggtggaggggcaggatctgatttggaagtgttttgttttttggtttcgggtacaaggatttggaaacctcttgactccgtagatgaaagaatatgaaccaaggaagactgaagtttccactcagggaagaaaacacaggagtaggaatcgatttaagagaatggcaggcagttgtgttttgaacttgtttagtgtgaggtgacatctttgtagggatgtctagctggtagctgcaagtacaggaggctagatgtgtgcatgagtttcttggtaaagatccatctgcagtctcctgaagatgtcactcagattgcactgccatcaccccagggcccaagagaagacactcgcttctcttggtgtctaggcgggtttgaaaaaaatcaaacagtacaaaatcatgcaaattcagacctctactccaccccagaggaaagtcctgctagcagtttttgtgtattcttccagaaatctgccttgcaacatgtttgagtatataaataatcccagaaaggatgggccagacactagcagaaactcaccacacacactgcgctgggcatgcagaaacttcgcgaaatatactggtgtgtgctgtcactgcattcctggatttgggggtcttctgtgtccccaggtatcagtattgcccacaggctgaccactgagctccttccacagccgactcacgtcaccctctttcgaagtgtgtctgcaaagctagattattagattgtttctgctttgtgttttctttaatgaattttttaatgatgaagttcttgttttaaaaatatacagtggtaattaacatgtatgcatttttcttaaaatgacccccccagtccctcttcagatgtaatcactgttaacagtatcgtatatagaccctgttctgtgtggggtgggcagagggccggttagcgggtggaacatgcatactcacaaccatatttttcacatgggaaaatataaaggtgacaacaaatctcctggtctgtaatctcatcaagcagacaataatcactatttgaaaagcggacaaatacatgctgatttttaaaaaaaattatagtagtacagaaagatacaaaaaaaaaacaaagttaaagtcttcctaactctcctgcctgtaccctccactgggcaaaagtccctgtccctaaggtaatatctgttgacagttctccttgcaatacagataggagcatgttgtgtatctgcctctgtgtgtatgtgtttacatgtcttttttttttttttttttttgagacggagtcttgctctgtcatccaggctggagtgcagtggcgtgatttcagcatgctgtgacctccgcctcccgggttcaagtgattctcatgcctcagcctcccgagtagctgggattacaggcaaccaccaccacacccagctaatttttgtattttttgtagagatggggtttcaccatgttggccaggctggtctgaagctcctgacgtcaagtaatccacccgcctcagcctcccaaagtgctgggattacaggcatgagccacaactcctgcccctgcacatgtctaacgcacacaaaggggattctgctgaacacatttttttgtgctttttctttccaacttaacatatttgacatctttatcagcttatatagctttacttcattcttttaaagaggttattggctataaagagaaagtcagagaaagtcagtcaatgggtgcttctatgattatttaattagatcctgttgatggacattgatacaatttctaaattttttagtatcctaaataatcctgtggtaaacatccttatacagatgtccttgttcgctcatgaaaatatttctggaggatggtgagaagggaaattttaaaaattatttatataaacattatcatttgttagtagaatgacctcgggagaggttgtagtaatttatgcttccccacatgcatatgaacgccttttcaatatattgtttcaaaattggatatcatgaacctaaagatatatatatatgtatgtattttttttttttttttgggggtggggggatgtcctgttattttcagttatcgaatagaactttgttagttccttcatgtaaggatgaagttggtaattattattttttttgtgtgtgtgtgttactttctttttttttttttttgaagttggtaattaaagggatctgacttcagttatggaactgggaaaacaggaccttgatgtggaggtgggcttagacatgctatgtctgggcaggtatctcttgggaagcagtgtcacccctgaacagaagcatggatgagccgcaggggacggtgctgagcagaggggctggccgtgggtcatctgcggctgttgacctggaaggcacaagggagttttccacctttcctttggttttgagagtgcagaagctactaagcaactcacaacgtgcccagggtggtggtccaactgaaggatgtgaaacggttcttcctttcccagccaaagaacttgaacctcccaccctgtgacaagcattataaaattcacataattttgataggctggattccctttctgtagcagatctttcctcagaacagaagtggttttttgttttttggtttttttttaacctaaattacctgtgagttttattttttaaatatggaatatgttttttggacacctccttgtcatattaaatgttgttattaaatttgagattttaatataaattttaccccagcaaattaattttgtttctcttactctcttgtttttggcatcttttcccgttatatagtggtgctcatgtcatcatattgctcttatgtgacttttcctttgtgaacagggatcttgttcacttttccttttttaactttttttttgttttttcgagacggagtcttgctctgttgcccaggctggagaacagaggcacagtctcagcccactgcaacctttgtctcccgggctcaagcgattctcccaccttagcctcttgagtagctgggattacaggcatgcaccatcacgcctggctaattttttgtatttttgtagagatggggtttcaccatgtcatcgaggctggtctcgaactcctggactcaagcgatccacctgcctcagcctcccaaaatgctagggttacaggcatgagccactatgcctgagtcacttttccttttaacataaaaacactggtatcctagagagggcactatattttgggtacatatgagttcagatcacagaagagttgtattctatacttcttttttcatcattcttacttcagtatgatgtattttataattttatatgagaaactataacactgggcatgtgtcatcaagcagtacctactcataaatcatattaaattttgatccaaacatgggacaaactgaagttttctctgtgtacttgaatgctttcagaggcataaaattatattaccatgtgaaagcaagcctacaaaattcctcaggcgtccactctgccactcaaatgagagccagacttacagtgcacactctacaaacaaacttccagcccgtcagggtattttaagtgcctgaatatgcaaggcactgtgccagtaaaattactcagtcccaaggatagagcctgttagaattattttaaaatctgtacttgaagtttatttcctcagtgttccaagatattttatctggttgttctctgagtatttcacatgtagctagttacatataggtgagatagtgatgcttgtgcctggtgtggatgagaaactgagcctggcagacctgagattggatttccttttctgactttgcaagtgtggcttggctttaatagccctcctctttttccgtattcctctttcccccttccattttgcaaatatgcatcaatcaaaatacaaattgattgcaaatatgtgtcaatcaaattgtattgtattttgattgtgcacatatgcactgatagttacacatatgcatcaatcaaaatacaataacctgtggaaccatttttttctgaaataggaaggtggtgccatgggctattgattgttgaaggtagtcttcagagcttgtcttacaaatctataataattttcccccaaattaatgtgctagttagaacactagattgcatcactgaaagtgaattttagaatcttttccaactttttaccaagttcagaaaaccgttttattggacctttatttgcctcatggtcatctggtttctttatgctgataatgacagggaccttactaacctacaaggcaacccagtccatcctgggcagctatgatcattggaaagctctgaatcattcatctggaagctgcctatggcagaatagtgcagaacatatgcattgcactatgttctacaaaactacattaaataagtgttccttttcattgttagatggaagtgacccagggcaaggatcatgtttgatgagccagttttctcctagtgccttgaacataggcacttggtaatgtttaaagaatgaattctttttttttggagacaaaatctcactccaccacccaggctggagtgcagtggcacggtgtcagctcactgcaacctctacctcccaagttcaagtgatttttgtgcctcagcctcctaaggaactggaattacaggcatacgccaccacgcccagctaatttttatatttttagtagagaaggggtttcgccatgttggccagatggtctccaactcctggcctcaggcagtctgcctgcctcagcctcccaaagtgctgggattacaggcatgagccatttgcctggccagagaatgaattcttaatcagtttcttatgacttcatattgactgacagttgtgctaaatactttttgcctttatttgaattagaaaatttgtaagggggccttcagaatagatgcagtcacatttaggagttttcttccccccattagttaaaatgcagtttttctatggatctgttttagaactacgaataaactctgatcagtcactgcagactacgcagtaagataagaagcagtccatttgttctgtcgtttattcatggccctcatctttgccagctgacttgtgcagaagagcacagcaaactgtaagcttcctaacagctattcccattcagctgctgtcttcaagaagcagaagaggatagatagaccttagagaacataatcaaacctgggaaactcaaatcaaaacaaaacacaacttcaaaacagaagttgaaggtcctgaaggtacccagaggcatagcaaggaaaatggccccactgggatatcttttggattcacctacagacctctctggagttttaagatccactttttgtgacatattgcatagcaaacaggccgaacgctgttgtaaatcatcctctgcgaaaggcccaggttttgaaactgaaggcctgaaaaggctcctgcttaccagctgtgtgctgtacctgacacactgaagcctgagagtgccagtcacctgtcagagggaacacagctgccctcaggcagaacctgagtctagaactcaaggtttttgactcttaggctaaaaataaaaaatcagaaggaggaactatggattgcttaatcagtcacattttcactttccagagtttcttgtagcacaagttatagcctttggggattaggatgtaaacacttttttttccttccctaaagcgatttttggtctgtcagtttttccagcttcctgctctgggccctgaaggtgccacacagttgcaggcatgctcctacttcaaggctctgttaagctgtggctgcccttccttcccactgctgtgccccaggccaacccctctgccccttccttcccctgaggcgctttgcacctgttgctccgagcctcctggtctatcccagttagtagagtccctgtgcttacctccgtggctcagtcctcatgggtggtcagtgctctagggaaggtgagctgacctcttcactttcccttcccagccatttatagctcttcatagcctcaccaacttagaaagggatgctgcttttctctctgtcccccatcctgactacattcaagtcattgctcagcccagtgagtttgtcctttcttccagaccagtagttttcccccaggccctcaggcttcagcccttgaaggcatccttagccccccccccacactatgatagccaggcgctgtgcctggccatggggtgtgggtatgtgtgtgtgttttgtttctggagcccattcctttccttccaccatcttctaggccttcatccaatcctcctgcaacttgattcatgacttttccctgcatctagtctagtctctttgctggtctgttgtttgcatagttgccagtttaatcttcttaaaatgctgcatttttgaagtcagtctctctctagtccctgtcagaaacatttaaatagctctgttgccacagggggaaagcttgaacctcttagccagtcagtcattcagagcctgccctctactggggcctgctttcctctccagatgcttctcccactctgcctgccacagcccttgtagctggctgattgccctgtacctcgcaggctctggaagtcttcagctccttcatggagcctttgtctaccagccattcttttcaagactttgctgttgttcactgcctgatttgttcatttgacacttaattcactgagcaaacattatgaaagatgtactgtttgctactgtgaggaatggatccccaagaggtaagagggtcagtccctactgccaggaaatttgcctggattcagtatcttcttgtgtctattagatcagaagtggaaaggcagaggccaggctgtatgctttttaaattttattttattttatttaaatcaccagcacctcagcaagtgttttgcacaggaaatttcttaatgttatttaattattttggtttttttgattaaattctgtacattcccattttagcttatcttgagttataacattaaaattaaggtagtcatcaactgaattataagacctaattaaataagattatttaagatagtgatttctcattagattggccctattcatattaacttttctgcttttttcttcagtctgcatgaagaaatcagtgatttttatgaatacatgtctccaagacctgaggaagagaagatgcggatggaggtggtgaacaggatcgagagtgtaattaaggagctctggcccagcgctgacgtgagtcccttcctgggtagcctatgcttgggacagtccttgtccacgggccagaggcctatctgctagtatctcatgctagtcctcacatgcaagtagaagtgcactgtagagttgtggtctaattaaattttaaaggcaaagaattttctgcagtctttagaatttgaggcttactaattattttcattggattggatgactaacaacctttttttttttttttttgtagtgctaatagcaactactaaaggcaagctattgttagaaattattagtgtaaagagaagaaagacaaatcaaacctcattgttgtagtggtctgttattggatatgatatatcaaaacctcattactacttagttccagcctgccagagtaaacattatataattgtttacagctgaatgaaaatgtcaagtacgaaattttgtcacttgtggctaatgcaggcataagtcttttcttatttctttcctgaaattgccattttcatctctctcagaccagctaattgccttttagacagctcccagtcagtgaacaaaatgattgcttgggatttcttcttggcttatttgttgtttttgttactggtaccaagtcttttgtttttttttttttttttttttgagataggggtctcactctgttgctgaggctggagtgcagtagtgcgatcacgactcattgcagccttgatttcctgggctcaagtgatccatctcagcatcccgagtagctgggaccgcaggtgcacgtcaccacacctagctgattttcgtatttttttgtagagactgagtctcactgtgttgcccaggctggtcttgaactcctgggctcaagcagtctgcccgccttggcctcccaaagtgctaggattataggcgtgagccaccacacttggcctgttactggtactaagttaatacttcactttttagggcactttgagggcctgttttatgattttgtgtatgcaaagaagtaacaaaataatagaatccattactttgtgttctgtaacttttctttaggcactgtcctgggtggtgtttttgtccaattggcaaaacaaggaagtttcactgtatgtaaaacttgcatgtatatgacagtatatatatgacgctgtaggtaaagggaaaaggggaggatactaatattttatgaacagtgaccatatgtcacattctttctcctatgtgatccaaatcagtggttcttagctggtggttattttgctctccaggggggcgtttggcaatgtccgagacatttttgattgtcctggctgggtatgcactgctagtacctagtgggtagaggccatggatgccgccagccattctgtgatgagcagtataggcccttacaacaaagaattatccactcccaaatgccaatgttgagaagccctggtctaatttaacccttatctttcttaggtggaggttgactctctctctctctctctctccagccagccagccagccatcatctgtctacctacagatgaggaacatgagcttgtggttaggttcccaggtccatctcgcctcagaggttgaacttgtttcactgtttatccttttcccccgccctgagatggagtcttgctctgttgcccaggctggagtgcagtgacacagtgacatgatctcagctcactgcagcctctgcctcccaggttcaagcaattctcctgtctcagcctcctgagtagctgggattacaggtacccgtcaccacacctggctgatttttgtgtttttagtagagatggggtttcatcatgttggccaggctggtcgtgaactcctgacctcaggtgatccacctgcctcggcctcccaaagtgctgggattaaaggcgtgagccactgtgcccggcctatccttttttattacaattacctgcatacgtatttctgcctgagttcccctgttctccatgggttgaggtggaatgcatcccagttttatgccacagcacgatgttataaaatgatggtgcctggtgttctctgtggaattgacctgaaggcccacacttgccctacagttagtctgatcccaatttagtaatctattcgaagactcctgctcagagaacaaaaattaaggatttgtgattgtgtctctggataatgagggaacattattgatctgaactacttctggaagtttcctgtggttggctttctgtatccttaagtaccatacctccatattaaaccaacagtggattgaaaatattcagaaaaaaactattaaaataacaatgcactaataaaaacaatacaaattatttttaaaatatagtataatgactatttagatagcatttacattgtactaggtattataagtaatctaaagatgatgtaaagtatatgggaggactcgcatagttatatgcaaatactccaccattttatagcagtgactcgaacatcttcagattttggcatagtgggactggaaccagtctcctgccgataccaagggacaactgtattttggtctctgtgtttcatatttgaaccaggtaagttgaaattatattcagaatgtctgcttgtgaaacagaatgcccacttcatgaagaatggggttagaaaaaaaaatactcttgtcatactgaaaagtaccagtagagggtagcaaaaactgacatttctccatatcttggtgactttatctgatacctcaatataataacttctttttctgtttcaggtccagatatttggaagttttaaaactggactatatttacctactaggttagtacactcatgaatcttttaaaggactgtaccttttcctagagtgtattcgttttggctgtcaaatttgtaaggagtagaaacaaaacaaatttataaaacaaaaatgggactgggcatggtggctcacgcctgtaatcctagcaattcaggaggccgaggagggcggatcacttgaagtcaggagttcaagaccagcctggccaacatggtgaaactccatctctactaaaaatacaagaattagctgggtgtagcggcacgcgcctataatcccagctactccggaggttgaggcaggagaattgcttaaactcgggaggtggaggttgcagtgagctgagattgtactccaggctgggcaacagagcgagactctgtctcaaaaaaataaataaataaaaataaaaaataaaaaagtaaaggatttaccagcatttaatttgatttaccttgaagtagaatatcacttcacatctccaagacgtagatggtacagagagatggaaaagggatcatgttgcagtggaatcagttagttactaattttagaaattgactacctggcagagtattgctcagtcccataacttaacccactgacacagatgttaatgtagtctcatgataaaatgtctgattgtatatcctctagaatgtgagttcccactgtctcactcactcactcactctctctctcactctcactttcaaattaagaactcattctactagttatggctccagcatcctgatccagaaattcaggtacagatctcttctctgagaaagatcttggcctttcaggactcttgttcagtttcagtcttctcaaatgagacctctcttgatgcacagccttggaggctttcttttggaaatgatgtttctctgaagggtgaatacttgctctctaagaattgaaattgtttgaacattccgtcatggttattactattattacttaatctctccagaataaagtcagcgtcatgtttttccatttgagcttgatttggtacactttagcccaacttaaagtgtgctgaagtgggtggaccctggcaatttccattcttcccatagatgtccttggcctgcaaaagtcataaaatactcaacttcgagttcatatttcttacttaggttctcagcctcagtaaaacatgaaaaatcactctttcttaaaaaatttaattaaattttatgaataggtagtacattcacatagttcacatttggaaaaggcacaaaaatcaatacagggaaaagtctcagtcccacctctgccccctggttctccgtggaacagccacttgtttttttgttttgttttgttttgtttttgagatggagtctggctctgtcgcccaggctggagtgcagtggcgcgatctcggctcactgcaagctccgcctcccgggttcacggcgttctcctgcctcagcctcctgagtagctgggactacaggcgcctgccattgtgcccggctaatttttttgtattttttagtagagatagggtttcaccgtgttagccaggatggtctcaatttcctgacctcgtgatctgcccacctcggcctcccaaagcgctgggattacaggtgtgagccaccacgcccagccagaacagccacttttaccagtttctcacatatcctttcagagatacctgtgcatataaaaggacatgtgtgtgtattaacacaaatggtagcatgctggatacttgttctgcatccagtttaaagtacttcataatatttctgagttgcaattaatctcatccagataggaaaatcattatgtctagtaccggaagcctttattaaggaaaggacgttaagtgccttttttttttttcttttttttttttttgaggttttacttctttcaaaattaccctatttcctgagacctgaattctgtgaaatgactgggaggaggagtttgttaaaatcaacaactactatttcccctctccacaaaaccattatcactaacacatttagtctttgttggccagggtggaaaataggttttaattgtactaatgaagtctgtaagcatgagtgtcagttaaaacaagtttccagcttcttcagaccctcttgtatatgtattctgtgttcagtgacatcgacctagtggtgtttgggaagtgggagaacctacccctctggactctggaagaagctcttcggaaacacaaagtcgcagatgaggattcggtgaaagttttagacaaagcaactgtaagttctgcagcatttcatattaaaccttggttatttacctatgaaacttgaattaaaattaaagtttggtgagcacagttacattgcaagtgagtgattctttcattttgttaatgtcaccgtgcttgcacataaaaagttttctggttgtccacgctggattgtgaccacacaatcttgggtcactgcaacctgtacctcctcggctcaagtgatccttctacttcagcatctcgaggagctggactacagacacagcctgccatgcctggctaatttttttgtaaattttgtagagacggggtttcaccgtgttgcccagactgatctccaactcctgggttcaagtgatccacccacctctgcctcccaaagtgctgggattacaggtgtgagcctctgcacctggcctgcatttcttttaacaacagcagaatacccaattttatcacacacagtactcactgtagagatgtttgttttattcatttgcattattattttttttttgagacagagtcttgctctgtcacccaggctggagtgcatggcgcgatctcggctcactgcaagctccgcctcccgggttcacaccattctcctgcctcagcctcctgagtagctgggactacaggcgcctgccaccactcccggctcatgttttgtatttttaatagagacggggtttcactgtgttaggcaggatggtctcaatctcctgacctcgtaagccgccccgcctcagcctcccaaagtgctgggattacaggcgtgagccactgcgcccagcaatattttattcatttttttagagagagatacagtctcactatgttgcccaggctgttccctaactcctggactcaagtgatccccccacctcagccttatgtgtagctgggactacaggctcaccctaccacgccttgtttatttaaaaaaaaattttttttgtagagatagggtctccctgtgttgcccaagttggagcattttttgaaaagaactcctgatagctcatgtaaataatcatgtcagtttttgagaataatttttatatcttatcttgtcaggtcgctttttgggctatttgcaaaactgaccagtaatgcaagggggttgtagtgtataccttaagaatccagcaattttcttttttttttttttttttttttttttttttttttttttttttttttttttttttttgagacggagtctcgctctgtcgcccgggctggagtgcagtggccggatctcggctcactgcaagctccgcctcccgggttcacgccattctcctgcctcagcctcccgagtagctgggactacaggcgcccgccacctcgcccggctagttttttgtatttttttagtagagacggggtttcaccgtgttagccaggatggtcttgatctcctgacctcgtgatccgcccgtctcggcctcccaaagtgctgggattacaggcgtgagccactgcgcccagcaatattttattcattttcttagagagagatacagtctcactatgttgcccaggctgttccctaactcctggactcaagtgatccccccacctcagccttatgtgtagctgggactacaggctcaccctaccacgccttgtttatttaaaaaaaaattttttttgtagagatagggtctccctgtgttgcccaagttggagcattttttgaaaagaactcctgatagctcatgtaaataatcatgtcagtttttgagaataatttttatatcttatcttgtcaggttgctttttgggctatttgcaaaactgaccagtaatgcgagggggttgtagtgtataccttaagaatccagcaattttcttattagaaacagtttgatgatacaaaacatttaatacctagtattcattgttcttcatcttatactcagaaagtgttctccaaagtattgaggaaggtttttgttagataatttaaaaattattataactatatgtcaactaataaacgagatgatgggcatattaatttacttaactgtggtaatcacttcagtatgtatatcaagataagtatatcaaaacatgtatgccttaaatataaacaataaaaataaataatcaaaaattggacaccaaacaaaattctcaatttatagaaattacaaaatatattgtatggggcccaatgctaaatatgcaacatgatatttgtagcagtccagggttactggggtgtgccatatttagaatactcagtgttcttatgctcgcatgagatgatggagacctcatgtctagtaaccctccatcccctgattttgtcatttaatctggttaaagcatttacattttacctttcttctctttataggtacctattattaaactaacagattcttttactgaagtgaaagttgatatcagctttaatgtacagaatggcgtgagagcagctgacctcatcaaagattttaccaaggtcagagaatttagtgtttatacaataaaactattagaaacgtaattttaagattctgttgtggtggtggtctaatatttttatatgcgtgttgctgacaaacacctctctctctctctctctctttctctctttttaaaatggagctagggtctcactctgtcacctaagctggagttcagtggctggatcatgacccactacagcctcaaactcctgggttcaagtgatcatctcacctcagcctcccaagtagctgggactacaggcgtgagccactacacctggctttttttttttttttttttttttgagacggagtcttgctctgtcacccaggctggagtgcagtggccggatctcagctcactgcaagctccgcctcccgggtttacgccgttctcctgcctcagcctcccgagtagctgggactacaggcgcccgccacctcgcccggctagtttttttgtattttttagtagagacggggtttcaccgtgttagccaggatcgtctcttgatctcctggcctcgtgatccgcccgtctcggcctcccaaagtgctgggattacaggcttgagccactgcgcccagccttttttttttttttttttaagtgttggggatctcgctgtgttggccaggctggtctctaactcctagcctcaagcaatcctcctgcctcagcctcccaaagcactgggattataggcatgagccaccatgctcagcctgcaccttacttttgtatgcaatggttttgctttctttgaatctgcttgtaatgatcagtgattaacttataatgtgacctcaagtaagaattaaaagttgagaaagcttttgaagaaattgtctgctctagatccttccttgtagagacagaagagatggaattctgctacacagttgattccatctgtttttaaccttcaggagttcagattaagaacctttcctttaacccatttcctgtttgccccaagaatactgcgtgggcagtgagctgcactttcttttttttctttttttgaggcagagtctcgctctgtcacccaggctggagtgcagtggtgcgatcttggttcactgccacctccacctcccaggttcaagcaattcttctgcctcgatctcacaagtacctgggactacaggcacccgccgccatgcctggctaatttttgtatttttagtagagatggggtttcaccgtattggccaggctggtcctgaactcctgaccttgcaatctgctcacctcagcctcccaaactgctgggattacaggcgtgagccaccgtgcccggctgtacttttttttttctaaacgggaaataggttaagagttttaagagcattttctagatttcaatccctaaattacctttaaggtgtttcctacaggcttccttacttctattttgaaatgatttaagtttatttctattctattttcttccaagatagagaagaatttgtcaccattatctgtggaacattttacatacttagatagttgtcagctttctcctctctaacctaaaccattaactcctttggcttctgttagaatattcacaatttttttttctcaagcagctctaaagtttctatttcttctttgttttttaagaaaaaatgttaactacttgatgttgcaagcattatcatcttgcataaatatatggaagacagaaaagcagaaaattaaagaaatattatccataatctcactatattgggtgtgtgtatgtgtgtgtgcatgtgcgcctgtctaatatttttctggaaaaaagcttttaaaaattgaaattcgtatgcatgataacattcatcagtatgaagagatggtgtaaagtgagtcacccttacaccatagatacttagatcatttttatagaatttctcattgccagttattattttttgtgttgcttttttatgttctttaaaattataagcaaaggagtagcacattgtacacacattgtctcatacctttgtaaaaacttaatgttttggaggtttcttccatattgacgcatagaggcttgctttattctttttgttggttacacaggcagtagtcttttgtaagactgtggctgcttgatttagcagttcttcagtgttgttccagtttttcttttgctgttagaaaaagggattgacgaatatacttccatgcatgcatcttgctttacacgtgcaaaatgtttgtaggaagagtcccagaagcaaaattttgaggtcgaagggtatatccaggtaaaactgcgttagatatttccctaattatttattctttcacattctcattttctctctctccctcccccttttcttctccctctccctatccctctctctcttcctctgtccctccctttctcttttctctctttttctttcccttcctcttcttttcttttcttttctccttttcttccttcccctttcccttttccttccttcttccttttcttcctttctttcattttttgacaccgggtctcactctggtacccaggctagagtgcagtgatcatggctcactgcagcctcagcctcttgggctcaagtgatcctcccaccttagcatgagtatctgggatcacaggcgggcctaccacacctggctaccnnnnnnnnnnnnnnnnnnnnnnnnnnnnnnnnnnnnnnnnnnnnnnnnnnnnnnnnnnnnnnnnnnnnnnnnnnnnnnnnntttttttttttttttttttaatggagtctcactctgttgcccaggctagatagagtgcaggggcgtgatctcggctcactgcaacctccgcctcctgggttcaagcgattttcctgcctcatcctcctgagtagttgggattacaggtgctcaccaccacgctcagctaatttttgtatttttagtagagatgtagtttcatcatgttggccagggtggtctcaaacgccgacctcaggtgatccgccctcctcagcctctcgaagtgctgggattataggcgtgagccaccaagcccggccacttttttttttttcaaagtagagatgaggtcttgctatgtggccccccccttttttttttccttttttttaaatagagatgaggtcttgctatgttgcccaggctgttcttaaactcctgggctcaagcagtcctccttgcttgacctcccaaaatgttgggattacaggcatgagccaccaaacctgaccccttattgttctttggaagggaactgtatctagactgacttaactacaatgttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgagacagagtcttgctctgtcactcaggctggagtgcagtggtgcgatcttggctcattgtaccctccacctcccgagttcaaatgattcttgtgcctcagcctctagaatagctggatctacatacgtgtgccaccacgcccggctaatttttgtatttttagtagagatggggtttttccatgttggccaggctggtctcgaactccctacctcaagtgatctgcccacctcggcctcccaaagtgctgagattacaggcgtgagccaccatgcccagcccacaatgtttaaaaatacttacttctccatatttttgttctttcctatgcttgcttagtttgatacaatttgcaaaagtataagcttttttttttttttcttttttatagaagccatgcgtgttcactgtaggacatctagaaaacagagataagagtaaagaaaaaaatagaaatcactggccaggtgctatggttcacacctgtaatcccaacactttgggaggcccaggcaggcagatcacttgagctcaggagttcaagaccagtggtaaaaccctgtctctacaaaaatacaaaaattagctgggtgtcgtgggctgaggtgggagaatcacttgagcccaggagctggagattgcagtgagccaagattgtgctactgtactccagcctgggtgacagagtgagggggagaaaaatggaaataactagtaattttaccaccctaagtaataatagctgttaagacttctttgaagatgttgtgcctgctttgttttcctcagtggcctcagcctatggcatggcttacagagaggagtgaatgaatatgtgcacagcaaaaggtggactcattctgtacatacttgtccgttcaggtgttctctaggatagccctgcctcattccctgtaaagcatggaagggaggggtggtctgtttgtagtcatcagcccatgtgtaagtcagcaggccggattcttgtttgccccaggactgtggcagaataatctaaaggtccctagtctacagtggcgcgccaccaagaaaagtgattcttaaaaatctcactgatttagtgctttaagatgttggtgactttgtccttgtactctttctattatctgtttacaaatgaatattagagggtcatggtcacaaatgagcatcatcagttacatgctgtttgtgtttctatcctatagcaagtactcttttttttttttttttttttgagatggattcttgctctgtcgcccaggctggagtgcaatggcacgatctcgcctcactacatcctctgcctcccgggttcaagtgattctcctgcctcagcctcccaagtagctgggattacaggctcctgccaccactcctggctgttttttgtatttttagtagacacagggtttcatcatgttggccaggctggtctctgactcctgacctcaggtaatctgcctgccttggtctcccaaagtgctgggattacaggcatgagccaccatgcccagccctgtagcaaatacttagatgctattattcctgtgtacatgtcttacattttagatataaggggagaaccattcattacctatagtttacttttttttaatagcttactcttagaatggaaaattaagtatgttgtatatcgctaccaaattttataatgtaaggaccaatttatgcccctcttaatgcttagatctgttgctgatacaggaattcattgaaaatacaattttctttttcagaaatatcctgtattgccatacttggttttagtattgaaacaattcctattgcagagggaccttaatgaagtatttacaggtggaattggttcttatagtctctttttaatggcagtcagtttccttcaggtaagtcatatgggtataccatgctagtgcacactaaaagcaaaagtgatcaatcagctgggaaacattttggaaaaaatcaaaatcaacctgtaattgcattgctttccttgattccttacggtttttccctttaaactgggtacatttttatcatttagcaaatacatatttttaaattcctgtgaaagaatatttttggttttaaatcccatatattctagtatttttgagacttttcactgcaaattttaacatgcagaatgtacggcctggtttccataagcgtaaatagtataagtgccagcaataagaatgtcttctaagcagctaaatcttgtaagtttagttggaattgagatttgctatttggatgagcaaattcgagtcttagtattgtaaatgggtgtgtttatgtggcgcagggttgccaactgcctgagtctattcgtgagtcagaacgactttgctgatgtcttgggccaagccagccctggtcggcagcctggtgcacctgtaaaattcagccttacaaacagtctcccaccattcccgcaccatgggactttagtgttgtgtgtaacagcggtataggctgctgttatcccattatcaattgactgctatgctaaaccaaaattataataatattgcttgtagaagttagaatataatttattccccctctccttgataatttagcaaaaatccaatataatttcttcttttctgcttttagttacatcccagggaagatgcttgcatccccaatacaaactatggtgttctcttaatagaattttttgaattatatggacgacacttcaattatttaaagactggcatccggataaaggatggtggttcatatgtggccaaagatgaagtacagaaaaatatgctagatggctacaggccatcaatgctttatatcgaagatcctttacaaccaggtattgaaattaggtaaatttttgggcattcaaagagagggcactgtcagtcaccttattatactttaaattctctttagatgaaaaatgaaggaacaacttctaattgttactcttttttcatcaaaatatttcatgagcaaacatactaaaataaacagacacagacaatagaaaaacaccttggagacttccagataagtagggagtagaatctgtttaaccctaaaagcatagtagaaaaggcattacttatttggatggattcatgtttggtggctgcttctcctttttcttgggtccttattgccttgattataaccagttgtcagcaattaatgaggctttaatgagatgattctgaagtccttagaggcagcaagcacagtaatatatctttgaattcatgagcagaaggatgcaaggagacaatgtattttctttttgaatttctcctttcctctttgattttgcatgtctctttgtgctttttccagcttcgtgtgggcttgaaagtaagcagaaagtaaattccttccatgcttttttgaagttctgtttgcttgcttgtgtcctgatttttctgagcaatattttttcttgatataattgtaaaatatttagattcagcgttgttggacttcagtggaagtgcttttagtcatttgctttaatgtgtaaactttgaaaatgagtaaggaaagggagtgaaaagatacagtagttgcctaggaaccatttctggcttattgagctgccttataaacattaatagttctatgtgtttattcactgagaaaacattacattgattgggagcctgctgtgttcaaaagcattgggccaaaggacacgaagacttttcagcaagatgatccttgctttttaggggctcataatttagagtgataaatagatatatagctaatataaaccccaaaaatatagaagtatttctaatgtaacttggggtttcactcttaggagtgaacagggcgctatttcttttgtttgcataactgtttatgtatggaatgggatagttcttgatgggccagaatacatttcgacaactgatacaccataatgaagtaccaactgcatgatgcacatattcagagactggggagctttgggaacagctcacagctcagcttccaggcacaactctggtgggatagctatggcccttgctctcctggaagagggtcgtcaacatttagtgcacattaagcacagtcaagcttactatgttacctatatttcttttaaaggtaatgatgttggaaggagttcatatggggccatgcaagtgaagcaggcctttgattatgcctacgttgttttgagtcatgctgtatcaccaatagcaaagtactatcccaacaatgaaacagaaaggtaaaagttcatgtgtaaccagcccattgtgtcaaaattggttgtggcttcttatcttcaaattaatgttattccctccctctccctttctttttaaacacgtgcagcatactaggtagaataattagagtaacagatgaagtcgccacatacagagattggatatcaaagcagtggggcttgaagaatagacctgagccttcatgcaatggtaagatattttccttggtcgattgactgagtattagaggcttttctgtgttgtgtgcgtttaatgggaagaaacgttttccaatcttttgccactctttcaggaaatggtgttaccttgatagtagatactcagcagttagataaatgtaataataatctatctgaagaaaatgaagcccttggaaaatgtagaagtaaaacctcggaatctcttagtaaacactcttcaaactcttcatcaggtccagtgtcgtcctcttctgccacacagtccagctctagcgatgtagtaagtatgatagcctcagcccttctgaactcagacgcatgcacgttctcttgctggggttaacgctgtcttgaaggctaaggctacttcctttgcttacattttactgggatattttaataacttccatgcttgtactttttctcaacattttattatgaaaaatttcaagcatacagcaaaagtgaacaaattttagtgagcattcatgtactcaccaatagattctgccattaaccttttacttgcttatctcatacctgtctatccatcactctatccattaattcatcttattctttgatctatttcaaagtagattacagacatcagttcccctagagtactgtagcttgtgcatccttgtagccagactccaatatttgtttattgttttttcccttttttttcttttgagacggggtctccctctgtcgcccaggctggagtgcagtggtatgatctcagctcactgcaacctccgcctcccatgttcaaacgattctcctgtctcagcctcctgggtagctgggattacaggcgcctgctaccacacaggactaatttttttgtatttttagtagagacggggtttaaccttgttggccaggctggtctcaaactcctgaactcaagtgatccacctgccaccctcccaaagtgctgggattacaggcgtgagccaccgtgcccagcctattttttccttttgatatgaaattgacatataaggaaatcttaattgtacatttactaaatttttttttttaatttccctaattgttttactttcagggtctaggttacttgctcatatgttcactaaattttatcaaatacatacatacacatgtatagcccaaaatcctaataaggtacaaatattaccgtcaacccaaagagcaagctcatgattctttcatcaatccctaaccccaccctcagagggaaccactgttacgatttttttttttttttttgagatggagtctcgccctgtcgccaaggctggagtgcagtggtgtgatcttggctcactgcagcctctgcctcccgggttcaagcgattctcctgcctcagcctcctgagtagctgggattacaggtgcacaccaccgtgcctggctaattttttttatttttagtagagacggggtttcactatgttggtcaggctggtcttgaactcctgatctcgtgatccacccgcctcaacctcctaagttgctgggattacaagcatgagccaccccacctggcctttaacatttttctttctttctttttttttttttttttgagacggagtcttgctgtgtcacccaggctggaatgtaatggcatgatcttcgctcaccacaagctccacctcctgggttcaagcaattctcctgcctcagcctcctgagtatctgagattacaggcacgtgccaccacaccgagctaattttttctatttttagtagagatggggtttcaccatgttggccaggctggtcgcgagctcctgaccttgtgatctgcccaccttggcctcccaaagtgctgggattacaggtgtgagcccagctgttatgactttttgacaccatagttagttttgcctgtttcagaatttcatatatatggaaccacatagaatatacttttgtgtaaggcttctttcactcaatttttttcagcttcctggttgaattttgtttgtttttttgttttttttgttttttgagacggagtctcgctctgttgcccaggctggagtgcagtggcgtgatcttggcccactgcaagctccaccacccgggttcccgccattctcctgtctcagcctcccgagtagctgggactacaggtgcccgccaccgcacctggctaattttttgtatttttagtagagacggggtttcaccatgatctcgatctcctgacctcgtgatccacccgcctaggcctcccaaagtgctgggattagaggtgtgagccactgtacccagccaaattttttttaattgaggtataattaacataaaattcagcattaaaaatgtacaattcagtggtttttagaacatattcacaatgttgtgcagccgtctccagtaattctagaacatttccataccccaagaagaaaccctgcatttagtagtagtttctcctaattcttccttccctcccttatcctctggtaatttctaatctactttctctttctaccctgatagaattttttttcttcccccatcctgatagaatttatgtgtcaattataacgtaagttaccttttaaaatcaaggttaatttgtagtttactgatttgatatctaaagcaggcttacctgtttgattttaactttattaagtgtaggtcatgaaaagtaatctaaatattgtatgttgttgatgaccgtgtgtcaatatggaatcataaatcctcctgtgcaaaatctccccgtgtgcctttttggttcctagagcagtatgctctggaggacagaatgccaagctagatgtcacagacacagggagatggagtcttgggaagtgagagactgcgactctgagatactgggtaaagtgccagggccagggtggagacctgcagagagacgtagcattgtcatggcccaagcagcccagaaacaggtggggctcagcccactgtcgctgggaagtctgcacccacccacaccagtatgtttggttggaatgatcattgatttgtcattacagacaaatgacatcttgtgtctggctgaagccagggactaggacctaggttcctcactctttaccatactctttcattttctataaataaaaaaacaaataaactcagacctctgtgagctccttcaaggtaagatgtgggcagtggatttaacaatgaacagaagctctctgataaaatcagtcactttaaatgtttagaggaaaatttaaacaaacaattaattttgtaaattcctcagtgtgcttcttttaactcccaaatgtttaaatttagtctagagagtactttaaccaaaattgtttttctttctgaatattgagtatctaaattactaatatgtcacattataactcatgtgacttgtgttaggattccgatgcaacaccatgcaaaaccccgaaacagctgctttgccgtccgtccactgggaaccgagtagggtcgcaagatgtatccctggagtcctctcaggcagttggcaaaatgcaaaatacccaaaccactaacacatccaacagcaccaacaaatctcaggtatgtggaacgtgggtttttaattgttagtgtttgatacaaaatatttagagtttcctacatgtgaataatatgcagcatgggtttgaagaaaacgctagattgaagaacaaacttactttattctaagagattccaacacatgacagtgcttctaggaacaggatgtcctaaggatctttgtgagacaccattgtaacataaacctcttcagaaatctattgactggtccttataagatgttccagccaaactaccatataaaaaatgtttcaattgtacatgaaataagctggcatgaaggtgttgtgaggcctcatggcagtgtgcatgtctgggaataatgtatccttttctaatattttaatgttcaatagcttgttgcccgtgttgaaatgatcagctggctgtcaggcatggtcagttgattaacattagcctggacttaaaaggccacagagatactctagtttaagtttttttgttgcctagaattgtcattaactgagtaatgactcagagtgaggggaggaagccattgatatggggctctggcctgaggctgggtcactcctaactataactgggaacctgggaagaggcctcttggctgttaccttgtgtctgatttgactcactgagttcattttacctccgtggcttttagccatgtatgccagacacagactcaaaataccagttcaagtgacagggttgacagaagggactggggtcatgagaaagccacaggccatgaatagcaggaagggagcggccacctgtgctgaccccaacctacccgtgtgcctgtcatgtccaagagcctcttcccgctatgtgacttatgatgtggtatttgggttggtaggtttcttaagaaaacctttacatcccacacacactgttaagaaggtaagcgagtggtctatgcttttcaatatttccattaaaataaagtaagggtttcagagctaaaaagagttaacccctgtaacagctttttctgcccactagataagtcagtggtcagtaaggtaactctctagccagcatattcctagttttgaaagctgccccaaatccagatgcttttctgcaatactgatgtctttgtggtcgttttctgtttctgcagcatggatcagcaaggctctttcgttcttccagcaaaggcttccaaggtacaactcaaacaagccatggttccttgatgacaaacaaacaacatcaaggcaaatccaataatcagtattaccatggcaaaaagaggaaacacaagagggacgcgcccctctcagacctctgtagatagtcggcgctgcgcggtggactgtcttctctgtgcaatgatctcatgctcaggacagttgcgcagggactcctgggagacattcaggagcctcacactgttcagacgttgatttagcaactgcgttttttcccagctcgccacagaatggatcatgaagactgacaactgcaaaaaaaaaaaaaaaaaaaaaaaaaaaaaaaaaaaaaaaaaaaaaagggggaaaaaaaggctgcttatttgataagtcatatgctacaacagggtcattttaagatttaaagcttgaatgtaaaataaatatatttctcattggctttatgcagagttatagggaatagtattcagtgttggtagggtgatagaaacaatatcagaggatggggtggggaaggaaaacaaaggtatctgataggaagtccagattccaaaggggaaagtgatctgtgcatgttttgttttttttttttttaatatttttgcatatatttaccattttattgtgtgtatgtatagaagaccatataggaaattgatatttgtaatagtggatttgttaataatactttttacataacattactgtttaaattgtaaacagattttttctcaggattagtttgaaaaataatctaaattgtcatcttaacatccatatatagggaagtgattagttctattactcaatttgtttttctcagcattgaaatgacttaatagaacccttgtgtcctgctgcaaaaatttctcctctctaaagaaaaggtttatggtggcaaatgatgtttattttattttgtaaaaaaaaaaaaaaaaaaaatactatgtactttgtgtaaacactgaaaaatctctggtcatctctgagaattaacttgcaactgttttctatagtgctgtcgtcttgggcaatgggcaattacatgactttgtgtttgctgcctttgcagtctttttttttcccccccatttcttcctaataggaaaaaaaacccccaaaaaacaaaaccaaaaaaaaaaacggccacccatgtctggtctcattcctgttgcagtgaaacttcgagttccacagactttgcatgctggcttctctaaccctgtgtgctgcgtgtgcctgtttctcatctcttattctttttaaaattcatgcttaactactgtgggagaataactgtaaacagctttaattaaatcatacttataaaaaactattttcttatattccactttatgcttttggtattgttgatctttccaaattaaatggtctttgataatggatctattttgtattgccttattaagaccaaatacttcttgtcatcccattctttatcctcttctttcatggaattgttatcattaattaaaacttttttaagcattggcttgtttcaatcatactgtaaattttggttgtagtcagctttgagtgcaatgagatgtataattctgttatcattacctgttgagtttgaaactcagttgggaatatttaatataatagaatgtaagtgacatttctgaaaatgctttctttcagggtgaaagctcttatgtttagcatcagtgtgtatggctctgttaaatacagccatttctgagacaagattcttttatatatatatacatataaagtactattggcttttaggagtttcttttatatacatttatgaaatactgaagaccaatcagaccattaatggacacttagtgtaactttttataaagaaaataatgctaaagtaagaccaaaactgatgtcatcactgaaattaacaattttcaatatgttcatattttaattcacaatggaaaaatgtgttccgaaactggaaactcatagtactcgtgtaaactgtggaagattttaaatgtgatgttattttgacaatgttttaaatttttgagtcacattctgatcagaatttttatcgagatgttgagcttttgtttttgaaactagtttgtcataacattgtgcataatcacagtatttattttctaggacaattgtgaatgtgtagacttatgtttactgctaagggaacaattatttataaaataatattaaatccagtattagctgcctatttcagacacttaatacttgcagagatctatgttacatttaccacactgaagttttttttgttgttttttgtttgtttgtttttaaagaatcaccctcattgttgaaagtaaatgtactcttagggtgcgaatattagtgttccaataagcatgtgattatattaaggtggtggtagcgggaagataatcttgattccattgggaatcttaggttttcgtaaatttattgggaaaatagtttttcctgtactgctgaagtttctttttggtaaacagtatctttctaaaagaaaaaagcatgaaggagaaattgaggtgtgtatacatttcctcaaatgaccagcattgtattcgtgaatactgtgtatcttgcagtgaacagtgtggaagctgttcatttttcaatctgaagtaaaatactttcaataacttttagtttgcctgctcatttgttttatacatttcatctctctatttgactcctatcttacttcttttttgagttttaatactttctataaagattttgtgaatatatcagaaatgtgtcatttatatattatagtccattcatatccatgaatcataaccttcctttgctaatacttgtagaatgggattttacaaattctgcctcactctggtgacatttctctggcagtcatgtatgtgtacctggccattagaaatattaatatttaaatactgttttttagaggtgctgatgggttggtgaggtgtcagcacaaaatcttatgggttatgttttatgataaaagtatatccattttttccctccagctttaaggtgactgtgaaggtgcctggttttgaacgtctttgtttggtttggagatgttgcactcagttttcaaatctagcttggatctgtaggacctatgttttttataagtaattgccctccagtcttcaacagttgattctgttatatttttggcctgttttgagtgtactttacttgcattttgagccttattaatatttagcttatttgatttggctccagtattcctagatgaaatctgcacagggcaaaacatgggcaatagggtgagcatttttaattgtctttttccactggaaccttatatatctccatgtgttttctgctcattccctcccccatgaaatggtaagtgtacttgtgtttgcctgaacctatggactagtgtttggggtttctggaaacactagagggtcagaaaagagtaataaccacgtgaagtgcaggattctcttgctgtgacgtgttcgttgcaaagccctctccagcgactgggaggtgtagttgttaaggttgatctgttagaaatcaccattatgaggtattagtggtaaatgttgctgatatttttattggtcatgactacatctcagttttactttaatattgatctatagtttgatcagttccttgaattctaacatgttgatttctcaatgtttctgtcactaaccaagaatgtttctagacagttggttgcttcacagtcaaaattaaatggtaaactatcaaaaagacattcccaattttgctgtgataaatattgaaacattaaaattaatgaacagaagaatttattcttacccatctattcttcttctcctagttcattacactttttcagttactggaaaggcacattctctaagtatttggtgagcaaaatattctataaatgcctctaacaaacctaattgaatataaaagttacatttagtagttactgttgatagtaattttcatcagggtcatagttcatctagtaaaatatttagagaatgacgttaacattccagcattaaagtgggaacaaagatttgtatgtgaaattccttgaaagagttcatcttgccttggtttctgaccctcaagactagctacctgccatcttgtcagaacatttgtgggtagaataagtgttaaagatcaaattttaatgtgctccttgatatttaacatagctaagaagccagattttactgcagaagttatttacatgatttgaaaactcgacctaactggaagcctttttctcagtcatcttgttctgagccatcttgacttcacaccattagcaacttttctttttttttggtcaaagataatgagctaaatatatgtagatattgaatgttgacaaaattattaaccagaaaaattgcttataaagtctgctgatctatttgatatctagaattaaatatttgaggacagtttttagttagtaaactgctaatgtttattttactgtctctcaggtttttggttttttaaaaaaaatgtttggcctttacattttctatttaagtgtgtactttattgagtttaaccttgtccatagcctagtagcctgaaagaaaaggaggcggaaccagagagatggatgtagtgcattcactttggttattataaatttgtggtagctcctggatttactgagagatattttagttatgtcaataagaacagctaatgatgtggaaatcaggtgttctcttgtgtatttcagtgaacgtttttattagtatttgcatatcatctctagttctacgttttaacttaacgtccttgtggcttcaccactgaggtacctttcactacaccagcttctgtgtggcctggtaacatggaaggtctctcctaaggacagtctggacatattttgggggaatgttatttatcttaaagatgcctagaaacaacgcatatagtaccagtgagaaagtatgaagtaaacaagttgctcaggctgggcatggtggctcacgcctgtaatcccagcactttgggaggctgcagtgggaggattgcttgaggccaggagttcaagacctttgtctcttaaaaacaaacaaaaacctgaatggtgaggtggggtagaattgggtaggggagggaaagggggacttggaaaagcattccccaaagccagtgacttggtgaagttcagtacttgcctcttagaagttagcccatgcctttcaaagagagtgaaatgatgggttatcagccacattcttggagttaatatgtttcttcatctttcaatttggattctgtgctattcatagctcttccctaagaccatttcattattacctcttatatttagttgcaatttatcataatatgttgttttgtccctaaacttaatctcctaattttaagatcctctctgatttttgcatattgaaatttgcagaagtcacttttaagaagtcttttgaaagtcttgcaatcctaaaataaatcaagcttgtttgttagaagtgtcatgagtctccagtctttactattaaaaagcagccctgccttaacgcacattgttatgggtgacaagtgacggacaacaagtgtagtttctggatataaagagtgaaggactattgggttaaacatttttagtggaatatacatagatatgtatatttagaaactttggtgaagccagtatttgtttttaataaccttttcatttatttccttctttgattagcattgtcttctgtgttaagaaatgtggactcctgtgaggtgctggaggtttgaatcatcttgaaaactttccagtcttgtctagttaccactgcagagacactaaggaatttaccagaaaaagatatttgatacaaatgatttaagaaatctcaacatttcctgaggccatatcactgggcaaccagtgatgaaaactatgaatgaattgcacacctggaagattttttaagctaacgacagtttcttcaaagatgtcaattatttgccttggaaattttataaattgcatttctatgcacatctgcccctagtgcttaccacttggtttattattcataatctgcaattcaataaaggctttgtgctttcatttatcttcaaaac

SEQ ID NO: 4 - DNA, 49960 bp, Macaca fascicularis
  source 1..49960: mol_type = unassigned DNA
aactacctgctgtccggcagccgcgcggctgctctcagcggagggggcggccccggggcccaggcgccgcggcccggcaccccgtggaagagccgcgcgtacagccccggcatccaggggtgagtgcacggggcggccgcgggggcgggggcggggaccatggtcctggccggcgcccgcgttgcagacaccggttacaggcgcccgggcttcttttggaggatgcatgttgaaggccaaggcccgactctgctctgaaagttttttttgttttttttttttcttgctaatatgaaactcctttataatggagtgacttgcccagatcctgcaagtaacaatgcaagaaaggggctgctgaataggacctgtaggtatttgtcttttttactcttgagactggaaagggaaatcgactctccccctgcaccccgcctccgggcaagtgaggaaccccttgttaaagtggggcgtagataagtgtggagtttcattaagttaagttgcagaataatttagcattaccaggaactcagatcacgtcgaaggtaaatattaaccgtttttatttcatttaaaacaaaaatttaactgtcaatttagaggtgattcattggggggggttgtgtgctttaatttcgtgctgcagttaacataagcatagtatatacatattggcttaattcaaagaaaaaaacagatatgtcatatatatctgttctttggaagatgccattattattttaaatacttccacatccgcctggagggaattagaggctctacttatatttagtgcacctacagacggcaaggaatgaaagcaaaggtggtgtgtgtggggttggaattgccccaggtgaggctgttcaggtgtgatgctgttgacgcagctctttggccattttgggcttttctgagcgtctggaaataatttatgtgtgggttgtatgtcagtattttaagacttaaacgataaattttccttgcacgatttttttccccccaatttaaaaaagaaggatttgccggggtatgagggttgttacatgcagtagagtcctacgaataaccacaattgctaggcgttgggagttgttacagtgcactttttcttgtaactctgtttctcatgtgaagtatgttgggaaacagagttgatacttctttaaagcgtgtgatacactgtaatagccgcatgttgtgtaacttttttcacctggcttgggctacaagtgatgccttctaaattccctgaaggtgtaaacatgcattgcagacactggcgggagggcagactcctctccctccagttcttacctgtagaactttctgagagcaggtggttggaagcggtttcttcttgatgtatagtaatgtatacctcagggctcgcttgggaggacctttaggtttccagcctgttatggaactggcatgggctctttctagtgtctcttagcactagaaaaaacagacacgtcgtctctgcaagtcttgggttgtacctctgctcttagcggtagcgcatcaggccccacgcatctcacttggactccccaagctgtttcctcctggtaactagtaagcctcagtaggtcgtgttgcttttgttagtgaatgagcccactagggtcaggtgctgtgttgggatctggggatctggtgcagagacgatatggagacaatctcatgatcttgacccaccctgaaacctgcctacaagtgccccctgcctagagcacaagagtaccctgctcagttgtgcaggtcccctctcacgctcttcgtcacccacgcccaatcaatcatcagatgctgttcgtttggcccttttaatctctccacacctcatcctcaggcagtctcttgttacctctgtggagatcgctcaagcagccggaggtgccttcgatgcactctcgtcctgcttgagtcctttcccatcactgctaatggggtgatcttctctgccggcaggtctggtcaggttccccttcaccctctgatgtcacctcctccatctccaatcccccacattaaagtctaaagcctaaaactcagcaacacctccagggttctgctactcagatgaccctgggtcctggcccctggccccacgccccaccccttgccaacctgttagaacccagctgtaaggcagtatggtgtagggcataagagagggctctgaagcccgtcaggaggtctggtttgatgctacagtcatgctcttggaaccttccttttcacctttgctggagacctctccttgcctgtctccaggtggcgtgtaccatgcctccagtatggccctggtgcatccacttcctgctgtctctgcccaaaggggcccgtgaggactacctgctatgagcctagaagggcacgttgacctctgcttgggctgctactcaagctgctctttcagaagtaaacctaagcggtggtagagttgggcctatgactagaggaggtaaggtccccccagtgatcatttccacatggcccgttggtcctacatgaactaaccttttcgtgtcaagaaatagttccgtctagaactttcttctggccatcagcttacccagagtgttgagaaaggccaccaaaaagtctttcagttgtgccttagagaaggaaataactgagttttaaaggcacacctgagctgaccaatagtaaaggatcttgttgctgggaagctgcttggggttgtgatatagtcccaggacgtgcacttctgaaaatgcagtgtgcgttcctcatgggaggatgagcctgctgcggagcactggctgaacccagttgggtctttgcctggtagcccatgtggcaacccgcactatttgtccttttctggggaggagttttctgtccttggacactttgcctggtggcttggccttgtgagactgccagtctgccttctgctcaagtaggatgaagaaaaagcaggtgaaagaggacagggattggtgcaagaaccttcagaggagaggaggtgaaatgctccttttgactctggttcttactccattgtttgattgaaatcccaagcctttgttttgggaatggtgtgcttagcgtgaactctgctgttgacctgggcttctgaccttgagatgttgggcaggtgtgtggacaggccctgggcccgttgcatcccagcctctggctgctgttacttgcacgtgctctcatgccctgacccagcagggctttgagctgttgttactttgccatggtcattctagcagctttggaaacctctccaggttaaaagtcttgcgtaagtgagagtgggagcatggccttcagatatttggccacatccttcctggtgtgttacagagacccaggaagagtgtagttgaacgaagacatgtgcaggtgtcccgggccttgaacagcttctattcagagtttggccttgcgaaggctgtgccatctcaggtgtgcctgcagtgtgcagcaggtgtttgcagcttcctctctgcaggtttcttgatatttaatttcatcttttaatatcttctgttaactcaaaggaaattcttagtttgacgtatgagagagactgaccactgtcagcataggacatggtcagccgtatctcacaaggcccctggtgagagtcgtttccaacttggtgcatgtttttttcgattctttcttgcaaaggagtcagagcttggaggcgcaaccaggatcccctcctctccctctagcctggcctcactgacataaagtagagcaggtgtgacctgtctggaacgtccttgtgatgctcagcagggcctgctgcagagcacgggaggcatcactctaggggcctttccctcccataccttcctgtgagtgtccaggatacatagaaaggctgtcgtgagacctgccgtaaaggatggcggtggcacgtgtggaccttgctttctgagtttcactgtctgagtcccagaggtataagcttgtggagagaagtgtacatgatcacactaagcagatacttgctcctgcactgttggaggaggaggagggattaatttcataatttcataaatcaactcttccaacactcctcctgtttagtaatagcatcacttgttcctgtcttttgttccattgccaagctccccaaggtgaaattgaaaatagagtgcaagacacaggctgtgttggagttgaggaaagttttgctggagaactgcttgcaccacgtgtctggtcactgatagatgaggactggccaggtcaggacagctgacactgggagaaggggctgcccgggggggcatgacagactctggaaaaggagggttggagtattaaactggctgggaatgagaggcctctaatcttttctccaaaaagaaaaaaaaaaaaaaaaaaaaggcttaatgctcatgcggtggaagtcagagtgaagcaaagtgagttctgtctgtcttgctgctatgagcgtgtgatggaacaacagtgtcatttgctttttctcaaatatttaatgcatgtttgtgacataatttttttaagtaatttcaaataaatattttaaagtaaaaagttctaagatttttgtgtctccaggtaaagtctcaaactgtctttggtcactaatatgagattttgtcttcattttaaatggattcatgtaagtgtcctgtgggagaagagttaatggttatccttggaaaataagaactttttatgcctcagttaggtcatatggtttaggatctgattttgtagttgtggagtaaaaaaggttgtttaagaaaaaaaaaacaaaaaaccttgatattcaaattcagaaacttgatttttgaggatgcaaccagaatttggactaacggtggagagggctggcggagtcagaccacccagactcgcagaagattgaagaagctgcagtgctcctgtggagaggccctttctaggaggtgtggctggcttgtcagtgctttctcttctctgcagtgaattggatggcaagcctcgccctctttgaaagctgccactctgagcctgccttgagagcatctcagggagggcagagaggtggcctcggtcagcgctgacaggtgcccaaattacctgacctgttgtgaacatgtgcctgagtgaggtggccaggatgccttttctcccaacactggggatgtacactcatgcaacatcctatttttgagatttctatgttgtggtagttctctgttgctgtgtagcaaattagcgtaaactcagaggcatagaagaacactcatttatggtcttgtggattctgtgtgtcaaatcagggatggctgggctgggttctctgctcaggcttttgcaaagctgaagggtgttggccagctgtgttctcagctggcactcagggtcctcttaccagcgcattcctgttattgttagaattcagctccttgcaggatcgaagtccttgattgcttgctagctgccagcaggggaattgggtagggctctctcagcttcttaaggccacctgcattccatctgcaaagcaaggtactttgaatttctctgacgtttcccgccagctgcttagaagcccatggaagccccctgcttctatgggcttgtgtgattgagtcaggctcctgcgaataacctccctacctgaaggtcatgtagtaatacaacatgatcatggtgtgataacctcatcagagccacaggttccaaggagtagggtgtaggaccttgaggggaggaggttcttctgtgggtagacttcccctggcgtggaatcggttgatgaggaggttgtggttcttcttgtccaacacttttcccctgactggactccagcccatcgcaatgactcttgcagattgccggttctgtcctctggcttggtggttactgctgaactcaggcagccaccataaccaggagaacctttctgtgctgcagtcagatggacattctttaaaatatgtcgtttaagaaaagtttgcaggaaagccgtgtgaatatatgaaactacggtgattgaaaagtcctgtttgtgaggtgtcctgcattggctggattaggaaaggggtcattcctatgcaggtgggggtcgatgacttacccaagagtcacctctggaacattctcgtatgttcatagcagtccatctttgcagaaggtgtgggggacacttgtcctgcaagcccaggttcgtaggaatgttagcttccccagacccttggccgcagagcaccgtgcctcctttaggagggacaagaaaactcccatatggttcttccctctgtcccttcctgagcccctctgagtgtttggctcttgtggagttactgctgctataacaaagctgacccgactgccctcctgagtctcctcagggaaggaggagctgtattgtagcctgcattcttagtgccaagcacacggtagccactaagtatgtatctcccaagaaagaagagcagaaagaggatctgccagctcaggagggcaggtgggggatggcaagtctgcggagtgttcataaaccaaatgctaagggaaacttttgttggttgtcttagaattttaaaaaataaagtctgttgcagtttatctgctttccttcctggagagtggctaaactaatgttctcttttacaatatagaatgcgagagcagaaaacattcaggaaaattctatactgtatttgaaaaacatcaccatttagttttaactgctcgctcttattataaaaattaatacctaactatgaaaagttagaaagcctggagaagtatggagatgagttgtccaccattgggccacctagagagtgcactgtgagcctgtccctggtgtcctgtactctggtgatgtgcactgcatttgctggcagtgagccagggagtggaccacatccgggcctgggccgggtggatctctgcagaagtttatctctcggctgacagggtgggctagataggagcagctctgagggtccctattggcagggaatgtttttgattatcaatgacatgcctttttgttgtgggcttgtgatctttttctttcctaaatcagctgctgtgcataaccagttaggctctcccgtggcttaagattggaattggtttcccaatgctaggatgtgggtgttaggtggtttctatctttccgtttgaaagaggtttcagatcagtgtaacatttctagaatcctgttgatctgaaggaatggtagaatgtagtagtttaagggaaatgaaaagttgaattattttgatgtttctgctttaaacctgctgggtggagtccatttctgagcactgggagccacgtgcttggctcttgagagcctagctcgatcagcccatgtcacacactcacaggtctggctttgcctggtttcgccacggtttctaacttgagcctcagtttccccaactgtgaagcggagcctgtggcacccaccttagagcacatgaaagacttggaagaggccgggtagcctgtaatcccagcactttggaggtcaggagatcgagaccatcctggctaacacggtgaaaccccatctctactaaaaaaatacaaaaattagccgggcacggtggcgggcgccagtagtcccagctacacgggaggctgaggcaggagaatggcttgaacccgggaggcggagcttgcagtgagtggagatcgcgccactgcactccagcctgggtgacagagcgagactccctctcaaaaaaaaaaaagacttggaagcactctgtgagttgtcatgcaagagattaaaaaggccaccactgcccttttctcctctctttaaggaaattgaaaccaaaaattaagtccttcttcttgccagctggacaggaaaagcctttttcttggttttttgaaaatacagctttcactttcagatcaaagtgaaaactgctaaagactgaatgttctgagttgtgggagtggggggctggaggggtgttatgaattgaaagatacttttctatttttaaaacattttaacaatgccttaataatatctgttctagttttgtgttttttttttttttttttttaactgttctgaaggtacatcagcactgttctacagctttaaataagaatctcatctccctagaggcaagggtactctcgatgtatttggtcaggggtgaatgtgctgctctgtgtaactcgtttaaagttggttaaggttttttttattttgtgcacatagtagaaggagtgaatgaagtgttttctgaactcttccagcttttaacatagtattctgtgtatagcgaaagaaaacaaaattaagggccagggaacattaagaaggcaactagaacagcactgtccagtagagcttcctgtggagatggaataattctagttttgcactaatacagtagccactggccatatgtgacttttgagcacttgaaatgtgacaaatgcaactgaggagctgaattttaattttatttacttttaattaaaatttaaatggccatatatttagacagctctgaactgcagtaactttagactctatttgaaacagcgtttgattcagtaactgttgctgaaataaattgaaactcatacaacagtaaaaactgtgttactcagcaagttaccactgtgaaagctctagaaattgtttgaatttccaatgcaaatccttttcaaacagccgctgttttaatagcacatgaagcataaaataggtgcatagcagcgcaacacagagcaacacggagaaactattgtgacctggagtgtctccagtccagcatgcaggtgataggtctctcagcgtttttgcacacaggttaatgatggtgcagacggtcacttccttctctgaacagccttcctggtcaccagcaccactgtggctcatgtgtgtgaggctcttttggcttagctctctacgtgagtttgctccttgagttcccagagctgacccatgaaatgagtgatattactcctgttttgtagatagaaaactgaagccttgacaggctgacatgacctggcaaaaggtgctgcacttggaaatgtcaattcacggctgacatctggacactttactcctaactgttcagtgaacaagacgttgctaacatgcgggggatggaacctagcaactcattctacaagtatggttcaaatatgttggtacagggccttttgctttgttttcctaaagagattagattgagatgtggcggagtgctttgacagccaccgcaggacaaagttgacagctctggggttggggagtgttgaggatttcggagggaagccagtcctgggcagtagtgcgctctgggttcgtgctttctcgaggtgttccagagctggcctggagggaggctgcggtgcggcagcgggatttctgtcacctggagtattcttagaagttgcattctatgaaaagtggagcatctgatgagctgtttactcgctgttgtatctgacggcagttgaaagacaaagcaggactggcagcgcagccgcctcagtcagcactgctgcgttgggggcttgacctgcagtctcgcaatcctgggcaataactcattttcaagaaagaaaaattaaacattaagtgattgaagcgtcttgcccaagccgctactagaaaataaaggtgctggtgctcaggtgcaggtctgcatggtgtgcaaacctgggctccttcccacaccccctcagagagggcactggtatgttggagtgaagagccacgcaagacctctgtgaatgggcagagatgggcccgtggcgcaacacagtaaaatgtattttggttatgggcattgtctctaaacttatgtaaaacattataaaaaatggaaggacaatgatgaaatgatggccaaaaacatagaaaaggataccttgcatgtactgtgaaatgcaaagaaattctaaagtgtcattacgagttacctcatggaagaaagcaaaaggtgaatctatctagagtttgtggttctgactcacaagagactgatgttcatgctgaaggacaagtgtgatgggtggaaggatagagcgccaagaccacactctaaagatgggaacctatgggaactgtccagggagatgaaagcatggaatgaactgaagcttgtggacttgttgagtagaaagagccttttaggattggttttagaataaaataataaggcctgtggttggggaagatgacttgctgttcacagagcctcccttaacaggtggggacctcagcttttcctttgctgccatcaagtgagtggtgtacagtcctagccacagtagtaatctctactggcctgactgagccctcaccctttatgcagtgtctcctgccaccctcctgggagaggctgttctcagcatagctggcctggggtcacacagctggtaggtgtaaagctggcgtcggagtccaggtagtctcactccatagcctgcctctttagccaccgcagatgtagagcaaagcaagaattgtccaaagaggtaagctaataaagaggaaaacaggctgggtgcagtggctcatgcctgtaatcccagcactttgggatgccgagaggggtggatcacgagttcaggagatcgagaccattctggccaattctctgggcgtgatggcacgcacttgtagtcccagttacttgggaggctgaggcaggagaatcccttgaactcaggaggcggaggttgcagtgagccgggatcacgcaactgcactccagcctgggtgacagagtgagactccatctcaaaaaaaaaaaaaaaaaaaaaaggaaaacaattatgctgagttccagtaatctctgggtccagagaagtgatatctgacttctcaggagagatgctgatgctgtctgagggcctgcccagtctccgaatgattcagacactccagggatggacagttagtgccctaattttcccaagaggattctgagggtgacttctctcaacccaacaggaggacctggtgctgtcatcaccagttggagagaggctgttttgtgaggctggggaccacctgctgtgtgtgccgtcttacactgccatttgctgatcgctttagagctagaggttgtttctaagagtacttcgtgctaactaaaaaatgacgacattgtttttataaaactgattcatggtttgttttttgaagcagaaagctctgaagtcactcagtcccaccacccagaaggacaattaggtgactgtcactataggacctctctaggctcgtgtagatggacacatggattggtcagtagaaatacttacattaaaagtcttatctttacataaatttgccaaattattaattttgcttgaaagggaaagatgtctaacttcagttggaaatactggtttctgagagatagtgctgaggctaagaacataaaacatgaaaaccgtaagtggctaagtgcagaaacatcagagtaaaatctttgatgaagtcttggtttgcttgggctgtgtaggttggaggctcagcctttgtttctccctcctgtggtgcccagtggtggtgtttctgtgtccatgaattttcagcatctgtagtttgtagttatgaccactactgcacctagcccttactgagggccagggcctgtgctaagcacttcccagtgtggattcattaagtcctcataacagcaccatgtgggggcagctgcgtccctatgtcgtacttgggacgcgatctgaccccagtcacttcgggctcctaaagggtctgcagtctcgcctctgcctgggaaaccacgatttgcagcatattccacagacctcatgcttatcaccaggaggcttcctgggactgctgtctgaggtttctaagttcctaacgtggttcatgcttctggtgagtttctttgaggcgacatacccagcattgcctctggtcagctcagccctgtggtcccgcgtggcatcctgcagtgtcctctggctgtgactcgtgccgcgccaccttgacctggcatccactgtcctccactgcgtttgcaggtgggagccactgttggtgccttttgtcatgtgtgttgaatggtcagggtcccaaataagagctgattggggtcattctccttgaatctgccatgtgcctggaccactctttgcttcagaactcacacatgttgcctgtcctctctggcttggagggttgtgtggaatcaaaggtgagacccagtccccagggatgggcgtttgcctttgattgcccagctgtcctgagcgccctcgcttccgcgcccagctgctgcctcggttgcttgcttgaggatccggatgtagactgaggttggccttaatgtggcagggggtttctcttgagccttggatactttgctactggtcccatcttcctgtgagtgggagcgtcccgcctggcccagcctcccagcggtgaccttagctctcttagtatatgggctctgcctgccttggtccccagcttgccaccctagtgcagttgctgcttcgctcttgttccgctccttgtctctgtccaccctgtgctttattgatgtgtccttactcggtgttttctgtggagcagggcatcctgggcttcctctctgatccctggctcctgtgctcttccctgctgggctccctcttcctttccgttttctgctgtgttgccctcacacagctggcatgccacggatgtcgctcacccaagcccttcctagtgttgctcaccaaacacctcccaccttgcccccgggaccttctccccttccaggctgcaggcaggctgagggcctggcatctcaggagtgccgaggctcagcgggaggcagtggggcctttgctggcagctgggctctgcatcctgactttctgagggcttagtccttttggtccatcttgaatctcctccaggcttttggactctctgctctgtagctggcccatgggaacaggtacactcaggcctggtcttagactccactacttgggctgtgccggtgcccttggtgtcctcttagtccgtcccacctgggggccccatcctgctgtcccttgtgccagagtgctgtcctcttgtctttccacaactggctgctcattgcctttcccatctcagactcagttccagccccttggtatcagggaggctctccttgagcacccaacctaaagttcacagctgtggttaccagtttaaatttctgcctagcacctttttggtttattttggctacttatctcccccatcatgccccctgtccctcccatataaactcagtgagtagggaccgcatcatcatcctgcccacagctctactgtctgtatcaaccgggtgcgctacgcagaggctcatggtaaataactgcagaccacgaatcccatctacttgctgtgctttaatattggcttacatctttggatccagtgagttcttttctctctccctctctctcgctcagtcatacttactttgtgtaattggtgatttccagccttttgtatagtcctttcttgaatagttgttttctgtcatcttggcggggcctcagggggttgactgtgtggagggcaggggctgcagagctgcagctgctgcctgggctctcatggcgcctgcgaagtataggcaggtgctttgcctctgagccatctgtagaatggggtgacggtggtttcatcagactcagtgaagcgtgtcatacagtgagcgcctggtcacagcaggcagatgatgaatgtcagctaatgagtattcatcaccaatgaatagtaacaattttttctactaaggctatgtaatgtagcctcggaatcccactcagcacagccccctggcagcagtgcctttgagagctggcatggtcgagagaccctggttggccttactagtgtggttggggtacttgagagaactccttcctcacatagctcttccggtgtttagtctttgcatctggaggtttttcgcgattgtgggatagctttttcaggggccttgcctttggcagggcagggacgtgtactgctgcaatctggggtatgggataactttctaaaccagacccagaacttgacggccgaaggggcctcttagccatgcggggctaggagctgacacagggtcagcagcacgaggtcctgtggtgctgggtggcagagcccggagggagcccctgctggtgtgactttagtgtaaaggctgcgggatcccaaattcttacagaagacttaagacgggcacagtgatgtctgctctttgacctttgcagtatgaattagtaaaactgaaatgattacgcttcatttattaggattatgaaaacaataatgtacttattgaagaaaatgtggaaaatacagaaactacctataatttttctattgttaacatttgagcatatttcttgtcacttttaatgctgctttaaatatgtagcaaatgtatcattttgcattaaaaaaaaatgctgctagcatttcctcctgtctttaaaaagctcttttaaacaactttaaaatattgtatagatagatgtacacaattttctcaataattggagttatatttacatcttgtcactctttaggaaaggactggcctacttctgtgttgggttccttcctgagtgtgctttccagctcagtggctcggacttcaagatggagacttgagtcctggttctgtatagtcttgggccagttaccatatgtctgatgaatacttagttttgtcatctgtaaaatgaaaatagcagtacttgcctcaaagactatttgggaggatctagtgtgaatgttggtaatgcggatattgtgtagtgtcccagaatattaatgtttttagcctcttggctcttactctgtattgttgccccaaaagatgatgttcgcttcttatttttcatccagtgtaaggatatctggaaagacaaagtatagctgctttcatttcaaaagtgatcagctgcttgagctagcaagcaagctagcttccaggcgcagttatgcagtttcacagcaggcgcggttccctcggagcacccagaggtgctctgtggtcgtcagcagtgatgctgtggctgcactgccagacggggtggcaggtggaccagagcagatgtggctcaggaagtgccgtcttcttcgcctctccttaatctctttcagagtctgtgggttcttgattgcgctgtgggttgcttcagactccagtattaggagattgaaccccttgggtttttgtgtgtgtgtgtgtgtgtgtgtgtgctgagctgggttgaggacatgttaagcaggtggggtgcctcccctgggtttgctcctggtggttcctgtagtgtggggtggttctgagtagttctggccccactgctggggtatctgccgactcagtttgtgagatgtggagcttcatcctggtctggtgcctcattttcttctttagcagtgggcttagaaccaatgcagattcccaagttaagtatttttcctgtggcttaattattacaagtttctggtacctaagccctttcttactttctgttctgaggggaagaggagattatggtgtttctccccactccgagtggccccaggacctttgcatggcatttgccgtgtgcttgggtttgctctactggggtgaaagagtatcatgccccccagcactcacaaaggcacctctgctcaccctccggtgaggttctgactggccctgggacatcacctgctccaggatcctctgtggctcatcccaggagagatgtgggagagggaaggggaaaaaaggcttacacttgtcgagtggaattcctgtaggtctgagttccacattgattcctaagctgcagaacccttctgccctggctgttttgtgaatggtagtcagtcttaacctttttaaccaagttaacattggctgtctcaggaggctcacagctcctgctcctcctccaggggagtgcgccctcctcctctgtcggtagctgtcaggcgccccttccccccgcagcagattctcctgggtccttgcctggccttctccttacacgtgagcctgcagcttcattcacagccgctgtgtagaaagacaggcacatcgataggtccctccctgcccagagtgggtggaactgaggcagacactaaaagcagctgactggcagccctagaaacacgaagggtttcatttatagtttcagtccttttccttctttcgagccttgatttaaaaaagaaaggaaaaaaaaaaagccttgaaatcctgcttctggattttctaatttgtgcaggtattagttgccttgtaatgtaattaaaaataaataaaaatgatttataattagctcattaactgtatcagtaaatggatgctttaaagaggatcattgatccctcaaaacagaagcaatgcagtcgttccctcattatgctgtacttgagatttgctctagcaacccactcttcctaaagttaaatattaatccagaccctatcagtgcaacgtagtagtgtctgaatcagttgtggttttggtgtaatagcatcaaagcatgttataaaatctacaaaattgtagtggttaactccaaatatttcactgagacattatttttttgggcaaaaatgcatagtgaacattgtggagctgaggtgaggaacttcgatttctgagaaaccacccgttttaagggttttgaaggaagagttggaggagaagagggagagaataaattaacagtgagtttccagtattttctgtcgcattttacgttttagtggaaaaaactgggaactgaactcacatgcagtttgtaaaatcactttttccctagcattcaggattgatgagatttaacggggtgttaaaggtaaactgaggcacataattaacatggacagaactgtatacctgagtgtcgagagctgtgagatttcagtgagttgggagactggaagcgcccattcttcagagtgcatctcctcatgttcagtgggtttaaggtgctgaagccttttttttttttttttttgaaatggggtcttgcactggtgcccagactggagtgcagtggtgcaatcaccactcactgtagctttgaatcctgggctcagcctatcctcccacatcagcctcctgactagctgggactgcagacctgggccaacactcctggcttgaaacaacttgtatccagattggaggagggcgtgtttgtttttgtgacctttcctttcccttaaacattgaataatatcagacctttgaccatcactttgcttaaaagaagctactggatttaaagtctaggagaacgtcctggacaagcaaacccatagtatgttcctgtatgttccccacccagagacctggattataaagtgtttggtgtgcttcttctggccccactgttctttctaaagtgtttcattttacataggctatcctggcttcagggttgtcacctctgcctttgtgccccttagtacctttgtcctttacctactgcagtgcagctgcctgttcttcggtagactacaaagaagtcacttttgaacaacttagaaattgtgccttgtggggaaggcagggcagagccttggggctgagatggaggaggagccagctctgccctgggagggatacgaagctcgtgaggcaggggacgggtggagttgcttcacctctttggccccagcttcaaaaccagccagtcctccctggctttggctttaattcaagtttggacaaactggaaaaccagttaatcccattagctcagcttctaaagccgaaaatcatgccctgaaatgggtcatctgttttcatcaatagctttattagctatggaataatatagttttgttctctaactgtaggatccttcttttgctcttaaaatagctcagtaagttgggtctcataaatacatgcagcaagcatataccagataaatacgaaaacattgctgatcttgctttttagtactaaaagcagaaaatcgggaatttactaaatggagaagtcagtcgttaccttttgatgggtttggactcttgcaaggtagtgattgtaaacaagagtgatcttttgttgtttttcaatgaactttattctctaatttttagtaaagcacactaggaaataatgcttcagaattctgttttctagtagtttcttgattaaaatgaaaattcactaaaaaaaaaaactctgctgtcaccgtttccttttttcttaagataaccaggaaatgaatcattcagggtttgctccatggtgatgggtcaggaagctgcccctgctgtgtgtggggcaggaggcttgctgatgtcccaggatttcacttcgcggagacaagcatcagaggcttgcttcatttatagatcctacttcttttctacaccacagccaactcaaaatggtgacaaaatctaagacgaggctggaagtcaccgcagagcagttggaagctctgcccccggttctgggggcagtgttcctggagttgtgtccttcggccccacctcagtttgtccattcagctcctccactgagaatgagatttgtatcataaaggagtttcctgggccactcgcagcccccagctggagagtgccagcctaatgtgtcaggagtagggggcgaggccagagggctatgtgccagctccttccctaagaagcctcctgggaaagccctcccaggcacttcccaggtctcactggccgcctgagggtgctcaggccgcctctccgctggcactgcttcccgacgtgccgccagcctttctcatggggacggggatgatggcatgcttggggggcagcagggccttggggctgcctcgggctgtcagtgtgtcctactggttctgagatgccacctctgtgatccactgtagagagatatattctattatggtcaatgcatagaaatttcttcgatttagagactgaacactagtgagcagaactgaaattgagctcttaaaagattttgatgactggtctgtggatagcagactttagtgtggttattatgcggacatcctgccagttgtaaattccctggaggtttggtatgtgcgaacataagactaaacttatttctgttttgttgagaagaaatgattaaaacttttcattgatgtacttctgtaacagacttttggagaattgaagcagtgggtatattcagtatttgaagtcatagatgaataaaggaggtatgtcgtagttggcactggtggcatggcctgtggtcagcaaggcttttctgtgaaggtatgtgcacagctttctaaggcagtgaaaagtcctggcaatgttagtattgagataatccaaaacacttagtgactgtttacatttttaaagggatagttggccatttaaatggtttctgctaacagatcaaattattcagtgcagaggtaaaatattttcagaatgttaattttagatgtcattttatagagaatgtatatgagcaatgacaaggtcagcaaaatatcttagcaaaacttgattgattcattccatgcacaggcaagcactgttctgggcaccggagatggagcagtgagccattgcgtccatccacagatggcctccaggatcgtgcgggttgcagggaggccgctgggtgggcacggctgggcagtgccctcatgtgggttttgttgggctgctcttactgcgcactgtttctcagtttggtcggtgttccacctcgctgcctggcccccacccctcgccttctgagggcctcacaaagagccaagcggaaggcaggctggggactttgcaggtcagcccagaggcttcatgatgatggttggtactgtcccgcggccaccatcagtctcacgctctgcctcgtgcgcagtcctggcgtcgtgcggcacctcgctggtccctgctctcctggaaggaagggcccagtggggcgcttcgccagagccttcttgtctgactcccttacccaagaggcatggatggcagggccgccggctgtgtcctttctgttctgaaaaggcaaaacaaaattgttcttggctcttattatctaatatttgttacaatagttgctcacttttaggtccattttattacagatttcagacaggtgtggttattagtgcccctcattgtttggacacaatgccaatgtcaggaggccccgtgttcccctgagccccctttctgctaggagcacgggcaggccacgcctcgccattaatctctcctgttttaggggaggaataccaggccaccccctcttctctctgtgcaagggaacagacatttgacaaaaacggatgccatgttaacgctaattttgtttgtctgaggcagattgcagcaggtgttatctccatactcttcctttcctggagtgttgagcatgtcctgatagtaggggatgccccggagggtggtgaatgtggctgcacagggcctgaaagctatttgatgttgctgttacttcaggtagaacttaaagttgacagtacattctactctgcaggcagaaacctggagtggcattttgacaaattggaatgccctgtttatagtatgctttaatgagttaaatctgggggatgtggatagaattttagcatcctagccttggtattcttccatgactttgggccaattatttaataattccaagcctgcatctttgttagaatctctctaagtttctcctgttgttatcctcagaacgggactgtgaggtacaggaggccacgtggtgtagtggtttaggtggacagacttcccagtgctgttctcatggagagcccaggcctgtgcctagctctgcagtgacagtgatagtgactggtgaaggtaaaggggtccacccagacgctcttgctgatggagagaggctggcctgtggctcttccctggggtggatattaacctgctaacgtgccgtatctagtcctgcttacattactaagtggtaggaaattttaggtaacatctcagactttaaagtggcctactgagcgggtagaaaagatagttaaatgcctagtatgtagttggtgctcagtaattgttgaaaagatagaagctttacttcaaagctcttgtagtttgttcagtttggaaaaaatattttaatgttgggatgtattaacagctggactggtggctgtctgaattgggaccatggttagggtgacgtgttttcttacttttttttttttttttgggtgagacagtcttgctctgttgcccaggctggagtgcagtggcagaatctcggcccactgcagcttctgcctcccgggttcaagtgattctcctgcctcagtctcccgggtagctgggactacaagctcgtgccaccatgcctagctaattttttttctatttttactagagatggggtttcaccattttagccaggatggtcttgatttcctgaactcgtgatccacccacctcagccacccaaagtgctgggattacaggcgtgagccaccgcatccagacacatgtttttttaaattaaatgtgatagataaaattaggctgcagggatggcctgatttgcaggtgcttcatgagcaagggtgcccagcatttatttgctgctactggagtctcatgtgtgcttgcagcctagccttaaaccctctgtgggcggtttggaatacttcctccaaatgacttttttggcagggtgggaagtggcgatacttcaggtgagagacagttgaaattaactttacacaagagccaaactgtaggctgacgtggggccactcacctttggactcacccctggcgggtcctgtaagaggtgctatatgcttccactttgccagctgttcgttggccctgttttgtcttctgctctttgcttgattagtgaaacttagaatggaaacaggcaagtttgatttgttataattcctaggagttctagcattaaggaagcagatgagtttgatttgttataattccggggactggataatttgctttctcctctctccatctttaattaatctcttaaaggaaaggaggacgacagcacttttcctgcagtcatcggtgtaggcttcagccttaactcatgacataggctggtgccactgaccacagggctgacctcagctcttggagcccatggggtgacaggacatcagcacctttgaggtggcggcatgaggcgtcttccaggccttgctcctagtgcttgaaaacactgctttagttttttgttaagagagacagaggccctacttactcctatcccacaggcatctggctgttgacctccttgttggcctcttaaggagagaatacttgagtattgaattagatcaacttttctgcctccagggaccctgtttctctctgctgagactggcagatggaacctggatttagtggatggtcaccaggtagcttgggcgacctgggccgctgggcccctgaggactcacatatcttttttcctgttcatgtgttccttccccacaccttggcccattctcagtccctcccatcctccttagctgaagcactcagggaggtgctgatggggccttgggacatgcactggagctgggtcccaccctcagacctgtgcccgactgtgccacattcattggaaagctgctttcacacttcctggatggaattctctcatttttttcagtgcagaatcactgagatgattttttttggataaatctttaaaggccagttgtaaatttattgatacttggaattgacaaaattcacttgtttagtgttgctaagtcatactgtgtaagtttgttgaacacagagttttcattttatactttcagaggagaaatgaaactgaatttcatggccagaaatatgtttgagtgtcatcctaatgtaagaacagaacagaaagcgtggtgacgtttcattgtaactctagtatgttttctcttttgtccattagactacatgaggaaataattgacttttataacttcatgtccccttgtcctgaagaagcagctatgagaagagaggtggtgaaacggatcgaaactgttgttaaagacctttggccgacggctgatgtgagtatgttctttggggttctgtgccgcaagtcatgtgcgagtaaatttaaacgccctgtggtgatgggtcggctggatccacacagaccttttcccatttgcccgagaaagcagttctccaagtgtgctttgagagggccaggtcgcctgcaatgctaggaatgcacggcccccggcccacctgcacctgatgccttcgacacctgcggtggccacgggtctgtgtgacactggcgcacactcaagtgcgaggaccattggctgagggattttttttttttttggagcggggagaatgatgtgattgtttctcttaagttcaccttaaatttagaaatttcaccctgtcacccacccctgcttccccaccaccacacatggtagcatataatgtgttcatttttgtaaaacttggaagtgttccctcatcacacaccactcttgcggtgaaggaacaagtgtttttgacatgtggagaggggccttctggaatgcttggtgcaggcggtgtgaagcctgcccctggctggctctgctcagcagccttccctgctgctggctgggctcaggggaccggcagggctggccgtgctctagctgtgaggggcatgtgtgctcttggtgggcggtgggcaaggacgacccagttttctgctcctctttaaaaatgatacatatttttggctttaaacttcagagccctaggacaaagccctgccccagtgccttagctgtgggtttaagaagaggttgaagggttcaaagctagctctggaaagtcctcagctttgaagggttgtagggtgaagacaaaacttgtttcacttcttaacttagagtttttaaacattcctttttggggcacgtctttaacttatagggaattttctggttaattgttacacagtcccctccatcagtttccaaggtagttttattttttacccaagggtatagtgagggctttcttggagtaagaagtaatggtagtgtggctgcttggtctgtggtatacattttaaagcaacctccctttctttcaggtacagatatttggcagctttagtacaggtctctatcttccaactaggtgagtaccagactgcatggcatgggctagtgtctgtgcttatttagaggctgggatggtgtctgggcgatattaagggctacaaatagattctttgtaattgagtctaaggggaaaaaggccagctgaaggaaaagactgtggttacagaaggaaattggcagaaagatttaatttagtgttatgaattcattgctttgcattttgcctcacacttaatttgttggggtgcaaaaatgctgggtgctggaaacgtgaagaagacaggtgggaggactgtgggaagtaaacgcaatagaaaacattctgctgatactttagaacatgtgtttgaaaaattgatcttatgttttaatgaagattcaagcaaaattctctttaaatagtattttctaagggtttttacctgataagaaaatgtcaaacaagtttgcattctaaaatgtaaactggtattttctcattaaggattcttgatatcaaaataaatttcagtgcactatctcattaaaagtttaccttctgttaacccactgctagttagcatttggaggctgaagaggctctaatctcaggatttgggggttgatgttagcagggccaatgggtaattgaacaggttcgggtatccaggaatccctgagtctgcaggagtgatccagtgtaggtagtgctggagtaggtgctgggagagcggggcctcagcctgctttgggggcaggcgttttcatctgaatcccctcacatacctagtgctgttgggagaatcatttaacctttgctttccatcttatggaagtcttcccttcagttcagcgaggatttgctgtagtttatgaactgcatttcacttccttaggggccatagcctagtgggattgtgtcttggcctttgggggagacgcaggcacatgtgcggtggtgcttatcctgcccacacgagtgtgtaggctgtggtaaggggagcagtggctgacgtgcattttcttctgtcagcgacatagacctggtggtcttcgggaaatgggagcgtcctcctttacagctgctggagcaagccctgcggaagcacaatgtggctgagccgtgttccatcaaagtccttgacaaggctacggtgagtgcctggctttggcccctctgactgggcaggagcctcgtcacatcccaggtggttacaggatacgcctgtgtcacgagctcgtggtattttacacagttattggccacggtttcgatgattaatctgcttctggtatagacaagtgttttatgtttttgttttatagtcatgatcaccaactgagaacgtgtttagtgagtctgaacttttgggatttgtgagcccattaaactgttcttggaatgaaaatatatgattgtgtctactcatgttaggatgaataggaaaggagagtcatatctgaaagcggacactgacgttcaggtgctgcccattttggagtgtttggctcaatgattaaaccatggtttttatgattaccatttgctacgttaaattcaagaggaacaacttagctgttccttcgttgttccaaaatatatacatatatgaaaagcctcttctctttgggggaagtgtggagatgagactgtgtctggtgtgtacttgttaaagtttatgtcagttcaagattctaagcccccagtgactgggaggtacttgcctgtcgtatgacagttcttagctcacctgtgcgactggctagcatttcatttttaaatttgtctgtcaacttatgtagtgtgtgattcacatcctcctagtgtttagcagagcgtcagttaaggcgggagcttcctcctgcctgtgcggtggtaggggagggggcattcacatgcttctcaggtgattccttgtcaggtgcttttgattgggactgggggcaaattttaaaacgtgatatatgcactgaaaagtgcacatgtaccaggtgcacctcttggtgaaagttaacgaagtacatgtgctcacgtcattgtcatgtggaccagagagagcaggcagcaccagaggcttcctccaggcccaggttccaaaaggggctgttggctcttctcatgagcatgagacgagccctggctggccactttctcaaatgcttatgggccttattgcacttagctctcccagccactctcaccagcagacactgttgctcttctcattttacaggtgaggaaacaggtgcagagaggtgaggtagtaagcccaaggtgggttggcggcagtgcgaggcagcacctgcgtttagcccaggccgctgttgtctgctgctcctctgtgtgtgccaaggggcagcggggaggtggatgggaatcctgaccaggcgaccacctttggagtagagaactaaggtgcggttgtcctgaggacacacagtaggttgagcagttgttgcagtaactgcttcgtcccagtgacctcattactgtcgcatattggctactaagtacctcttcctctgttgcctgaaggccagtgtagactgcagggagatcctggagcctgccactgctcctttccagggaccgtcctcaccatgcagccggtctgtcatgggcacaagagccccggggcgagggccgagtttaaggtgaaagcttgtctgtttggaaggacaacccaaaactgaatgttccagtgttactcctcacctcaggatttggggttgctgcggtgagtgagtgtgtgcaagagtagggcaggagagtgccaggagtgactgtggggaggagtccatgagatggaaaggaagctgcttcctgaactctgggtgtcggggaaggcatagccatcgagtgttgacttcttccaaagcagtttctgatgagttctttgggaaagtgttttgtttctctgccttcttagaatgtgctggcccccagagcttaccgtgtctggtggttgcagtggcgtccaggtgactctggccccactggcctgtcctcacacgctgagtccttggtggtttgccctcaagtgtagacattaaagcccagaataggtgtgtactgaatgccccgtccctgtgttgcctttcactggtgtttaaacctttcatcagaccctgattgttttggttaaaggaactccgtttttaatgcttcacggctgaaggaaaccaaacattgccttttttcctggaagaacacagtttcactggtggggtgggcggtggggcgtgagtgtggcctggacagttgctcaggcaggttttgagtgccatttggtgacagttcttgttggggagagtatttctaaaatagccctttatttgctgaatcttttaggggaaaggtttttttggtaaaattatcttaaagagtgaaaacccaaagtagataaacaataccagtattgtttggagaaacagtatcagtataacgtacaatttaaaaaatggctttgaagttacattaaattatttcaaaaattcgaccaaatagaagttgggggccaggcacctctccccatcccagcacgtagggtgggcgtggcagagactaactcatcatgttccctgttagctccctcccctcagtcctctttgatgcttggtcacctccggtgctgatccagggctgcaggggcgggacagaacgtgggccttgctgtgaggggctggggcctgggagttgtcctggattcagggactactgatgctgacaatgttctctgaccccctttcattactaagaaaaaacaccaaacctctgtacagctttggcagcattttgatgcctggctgtggagagtccagcatattaaagcagttcttaaaatgaaatctttacaggtaccaataataaagctcacagatcaggagactgaagtgaaagttgacatcagctttaacatggagacgggtgtccgggcagcggagttcatcaagaattacatgaaggtactgtgcttggtgacctcgcgcagcgaaagtgcaggactggagtgcttatgctcggcggcatttctcctacagatgtttacagctgtcagctgcacacaggggtctttcgtaatacagactacacttacattatttctcctacaatactatttctagagatactttgaaattacatagctgtttttaaaatttgcttttcttgagtaaccgttttatgaagttgacaactattttagagggctttgttaaaatgttgtttccagttattacaaagagactgcttctagctcagcgtgcctcattggcaggttttgcgcaggggtccagtggaagttgataaatcatgaggtgtcttagatatactcactttgggtggtctcagtgcctgaggatgggcagagagatttggttgagctgatgtatttgggactctgaccatgttcacgacccccgggtggtcgtgacttccttcctgttactgtagagactgtgcagtccttagcaggggcaaatcctgttacagtagaggccattgtagtccttagcaggggcaaatccacccctgcaatctggtcccgtcttgttccattttcaagggccttgcccttcaggttcccccttcccccttgtcatctggtctgaaggagcacgtctgcccccagcacaacacggacagcagcagccaggctttccctccctcctgtcatctcctgccgctctgccttccttgccaccctcactctgctctcctgctccggaggcccccactgtcctcacggcctgcgtagcaagcacataaacactccagcgaacgcactggtctctccttgagttcctttgtgcctgtggactcttcatggtgggggtgcacccctgctgagtcataaccgagggagagctccacagtctcgattttcacaagcccctcaggaaattaattcttagcgtctcccaaccagtttgagacttactgagagtcttcagatgggcaagaggatggaggaaacgttcctttctgtcttgtgtaatttgctatgtgaaatctctttgaaagtatggtaattactcaataaatctttttcttttggaatttacagaaatattcattgctgccttacttgattttagtattgaaacagttccttctgcagagggacctgaatgaagtttttacaggtggaattagctcatacagcctaattttaatggccattagctttctacaggtacgtatgctttcttgcgaccgtttctgttgagacatgtgtaagagtagacttttccaaccagttgtcctgtaggttccagcagcctttgctctccttttactatattgtttcaatttgttagaggctgatttctgattcttatattaaaaccctcttgatcaatgcacctttctggaggctcctttgtagctcattttgtactgggtgtaacggttagtgcggggtgcccatctggttctgtgggtccagtgcacatcagtgcaagctcaaggagttctctatattcagaaatgtccatttcatggtaaacaataaacatttgttggtgcttgcctgtgatttatattgaaaaaaaattgtctcagaataaagtttggtaccacatatgagaaaaggatttacaagattgctttctcaattgatgaaacctcattattttgtctgaaattatatgtggtccttattttgttgagtaacatggaaaatctatcagtagaaaacagatgtgttttaaaaaatattgttaaatgctgtgatgtattgataaactgtacttacactttttaaacatttaaaatcatctctaattgaaacagtattgtgtttatattttcttgagtgaagaattctgttctttacagaatctttcctgtataatttagctgcacaactggatttgtccatgtttaccattaatgttgtacttgtgattgtgctgagtgagccgtttctgttcatgatttggtaatgttctcactgtcgtgaatgctatgatagtagaaccactgggtaaccacctgtgatgatcgcagcttccttggtctgtctctgcagagatgctttaagagtagtgaaaatggaattccatgtctgttttcttacagttcaagtcacactgtcctgttttcactttcccctctgagatgtggccgttagtgtatagcctttcttccatagtttttggacattattttgaactaaacatgggcctatgttctttactaatatactcctgacttgcactattttcatggccttgtagtaacaacagctacttaatactttgatcatgcatgtgccatgtgtgcctgggcccagggcctcctgccaccccctcagggggtcctttcagtgatcctgtgaccccacaggaggccaccatagcatgagtcggattcctgggttcactgccacatccctgtgcctgtccagtgcccagagcttgtcctgcctcagcggtgtgactgtggcaacatttgagtttcttgactgagtgaggggaggaggcccaccctgccactggccaggctcttggatatgtgattttggctaaaagacaaggaataaggaagggaataaaggtgaggccagaattggaatcctcctcttgtgtggtggaaatacgcagagaactccgggattcttctgaaccttaaaaacatttgtatgctttcgagctgcagctcctcctggcactctgtgttataaatagtttcaagcaccgtgcttctctgagggatttctctcatgtgcccttgtccatccctttctagttggcactgtccgatagaattctctgtgatggtgacggccacttctgcatggtccggcaaggggcttggtgccacatgtggctactgagtattgcagtgtggctggtgagagcaacaagctggatttttaattaatttgttttagttcatttaaatagacatgtgggcaatgcaggtctggacagctgagaattatgaccgtcaggaggtgtggtggacagtggtttagttcagaacaagcccaatgcctgcttttgaagacgatatggcactgaactgagagtggtgctcatctgtgtgcaagcaggatggaagcttgctatagatattatgatgcaatactgatggcctatactcagctgaaatcagtcatttcactttctggttttatgtgataccgccatactgttcagtctaacactgacccctgttggttgtacttcagcataacaaaattaggatggtgagaatctgaaattacatctaccatccacacaactaagttatgcagaatatagtcaacttatttgccaatatttggttaatcaagtatctgtgtttgcaggaagtttgctgtatggattaacaattccaaaaattaaacgacaataaaattgagattagtacattgtattccttttgaacactcctcattgaagaggctgctgttcaggcttcctcgtggtgctggtgagtgagcgagtgcctctcactggtattgccttgaagaggctgctggacggcagacttcgttgcatcctattttctttagaacatgccctgaatccatacaaattgtggatgcattgcttttacagttgcatccaagaattgatgcccggagagctgatgaaaaccttggaatgcttcttgtagaattttttgaactctatgggagaaattttaattacttgaaaaccggtattagaatcaaagaaggaggtgcctatattgccaaagaggagatcatgaaagccatgaccagcgggtacagaccgtcgatgctgtgcattgaggaccccctgctgccaggtaagggctccccgacctccactgctgggagctaggccagcttcggggggtgggggggaggtgtgggggctatgttggggctctgaaagcctcgggcaattcatttgctcttgatgcaggtttctcttttttttttttttttttgagacagtctcgttctgtcgcccaggttggagtgcagtgacgtgatctcggctcactgcaagctccgcctcccgggttcacgccattctcctgcctcagcctcctgagtagctgggactacaggcacccaccaccactcccagctaattttttgtatttttagtagagatggggtttcaccatgttagccaggatggtctcgatctcctgaccttgtgatctacctgcctcggccttccagagtgctgggattacaggcgtgagccaccatgcccagccacaggtttctcttatactaaccagttaataatgacactttgagaaatccttatttaatgcttctaattaacttttgtctttccaactgtttacattctataataaagcagaattaaggaaaatctttttttttctgattatagaagtattccgtattcatcctaaatcatttgtttcccagaagtatgtaatatagaaaaacagtagatgccataatccctttatttggacatgtatttctccacttttttcgtatatggatattaatgtttattattgttatcttgttgaacacatcccacaacgcaaacctgttagtcacttggcttttgactttatttcctaaagatcatctcagggaaaacacattggctgatgtgttttgtttttgcttgtagatggcgtgatttatgcaggctcttgagtggtttttttggtagcacggtgtggcagtgtatcatccatctttttgtctgttggatcagtcacattggagacattgccattgtcttcaagtgtctactgaaatgtactcaaaaaaacaaagacccacataatcattgagatatataatataaacctagaaaagataaaattccggtacttagaatatatgtcctttaaaaaaaatctacacttaacctgattgtgaagaatgagttgtatgtagattaaaattcgaaacagtgcttttctgatgaaaagtaagctcctcttgactgacttccttggtgacttctgtgacagatttctttgatttgctgtcccagttttccacacatgagaattcacattccattctaaaggattcacttcctgcaggttccgttagtgttgactgagttggggtgcactttgggctagctgcccttctgactgccttgtgaggcctcctgattgtcagatcggctatcacactgggtcggtgctccggtctgcacaatggtagcttttggcttccttgtgtgtctgccacctggagagcgggctttttgtgagctgttgctccgtctgtgaatggcagctgccttcatgaggtgggccgtgagcacgtgggcaatgatgatgcagaccaggcccctcggcactcacagaccgtgccctgtgtatggtgattgacagggcctttgctagtgccaactgccatgcgtgcccattgtgttcgcctgtctgggctttgctggtgagggcctgcttcagagtcacttgtatgacaagatctgaaatgtggacgatggtgtgcgtgtggggagagtctgacatagcctttttgctgcagggaatgatgttggccggagctcctatggcgccatgcaggtgaagcaggtcttcgactacgcctacatagtgctcagccatgccgtgtcaccgctggccaggtcctatccaaacagagacgccgaaaggtaatgggttgcgtgtctgtgtctgggctcagcatgcctgtgggatggtagttacccctttcctgtgtcatttacctccatgaaatttatgaagggatgttctgtcgtatttcagtagaatttggatatgttggtgaaggaaggccttctaggaatgtgggatggctgtatgggattcatccatggttgagagttgaaaatttctttcttggagatttgacattttcttcagggtcttttgttttggagaggtgatttctagctttcaaaactttggaacatgatgctttttctccagccttgaaagcataaacattcactttctaagtgaatgtatctgatcacccaatccctaccatcttctcttatgtacactcgtcccttgttctacattttgggccatttttacagtcccaaaatgtagtcagatgtatttacttctgacccagatgatgctgtggtggtggtagtggtgggattggagggggtgggagatgagataggaatgggaaggaagagtaacgtggtcgtcaagagtggaatttgaaacagtttgataaatctgttccatggtggatgatggaataaacagatttcgaggcctggctcagcagctgctgcaggcgctggtggtgctggagctctgtgtgttcctgagccgctgtctgctcggtgttttcaggcggagctctgggccccgtgtagggcactcatctcagtaccatctccattctcgcctgtgcagtgggaggtgaaatgtcagcactgtgtgaccatcgagggtgggaaggccacgctcccctacttggagctgcctttcacagtggtcttctgtaggtagatgtactgcgatccagggtgtgctggagctgaatcattagaagtcatatatatttgtaaatgtattttagctcccttcctactgtccttcacctagtgagatgatctgttaggggtataaggtaactgctcaagaggggttctaactccctgatacctgttgtactctgttgatctccaacaatgtccctttgcagtactttaggaagaatcatcaaagtaactcaggaggtaattgactaccggaggtggatcaaagagaagtggggcagcaaagcccacccgtcgccggggatgggtgagagattaattcatttgtgttcatcctaaccactggctggcgtgttcatgcagaagtgtctctattcctttgtggtaaattggtcaaattaagaagatagctagtttttctgatgagcattaattaaaaacacaataagatctagagcagcactgtccagtagaaacaatgccacacatagaatttcaaatgcatgccacacatagaatttcaaagtttctagggccgtgtcaaatgtgaaaagaaacaggtgaaataattttggtagattttattcaactgaagtcaaaatgttaacatttcaacatgtaatcaacataaaaatgattgagatattttatagccattttttgtactaagtctttgaaatccagtgtgtatttatttgtacttacagtacatccttatatgggtgccaaattttcataaaaaatacttgatctatatttagattttagaaagttcacatttgaagatgatttgcatacccaagttgttacaagcatgtttaatgttttccaataactaattgactataatttttaaaattaagcaaaacctaatgttgggtttgtcagtcacattagcagcattcccggctcagcagaacccatgactgatgctgccggggcggctccacgccacagttctcaggagttattaaccaagactttttccctccacagacaacaggatcaagattaaagagcgaatagccacatgcaatggggagcagacgcagaaccgagagcccgagtctccctacggccagcgcttgactttgtcgctgtccagcccccagctcctgtcttcaggctcctcagcctcatctgtgtcttcactttctgggagtgacgttgtgagtgccctcccctcctccgtgtgtctgttggacagtttgtgtctctggtaaatgtccatagctgtgagcttaaaatctcccccttaggtttgctcaggttttgtttccttttatgtgtgtggagtgggtgggagggtagccccgtgatgtcggcaccaggcttcctttcccccactgtgaaccttctgaacctgtctggctcatgcggctgtcgagggcagtaatcctcttcacagatttaaaaaaaaattaaatccaagtatctcttctgtatttcctttaacattctgtttcagttttgatgaaattacttgaaggaagcctgggtagatttgggctgcccgttcagaagttagacttaattcaaataacctttcatagccagcctggaggcaggagtttcttttcatagcttgaaggaagtagtagtgcacctttgtggtacagctgtccttgttgttttttgtaccgggttcaaggattcagacacaccgccctgcacaacgcctagtgtttaccagttcagtctgcaagcgccagctcctctcatggccggcttacccaccgccttgccaatgcccagtggcaaacctcagcccaccacttccagaacactgatcatgacaaccaacagccaggtacgtggccctctggtgcccttcccggtggtggccccgggaagggcatctgagctgtgatatgcgctagagattcatggtcctttgaattcgaagagtaactttttgagtctttggccattgctgtcttattctaggaaatcctgtcttttttgtggtgttgaggcccaccatgtagagtttcagcagtgaggagactggttctcgagtgctgccgtggcttttcacgggggccaggtcgactgccttcctataagtttcctcactgccccagcatgagactgctgtcgaggatcatcttgagagagcgactcagtcgcgacccacttagccgggcaccaggcagcgccagacacttgtccctacttcctctcagaatctcagtgttgagacttttttaaaagttttaatgtgaacttattggacttttttcatgtttcaaattaggcatactttctaaggctttttctgttagaatgtactgtctgtttctaaaatttagataaaattagaaatctaaaggagaattttataaatactaaattttgtatctacttccgattatacatcactggaatatgtgtgggtataaaacccaacatgttaattgacttaaaaccattttctgaaatgtggggtgtaatttgagcataaaactatgtaggtatatgcaaaagtatcttgactcattacttggagttttgcagtatgctctggggaagacatgctcacaggatccaccctgattctggcagagcttctggaatgctggctctgtaatgacccacagagttgatgagcagagccatggcccagccagacaccataacgtgtctaattactgcataaatgtaaaattctgaggcaattatttacactcttaaaatgaattataccacagataaacttggttgcctttttatggtcatcacactggccctgacgtcctgggccatgtgtcacaaaggtgtttgttttaaccacccacaagccttggggcccttgagagcccagtgcggctgctgagctccagagccacactctgcggctgcttgtgtggttcgagcgtgaagtctagggacgctgagggtttctaggttcttatctaagaagactcttgtccacagtcgatctccagaggtggttggggtaaatgcatgggatgccaagatgcaacgaggtcagtgttgcaagtctgagaaaaggggttcttgttagtgtgcttctgctgctgacagtaatgggtgatgctgacgtagaagcagcccgggacctggacagcaggcaaggatggatctgccggccgtcccacggccccttgggccaccagtcaggccagtctcggtctctgaccccgagttcagtgtgtgagtcgtggattcatcacgggggtgacagttgttattctgatgatgctaatgttttgagtcatttgatcttcaatgagttttaaactttatgacttggattactgggcatactttatattctaattgctgtttcagaatatgaggtatttctaattcaaagcacaatattgttaggttatagtgaaacaaactgctttatggagcccacatgcaactgtgccatttatgagctgccctgtggcggtgctgagctttagaagccggatggttttcctgtgattgatttggtacccatggccgtctctgtcgttttcttcctagaccaggtttactatacctccaccgaccctaggggttgctcctgttccttgcagacaagctggtgtagaaggaactgcgtctttgaaggccgtccaccacatgtcttccccggccattccctcagcgtcccccaacccgctctcgagccctcatctgtatcataaggtatagctctttcctggtgcgttcacctgttcaagctgccatgtgagaggtggtgctgaatgttttctcctccaaagagaattccagagagatcatttgaaaacggaatttgctttcttgtcattcagcctcatgtttgcttgtctttccaaacaaaactttaaaaagttaaactattttaagatgtaagatacagtttaattggttgccacaaacatctcttaattcctctgttgaactgattagcataaaactaaaagttgaaataaggctcaaaatgaagacttttcttttcccatttatataattcatttatatgctaaatacctcggtttctaagaagcaaatgataaaaccaagagcagatcttgccatgatgtcccatgtatgctgctgtcattcccacgttgcctgatccccacctggggtaggagcaagcatcagggtgggcagagctgtgtgctgggcctcagcaggatcctggcatgcctgcccgtatggctcctcacaagtgcagctgtgtgcacggaaaaaacaggtcacattaggttcctatgttcttgaagtacctgaatgattgggagagccatgacgaggatcttccaggtcagcctctatcatgcgtgatgttcccttgggctgtgcggatgctcggtactttcatcggtgtccacacctcttactctactccactcctcctttgcttgtctaatcctactttgccaggaagttttattcttgaggcttgcgtctttggccctgtgttgtatgatggttgtttttaggagttaagtcatagaacatttcctcttggatttatgcatcccccatagacacattcagggtgaaagaacaacttcgcacagcagctccttcgttgcattttggctttgctttcccagcctcctcctgctgtttgtcctgctctgagacttccctaaagccggcgtgtgtttcctctcagtctgcttggccgggaccttgcagtgcggagaatgtgctttgggtgcagcccaagcacaggctgctgcatgccgggatcgacaggctgctgagggcgagagtaccaggccctggcatgtgtgactcgcttgtttctagaaggtcacagttgggggaagaacatgaccgggaccttcttatttctttttttttgggacagaatctcactccatcccccaggctggagtgcagtggtgtgatctcagctcactgcaacctccgcctcccgggttcaagcaattcttgtgcctcagcctctcgagtagctggaattataggtgtgtgccgccacacccagccaatttttgtatttttagtagagacggggtttcaccatgttggtcaggctggtctcgagctcctgacctcaagtgagctgcctgcttcagcctcccaaagtgctggaattacaggcgcaaggcactggcacctggccagggaccttcttatttctatggataagtagaacaagttagaagtgaggttctgctgaatttgtgtggtttgatcctggtatatggttgttgccttcagtcagtcacggaatgggaagaatacttttctgtcaaatggaagagttggaaagtcccagagggcaggtgtccatccctcctccctacgtaacatcacgtcggcgcttagtgtggtcactgccggaggacgtgggcattgtgcctgttgtctggctccaacattgctgtctctctctttctccagcagcacaacggcatgaaactgtccatgaagggctctcatggccacacccaaggcggcggctacagctctgtgggtagcggaggtgtgcggccccctgtgggcaacaggggacaccaccagtataaccgcaccggatggaggaggaaaaaacacacacacacgcgggacagtctgcccgtgagcctcagcagataatggctcctggctgcgtcggcctcccccacccctccgcagactgccccgcggcctcggccaccggcaggggaaccgagaccagcaccccgcacgtcagccgggctcacggcacgcccgccgctgatcactctgcatgtttctttgtgtggtggtcgcgtccatcttcaagaacagctcgttgtgctcatctgtgaagccttattaaacgtggacgttgttttctgccttcccaggattcttccttcagtgctgaggcaggtcaggctcaggaactgcagggacgtgaacatgcgcttgcggtttgcggtagccgtgtctgttccttcgcggtttgctattttcatttcctgtttgtcaaagcagcagaggagatcaaaccccgttcgtgtgtctttcctccatggataggcttgggaggtcattgttttactgccctcacattttgtttgaaatttcagaactgtttttctatgtaaatattgaaaacctatgatttgtgcaataactcagatattttttatttaatttcctattttcacataagttatatttaagggaggagggaattttttttaaacaagcttaggtcctttcccgagctgcattttctaagttgggtcatcgtgtcggctggttgtctgacgagcatcgttacaaacaccatgatgaggggtttggggttttattttgattcttttcttttggtcggagtgagtgaaggagtcaggtcgccctgacggttttccagagggctcggctccagagccacctgacggactgcccgtggccctgctgtcgggccccaggccgttgtcttgctctgaccacagagttttaatgttttggttttcagttcttttaaactggacaacaaatccagcatttcaagtgccagaagtataactttctaaggagagaagggttgtcacgttataaaatctttaggaaaatgtgaactggaaaatgtttcagtcagttttagtgacatagcctgtgatgatgggtctggtgactattattgcggaccgtggtacccagttttaggaatgtggagaaaggaattctgttgattccattgaggaatctgtagcgtatgcattcgttctgttaagagcaaatctaggagaagtgcttcagctgcccagtgcgccgtggggagtgttttaatggatcatgtcgcaggagagcacagcccagcgttggggccgggaccgctggcgcccgacgtcggaagcatacaggtatactatgcaagtgtattctgccacaacaaccactgtctttgttacctttttttgaacaagaatatatccatcctgcctaaccctgagtttttggagcaccacagttgtcctgggagttggttgcatcttgtaggccatctgacttcctgttgttaaaacaggggtctggtcttgctaaacactacaggtaggttggtctttgaagtccactagtggagaatgtcgagacaagatacttattaccatgacatctgatggatgtgcagcagtggggagttctagattgatctctgaatgtgatggacgcccagcaaggacaagctttaaaatgtctgcggtctgcccttttgaagcaggactggctcactctgtcattgggagctgtcggctgcgactgcaggttctctaggaggcattccagaatagagtagcacactgtgtctgcagttctcgatgaccgaaagttatcaaaaatatttaaaatatttaaattgtgaacctattgataaagaatatttataaaaactgatctgtaggcctgtactaatctctacgcattagcaatattgactgtaaacccacattaaggaaaccactacggggccggcagtgagtgtcccgtggggtgtgcattttaaagctcgattcatagacacaggtaccatgttccatttccgtcatggtgaagcagatgaattggcctggctaccactgtggttgtgtgctacaggtttgacaaaagatatcatgtttcgatttttttgtgtgtggacaacaatatggaagctaaaattgacatatttttatgtaaagtttttctattctttgatttttaataaactttggaaaccagttttgtgtttgtgttcaagtgtatgctttcaagggtaagagcggcttccacattttcagttttgaatttctaaattagggcaatacgttaaccagttattctaaataagattcaaaagaaggcagatgatcccagtcctataaagcaagttgcggcagggagattatgttgtcactggtccattaaagtaggaggaggtgaaaatacaaaaattagccgggcatggtgactcactgagtagctgtaatcccagctactcgggagggtgaggcatgagaaatgcttgaacccgggaggcagagattgcagtcaatcgagaccgtgctactgcactccagcctgggcgacacagtgagaccctttctccaaaacaaaaaacaaaaagtgggtggaggttctaggctttgcacaaggcctatataacagtaacagaaacgtgctctctgtcctcacattctgatgctaacaaattcagtaaaactgtttaaatggttcttacatgcagtttctggcaagctgctatgtgatattgtgtacttagactgcccaggggcaacattttactttcttcatttcttttaggtcaacattgcatcacttgcaaacaagacattacaaaccaaacaatcatagtgtagcttaaaatgcaacggacatttgggatttatttgagaactgacgtttgggatttatttgagacagagtctcggagtctcgctctgttgcccaggccggaatacagtggtgcaatctcggctcactgcaggctctgccttccaggttcaaatgatgcttctgcctcagcctgccgagtagctgggattagaggcacccaccaccatgcctggctgatttttgtatttttagtagagatggggtttcaccatgtcggccaggctggtcttgaactcctgacctcaggtgatctgcccacctcggcctcccaaagtgctgggattacaggtgtgagccaccatgcgctgccgacatttagaatttataaggaaaggtataagaattcttctaggccagtaagtgacagaactgcatgtcccagcctgtgcagctgtagaactatcaactgagtgcatgttgtagaactatcaactctagagttcttccagttgtcaaggtgggtgttgtgggtaaaacacgttccttccacccaaagaacagagtagccctgtcctcttggcacctggttcctgagggggtgcagaagctgctcacagcactgcctagtggtgctgtggggaagatggcaggcagctgggtgggactcgaaagcctctccagggggccgagccctgtgcagggtctgcatgagctgccatggtcagggtggggctggcaggagctgagctgggcctccgagggaactggagggcttgtagccaggggcatctggaggaggagcccacgacagtgctgaggtacagggcactggagtggggagatgtggctgagttagcctctgttggaggcgggactggtgggtgttgtgggggggcactctcggcctgccccggatttccccgtgggtggtgatgctgcctactgagccactggtgcctgtaggagagctgtctttaggaggcttccaggagcagccactggagcagccacgttaccagggagcctgaggccccacagaggcatctgaggtgtcagatttgggtcccgctctcctcggaagctgcgagtactggaaggtaagggctttctgggggcagagtgaacctgtccaggccactgcgtgaacatcagtaagcccaccacgtcatctcgggaccccgtggactggatttcatggtaacaggtaaccccctcatgaccggcttttgtacatgccttttgatttgtccggggtctcgagtgccacctctttaccccgtgagatacccagagatctctgtaaatgctgtgcccagcatgcctcggcctgggcatgaaccgtacgcaggggaggtggcgacatttgtaatcgagatatggtttcgttgtgactgggtcacctgtgatgccctaagccttgagaacttgtcatgtgtgtgtcattatcctcttgctgtctaacttgtgagattcttgccaagccttaggtgactttgactacctttggtccaaggtaatcgcctagaatggacggtggctcctgttaaacacacacacatcccagacaccacgccgggggctatgcctgttgtttaagaatggccccaaaatatgttgcctgcactgtgtctccagggcctgtgtgggaggggtagcttgggagggccgggtcccgctagccctgggttgcaagggtaaaccgcctggtgtcattcatggtgagatttacttaccccatgacttgatcctagagaaggccagctaaggacttcagaggtcttggagtgagttccaggcaagggcaagaaaccaaggcttttcatgggttcttaggtaaaggaagggggtgtggaccagtcgcactgggaggtcgagtcctgtctcctaggaagccacatggctgtggggtgagcttgcccagggttccttcctgccagtgtggatatggcgtcctctgcccaggcctcttgagatggaacgcttggagctcagccaatgctcaggtcgtcataggtgctgtggggtcactcacacttggttgagaggtatgtgactcgaggcagattcctgtcattcctgcttgtatttcatcaagtcctgagcgaggtttgtggtaatgagtgcaggtggctgtgaccgaatccaggtctcctcaacagataggagacctccgcacatcctgccatgtcactgttggaattttcaagttgggaggaatcttaagaatcagctagagcagctcagccccaaaaatgagaaactgaagtcctgtgatgggaagggcttctaaaggtcacgtggcacatccgccaggcccccgagagcgtcctgggcctggctcccacgctgctgctctttgcaaaggcctgtggcccctttttgttgtctgacgacatgtagagaaattgctgtgagtttacgcagaacactggtgcttacgctataattgagtctcaccaggttctgaagatgcgtcttgtgcccatggctcacatctgtgggagcctttcagggacagcggcatcaccagcctggccctgtgtgttgaaatggttatggtggaactacgtgggctcatgaaacgtgggaggcttccagttggactttttgtggccaatattgataggatcctgccagcggagagcccgggcctagggccaaacggctggagttaggagctggggtgcgcagagccctggaaggatttggcagggcaggggagaagggagtaacagcctcactgtgcttcaggcttttccttgctggttcaagtgtccgctgaaagcagctaacataatcagtacagcttctctgttttgggaaatcaaaccccaaatctttaatttgatttaaaaagctttccaggatcttgcctttgcctccctttccaatcttgccgctcggcagttatttctgctagaaaataggtatttccctctcagtgtctccaaatgtgaaagaaaaatcatcgtcatcaatagtcgggtataagagaggcagccttctccggaaggggaccagtgagcaggcagttcacagagcagaagggagggtggatctccagggaccgtcactggcttcaagtctcgcttctgttgcttcctggctgtgtgtccctgggcatgtcactcaactcctgtgctccagtttcctcagcctcgcaatgggccattgtacagactaaacgagtaaacgcacataaagcacctggagccgtgccaggcccgatgtgagcatttgggccgcatcgactcatgtgattagtcctggtaggcccggcgcagggaaccaagcctgtcaatttaaaggggagtggcaggaagaactcatttttaatgacaggcaaaaagggaaaatattgtaatacatggcttcctattttccttttccctttttcttgcctttgaatttataatgttgcttatttggtatttaagattcttgctctggagcttcaaacattttgaaagaaaaagtagtgtcatttaataaatgctgtcatttgacgctctggttactattttagggcagcatggtaggtagatactgctagttacttacccaatatccatttttctttattacttgcaggattgccaggtttagaaaatgaaaacgcaaaactccaagttaaatttgaatttaagtagacaatgaatacttttttggtggatgttccaatattgcatgggcaatttaactaggtgtcctgtattttatctggctcaactttgtttagacagcagggtgcacagctgaaaagataacttgtataacatccttgcagctaggggtggttgcaacagagtgctttcctttggaatgaaacttttctgggataaagtactttgcttccctccttggattctggctggaattcaagtgcaatgcctggtgacacagcagccatgttgtaatcatgaagcggctagctgtgtagtaaggctgacagagcaggaagctggaaaggacccagagtcgtgaggctttgggccttctttatatgttcatatataacatttgcatgaagaagacacccgcatctgtttatgccactgatattcagggctctcagctgaatacaattttaactgatatatacaggaaacgcacatttcaaacaaaatctcatgtgaaactccatatgggtggttctggttaaagcagtctggtagggaggaaaaaggggacatcgcttcattgcttgaatttcggctccactccctttcccttctcccagattttgggggatgccctgtggaacccctcactttctatggaacacatctttaagccaggaaaccacccattctttcccgctcttattctcatttttattttctcaactcttttgggcaggggttgaggagggtgtcctgtggtttgcatagatgcctgctggactgatcagagccctggatcccatacctggagtcgggagcttcccccagagccctgcaactcccaccctggttgctgtgcacggcggaaagggcccgtcaggaaacaggaagggagcctgctccactcaggtcatttgtcctaatccagctaaggcacaggctggagatctcaggggagtcccttccctgtgcccttaggaacgctggccccacctgggtgctggtttcaccactttgttaccaaagtctttgtcacagacaggtatcgggtgagcttacagctcagcctgcagacatagccccagggcagcatggctggactggttgtgcacagatgcctgctgcgggctgtggctcccctgcgggtggaagtcttggcccctggacctggtattcgaggtcttctgagacattctgccctcggaatgcttgcctccctattgatctgtcctgagtggcttgttactattttttaatagtcatgtatcagtagttatgtaaaatgtggcaaaaatgaatgactgaagaaaaatgagatggaaaaagaaaaaatctgaaaatatacaaaataaaagcccacactgttttgctattggattcaaagacctggaactatccttgcaggagcccctcggtgcccacccgtaggccacactcaccaactgcacccggggctgtgcttccaggacccaaaagctccctccttgagcggcacccaggcctgctcccactaacctcaccagccgcacccgtttctcccacggcccgcccaggggaccattgatcctttagtcctcttcggccagtgtacccccaatggcccaggacagcccctagtacacaatggctgtttcttgtggacaacgcgtggaggcacacatcagggccctgcttttaagaaggaagtgctgagacctgggtgccacctcttccaggagccatggatgccggggctcacctccagagactgaattaaacggtcaggagggagcctgcacaccggagagctctgagcaccgggcgagtctcccgtgtggccaggttgggagccatcgctgttcacgtggccagggctgccttgccagaggagctcatcttactttgccactgtgtaactgttcatgtttatcaagtcatgacctcccttgctacagtcactcttgtctttcgtgttctatttaatttttttaaaatgctggtggcctcactcacgaaatttttttcaccaccactcatggacaggacctgtcacgtgagacatgcaccaaagcacggatagggatggccacgatcctgcctggaacgggacttcagaactgggcagagcatggctccttccggtgggggccccagaactctttttcaaaggcaatggcaggacgggatccacagtgcttgaccctttgataaggagacatcctctggccaaaggtgacaccacagggataaggtccacgtgctgcttaacactgttttgggtgcagaacaaagggcagctgggacatttcttgtctggtctccccctctacgtgtcctgaatttctagcttgttcaaagtggtcaaagacccaaggctggtctggcacagaaggggtcgtgcctacatgtagggaggcactgaggcttctgtctcccgttgtatgtccccagggacaggacagccccctgcctgttgttccgagtgtcctctgtgaatggggaccatctgtcctccgggtaactccctcctccttcacctctgtccacccgctctggtagccaccccttccacgtgcagtgcggctctgcatcccggagagcctcgaggactccttttctcttctctagtgttggctcccatatctgtcagttaattacctattcattatatgaacatgtttctcttgggtcattatttaacttttcaaatttatcttctttactcaatatattgtgagcgtggatgtctgttccttatgggatcaccactgccaggcatgtagaaattgcttgatgtagtttcactggcttgtggagaggcagtcatgcttctctgtgcttctatgccaaagaaagtcagtagcaagcaaatagcagagctttagtgaataagaacaatattttaccttgaaaaaggcagcaggaaggagagttaagactcaagtttcctttattaaggaaaattgaaatctaggtacctacagatgctctttgacttatggttaggtcttagtaaacccattgtaagtcaaaaatgcgtttaatacacctaacctaccgaatgtctatcttacatgtgcttagagcacttatattagcatacagtcgggcaaaatcatctaacacaagccgattctattaatattaaaattgatgtaatatatgtacatattttagtacatatgatagtttaatatgttcatataatgtatacaggtcaaatcagagtaattgggagatccatcgccttaaatatttctttatgctaaaaacattggaattattctatctattttgaaatatacaatagactgttgtgaacttcagtgtccctactgatctagaactaccatgtgatccagaaatctcactgttgggtatatatccaaaaggaaaaattattgaagagatatctgcactccatgtttattgcagcactattgacaatagccaagatgtggaat

SEQ ID NO: 5 - DNA, 60510 bp, Mus musculus
  source 1..60510: mol_type = unassigned DNA
actttccgcggagtcggcagccgcctcgtgtgcctcggcggcgcttgagcggcaacagagtcctgcggcggcgcgcgcgtccgaggtgcccggaggcccaggtacgtgcgaccccagctaagctagcgcgggacggttgaccaggcggttgcggccccgctgcctgcagcctgccgcggcctctgtgacgcgcggtgcggcctcggggaacccgcggacggctggcagcagggacggggcggggcgagcgcggcggcggggcaggcggagcggagcgaggggcggagccggcggaggccccgccccggggccgagcagcacggacgctacggagcaggcgcgtcccgctgccgccgccgctgccgccgccgccgccgctgctgctgccgccgccgccaccaccgccgccgccgctcgcccttctcgggatccgccgccgccatttgcacgggaaccccggtgacaggggctcggcggaggggcggagggaggggggagggcctgcgagccccgagggcgggagcgacgccgccggcgccggccaggctccctgcgctaccgcgccgcccgtggcggaacccgggtggcagcggcggcggcggccgagggcgggcgtgcgcctgaggcagcggcggcggcggcggccctgcgggcggccgggaggggcgggggcagcggccgccgccgtttgatggatccgaggatcgcctggttccagccagagcagctcggaccgtccaatagtctgtggatgcagatctgggagacgacccaggggctgaggaacctctacttcaaccaccactgtcacagcagcggcgcgagcagcgcgagcggcgggagcggcagcggccccggcagccccggcggcacggccccggccccggccggcatgttccgctcgggggagcgcccactgggcggcctcgccgtgcccgcggagcagcgggacttcctgcccctggagacgaccaacaacaacaacaatcaccaccagccggcggcctgggcacggcgggcatcggcgggcccctcggcgtcgccggtcccatcggctccctcgtccccgcgaccggcggccgcactccccgcctccgagtctaccgacccggcctcgggcagcagcaacaagaggaaacgtgacaacaaggccagcacctacggactcaactacagcctgctgcagcccagcggagggcgcgcggccggaggcggacgggcggacggcggcgggggcgtgtacagcgggaccccgtggaagcgacggaactacaaccagggagtcgtggggtgagtgctggcgtcgtggcctgcgtcttggagggtcggcacacccgcacagcggggaacacgcccacaggcgggagggggggatgatgggtgggggatggggagccccagtggggcctccagagcatcggtggccattcattccttcatagcttgaccagtctcggtggacctttctttggaattcctttggaaaaagtggagctatcctctccttttcgtagtcatctctaccttcctcctctcctaactgtccacaccttcccgatgcattcttgcttgtccttctcctggatctttctatgctctagaattcagcctgccctctgatccctttctgtcctccacaccttgctccccaccaccacaccccaagatgaagccccttagcatcgggctagttaacttcctgagaacgtttctgccttagcgtctttagatgttaggaccaggaggtgctcttcttgccaagaatagaaacatccagaatgctccttcccctcccccagtccacgacgaaaatctcctcagccctgaaagacactgcctgctcttgatcctttggcccatcttttatatttacttaaggaaaaaaaaaaaaaaagccagtgtttgccatgtctgtcgtgagcagaaagcactgagtgacagctagcttgaagttgtcatagatggtgagcttttctgagccctgacagaactttgcaggagttcatggtcggcagcttctcactgtgaggttgtcactgtggcagaggtagcagtggttttcatttaggggtgctggtgttagtgtgtgaatgttgcaccaccggtgactttgtgtttaaagttcagctcttacaaaatggaatcttacctgagccctagtgaattatgtacataagctgggaatgtttccagcctgcttttcctttgggaagagcccctgtgttggctggattaggttactgagtcttttcttctcgttctttttttttcccttttcataaacttatgcttttggattttggtgtagtgtgtgtgtgagaagtgtagagtgtggtttgtgtagctccagtatggaaaaccagctctgaaggtttttgagggcagtttctcgttttcaggtactgatctgtagttaggcctcaggaaatcaatcagtaatcccgtgtgaactttcttagactcacccagtcagctctgccctggttttggcaaggagagatagttgtgtgtacctttttgaaggtttggtaaaatgagttggtgggttccatctgctgtagtgggctggtgtctgctcagtacactactattacaactcccaccttttatggaaaaatgcagtcatgtggttcaggagtgatgctggggaatgcctcatagctgccctcgttttccagaggagatccttcctggctcaggcatttttgccagcttcaagggcgctctccatggtcatcaccttacattaaagatttgtggttccatcctgttagccttcccatggcaaacataaacctgtggtttagtggcaacaacttaatgctgagggttctggtggtcttgtcttcctcgagtgagctctgcttaggtaatcactgtcaaataatacctggaaggtccctagctgcatttcactgatgcctctggtctgtttctagaaaagatatgctataaatccaaagtgctggctgccccttaatcctgaaaaagttcccacgcagaagcttcggatactgccttgccctcagaaggatcagggaggaatgttcacttccctgagggattactgcttaaaaactgattgtaaacttgctaagcggcttatcactccatcagacttgtattcttaccctgaagaagacggcagcagttaggagtggccctcctagtgtgagagatgcagtgaatagttcctaaggtttgttaagagactctgggttttcagtgattccattcctttccgatcttcgtggtccagtttcttagcttttaagggagtttagttattaaaattttcatttacttattgtgtgtgtgtgtatatgcatgtgttcacatgcatgtgtgcacatatgtgccaaagtaagcgagtggaagtaacaggacagcatattgggagttggcattcttcttcagggtcttgtagattgaactcagttcaccagtgcttgatggcaaacagctttttctgatgagccatcttgccagcctagttttgcttttttttgtttttgtttttgtttttgtttttgtttttgtttttaagagacagggtttctctgtgtagccctggctgtcctggaactcactgactggccttgaactcagaaatctgcctgcctctgcctccccaagagctgggattaaaggtgtgcgccaccaccgcctgacagttttgctattttagttacaggtatttaaacagtaggacttctgagtgtacctcagtatggtgctattcattatgtgacagaactttccctggggagacataacatacatgtctactcactttagatagggaactcaaaacagacttaagtgtgaataccataaaagccaaaacttggtgaaatctgactttttggggggttacttaaaggaatatgggtgaaaggttacttaaaggagccagaaatgactcagacatctgcatcaccaaagcctatccagctctcaaagctagggacctggagcacatggcacagaccgcaggcagctaaataggtagacagtgtcctttctaggtgcctcagtttgtctaaaactcctccaggttttttctgcttccaggtatccagctggtccctgagttgtcattgcagctgagctctgctcttctgagtgggtctctcagctttgtttgtttgtttgtttgtttgtttacttttgcatggagtggcacagtgaatctggttagtttcagggatatccaagctgttttgagttgtttacctccctgtttaaggagcttctttgtagaataaagatttcagtctcagaatacagcactgttcagaggtccagcactgttcggacattaaatgtagtgtgactcttagcagtatctcccacgtcctgaggtttaactggtaccctgctggtagaagaacaaattctcgtgcctgcattcttgagtggactctccaggtagggactcaggacatggcagtagaactctgtcctttatgcctttgggctagtggctgacaaactcctgagttttcattaagagtttgaagccaactcttaaaggtgtccaggctcttgagtcttttggtttcccctaacatgcttcatgttcttgcaagtgcaagatcctgtttaaccctctttatgtacgtatccccttgtattcactggatagacactctcagtagcccagtggggcatcacttgtatggtaccttaggtttcatctctagtaatcccacaaaaggggaaaaaggtggaagaggaggaggaggaggaagatgaagaagaaaggaaggaaggaaagaaacaaatatttggatttcatcctaaagtctctctctctctctctctccctctctctctcttttttaaatgaaattactctggatttagttactgtctctcaacctttgccccctgaagccgtaaacccaagtctagagagggtatttgcattgtctgttatttatttcagagattcaatccagggccttgtgtatgttataaaaggacctctgaccaatgggctattttctcttgtgctttttcctttttttactgtttgagacagagtctcagtaagttacccaggcttgaacatactctgtagcccaggtgggttttgatactatgatcttcctggatcagtctcctgaatggcaggaataacaccggacccaccaggcccagcttgcttgcttgcttgcttccttccttccttccttccttccttccttcctttatttatttatttatacaactgttttcatgtacccttttgctagggctggctgtgttgaaggatgctgtccgccagcactttcttgtccgtagaaatgagccagatgcccttttcttggaggtttagcagtatttgtggtagagaaggaaggatacatagagctacaagcatgggaacttagtgaatcggagccctcactcctgacctggttttgagaaacttctgtggaaggtgcctgatactttccaacctcctgaggatgagacactgtggctgccctgagaggtctctgtttgtcaaggctaaatgactttaacaatatagatatcaaagagacttgggaaataacctcacttttggatatttgggttgctctaggaattggacctgggctcccacatttgtgctagagagtgaagctgtaccccagccccctgaaaacacaagtttaagtaattggtttttgagctaaggaggtaagaatgagtggaaggaagggcaggttaggaaaggatcctggctgagtggtgggcttagtgactacagctttgaaggtcaggggctaagaggctgtgctctcctgcagaggggccgcccattctccattgtagtgctctcctttggctgtcagtgctggtggctttctttagtgctgctgttgtgacatgtttctttagcctaaccacgcatgggtttgctgaccctgctggatacctgctcagcccagcagatgagcacaggttggtaggaaagaagttagaagactttactctgcctttggtgctcactttaatttctgtgtgtttgaacatgcagcttactgtaattcttcagtgaaccgaactcttccggctcttttgttttaccgattgggatactgaaatgtattttcaagaagacatcatattgtggattctatgctacctgtaaattaatagttgagaattgctgaattaattccaactgagattcgttccttctgagacctcatgtgagagttagtttgactggtgggtgagtggtgtttcttttggagtcattggcacaggtaggcctttacaattcctgttatggctctagctgttgagatgcaaagggaagcatcacccagagcattttagaatctgccctgtgcttacacgggagattaggacaccagaaggaagaaaggaaggaaaaggaagatgtgagacgggtgagagagaggtcaggatcctggtgcactgggtggccctcatctcagctgctttcaggaaatagctacctgccctagtgttgggcccagaaaggttcttggtaggaatgttatgggtttcttggtgcactggactgggcagatagatggcagtcttccttgatgagttctttctcagacaatctgagaattgtagaaatgaggtagacatgagagcagtgaatagtaggcattcttgtttgtttttgtttttgtttttttttgagacagggtttctctgtatagccctggctgtcctggaactcactctgtagacaaggctggcctcgaactcagaaatccacctgcctctgcctcccgagtgctgggattaaaggcgtgtgccaccacgcccggcatagtaggcattcttatacctgccgaagctgaggaaaatcatgggcaccttccctgtgggacacagagacgctgctcttgtctgacttcacacagctctctctcctgctctctgatgatagtgtttagtggcggacactagtccaggatcacacccacaaacactagactaaatcacatacacacggagtgccgtgttctcaatgggtaatgggccgggaaagctgctgcttttgaagactctttgttggtccactgattagcatatatgcatgtttctgcagagctgactggacggctcggagcctcctcaggagctgagaggccagtagactgctctttcagtttttgctaaggactcagcacctgtggaacatcaaaaaggcttcacatttgtcatactgtatatatttgttacacataatgacttttttttaaatgattgtatattgaatttgaagatggctttaagctaggctttttattaatttacaatcacctttaaattctgagaaatttcttgatgatataagtaagactcgaatactaagaactttggtcctaaaaagttgaatgtggggccgtttatgatgtctattgcccagagattacacattagtatttaatgacaaaacataggaattctcgcttaggcccttcctgaaatagtggtttgtaattagtgcagaagtgcttgctttcctatcagcttagtaagtagaagtgcgcatgaaaatagaaaccgttattaccacattccaaaaaaaaaaaaaacctgtcgtacttgttaccataatcaacctcttcatatgtagttatgtttcttctttcttttgagataatcgaaaggttagtaggacattatagaaattagtgattgatcattgtagtcttacttcactgtcgtaacatctcaatgactagtttaattcagaaccaggcaactaatattttcataatttgtaacattttgtattttgtcaggtttacaagcaaatgtgtgttgagtacagctttatgacatttgtaaattcatacacaccaccacaatgaactttcagaactaggttaagttcatcttggatactcattatgtttccctcatggggaaccacaaaaaaaaaaaaaagatacaactattgtattaacttggagatctctgatgcccgtagttagacacatccttcttggcactaaaccttaacaagcactagtctgtcctcctctctgtagctttgcccttttgagaatgtgatgtaaatggaatcctgtcttctgtaactttggctgttttcctgttgctgaaatgtctcggcgagtcattcagcttgtcttatgtgtcagtagttgttctgttacccagtagtgttccattgtatggaggcactagttgaagaatattccagaagagtgctttccagtttttgactgttacaaataaagctgctataaacatttatgtacatatttttatgtgaacataaggcttcattttgggggaataaatgcccaaaagcaccatatggtaagtacatgtttaaagaagtcgctaaccagtttttatacttccttcttttgtgtgtgtacatgtggtataatcacatgcatttgcaggtcagaggttcacaccacgtcttcctttttgctctctaccttatttttgaaacatgatttctctgaacctggagtccactgttttggttagactgattggccatttggcccccaggatccacctgtcttcgtcaatctccggcatcacattgtgattacagacaggcactgccaccatccaccttgtcttcaggagtcatatctaaactcaggtcttcatgcttgcatagcaatcactgtactcactaggacatcttcctagactccttccactttctacacttgattttaggaggctgagaatcaaaactaagccattttctacagtggctgcatcatgccaagttcctgtatgaaggggtccagaatctatctctccagtacttgccgtggccaatgtactgtagtacagctcttttcttctaataggtgataccaggcagctcctgtgctgtggtcataccagactgattggagtaaccctgcctaataagtcatgcccttactttccatctcaatcatatctgggattgcagctcggtggtagaatcttttttttaatcctatatgcacaaagtccctgttttctagccttgcattgaaaaaaaaatctgaattcccaaggcttatgtttttctggggaaagggagtgtgttcttgaaatactcctctttctcccctcagcggtgtcttactatatttgtctaccggattggtcttgaactagggctggtggtctctgcacctcagattcctgactagctggaacttcagttgtatgccacaatactacccttagtggttgtggtgtcttgtcttgtaataaataaatgtaatagcaagagaaagaaaaataaattaagtagatagagtctcactatgtagctcactgtgtcttgaatactggcaatcctcctgcctcagcatgccgaatgttggaggacttgccagtgatggctaccgtgcctgcttgtagttgttgagtggaatgaaacatccactgtttgtctcccagttgtcctataataacctcttaaaaacagcttgttcaaagcatgatccaaatatggttctttccctgctatttaaatcttttgatctataggtttctctgccatctgtttccctctctattaaatttgttgaagaaattaggttacttttttctttgttttagttgagatttttggatgtggtgtgtgtgtgtgtgtgtgtgtgtgtgtgtgtgtgtgtgtgtgtgtgtgtatgtgtttagacatgtgtgcctgtgtgtgcacatgcggcagccagagaggacacgtgtggtggtgccctgctcttttgctttgcatttaatccctttgagatgggcgctctctctgtgtgtttttggttagtctggctggcggacaaactctgggatctgtctgtcttttcccctgtcagcacaggggttgcagacatgcatgtagggtcacatgtggctttcacatgtgtgctaggggtctgagttcagattctcatgcttaggcagtaactgagccatctcctcagctctctgtaaacttccctgctctctcctttctcgaatcatggctctattacgtcactttactttgtctctttggttagatattaaattcataattggaactagagactgttctgatattttttttcatgaatattcttaccaatggattatacatatttttaaaaggaaaatagccagagggatggctcagtggttaagagcattggttgctcttctagagacctaggtataattcccagaacccacatggtttataattggtggtaactccagtcctagtggggatccaacaccagctgagtgtggtgttatgtgccttagatccactgtggatggaggcaggcagtggatctctctgagttcaagaccagtctggtttatacatgtcctgatttcaggaaagctagggctatgttgagagaccctgtctcaataaactacaacaaccacttaaaggtcaaatctgaatagcttttgtgatgtaggtagccattaatgatgtttgtctgatggtattaatgcactaggattttaacctcatatttggcttggatccttccccttgttagttggtacttctaagagaaggtctccttccttggctccctctggtcatgaggactcagttgtctctctattggagtagagcttgagtgaggattggggtgggaagacttacaggcacgagggagaagtaagggccaccgtgggaaggggaaatgggctttgggtagcttggggtgtgtacactcgtgtgcatgagtctgtgtgcatctatgtgtgtgtgtgtctttctgtctgtctgtgcgaaaagagaaggaagttctcacaatgaattgtactaggcctttggtctctggttagaaattgcatgagagccctttgcccattgccaccctgtcttggcttttgatctgtggcagccaagggaagccatatgagatctgtgacctgttagcctctgctcaggagacaaagttcactcagccgtaattcagtattcaggaacagtattttttggttttgttttgtttttcctttcagattagccttagattatttttccttgtctgtttttttttttttttttttttttttttttttaaagatttatttatttattatatgtaagtacactgtagctgtcttcagacactccagaagagggagtcagatcttgttacggatggttgttagccaccatgtggttgctgggatttgaactctggaccttcggaagagcagtcgggtgctctaacccactgagccatctcaccagccctccttgtctgtttttaatattggagccaatcattcttgttttcaggctgatagagataaggttgtaaggtgactccttccccctcagacactttaaagggaaccacttataacagtagagcaagatcattgagggcatggtggcctggagagcaagactgtgaggactctggaccctagccctaggtcagctttagctttctcaggagttctttgtggaggcagcagtcagttgtgggtaagatggctgtcacaccctctttttttggttttctgagacagggtttctctgtgtagccctggctgtcctggaactcactctgtaggccaggctggccttgaactctgcccggtggctgtcacacacagtctaaataaagagggtcttaagatgggcagtggtggtgcacgcctttaatcctagcacttggggagcagaggcaggcagattactaagttcgaggcctgcctggtctacagagtgagttccagaacagccagggctacacagagaaaccctgtctggaaaaaaagaaaaaaagaaaaaaagaaaaaaaagttctcttcctttttgtgtcctcctcaaagtatatgaacttggttgagttttgacaagcagtttaggtttatggaaaagtgagtgggcagttagtccagtttccatgtgtcctctgcgcccagtttcccatactaagatcttggtgtacggtgatgaacttgatgggcctgatgccgttaagttcctggagctcaggaaacatgtgacaagatggttctgttgaccacccaattctcttttatccacctagtcaccccccccccccctttttcttcctgttcctgaacacctggcaaacacctgtcctttctttgtctcttccgtttgccataccctgggctgtattctaagtcgccatcactaagtcttttcctgttacctttcgctcggcagcctgcttttgagatcctttatgtattttcctggcttggtttagctgatttatgtagtgctaagtaatactgcatggtatagatatgttggtctgtttatccactcacctactggagaatagagtgttgcttccaagtttggcattggtgcacaaagctgctgtaaatatccacgtgtgagttcttgtgtggccatacttctagcactttgaataaacaccagggaacacaactgctgcattatatggtaagaatactgccaagagggtcttccaaagtggctgtgtcattttttgcattcataaaacagaaaatagggtttcttgttatttcacttcttcccctgggttattgttttggctttttgccattctagtatatgaatagtgatagcaatttaatttgtggttctgatttccttcatgtgctggaagtatctgaatgcatcaatgttgagtgttggggcggctgagacaggaaggtggtgcattcaaagccagtctgggcttcatggcaagacactgccttttagggcttttagtggccaaacctaggatctcatacatgttaggggtgcacacttgtcactgagatacatccaagcccaaggccctgcctcaagatgaagtaaattagtaataaatgttaggttatgagagctctgccctcatgactagattgatattattcttgttctttttctccctttctccattttcatatgtattgtatgcatgcatatgtatgtgtgcctatgatgtgtgtgtgtgcatgcacatgtatacacatgtatatggaggcctgaggctgatgtggagactcaccctcagttgctcttccattactggtgcaggatctctcaagcccctagcttgctgatgtgcctatagtatcactagccagcttgcttggggggatcccttgcctctggcgtctctgtaattataggcaggccactatgcctttttctttttttttttaagttttgtttattattatacataagtacacagtagctgtcttcagacactccagaagagggtctcagatctcattacatatggttgtgagccaccatgtggttgctgggatttgaactcaggaccttcggaaggtgcttttaaccgctgagccatctctccagccccacttccttcctttttaaagaattatttatatattatgtgagtatactgttgctgtcttcagacacacaggaagagggcatcagatcctattacaggtagttgtgagccatcatgtcgttgctgcgagttgaactcaggatctctggaagagcagtcagtgctgttaaaccaccaaggcattttccagcccttctcttactttcttacattgctgccttgtgtgtcggtgttgacatttctctttcatatgcttcccaccatgtcgtgcctgtcatgagggagcagaaaggctcacaagtgagggatagttattttcttccttgtttttccctttggtgaattaagatctgtttttgctgtgtagcccaggctggccttgaacttgagttctgcttcagcttgtctagcactggggttacagacatggctaaatcatctttatccttccagctgctagaactatgggccaaataaagttttgtccattgtaagctacctggtctgataaagtctattgtagttgtataaacacagacaaagagcaccagtgtagcaaaaagctacacctccacccccatacaattactaaagtatctgttctgatagtttgcgtattatttatttgggatatttttcttgttgaaatttaagacttcttggtatgttttatttgagatatttttcttgttgaattttaagacttcttagtatgttttagaatcagcctatcagataaggacttgcagatactttcttgcagtctgtggcttacattatcccatctccccacccccgaaaagtttaatttcagagtagtcaaataattataatctttcagagtttgtgcttttggtaatatgtctaccaaaaccaagaacacttaccctttttcttaatatttagaagttgctccatttattgattgtttagagacaagtcttgctatttagctcaggatagtccaggttagccttgaagccacagtcttatctcaaggttgtaggtatgagacattgattgtttttctttcctttcctttcctttcctttcctttcctttcctttcctttcctttcctttccttttctttatttatttttttggtttttcgagacagggtttctctgtgtagccctggctatcctggaactcattttgtagaccaggttggccttgaactcagaaatccgcctgcctctgcctcccaagtgctgggattaaaggtgtgcgccaccattgcctggccaattgtttgctaagctggagtcttactgcattgtccaggttgctctctatatcctcaagtgacccgttcccttctcttcctgagcccccggcaatactgcccacttcactgtctccacaattttgtttttccagaaagtcacatatgatgatgtagtgtcttggtttggcctcacttagcaggagctttttaagattctaccatgtcttttttgtttttttgttttttccttctattcttttttttttttttcttcctttccctgcctctgcctcctaagtgctgggattaaaggcatgccccaccacggactggtacttccatgacttgatagtcttccttccatttagaatgactggcatgtgccaccagacccatatttttttggagttttataattttgtgttttacattttagtttgtggcccattttaagtgtgtgtgtgtgtgtgtgtgtgtgtgtggtatagtatctgtgtctagaattgtttcatttgtttaggtttatttatttaatttatatgtgtgagtgtttagtctaaatgtctgtatgccaaccatgtgtgtgagaagtcaagagagggtgcagattccctggtactggagtcatggatacttgtgagtttccgtgtggctgctgtgaatcaaacctagggcctaaggttaataagggctctaaattgccgagctatctcttcagcctctagaatctgtgccccgccccccacctttgtatagctctgattgtttgggaattctgtctgtagaccaggctggccttgaactcacagatatctctacctgcttctgcctctcaaataatgggattaaaagcgtgcaccactgttgtgggaaatatttaagaagttaacccaccatctctctgctccactggtccgtgctcctgctccagtaccggtactggcctgccacaacactatgtcctggcgcgtcccactttggcctgttctcaccgctgagctactctctcccagctagcgggttgatcccgctttcatgcaacaccctacacaccccatgatcagccatctcaacacctagttacccagtagaaaactgacacttaaagcttaataatccaatcagatgtatataacaataagatcacaagttacaagatgccaatacaataatttcagagccaactgataaggataaagctttaccccaattattctaatctttgtgacaatcttagctacttgtggctgttcaaaaccatgtgggatcaggatcatcttcctgtttgtctgcctccatgttggcttctcccctcctcctacctctctctccctgtcccccaaactcttagctccaactcccctttcctgaagtagcaatgaaataatgttatggctcggggtcactccagcacgaggaactgtactaaaggtcacaccgcattaggagggctgggaaccactgggatccttgtttctttcattaaagtttcagagtctccttcatatagggcctttgcatattttgttagagttcagagttagattgtttgtttgtacgtgtgctagtataaatgatattatgcttcaagtttcaaattctagacttatttaataccttgtatctttaatcccaagtgctgggcttaaagcatgtgccaccactgccctgctctggccttgttcttaatcggagtgggtgggaaaacatctcccttctcgctgttggctttttgaagattgttttgaaaatcaggttgaactttccttttctaattgctgaggttttttgtgaaagtagttaaaagtttaacccttttttgcctattaatatgattatagaatttttcttctctggttgcagtaatggatattatttttgaactttgaactaatcttgcatatctggagcaagttcattcggttatgtttatttattaatcatcctgcttcagcctctttaagtgctgatataccagctgtgcaccaccaagcccagcgtcttatttaagtcttggtagttatatcttcatgcattggtatgtctcatttaggttaacagatctgagggatattgttcttcatgacacttattaatggctgtaagatcagtagtgattatttgcctttcattttttgtttgtttatttatttatttttgatttttcgagacagggtttctctgtatatccctggctgttctggaactcactttgtagaccaggctggcctcgaactcagaaatctgcctgcctctgcctcccaagtgctgggattaaaggcttgcgccaccaccgcccagtttgcttacttatttataagttgtatgtttttagccaggtggtgacccatgcctttaataatcccagcttttgggaggcagaggcaggcagatccctaaaattcgaggccagattggtccaaggaatggcttccagggcagccaaagctacataggaaaatcctgtctcaaaatccaaacaaataaacaaaaaaattgtatgtatgagtgtgtacttctatattggctttgtgaatatgggtgtagtgtctgcagaagccagaagagagcattctgttcccaggagctgcgggtactggggactgaatgctcagaatcgctgagctgtctctccagcctcctttcactgctagtactagtaacttaatatttgtatgtgtatttgcctgggagtatgtttgtgcactagataggtgcttggccagaaggcatcaggtctcctggaactggagtcacaggtggttgtgagccaccatgtaggtactgggagcagaaccgggttcctctgcaaaagcaactggtgtgcccttaaccacttataattctccagccctgatattggcagtttttatatgttcttcactaatctggctacaagttgaccagtttttggttttttttgttgtttttttaagaatttatttatttattttatatatatgatgagtatgctgtagctgtcttcagacacaccagaagagggcatcagatcccattacacatggctgtgagacaccatgaggttgctggaaaacagtcagtgctcttaactgctgagccatctcttcaacaccctgttagtttttttgtttttttgttttttaaataaaagtatacatatacattttaaattttatatttattttatgttttcattttttctgcctgtaattctgtgccccattcccattcagtgtttgcaggtgccaaaggaaggtgtcagatcttttggaactggagttaagggtggtggtgagctaccacataggtgcagggaaatgaacccttggtcctttgcaagaacagccagtacatgtaacctgtgaaccatctctctagcctattaaggttttatagctgaaaatatgcccttctccctttaaaaaaaaaaaagtgctggatatatcagtttctctttttttgtctgtgtgacttcaaagtgccaaatatgtaaaacaaccagaggccagctatggtggcacaggcctttaatcccagcacttgggaggcagaggcaggtaaatctcttgagttcgaggacagcctggtctacagagtgagttccaggacagttagggctctgttacccagagaacccggtcttgaaaaagcaaagcaaagcaaaccaaaaaaaaaaaaaaaaaaaaaaaaaaaatccctaaaccaacaaaccaaagaacaacaacaggaaagaactaatgagaatgctatatccatggtccattgatggtgaagtgataaacaacatggtgtagctctacacagtaggctgctgtttactgttaagaggagaaattctgctgtgtgaaaagcaggcacagaggacaagtattatacagtgttgcatattataggaaccacctaggcagtggaacatacagaggcagaaatcatgatggttattgccaaaggctgggggaaagaggaaatcgggtaccatgaaccctaacagtttcaactgagaatgttaaaaacttgaggagagccggggagtggtggcgcatgcctttaatcccagcacttgggagacagaggcaggcagatttctgagtttgaggacagcctggtctaaaaaatgagttccaggacagccagggctatacagagaaacgctgtctcaaaaaacaaacaaacaaacaaaaaaaccaaaacaaaacaaaaaaaaaaacttcaggagaacaggtagtggcaatagttaaacactaaggtgagtgaatgtactaatgacatagattgtgtaattaaaagtggctgaaagcacactactacttggtgagcatacctagggtcctgggtttgagccctatattgggaggaaaacaatagttgtaatgttaaattatgtattatatatattttaccataatttgtgaaaatgagaaaaaaatttaaaattattttattgtgtcttatgtgtgtcttatttgtgttggcacccatcatgttacagaaaggtgcgagccagctggtgtgagttttgggaacagaccttgggttctctgtcctgggtgcttataactgcagagccacttctccagcctggatgatttatttattttttaaaaaatctgtgtggtattgataatggaaagggtttaaaggaagctatttgaaatgtttaattttcatggtgggaggtgactggggatattttgagcctctgctactgagctacatttccagctttcaaaattttaacttaatgctagtattagcattatttaattttcatttgttactgttttgatagaattgttatatatagttaggatgttttatgatgagtaaatcagcacaaaatcgtttatttgtgttgagtcttgtaagggtctaagaatcgctgaaagaagaccccagactcaatagtattcaaagacaaagagtgttctgtagaaacagccagcatgagtgggtggaggatgggtgggggactgaagggggtggtcaaccatccaagcaaaatggcaaccatgggggaggggtctcacagacccattttaaagccagttacggattttccagttgtggttgggtcatcttcaatcaggattggttgagcttatggtatgggatatttgtacacttctgattggttcctacctggagggagagagggttaccttatagggactatttctgtatctgttataagcccctggccagatgtcagagcagttgctgattggttgctctttttcttcgttttttccaagaagcctgggatgtcctgggaaattgaggcttaaggcctaacatggctgcctattattctaaaatggagtgagttaggtcctttcagtttgatactgtagaacatatggatgccttcactgctctgcttatttgtagccagattctgagatttgggttttgagattgaaggatcttcaggggtcagtggagcatagctgtgtttagagtgtttgctcaccattcaaaggtccctatgtttgatccaacaactcttcaaatcaacagaacaccaaaataaattacttaagcctcagagattctacctggtttttccttactaagtgcagaagctgatactgcccttccctggagctgagcccgtttgcccagcatgctgggtccctgtgaacacctgaatctggtcctgtcaacccaggcccgtgataacataaaacctggggcctctgtccatgcctgctgcctcttttcacagctttgcaggtaacagagcatgcgtggtgggcttgacacttctttatttattttatgtatatgagtacactgtagctgtgtagatggttgtgagccttcatgtggttgttgggaattgaatttaggacctctgcttgttctggccaaccctgctcgcatcagaagagggcgtccgatttcattatgggtggttgtgagccaccatgtagttgctggaatttgaactcaggaccttcagaagagcagtcagtgttgagccatctcgacagctcttcttcatgccttttaagtcctgattggtaactgggattgaggactggaaccttctcctgggggactgacctaacaggtggtgtagtgtgtggtggttttgacaccatatttataaatgatcatgcggcacaaattcagaactaccattcaaggatctattgacgggatcccagagctggccggttgtcagaacacatggaccattagatgcctcagactcattgctgtgaccagagctagtgtggaagtggggggctgagaattccactcaggatgcagtatctcttcctcactactcactcttctgaattaaggccaggccaagtgaggtcaaccttaaaacttaacatgggccgggtgtggtggcacattcctttaatcccagcactcgggaggcagaggcaggtggatttctgagttcgaggccagtctggtctacaaagtaagttccaggacagccagggaaacagagaaaccctgtcttgaaaaaccaaaaaaaaaaaaaaaaaaaaaccttaacatgagcaagtggattggtttaggccttaacaccatgctttttaaagttaaaagtgaaaatgcttattaaggaatctgaggttatattgcaaggtttctcttattaaactttttggtctttaggcaggccagcttgtgacaggcagaggtaggcagagttttgaggccagactggtctacaaagcacgtcctaagccagccagaactatatagtaagactttgcctttgaaaaaacagaaaagacgagaaattggtcttgcagtgaaagtgttgggtctttttgtttccttttggctgggtttacttttaagtgagacagtgagttagaggaagagggtgtacctgtttacatgtgaacattgtgtgccgggagctccttactaagttttgaattttccttaatggaatcttagataaattacctatactttttgatctggaatttttttcttaaagtttatctttgtaacaacttaaaacaggaaaaagaggggttagagtcgtacataactaccattctgagttctgaccttgtcttgggaggtgtaattgtttctagtgttctgaggagtcttgcaaacctgccaggtaaactggacaggaaccaaagaagtcattatttagtaatttatatgtggtatttttaaacttattcacatgtaacctctcttagtctccccctcgccctctttgtggaagtcatgtgaatgttctgtagacttcagttgtagggctgtgcagttgttcctgctatggagattgaacacttgctgctctctctttttttttttttttgagacagggtttctctgtgtagtcctggctgtcctggaactcactctgtagaccagactagcctcaaactcagaaatccgcctgtttctgcctcccaagtgctgagattaaaggcctgcgccaccacgcccggcacacttgctgctcttgtagaagacctgagtcctgagtttggttcccagtacctatgtagggtgactcacaaccacatgtaactctagctccagggggatccaatatctctggcctcatcaggcacctgcatatataacccctatgcattaatgttgttttgttttgttttgttttgagatagcacatctgcttgtctcttagttcactatagagaatgacactggctacgtaggatgccaagtaatttaaaagttatgagctgtgtattcctggaatttcctgtgtagttttgaaactgtagttgagcatggataactgaaaccactaaaaggaaaacggaggtaaggggtaggagtgggcggggcctgctttacagtgctctgctgagccactggcccagggatgagtgctgaagtgctttctgtgtttcctaacctgctgctgctgcgagcattctctgtgcctgctggagtctctctgcttgtagaacagagctgagcagttcactgtccaacaggatctgcctaaggatctggagctggccctgttgctgagatgataagaggtaaagcgacactcaggagattgctggaaccgggcagcggtggcgcacacctttactcctagcacttgggaggcggatttctgagtttgagtccagcctggtctacagagtgagttccaggacagccaggccagggctacacaaagaaaccctgtctagaaaaaaaaaaaaggagattgctggatctcactgacactgctccacaactcctgggaaggtggacagggcagggccggtctgctgggcgccactatagaaatatttgttaaaatgctaggatggtgatatgaaggtggttggcagatgttggtggttggactgattgtgtcaaaacatcaagcatcaagagaaggcttaaaaaatctaatatctaaagtctttgtcttctactacttgaatacagcatgcttcaagcatcccttacagttggacatctaaattgtttcctttttgtctgttggaaatgaagctctagaaacatgcccagcattggctttgcagctattgtcttcgtctgcgttgcttagaatacgcttcttaaatgttgccagggatcttgtgaggcccagcagtgagggagggagcctggcactcagcttggagcctcttctccacccatgaagagtagagatccactcttttgtttgttgggatggtgcttgtcaaatttttgcctttcagactcttggggtgtgtgtttcacaatggaaatgagttacttgctgttgactatggagtttgactagtgtgttaggtttttaggggaagaactgggggtacttcgcatcaccaaagtggaaaggtgtttgtccttggctataagatcctgcacgtaggacttaggtagggtggatacaggtcggtgctagagtgcttgcttattactgtatcccaggaacttgggcttggtcctcagtgatgttgagcgaacaagcagattaatgggaacttgttagttcagcagcagctctgtctgccagtgagtgatctgtcataaaatgaagcggggctcctcggtcagggtcagtgtaagtgcgtgccgtaggtgctttttggtgaagaagttaaggtaggaaggggctttgagtttgtgaggattattgaatattatcctgtgaaaacagcagaccaggagagagagagagcttagctgggtacacgccacaggttgaaaactgctgatgtagaatgactcaggagagttttatttttacatttcattgatttgtctttcctgttttcctttccattttgtttattgtgttctacatttttttctaaggacagttttaattccttttgtgggcttgatagctagtcatatttttaagatttgttttactttatgtatatggatgctttgcctgaatgtatataaatgtactatgtctgtgcctgatgtttttagagaccagagacatctttaactggatttacaagtgtgggccatcatttgggtgctgggacaggatctcagtctatacagtgacagccagtgctcttaaccagtgagacatctcatcaatcatttttaaattattcttcttttaactgtgtgtgttttagtgtctttgtttatctgatgaatgtatggagatgagaaaacaggttctgaactcagttctttttactttttttttttttttttgagacaagtgtctcacaatgtagctctggctaccctggaatttgctatgtagaccaggccttgattcacaggcatccttctgcctctgctgcccacgtgctgggattacaggtgtaaaccacacatcagacctcatttacaattttacttgtgttatttcaagtatgtggtaaggaatacagagtaacatcgaatctttgagcatctacccctgaatttaagaaactctaattctattttaaacttattttctccttcccttccctccttaaggacaacagcttttcagatgtattacacactacgtggtgcccctcctgctttccaggtgattgttgccacaggcaggacaggaccttccctgttcttcccaagtaatgcgccatagtctgtacttacatccatgagcaatgtgcactgtattctgtacttacaaacattgtattagtgggttatctttcttactttagaaaactatacgtgtgagcattcatatatacatatggcacagctctgtagttcagaagataacttggaatttgtttatgtgcaccaacacttcagtgtctttgtgttttaggagagcttaatttgagaaacatggctgcagaatagaaatttaaaacccatgtattgccaggcctggtggaatgtgcatggagctcgaggtgcttacaagaccatttgcaggttgtgtcaggatgctaacatttgccacctgttttcaagccatgtaacctctttctagtggacaggtgtcattatgtccacttgatagatgaagaaaatgaggctgaaggaagtatagggaccggtatataaatgtgtgtcagaagcaggtatagaatctataaggcaaggctccttttgcagtgatcaagggactatagtcagtgatcttggtgcttccaggaagcctgtggtagagtaggaccaccgaagtctcagcgagcacttacgtgggtgctggggagtctgaactggttctcatgcttgcatggatagcatgggctttacctactgagctttagcttgttaaatatgtcatccagtggcattcagcacgtttacatcattatgcaagcctttgtttttgttttttaacttttttatttattttatgtgtgtgagtgttctgcctgtgcgccatgtgtttgcattgcctgcagagggactggagttacagatggttatgagggaattgaacctgggttctctgggaaagcagtcaagggctgttaggctgtttagtgctatagattgaacccttgaatgcaaatatcctaccaaagccctgctattatccattcctttttaagcagttaaatatttgggttgtttgcttattttgtcttttgtggccctggttgttctggaactcactttgaccaggctggcctcaaattcacagacatccacctgcctctgtcccccaagtgctgggatttacgaatctctgctttcagctcctttatatttatatctgggagggaaattgttaggccagatggcaactctgtgtttaagtttgaaatgacattttatattttttattatttttaggtagggcctttgttgcctaggaccttgagttcccaagcctactggatggattgctagaagagacttaaagacttaaagatttgtgttagacttcagaagccagagccagggctagtcgtgtaagcctgtgaatttgatccctggggcctgtgtgaggttggaaggaggacaaccatagagttgtcctctcacctccacttaggtgatgtggcacaggttggacatgggattgcataagtgtgtgcatgtacacatgtgcattcatgcacacatacactcacacacacagaataaaaaattgtttttaaagataagcatctagaaacatgactaagccacaggttggtggttcgccatatgctttccactgaactgggagtgttagcagcttggttggtcagcattgtgaacggcagcaggcagcagcagcaggagcttcttacaggtcttagacagcctcagctcctgggacccctgccctgaagcggcagcccttgagagtgacacatgtcacagtcctctgacctaggtgccaggatctccatcctttgagagaagctagtgttgcgtgtcttaggcctgtaggagcgctccctggagagagaagcttctgctgcgtctagagcccaggcctacattgtcttcagctcctcacagccctcattgtttctgctgcctcttttgtttctgccctgacaccctagctgcccttgtgtatttattgtcactgtctctacaatggctgcttccaccctctcttttttatggcagcagcctagccagctctgctaattatatagttagaagcaaagaaaaaggcacaagaatccttttattggtcccagtgttgcagtaccagcccaggagcccctctccagcgtggttaacagagagctgagcagccttcagtgtgaccagaaatggaccagaaatggacatctctatctcagaatttcatgaaaaagaacaactgtctgaaccaaaaaaatggcacagtcccttatgcaaatgaagaactctccttacactaattgtaggctttctgtttccccttgtttgggtgaaaatgcctgcaaggtaaactgtcatctggacctcactgagtctttcatggtaaaatgtctagggcaatgcgtttcacacaggccagccccctgctttctggagctgcagttactactctcgggtagcaagcagcagtggcagctgaggaacttcatctgatacctcagtgcagtattcacagccagacctatgtgaggatagtcctgcagctgatggatggcagcctgggtgccagggagagctctgatagctgtcagttaggcaggtggcccttagaaggatgcttatagaaaagacaagccttcctgctgcagccttcatatcatagtgggtttgttttctgttttgtttgttttgttttaatgttttttaaaatggggaatgtgggagggggatgtatgctgcatgtatggagggaggtcagaggacaacttgtggaagttacttctctattttcactatgtggatcttagggattggacacaggcagcaagtacctttaccctctgagccacccaatagcaactctgtttgttttaatgtagttatcctcttagatgtaaattgatgtctgattgtggttttcattagttaaactgtggtggtttgaataggtatgtctcccatagacttctgtgtttgaatgcttggcactattgagagatgtggccttactggagtaggtgtgggcttatgggagggagtttgtcactgtgggggcaggctttgagatctcctatgctcaatctctagccggtatggaattcagtctccttgctagctaaggatttagatgtagaactctcagttatctcatctctgtgctgccatggactgaacctctgaacatgtaagccagcctcagataaatgctgtcttttataagagttgccttggtcatggtgtctctgcacagaaataaagccctatctaatataatggctaacagtgcatttttatgtgtctattgcaatctgtctttcttctttggagaaatgtctgtttagacctttcttttttcccttttgcattgttgaatcatgcttcttacataatctgtcaggatagtatttctttatcaaatatataacttgcaaatatactctcccattctggggattttctttttactttcagttttttaaaaattggctttaaaaatttttatgtgtatgagtgttttcctgcatgcatgtctgtgcaccatgcctggtgcccttggtggtcagacgagagcattcggtatcctgaaactggagttacagatgttgtgagccagcatgtgggtgctaggaattgaaccctgagtactctggaagaccagcaggctctcctaaacatgagccatctctccagcccagtcctttactctcttaatactttactcttttgcacaaaggtttacaatttttgattaaattcaattggttcttccttttatatgtgtgtttagtgtcatgtcttaagaagtcagtgccaaatgtagtgtttcctacagtgtgctttccagagtttgaggttttaagtgtggctttttggcactctgagtgaattattgaatattgttgaaggtaagtgtctggccttattcttttgtatgcggatatccagttccctcagcaccatttgttgcatgatctattgaacagtctttgctcactagtttgagttatttttattatgttaactaagagtgcacagaaatagaaacttcttgtgttcaaggatgatttaaaaggaaacaaatttacgagttagagatgctattgaaatcgtataagatgttaattttcatggcatctcatgataccactcgaagtgaacattggtggtgttgtgtgatgtggaagtgtatttaggtcctggccagagtgacacccttcggttatgaaaatttggcccttgcagcagtgtcacctgctcttacttggcatgcatgcatgcatgggtcaccaagttgtgctttgctcttggctgttaatacagaccaggtaaatctcttgcgtggcctgagccggacaagcaggtgcagcagtatgtatgtgctgggacacagggcagccctgtgctcccaaggaaggctagcccttagcccaaacagaaaccatgtgagacttcctgtggcaccgtccactttctcttgtgctcctgaagggttaattccccccacccctgagagttagccgggaagagggaattatttacacctcgtctgacaaagggagcttgttttgcttttcccagaggaattatttacaggatccttgcttctgtgctccttctgtggttggttagggtgcttcctcatggctagtgaccccacctttgcctcagttccactgtaaccacagcttttcattgactgcaactagattcaaagcttgtgctcccagtcttagatgggattaggatgtttgctcttaacactgattttactgccaagattctgtcaagttgaagattcactacatcagttttgtttgcatgaccatagagttcttcacagctaaattcttaactgggttgtccctccctgtttaaagtgctagactacccatttgatttttaactattgaaaatgagtgatgctgagataagcttccgggtgaggacctcatgcccctggggcagagaggttctttagctgttttctgcttggatacagtttggtttagatattcagtttggttttggttgtttggttgtttgttttgagacagggtttctctgtgtcatcctggctgtcctggaactcactctgtagaccaggctggcctcaaactcagagagatctcctgcctgtgcctcctgagtgctgacattaaaggtgtgtgccaccaccacctggctagaagatggagtctttttatgctgttctgagcaaagagtctccactttgtaggctgtgaaggcaagtggctgaaactggaagtgaagctctgcctagctgctggaaggagtggctctccccccccccccccccaggcggaagtgttccttgtcttgtatgcatgtcaggaagtgtagtaatcttgtatgacttcaggatgggattcatttagtatgatagtatacaacatttttctaagtttgtatcaagtcatagctaagcagattatatatatatatgtgtgtgtgtgtgtgtgtgtgtgtgtgtgtgtgtgtatatttttttttttaaataaccaaaacaagagtgttatgtgaattaacacattatgggctaggtatggtgccacacccaccagtgagagtttgaccatgctctagtgaatatatagacaatacaaactgggctttttccctttgatttttacttattattttaatctatctatccatctgtctatctgtttattttgatgggggggaataacaagggcaaggtggtcatggaaagactgggaagtgagtgtgatgggttgcataatgtgagtcccagaggatcactgaaaatactacatggaaaaaaaaagagaatttcaacatatgaaccagggaaatggcctaggatggagagagctgagtggcagagcacttgtctgtgtgcaaggcttcgggtgtgaccccagcactgcaaataaataataacaaaagggaaagggttcactgatccagcaatgtaggtagatatcaaaatattgacactgaaagactaaatgagtaaccctacaagtattttctgcataatttgctttatatgctttttataaaattatatataactatacgagatgcaaaactaaagtagtaaaaaggacatattaattgttgctgacgatgggtaaaaataaaagcaggctgaattatagagacccaagaaagcttgggaaggtgtcagtgtgttcactattacgattataaagatagtctgacatccactggccaaaatcaagttttaatgtgtagcttattattatacttaagtgttatggagctaaaataataccggtaaaaacctcagcttcactgggtggtggtacacacctttaatcccaacactcaagaggcagaggcagggaggcagagaggcagaggcagaatctgtgagttcaaggccagcctgatctgcacagagaattctagtacagccagggccaaataatacagaataagacaagtccttattctcatcgatgtgtatcctcattagctgggtaatagcacacgagctgatagttctggggacttgggcccaggcctcacatgctaagaaagagcactatcactaagctacatgtctattcagtgttttgagatagggtcttgctgcatagtttagacaggcctagaactcatgatctttctgtctcagcttcctgagtgctagaattacaaacatgcacaccactttggctgatagagtagtttattctaaaatgcttttaaaattggaaccatgtaaaacaatcaattttagtaatataatctcttgttcatttttaaagttttaattatgagggatttattttctttttaagattgattttattttacgtgtgtgttttgcctgtgtcctgtggagctcagtagtggttgactactggagctggagttaaggatgggtgtgaagtacatgtgggtcctgggaacacaacctgggtcctctgcaagagcagcaagtgctcttaactgctggatcacttttattagtttaaaaaaaatgtgtatattatttttctgcatacatatctgtgcacctcttgcatgtctggtgcttgcagaaaccagaatagagcatctaatctcctggagtgaactacagttggaagccaatatgttggttcggagacttgaactgggccccctggaagagcagcaagttttttcttaaccagtgagtgagccatttctccaaccctaatgtgattctcttcattagtttatctatttacttatttgaggaaaagcctcactatgtagctctagctggcctggagctggccttgaactcacaaagatctgcttcctgaacgttgggattaaagttatgctccagcataccttgactctcaagtacaccatagtttatggcattaagtatagtcacaatgtattgtggcaattgtcactattaagttccagaacattcatcaccaaataggaattctgtttctttccagcccctggcaaccaagaggctctggattttcttattcttcatgtgtcatgtaatagactcatactgtatggggctggttttatagcttcaggggtttagtgcatggttctcatgctgggacacatggtgggatgtaggcagacagggtgctgaaggtaagtttagaattctacatctagattagtaagctgtaggaagacatgcacgcacacatgcatgcacactgagcctgatttgagcatttgaaaccccaaagcccatctccaagtgacatacttcctccaacaaagccacacctcctaattcctgtcaagtagtgccactccctaaagaccaagcatccacataaacgaacctgtagtggccactcctcttcaaaccaccgtgtgtgtgtgcgtgcgtgcgtgcgtgcgtgcgtgcgtgcgtgcgtgcgtgcgtgcgtgcgtgtgtgtgtgtgtgtgtgtgtaacttaatatcaaccatttgaaagcatacaatttatgttgttgtcattgcccgtctccagaaatttttatcctccccaactgaagctgaaaatactgctatattttctcttccatccttgcccctatagccatgattgtctattatgtcaggattattcctgggatttgacttctctagagacctcataaaaatgggatcaaacagtatccttttgagtccagctgatttcactctgcgtaacagtattctcaaggtccactcactttttatccaccattcctcgcccatagactccttcacttggctctgcctcaggctagcatgaataacactgcagtgaacaggtctcttctggagacctgctctcagttctttgggggatgcacccatgtgataattctgtgtgtgtttgttagtttttctttaacaaattgccatgattctctccccatgctgtccattttatattcccggaagcagagacaagggtcctccagctgtgtcacgtgcttgtctgcacacgacaacgtgtttccttttgtttgttttatagaaatcaccctaatagctactataaaattttcatacactttacattttaatgtataaccaagtcttaatgtactgctggacagcagttacccctaaacccttgtggaagtgtggaggactctgacaactgtcagtctcctcctcagccttacttactcccaaatatcaaggcggcccagggaagtacacagtgtaccttggtctggcatgtaacttttgaagactttatggaacctacagatggcagaggtcagcaggtgttccctgtagagacaatacgtactccttattgggtctgtggtctctgtctcagccgctgcactctggtgtgctggtgacaagcagctggagtcaacgtggaaaggagtgaggggtgttgattgtattctagaatagttattcatatgtgcttgaatttgaattatatatactttttaaaaatttattttgctattttatgtgtatgagtgttttgcctccatgtatgtgcgtgtgccacactcatgcttggtgccggaggaggtggtgagcctctatgtgggtgctgggaaccgagccctcctttgcaacaacagtactcttaagcactgagccaactctctagcccattatatatgcttatcgtgtgtcgcagtataattctgacttcttttcaaccatttaaaaatgaaactataaaagcacttttaacttgtggagtgtatacaaatggcctttgttttgctctttgggcatgaactctgttaagagagagctgagtagatgtcgggggaggggggacgaatgtggtttgttagaacttttggaaattccattttctggttaaggcaaagggtacagtacttagctgatggcaagaagtgctatgctcagagtagaagaccctagaattacagatggggtaggtgtgtctataacaacttacatgaagagaccaggacctggcacagtgctcagcatgttgtctactgttaagtgttttatgggacataggtgggaggagtgtctataagaacaagaaggatggttagtggctaccattgctaaatgattattttattggtggacctgtctcagagacacatggtcattgctctgtgcctgagagagtccatgaagagctgagagagctgaggtctgggagtcatatcaggacttggacgcatatggagggacaggcagtaggtgaagcctcctagcttgttacacacacacatggactgaaaatgaaggaaggtgccatcttcctgtttctgctgtaatgaaacaccagagacagttttttttttttttttgaacagaggttgcttttatttagctgaagctgagaagcccaaagtcaaagggcccatgccagattcagagctgcttgctgcacatgtcttcctagcccctcactagaattgttcttaccccatgcctccaaactctgccagcccctgcccaatacctgcctccagatgtttcagcacctcggatagttatagcagcaaccccactctgagtgccaaatcggtagtagaattttgacagaagcagaaacaatagggatgtagacagacagaccagtgatttattaggaaaatcagcttgtgtggttatggagggacagaaatccctaagcaggctgtctagaagtggaaggtgctgggacgccagtactctgtaactcttgtttctgtccagaggtctcagagttagggagccagcaggtggtgtttctgtcagtttgagaccaaaggccttacagattcagggatgggctgctataaattctggagttcaaaggccagagaacctaggcttctgatggccagagaggcctcatagttgtgtcgctatgaggaaatctctagatgtatttctctttgattctctctgttaatgaaatggactggtttccataaaagttcttaaaattagaaattaacttttttagaatctatctgatgttaatagcattatggatacttggaccagagattaagtaagagcctatggacacaataaaagtccctcttcaaagctagaggcacggctttgcacagcacaggaaggaatatggaagatggccattctccatgtgactctccaagccagcaccagaattacaggttttattcttatgtgagaacttcagatgctctgtgagtttgttgtctctagtgactgtattaagcagaacagaagctaaagatggatggagagaagtcagaatgatatcatcaaaagggaccaattgcagagcccaaatatagacccctaagctgctctgaatgaatgatgggtgttggctttaagttaggtcaaccctagtcacaggcttttttttttttttatgatagggtttcatgtcattcaggctggctttgaactcactgttgttgctgaaggtgaccttaaactcctggtaaccccacctgcaattcttgaattgtaggcatgcgttgcaggaaatattaaaaaaggaacaggcacattcctgtgcttgtgtcggcaactctctgccctggtggcatggctggccatgcactggtgacctgtttttatcaagtggaggctctaactcacaactccaagatctctccacccaacttgctaggttccctctggcaggtcgctaccacgccaatcccatgattcaaatcccccacaccgctgtggtgcacctcaggcagacccacactttgctgccatacctttctctcttgaacctggaggagacgggagggaaagcatcacataaacttagttcacgatagtcgcaagtaagtgttagtttttaaaaaaaacagggctggagagatgatggctcagaggttaagagcactgactgctcttccagaggtcctgagttcaaatcccagcaaccacatggtagctcacaaccatccgtaacgagatctgactccctcatctggagtctctgaagacagcaacagtgtacttacatataataaataaataaatcttttaaaaaaaaagttgaagccgggcgtggtggtgcacgcctttaatcccagcactccggaggcagaggcaggtggatttctgagtttgaggccagcctggtctacagagtgagttccaggacagccagggctaaacagagaaaccctgtctcgaaaaaaaaaaaaagttgaaaaaatatagcttaatgttttttgtttttgtttttgtttttttcgagacagggtttctctgtgtagctctgtagatcaggctggccatgaactcagaaatccggctgcctctgcctcccaagtgctgggattaataaaggcatgagccaccactgcccggcaaatttttttctgtagaacttatatttagctttacaaagtttatagcagaattgagtggaaggtacagggattccctgtatagccccggcactcccttgagtccccttatatgacgttctgcccagagcagtatagttgttcagttagttaatcttcattggcttgttgttagcgagtggtccctgatgtgtattggtgttgtagtaaatagttaatctcagttgatcattcagttgatcattcaacttgatcattcagttcccagagaaaagacacacaacctttttatttatttatttatttatttatttatttatttatttttaaagatttatttattatgtgtaagtacactgtagctgtcttgagacactccagaagagggagtcagatctcgttacggatggttgtgagccaccatggttgctgggatttgaactcgggacctttggaagagcagtcgggtgctcttacccactgagccatctcactagcccccaacctttatatttataataagccttaatcagctctagagctgggcagttatctaccctctatggctattatgtctactactctatcaataactacgagttataacttgccatgttgcctctggacagcttttaactccagttggccagccctcatggccatgtttttatttctcacccatcgagtcttctctccaccttctccctctcctagaagtccttgcctccagccccagcccaaatcaaactcccacttctctgtcttctgtccagctataggctgtaggcatctttattcacctatggggataacttgggggtcaaggttacatagcattacttgggtcaacccaagaatctgctccctgggacaactagggctgtatttagcattacaacacatagaacagactaaacatcaacatatcagtattgctggaacttcttacttagtggaatgatggatccaagatgcatcacatagaattggttttattgccccaaaattctttgctctgtctcccaactcttggcaaccactgagccttcttcccccatcttcagtttgactttccccaagatgtcttacagttggaatcacacagtttacagcctttgaatttggcttctttcactcagcagccagcatttaaggccctttaaatttgtcctggcttggtagctctcctcgttctcatgctgtgtgatcttcctttgtacagatgcactagaccacatttcctctgctgaaggccatgttgggtgcttccatctgggcagccatgaaaggcctgttctatatgtcctggtgtaagcttttgagagcatgtttttagcttcctggattgctggattataaactttgagaatgtttgtttgtctattgattgattggcagacaggttctcactgtgtagtcctagcacggaactcaaagagatgtgctggcctctgttgggattagaggcatacaccaccacttctggcctctagcttatgttttcattctcacttaaattttttttcttccccaaaacctttaaaacacacaaacaaacaaaaagatagaaagtatgtggaagctggggccatggcttagtggttaagagcactcgcagttttatttccagcacccacgtgttggctcctaactgtcttatgccctctctggcctctgagtacacagacatgcaggcaaaataccgtacacataaaatgtaataattaataatgacaagaacaacacagattgtgagtggtgaccccgtgactgggatcagacctgcacacacattaaattatgcctgtattgaattgtcttgtaaaattaattatctgataatggtgcttatttcccgagggtttgctttgtgcttggtaaacagtaaaccttgtagagatacagatagaagctgcctgtatctgcactcctgctctccccactcagcaagtgggaaggaggctgtctgtgtaaaatgcttcccccagcacaggctggctgtgtaaagctcaggttttgtttttgtttttgttttgtttttgcttgtttgtttgtttattgctactttaaggaagaaagggaacctgggacatgtggcccagggtgcttggaccggagtgcctctgcgtgtgtgcctttggttggttttgagcagaaagaatttggtaagctatcagatcagcatgagaagaaaggtggacctgggaagcagagagcagaggaggaagtcctggtgaccgttgggactgggagtaagatgatctttggttctggtcttgtggcatttgaggtgacagcctggcaagaggtctctggcattgagctggaatgctgaactggctaactatgtttctgtttctaccatgaattctggaaccttctggagccacttacattgctttgtggttttgtagggcttgattgctcacttcccttagtgtcttggtatagttgagaaaaattaagtggtatagaaagttgtaagtgcctggccttccctggaggaaaataccaccagctgttatagctactttggaagtttgtctcaaatttgaatacgtaattctgacaccagcagacactccttgaatgtgtatgaaaccgaccatgacttgttagtgctctgtaccaaagtgctgacactgagtcctgggcttggggtcctccttgctccctggtggccgtgctgccccacatttgaccacagaatcccttctacagttgctcaggctccctagttacgcaggtagaacctttgctctacagggtaagattattaaaataattttctctagttgatatatttccttgttacaaagatttagtttttaaaaaaatacatgccattaacatacattttataaaatgggtcagtaaaagtatacatttcaaaacagtaaatccctctctttcatgtcactgcttaaacttaatcactgctgatagtaagttccagatcttgctttgtgggaacagggaaatgtacagctatccaagccaagcgcattcatgtgtacatagcatgtatcacgtgggacaccaactgtggagaagacgactcttaggaccggtatgtcactaagcagactgtaatcctcactggaagggtagacaacagattcttgccggtgatggaaatccttctgggttctgttgttgactgtaagtttttagcatttcctgatcatgagttctgtgctgtgcatccttgtacccatgcctttatattaatccttatggaatatttctgaaggatagggttagggagtttggaaattacatgaacctcttgagatatttggtaacttatacttccacctgtagcatctatgcttttttcatcatccttttcaaaaagtgtccgctcttagctacactaagacttaactaagactagttactaatagttcttgggtgaatatagtcagtgcttttgtgtagcattagatccttgtgttaaaaagcctggatggtggttgtaacagaccttgttttacctttgtgtttgtgtttgcatattccatgcccttataagagaattgtgattctttgcctactttaacttttagaaggaaagtactattatccaaatggaggacacttgggtacaacaatcaaatccagcttctcttgttctctcaaaacacagaccactgttgtgtccaagtgtgtaggggtttccagtctgtacctgggaagctaccaattctgtagcagacgccagctgggtgtctatcgttctagttctgtagtcccctggtctagttcatgctaaccttacttacccagggatagcaacagagcttttatcactggtgtccccagcccaattgcaagccccaggttgttggggtagtatgcaacacttttactagattatcttagaaaagctgccttcgaggaataaatggcagagatgcagaaacacaggatggtctggaggctccccagcttctgtgccagtggagttgagatgtgcccccgcctggctcttgctcccccgcagggaagcgccagcatgttctgtcagtaagtaagctctgctagaccaggccttttgggcaggtgtgggaagctttttcctgtaaacatgatcaactcggatcttctccctgatgtcaaggtggcagggaagaacaggaagcccagagcttcttctcttaccctggtcttcacagcatcatcccgatgcagtccaccatcagccagttatcagtggacagaaaagcatggagtgcctaaatggttccaaagattacaaggctgtatttcaggaatcagaagtctcacaatcagggcagtgttgcatagtgtagtcacagagctcagttctcaagatctgcaatttataatttcatctgagcttcacgggggtgggggatgggggtgggtggggggtgatgtttcacagaacttcctggtgttactatgatccagaccctggagaaactgttttctgtgcatagttagatggttctagaggcatacagctgcattggtgtgtgaaagacagcctacagaacttgtctctgtcctaatttattctatcatgatatcaagcttatactcagccaagagtgggcacttcagacacagactttagtctgtgcagtacttagtcaataagcacttagattcactgtgattctatgttagcaaagtcatgtaggccaggctggagagatggtttactggttaagagcgttggctgctcttccagaggtcctgagttcaattcccagcaaccacatggtggctcacaaccatctgtaatgggatctgatgctctcttctgatgtgtctaagtgtactcatatacataaaataaataagtaaagtctaaaaaaaaagttttaagaaaaagaaaaaagtctaacaagccaaagagactcaaaagtactgaagtctgctttgaatgtttatgaagaatgtaccaagatttattactgcttgcattgtgcatgccactagttgcatagagaaagaaagggtgtgtttggcatgactaagaggaccctacccggcctgacatgggtctccttttcttactgatgaaaggcattttgctttattagcttggccttctaccttcttcacacagatggttgtgttggcatatgatcatctaaaaccctagcaccggggacaggaagacagcaggcctgcctgagctgcataacaagaccctggtttaaaaaaataaaataaaataaaataaaattgtgggggctggagagatggctcagtggctaagagcactgtctgctctttgaaaggtcctgagttcaattctcagcaaccacatggtggctcacaaccatttataaagggatctgatgccctcttctggtgtgtctggaaacagctacagtgtattcataaataaaacaaatctttaggaaaaaaaaatagagaatcctttaaaaaataataaataaataaaactgtggcggggattctcttgacaagcactcacacggccccaggttcttctagcaacttaaggagctaaagaaatacagtaacttctgggagcagaagggagatgatagttcatagactattaagtacttgcatagtcctttcttttatagtgctgggtatccaatcccgggctccacatcacatgctaggcaaatacttttacggctaaactgcaacctagcccttaggggaatcctgtcttaaaacttgtagaaccctgaataacttgctgtcaaattaatgtgctgacagaaagcccaggatcacagcactgaaagcagattttagaatcttccctaccttaccattgtgttcagaaaatcccttctgtagggtctttgctttatgatagtctagtttgttttcatgtatagtaacaagaacctgtggtccctttctgaagcctgtctgtggtgaagcagtgcagaacagaccactagcaccctttacaaaccctgctttgatgaatttttttctctgttcttggatagagatggctcagtttgacaagttctgttcatactatacacattcccagcgtgcacattcagagaacagactctcttgactggtgtcttctgacataaactttaactggcaggtggactctatacttctgtctgcattgaatcagaaaccatgtaaggaaaccttcatatggtagaggtagtcaagttttcagttcttccctcgctaggatgctttatctggaagctacagataaactctcaaaatgcacccctgacctcttcacctgcccagtcccagaagtgccttacctcaggcagggcctcagctagaactcagagggttttttaggcagcagaagaggcaagaacaaaaggttgcttgctcccactggttccattttttaagacatcagtgcctcatgaggtagaagttgcagccagtgaaagtgaaaaagaaaggatttttcccttcttcaaaactagttttggtctacttgagtttctgtcttcccactccatggccagtggattttctaattgcagacactccctctagggtacccctgacttcttcaccttccaatcctagggagagtttaatgcgtgcttctcttcagtctcttttcacttcctctttccacggtaactccccagggtctcagggacttggaagcaaggagtgtctttggttctgcccaaggcccagttgtcttgttcccagccagctgtctgtgtgtacagtgtttccgagcttctgtcctaggtcttctgtgatgatgtgttctaggccttgctgctctgtccatctgatacgatcttcttaaatgctcacattgtagtctctccaaccctcccttttaatttccccttttcttttctttcccttccttctctcttttgcttttttttcaagacagggtttctctgtatagccctggctgtcctggaactcactctgtagaccaggctggcctcgaactcagaaatccacttgcctctgcctcccgagtgctgggaataaaggtgtgtgccaccaagcccggctccctctctttctctccccttctcttttcattgaacataccagggcgcatgctctaccaccaagccacaccttcagcctagcctgaccttcttagctttgtcgttatcctgagcctgtattcccttcctgacgggcctgtctacatgagatgctttggtttcatgccttgcccactgtagtctctggaagcctgcagctttctcagagcccttctctgcctgacattcttctcagggcactgctgggtcactgactagcttgttcatttgacagttaattcattaagcaagtgccatactgttttgctccttgtggcgaggtgggatagtcaggaagctctgctgcctgggctcagggcctgccttcctggtctatgcctgcagagtatgagacctggcctgtatgactttaaaaagaaaaaggaaggcaaaaactcacctacagcttagtagaggaacacacaagggatttcttactttcttttaattaaattctgtatattacctctttaacttgtatgattataatattaaaatgaaggcaaaaattgcaagtataaattcatcagctgaattataagacttttttttactatttttttattacgtattttcctcaattacattttcaatgcgatcccaaaagtccctcataccctccccccgaattataagacttttaagattattcatgaaactgacttcatattggatcggcccgcctcacatttgctttctgcttttttcttcagtctgcatgaagaaatcagtgatttttatgagtacatgtctcccagacccgaggaagagaagatgcggatggaggtagtgagcaggatcgagagtgtgattaaagagctctggcccagtgctgatgtgagtacttgtcctggacctgggcttgaggcagagcctgccccagactgtctctcatagagactcactcacactgtaagtgctttggtagaggtagtctactttattttgcagcagggtcttacgatgtcatacccatggctagccctgaactcaagcctccttaagcttcccagacatgtgccacacccagctttggtcaaatcacatcttaaaggcaaacagggacctgcagcctgtagactttggggtttgttgatgactttcttccactcagatgctttactctaaacctcttcctctcagttctgagtagcacccaataaagtggacagaacttaggcttattaatgcaaggaaaagagaggtccagcctcagtacagtagttagttgctggaagggatttatcaaggcttcatctttgtttttactctagcccgtcagtgtaaacattgcagttggcttgtagctgacggcatttcttttgctacttgaagttgactgtcttgggcaaatgcaggcgagtgtatgcctctcattgcaaaccactctttgttccccgggccaacttcttttgattactcagtcagtgagcagatttgttgccttagtctccactttgtgtgtatctctttttttttttttttttttttttaagattatttatttatttattttatgtatgtgagtacacagtagccgtcttcagatacaccagaagagggcatcagatctcattacagatggttgtgagccaccatgtggttgctgggaattgaactcagcacctctggaagagcagtcagtgctcttaaccgctgagccatccctccagccccgtgtgtatctcttattattggcattaatacttaattttttagggattctttcaggtctgctgtagaatcctccctgagcaaagagtaactgactttgttgcatagattccttagaccttcctgaagtgatgtaagggaagccagggccaggtttgtccatttggcaaaagaaagaagttcaaatataggtaaagaatagaagggaaggcacaggtgcccttcactgcactggggcacagcagagatggctccgcctggcatgcctctgtaagcactgctgacatctagtggctagaaagggaagctgctggcagtgccatgacctgcacagaccctatagcaagggagaacctgccccaggttaagatgcctccatctttccgaaacctttctgaggtaggcaattgtattcatacaaatagaagtgcagagatacggctaggttcccggaactttttttttttttttttttttttttttaccagaatggtttagcattttcctgacttggcttacagacacatgtataattgcctgggtgcctctgtcctcagtggaccaagattctcagttgatagcacatcaggatcttgtatagactgctcctggtactctctcgcactgacctacacatcagcacatggtcagcagctaaatattctgaattcatgagagaactcctgcagagaggacagaattaaggatttgtgacattccttatcttgtaaaagaatactaactagcctttgaaacataacttgtgaaacttctgtataatgtgtacacatgtatattattatatgtatatgtaggcatgtgtgtagaagtcagaggacagtttttttttcccattttttattaggtatttagctcatttacatttccaatgctataccaaaagtcccccatagccacccacccccactcccctacccacccactcccctttttttggccctggcgttcccctgtactggggcatataaagtttgcgtgtctaatgggcctctctttccagtgatggccgcctaggccatcttttgatacatatgcagctagagtcaagagctccggggtactggttagttcataatgttgttccacctatagggtgaggacagtttttatagtcaggttttgtctacctctatatgtggtctgggcatcaggcttgcatagcgagcatcttcaactgctgagccatctcaatggcctcaacgtctctgatttagattataagccatgctctgagtgaatgcccgtgaggcatggtgtccctactttaggaaagccttgtatttggacctgtccattctaaaacaccagtagaggacaaaagggacgtgcatttctctgtaggcaccttaatgtgatttttcttcccttgttctaggtccagatatttggaagttttaaaactggcctgtatttacctaccaggttagtatgttgatgaagttttgaaggattattattttgaagggattattgtccactggggctattgaatatgtctgagtagaaatggggcttgtttgtttattgtttttgagatagggtctctgtgtagtctgggctgtcctaaagcttggtgagaacagttgtcataggtgagatatggaaaaggcacataaataatgagattggaagtaaatgcgtgtatttacacactgagagccagcatgatgctctggctttatgcagcttaatagtcaggtcacatctcattgaggcagcacacgtgaagtttaataccagaaggctctaggaaaaaccaaacaaaccaaaaaaccattcactaatagaatttaatatgtttgaggttttgtttctgtcagcattatctcatgtcctggggcctgagatctgtgaaatacctgagggtatgaggttgctaaaattgacactccctgcttctcctctcctctcctctcctctcccctcctctcccctcccctcccctcccctcccctcacctcccctcccctcccctcccctctcctctgcttcccttccaaaacttaccatcagcacactgatgctctgaagcagagagggaaagggattcttgttaccctaacttaaagttggtaagcataattgtcagctacaatagagacctgattgatgcttcttgaaacctcacacgtgtccctacagtgacatcgatcttgtggtgtttgggaagtgggagaacctgcctctctggaccctggaagaagcccttcggaaacacaaagtcgctgacgaggattccgtgaaagttctagacaaagcaacggtaagttcttagcattgtgtctttgtgagccttattacctgcaggagaaacttggactctaatctcgaatagcacagttggaatgtgactcagtgaatcattttttgaatgtcacagtgactatgcataaagactgtcattggagaatcaaagtaacttgtccaggctatacatttctttaagaataatacccaatttttcatgcgtaatgaaatataaaaatagattactagctgggcattggtggcgcaggcctgtaatcccagcacttgggaggcagaggcaggtgatcttagtttgaggccagcctggtctacagtgtgagtttgtttctgttaaatagcccctagttataacaaaagatattgttctcttattttgcacttgagatcttgtcaaggccttttttttttttttaattaaaaaggatatgtttttattttgtgtgtgtgttttggggctacagcagagggctttggatcctctagagctgagctgcaagcagattgagccacaatgtgggtgctaggaattgaacttaaactcactaagtgatgactcttagccattgagccatctctatagcctctgaagtcatgctttctatagagaagtcctgtcgcaaacaatgttttaaatagaatatattgtatttttatgaatttattgctttttagaatttttagaatttattgccttctatgaactttttcattttacttatgtgcgtgcacacacaccccccacgctccccctccccccatgccctgtgtaggtctgtgcgtgagaggacagtatggtgcagtttctccactctgagctggaggtccagctcaggttatcaagcttggcagcagacgcctttgcccgctgtgttggctggtgcctggtcctcagagcacttcatggttttgtggtctcgtgagaagtgtagaggtcacaggtctagctagtaaccttgtgtcctgattctgccacttactttgggagactattttaaacttttatgtcttctttttctaggttcctattattaaattaacagattcttttactgaagtgaaagttgatatcagctttaatgtccagaatggcgtgagagcagcagacctcattaaagattttactaaggtcagatacagtctatattgtgaagctattaagtttgtctcgagcgactgctgtgctgtgctgtcttctgcccagcttatgtatgcgtgcggctgacacatgactgcatttcgtgtgcttcctgtggacgggtttctgaaaggttgtggacacttgtgaagaccactctgttccttaggctgagtggcatgggagctgagattgtacaacaagctgactctggccagtttttaatctccaagtttagattagaatctttcccagggagcctttttttttaatcccaaattctctgtcaggagatacttgctactttatcttgaggtaatgttagtttatttcaattggtttcaaaataaagaagttgccagcattatatatactcagaacattttacagccccagacctgaaagcattctgtccaatgcagtcacctttcttaagcagttctccagtttctagacatttcagtttttgtattttggtgtttttagtttttcaaacaggatttcttatgtagccctggctgtcctggaactaactctgtagaccaggatggccttgaactcacagagatcctcctgcctctgcctcccacatgctgggattaaaggcatgctccaccactgccctgctttttgcttgttattttgaggcagagtctcactatgtggttggcctcaagcttttaatctttctagcacagtatctgggattgctgggatcatagttgtgtcctactgcggcttctgcctgctgtaccttgcttcctttttcctatcaggcatgaaagaatatagaagatagaaagaaaaagaaaaatatctcttttgcaggggctatgtatatgtgtatagcttttcataaaaagaaactaaacgttccaaattgtttgcttactaacaaacaatagaagatgacatgaaaagccatggccatcctctgtagcggaccttgtgttctgtgcctggagcgttaacagttctggagggttatatctgtcagctctaagagccctgctcatcttctgtaaagcaggaagtgatctgggtggtgacctgtccactgggtgacagcgggcaagactgtgtgtgctcgggcagctgcagaatggaataggggaccttgctgtacagcagaaggccacacacaaatgatttcttgactttcttactggattagtgccttaaatgttgaggtgctttggcctttgtgctatttctgttgtctgttttaagagacctttctgtgatcataataatatggttagatagttagtatgtctgttcagacagcatgtggccgccattatttacgtcaccatgaggagaaagccttttattacttagaagggtgactttgaaattgaatatattacatatatctaccaaattttgtattataaggaccaatttatgcccctggtgatgctttgggctattaaaatataggaattcattgaaaatgtaatttcatttctctttcagaaatatcctgtattgccatacttggttttagtattgaaacagttcttattacagagggaccttaatgaagtatttacaggtggaattggttcttatagtctctttttaatggcagtcagtttccttcaggtaagttgtgtgggtgtgctatatcagtgtgcactggaaaaaagataaactacttgcagagacatttggggagaaaatttaaatcaggaatttttacagtacttctcctttaaactgggtaccttttatatagtttagcaaattagtatttaaacattcctaggagagagattgagactttactaggtatgttaacatgtaatgtttggcctggtttctgtaaatgtgaatgcaataacagccaaaacacagggctgtctggagctcactggtcagtcatcaagctctgggttcagtaaaagaccttatttcagatggaaagtgattatggaagacagctgatgtcagcagcagacctttggctcctgcatgcctctctacttacacttagacattcatttgtacatacaagtacacataccacttgaacatacacacacacacagcagcagtgcgcgcgcacacacacacacacacacacacagcagcagcatttgtacatacaagtacacataccacttgaacatacacacacacacacacagcagcagcagcagcagcacacagagggactgggaaatggctcagtcagtaaagtacttgccattgcaagcatgagaatctgagtgtagatcgctaaagcccactaacaagctggacacagcagcaggtgcttatagtcccagcactggagagggagagacaagaggatccctggggctagctggcccaccagtctagcccactgggttcagtacttcagtgacagacactctttcacttgcctatcatgcataaagtctaggttccattccttagcaccgttatctgaaaaacaacaagtatggtggccagatgtggcaggtagatctccatgagttcaaggccagcctggtctacacagtgaatgccaggacagccagaactacatagtgagaccctgtgttgataaacctaaatatatatattataatatattgtgggcagtgacttaggaagacacctctcatcaactgctgtctttgatatacatgtacacacacacacaccagaaataagagtacatttaaagcagttacccatggaagctcagctataactgacacatgttctttggataagagctttagttttgcctgtttggcagtggttgccagtaaccagactgctaagtgagatgaattccctgatgtggctcacactcagccttacctacctggtctatacccatggtgcattgcatgagctttgtagcatagctgtcaactggtccaagatgatgatgatgatgatgatgatgatgatagcgagtgctcgtagaagttgggatctcacttataccccctccttcataacagagaaaatccaaaataatttcttcctttttttttttttttctgttttagttacatcccagggaagatgcttgcatccccaatacaaactatggtgttctcttaatagagttttttgaattatatggacggcacttcaattacttaaagactggcatccggataaaggatggtggttcctatgtggccaaagatgaagtacagaaaaatatgcttgatggctaccggccgtcaatgctttatatcgaagatcctttacaaccaggtactgcgattcggtgtctgtaggcttcagaggggcgtcacagtccacccatgctttactctgttcagatgaaaatgcttatttctaagtagcacttgttcagaatattcttatcatactaaaagagacacatactccagaaaaaccacctgctagacttgcacataagttgaggtagtgtccatttgactttcaaagccaagcagagaggaattcactgtggggtgggtttgcagccatctctcctcctgtgggctgccatttccagctagttgtcagcagggctttagtgaggtaagatgggggctggagagatggctcagccgttaagagcactgaatgctcttccaggggtcctgagttcaatacccagcaaccacgtggtggctcacaaccatctgtaatgggatctgatgccctcttctggtgtgtctgaagacagtggcagtgggcagtgtactcacatacataaaataagtaaatctttaaaaaaaaagaaagaaagaaagaaagaaagaaagaaagaaagaaagaaagaaagaaagaaagaaagaaaagaaaagaaaagaaaagaaagaaaggaggaaaggtggaagtcctcagacagcagggagcatgggcagaaaagctccaggagaccgctcttactcatgttctgactgctgctttctttttgtgtgccctctctgtatggctctagctttgggagtggcttgagagtgaacggagtgcaagtttctgcctcactttcacaacgttaaatgtatttgcttatgatgtgcagatgtatgttcatagctaattcagacaaagaccaggcagcacccttccttccttactgttttcaatgtagcttggctttattccgaggagaaaatagccagctgtttgtttgtatgtgataggagaatccttgctgggctaaagttgtatgcaggcagtgggtgggtgtaccatagcaaagcactgtgcaggtcttccaaggctgacagcccactcatgagcagctgtcacccttgttcctgggcaagggctcatcagcctttattcataataaccagcagccaggtttacttgtcttcgtatccatttcttttaaaggtaatgatgttggaaggagttcctatggggccatgcaggtgaaacaggcctttgattatgcctatgtggtattgagtcatgcagtgtcaccgattgcaaagtactatcccaacaatgaaacagagaggtaaaagtctagcccaggccagcctgtgtgttgagagtggttggtacttcttatcttcaacttaatgtacacctcttttgtttttttttaacctgtgcagcatattaggtagaataattagagtgacggatgaagttgccacgtatagagattggatatcaaaacagtggggcttgcagaataggcccgagccatcatgcaacggtaagacctccttgatggtggactgggtcttagaggctttttctatgttttgtgtatttaatgggaagaaacgttttccaatcttttgccactttttcaggaaatggtgttaccttgatagtagatactcagcagttagataagtgtaataataatctgtctgaagaaaaggaagcccttggaaaatgtagaagtaacgcctcggaacctcttagtaaacactcttcaaactcttcatcaggtccagtgtcctcctcctctgccacgcagtccagctctagtgacgtcgtaagtatgacacatgctgccccagcctgctctttggagggccctcacaggcaccagggatatttccaatacctttcattcttgtactttttccctaacatttttttttaaagaaatttgaaaatctagtgaaggtagaacccgtcagtggctgtagcattccatactccattagcattgacctgcttattatattcctgacactctgtcccgtaggcagctcctcttcctctttgatctgcttcaaagatggcagatgtcacacttcaccccagcctccatagcatgcatcctggatttagatggcttgcttattctttattcctcactaagttttatgaccatgctcaggtcactcagagctctagtaagagaatgtcctctcactaacccacagtccccagtctcgcctccagagggcatccttgtccttgtaaaggattttctacatcacatattggttttccatgtcggggttcctagagctttgatctcatgctatagaatattatttgtaaagcagctttcactttcttttttttttttctttttttcttttttgtttgtttgtttttttgttgttgttattgttgttgttttcgagacagggtttctctttataaccctggctgtcctggaactcactttgtagaccaggctggcctcgaactcagaaatccgcctgcccctgcctcccaagtgctgggattaaaggcgtgcaccaccaccgcctggctgcttttactttcttaagcttttttttttctttacttgtttttgagatccctaattgaaattgttaattgataaaacttgcatttaataaaagtataattcagggttcccagcaatgatatcaggcagctctcaactgcctgtatgtaactctaggagtgtgtgtgtgtgtgtgtgtgtgtgtgtgtgtgtgtgtgtgtttgtgtttgtgttttctggaactcactatgtagactaagctggcctcaaactcagattcaactgcttctgtctcccaagttctaggattaaaggctcagttttgagtttctaatgtattcagtattacacagctactttcactcttaatttaaggatgtttccagctaggcatggtggcacatgccttcagtcctggcactcgggaggcagagggagacagtcctccaccagggccatatagagagatggtgtctcaatccccctgccttccccccaatatccatgttatttagtaacctctcccagctcccctttcccataacctttggtctacttcctgtctctatcatgatgaataacatctcacattaaattttcgttgtaaaaaatttgagtctacttaattaaagtttaaggtagatttgtctttcctttgaattttgactttaatatgtgattgcaaaatgtcctattagggtcatggaggtactagcatgtgccctgggtttagtcctctgcactgcataaatcaggcttggtagtacatatctataatcccaataatagggagggtggaagcaggagactcaggagcaggagttaaagacatgctctgttgcagagtgagttggagcccagcctgcattaaatgagactgtgtctcaaagaaaaaggaagaaagaaagaaccaattttccacatagcaatatgatggcatattgttatggagtcataaatactgatatactggatccttgtgtctgggttctggagtggtgctttggaaggtggaatgaatgctaagctagatgtccctgtagagactgccttgaaaagaagaatttagctatgccgctgacgtgtcaggcagtgtaccaacataaagcagccaagagagcagatggagcttagaacaagcatccctgtcctagacactcagttgtgccacttgaacctgccatcttgcaataaggtccctccctctttatggtgatcttccattgtttatatgacttgctattttaaaaagttcacatcaactggaactcacaatgattgacattcacaactgatttaaaaatcagctagatgtggtcagtgacttggcaataactagaagcatacagtagaatagatcacctcacatgtctgcagaaggggctgggaaatgactcaatggggtagaggtgcaagctgtgcaagcaaaaggacctgagtgtgaacaccccatgtcctctgacttgagtgtgaataccctgtgtcccctgacctgagtgtgacccgaatccctttgaaacagtcatgactgtgtttgcctgcagctgtactgttgggagcagaagctctcattcagtgagggaccctgtctcagaaaataaagtggaggggtagaggaagacagaagctgttgtctggtctctgactgcatgctttggatgcgcacacacaccttccttcacacagcatttgaagtaaagcctcctcaggtcccagatgtgtctgtttagactagaaagtacttagccgaaattgttgcccctctgagttctgaatatgaaaattactaatatattgctttacccatgtgacttgtgttaggattctgatgcaacgccatgcaaaacccccaaacagctgctctgccgtccacccactgtcacccgggtaggctcacaggatgtctccttggaggtctctcaggcagttgggaaaatgcagagcactcaaaccactaacacacccaacaacgccaacaaatcacaggtgggtagaatctgtgcttgttactcttagcgttcagttttagatagttagaagttcttttttcgcttctaattttaaaatgtgtatttattaaagcatttataacaagtatgaatagcctatccttatttactagagttgataaaaatgtccacggcatttgttgcttctttccatgtttataagctccatcaaacccacacattctcacgcctctactaaacactaatatttcacaggggtaaattttttcagtactttctaaagtattgagaaaatacattattttgttgtacttactgttgttggacagatcagataacagttgtgtttttatagattgaaaatcacataaaattatccaattagaattaaatttcaattcttgtctcatactattattggaagaataaataatgaaacacagcctgagaaagcagaagtcataaagtggcagcatggatagtgtcccgttgactagcacgggctcaggtgtgttccttcctgttctcagcagtgggaagtgtggccttgaaaagcccatctggcccctctgtttttctcatctgctagacagacttaattatgtacctcccttttctataaaatgatgacaagaatagtctaccttcaaatttatagtgaatactatgttaataattatagaaatgcttagaacaattagaaaatgttgagcatatattaagcatcatatagttagaatttcttacatgtgcacagttgctacacataaagaactctcttagttaaagaatgaactgttttgtcaaaccagatgtcctgaagctccttttaagatagggttgtaacatagtaacatagatcttagtgctagccaagcccattgagcattggggctgaggatgtagctgagtgtagagcaggctgatgagtggtctccgtggctgtgcttgaggtgcactggcatggaatgtctgcagcatccctagaaacagctagttctttgttcttatttgatacttgttgctgaggacaattaccaggtacctgggagacaggttcagttgattaacatcagctttgacttacttaagtatttttagttgccaaatagtattactaatttgccatgaataactaatcaggcaagagaagaagcttttgatactgatctgtggtaaaaattttctttcccatcctcagatgtagcaggttccataggaaaaggtctgtcttcttggaagttgtgatttgattctcccggttcactgtacctccacagtcttagcatgggcaccaaatgcaggtgcagggaggggcaggactaaccagagactggtgccagtaggcaccacaagagctgtcactgggaacaggaaagaaacacctaaactgtttaggtctcttgacctcctccggtagtgcacacaacatgataaggaggaggtaaggtagacctcctccttatcatgttgtgtgagcagtaggatccttgaggaagctttcatgttgtacacatgctgtcaggaaggggagctatagagttcacccctgttagtcctcgttttctagcctctagaacagtgtgtgatagctgggtgtgatggcacacccctttaatcccagcacttgggaggggaggtaggcggatctctgagtgtttgaggccagcttgaactgcataggaaggaagggccagactggccagagtgacagtgtgggagttgtcagtgggggaccatgttaagtgcgcgcctgcccctttctagtctgggagcggccctgcatgcggtttgccagggggcagctctgctccgattctcactgctttcttcttctgcagcatggatcagcaaggctcttccgttcttccagcaaaggcttccaaggtacagctcaaaccagccatggggccttgatgacaagcaaacagcatcaaggcaaatccaatactcagtattaccatggcaaaaagaggagacacaagagggacgcgcccctctcagagctttgtagatagtcggcgctctgcgacagactgtcttctgtgtgcaatgatctcgtgctcaggacagttgcacagggactcctgggacggcaggagcctcacactgttcagacgttgatttagcaactgcgttttttcccagctcgccacggaatggatcatgaagactgacaactgcaaaaacaaaaagcaagcaaaaaaaagggggaaaggctgcttatgtgataagtcatgtgctacaacagggtcattttaagatttaaagcttgaatgtaaaataaatatatttctcattggctttatgcagagttataggggctagtgctcagtgtgggtagctgacaggaagagagcagtgtcaaggagatgggtgggcaggtcagcaggagcatctcatgggaagtcagactccgagggaaaggagtttgtgcatggttttttttaaaaaataattttgcatatatttgccattttattgtgtgtatatatagaagaccatataggaaattgatatttgtaatagtggatttgttaatactttttacataacattactatttgaattgtaaacagatttttttctcaggattagtttgaagaataattgagttgtcactcttaacacatgcagggaagtgattagctctggtcctgtctgttttcttcagcattgaaatgacttcatagaacccttgtgacctgcttcaaaattctttcctctctaagcaaaaggtttatggtggcaaatgatgtttattttattttgtaaaaaagagaaaaatgtactgtgtacttgtgtatacactgaacaacctctagctgtctctccgaatgaacacacctgctctggatccagtgctgttgtcttcctgggcactgggccgtagcaggccttgtgtgtgtttcctagcagtcttttcttcccctccctcctcttcttcctcaaaggaaacgaaaggcctccctgggcctgggctgttcctgtcacagtgtggctgacctctagctccacagcccttgttagctcgcatgctggttcctctgaccctgcgttccatgtgcttgtggcgtcctctcattctttctaggttcctgcttactgctgtgggagagagtaactgtaaacagctttaatgaaatcatacttataaaaactattttcttatactccactttatgcttttggtattgtcgatctttaaaaattaaatggtctttgataatggatcgatttttgtattgccttattaagaccaaatacttcttgtcatcccattctttatcctctcctttaatggaattgctgtctttaattaaaactttgtaaacactggcttgttttaatcatcctgtaacttaggctgtggtcagctacaagcgcagatgtgtaatcctgttactcattgccagctgagtcttgagactcgggtggggtacttaacacaatgcatcgtacacgctctgctcgctcgctcgtcagcagagtggtcttggaggtgaaagccctcgtgtctagcatcagtgtgtgtgtgtgactctgttcagtacggccgtttccgagatgggattcttttatatgtgtatgtgtgaagtactgttggcatttaggcatttcttttctatacacttaggaaatactgaagaccaatcagaccattaatggacacttagtgcaactttttataatgagaataatgctataaagtaagaccaaaaccggtgtcatcactgaaattaacaattttcaatatgttcatattttaatttacaatgggaaaaaatgtgttccacaactggaaactcacagtactgtgtaaactgtgtaagattttaaatgtgatgttattttgactgttctcaattttagagtcacattttattctgatcagaatttttatcaagatgttgaagttttgttgttttgaaactagtttgtcataacatattgtgcataatcacagtatttattttgtagggcttgtggatgtgtagacttatgtttactgctaagggaacaattatttataaaaaaaatattaaatccagtattagctgcctatttcagacacttaacacctgcagagatctgtgttacatttaccacactgaagtttttttaaagaatcaccctcattgttgaaagtaaatgtactcttaggtgggttattagtgtccaataagcatgtgattatattaaggtggtggtagcgggaagataatcttgattccattgggaatcttaggttttcgtaaatttattgggaaaatagtttttcctgtactgctgatgtttctttttggtaaacagtatctttctaaaagaaaaagcatgaaggagaattgaggtgtgtatacatttccccagatgaccagcattgtattcgtgaatactgtgtatctggaatgagcagtgtgcaagctgttcatttttcaatctgaagtaaaatactttcaagaac

SEQ ID NO: 6 - DNA, 36659 bp, Mus musculus
  source 1..36659: mol_type = unassigned DNA
ccgccgcccccgcgccgcctccccgcccctcattggagctgaggcggcggcccccatccctctccgttcccggccgccgccgagggcgtctttccgccgccgcgccgcaggagcgccgtgactgactgacggcccgacgctctgggcccgccccctcggccggtcaccgccccttcgatggcctctccctgccgcccgcacgctacgccgccgccgccgcggccccctcgccctccccgcgccgcctgagccaccgggaccgcagctgcgccgcgcgccccaccgagcgccgcccgcgcccatccgcgcgcaccggagcgcggcccaggcccgtccgtccgtccgtccgcgcggccggcccggggcgcggcggggcggggcgcggcgggggcggggcgcggcgggcgacgcgggccccgcgggggcggcgcgtggatggatccgcgcgtggcctggatccagccggagcagaaggggccggccaatgccctgtggatgcagatctgggagacctcgcagggcgtgggccgtggcggctccggcttcgcgtcctacttctgcctcaactcgccggcgctggacacggcggccgcgcccggggcggcggggcgcgcagcaccagcagcaggaggcccggggccggcgcccgccgcctcgtccccgccgccggcgcccggccccgccgcgctgcccccggcgctgcttaccgcgctagggcccgcggcggacagcgcaagacgcttgcacaagtccccgtcgctgtcgtcgtcgtcgtcctcgtcgtcgtccaacgccgagtcgggcaccgagagtcccggctgctcgtcgtcgtcctccagcagcacctcgctcggccgcgccggcagcggccgcaccttcttcagcttcgccgacggtgctgcccacgcacacccgggcccacgcggctccacgcccgccggctcgccgccgcagcaccagttccacccgggtcggcggaaacgcgagaacaaggccagcacgtatggcctcaactacctgctgtcgggcagccgcgcggccacgctgagcggagggggcggccccggggcccaggcggcgcggcccggcacgccgtggaagagccgcgcgtacagcccgggcatccaggggtgagtgcgcccgagccgcggggctgccagcggcgcgggcactttaaaaactccgttggatcgagatccaccaggcaggggggattgacagttcgggatggaggcctagggctagactagaaagtggttcctttctttcctctgctccagggcagggaagcaagggctggctgtcagagtgggaagcgtttacgcgtgttctttcctgtaggctaaatcctgtatcttagtgtagtgaggttgaatgtaaaaaatttttgttccctttttttgagacagtaggtttttggtattggtgcagtttagccatcaagtgaggtttagaagtggagtcttgtgtgtgttgaggttacctcgtacgttttaattttgggcagacgttcacagaaagaccgtcacctggggtttctgaaaaccacctaatagtactcaggtttttttcctgtgaactcacaggaaggtgttttcatttaaaaatttttccataaggctcagaggaccttagcggtttaatgtccccaatgacagcaaggtgggggtgttggtctggaattgccccaggtgagacttttctagtatgaagctcttcattcaacctattggttgaatttctgaacatctagaaacaaaatagtggttgtgtagtctcaagactggtgagcttgatttgcctcagttcctgccgctcttaaagggcgtctaacctccccccctccccctcactccagggagcgacatccccagttcaagtccacccgggtggttgctaggccctgggactcctttgaggtaacttcttagggggctgtttgcggtttgaggtctgtggggaacacggatactctttaaggggttgggggtgttacatagaaacagttgtcccctctgcaacttgtttcacctgccttggatctttgggtcgggattctccagcatgcactacaaatccaggaggagcctggtttcttctgccagttcccccgcagtgcttgcttgtagaactttgtgagacaggtgtggccgtggacctacgggaggtttctggattgttacataactgtaatggacatggtgtctgccgggagacggatcctagcctctgacagagttccacttgggtacttaggcatcagcagcatgatctgcccctcggtgagagttgtgtcccttgatgtggtctctctggtggtctgtaaagctggtcagcctctgtgtgttcttctgtggcgttttgttagtatgtgtgcctctgtgggctaggtgtgcttttagagtctagcatgggtgcagttggcatgggtggcatgtatttctattgatcccacaagcatggtcccttctttaaataccctcccttcctggtacagatgaacattgccacctgggtattcatacttggctcctgtcaccctactcccaagtctgtagttcttgattagccgctgtcccccaagtctgggtctctcttcctatgtcaagcacattgtaaccactctctggaggaggatgctcaggcagtccaagactggggtcttctgtgcactgtggcctagcctgtgtccttggtcagcagggtcagtgggatgaggttctaccagcccccaactttcacgagagcctccattacttcccccacccacccaagagagatgaaggcctgactcggtggtgcttgggctctgccactcttcttgcctgagccctgcctcagagccccacttgctcactttagggttcaaaacccagctagagtttagcgttggatgctgcctggtgactcttccttttctgtctaggtggcatgtgttgaatggtgactcctggaggaggagcttgctaactggcttggcatgtgtgcgtgagagagtgcctcctgagagggtaccttgtctgtttcctactgtgactgacctctcgagggggtgggaaggaattagtgaacgggcaccagtagagctgagccctatatgttaacagtaaaggtggctccaactattgctggttgctctcacgtgagctgatcttggatggcaaaaaaaggacctgtctgaagctagtcttgtgctgtgtggggccagcaccaagcctggcttagcaagagacacaagtaaggttaagtgttggcttgagcgtggcccagttaacagtgagggggcctgttatgttagtgagaggctgctgagcttgctttctgctgctggggacgtgcctttgggggactccgcagacctccagaaagggtgagtggttttctggtggggtgagagtctgggtctctgcccaggtggacacggggcacttgcacctttcagctctctgggtggagtttgcccttggaaactgctagactgctttcgatcccgggtaggatgaagaaagcggacagggcttagtgtaatgagcttggggaggagagaaggtgagccctgctatgggagctcccctgagcccagctctgaaaccttccttgggacatggcaggcataatgtcagatgtgcctctgcatgagtttcttgcttaggatggaggcaggcatgggcaggctctctgggtcctccctcccccctttaactaactcactgtgccttctgagtccttaactctgacccacagtgtgattccttagtcctgggcagtctcagtggctttagaagctgctttgggttaaaagtaaagagcattcccctggaagtgaggattggagtgtggtcctgacctccctggtgtcttgcaggagtgcagatggacaggggaggccccagcccttgcatacacctgcagccccatgacttgagttgactctgaaggttgtgtcctcccaggtgtgcctacagtgggcagcttctctgcaactctcttcaccatagttagtctttgaatgtgtcctgtgcccttggcgacaggagtcccgttgtccgtctgaagccggagaagcctccagtgtcagcagttacagcttggctgtgactgatggtgctctcagggctgtggggtgattccctcccagctccatgactgctctttttgctgttctcctgtgagggagtcagagctcggtggagcagccaggttccctctcctgcagcacagctcctggctgttctgacagaagcagatggcctttcctcagaaggctccattgggaaggcacaggtgagggcccagtgggttgtgctgtggaacacaagtgtagtcactgctatctccctccctgtcttccagtgagcatcaggggatgcacgggagggaagagggaatcagggaagctgtaaaccccgcctgtgcaggtgacagtggcataagaggtttctttcacagagatgctcaaggttgaagagggagatgagaacagtatactacacagagcttgttgtcatatggacagtgttacctcctgtgcaaccttctccaactaaacagcaccactctcttgtcatggccagtctccatacagaactggaaaggctagtgagatgctgtcccgggcagctgaggggagtcccggagaactgctgcaccacggcttgctcaggatggagagctgatgctggggaaggctgcccaggaggacaaggccagtcttctcttagactagaatattaagcaggctggcgcagtcctttggaaaagaggccctagggttggggaggccttgggtttggttcctaacaccaaggagaaaagcaatagaaacaaagaaacaaacaaccataaaaataaaaatggagtccctgagtaattttgaagaaaaaccatctaatgctcatgctgtgtaagttaaggagggcgggcatccgggtgtcctggcgtctgggtgtcctggcgtcctgtggctgggatatttggatgaagaagcagggctgcttcccccttagaagcaacacacctgtggcttaaaagtaatttttaagtaaaaaaatttttttagattttttttcatactgttatttacatatttgattcttgtgtatgtgcttatgtgagtttatgtgcatcacatttacaaatgtctgcggtcgccagaagagggcattggattcctctggacgtgtaatgacaggcagctgtgggcagccagatgtgggtgctgggaacaatctggattctctgcacgtgctcccaactgctggacagcccatctagctctttaagtagacatttaaaaggaaaaaaagaaaatatgactttaagacttgtgcttcaaagtcttggttatttttgatgacagatgtgagcttttgggcatcaccttgaaagatttcataccaagtatcctttgggagcagggataccagtcagtgcaccatggacagaaaggatgctgtgccttggtaaggtggtatacctaagaaagtaaacccggatgctcacattcagagacatggttttgatgatgcagacagaactcaagctgcaggggtgcccgccctttccaggaaccatctctagtgcttgctatggctggtattgctgtgatgaggctaggccagaggctctgcccagcctgtcagggttgctttaaccttaaataaagttggatggtatgctaaccccataaggctgcccaggagagtgtggcttacagcaaagtgaagttggtgatgccctctgcatgcctctgagtggggcagccatgtcgtcttcccatttggcatgtgcacggactcagacagcattgaactcttgatacgtctccattgttcactgtttctgtggtcaggtagcaaagtccagtgcactaggcttggaccggttatctcatcagtcacatccttccggtcacttgaggatggcttgggtgagttctctattcagcagggcagagggctgagtgtcagctgcttgttgtcacatctgtctccagagccggctcataagcacattcctgtagtttcagcacttggggagactcctcagagggctcacatgattgcataggccaattcggatggtctgtcttcaggccatgcaatgcagtgtggtctatagactattccatcagaaccacaagtttgggcatgggttgctgtctgccacacactggcattgtttagtaggaagacagcattgatgtgggaagaggaggccatggttgtcttgtccaacactcttgtcctctgcttggacatcagcttatcacaagtgactcttttatattgccagtcccgtcttctggcttgattgtcacccctgaagctgggctaattgatgacaagaacagtgtgcacttttctctgcctctcacagatgaaccattctttagtgtgccatttaacaaagtctatacaagagccacgtgattgcacaaaaactgtttcagccatgagggcacctgcattcatcagattgtcaccgtggcccatttaagttgccacttgataatgacaaaacagcccatggttgctctgtgtaagatgtggcccacaccggatctgcatgctccgttgatggggatggtggtttgcccatggcctcagctgcagaacatgtgcctctccctggaggagcaaaaagaccccccacaattcttgccttgtctcttcctgagctcccatctgaatctttagtttctacggagttagcttttactgtcccctgcctacctacactcccagaaccaaagacgtgcatactaagaaagatgcaaaagacaaataggttttgtatgcaaaagtaaactttagttttttgtcttaggcattttaacatagattgtgagctttgcagtgattttttgttgttgttgttgttgttgttttgtacatgcaaaacagtatgttagtttctctctgagtgtagctaagacccctagtgcaggatgaaaacattaacatacacacaggatgaaaaacatgtagagaagtctcatgccacatctgaaaggtattcctatttgctggcatccttttctttttaaatactggataacaaccaaaaagtcagagaacctctgattggaccacctgccgagagtctctgtcatctgtgttgcctgttggcctcccgcatgagctggctgggctgtggcaagacttctgcacaggcttatcttttcccagatgacaggcccaactagatgggatgtcccggacatccttgagtcagtggcttgtgacactcatttgccttttagaaaattttttattttatttatttacattccaaatgttatccccttcctgttagctccagatttctttgccccatcccctcttccctttgcctctgagcaggtgctctcccccaaacctcacctcttaccccccccccccagcatcccccttccctgaggcatcaagtctttacagaatttaggcacatcctctcccactgaggccagacagggcagtcctctgctacatatgtgctgggggccacagagaagcccatgtttgctctttggttggtagcttagtctctgggagctatgtggagtctgggttagttgatactgttggtcttcctatggggttgctgctttgcacaactctagcttttcctggtgtttgttttctgagatacagcagtgggcatgattgggacacagagtcaaattagtccctttttttctttttttttaaacctcattttttttctcatattgggaagcatgcacttggtttggagttgactttgtgcaactttatgttttacttgaaatagtttaacttggtaagttactgaaataaattgaaactcaacttcattagcccagccacatacttgggggtctggttctggctggttttacctgtgctctaactcccacacctcagtttccccacctgagaagtagggcctgtgacataactcagtcagtttctgagctggtcagcatgtgtgagaaaggctactccttctccctttcctcctctcaggaaatggaaaccaaaaatgagcttcttgttgccaacaggaaagccttcctcttcttccttttcttaaatgacgaagctacagactcaaagctccacatcttagagaggggagagggactcagactcagctgggagttctggaatccccctgccccctagtccttgtattttaaatgcagacgacactttagcccattgtcacagcgtgtgtgtgtgtgtgtgtgtgtgtgtgtgtgtgtgtgtgtgtgtacaccgtgtgcctgctgtgctgcagctggagttaggttgggttcagttcttagcctctgctccgaggggaggtttgagtggccgtggatgacgatgacccttactctgagttctttgccacttcctacacagagttagtgggtttagagggaagagagcagtgtgctggggagccacgacagtgcagggtggcacagtcctgtgcagaggaggagatgcagtgtctgtcctgagtagtggccacacttcatgtgtcctagctagcacttggagggtggccagtgcaggtgaaaagctaagttgaactttaatgtgttgacttgttttgtggcattgggaatcaagctgagctctttgagcatgccaggcggaccgtctaccagtgaggaccctcccgagctcttacttaattttagttaaaatttaaatggccctgtgtttgaacagtgctgactttgtgcaactttatgttttacttgaaatagtttaacttggtaagttactgaaataaattgaaactcaactgcagtaaaaagagtgttgcttagtaagttgctatggagaaagctctagagttcatgtttctgagtttccaatgcaagtcctttatgaaaggctgatatctttgtgccggaggcagcagggaacggggcagtgcagcccagagcattctgaggaggggagcatttctggtcagcatgcaggagcgagcgagtaggcaggcaggctgctggtggtgtggcgaatgggttgccttcaggtctctgcaggtgtcacagcttcccgccacatctcctagcagctctgtgctcaggattcctggaggctagtgagccctgctccactgtactctttgacgtcctggttcaaagccacacagaggcaatatttcttgtgttttacaggcacaaacccagtctagattagctaaaatagtctgaccaagaggtctgcacttgtgcatattgctgttgctgagggaaatagctaccacagactgaggatttagcacagcactaggcagagccaaaggtaagttcatgcggagcctttcctaatgagaacaggcaaatacagagcctgcctgtgctgtgcttcctctgtgcagccgcgtcctgcagaggggcgcagggagccctttggggtggatctggcttcctgggctgtttagaggttgaattttgggagaggtgaagaagctgtgagttctgacatgtcctggaagggccaggcttccctgcagtcccaaaacagctcgtttgaacgagatgagagcaattttcaggagatgttctgattttgtccaagagtcacctaggaagtgatagaactaggctcagatgcaggcttcttgcctggcaagtccatgcacttgaccagtgctttccagagagtgagagggaacacgagttgagagtcagaagcatgacttggtgaaatggcagagacaagttgaagcataagaaagagtactttggtcacaggcatcttctctcaattgaagtaacattttagaaagtgaagagcagctatgatatggtatccaaaagtaaagagagcacttggcatgataaagaaacaaaggaattcggaatataactactagttatcttgtgaatgagtcatgagacactagtgtgggcatggaaggaaggtcactgtgttggcatgacactctgaactacagggtgggctactgaacagtttgaagcttgtggggggctgcttggggctggaagaaagctcactgtgcatgagactttccttagcagtgagctctgcctctttcccctttttcccacctggctgtagccggctgtactagctagctagagccttactggctccactgacccacacctgggccaggcctctcttttttatccctttatacagtgtttccggctgccctccaggatgctccaggaggtatggcactgagagaaacagtccacccacacaccacaacacagcacagggctgtcagtggagctcaggcctcgtgattccctagctcactcattacctaggaactaggtagagggacttgccaaagaggtgaggttggtaggagcaatgcttgtgaattccaggctctgactcctctggagagaggccagatgctgccagaattctttgcttctgaggacaggtctttgaacatcccagaggaatgtgagtgacacttctcactaatctagcagcagcaggacagggagctctgtcctacttgagaggggctcttctcctttctgtaagacagtttataggtgactcttcagagcattgatgcctatcgtttattcactgtttcttacataagggtaccccctgctgatgtaaacttaaggctgttctgtctcagcctcccagcttccaagattacaaacgtgagccatcatttctggcaccaattctaaaatactgatgaatcggtaaaactggctcatatttttaaaagtggaagctttaaagtatctcagtcctatcacccagaaatcgccaggataattagatgaccacagctgcagatggtattctccaagatcctggagatagtacttggagccaccaacagaaaaactcactagaatcatcttcatgaaaactctagtaaattatttattttgcttcagtaagaaacgtgcagtttcaaatagaaacccgggctcctgagagcgagtgctgtggctgagaatgtaaactgacaaagcccgagtgcagaaggtcgtgctaacaaggcaccaaccggtttgctctcagtttatgggctgagaagtccgcccttctgcatcttccctcctgactgaacatgaggcgttgctgtgagtttgctattttgttgtgtggtttcattcactagtatttgttgattgagcccttactgagccagtgtcgcaggcggtgttttcaggaggttcattgctggccgtgctgctcctctgtgctgtcctgtgctctcagtagctctgcagggccagccctgcatccctgggctctgggttgctctgctctgaccctgtgtgtgtagtttttgcctgagatgtggtttggttcttcatactgcatagtggctgctgccagtgtctttttatgaagggactggatagatgcattttttgtagagtggataagggctctactccctgaagcctgttgtggccctgtagggttgcttggctttcttctcccatggcagaacagtgccaccaccacctgtcctccgagacaggaccttgtctcctctggtctgaagattgatactaattgctgacctgtctagacatctctgtctaagctggcctctttgtgaggcagacttttctggacctcagatgcttgtgcactgcaggctgaagttggcctgcagtctagggggaggctctgtcaggtccttgctcctgtgttgctggctttgccttcctggggtgacaagtgtctgaatgtctgtccatctttggatgctgcttgccttggctctgtcactgacacccagcagcttcttcctgactcctgacttttctcaacattagggacacgctctgattttttttctgtggaactggcagtgaggtagctttctcttctgggttcactgtttttgtgatacactgtgtcccttatgccatccctcttactctacctgtgcacagcatatttcccccctgctcattttaatactcaataagtcttaatactgacttgctgtggatattttcctatggaagcagccgccacccccaatccatctgcagatctgaaagtctgcctgttccatgggatgactattctctggccttctggtaccagattgtgggcagcagagattcaggctgagctagggcattaccttgttgggacctggactctgctttgagttgtttcagctttccagggtcagtgctcttggcttacccctgggcttcctagaaggccttctggcctcttgtttacagtcagcccctggggacaggagtggccctgggctccagtgctcagtgctgctgtgtagggatgctgccttctctccagggtttccttgcctgggggcctgctgtttcaatccagctgctgctgctgcttcctttctcagccttagcaccagctccttggaaggcagtccctgacaactgtcctgtgccacatgaaaccagtttggatttcttcactgtttattcatttcccctagccccagggaggacccttctagtgtgagcaagggcctcagtttctcctgtctggctcactggtctgcagaacgagccacacaggagtaaactactggagtatgcaggtcacttctgctttctctactggtgtactgagagaaacctttgcacctgttgggttattttctctccctctgtcttaattcctgccttttctttctatatttttggtaaatttccactgttagggtctcccttttctcataaggtcctttttgtcatcttgagtgagggcagaaactactgccctggtcccagtttccgtatgtggggctgtgtgcagagtggttgacagtggtaccaggactctactcagtggccagtgtcacacaggagaggcccaagcacctgtcacatgcaggtgcagccgtgatgcttccttccagtctgaatgcagcagcagagctctctgcatagtgcctgggcttgtgtctctaaggaggctgagtggctggtctgctgagagggagggagtggctgttcagggactaagtcattgagaatcacacaccctccatgtggaagggtttcctaagcaaggaggtatttctgagcaagatgggtgtctgtgtctgagtttgttgctcatggcatgaaggggatctgggatgtgtcccttatatccagtgcaaagtggctcagtcctgctctctgctgcatttcagtatgctcagtgcctaagtactggcactgcttcatctggtccagcgcttaccagacttccctctatagagaccaaggggctgtagtaggtagccatgtcagaggcattgctagcactgcggcaggaagtgcgtactctagttaacgctttgttcatggcctacctgctgttgagaagtgatgctcttgctttgcctgaaagtggcttctcggtgcccttggggcttgctctgcctggggcatagtcttgagtcttggagcatgcacatagagcctcttagaacccctccctcgttaggctctggaaggctcttagacatctatctgttcagagtccagattggtaatgttgatcagaaagttgaggagccctcccttttttttttttttttttggttttttcgagacagggtttctctgtatggccctggctgtcctggaactcactctgtagaccaggctggcctcgaattcagaaatcgcctgcctctgcctcccaagtgctgggattaaaggcatgcgccaccacgctcggctcgagccctccctctcttatcttgtgtattaacctgttgtaattagcttttactgtaaacttgaccagactggtcagtgggcttgtctgtggagacttggcttcttgttaattatgtgagagggaccccactctgtacaagacaaacactgttatctgggcaggtggtcctgggctatatgagaagtctacctaagcgtgagcctcagtgaaccagcaagcaccattcccctgcttcaggtctgctgagttcttgctgttggatgtccttcaatgatagactgcaacctggagaggtgcaggccacatagtgatgggctgcgacctagaagtatagccacataaacccctttctccccaggctgcttttgatgagggttttatcacagcaacaccattttaaccctagcctctcctgactgcccagggagtagctgctgcacagagatcttccttctctcttcagctttctcaggccaagcttgtcctatgcggtagagcagtcatgtgcaccccactccaccttccttcagagccgtgctctctataccatttagagccctgtgtagaagataggtaccagaaagattcttgtagctattcagaacatctagtgggagtcccctaaacacccataggctccgttccagcctacagccttccacttattttgagactgcagggttttcttgttttttgtgttttttttgttttttgtttttttttaaaaagcacgtttgatagtctgaggcaaggttttctaatttgttcagatactagttaccttgtgacataattaaataaaagtgatttataattagttcattaaccatgccatttaatgatgctttaaagaagattacccgccctcagagttgtggaagcagctcacttattcttctataggtacttgtgcttgccttgctcagcagctcattccttctgatgaataaagaggcccctacagctcagcacagtgggtcagttgtacttgtgtcaaagaaagctgcaagaccctcctgtcctggctgctctgtgtatgctacttggtcctactaacctgctgctgttagctttaactgttagcttggcatgtgtgtgtgtgtgggggggggggagaattgttttattttctggcaaaaatggttcctgaagtgaagggttttggtctcagaaacttactgattttaaagttttttgagactgggtgtagtgggccacacctttaacgctagcactagggaggcaaaggcaggtgcatctatgtgagttcaagtccagcctggccagcatagcaagtttcaggtcagcacagctatggagtaagatcatgtctgaaagcagcaacagtaaccactgaagaagttggtagaggggaaagaataacttaacagctgaggaactgaggaggtttcagagttttgatcaacagtctggaaagggaaatcagtgcctgaagtcaatgcaggtcctagcatcactttccagtagaagtgggaactgatgggatgaatgggcagctctgcgggtaaagggggtgagggtaacagttacagagccctaggtctgtgggcagcagagaagccacgtgctagcagctcacagttggtgaattacatgctgtggcaacacccagggagggccactggttcttcctgtgcgtgcgttctcacttgtgcgcgtcctccctctccccctctcccccctttttagtttatgtatatgagtgttttgcctgcttgtgtctgtgcaccagctgagatcctggtgctctggggaggttggaaaaatatgttgggtctcctggaattggagtacaaatggttaggagcctccatgtgggtgctaggagcagaacctgggtcctctgaactcctgaaccatgtctctagcaccagacctctttttctttggataaaggtgcgtcatgaagcctaatgtcacatttgggtgtttctctgcctgttgttgtgtggttcttgtgcctccgtcatgcttgctgcaggccaaggctctgtttctgctctgtttctgctctgtttccttgtgctgatgcagtgcagttgctggctgaggttgaaaacagcaccttccaacagtttacagcttgtgcttgtttcgagtggggatcccaagcctgaaggtctgcgatcttgggagtagagaaggagccagctttgcccaggtaggaactggaaggaataggaaccaggaacctaaggtcggggctgcactagccagcttggatttgcttggattacctgggacagcacttcggaagtgaaaaccattctcgcaggatgaccatctctttccttggtagctagatttggtatggagagagttggtttggcttaatagctgggatcctttattcttaagactagcccagagatccaagctccatgtccctgacttgggcattaaagacagaaactgatggaatatacaaaaaataggagaaaatctgtcgtattttcctctggtgttctgagatggtctcttgtggtccaggctggcaccaactccccattctcatgcctccacctcccaagtgctgggttatagacatggactgccacactcctcctgcagttggagacttataggccccagggtatgaaagagtagctctgacacagtgagcatgcaggcctctagcaccctctttgcctgcttccttcatctgcccctattggaggctccaggatctaggaaagtataggagtagggctccctccaggcttccaggccacctgaagcaagaggtttctcagcattgtcccttacctggcaggtaccttacctgcacaggtgtggcttttagtgctaacagcaggcctagtggacagcagcagagtaatttttgactcccacaggcttttgcgtgtgcccagacaggaggtggtgctctgctacctcttcccatggctgctaacagctattgtcaatcatcaagctgcctgtctttgctgactacagtttggtggtgaccctgctttttggggagggccaataggaggcttggctttttttctaaccccctcatctcaaaggcttaaaggcaattcctgtgcagtgtcctgttctgagaacacaaagcacattctttcctgatttttccccccagatcagtttgtaatatttatatcccgtttgtaatgacttcagaacgatgcgtgccttgctgataggaccacagcacagcactggatttaggacacctgtgtttgactgcgcaccacttcttactctgggcacttacaggccatggccttactcctggaggaatgccagccacttccttcttggccccaagtgagtgatcagtatgattgcacactgtgctttactgatggcaggggttttgctgtggatcagaggaccctttccagctttcttctctggagtatcagggaaggccctaagggtaaagtgcacctgagtgaggaagcccagcatgagacccgaggagcgggtgtgtgtgtgtgtgtgtgtgtgtgtgtgtgtgtgtgtgtgtgtgtgtgtgtgtgtgagactgtctcatgctgttggtatttcagatagagcttgaaagcttggccatatgtactgctttgcaagtgaagaccttgagtcatgttttggctattggacctctctgtctacaggatgtttgagtgtggctagtttgagctatgcatgagatttttgtaagctagcctcagtgttctgttgtgaccgggccagttatttaatttttctttttgtctttgaaacctgctgctctgaggctttaaactctgctgttctcatcttgagctcctcccctctggacctagtggacagccagcaggacctcctgtggatttgtatgtctttcctgtctcaggctgtgtgttacctatcatgaggtatgcagtgagctgcctgtgtgactcaggatgtcttggctagggcttgaacctttggctgggtactgtgctgcactgcagttcattgaaaaatgtctgtcgtgagtgtagctttccgactggcattgttttgaatgcatctttaaaggttagttataaatacattaatgcttgggactgacaagaattcatctgtggaactataccatcctgtgagattttatttcattatatatgttcacagaaaagaaacagggagggtagggtgggctggaaagaccctcagcaattaagagcactgcgtgtgctttcagaggacttggggttggttcccagtactcacgtgggagggacctctctactgtgcataactggttccaagggatctgataccctcttatggcctctgagggcactctgtgtgcaaatggtatgcagacatacgtgcaggcaaaactcccatacacttacagtagatactaataaataagctggacttaaattttcattttatgtgtatgaatatttggtatatatcagtgtaatgtgtgtgctatgccttcagaatccagaagagggcattagatcctttagaactagtcttacaaaggcttttgagccacattatggctgctgggaattgaacctgggtcctctggaagaccccagaggagcaagtacttctaataacagagacatcactccagtcccagaagctgggttctgagtggctccaatataatttgaactatagcaaaaggtagaattctgtttaattgtaactctccttcttcttcttcttcttcttcttcttcttcttcttcttcttcttcttcttcttcttcttcttcttctcctcctcctcctcctcctcctcctcctcctcctcctcctcctcctcctcctcctcctcttcttccttcttcttcttctttcttcttctttcttcttcttcttcttttctttttttggttttttgagacagggtttctctgtatagccctggctgtcctggaactcactttgtagaccaggcaggcctcaaactcagaaatccgcctgcctctgccttccaagtgctgggattaaaggcatgcgccaccacgcccagcttcctttttctcttttttccacagacttcatgaggagataatcgacttttataacttcatgtccccttgtcctgaagaagcagccatgagaagggaggtggtgaaacggatcgaaactgtggtgaaagacctctggcccacagctgatgtgagtatcttgttcacgtaccagtccatgaggttgtgtcagctttatccacatgtgcccacgcttcctattcccacagtagagcagctccccaggcctagtcccgggagcatcgcctgggagggaacggcttagacgagcactctcccctctactgcgtctagccctggtgtagtccagcctctggttagcctgtgaaacccataaataggaatatttatattcttatggtccttcattcccctcctctttcattcttgtttaaaaattgcgttaaggttagaaatctccctcctgctccacccacacttgcagtcactgcagttcacagcatggaacatgtttgcgtcattagaaattgcaagtggcagcagagcatggtggcacatgcctttcatcccagccctcaggcagcaaaggctggaagtttgaggctcaagtgggctacgtagggaaactctgtcttaaaacataacagaatacaagtgttcctggtgtagcactcacatgaatgagtgtgtgtctgtggaaatgaggtacttgcctatttgtagtccttggtgcaggcagcctgtggtatgtcagctgaccagggagctaaccgcctggcctggattttgatgtaaataaatgtacaaagagttttctagtttgttgcaccatttcatctgcttcttcccctaatgtccaagctggttttgtttgtcctcgtgtttttcaaggcatggtgggggtctgctggcttgaactctgggacttctctttcaggtgcagatatttggcagctttagtacaggcctctatcttccaacaaggtgagtgtcaaggctgacggcacacttgggtgttggcttatggagtggcagtgtctttgcttctaaggcttgaatagccagctaaggggaaggattgtggaacacaggggaaataggcagaaagttgcatttattagtttagtattaaggcaggagattgcttgcttggcatttttcttcttgcatttaatttctttctttttttttgggggggggtatatttggataatagtttattggttaaaagaatatatttccacaaagatttttttgatttgtttacagagctacccatattttatagaatacctgaagaagctggaaagctgttcagatttcatgtgctaaaaaaagaatattcttaattaaattgtaagaaaaacactaaaatacacaggacaaataagcctcaagagaaaatatggctgaacaaaaataatcagcaactacatcactttataacacaggaattataatgaaatgaagttctctctggacgactccagctatacatgaaatgaaaagtattttcaacatacatccaatttccaaagtacaacataatgaaacatttaaaacttcagtgtattttgattagttcttaaagccccttcccccaaacattttggcaccaaacaaacttttgctacaaatggggatttttttcctttgagatttcctgcatgaccagtgttgataaaataaggaaaggggaaaggctgattgacaagaacagctacataaactattagaagaccattcctaatcgagaaatgaattggtactaaccactgtgtgcatacacttagatcaatgcctgtcagagccttacaacaacgaatggcagtcttaatcaacacagagggatctttttctgggtttggtccatccagcgaaggagaccagtggcctccaatggccatggcttcatccttgcctttcattcccactagaaactaattcaaaccaaagaatcatttatatatatataacacagcccatcaaattataatagatacttagaaaattaggaaggtacaaaccttcccaatactacttttactacctctgatatctgtcagtcaaggacgagttcagttcagcatttaatttcttaagcacagaaatgttgggtgtcagacagaaaatggatgggaactgccttggtctcctgctgccatagaacaggaccccgggttggctgaactgtaaactgtagaagcttcgttagctctcagttctggggaccgagaagtcttaagattcagaggcttatctgggtctaactcaccccgtagcagaggtcttgatccatttagaaaacattgccccagagcctactcacctcaggaaggagaaaggcctgacctgttggttgggggggccacatttcagtgggagtctttggggttttgatagtgaatagaaaatgttagcgaaccttcaagcacacttctgttagaacagtattttatgaagctctttatgtagaagtgtcagatttacatcctaaaattgtcttctgggttcatattgtcatatatataatgtataggtggtttttgttgtacatgctggttagcatttggaactagcctgctagtatagcatttggctgtgatgtcaggacttaggaactgacatgggcaaaggctctggaagccaagtagattaaaagtcttcagggatctccagaccttgaagcaagagccagtgacgatagaatagactgctgaaaacagtttatcctgtcacctgttggttcctgggactctgaggcccttttcactctggctttatatacttagatgactcaagagctgcattcagggctcatggcttaggtacttaggggaccttgttgataagaggcaacatgctgtggtgctggccttcccatgggaggccacctggtgaggtttgttgtgactgacacagtttctcttctgtccagtgacatagacctggttgtctttggaaagtgggaacgccctccattacagttgttggaacaagccctccggaagcacaacgtggctgagccgtgctccatcaaagttcttgacaaagctacagtgagtattgggggacagataccttcacctgaagtccccacatggggtcagtgtcctctggttttcttacagtggttactggttatattttgattgtcctcttgtggtataaccataccagtgaccatcttcatgatttaccagctgagaacctgtttggtgataagtggatttgttctcattgtgagatcttagattttcactcttggccttaaaagacttgtgtctgtctgtgtctgtagagtaaagcaaggcagaggatcactacggaggaactagcactccacctgctgttgtcctggtttgcagcttagagatctgtgtggcagtggtgtgtgtgtgtgtgtgtgtgtgtgtgtgtgtgtgtgtgtgtgtgtgtaagaccgcacactaacttacgctgaaggaggaaaacctggagttccctttgttgctcagccctgtgtcttcttaggaaaggcactgtctcaggggaaactgaaagataagattgcttctaagaagttcctgggacgattcattgaaatggtttatgttagacagcttatgatctcagtctgtggccctggtatgcaccagacaccagctctgctgccatggactggtgtttcagttttgagttggtgtgttacctcgtctgtagggtttttggggaaagtgtgagtttcaatccagtggctgcaagctttgttggtacttttgcctggggtgggtgagtgtttagatggaagcatgcccactgtggattggatcttcacaaagtgtctagactgtcccttcccagtggttgtccctccaggcctggctaccatactgccttttattttattcaccggaactctgcttagtcatgaaccttgggtaagatgcagaaggccatgttgtactcagtcagtcctccaactcaccggcaggcacatgaggaaacacccacaatgaatagcaagggaaaggctttgagctcctttaaacacagtgggcagaggtgacaccttgctatgaccagggtcctgctgtggtccagggtcctgttgtgatccagggaaggtcctgctgtgatccagggaaggtcctgctgtggtctggggtcctgctgtggccccatctgtgccatggttgttctttttgtctttaattcttcatccagggagaaatccagctgtcttaggcactctgaccactcagtcttgcacagttagtgaagtgaggccctttgacaatgtggaggcaatggggacttgctgttggtggtggtattcagatgtaggagagcttatgggtggcctgaggttcctgagttgttgaacttttcaagatccagacagagcatttgaggattgttgggtataactgcttagtcctcccagactccctgctccagcatgttgaaaaatatatacctcaattacttgaaggcatcccatcctggctggtgtgtgtgtgtgtgtgtgtgtgtgtgtgtgtgtgtgtgtgtgtgtgtgtgtgtgtgtgtgtgtgtgtgcgctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttctcttcttctcttcctctccctcctgctcgtcctgtgatgataaccatggatctggatcttgaagtgcaagggctttgtgctgctgtgtatttctcagtgtctggtctaacagagcagctgtggtgctctcacccttgggtgtgtagtgtgactttgtagggtgcatgtgcaggtcaggcctctcacctgagtcctctctcaggctcccattcccctcttgctgtcccacttcagagttggcctgtaggagatggtagagtgggagtcctgctaggagaagacaggtcatggacgttgtgtggagcttcatgaccactgattttaactcctgacagagcccttctctttaaaactgacatccttgaccaccaattcccttttgtgaagagagaacggtgagtgactgtctggagatgtagatgtcacagcagtttgtcaactacatcctcataggtgcccatcataaagctcacagatcaggagactgaagttaaagtcgacatcagctttaacatggagactggcgtgcgggcggcagagttcatcaagaattacatgaaggtactgttgtctcaggtgcccagtaggacagaggtgtgagaccttgttactcatttttctcatgaatgttaaaaaccttaggatgggtcatgggtagcaatacagacagtgcaatttccaggttctgtcatggggtctgtgggggcactcaggaagtgtgtagttgctttctgattggctttgagtgactgcttcttttagagcactctggggaaatgttctgtgttagtgcacagcctcgctcctgctcccggctttgtgctactctcccaacctactttctcacttgccagactccgtctaggttggcttagtttagaggcctcagtgcttgtgaatgtgagagtggctggtgcctgagctgctgctttctggagctttgagtgttggcactcaggccacacagccaagcctgtggtctctttcctctgtcagttccccttgtctgtcattgatctgcaggagcatttgcccacttccctcgctctctgtccctgctttccctagttctgttcagggctgagccacctgcttactgagtgctgggattcttgtgtcccactgatggggtcatggcttgggcctgggggtgtgggggcagtaccctcagctagtgtgaatccttaagcctaaaattggagtcaagagtttttggggggagggagcaggagaatgggtgaggagttctttttctcccaggaagcttgtctagagtcccttcaaaagtacatcagctacggggctggagagagagctcagaggttttaagagttttttcaattcaattcccagcaaccgcccagtagcttatagctatctataatgagatctagtgccctcttctggcctgcaggtgtacatgcaggcataattaaaacaataacaaaaaaaatatcagctgacctgtatgcctcttcttgggtttcagaagtactcgctactgccatacttgattttagtgctgaaacagttcctgctgcagagggacctgaatgaggtcttcacaggcgggatcagctcctacagcctcatcctaatggccatcagctttctgcaggtgagcggcctccgcactgtgctctcctcaccctcactgggctgaggaggcgcccttttctgcttctcctcacaatgctttcttggtcagcccccgtcctgtaggttgattctgtgggtgtaggtttcagaccctgggtgatgcctgcctgtgcttcaggaagctccaacttttactatactgagaactgtggattgtcagtttggagcaggagagcaagaattggtttattcctatataaatcataaaaagccttttccactggcgcttatctgtaaattactcccaaataccctgagagtaaagggtagttaccaacatggggatgagatggagacatttccttttgtgtttttgagataaagtcactctagtcctggatgacctagaactcatgtgcagcccagcctggctttggacccatggcagtcctcttgactcagtttccctggcactgagactgcacatgtgagccactctgcctggcaagccctctgtgattattggtcttaaattcggtgtcatctctgctttgcgagtagagcactaggagctgccctgtcagcagctggcctcccaagcaaggcgctcctctccttgctggttggttggttggtgatgttgagccagaacctcttacatactgacctggaattcactttgtagcccaggctggccttgaactagtagcaagtcttctgtcttaggctcctgagcactgagattatagatgtgagctcttacacctgctaactcatatacaaaatagcacctgtgttaaagttttattgcatttctattggcttgagaaaacatttgcctgtgtgtgttagtcacctgagtgtatgtccccatgtatatcactgagagtgtatgtatgtccccatgtatatcactgagagtgtatgtatgtccccatgtatatcactgagagtgtatgtatgtccccatgtatatcactgagagtgtatgtatgtccccatgtatatcactgagagtgtatgtccccatgtatatcactgagagtgtatgtatgtccccatgtatatcactgagagtgtatacatgtccccacgtatatcactgagagtgtacgtatgtccccatggctgcccaagtctctttccccacgtatatcactggttctcctttgtgagtgcttgctgagggtttgttattcccaatgacactgctagcctgcaagtcagactgtagctcggactgtggtgagctttgatgcagacgtgttgcccctgaagcattagtccccaggtgtgacagcggagaggcttacagtgctgagccatcacagtgggtgacattactgttagcgacatggcctctgtttatatcttgagtacccctgatttgaggtaatgtgtttggatatcttgtcaggacaggtgacgttaccttgagtggggtttcccaaggcttcctggtgtgctgttactaggatccttctggctgtcagacctcacagagctatatagatgggatagccctccttctgtgcctgtgttggagaccacccacctgaggctggacctggtgtagcattctgcttatctcactgctgctgcctgagggaagaaggccaggccctgtactgggctgccttgagtatttgatttggctcaaaagcaaggacataaaggcttggcaagaggactgccctgttatgtagaggagggaagaactctgacattatgagggattattcctggaaggccacattcatgcttcagccctgtgctaccaggacttggacactagggaggcttgaccgtcaccatctagaattgtgcaaaggacaagttctctgtgtgaccatggaagcgatgagccacacgctactcttggatgctgagagtaactggcatgcccacactcattctgtagtgggcagctcaggtctataaagttgctggcacctgggagttagttatcagggaccgtgggcaagcgtcacagagaggctgtggcttgctagaacaaagtgtgcaggtgaaatagcctgacagtgatgtgtgaaagctggaagtgagttcccaggcagagttagctctgtccactcacatcctggtccaatgattgtgatactgttcagtctcatcctgctccctcctggtagcatgtcaggatgatgacattgatgtgaaccatgcagccaccatccagatgacaatgtggaaaacttaccattgcagcatgagggtaactagatgtgtgtgcttggatgtcgtggtctgaatggatgttgagatgtgttctttctttcaagcctcccaccaacagaggctgtccattgtcttgtgttttctcaggtgctatcgagtggggacccctcagtgggttcctggagagctgctaggtgagtgttgttggagtgtagcttgaattcatatgctggtggctctcttgcttttacagttacatccaagaatcgatgcccggagagctgatgaaaacctgggaatgcttcttgtagaattttttgaactttatggaagaaattttaattacttaaaaactggtattagaataaaagaaggaggtgcctatatcgccaaagaagagatcatgaaagccatgaccagtgggtaccgaccatcgatgctgtgcattgaggaccccttactgcctggtgagcttgcccctctggctggaagcacagtgctggcttatctctaccagcagaactcagtgggaacactttcaggtttctaacgagtttactgcactttaaaatgtttacattctggaaatatcataactaggaaaggctaatcagggaatcctcacaggaaacattcacttcaggaaggaagcatgggccaggaagtgaagaatggggactcctgcaggtgattctcctggctttcctatgtcactgcaaatgttgctcttgcagtatgtggtggctgagctaggactccaagccccactccattggcacgtgtaagggtgcttatctcacccaccgtggtaacacttggtctttaagtccttgccaatctgataactgctgcacattcttatttagttatagtccttcctggtgcttgctcttcaactctgcatatgcctgttgagcctgtcttgggtgataggctaagtgcatgatgggatcagggcaggctgtgacaacaggacgacgggaggctgcaaacgccttggctttgttcttctttcgtggaactgtgcgctgcgctgcagccatggtctcctgagctggccacgttggtgttctgtgctaggggagccttgtccacatgactctcagatcccagcttgtgctttcatttgtattcagtcgcttattattctgctcctattccttatttatttactatcctcatttttttaaataatgtttttattaaatattttctttatttacatttcaaatgttatcccctttcctggtttcccctccgaaaaccccctatcctcatccccacttccccctgctcaccaacccacccactcccacttccctgtcctgttattcccctatactgggccttcgagccttcacaagaccaagaggcctctcctctcattgatgtcctacaaggccatcctctgctacatatgtggctggagccatgggtccctccatgtgtactagctggttggtggtttagtccctgggagctctggggggttctggttggttgatattattattccttctatggggctgcaaacctcttcagctccttcagtccattctctagcttctccattggggactctgtgcagtggttggctgtgagcattcacttctgtatttgtcaggttctggcagagcctctcaggagacagctatatcaggctccagtcagcaaacacttgttggcatccacaatagtgtctgggtttgttgtctgtatttgggatagatccccaggtgggacagtctctggatggcctttctttcagtttctgctctatactttgtctccgtatctcctcccatggatattttcttccccctactgtcctcaaatttaatgattatttttaaactgttcgttctagggtgagactaatttctttaaaaatcttttgaaaggaaaatagacaattctagtgagctgtcttcaagtggctaacggatcccataaatggggaggtggggttttcgtggaaacgtcctgacagtgtgtcatctacttgagtgtcccatgtcttcaggttctatttcagtgtgcctgcattactcctggactgtagctgtgtctaggaagatgaaattcccatatcctccctaaaatcccatacttgagatgactgagcagtgaatctatgtaggagttacagttcctcttgatgctagtggtagcttcatttgctttgctctgtgagtttgtaatacagcattcttcccttaaagggcttccttccagttctgatactctgttcagtgtgagcgaccccgttacactgtgtgccactcagggcttatcatgacattgatcatgccaagtccacactgctggttggtgtgttggatattctggatttccttgtggtgtgggcttccctttttttttctttttcccttttctgctacctggggtcttagcagactctgtgaactgtcagacacttgtggctgccccctttccattgttaggaatagactggtcacagagtctgaagcactgaacctgtgtgcaattatataccatgcacttgcagcagctcgctcgggtctgtgtttacatgcctgttgcttgagctgctgtagttgggaggcatttggtggtgagcccctattcacagtaccttgtatgagagacaggtgcctttgtgttgcaggaaatgatgttggacggagttcctatggggccatgcaggtgaagcaggtgtttgactacgcttacattgtgctcagccacgctgtgtcaccgctcgccaggtcttaccccaacagggattctgaaaggtaatgggtcttgtgcctgggttctaggctcctctttcctgtagagtagcaggtgtactttttcatgtgcttctgtggcatggatggatacactgtcatacttgagtggaccttgaggaaagcttttttgggggcaacaagatggcttttgggtagcccgtggcctaggacagcatttcttagtggggtggtattgtacttgggacattctgtattagagcagattcctgactttgaaaacctcggttggtgcctgtccagcagctgtcaggtgtgcttgcttaggttgtgagtgggttcagatctgaacacactgctctgcctctacagctgtttagccgtccttcactttacagttgaggatagtctacagtgtctcctttagtggttgtcaggcatatttatttccagttcagcttagctgctatagtatgtgcacatggggagcaggaggagacaagagtggaaggggagaaatgggctgaacagactgctgtcagcagtgtgtgaatgtctgttccagagctggctgcctcaggaaactgttaggaatgggcctggctttccatgattgtccacacacagtgttgcaggccagcccctagcctgtaatgggcgcctgggtatctctccacacctgtgcagtggaaacaaagtgtcaggacttcatggctaccgcttaaactggattgaagccatcatttgtagacagatcttggttaaggacttctacatagtgaacctacctttatctgtaccgtgatctcatggtactatgaatgtaaacatgtcagacctcatgctctctcctgtctttaatgagcctcacatctgacacctagtgcttatgtccaaccctgttctctgcagtactttaggaagaatcatcaaagtcacccaggaagtgatcgactaccggaggtggatcaaagaaaagtggggtagcagaatcctcccgtctccagaccttggtgagagactgacagtgtgaggcgtggcctctcaccatagcacagaagcatctctgccctcttgtagggttggccaagctgtgaaagtagttccttttctactttttctgataggctatccgttctgtaagctctggaccctatagaaatacagtcgtagctgcagtaaacgaagggaaggagcagatggaagtggttgctaatgttgcatttgattaaacgaaggtatactccaacacagtggcaagaaatcactgagatgcagataggttttgttccaaatcttagaaatccagtatgtatttgtttcagaattctacattctacattgacattggacatatttgttttatgtttatagtttatgacagctacaggttaagacttacacagcactggacaaattatagtgttttctggtgactagtttgattcacatttaaaattagattaactcagatacaaggtttggtttgttaacattcagagttcagcagccttgtgcttcgagcacattgtactaatcactaacctggactttgctttcctgccccagacaacaggattaagataaaggagagaattaccacgtgcaatggggagcagatgcagagccgggagcccagctccccttacacccagcgcctgactctgtccctgtccagtccccagctgctgtcttcaggctcttctgcttcttctgtgtcctcactttctggaagtgacattgtgagtgggcttttgctcttgactcgtccttctcatggggtggggtggccactgtccttgttatctgtagcagggagttcaagacctattcctcctccatctcagtcccttgctggggtgtgaggatgtgtgtgtgtgtgagagatgtatgtgtgtgtgtgagatgtatttgtgtggatatctatggagtatatgtgaggtacgtgtgggtgggaggttggggtgtgtgtgtgtggtgtcgtgtctgtctttgttgggtgttctttgacataggctttaagaacccctggctgtgttgctccgatgacccatcaatagcactcagccaaactttggactttgagtggttttttttgagaaatggtttagagatttaccggttaggtccatgtgcctcatctgcattttctgcagcctagtctttctcgtgctgcagtgactgaggaaggataggtttgctgtaatggcctttcctagcttcctccttaaaggcgcctggctcacacttcagctgcgctgtgcttatgactctccttgtctctgtgctgcgctctaggactccgacacaccgccctgcaccacacccagtgtttatcagttcagcctgcaagcacccactaccctgatggccagcctgcccacagccttgccaatgcccagcagcaaaccccagcctgctgcttccagaacgttgatcatgacaacaaacaaccaggtacaggcttcctccccggggacggcatccgggccaagcaggaccctagatcagcgttcttgtggacgactgggtctagtgctgtctcccggcagtcatcgtgctttggtggctgcagttgttactcagcctgttgtgtaacgcagtctctgacctggagtctgatgttgttctggagcactggatgcctctgacaggtgctctctcagtgccgctttgcatgcagtagtgtccagggcacgcactgctggctgcgtagacacttgctctctctctccagaaactcagtgtcagcatgggcttagactctctgttagtcagtcagtactcgggacttctctttttaaattttatataaagtgaatcaaaagaaaagtatagaaattcagtattgtgttgggtttgtacaaactcagtgcattattaactgggaaggagaagcagcatttgacgtgggagttgagtgggaaacagaacagaagcatgtagatggctttcctggagtcctttggccctgctgagatccattcccacacagctgctttgaagctgagctctacgaaagcatgtgactctgatgttggtgagcgttggacacccttcccccagtgtagcccagactacagacctcaacgagtgcacgcgaggaactttccaaggcagttgttgaagctattaaattctaccccacttcaacttgactgtcagcttccggaaaggacactgcccacccttccactctggaggcctctttgtcactcgccaggacagtgtcttggagagcccgatgcagggccagcttcagtttcctgccggaggtgttttggacctgtggtgtctaagaaagtataggtgtggatcccagttggttgtcacagttgtatgattatcctgtgtgagtgtgagtcaggctgagcttgaaaccaaagtttgctgtaatggcctggagcagagtgctggcatccgattctccaccatgtgctttctacagaggtagccaggctgctgcctctcccagcagctgctatccccctatggagctctgctgtcaaaggttagctcctactgtagcaagtctctctgcaagtgctgggtaaagcagcgatacctgcagtgtcaggctgagagtggcacttaccagctttcttttgctttgggtgggacagataatgcggtggccagaaagagccgccctctttgactcttcttgtgttcctctgttcctctggggtctgagggttgttcactttggggctcattctagggactaaagtgacagttgctgttttggtggctctaattgttttgaatttggtttgatctttggagaatgttagattttatgacctagtgactgttataacttggtattgtaattgctatttgagaagatgagatgattgtaattccaaggccacagtgtccttaggtcacaacagaagctgtgctccgtgggcatgtgtgtgagtggattcgcacagccagccccacactgctttcacttggtgtgaagtttcacttgtgggaagctcatgtttctcttttccttctagaccagggttactatccctccaccaaccctcggagtcgcccctgttccttgcagacaggctggtgtcgacggaaccacatctttgaaggctgttcacagtgtaacttccccagccattccctcggcatcccccaacccactgtctagtcctcatctgtatcacaaggtaagtgtcgtctgctgctgggctactttactgcagccgcttgctcactcggggccctgcaccccttggaactgaattcccctgcatgctctctggttggtggttcagtctctgtgagcccccatgggcccgggtttgttgactctgaccctgcagggaccttctcacacccatgaagaggcagagcagattaaaagcctgtaggctctgagtggagccatagtctgttcagagggaagggttccctcagcttactgcacgccgcagcagggattatgtggctatgggttcctgttgtacattgtgtcttttcacctcatggtctgtgtgctgcactccagccctgctttctctctctccagcagcacaatggcatgaaactgtccatgaaaggctcccacaaccacactcagggtggcggctacagctctgtgggcagtggagctgtgaggcctcctgtaggcaaccgaggacatcatcagtacaaccgcaccggctggaggagaaaaaagcacgcacacacaagggacagcctgcccgtgagtctcagcagataatggtcctgactgactgcccaaaggcctcgctcgggcaccacaggggagccgagaccagcatccagcacctccaccgctgtctgccaagcgcagcccagcactggtcactctgcatgtttgtgtggtggccgcatccatcttacagaacagctccttgtgctcatctgtgaagccttactacatgtggatgtgcgtctgcctgcctagaagtcttcatctgtgcccagcagggcggcctaggagtgtcagggactggatgtgcggctgtgcagccgggtcagctaggtggccatttggggttctcatcgtgtctgtcacaggcagagacgccaagccctgctccgtgtcttctgacggagtgggcaggctgctgttttactgccctcatgtcttgtttgaaaatttgggactgtttttctatgtaaatattgaaaacttatgatttgtgcaataactcagatattttttatttaatttcatattttcacataagttatatttaagggaggagggaattttttttaaaacacgcttaaatcctttcccaatttgcattttctaagttgggttcctcgtgttggctggttatctgatagcatcctgacatgaacaccgtgaggagggaggggcctgtgggctttgtttttatgtcttttcttttggtcagatgcagtgaaggagccaagtcaacttgatagttctgtgaggccagtgtgtacctggcagcctggctgtcggccttgggcctttgtcctctctgaccacgagttctgacattttggtttgggtttttaaaaactagacaccaaatccagcatttaaagtgccagaagtaagaacctctaagaggagaagaggttgtcacgtcgtataaatcgtaaagaatcgtgactctgggcgtgcttcggtcattaagtgacgtggtctagagtcacgggcctggtgagtatcgttacagacaatggcacccagatttaggaatgtggagaaagggattttgttgattccattgaggaatctgcataggtatgcactcgttctgttaagagcaaatatctgaaaaggacctccgttgtccaggcacatgggggtattttaatgtatcacaggagagcacagccccagtgtgggcccaggagcggctggtgcctggcgtcagaagcatacaggtatactatgcaagtgtattctgccacaaccactgtctttgttactttttttgaacaagaatccatccattgcctgaccctgattctcaagcaccacggttgtcctggagtgcttgcagttgtaggccctctgacttctgcttctaaaacgggggtctggacctgctgcacaccacaggcaggttgcttcttgtccaccagtacagagtgtcaagccgagtgctgtgccacctgattgacatgcagcagtggaaattctgaatggatgtctgagtgacattggacacctcgccaaggacaagctctgtgtgtctgggtctgcctcctgctgccgtggtgaggctgttgactctgggaggcattccagagcagaggagcacatgggtctgcagctcatgaggattggagtcatccagaatatttaaaattatttaaattgtgaagcctgttgctaaagaatatttatgaacactggtccatagcctgtactaatttacacattagcaatattgactgtatctgcattaaggagccaccgtggggccgttcgagtgacccgcagatgtgcgttttaaagttctgtcatccacaggcacaggtatgtgtccgtctccgtcatggtgaaccagatgaattggcctggcgaccactgtggccatatgctacagtttacaaaatgataccatgtttaaattttctgtgcggacaacaatgtggacactaaaattaacatatttttatgtaaagtttttctattctttgatctttaataaactttagatgctaga

SEQ ID NO: 7 - DNA, 16 bp, synthetic construct
  misc_feature 1..16: note = oligonucleotide motif
  source 1..16: mol_type = other DNA
agatctgcatccacag

SEQ ID NO: 8 - DNA, 17 bp, synthetic construct
  misc_feature 1..17: note = oligonucleotide motif
  source 1..17: mol_type = other DNA
cagatctgcatccacag

SEQ ID NO: 9 - DNA, 18 bp, synthetic construct
  misc_feature 1..18: note = oligonucleotide motif
  source 1..18: mol_type = other DNA
ccagatctgcatccacag

SEQ ID NO: 10 - DNA, 17 bp, synthetic construct
  misc_feature 1..17: note = oligonucleotide motif
  source 1..17: mol_type = other DNA
ccagatctgcatccaca

SEQ ID NO: 11 - DNA, 17 bp, synthetic construct
  misc_feature 1..17: note = oligonucleotide motif
  source 1..17: mol_type = other DNA
cccagatctgcatccac

SEQ ID NO: 12 - DNA, 16 bp, synthetic construct
  misc_feature 1..16: note = oligonucleotide motif
  source 1..16: mol_type = other DNA
cccagatctgcatcca

SEQ ID NO: 13 - DNA, 17 bp, synthetic construct
  misc_feature 1..17: note = oligonucleotide motif
  source 1..17: mol_type = other DNA
tcccagatctgcatcca

SEQ ID NO: 14 - DNA, 17 bp, synthetic construct
  misc_feature 1..17: note = oligonucleotide motif
  source 1..17: mol_type = other DNA
gtctcccagatctgcat

SEQ ID NO: 15 - DNA, 16 bp, synthetic construct
  misc_feature 1..16: note = oligonucleotide motif
  source 1..16: mol_type = other DNA
tctcccagatctgcat

SEQ ID NO: 16 - DNA, 16 bp, synthetic construct
  misc_feature 1..16: note = oligonucleotide motif
  source 1..16: mol_type = other DNA
gtctcccagatctgca

SEQ ID NO: 17 - DNA, 17 bp, synthetic construct
  misc_feature 1..17: note = oligonucleotide motif
  source 1..17: mol_type = other DNA
tcaactttcacttcagt

SEQ ID NO: 18 - DNA, 16 bp, synthetic construct
  misc_feature 1..16: note = oligonucleotide motif
  source 1..16: mol_type = other DNA
tcaactttcacttcag

SEQ ID NO: 19 - DNA, 16 bp, synthetic construct
  misc_feature 1..16: note = oligonucleotide motif
  source 1..16: mol_type = other DNA
tgtttcaatactaaaa

SEQ ID NO: 20 - DNA, 18 bp, synthetic construct
  misc_feature 1..18: note = oligonucleotide motif
  source 1..18: mol_type = other DNA
catcaactttcacttcag

SEQ ID NO: 21 - DNA, 19 bp, synthetic construct
  misc_feature 1..19: note = oligonucleotide motif
  source 1..19: mol_type = other DNA
catcaactttcacttcagt

SEQ ID NO: 22 - DNA, 20 bp, synthetic construct
  misc_feature 1..20: note = Oligonucleotide motif
  source 1..20: mol_type = other DNA
caacataagtctacacatcc

SEQ ID NO: 23 - DNA, 19 bp, synthetic construct
  misc_feature 1..19: note = Oligonucleotide motif
  source 1..19: mol_type = other DNA
cagttttaccgattcatca

SEQ ID NO: 24 - DNA, 19 bp, synthetic construct
  misc_feature 1..19: note = Primer
  source 1..19: mol_type = other DNA
ctgtgccttgggtggcttt

SEQ ID NO: 25 - DNA, 24 bp, synthetic construct
  misc_feature 1..24: note = Primer
  source 1..24: mol_type = other DNA
aaggaaagaagtcagaaggcaaaa

SEQ ID NO: 26 - DNA, 34 bp, synthetic construct
  misc_feature 1..34: note = Prope
  misc_feature 9..10: note = ZEN - internal quencher
  source 1..34: mol_type = other DNA
agctccaaattctttataagggtcgatgtccatg

SEQ ID NO: 27 - DNA, 16 bp, synthetic construct
  misc_feature 1..16: note = oligonucleotide motif
  source 1..16: mol_type = other DNA
agcgaagtgcacacgg

SEQ ID NO: 28 - DNA, 13 bp, synthetic construct
  misc_feature 1..13: note = oligonucleotide motif
  source 1..13: mol_type = other DNA
gcgtaaagagagg

SEQ ID NO: 29 - DNA, 18 bp, synthetic construct
  misc_feature 1..18: note = oligonucleotide motif
  source 1..18: mol_type = other DNA
caagcgaagtgcacacgg

SEQ ID NO: 30 - DNA, 15 bp, synthetic construct
  misc_feature 1..15: note = oligonucleotide motif
  source 1..15: mol_type = other DNA
cagcgtaaagagagg

SEQ ID NO: 31 - DNA, 18 bp, synthetic construct
  misc_feature 1..18: note = oligonucleotide motif
  source 1..18: mol_type = other DNA
caaaggttgttgtactct

SEQ ID NO: 32 - DNA, 17 bp, synthetic construct
  misc_feature 1..17: note = oligonucleotide motif
  source 1..17: mol_type = other DNA
cagttttatgctaatca

SEQ ID NO: 33 - DNA, 17 bp, synthetic construct
  misc_feature 1..17: note = oligonucleotide motif
  source 1..17: mol_type = other DNA
gtattcttattcttgct

SEQ ID NO: 34 - DNA, 19 bp, synthetic construct
  misc_feature 1..19: note = oligonucleotide motif
  source 1..19: mol_type = other DNA
cattgcttttataatccta

(19) United States
(12) Patent Application Publication
(10) Pub. No.: US 2025/0319114 A1
(43) Pub. Date: Oct. 16, 2025

(54) NUCLEIC ACID MOLECULE FOR REDUCTION OF PAPD5 AND PAPD7 mRNA FOR TREATING HEPATITIS B INFECTION

(71) Applicant: Hoffmann-La Roche Inc., Little Falls, NJ (US)

(72) Inventors: Susanne KAMMLER, Horsholm (DK); Anais LOPEZ, Basel (CH); Henrik MUELLER, Basel (CH); Soren OTTOSEN, Horsholm (DK); Lykke PEDERSEN, Horsholm (DK)

(21) Appl. No.: 19/177,040

(22) Filed: Apr. 11, 2025

Related U.S. Application Data
(63) Continuation of application No. 17/813,576, filed on Jul. 19, 2022, now Pat. No. 12,303,525.
Application No. 17/813,576 is a continuation of application No. 16/661,959, filed on Oct. 23, 2019, now Pat. No. 11,484,546.
Application No. 16/661,959 is a continuation of application No. 16/162,279, filed on Oct. 16, 2018, now Pat. No. 10,953,034.

(30) Foreign Application Priority Data
Oct. 16, 2017 (EP) 17196554.4
Dec. 18, 2017 (EP) 17208056.6

(51) Int. Cl. A61K 31/7105; A61K 31/712; A61P 31/20; C07H 21/02; C12N 15/113

(52) U.S. Cl. CPC A61K 31/7105; A61K 31/712; A61P 31/20; C07H 21/02; C12N 15/113; C12N 15/1137; C12N 2310/11; C12N 2310/315; C12N 2310/3231; C12N 2310/341; C12N 2310/343; C12N 2310/346; C12N 2310/351; C12Y 207/07; C12Y 207/07007

(57) ABSTRACT

The present invention relates to nucleic acid molecules that are complementary to both PAP associated domain containing 5 (PAPD5) and PAP associated domain containing 7 (PAPD7), leading to inhibition of the expression of both PAPD5 and PAPD7 when using a single nucleic acid molecule. The invention also provides for PAPD5 and PAPD7 specific nucleic acid molecules for use in treating and/or preventing a HBV infection, in particular a chronic HBV infection. Also comprised in the present invention is a pharmaceutical composition for use in the treatment and/or prevention of a HBV infection.

### FIELD OF THE INVENTION

The present invention relates to nucleic acid molecules that are complementary to both PAP associated domain containing 5 (PAPD5) and PAP associated domain containing 7 (PAPD7), leading to inhibition of the expression of both PAPD5 and PAPD7 when using a single oligonucleotide. The invention also provides for PAPD5 and PAPD7 specific nucleic acid molecules for use in treating and/or preventing a HBV infection, in particular a chronic HBV infection. Also comprised in the present invention is a pharmaceutical composition for use in the treatment and/or prevention of a HBV infection.

### BACKGROUND

HBV infection remains a major health problem worldwide which concerns an estimated 350 million chronic carriers. Approximately 25% of carriers die from chronic hepatitis, cirrhosis, or liver cancer. Hepatitis B virus is the second most significant carcinogen behind tobacco, causing from 60% to 80% of all primary liver cancer. HBV is 100 times more contagious than HIV.

The hepatitis B virus (HBV) is an enveloped, partially double-stranded DNA virus. The compact 3.2 kb HBV genome consists of four overlapping open reading frames (ORF), which encode for the core, polymerase (Pol), envelope and X-proteins. The Pol ORF is the longest and the envelope ORF is located within it, while the X and core ORFs overlap with the Pol ORF. The lifecycle of HBV has two main events: 1) generation of closed circular DNA (cccDNA) from relaxed circular (RC DNA), and 2) reverse transcription of pregenomic RNA (pgRNA) to produce RC DNA. Prior to the infection of host cells, the HBV genome exists within the virion as RC DNA. It has been determined that HBV virions are able to gain entry into host cells by non-specifically binding to the negatively charged proteoglycans present on the surface of human hepatocytes (Schulze, Hepatology, 46, (2007), 1759-68) and via the specific binding of HBV surface antigens (HBsAg) to the hepatocyte sodium-taurocholate cotransporting polypeptide (NTCP) receptor (Yan, J Virol, 87, (2013), 7977-91). All HBV viral mRNAs are capped and polyadenylated, and then exported to the cytoplasm for translation. In the cytoplasm, the assembly of new virons is initiated and nascent pgRNA is packaged with viral Pol so that reverse transcription of pgRNA, via a single stranded DNA intermediate, into RC DNA can commence.

The secretion of antiviral cytokines in response to a HBV infection by the hepatocytes and/or the intra-hepatic immune cells plays a central role in the viral clearance of the infected liver. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signalling system and subsequently the interferon (IFN) antiviral activity. Among these, the excessive secretion of HBV empty sub-viral particles (SVPs, HBsAg) are thought to participate to the maintenance of the immunological tolerant state observed in chronically infected patients (CHB). The persistent exposure to HBsAg and other viral antigens can lead to HBV-specific T-cell deletion or to progressive functional impairment (Kondo, Journal of Immunology (1993), 150, 4659-4671; Kondo, Journal of Medical Virology (2004), 74, 425-433; Fisicaro, Gastroenterology, (2010), 138, 682-93;). Moreover HBsAg has been reported to suppress the function of immune cells such as monocytes, dendritic cells (DCs) and natural killer (NK) cells by direct interaction (Op den Brouw, Immunology, (2009b), 126, 280-9; Woltman, PLoS One, (2011), 6, e15324; Shi, J Viral Hepat. (2012), 19, e26-33; Kondo, ISRN Gasteroenterology, (2013), Article ID 935295).

HBsAg quantification is a significant biomarker for prognosis and treatment response in chronic hepatitis B. However the achievement of HBsAg loss and seroconversion is rarely observed in chronically infected patients but remains one of the ultimate goals of therapy. Current therapy such as Nucleos(t)ide analogues are molecules that inhibit HBV DNA synthesis but are not directed at reducing HBsAg level. Nucleos(t)ide analogs, even with prolonged therapy, only show weak HBsAg clearance comparable to those observed naturally (between −1%-2%) (Janssen, Lancet, (2005), 365, 123-9; Marcellin, N. Engl. J. Med., (2004), 351, 1206-17; Buster, Hepatology, (2007), 46, 388-94). It was recently shown that completely or patially integrated hepatitis B virus DNA is a source of HBsAg expression in chronically infected individuals (see Wooddell et all 2017 Sci. Transl. Med. Vol 9, Issue 409, eaan0241).

Hepatitis B e-antigen (also called HBV envelope antigen or HBeAg) is a viral protein that is secreted by hepatitis B infected cells. HBeAg is associated with chronic hepatitis B infections and is used as a marker of active viral disease and a patient's degree of infectiousness.

The function of the hepatitis B virus precore or HBeAg is not completely known. However HBeAg is well known to play a key role in viral persistence. HBeAg is thought to promote HBV chronicity by functioning as an immunoregulatory protein. In particular, the HBeAg is a secreted accessory protein, which appears to attenuate the host immune response to the intracellular nucleocapsid protein (Walsh, Virology, 2011, 411(1):132-141). The HBeAg acts as an immune tolerogen contributing to HBV persistence, and possibly functions in utero considering that soluble HBeAg traverses the placenta (Walsh, Virology, 2011, 411(1):132-141). Furthermore, HBeAg downregulates: i) cellular genes controlling intracellular signaling; and ii) the Toll-like receptor 2 (TLR-2) to dampen the innate immune response to viral infection (Walsh, Virology, 2011, 411(1):132-141). In the absence of HBeAg, HBV replication is associated with upregulation of the TLR2 pathway (Walsh, Virology, 2011, 411(1):132-141). Accordingly, HBeAg has a significant role in modulating virus/host interactions to influence the host immune response (Walsh, Virology, 2011, 411(1):132-141). Thus, reducing HBeAg in HBeAg positive patient population may lead to reversal of HBV specific immunedys function (Milich, 1997, J. Viral. Hep. 4: 48-59; Milich, 1998, J. Immunol. 160: 2013-2021). In addition, the secreted HBeAg is significantly more efficient than the intracellular hepatitis core antigen (HBcAg) at eliciting T-cell tolerance, and the split T-cell tolerance between the HBeAg and the HBcAg and the clonal heterogeneity of HBc/HBeAg-specific T-cell tolerance may have significant implications for natural HBV infection and especially for precore-negative chronic hepatitis (Chen, 2005, Journal of Virology, 79: 3016-3027).

Accordingly, reducing secretion of HBeAg in addition to secretion of HBsAg would lead to an improved inhibition of development of a chronic HBV infection as compared to the inhibition of secretion of HBsAg alone. In addition, the highest rates of transmission of an acute infection to chronic (>80%) have been reported in cases of materno-fetal and neonatal HBV transmission from HBeAg-positive mothers (Liaw, Lancet, 2009, 373: 582-592; Liaw, Dig. Dis. Sci., 2010, 55: 2727-2734; and Hadziyannis, 2011, Journal of hepatology, 55: 183-191). Therefore, reducing HBeAg in an expected mother may not only reduce the patient's degree of infectiousness, but may also inhibit the development of a chronic HBV infection of her child.

Therefore, in the therapy of HBV there is an unmet medical need to inhibit viral expression, particularly to inhibit secretion of HBsAg and HBeAg (Wieland, S. F. & F. V. Chisari. J Virol, (2005), 79, 9369-80; Kumar et al. J Virol, (2011), 85, 987-95; Woltman et al. PLoS One, (2011), 6, e15324; Op den Brouw et al. Immunology, (2009b), 126, 280-9).

In WO 2017/066712 down regulation of PAPD5 in relation to the treatment and diagnosis of telomere diseases has been described. Five shRNA structures for this purpose have been described.

PCT/EP2017/064980 discloses targeting PAPD5 or PAPD7 with a nucleic acid molecule and the combination of such molecules to treatment HBV infections.

### Objective of the Invention

The present invention identifies novel nucleic acid molecules which are capable of inhibiting the expression of both PAPD5 and PAPD7 in vivo and in vitro. The ability to inhibit two target nucleic acids with a single molecule has distinct advantages in terms of production, simplicity of delivery to the target cell, simplicity of pharmacokinetic/pharmacodynamic (PK/PD) and the concentration needed to achieve a therapeutic benefit. Furthermore the present invention shows that there is a correlation between the PAPD5 and PAPD7 knock down and the HBV antigen inhibition, such as HBsAg inhibition.

### BRIEF DESCRIPTION OF THE FIGURES

The Figures show:

FIG. 1: Illustrates exemplary antisense oligonucleotide conjugates, where the oligonucleotide either is represented as a wavy line (A-D) or as “oligonucleotide” (E-H) or as T₂ (I) and the asialoglycoprotein receptor targeting conjugate moieties are trivalent N-acetylgalactosamine moieties. Compounds A to D comprise a di-lysine brancher molecule, a PEG3 spacer and three terminal GalNAc carbohydrate moieties. In compound A and B the oligonucleotide is attached directly to the asialoglycoprotein receptor targeting conjugate moiety without a linker. In compound C and D the oligonucleotide is attached to the asialoglycoprotein receptor targeting conjugate moiety via a C6 linker. Compounds E-I comprise a commercially available trebler brancher molecule and spacers of varying length and structure and three terminal GalNAc carbohydrate moieties.

FIG. 2: Structural formula of the trivalent GalNAc cluster (GN2). GN2 is useful as conjugation moiety in the present invention. The wavy line illustrates the site of conjugation of the cluster to e.g. a C6 amino linker or directly to the oligonucleotide

FIG. 3: Shows the correlation between PAPD5 and PAPD7 knock down in Hela cells from example 1 with HBsAg reduction in dHepRG cells from example 2.

FIG. 4: Structural formula of CMP ID NO: 20_12. Pharmaceutical salts thereof include monovalent or divalent cations, such as Na⁺, K⁺, and Ca²⁺ or a mixture of these being associated with the compound.

FIG. 5: Structural formula of CMP ID NO: 20_13. Pharmaceutical salts thereof include monovalent or divalent cations, such as Na⁺, K⁺, and Ca²⁺ or a mixture of these being associated with the compound.

FIG. 6: Structural formula of CMP ID NO: 20_14. Pharmaceutical salts thereof include monovalent or divalent cations, such as Na⁺, K⁺, and Ca²⁺ or a mixture of these being associated with the compound.

FIG. 7: Structural formula of CMP ID NO: 20_15. Pharmaceutical salts thereof include monovalent or divalent cations, such as Na⁺, K⁺, and Ca²⁺ or a mixture of these being associated with the compound.

FIG. 8: Structural formula of CMP ID NO: 20_18. Pharmaceutical salts thereof include monovalent or divalent cations, such as Na⁺, K⁺, and Ca²⁺ or a mixture of these being associated with the compound.

FIG. 9: Structural formula of CMP ID NO: 20_36. Pharmaceutical salts thereof include monovalent or divalent cations, such as Na⁺, K⁺, and Ca²⁺ or a mixture of these being associated with the compound.

FIG. 10: Structural formula of CMP ID NO: 20_30. Pharmaceutical salts thereof include monovalent or divalent cations, such as Na⁺, K⁺, and Ca²⁺ or a mixture of these being associated with the compound.

FIG. 11: Representation of in vitro PAPD5 and PAPD7 reduction achieved with oligonucleotides targeting the human and mouse transcripts (table 5) in the human HeLa cell line (A) and in primary mouse hepatocytes (PMH, B).

FIG. 12: Structural formula of CMP ID NO: 20_20. Pharmaceutical salts thereof include monovalent or divalent cations, such as Na⁺, K⁺, and Ca²⁺ or a mixture of these being associated with the compound.

FIG. 13: Structural formula of CMP ID NO: 20_21. Pharmaceutical salts thereof include monovalent or divalent cations, such as Na⁺, K⁺, and Ca²⁺ or a mixture of these being associated with the compound.

FIG. 14: Structural formula of CMP ID NO: 21_2. Pharmaceutical salts thereof include monovalent or divalent cations, such as Na⁺, K⁺, and Ca²⁺ or a mixture of these being associated with the compound.

FIG. 15: Structural formula of CMP ID NO: 20_22. Pharmaceutical salts thereof include monovalent or divalent cations, such as Na⁺, K⁺, and Ca²⁺ or a mixture of these being associated with the compound.

FIG. 16: Structural formula of CMP ID NO: 21_33. Pharmaceutical salts thereof include monovalent or divalent cations, such as Na⁺, K⁺, and Ca²⁺ or a mixture of these being associated with the compound.

FIG. 17: Structural formula of CMP ID NO: 21_34. Pharmaceutical salts thereof include monovalent or divalent cations, such as Na⁺, K⁺, and Ca²⁺ or a mixture of these being associated with the compound.

FIG. 18: Effect on HBsAg and HBeAg over time in vivo in the AAV/HBV mouse model following a single treatment with 10 mg/kg of two oligonucleotides one targeting PAPD5 and one targeting PAPD7.

### SUMMARY OF THE INVENTION

### Definitions

### Nucleic Acid Molecule

The term “nucleic acid molecule” or “therapeutic nucleic acid molecule” as used herein is defined as it is generally understood by the skilled person as a molecule comprising two or more covalently linked nucleosides (i.e. a nucleotide sequence). The nucleic acid molecule(s) referred to in the method of the invention are generally therapeutic oligonucleotides below 50 nucleotides in length. The nucleic acid molecules may be or comprise an antisense oligonucleotide, or may be another oligomeric nucleic acid molecule, such as a CRISPR RNA, a siRNA, shRNA, an aptamer, or a ribozyme. Nucleic acid molecules are compositions that are commonly made in the laboratory by solid-phase chemical synthesis followed by purification. When referring to a sequence of the nucleic acid molecule, reference is made to the sequence or order of nucleobase moieties, or modifications thereof, of the covalently linked nucleotides or nucleosides. The nucleic acid molecule of the invention is man-made, and is chemically synthesized, and is typically purified or isolated. The nucleic acid molecule of the invention may comprise one or more modified nucleosides or nucleotides.

In some embodiments, the nucleic acid molecule of the invention comprises or consists of 12 to 50 nucleotides in length, such as from 13 to 40, such as from 14 to 35, such as from 15 to 30, such as from 16 to 22, such as from 16 to 18 or 15 to 17 contiguous nucleotides in length.

In some embodiments, the nucleic acid molecule or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 10 to 30 nucleotides, both 10 and 30 nucleotides are included.

In some embodiments, the contiguous nucleotide sequence comprises or consists of 12, 13, 14, 15, 16, 17, 18, 19, 20, 21 or 22 contiguous nucleotides in length

The nucleic acid molecule(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s).

In one embodiment of the invention the nucleic acid molecule is selected from a RNAi agent, such as a siRNA or shRNA. In another embodiment the nucleic acid molecule is a single stranded antisense oligonucleotide, such as a high affinity modified antisense oligonucleotide.

In some embodiments the nucleic acid molecule is a phosphorothioate nucleic acid molecule. In some embodiments the nucleic acid molecule comprises phosphorothioate internucleoside linkages.

In some embodiments the nucleic acid molecule may be conjugated to non-nucleosidic moieties (conjugate moieties).

A library of nucleic acid molecules is to be understood as a collection of variant nucleic acid molecules. The purpose of the library of nucleic acid molecules can vary. In some embodiments, the library of nucleic acid molecules is composed of oligonucleotides with overlapping nucleobase sequence targeting a region in common between the PAPD5 and PAPD7 target nucleic acids with the purpose of identifying the most potent sequence within the library of nucleic acid molecules. In some embodiments, the library of nucleic acid molecules is a library of nucleic acid molecule design variants (child nucleic acid molecules) of a parent or ancestral nucleic acid molecule, wherein the nucleic acid molecule design variants retaining the core nucleobase sequence of the parent nucleic acid molecule.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. The term single stranded is generally understood by the skilled person in the art. Especially it is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

In one embodiment of the invention the antisense oligonucleotide is an RNaseH recruiting oligonucleotide. Contrary to RNAi molecules antisense oligonucleotides also act in the nucleous of the cell. For targeting pre-mRNA sequences and antisense oligonucleotide is preferable since it acts in the nucleus of the cell.

### RNAi

Herein, the term “RNA interference (RNAi) molecule” refers to short double-stranded RNA molecule capable of inducing RNA-dependent gene silencing via the RNA-induced silencing complex (RISC) in a cell's cytoplasm, where they interact with the catalytic RISC component argonaute. One type of RNAi molecule is a small interfering RNA (siRNA), which is a double-stranded RNA molecule that, by binding complementary mRNA after transcription, leads to their degradation and loss in translation. A small hairpin RNA (shRNA) is an artificial RNA molecule with a hairpin structure which upon expression is able to reduce mRNA via the DICER and RNA reducing silencing complex (RISC). RNAi molecules can be designed on the base of the RNA sequence of the gene of interest. Corresponding RNAi can then be synthesized chemically or by in vitro transcription, or expressed from a vector or PCR product

siRNA and shRNA molecules are generally between 20 and 50 nucleotides in length, such as between 25 and 35 nucleotides in length, and interacts with the endonuclease known as Dicer which is believed to processes dsRNA into 19-23 base pair short interfering RNAs with characteristic two base 3′ overhangs which are then incorporated into an RNA-induced silencing complex (RISC). Effective extended forms of Dicer substrates have been described in U.S. Pat. Nos. 8,349,809 and 8,513,207, hereby incorporated by reference. Upon binding to the appropriate target mRNA, one or more endonucleases within the RISC cleave the target to induce silencing. RNAi agents may be chemically modified using modified internucleotide linkages and high affinity nucleosides, such as 2′-4′ bicyclic ribose modified nucleosides, including LNA and cET.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence and may optionally comprise further nucleotide(s), for example a nucleotide linker region which may be used to attach a functional group to the contiguous nucleotide sequence. The nucleotide linker region may or may not be complementary to the target nucleic acid.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. Nucleotides with modified internucleoside linkage are also termed “modified nucleotides”. In some embodiments, the modified internucleoside linkage increases the nuclease resistance of the nucleic acid molecules of the invention compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides as well as siRNA's for in vivo use, and may serve to protect against nuclease cleavage at regions of DNA or RNA nucleosides in the oligonucleotide or siRNA of the invention, for example within the gap region of a gapmer oligonucleotide, as well as in regions of modified nucleosides.

In an embodiment, the nucleic acid molecule, e.g. antisense oligonucleotide, shRNA or siRNA, comprises one or more internucleoside linkages modified from the natural phosphodiester to a linkage that is for example more resistant to nuclease attack. Nuclease resistance may be determined by incubating the oligonucleotide in blood serum or by using a nuclease resistance assay (e.g. snake venom phosphodiesterase (SVPD), both are well known in the art. Internucleoside linkages which are capable of enhancing the nuclease resistance of an oligonucleotide are referred to as nuclease resistant internucleoside linkages. In some embodiments at least 50% of the internucleoside linkages in the antisense oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

Modified internucleoside linkages may be selected from the group comprising phosphorothioate, diphosphorothioate and boranophosphate. In some embodiments, the modified internucleoside linkages are compatible with the RNaseH recruitment of the oligonucleotide of the invention, for example phosphorothioate, diphosphorothioate or boranophosphate.

In some embodiments the internucleoside linkage comprises sulphur (S), such as a phosphorothioate internucleoside linkage.

A phosphorothioate internucleoside linkage is particularly useful due to nuclease resistance, beneficial pharmakokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate. In some embodiments at least one of the phosphorothioate internucleoside linkages is stereodefined, such as at least 20%, 30%, 40%, 50%, 60%, such as at least 70%, such as at least 75%, such as at least 80% or such as at least 90% of the internucleoside linkages in the oligonucleotide are stereo defined. The synthesis of stereodefined phosphorothiate linkages are for example described in WO2014/012081 and WO2016/079181.

In some embodiments, the oligonucleotide comprises one or more neutral internucleoside linkage, particularly a internucleoside linkage selected from phosphotriester, methylphosphonate, MMI, amide-3, formacetal or thioformacetal.

Further internucleoside linkages are disclosed in WO2009/124238 (incorporated herein by reference). In an embodiment the internucleoside linkage is selected from linkers disclosed in WO2007/031091 (incorporated herein by reference). Particularly, the internucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, and/or the internucleoside linker may be selected form the group consisting of: —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is selected from hydrogen and C1-4-alkyl.

Nuclease resistant linkages, such as phosphothioate linkages, are particularly useful in antisense oligonucleotide regions capable of recruiting nuclease when forming a duplex with the target nucleic acid, such as region G for gapmers, or the non-modified nucleoside region of headmers and tailmers. Phosphorothioate linkages may, however, also be useful in non-nuclease recruiting regions and/or affinity enhancing regions such as regions F and F′ for gapmers, or the modified nucleoside region of headmers and tailmers.

Each of the design regions may however comprise internucleoside linkages other than phosphorothioate, such as phosphodiester linkages, in particularly in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide—see WO2008/113832, incorporated herein by reference.

In an embodiment all the internucleoside linkages in the antisense oligonucleotide are phosphorothioate and/or boranophosphate linkages. Preferably, all the internucleoside linkages in the oligonucleotide are phosphorothioate linkages.

### Stereorandom Phosphorothioate Linkages

Phosphorothioate linkages are internucleoside phosphate linkages where one of the non-bridging oxygens has been substituted with a sulfur. The substitution of one of the non-bridging oxygens with a sulfur introduces a chiral center, and as such within a single phosphorothioate oligonucleotide, each phosphorothioate internucleoside linkage will be either in the S (Sp) or R (Rp) stereoisoforms. Such internucleoside linkages are referred to as “chiral internucleoside linkages”. By comparison, phosphodiester internucleoside linkages are non-chiral as they have two non-terminal oxygen atoms.

The designation of the chirality of a stereocenter is determined by standard Cahn-Ingold-Prelog rules (CIP priority rules) first published in Cahn, R. S.; Ingold, C. K.; Prelog, V. (1966). “Specification of Molecular Chirality”. Angewandte Chemie International Edition. 5 (4): 385-415. doi:10.1002/anie.196603851.

During standard oligonucleotide synthesis the stereoselectivity of the coupling and the following sulfurization is not controlled. For this reason the stereochemistry of each phosphorothioate internucleoside linkages is randomly Sp or Rp, and as such a phosphorothioate oligonucleotide produced by traditional oligonucleotide synthesis actually can exist in as many as 2^{X} different phosphorothioate diastereoisomers, where X is the number of phosphorothioate internucleoside linkages. Such oligonucleotides are referred to as stereorandom phosphorothioate oligonucleotides herein, and do not contain any stereodefined internucleoside linkages. Stereorandom phosphorothioate oligonucleotides are therefore mixtures of individual diastereoisomers originating from the non-stereodefined synthesis. In this context the mixture is defined as up to 2^{X} different phosphorothioate diastereoisomers.

### Stereodefined Internucleoside Linkages

A stereodefined internucleoside linkage is an internucleoside linkage which introduces a chiral center into the oligonucleotide, which exists in predominantly one stereoisomeric form, either R or S within a population of individual oligonucleotide molecules.

It should be recognized that stereoselective oligonucleotide synthesis methods used in the art typically provide at least about 90% or at least about 95% stereoselectivity at each internucleoside linkage stereocenter, and as such up to about 10%, such as about 5% of oligonucleotide molecules may have the alternative stereo isomeric form.

In some embodiments the stereoselectivity of each stereodefined phosphorothioate stereocenter is at least about 90%. In some embodiments the stereoselectivity of each stereodefined phosphorothioate stereocenter is at least about 95%.

### Stereodefined Phosphorothioate Linkages

Stereodefined phosphorothioate linkages are phosphorothioate linkages which have been chemically synthesized in either the Rp or Sp configuration within a population of individual oligonucleotide molecules, such as at least about 90% or at least about 95% stereoselectivity at each stereocenter (either Rp or Sp), and as such up to about 10%, such as about 5% of oligonucleotide molecules may have the alternative stereo isomeric form.

The stereo configurations of the phosphorothioate internucleoside linkages are presented below

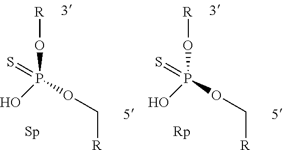

Where the 3′ R group represents the 3′ position of the adjacent nucleoside (a 5′ nucleoside), and the 5′ R group represents the 5′ position of the adjacent nucleoside (a 3′ nucleoside).

Rp internucleoside linkages may also be represented as srP, and Sp internucleoside linkages may be represented as ssP herein.

In some embodiments the stereoselectivity of each stereodefined phosphorothioate stereocenter is at least about 97%. In some embodiments the stereoselectivity of each stereodefined phosphorothioate stereocenter is at least about 98%. In some embodiments the stereoselectivity of each stereodefined phosphorothioate stereocenter is at least about 99%.

In some embodiments a stereoselective internucleoside linkage is in the same stereoisomeric form in at least 97%, such as at least 98%, such as at least 99%, or (essentially) all of the oligonucleotide molecules present in a population of the oligonucleotide molecule.

Stereoselectivity can be measured in a model system only having an achiral backbone (i.e. phosphodiesters) it is possible to measure the stereoselectivity of each monomer by e.g. coupling a stereodefined monomer to the following model-system “5′ t-po-t-po-t-po 3″”. The result of this will then give: 5′ DMTr-t-srp-t-po-t-po-t-po 3′ or 5′ DMTr-t-ssp-t-po-t-po-t-po 3′ which can be separated using HPLC. The stereoselectivity is determined by integrating the UV signal from the two possible compounds and giving a ratio of these e.g. 98:2, 99:1 or >99:1.

It will be understood that the stereo % purity of a specific single diastereoisomer (a single stereodefined oligonucleotide molecule) will be a function of the coupling selectivity for the defined stereocenter at each internucleoside position, and the number of stereodefined internucleoside linkages to be introduced. By way of example, if the coupling selectivity at each position is 97%, the resulting purity of the stereodefined oligonucleotide with 15 stereodefined internucleoside linkages will be 0.97¹⁵, i.e. 63% of the desired diastereoisomer as compared to 37% of the other diastereoisomers. The purity of the defined diastereoisomer may after synthesis be improved by purification, for example by HPLC, such as ion exchange chromatography or reverse phase chromatography.

In some embodiments, a stereodefined oligonucleotide refers to a population of an oligonucleotide wherein at least about 40%, such as at least about 50% of the population is of the desired diastereoisomer.

Alternatively stated, in some embodiments, a stereodefined oligonucleotide refers to a population of oligonucleotides wherein at least about 40%, such as at least about 50%, of the population consists of the desired (specific) stereodefined internucleoside linkage motif (also termed stereodefined motif).

For stereodefined oligonucleotides which comprise both stereorandom and stereodefined internucleoside stereocenters, the purity of the stereodefined oligonucleotide is determined with reference to the % of the population of the oligonucleotide which retains the defined stereodefined internucleoside linkage motif(s), the stereorandom linkages are disregarded in the calculation.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide or modified nucleic acid molecule describes an oligonucleotide or nucleic acid molecule comprising one or more sugar-modified nucleosides and/or modified internucleoside linkages. The term “chimeric” is a term that has been used in the literature to describe oligonucleotides or nucleic acid molecules with modified nucleosides, in particular gapmer oligonucleotides.

### Stereodefined Oligonucleotide

A stereodefined oligonucleotide is an oligonucleotide wherein at least one of the internucleoside linkages is a stereodefined internucleoside linkage.

A stereodefined phosphorothioate oligonucleotide is an oligonucleotide wherein at least one of the internucleoside linkages is a stereodefined phosphorothioate internucleoside linkage.

### Stereodefined Internucleoside Motif

A stereodefined internucleoside motif, also termed stereodefined motif herein, refers to the pattern of stereodefined R and S internucleoside linkages in a stereodefined oligonucleotide, and is written 5′-3′. For example, the stereodefined oligonucleotide

(SEQ ID NO 18)
5′-T_{srP} C_{ssP} A_{ssP} a_{srP} c_{srP} t_{ssP} t_{srP} t_{srP} c_{ssP} a_{srP} c_{ssP}
t_{srP} t_{ssP} C_{ssP} A_{ssP} G-3′,

has a stereodefined internucleoside motif of RSSRRSRRSRSRSSS.

With respect to sub-libraries of stereodefined oligonucleotides, these will contain a common stereodefined internucleoside motif in an otherwise stereorandom background (optionally with one or more non chiral internucleoside linkages, e.g. phosphodiester linkages).

For example, the oligonucleotide

(SEQ ID NO 18)
5′-Tₛ Cₛ Aₛ aₛ c_{srP} t_{ssP} t_{ssP} t_{srP} cₛ aₛ cₛ tₛ tₛ
Cₛ Aₛ G-3

has a stereodefined internucleoside motif of XXXXRSSRXXXXXXX, with X representing a stereorandom phosphorothioate internucleoside linkage (shown as subscript s in the compound). It will be noted that in this example the first 5′ stereodefined internucleoside linkage is the 5^{th} internucleoside linkage from the 5′ end (between the nucleosides at position 4 and 5), and as such the above motif is also referred to as a “RSSR” motif at (internucleoside linkage) position 5.

When the stereodefined internucleoside motif (stereodefined motif) is made up on a series of adjacent stereodefined internucleoside linkages (i.e. positioned between contiguous nucleosides), it is referred to herein as a contiguous stereodefined internucleoside motif (a contiguous stereodefined motif). It will be understood that a contiguous stereodefined motif must comprise two or more adjacent stereodefined internucleoside linkages.

In a sub-library mixture, a stereodefined internucleoside motif may also be dis-contiguous, i.e. the stereodefined internucleoside linkages are dispersed with one or more stereorandom internucleoside linkages.

For example the compound

(SEQ ID NO 18)
5′-Tₛ C_{ssP} Aₛ aₛ c_{srP} t_{ssP} tₛ tₛ cₛ aₛ cₛ tₛ t_{ssP} C_{srP}
A_{ssP} G-3

has a dis-contiguous motif XSXXRSXXXXXXSRS.

### Parent Oligonucleotide

A parent oligonucleotide is an oligonucleotide which has a defined nucleobase sequence (motif sequence). In the methods of the invention, a parent oligonucleotide is typically an oligonucleotide which is to be improved by the use of the method of the invention by creating one or more libraries.

Typically a library can vary the nucleoside modifications (design libraries) while maintaining the nucleobase sequence of the parent and the stereochemistry (typically stereorandom).

Alternative a library can vary the stereochemistry of the parent oligonucleotide while maintaining the nucleobase sequence (motif sequence) and nucleoside modification pattern (design). In such a library the stereochemistry of one, or more (2+), of the internucleoside linkages is stereodefined and is different to that of the parent oligonucleotide.

In some embodiments, the parent oligonucleotide is a stereorandom phosphorothioate oligonucleotide. In some embodiments, the parent oligonucleotide is a stereorandom phosphorothioate oligonucleotide gapmer.

In some embodiments, the parent oligonucleotide may be a sub-library which comprises a common stereodefined motif.

### Stereodefined Variants (Child Oligonucleotides)

A stereodefined variant of an oligonucleotide is an oligonucleotide which retain the same sequence and nucleoside modifications as a parent oligonucleotide (i.e. the same sequence and nucleoside modification chemistry and design), but differs with respect to one or more stereodefined internucleoside linkages, such as one or more stereodefined phosphorothioate internucleoside linkages (a stereodefined phosphorothioate variant).

A stereodefined variant may be a sub-library, or may be a fully stereodefined oligonucleotide.

### Sub-Library of Stereodefined Oligonucleotides

An oligonucleotide which comprises both stereorandom and stereodefined oligonucleotides is referred to herein as a sub-library. Sub-libraries are less complex mixtures of the diastereoisomeric mixture of a fully stereorandom oligonucleotide thus representing a sub-set of all possible diastereoisomers. For example, theoretically, a fully phosphorothioate stereorandom 16mer is a mixture of 2¹⁵ diastereoisomer (32768), whereas a sub-library where one of the phosphorothioate internucleoside linkages is stereodefined will have half the library complexity (16384 diastereoisomer), (2 stereodefined linkages=8192 diastereoisomer; 3 stereodefined linkages=4096 diastereoisomer, 4 stereodefined linkages=2048 diastereoisomer, 5 stereodefined linkages=1024 diastereoisomer) assuming 100% stereoselective coupling efficacy.

### Fully Stereodefined Oligonucleotides

A fully stereodefined oligonucleotide is an oligonucleotide wherein all the chiral internucleoside linkages present within the oligonucleotide are stereodefined. A fully stereodefined phosphorothioate oligonucleotide is an oligonucleotides wherein all the chiral internucleoside linkages present within the oligonucleotide are stereodefined phosphorothioate internucleoside linkages.

It will be understood that, in some embodiments, a fully stereodefined oligonucleotide may comprise one or more, non-chiral internucleosides, such as phosphodiester internucleoside linkages, for example phosphodiester linkages can be used within the flanking regions of gapmers, and/or when linking terminal nucleosides, such as between short regions of DNA nucleosides (biocleavable linker) linking a gapmer sequence and a conjugate group.

In some embodiments of fully stereodefined oligonucleotide, all of the internucleoside linkages present in the oligonucleotide, or contiguous nucleotide region thereof, such as an F-G-F′ gapmer, are stereodefined internucleoside linkages, such as stereodefined phosphorothioate internucleoside linkages.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol. 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

The following is an example of an oligonucleotide (SEQ ID NO: 12) that is fully complementary to a region of a target nucleic acid.

| | | | |
|---|---|---|---|
| 759 | ctgtggatgcagatctgggaga | 781 | (Pos. 759-781 of SEQ ID NO: 1) |
| | \|\|\|\|\|\|\|\|\|\|\|\|\|\|\|\| | | |
| 1 | -3′-ACCTACGTCTAGACCC-5′--- | 16 | (SEQ ID NO: 12) |

### Identity

The term “Identity” as used herein, refers to the number of nucleotides in percent of a contiguous nucleotide sequence in a nucleic acid molecule (e.g. oligonucleotide) which, at a given position, are identical to (i.e. in their ability to form Watson Crick base pairs with the complementary nucleoside) a contiguous nucleotide sequence, at a given position of a separate nucleic acid molecule (e.g. the target nucleic acid). The percentage is calculated by counting the number of aligned bases that are identical between the two sequences dividing by the total number of nucleotides in the oligonucleotide and multiplying by 100. Percent Identity=(Matches×100)/Length of aligned region. Preferably, insertions and deletions are not allowed in the calculation of % complementarity of a contiguous nucleotide sequence.

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA, 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, there are two target nucleic acids which are to be modulated by the same oligonucleotide. The target nucleic acids are i) a nucleic acid which encodes mammalian PAPD5 (target nucleic acid 1) and ii) a nucleic acid which encodes mammalian PAPD7 (target nucleic acid 2). The target nucleic acids may for example be a gene, a RNA, a mRNA, and pre-mRNA, a mature mRNA or a cDNA sequence. Suitably, the target nucleic acid encodes a PAPD5 or PAPD7 protein, in particular mammalian PAPD5 or PAPD7, such as human PAPD5 or PAPD7 (See for example table 1 and 2) which provides the pre-mRNA sequences for human, monkey, and mouse PAPD5 and PAPD7).

In some embodiments, the target nucleic acid is selected from the group consisting of SEQ ID NO: 1, 3 and/or 5 naturally occurring variants thereof (e.g. sequences encoding a mammalian PAPD5).

In some embodiments, the target nucleic acid is selected from the group consisting of SEQ ID NO: 2, 4, and/or 6 or 11 or naturally occurring variants thereof (e.g. sequences encoding a mammalian PAPD7).

TABLE 1A
Genome and assembly information for PAPD5 across species.

| | | | | Genomic coordinates | | | |
|---|---|---|---|---|---|---|---|
| Species | Chr. | Band | Strand | Start | End | ensembl_gene_id | Assembly |
| Human | 16 | q12.1 | fwd | 50152918 | 50235310 | ENSG00000121274 | GRCh38.p7 |
| Cynomolgus | 20 | | fwd | 37953893 | 38040642 | RefSeq ID: | Macaca_fascicularis_5.0 |
| monkey | | | | | | NC_022291.1 | (GCF_000364345.1) |
| mouse | 8 | C3 | fwd | 88199213 | 88259722 | ENSMUSG00000036779 | GRCm38.p5 |
| Rat | 19 | p11 | rev | 19771677 | 19832812 | ENSRNOG00000024212 | Rnor_6.0 |

TABLE 1B
Genome and assembly information for PAPD7 across species.

| | | | | Genomic coordinates | | | |
|---|---|---|---|---|---|---|---|
| Species | Chr | Band | Strand | Start | End | ensembl_gene_id | Assembly |
| Human | 5 | p15.31 | fwd | 6713007 | 6757048 | ENSG00000112941 | GRCh38.p7 |
| Cynomolgus | 6 | | fwd | 6740764 | 6790723 | RefSeq | Macaca_fascicularis_5.0 |
| monkey | | | | | | NC_022277.1 | (GCF_000364345.1) |
| mouse | 13 | B3 | rev | 69497959 | 69534617 | ENSMUSG00000034575 | GRCm38.p5 |
| Rat | 1 | p11 | fwd | 36400443 | 36433238 | ENSRNOG00000017613 | Rnor_6.0 |

Fwd = forward strand.
Rev = reverse strand.
The genome coordinates provide the pre-mRNA sequence (genomic sequence).

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the PAPD5 and PAPD7 target nucleic acid in a cell which is expressing the PAPD5 and PAPD7 target nucleic acid. The contiguous sequence of nucleobases of the oligonucleotide of the invention is typically complementary a conserved region of the PAPD5 and PAPD7 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). Further information on exemplary target nucleic acids is provided in table 2.

TABLE 2
Sequence details for PAPD5 and PAPD7 across species.

| Species | Target | RNA type | Length (nt) | SEQ ID NO |
|---|---|---|---|---|
| Human | PAPD5 | Pre-mRNA | 82393 | 1 |
| Human | PAPD7 | Pre-mRNA | 44042 | 2 |
| Cyno monkey | PAPD5 | Pre-mRNA | 86750 | 3 |
| Cyno monkey | PAPD7 | Pre-mRNA | 49960 | 4 |
| Mouse | PAPD5 | Pre-mRNA | 60510 | 5 |
| Mouse | PAPD7 | Pre-mRNA | 36659 | 6 |

### Target Sequence

The term “target sequence” as used herein refers to a sequence of nucleotides present in the target nucleic acid which comprises the nucleobase sequence which is complementary to the oligonucleotide or nucleic acid molecule of the invention. In some embodiments, the target sequence consists of a region on the target nucleic acid which is complementary to the contiguous nucleotide sequence of the oligonucleotide of the invention (i.e. a sub-sequence).

In the present invention the target sequence is present both in the human PAPD5 and human PAPD7 target nucleic acid. The target sequence may therefore be referred to as a bispecific target sequence present in both the PAPD5 and PAPD7 target nucleic acid. In advantageous embodiments the target sequence is also present in at least one additional species, such as PAPD5 and PAPD7 from cynomolgus monkey, and/or PAPD5 and PAPD7 from mouse.

The oligonucleotide or nucleic acid molecule of the invention comprises a contiguous nucleotide sequence which is complementary to or hybridizes to a region on the target nucleic acid, such as a target sequence described herein.

The target nucleic sequence to which the oligonucleotide is complementary to or hybridizes to generally comprises a stretch of contiguous nucleobases of at least 10 nucleotides. The contiguous nucleotide sequence is between 10 to 50 nucleotides, such as 12-30, such as 13 to 25, such as 14 to 20, such as 15 to 18 contiguous nucleotides.

### Naturally Occurring Variant

The term “naturally occurring variant” refers to variants of PAPD5 or PAPD7 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian PAPD5 target nucleic acid, such as a target nucleic acid selected form the group consisting of SEQ ID NO: 1, 3 or 5. In some embodiments the naturally occurring variants have at least 99% homology to the human PAPD5 target nucleic acid of SEQ ID NO: 1. In some embodiments the naturally occurring variants are the polymorphisms listed in table 3A.

In some embodiments, the naturally occurring variants have at least 95% such as at least 98% or at least 99% homology to a mammalian PAPD5 target nucleic acid, such as a target nucleic acid selected form the group consisting of SEQ ID NO: 2 or 4 or 6. In some embodiments the naturally occurring variants have at least 99% homology to the human PAPD7 target nucleic acid of SEQ ID NO: 2. In some embodiments the naturally occurring variants are the polymorphisms listed in table 3B.

Numerous single nucleotide polymorphisms are known in the PAPD5 or PAPD7 gene, for example those disclosed in Table 3A (human PAPD5 premRNA start/reference sequence is SEQ ID NO: 1) and Table 3B human PAPD7 premRNA start/reference sequence is SEQ ID NO: 2).

TABLE 3A
PAPD5 polymorphisms (naturally occurring variants)

| minor | Minor allele | |
|---|---|---|
| allele | frequency | Start on SEQ ID NO: 1 |
| G | 0.00399361 | 29 |
| G | 0.000199681 | 34 |
| T | 0.000399361 | 39 |
| A | 0.000599042 | 62 |
| A | 0.000599042 | 97 |
| G | 0.000199681 | 141 |
| A | 0.000199681 | 142 |
| T | 0.000199681 | 158 |
| A | 0.0241613 | 235 |
| A | 0.00239617 | 279 |
| — | 0.214058 | 370 |
| G | 0.000798722 | 450 |
| CAGCA | 0.000798722 | 603 |
| A | 0.0223642 | 1028 |
| C | 0.000199681 | 1044 |
| A | 0.0189696 | 1068 |
| T | 0.000199681 | 1181 |
| T | 0.0249601 | 1199 |
| T | 0.000998403 | 1268 |
| A | 0.000199681 | 1261 |
| T | 0.000599042 | 1441 |
| T | 0.000199681 | 1443 |
| C | 0.000599042 | 1469 |
| A | 0.000399361 | 1535 |

TABLE 3B
PAPD7polymorphisms (naturally occurring variants)

| | Minor allele | Start on |
|---|---|---|
| minor allele | frequency | SEQ ID NO: 2 |
| A | 0.293331 | 21 |
| T | 0.00119808 | 50 |
| T | 0.000199681 | 64 |
| A | 0.00279553 | 127 |
| A | 0.0597045 | 224 |
| G | 0.000199681 | 234 |
| T | 0.000599042 | 270 |
| A | 0.128994 | 284 |
| C | 0.000399361 | 316 |
| T | 0.000199681 | 349 |
| G | 0.00778754 | 362 |
| A | 0.000199681 | 409 |
| G | 0.000199681 | 425 |
| A | 0.000199681 | 448 |
| T | 0.000199881 | 473 |
| C | 0.000199681 | 491 |
| C | 0.327676 | 564 |
| T | 0.0203674 | 606 |
| — | 0.389577 | 837 |
| — | 0.00139778 | 1317 |
| T | 0.000599042 | 1331 |
| T | 0.000199681 | 1475 |
| T | 0.000399361 | 1483 |
| C | 0.01877 | 1673 |
| A | 0.000199681 | 1682 |
| T | 0.00339457 | 1726 |
| GGTCCTGGCCGGCGCCCGC | 0.258586 | 1736 |
| G | 0.000599042 | 1760 |
| C | 0.000199681 | 1777 |
| G | 0.000399361 | 1780 |
| T | 0.000199681 | 1852 |
| T | 0.000199681 | 1361 |
| T | 0.000199681 | 1889 |
| C | 0.000399361 | 1923 |
| G | 0.000399361 | 1962 |
| T | 0.0147764 | 1987 |
| G | 0.000998403 | 1996 |
| T | 0.000399381 | 2036 |

### Modulation of Expression

The term “modulation of expression” as used herein is to be understood as an overall term for a nucleic acid molecules ability to alter the amount of PAPD5 and PAPD7 when compared to the amount of PAPD5 and PAPD7 before administration of the nucleic acid molecule. Alternatively, modulation of expression may be determined by reference to a control experiment. It is generally understood that the control is an individual or target cell treated with a saline composition or an individual or target cell treated with a non-targeting or nucleic acid molecule (mock). It may however also be an individual treated with the standard of care.

One type of modulation is a nucleic acid molecules, such as an antisense oligonucleotides, ability to inhibit, down-regulate, reduce, remove, stop, prevent, lessen, lower, avoid or terminate expression of PAPD5 and PAPD7, e.g. by degradation of mRNA or blockage of transcription.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ sugar modified nucleosides, such as 2′ substituted nucleosides like Ome and MOE as well as 2′ to 4′ bridged nucleic acids such as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The nucleic acid molecule of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of nucleic acid molecules, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the —OH groups naturally found in RNA or DNA nucleosides. Substituents may, for example be introduced at the 2′, 3′, 4′ or 5′ positions.

### 2′Sugarmodified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

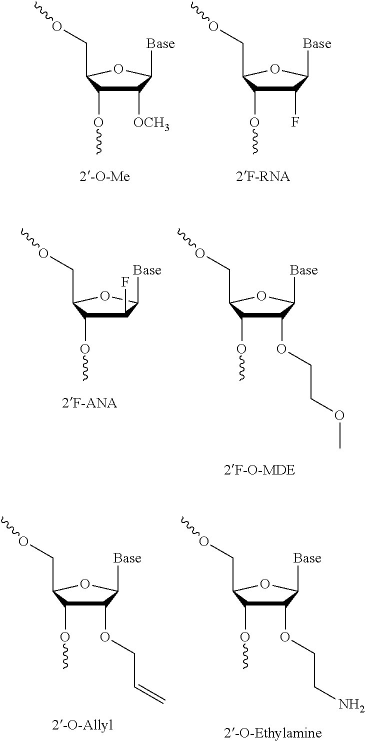

In relation to the present invention 2′ substituted does not include 2′ bridged molecules like LNA.

### Locked Nucleic Acid Nucleosides (LNA).

An “LNA nucleoside” is 2′-sugar modified nucleoside which comprises a biradical linking the C2′ and C4′ of the ribose sugar ring of a said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

In some embodiments, the 2′-sugar modified nucleoside(s) or the LNA nucleoside(s) of the oligomer of the invention has a general structure of the formula I or II:

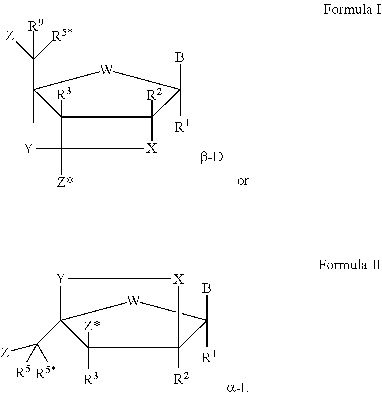

wherein W is selected from —O—, —S—, —N(R^{a})—, —C(R^{a}R^{b})—, such as, in some embodiments —O—;
B designates a nucleobase or modified nucleobase moiety;
Z designates an internucleoside linkage to an adjacent nucleoside, or a 5-terminal group;
Z* designates an internucleoside linkage to an adjacent nucleoside, or a 3′-terminal group;
X designates a group selected from the list consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, X is selected from the group consisting of: —O—, —S—, NH—, NR^{a}R^{b}, —CH₂—, CR^{a}R^{b}, —C(═CH₂)—, and —C(═CR^{a}R^{b})—

In some embodiments, X is —O—
Y designates a group selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, Y is selected from the group consisting of: —CH₂—, —C(R^{a}R^{b})—, —CH₂CH₂—, —C(R^{a}R^{b})—C(R^{a}R^{b})—, —CH₂CH₂CH₂—, —C(R^{a}R^{b})C(R^{a}R^{b})C(R^{a}R^{b})—, —C(R^{a})═C(R^{b})—, and —C(R^{a})═N—

In some embodiments, Y is selected from the group consisting of: —CH₂—, —CHR^{a}—, —CHCH₃—, CR^{a}R^{b}—
or —X—Y— together designate a bivalent linker group (also referred to as a radicle) together designate a bivalent linker group consisting of 1, 2, 3 or 4 groups/atoms selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,

In some embodiments, —X—Y— designates a biradicle selected from the groups consisting of: —X—CH₂—, —X—CR^{a}R^{b}—, —X—CHR^{a}—, —X—C(HCH₃)⁻, —O—Y—, —O—CH₂—, —S—CH₂—, —NH—CH₂—, —O—CHCH₃—, —CH₂—O—CH₂, —O—CH(CH₃CH₃)—, —O—CH₂—CH₂—, OCH₂—CH₂—CH₂—, —O—CH₂OCH₂—, —O—NCH₂—, —C(═CH₂)—CH₂—, —NR^{a}—CH₂—, N—O—CH₂, —S—CR^{a}R^{b}— and —S—CHR^{a}—.

In some embodiments —X—Y— designates —O—CH₂— or —O—CH(CH₃)—.
wherein Z is selected from —O—, —S—, and —N(R^{a})—,
and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted and where two geminal substituents R^{a} and R^{b} together may designate optionally substituted methylene (═CH₂), wherein for all chiral centers, asymmetric groups may be found in either R or S orientation,
wherein R¹, R², R³, R⁵ and R^{5*} are independently selected from the group consisting of: hydrogen, optionally substituted C₁₋₆-alkyl, optionally substituted C₂₋₆-alkenyl, optionally substituted C₂₋₆-alkynyl, hydroxy, C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted, and where two geminal substituents together may designate oxo, thioxo, imino, or optionally substituted methylene.

In some embodiments R¹, R², R³, R⁵ and R^{5*} are independently selected from C₁₋₆ alkyl, such as methyl, and hydrogen.

In some embodiments R¹, R², R³, R⁵ and R^{5*} are all hydrogen.

In some embodiments R¹, R², R³, are all hydrogen, and either R⁵ and R^{5*} is also hydrogen and the other of R⁵ and R^{5*} is other than hydrogen, such as C₁₋₆ alkyl such as methyl.

In some embodiments, R^{a} is either hydrogen or methyl. In some embodiments, when present, R^{b} is either hydrogen or methyl.

In some embodiments, one or both of R^{a} and R^{b} is hydrogen

In some embodiments, one of R^{a} and R^{b} is hydrogen and the other is other than hydrogen

In some embodiments, one of R^{a} and R^{b} is methyl and the other is hydrogen

In some embodiments, both of R^{a} and R^{b} are methyl.

In some embodiments, the biradicle —X—Y— is —O—CH₂—, W is O, and all of R¹, R², R³, R⁵ and R^{5*} are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradicle —X—Y— is —S—CH₂—, W is O, and all of R¹, R², R³, R⁵ and R^{5*} are all hydrogen. Such thio LNA nucleosides are disclosed in WO99/014226 and WO2004/046160 which are hereby incorporated by reference.

In some embodiments, the biradicle —X—Y— is —NH—CH₂—, W is O, and all of R¹, R², R³, R⁵ and R^{5*} are all hydrogen. Such amino LNA nucleosides are disclosed in WO99/014226 and WO2004/046160 which are hereby incorporated by reference.

In some embodiments, the biradicle —X—Y— is —O—CH₂—CH₂— or —O—CH₂—CH₂— CH₂—, W is O, and all of R¹, R², R³, R⁵ and R^{5*} are all hydrogen. Such LNA nucleosides are disclosed in WO00/047599 and Morita et al, Bioorganic & Med. Chem. Lett. 12 73-76, which are hereby incorporated by reference, and include what are commonly known as 2′-O-4′C-ethylene bridged nucleic acids (ENA).

In some embodiments, the biradicle —X—Y— is —O—CH₂—, W is O, and all of R¹, R², R³, and one of R⁵ and R^{5*} are hydrogen, and the other of R⁵ and R^{5*} is other than hydrogen such as C₁₋₆ alkyl, such as methyl. Such 5′ substituted LNA nucleosides are disclosed in WO2007/134181 which is hereby incorporated by reference.

In some embodiments, the biradicle —X—Y— is —O—CR^{a}R^{b}—, wherein one or both of R^{a} and R^{b} are other than hydrogen, such as methyl, W is O, and all of R¹, R², R³, and one of R⁵ and R^{5*} are hydrogen, and the other of R⁵ and R^{5*} is other than hydrogen such as C₁₋₆ alkyl, such as methyl. Such bis modified LNA nucleosides are disclosed in WO2010/077578 which is hereby incorporated by reference.

In some embodiments, the biradicle —X—Y— designate the bivalent linker group —O—CH(CH₂OCH₃)— (2′ 0-methoxyethyl bicyclic nucleic acid—Seth at al., 2010, J. Org. Chem. Vol 75(5) pp. 1569-81). In some embodiments, the biradicle —X—Y— designate the bivalent linker group —O—CH(CH₂CH₃)— (2′O-ethyl bicyclic nucleic acid—Seth at al., 2010, J. Org. Chem. Vol 75(5) pp. 1569-81). In some embodiments, the biradicle —X—Y— is —O—CHR^{a}—, W is O, and all of R¹, R², R³, R⁵ and R^{5*} are all hydrogen. Such 6′ substituted LNA nucleosides are disclosed in WO10036698 and WO07090071 which are both hereby incorporated by reference.

In some embodiments, the biradicle —X—Y— is —O—CH(CH₂OCH₃)—, W is O, and all of R¹, R², R³, R⁵ and R^{5*} are all hydrogen. Such LNA nucleosides are also known as cyclic MOEs in the art (cMOE) and are disclosed in WO07090071.

In some embodiments, the biradicle —X—Y— designate the bivalent linker group —O—CH(CH₃)—. —in either the R- or S-configuration. In some embodiments, the biradicle —X—Y— together designate the bivalent linker group —O—CH₂—O—CH₂— (Seth at al., 2010, J. Org. Chem). In some embodiments, the biradicle —X—Y— is —O—CH(CH₃)—, W is O, and all of R¹, R², R³, R⁵ and R^{5*} are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L) which are both hereby incorporated by reference).

In some embodiments, the biradicle —X—Y— is —O—CR^{a}R^{b}—, wherein in neither R^{a} or R^{b} is hydrogen, W is O, and all of R¹, R², R³, R⁵ and R^{5*} are all hydrogen. In some embodiments, R^{a} and R^{b} are both methyl. Such 6′ di-substituted LNA nucleosides are disclosed in WO 2009006478 which is hereby incorporated by reference.

In some embodiments, the biradicle —X—Y— is —S—CHR^{a}—, W is O, and all of R¹, R², R³, R⁵ and R^{5*} are all hydrogen. Such 6′ substituted thio LNA nucleosides are disclosed in WO11156202 which is hereby incorporated by reference. In some 6′ substituted thio LNA embodiments R^{a} is methyl.

In some embodiments, the biradicle —X—Y— is —C(═CH2)-C(R^{a}R^{b})—, such as —C(═CH₂)—CH₂—, or —C(═CH₂)—CH(CH₃)—W is O, and all of R¹, R², R³, R⁵ and R^{5*} are all hydrogen. Such vinyl carbo LNA nucleosides are disclosed in WO08154401 and WO09067647 which are both hereby incorporated by reference.

In some embodiments the biradicle —X—Y— is —N(—OR^{a})—, W is O, and all of R¹, R², R³, R⁵ and R^{5*} are all hydrogen. In some embodiments R^{a} is C₁₋₆ alkyl such as methyl. Such LNA nucleosides are also known as N substituted LNAs and are disclosed in WO2008/150729 which is hereby incorporated by reference. In some embodiments, the biradicle —X—Y— together designate the bivalent linker group —O—NR^{a}—CH₃— (Seth at al., 2010, J. Org. Chem). In some embodiments the biradicle —X—Y— is —N(R^{a})—, W is O, and all of R¹, R², R³, R⁵ and R^{5*} are all hydrogen. In some embodiments R^{a} is C₁₋₆ alkyl such as methyl.

In some embodiments, one or both of R⁵ and R^{5*} is hydrogen and, when substituted the other of R⁵ and R^{5*} is C₁₋₆ alkyl such as methyl. In such an embodiment, R¹, R², R³, may all be hydrogen, and the biradicle —X—Y— may be selected from —O—CH₂— or —O—C(HCR^{a})—, such as —O—C(HCH₃)—.

In some embodiments, the biradicle is —CR^{a}R^{b}—O—CR^{a}R^{b}—, such as CH₂—O—CH₂—, W is O and all of R¹, R², R³, R⁵ and R^{5*} are all hydrogen. In some embodiments R^{a} is C₁₋₆ alkyl such as methyl. Such LNA nucleosides are also known as conformationally restricted nucleotides (CRNs) and are disclosed in WO2013036868 which is hereby incorporated by reference.

In some embodiments, the biradicle is —O—CR^{a}R^{b}—O—CR^{a}R^{b}—, such as O—CH₂—O—CH₂—, W is O and all of R¹, R², R³, R⁵ and R^{5*} are all hydrogen. In some embodiments R^{a} is C₁₋₆ alkyl such as methyl. Such LNA nucleosides are also known as COC nucleotides and are disclosed in Mitsuoka et al., Nucleic Acids Research 2009 37(4), 1225-1238, which is hereby incorporated by reference.

It will be recognized than, unless specified, the LNA nucleosides may be in the beta-D or alpha-L stereoisoform.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238.

Certain examples of LNA nucleosides are presented in Scheme 1.

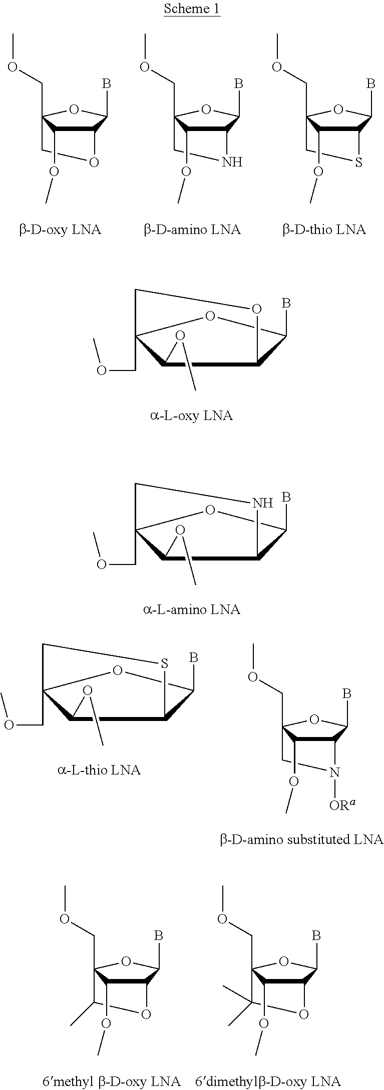

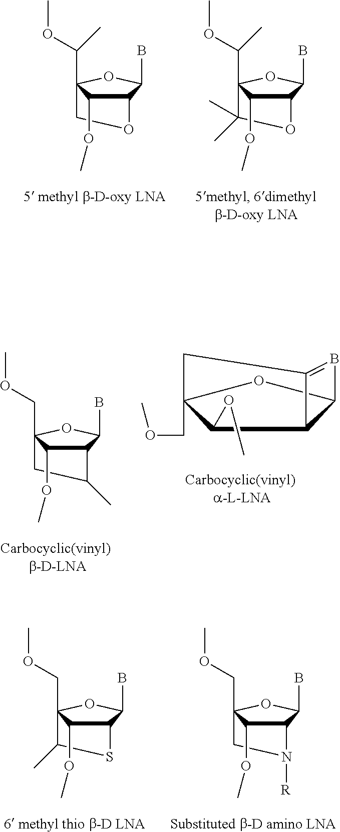

As illustrated in the examples, in some embodiments of the invention the LNA nucleosides in the oligonucleotides are beta-D-oxy-LNA nucleosides.

### Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland

### Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ′5->3′ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank. Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer—gap, Region G
Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5-methyl-cytosine (^{me}C or with an e instead of a c). Methylation of Cytosine DNA in the gap is advantageous if cg dinucleotides are present in the gap to reduce potential toxicity, the modification is not expected to have significant impact on efficacy of the oligonucleotides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

### Region G—“Gap-breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

### Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 1-6, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

Further gapmer designs are disclosed in WO2004/046160, WO2007/146511 and WO2008/113832, hereby incorporated by reference.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

In some embodiments the LNA is beta-D-oxy-LNA and the gapmer has the formula; F_{2-5 LNA, 0-2 DNA}-G_{7-11 DNA}-F′_{3-5 LNA, 0-2 DNA}

### MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

### Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and the. Flanking regions which comprise both LNA and DNA nucleoside are referred to as alternating flanks, as they comprise an alternating motif of LNA-DNA-LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak can be annotated as a series of integers, representing a number of LNA nucleosides (L) followed by a number of DNA nucleosides (D), for example

[L]₁₋₃-[D]₁₋₄-[L]₁₋₃

[L]₁₋₂-[D]₁₋₂-[L]₁₋₂-[D]₁₋₂-[L]₁₋₂

In oligonucleotide designs these will often be represented as numbers such that 2-2-1 represents 5′ [L]₂-[D]₂-[L]3′, and 1-1-1-1-1 represents 5′ [L]-[D]-[L]-[D]-[L]3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. Some examples of oligonucleotides with alternating flanks are:

[L]₁₋₅-[D]₁₋₄-[L]₁₋₃-[G]₅₋₁₆-[L]₂₋₆

[L]₁₋₂-[D]₁₋₂-[L]₁₋₂-[D]₁₋₂-[L]₁₋₂-[G]₅₋₁₆-[L]₁₋₂-[D]₁₋₃-[L]₂₋₄

[L]₁₋₅-[G]₅₋₁₆-[L]-[D]-[L]-[D]-[L]₂

with the proviso that the overall length of the gapmer is at least 12, such as at least 14 nucleotides in length.
Region D′ or D″ in an oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D‘ or D’ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitute the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

### Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs.

WO 93/07883 and WO2013/033230 provides suitable conjugate moieties, which are hereby incorporated by reference. Further suitable conjugate moieties are those capable of binding to the asialoglycoprotein receptor (ASGPR). In particular tri-valent N-acetylgalactosamine conjugate moieties are suitable for binding to the ASGPR, see for example WO 2014/076196, WO 2014/207232 and WO 2014/179620 (hereby incorporated by reference). Such conjugates serve to enhance uptake of the oligonucleotide to the liver while reducing its presence in the kidney, thereby increasing the liver/kidney ratio of a conjugated oligonucleotide compared to the unconjugated version of the same oligonucleotide.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

### Conjugate Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect one region, e.g. a conjugate moiety to another region, e.g. an oligonucleotide (e.g. the termini of region A or C).

In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable. The linker and the oligonucleotide is often attached via a phosphodiester linkage.

Biocleavable linkers (Region B) comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA.

In one embodiment the linker between the oligonucleotide and the conjugate moiety is a physiologically labile linker composed of 2 to 5 consecutive phosphodiester linked nucleosides at the 5′ or 3′ terminal of the contiguous nucleotide sequence of the antisense compound. In some embodiments the consecutive phosphodiester linkages are a dinucleotide with a sequence selected from the group consisting of AA, AT, AC, AG, TA, TT, TC, TG, CA, CT, CC, CG, GA, GT, GC, or GG. In some embodiments the consecutive phosphodiester linkages are a trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG. In specific examples phosphodiester linked CA dinucleotide, with three consecutive phosphodiester linkages, has been used as biocleavable linker between the contiguous nucleotide sequence and the conjugate moiety. Phosphodiester containing biocleavable linkers are described in more detail in WO 2014/076195 (hereby incorporated by reference). In a conjugate compound with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

Conjugates may also be linked to the oligonucleotide via non-biocleavable linkers, or in some embodiments the conjugate may comprise a non-cleavable linker which is covalently attached to the biocleavable linker. Linkers that are not necessarily biocleavable primarily serve to covalently connect a conjugate moiety to an oligonucleotide or biocleavable linker, and potentially generate some distance between the conjugate moiety and the oligonucleotide. Some example linkers (region Y) include 8-amino-3,6-dioxaoctanoic acid (ADO), succinimidyl 4-(N-maleimidomethyl)cyclohexane-I-carboxylate (SMCC), 6-aminohexanoic acid (AHEX or AHA), 6-aminohexyloxy, 4-aminobutyric acid, 4-aminocyclohexylcarboxylic acid, succinimidyl 4-(N-maleimidomethyl)cyclohexane-I-carboxy-(6-amido-caproate) (LCSMCC), succinimidyl m-maleimido-benzoylate (MBS), succinimidyl N-e-maleimido-caproylate (EMCS), succinimidyl 6-(beta-maleimido-propionamido) hexanoate (SMPH), succinimidyl N-(a-maleimido acetate) (AMAS), succinimidyl 4-(p-maleimidophenyl)butyrate (SMPB), beta-alanine (beta-ALA), phenylglycine (PHG), 4-aminocyclohexanoic acid (ACHC), beta-(cyclopropyl) alanine (beta-CYPR), amino dodecanoic acid (ADC), alylene diols, polyethylene glycols, amino acids, and the like. Non-cleavable linkers may also comprise a chain structure or an oligomer of repeating units such as ethylene glycol, amino acid units or amino alkyl groups. In some embodiments the linker (region Y) is an amino alkyl, such as a C₂-C₃₆ amino alkyl group, including, for example C₆ to C₁₂ amino alkyl groups. In some embodiments the linker (region Y) is a C₆ amino alkyl group (also termed a C6 linker). Conjugate linker groups may be routinely attached to an oligonucleotide via use of an amino modified oligonucleotide, and an activated ester group on the conjugate group. The linkage group between the amino alkyl and the oligonucleotide may for example be a phosphorothioate or a phosphodiester, or one of the other nucleoside linkage groups referred to herein. A conjugate compound of the present invention may be composed of the following regions C-B-A (Conjugate moiety-biocleavable linker-oligonucleotide/contiguous nucleotide sequence) or C-Y-B-A (conjugate moiety-non-cleavable linker-biocleavable linker-oligonucleotide/contiguous nucleotide sequence).

### Treatment

The terms “treatment”, “treating”, “treats” or the like are used herein generally mean obtaining a desired pharmacological and/or physiological effect. This effect is therapeutic in terms of partially or completely curing a disease and/or adverse effect attributed to the disease. The term “treatment” as used herein covers any treatment of a disease in a subject and includes: (a) inhibiting the disease, i.e. arresting its development like the inhibition of increase of HBsAg and/or HBeAg; or (b) ameliorating (i.e. relieving) the disease, i.e. causing regression of the disease, like the repression of HBsAg and/or HBeAg production. Thus, a compound that ameliorates and/or inhibits a HBV infection is a compound that treats a HBV invention. Preferably, the term “treatment” as used herein relates to medical intervention of an already manifested disorder, like the treatment of an already defined and manifested HBV infection.

### Prevention

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof. Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is human.

### HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Some infected persons have no symptoms during the initial infection and some develop a rapid onset of sickness with vomiting, yellowish skin, tiredness, dark urine and abdominal pain (“Hepatitis B Fact sheet N°204”. who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public-Transmission”, U.S. Centers for Disease Control and Prevention (CDC), retrieved 2011-11-29). Most of those with chronic disease have no symptoms; however, cirrhosis and liver cancer may eventually develop (Chang, 2007, Semin Fetal Neonatal Med, 12: 160-167). These complications result in the death of 15 to 25% of those with chronic disease (“Hepatitis B Fact sheet N°204”. who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

### Compound

Herein, the term “compound” means any nucleic acid molecule, such as RNAi molecules or antisense oligonucleotides according to the invention or any conjugate comprising such a nucleic acid molecule. For example, herein the compound may be a nucleic acid molecule targeting PAPD5 and PAPD7, in particular an antisense oligonucleotide.

### Composition

The term “composition” may also be used to describe a nucleic acid molecule compound. A nucleic acid molecule composition has less than 20% impurities, preferably less than 15% or 10% impurities, more preferably less than 9, 8, 7 or 6% impurities, most preferably less than 5% impurities. The impurities are typically nucleic acid molecules which are one or two nucleotides shorter (n-1 or n-2) than the primary nucleic acid molecule component.

The present invention is further described by reference to the non-limiting figures and examples.

### DETAILED DESCRIPTION OF THE INVENTION

PAPD5 and PAPD7 are non-canonical poly(A)-polymerases that belong to the superfamily of polymerase β-like nucleotidyl transferases. In PCT/EP2017/064981 PAPD5 and PAPD7 were identified as relevant targets for inhibition of an HBV infection by inhibiting the production of HBV surface antigen (HBsAg) and the expression of HBV RNA during HBV infection with two small molecules followed by confirmation with pools of siRNA compounds. In PCT/EP2017/064980 antisense oligonucleotides targeting either PAPD5 or PAPD7 were described and combined to achieve in vitro inhibition of an HBV infection.

The present invention has identified target sequences of 12 to 22 nucleotides in length which are shared between human PAPD5 and human PAPD7 mRNA in order to be able to inhibit both targets with a single nucleic acid molecule. There are around 4500 shared target sites between human PAPD5 and human PAPD7 pre-mRNA. In terms of generating a pharmaceutical acceptable molecule other parameters needs to be taken into account such as the number of off-targets as well as conservation to other species to allow in vivo proof of concept as well as meaningful pharmacokinetic/pharmacodynamic (PK/PD) modelling.

### Oligonucleotides of the Invention

The present invention has identified novel antisense oligonucleotides which are capable of inhibiting the expression of both PAPD5 and PAPD7 in vitro and in vivo. The oligonucleotides are complementary to one of three target sites of between 16 and 22 nucleotides in length which are present in both human PAPD5 and human PAPD7.

The inhibition is achieved by hybridizing the antisense oligonucleotide to a target nucleic acid encoding PAPD5 and a target nucleic acid encoding PAPD7. It is understood that the same molecule does not need to hybridize to the two targets simultaneously in order to be effective.

Target nucleic acid 1 may be a mammalian PAPD5 sequence, such as a sequence selected from the group consisting of SEQ ID NO: 1, 3 and 5.

Target nucleic acid 2 may be a mammalian PAPD7 sequence, such as a sequence selected from the group consisting of SEQ ID NO: 2, 4 and 6.

In some embodiments the antisense oligonucleotide of the invention is capable of modulating the expression of target 1 and target 2 by inhibiting or down-regulating them. Preferably, such modulation produces an inhibition of expression of at least 50% compared to the normal expression level of the targets, more preferably at least 60%, 70%, 80%, 90%, 95% or 98% inhibition compared to the normal expression level of the targets. In some embodiments oligonucleotides of the invention are capable of inhibiting expression levels of PAPD5 and PAPD7 mRNA by at least 65%-98%, such as 70% to 95%, in vitro using HeLa cells, this range of target reduction is advantageous in terms of selecting oligonucleotides with good correlation to the HBV antigen reduction, such as HBsAg and/or HBeAg reduction. In some embodiments compounds of the invention may be capable of inhibiting expression levels of PAPD5 and PAPD7 protein by at least 50% in vitro using HeLa cells. The materials and Method section and the Examples herein provide assays which may be used to measure target RNA inhibition in HeLa cells. The target modulation is triggered by the hybridization between a contiguous nucleotide sequence, such as the gapmer region, of the oligonucleotide and the target nucleic acids. In some embodiments the oligonucleotide of the invention comprises mismatches between the oligonucleotide or the contiguous nucleotide sequence and one or both of the target nucleic acids. Despite mismatches hybridization to the target nucleic acid may still be sufficient to show a desired modulation of PAPD5 and PAPD7 expression. Reduced binding affinity resulting from mismatches may advantageously be compensated by increased length of the oligonucleotide and/or an increased number of modified nucleosides capable of increasing the binding affinity to the target within the oligonucleotide sequence. Advantageously, the oligonucleotides of the present invention contain modified nucleosides capable of increasing the binding affinity, such as 2′ sugar modified nucleosides, including LNA.

An aspect of the present invention relates to an antisense oligonucleotide of 12 to 32 nucleotides in length, which comprises a contiguous nucleotide sequence of 12 to 22 nucleotides in length which is capable of inhibiting the expression of both PAPD5 and PAPD7.

In some embodiments, the oligonucleotide comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to the target nucleic acids of SEQ ID NO: 1 and SEQ ID NO: 2, or natural variants thereof.

In one embodiment the antisense oligonucleotide of the invention, or contiguous nucleotide sequence thereof is fully complementary (100% complementary) to a region of the target nucleic acids, or in some embodiments may comprise one or two mismatches between the oligonucleotide and the target nucleic acids.

In some embodiments the antisense oligonucleotide comprises a contiguous nucleotide sequence of 12 to 22 nucleotides in length with at least 93% complementary, such as fully (or 100%) complementary, to a target nucleic acid region present in SEQ ID NO: 1 and SEQ ID NO: 2.

In some embodiments the antisense oligonucleotide or the contiguous nucleotide sequence of the invention is at least 93% complementarity, such as fully (or 100%) complementary, to the target nucleic acid of SEQ ID NO: 1, SEQ ID NO: 2, SEQ ID NO: 3 and SEQ ID NO: 4.

In some embodiments the antisense oligonucleotide or the contiguous nucleotide sequence of the invention is at least 93% complementarity, such as fully (or 100%) complementary, to the target nucleic acid of SEQ ID NO: 1, SEQ ID NO: 2, SEQ ID NO: 5 and SEQ ID NO: 6.

In some embodiments the antisense oligonucleotide or the contiguous nucleotide sequence is 100% complementary to position 64669 to 69429 on SEQ ID NO: 1 and position 29514 to 29530 on SEQ ID NO: 2.

In some embodiments the antisense oligonucleotide or the contiguous nucleotide sequence is 100% complementary to position 64670 to 64685 on SEQ ID NO: 1 and position 29515 to 29530 on SEQ ID NO: 2.

In some embodiments the antisense oligonucleotide or the contiguous nucleotide sequence is 100% complementary to position 69414 to 69429 on SEQ ID NO: 1 and position 30731 to 30746 on SEQ ID NO: 2.

In some embodiments the antisense oligonucleotide or the contiguous nucleotide sequence is 100% complementary to position 759 to 781 on SEQ ID NO: 1 and position 1032 to 1054 on SEQ ID NO: 2.

In some embodiments, the antisense oligonucleotide of the invention comprises or consists of 12 to 32 nucleotides in length, such as from 14 to 25, such as 15 to 22, such as from 16 to 20 contiguous nucleotides in length.

In some embodiments, the contiguous nucleotide sequence of the antisense oligonucleotide which is complementary to the target nucleic acids comprises or consists of 12 to 22, such as from 14 to 20, such as from 16 to 20, such as from 15 to 18, such as from 16 to 18, such as from 16 to 17 contiguous nucleotides in length.

In some embodiments, the antisense oligonucleotide or the contiguous nucleotide sequence thereof comprises or consists of 22 or less nucleotides, such as 20 or less nucleotides, such as 17 or less nucleotides. It is to be understood that any range given herein includes the range endpoints. Accordingly, if an oligonucleotide is said to include from 12 to 32 nucleotides, both 12 and 32 nucleotides are included.

In some embodiments, the antisense oligonucleotide or contiguous nucleotide sequence comprises or consists of 12 to 32 nucleotides in length with at least 93% identity, preferably 100% identity, to a sequence selected from the group consisting of SEQ ID NO: 7 to 16.

In some embodiments, the antisense oligonucleotide or contiguous nucleotide sequence comprises or consists of 12 to 32 nucleotides in length with at least 93% identity, preferably 100% identity, to a sequence selected from the group consisting of SEQ ID NO: 17 to 19.

In some embodiments, the antisense oligonucleotide or contiguous nucleotide sequence comprises or consists of 12 to 32 nucleotides in length with at least 93% identity, preferably 100% identity, to a sequence of SEQ ID NO: 17 or 18.

In some embodiments, the antisense oligonucleotide or contiguous nucleotide sequence comprises or consists of 12 to 32 nucleotides in length with at least 93% identity, preferably 100% identity, to a sequence of SEQ ID NO: 19.

In a further aspect the invention relates to siRNA molecules where the antisense strand has at least 93% identity, preferably 100% identity, to a sequence selected from the group consisting of SEQ ID NO: 17 to 19.

In a further aspect the invention relates to shRNA molecules where a region of the molecule has at least 93% identity, preferably 100% identity, to a sequence selected from the group consisting of SEQ ID NO: 17 to 19.

It is understood that the contiguous nucleobase sequences (motif sequence) can be modified to for example increase nuclease resistance and/or binding affinity to the target nucleic acid.

The pattern in which the high affinity modified nucleotides are incorporated into the oligonucleotide sequence is generally termed oligonucleotide design.

The oligonucleotides of the invention are designed with modified nucleosides and DNA nucleosides. Advantageously, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA). Often used LNA LNA nucleosides are oxy-LNA, or cET.

In a further embodiment the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In an embodiment of the invention the oligonucleotide of the invention is capable of recruiting RNase H.

In the current invention an advantageous structural design is a gapmer design as described in the “Definitions” section under for example “Gapmer”, “LNA Gapmer”, “MOE gapmer” and “Mixed Wing Gapmer” “Alternating Flank Gapmer”. The gapmer design includes gapmers with uniform flanks, mixed wing flanks, alternating flanks, and gapbreaker designs. In the present invention it is advantageous if the oligonucleotide of the invention is a gapmer with an F-G-F′ design. In addition to the F-G-F′ designs described in the definitions sections one design may be where the F and F′ wing regions independently comprise 1-8 2′ sugar modified nucleosides and G is a gap region between 5 and 16 nucleosides which are capable of recruiting RNaseH.

In some embodiments the gapmer is an LNA gapmer with uniform flanks or with alternating flanks.

In some embodiments of the invention the LNA gapmer is selected from the following designs uniform flank designs 2-11-3, 2-11-4, 2-12-2, 2-12-3, 2-13-2, 2-9-6, 3-10-3, 3-10-4, 3-11-2, 3-11-3, 3-12-2, 3-9-4, 4-10-2, 4-10-3, 4-11-2, 4-7-5, 4-8-4, 4-9-3, 5-10-2, 5-6-5, 5-7-4, 5-7-5, 5-8-3, 5-8-4, 5-9-2 or 6-9-2.

In some embodiments of the invention the LNA gapmer is selected from the following alternating flanks designs 4-7-1-1-3, 4-9-1-1-2, 1-1-3-7-1-1-2, 1-1-3-9-2, 2-1-1-9-2, 2-1-1-9-3 Table 5 and 7 (Materials and Method section) lists preferred designs of each motif sequence.

In all instances the F-G-F′ design may further include region D′ and/or D″ as described in the “Definitions” section under “Region D’ or D“in an oligonucleotide”. In some embodiments the oligonucleotide of the invention has 1, 2 or 3 phosphodiester linked nucleoside units, such as DNA units, at the 5′ or 3′ end of the gapmer region. In some embodiments the oligonucleotide of the invention consists of two 5′ phosphodiester linked DNA nucleosides followed by a F-G-F′ gapmer region as defined in the “Definitions” section. In addition to the D′-F-G-F′-D″ designs described in the definitions sections one design may be an antisense oligonucleotide wherein a) the F region is between 1 and 6 nucleotides in length and consists of 2-5 identical LNA nucleosides, such as beta-D-oxy LNA or cET, and 0-3 DNA nucleosides; and b) the F′ region is between 2 and 6 nucleotides in length and consists of 2-5 identical LNA nucleosides, such as beta-D-oxy LNA or cET, and 0-3 DNA nucleosides; and c) the G region consists of between 5 and 11, such as from 7-10 DNA nucleotides and d) optionally region D′ consists of between 1 and 3 phosphodiester linked DNA nucleosides. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties, see the Conjugate section below for further details.

For certain embodiments of the invention, the oligonucleotide is selected from the group of oligonucleotide compounds with CMP ID NO: 7_1 to 783 (see oligonucleotides listed in table 5), or pharmaceutically acceptable salts thereof.

For certain embodiments of the invention, the oligonucleotide is selected from the group of oligonucleotide compounds with CMP-ID-NO: 8_1 to 881 (see oligonucleotides listed in table 5, or pharmaceutically acceptable salts thereof.

For certain embodiments of the invention, the oligonucleotide is selected from the group of oligonucleotide compounds with CMP-ID-NO: 9_1 to 912 (see oligonucleotides listed in table 5), or pharmaceutically acceptable salts thereof.

For certain embodiments of the invention, the oligonucleotide is selected from the group of oligonucleotide compounds with CMP-ID-NO: 10_1 to 10_18 (see oligonucleotides listed in table 5), or pharmaceutically acceptable salts thereof.

For certain embodiments of the invention, the oligonucleotide is selected from the group of oligonucleotide compounds with CMP-ID-NO: 11_1 to 11_26 (see oligonucleotides listed in table 5), or pharmaceutically acceptable salts thereof.

For certain embodiments of the invention, the oligonucleotide is selected from the group of oligonucleotide compounds with CMP-ID-NO: 12_1 to 12_15 (see oligonucleotides listed in table 5), or pharmaceutically acceptable salts thereof.

For certain embodiments of the invention, the oligonucleotide is selected from the group of oligonucleotide compounds with CMP-ID-NO: 13_1 or 13_2 (see oligonucleotides listed in table 5).

For certain embodiments of the invention, the oligonucleotide is selected from the group of oligonucleotide compounds with CMP-ID-NO: 14_1 to 14_13 (see oligonucleotides listed in table 5), or pharmaceutically acceptable salts thereof.

For certain embodiments of the invention, the oligonucleotide is selected from the group of oligonucleotide compounds with CMP-ID-NO: 15_1 to 15_21 (see oligonucleotides listed in table 5), or pharmaceutically acceptable salts thereof.

For certain embodiments of the invention, the oligonucleotide is selected from the group of oligonucleotide compounds with CMP-ID-NO: 16_1 to 16_5 (see oligonucleotides listed in table 5).

For certain embodiments of the invention, the oligonucleotide is selected from the group of oligonucleotide compounds with CMP-ID-NO: 17_1 to 17_183 (see oligonucleotides listed in table 7), or pharmaceutically acceptable salts thereof.

For certain embodiments of the invention, the oligonucleotide is selected from the group of oligonucleotide compounds with CMP-ID-NO: 18_1 to 18_31 or 18_250 to 18361 (see oligonucleotides listed in table 7), or pharmaceutically acceptable salts thereof.

For certain embodiments of the invention, the oligonucleotide is selected from the group of oligonucleotide compounds with CMP-ID-NO: 18_32 to 18_249 or 18_362 to 18_610 (see oligonucleotides listed in table 7), or pharmaceutically acceptable salts thereof.

For certain embodiments of the invention, the oligonucleotide is selected from the group of oligonucleotide compounds with CMP-ID-NO: 19_1 to 19_22 (see oligonucleotides listed in table 7), or pharmaceutically acceptable salts thereof.

In an embodiment of the invention the oligonucleotide is selected from the group of oligonucleotide with the compound with CMP-ID-NO: 18_1, 185, 18_10, 18_15, 18_18, 18_19, 1824, 18_27, 18_30, 18_346, 18_347, 18_357, 17_10, 17_137 and 17_139.

In an embodiment of the invention the oligonucleotide is selected from the group of oligonucleotide with the compound with CMP-ID-NO: 18_1, 18_15, 18_30, 17_10, 17_137 and 17_139.

In a further embodiment of the invention the oligonucleotide may comprise at least one stereodefined internucleoside linkages, such as a stereodefined phosphorothioate internucleoside linkage.

A key advantage of generating stereodefined oligonucleotide variants is the ability to increase the diversity across a sequence motif, and select stereodefined oligonucleotides including sub-libraries of stereodefined oligonucleotides, which have improved medicinal chemical properties as compared to a parent oligonucleotide.

In some embodiments, the improved medicinal chemical property (or improved properties) is selected from one or more of enhanced potency, enhanced specific activity, enhanced tissue uptake, enhanced cellular uptake, enhanced efficacy, altered biodistribution, reduced off-target effects, enhanced mismatch discrimination, reduced toxicity, reduced immunogenicity, altered serum protein binding, improved duration of action, and stability. Improvement in one or more property is assessed as compared to the parent oligonucleotide, such as a stereorandom parent oligonucleotide.

In some embodiments the improved property may be the ability of the oligonucleotide to modulate target expression, such as via an improved interaction with the cellular machinery involved in modulating target expression, by way of example, an enhanced RNase H activity, an improved splice modulating activity, or an improved microRNA inhibition.

In some embodiments, the improved property is RNaseH specificity, RNaseH allelic discrimination (i.e. discrimination between single nucleotide polymorphisms (SNPs) and/or RNaseH activity. In some embodiments, the improved property is other than RNaseH specificity, RNaseH allelic discrimination and/or RNaseH activity. In some embodiments the improved property is improved intracellular uptake. In some embodiments the improved property is reduced toxicity, such as cytotoxicity or hepatotoxicity.

A stereodefined oligonucleotide which exhibits one or more improved property as compared to a parent oligonucleotide, or other stereodefined oligonucleotides, is referred to as an improved phosphorothioate variant.

In an embodiment of the invention the oligonucleotide is selected from the group of oligonucleotide with the compound with CMP-ID-NO: 18_223, 18_36, 18_196, 18_188, 18_243.

In a further aspect of the invention the nucleic acid molecules, such as the antisense oligonucleotide, of the invention can be targeted directly to the liver by covalently attaching them to a conjugate moiety capable of binding to the asialoglycoprotein receptor (ASGPr), such as divalent or trivalent GalNAc cluster.

### Conjugates

Since HBV infection primarily affects the hepatocytes in the liver it is advantageous to conjugate the antisense oligonucleotides of the invention to a conjugate moiety that will increase the delivery of the oligonucleotide to the liver compared to the unconjugated oligonucleotide. In one embodiment liver targeting moieties are selected from moieties comprising cholesterol or other lipids or conjugate moieties capable of binding to the asialoglycoprotein receptor (ASGPR).

In some embodiments the invention provides a conjugate comprising an antisense oligonucleotide of the invention covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. JB. C. 1996, 271, 6686) or are readily determined using methods typical in the art.

In one embodiment the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GalNAc).

To generate the ASGPR conjugate moiety the ASPGR targeting moieties (preferably GalNAc) can be attached to a conjugate scaffold. Generally the ASPGR targeting moieties can be at the same end of the scaffold. In one embodiment the conjugate moiety consists of two to four terminal GalNAc moieties linked to a spacer which links each GalNAc moiety to a brancher molecule that can be conjugated to the antisense oligonucleotide.

In a further embodiment the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GalNAc) moieties.

The the ASPGR targeting scaffold which constitute the conjugate moiety can for example be generated by linking the GalNAc moiety to the spacer through its C—I carbon. A preferred spacer is a flexible hydrophilic spacer (U.S. Pat. No. 5,885,968; Biessen et al. J. Med. Chem. 1995 Vol. 39 p. 1538-1546). A preferred flexible hydrophilic spacer is a PEG spacer. A preferred PEG spacer is a PEG3 spacer. The branch point can be any small molecule which permits attachment of two to three GalNAc moieties or other asialoglycoprotein receptor targeting moieties and further permits attachment of the branch point to the oligonucleotide, such constructs are termed GalNAc clusters or GalNAc conjugate moieties. An exemplary branch point group is a di-lysine. A di-lysine molecule contains three amine groups through which three GalNAc moieties or other asialoglycoprotein receptor targeting moieties may be attached and a carboxyl reactive group through which the di-lysine may be attached to the oligomer. Khorev, et al 2008 Bioorg. Med. Chem. Vol 16, pp. 5216 also describes the synthesis of a suitable trivalent brancher. Other commercially available branchers are 1,3-bis-[5-(4,4′-dimethoxytrityloxy)pentylamido]propyl-2-[(2-cyanoethyl)-(N,N-diisopropyl)]phosphoramidite (Glen Research Catalogue Number: 10-1920-xx); tris-2,2,2-[3-(4,4′-dimethoxytrityloxy)propyloxymethyl]ethyl-[(2-cyanoethyl)-(N,N-diisopropyl)]-phosphoramidite (Glen Research Catalogue Number: 10-1922-xx); and tris-2,2,2-[3-(4,4′-dimethoxytrityloxy)propyloxymethyl]methyleneoxypropyl-[(2-cyanoethyl)-(N, N-diisopropyl)]-phosphoramidite; and 1-[5-(4,4′-dimethoxy-trityloxy)pentylamido]-3-[5-fluorenomethoxy-carbonyl-oxy-pentylamido]-propyl-2-[(2-cyanoethyl)-(N,N-diisopropyl)]-phosphoramidite (Glen Research Catalogue Number: 10-1925-xx).

Other GalNAc conjugate moieties can include, for example, those described in WO 2014/179620 and WO 2016/055601 and PCT/EP2017/059080 (hereby incorporated by reference), as well as small peptides with GalNAc moieties attached such as Tyr-Glu-Glu-(aminohexyl GalNAc)3 (YEE(ahGalNAc)3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GalNAc conjugate moiety, may be attached to the 3′- or 5′-end of the oligonucleotide using methods known in the art. In one embodiment the ASGPR conjugate moiety is linked to the 5′-end of the oligonucleotide.

One or more linkers may be inserted between the conjugate moiety (such as at the brancher molecule) and the oligonucleotide. It is advantageous to have a biocleavable linker between the conjugate moiety and the antisense oligonucleotide, optionally in combination with a non-cleavable linker such as a C6 linker. The linker(s) may be selected from the linkers described in the “Definitions” section under “Conjugate linkers” in particular biocleavable region D′ or D″ linkers are advantageous.

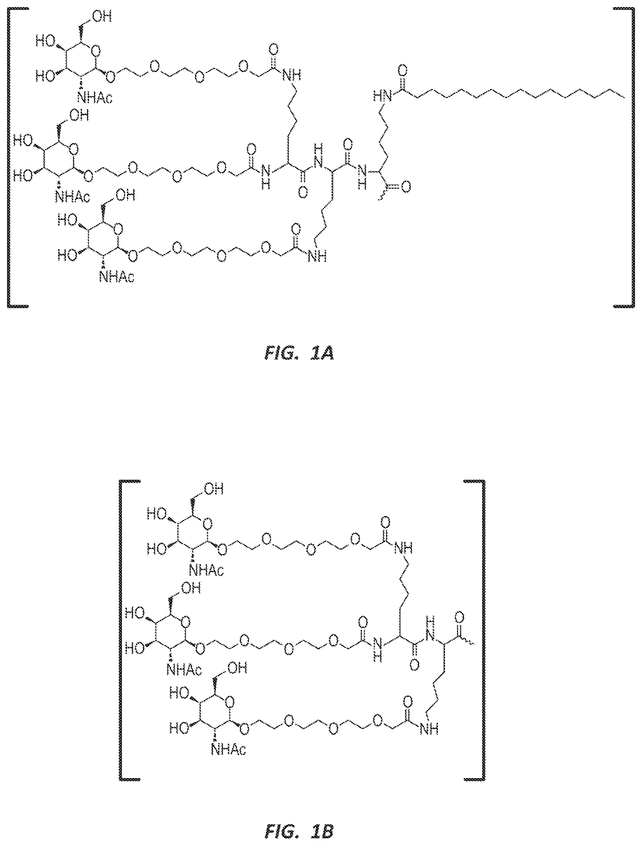

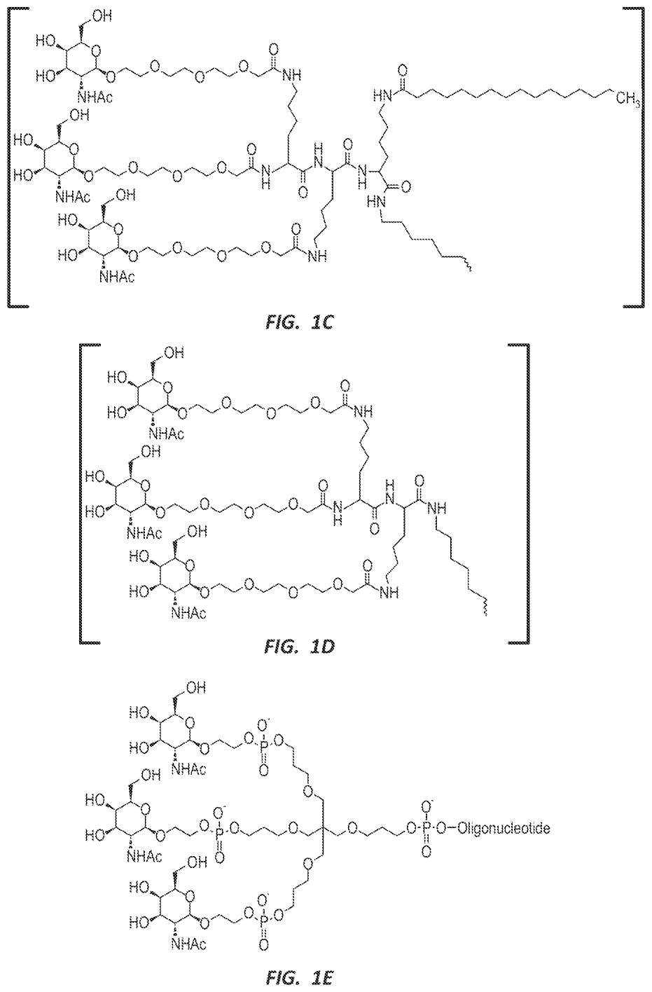

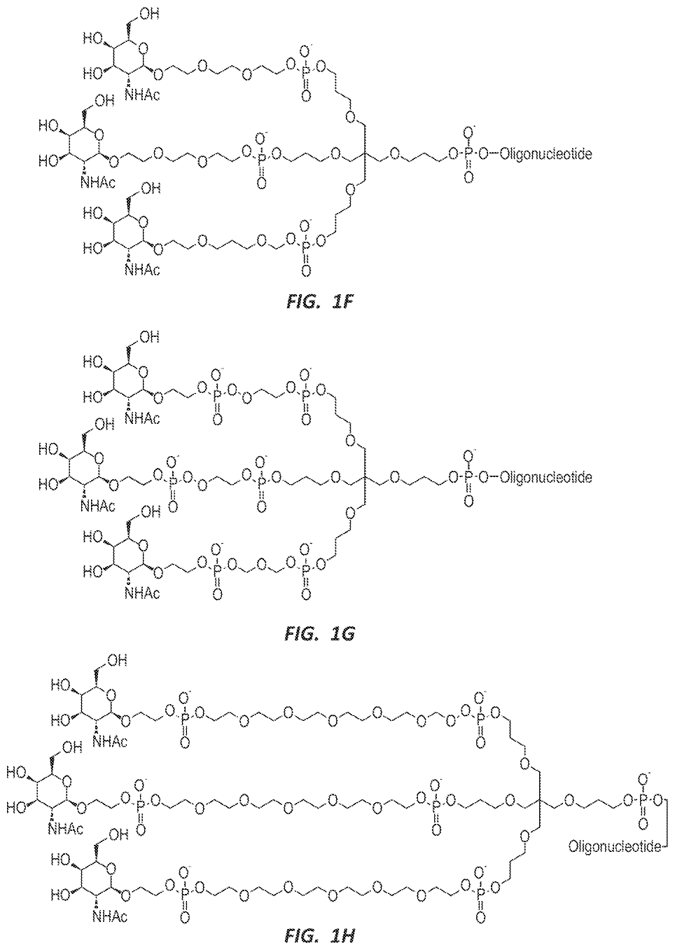

In one embodiment the conjugate moiety is a tri-valent N-acetylgalactosamine (GalNAc), such as those shown in FIG. 1, in particular as shown in FIG. 1D.

In an embodiment of the invention the conjugate compound is selected from the group of compounds in table 9 in the Material and Method section.

In an embodiment of the invention the conjugate compound is CMP-ID-NO: 20_12.

In an embodiment of the invention the conjugate compound is CMP-ID-NO 20_13.

In an embodiment of the invention the conjugate compound is CMP-ID-NO 20_14.

In an embodiment of the invention the conjugate compound is CMP-ID-NO 20_15.

In an embodiment of the invention the conjugate compound is CMP-ID-NO 20_16.

In an embodiment of the invention the conjugate compound is CMP-ID-NO 20_18.

In an embodiment of the invention the conjugate compound is CMP-ID-NO 20_20.

In an embodiment of the invention the conjugate compound is CMP-ID-NO 20_21.

In an embodiment of the invention the conjugate compound is CMP-ID-NO 20_22.

In an embodiment of the invention the conjugate compound is CMP-ID-NO 20_30.

In an embodiment of the invention the conjugate compound is CMP-ID-NO 20_35.

In an embodiment of the invention the conjugate compound is CMP-ID-NO 20_36.

In an embodiment of the invention the conjugate compound is CMP-ID-NO 21_2.

In an embodiment of the invention the conjugate compound is CMP-ID-NO 21_33.

In an embodiment of the invention the conjugate compound is CMP-ID-NO 21_34.

### Method of Manufacture

In a further aspect, the invention provides methods for manufacturing the antisense oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Compositions

In a further aspect, the invention provides pharmaceutical compositions comprising an antisense oligonucleotides and/or conjugate compounds of the invention or salts thereof and a pharmaceutically acceptable diluent, carrier, salt and/or adjuvant. A typical pharmaceutical composition is prepared by mixing antisense oligonucleotide or conjugate compound of the invention and a diluent, carrier, or excipient.

A pharmaceutically acceptable diluent includes phosphate-buffered saline (PBS). In some embodiments the pharmaceutically acceptable diluent is sterile phosphate buffered saline. In some embodiments the oligonucleotide is used in the pharmaceutically acceptable diluent at a concentration of 50-300 μM solution.

For nucleic acid molecules, antisense oligonucleotides and conjugate compound comprising these suitable formulations are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt or potassium salt.

### Applications

The oligonucleotides of the invention may be utilized as research reagents for, for example, diagnostics, therapeutics and prophylaxis.

In research, such oligonucleotides may be used to specifically modulate the synthesis of PAPD5 and PAPD7 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

Also encompassed by the present invention is an in vivo or in vitro method for modulating PAPD5 and PAPD7 expression in a target cell which is expressing PAPD5 and PAPD7, said method comprising administering an antisense oligonucleotide, conjugate compound or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is present in in the liver. The target cell may be a hepatocyte.

One aspect of the present invention is related the antisense oligonucleotides, conjugate compounds or pharmaceutical compositions of the invention for use as a medicament.

In an aspect of the invention the antisense oligonucleotide, conjugate compound or pharmaceutical composition of the invention is capable of inhibiting the propagation of HBV. In particular the antisense oligonucleotide is capable of affecting one or more of the following parameters i) reduce the expression of viral RNA; ii) reduce the production of viral DNA (HBV DNA) derived from viral RNA (HBV RNA), iii) reduce the production of new viral particles (HBV particles); iv) reduce production of HBV antigens, in particular HBsAg and/or HBeAg.

For example, an antisense oligonucleotide that inhibits propagation of HBV may reduce i) the expression of viral RNA (HBV RNA) by at least 40% such as 50%, 60%, 70%, 80%, or 90% reduction compared to controls; ii) the production of viral DNA (HBV DNA) by at least 40% such as 50%, 60%, 70%, 80%, or 90% reduction compared to controls; iii) the production of new viral particles (HBV particles) by at least 40% such as 50%, 60%, 70%, 80%, or 90% reduction compared to controls; or iv) the production and/or secretion of HBsAg and/or HBeAg by at least 50%, such as at least 60%, 70%, 80%, 90% or even up to complete depletion of one or both of the antigens compared to controls. The controls may be untreated cells or animals or cell or animal treated with an appropriate control.

Inhibition of propagation of HBV may be measured in vitro using HBV infected dHepaRG cells or ASGPR-dHepaRG cells or in vivo for oligonucleotides complementary to mouse PAPD5 and PAPD7 using the AAV/HBV mouse model as described in the Materials and Methods section. Inhibition of secretion of HBsAg and/or HBeAg may be measured by ELISA, e.g. by using the CLIA ELISA Kit (Autobio Diagnostic) according to the manufacturers' instructions. Inhibition of production of intracellular HBV mRNA may be measured by real-time PCR, e.g. as described in the Materials and Methods section. Further methods for evaluating whether a test compound inhibits propagation of HBV are measuring secretion of HBV DNA by RT-qPCR e.g. as described in WO 2015/173208 or as described in Materials and method section; Northern Blot; in-situ hybridization, or immuno-fluorescence.

Due to the reduction of HBsAg secretion the antisense oligonucleotides, conjugate compounds or pharmaceutical compositions of the present invention can be used to inhibit development of or in the treatment of HBV infection. In particular, due to inhibition of HBeAg secretion, the antisense oligonucleotides, conjugate compounds or pharmaceutical compositions of the present invention more efficiently inhibits development of or treats a chronic HBV infection as compared to a compound that only reduces secretion of HBsAg. In addition, reducing HBeAg in an expecting mother may also inhibit the development of a chronic HBV infection of her child. Thus, due to the reduction of HBeAg secretion the antisense oligonucleotides, conjugate compounds or pharmaceutical compositions of the present invention inhibits development of a chronic HBV infection (such as development of a chronic HBV infection in the offspring of an HBV infected mother) and reduces the infectiousness of a HBV infected person.

Accordingly, one aspect of the present invention is related to use of the antisense oligonucleotides, conjugate compounds or pharmaceutical compositions of the invention to reduce secretion of HBsAg and HBeAg in an HBV infected individual. It is advantageous if the antisense oligonucleotides, conjugate compounds or pharmaceutical compositions of the invention are capable of reducing HBsAg expression from HBV DNA integrated into the host genome.

A further aspect of the invention relates to the use of the antisense oligonucleotides, conjugate compounds or pharmaceutical compositions of the invention to inhibit development of or treat a chronic HBV infection.

A further aspect of the invention relates to the use of the antisense oligonucleotides, conjugate compounds or pharmaceutical compositions of the invention to and reduces the infectiousness of a HBV infected person. In a particular aspect of the invention, the antisense oligonucleotides, conjugate compounds or pharmaceutical compositions of the invention inhibits development of a chronic HBV infection in the offspring of a HBV infected mother. This mother is preferably HBeAg positive.

The subject to be treated with the antisense oligonucleotides, conjugate compounds or pharmaceutical compositions of the invention (or which prophylactically receives antisense oligonucleotides, conjugate compounds or pharmaceutical compositions of the present invention) is preferably a human, more preferably a human patient who is HBsAg positive and/or HBeAg positive, even more preferably a human patient that is HBsAg positive and HBeAg positive. Said human patient may be an expected mother, e.g. an expected mother who is HBeAg positive and/or HBsAg positive, more preferably an expected mother who is HBeAg positive and HBsAg positive.

Accordingly, the present invention relates to a method of treating and/or preventing a HBV infection, wherein the method comprises administering an effective amount of the antisense oligonucleotides, conjugate compounds or pharmaceutical compositions of the invention.

The invention also provides for the use of a nucleic acid molecule, an antisense oligonucleotide, a conjugate compound or a pharmaceutical composition of the invention for the manufacture of a medicament, in particular a medicament for use in the treatment or prevention of HBV infection or chronic HBV infection or reduction of the infectiousness of a HBV infected person. In preferred embodiments the medicament is manufactured in a dosage form for subcutaneous administration.

The invention also provides for the use of a nucleic acid molecule, an antisense oligonucleotide, a conjugate compound, the pharmaceutical composition of the invention for the manufacture of a medicament wherein the medicament is in a dosage form for intravenous administration.

The nucleic acid molecule, antisense oligonucleotide or the pharmaceutical composition of the invention may be used in a combination therapy. For example, nucleic acid molecule, antisense oligonucleotide, or the pharmaceutical composition of the invention may be combined with other anti-HBV agents such as interferon alpha-2b, interferon alpha-2a, and interferon alphacon-1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697 and WO 2014/179629), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Administration

The antisense oligonucleotides, conjugate compounds or pharmaceutical composition of the invention is formulated, dosed, and administered in a fashion consistent with good medical practice. Factors for consideration in this context include the particular mammal being treated, the clinical condition of the individual patient, the site of delivery of the agent, the method of administration, the scheduling of administration, the age and sex of the patients and other factors known to medical practitioners. Herein, an “effective amount” (also known as “(therapeutically) effective dose”) means the amount of a compound that will elicit the biological or medical response of a subject that is being sought by a medical doctor or other clinician. The “effective amount” of an antisense oligonucleotide, conjugate compound or pharmaceutical composition of the invention, will be governed by such considerations, and is the minimum amount necessary to inhibit HBsAg and/or HBeAg. For example, such amount may be below the amount that is toxic to the cells of the recipient, or to the mammal as a whole.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The nucleic acid molecules or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In a preferred embodiment the nucleic acid molecule, antisense oligonucleotide, conjugate compounds or pharmaceutical compositions of the present invention are administered by a parenteral route including intravenous, intraarterial, subcutaneous, intraperitoneal or intramuscular injection or infusion. In one embodiment the active oligonucleotide or oligonucleotide conjugate is administered intravenously. With GalNAc conjugated compounds it may be advantageous to administer subcutaneously in order to delay saturation of the ASGP reseptor.

### Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

By way of example, the oligomer or the oligomer conjugate of the present invention may be used in combination with other actives, such as oligonucleotide-based antivirals—such as sequence specific oligonucleotide-based antivirals—acting either through antisense (including other LNA oligomers), siRNAs (such as ARC520), aptamers, morpholinos or any other antiviral, nucleotide sequence-dependent mode of action.

By way of further example, the oligomer or the oligomer conjugate of the present invention may be used in combination with other actives, such as immune stimulatory antiviral compounds, such as interferon (e.g. pegylated interferon alpha), TLR7 agonists (e.g. GS-9620), or therapeutic vaccines.

By way of further example, the oligomer or the oligomer conjugate of the present invention may be used in combination with other actives, such as small molecules, with antiviral activity. These other actives could be, for example, nucleoside/nucleotide inhibitors (eg entecavir or tenofovir disoproxil fumarate), encapsidation inhibitors, entry inhibitors (eg Myrcludex B).

In certain embodiments, the additional therapeutic agent may be an HBV agent, an Hepatitis C virus (HCV) agent, a chemotherapeutic agent, an antibiotic, an analgesic, a nonsteroidal anti-inflammatory (NSAID) agent, an antifungal agent, an antiparasitic agent, an anti-nausea agent, an anti-diarrheal agent, or an immunosuppressant agent.

In particular related embodiments, the additional HBV agent may be interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In other particular related embodiments, the additional HCV agent may be interferon alpha-2b, interferon alpha-2a, and interferon alphacon-1 (pegylated and unpegylated); ribavirin; pegasys; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

### Embodiments of the Invention

The following embodiments of the present invention may be used in combination with any other embodiments described herein.

1. A nucleic acid molecule of 12 to 32 nucleotides in length, which comprises a contiguous nucleotide sequence of 12 to 22 nucleotides in length which is capable of inhibiting the expression of both PAPD5 and PAPD7.

2. The nucleic acid molecule of embodiment 1, wherein the contiguous nucleotide sequence is at least 93% complementarity to target nucleic acid of SEQ ID NO: 1 and SEQ ID NO: 2.

3. The nucleic acid molecule of embodiment 1 or 2, wherein the contiguous nucleotide sequence is at least 100% complementarity to target nucleic acid of SEQ ID NO: 1 and SEQ ID NO: 2.

4. The nucleic acid molecule of embodiment 1 or 3, wherein the contiguous nucleotide sequence is complementary to target nucleic acid of SEQ ID NO: 1, SEQ ID NO: 2, SEQ ID NO: 3 and SEQ ID NO: 4.

5. The nucleic acid molecule of embodiment 1 or 3, wherein the contiguous nucleotide sequence is complementary to target nucleic acid of SEQ ID NO: 1, SEQ ID NO: 2, SEQ ID NO: 5 and SEQ ID NO: 6.

6. The nucleic acid molecule of embodiment 1 to 3 or 5, wherein the nucleic acid molecule is complementary to position 759 to 781 on SEQ ID NO: 1 and position 1032 to 1054 on SEQ ID NO: 2.

7. The nucleic acid molecule of embodiment 1 to 4, wherein the nucleic acid molecule is complementary to position 64669 to 69429 on SEQ ID NO: 1 and position 29514 to 29530 on SEQ ID NO: 2.

8. The nucleic acid molecule of embodiment 1 to 4, wherein the nucleic acid molecule is complementary to position 69414 to 69429 on SEQ ID NO: 1 and position 30731 to 30746 on SEQ ID NO: 2.

9. The nucleic acid molecule of embodiment 1 to 8 is capable of hybridizing to a target nucleic acid of SEQ ID NO: 1 and SEQ ID NO: 2 with a ΔG° below −15 kcal.

10. The nucleic acid molecule of embodiment 2 to 9, wherein the target nucleic acid is RNA.

11. The nucleic acid molecule of embodiment 10, wherein the RNA is pre-mRNA.

12. The nucleic acid molecule of embodiment 1-11, wherein the nucleic acid molecule is selected from antisense oligonucleotide, siRNA or shRNA.

13. The nucleic acid molecule of embodiment 1-11, wherein the nucleic acid molecule is a single stranded antisense oligonucleotide.

14. The antisense oligonucleotide of embodiment 12 or 13, wherein the contiguous nucleotide sequence comprises or consists of at least 14 contiguous nucleotides, particularly 15, 16, 17, 18, 19 or 20 contiguous nucleotides.

15. The antisense oligonucleotide of embodiment 12 or 13, wherein the contiguous nucleotide sequence comprises or consists of from 14 to 20 nucleotides.

16. The antisense oligonucleotide of embodiment 15, wherein the contiguous nucleotide sequence comprises or consists of from 16 to 18 nucleotides.

17. The antisense oligonucleotide of embodiment 1 to 16, wherein the oligonucleotide comprises or consists of 14 to 25 nucleotides in length.

18. The antisense oligonucleotide of embodiment 17, wherein the antisense oligonucleotide comprises or consists of 15 to 22 nucleotides in length.

19. The antisense oligonucleotide of embodiment 17 or 18, wherein the antisense oligonucleotide comprises or consists of 16 to 20 nucleotides in length.

20. The antisense oligonucleotide of embodiment 12-19, wherein the contiguous nucleotide sequence comprises or consists of a sequence selected from the group consisting of SEQ ID NO: 7, 8, 9, 10, 11, 12, 13, 14, 15, 16, 17, 18 and 19.

21. The antisense oligonucleotide of embodiment 12-20, wherein the contiguous nucleotide sequence comprises or consists of a sequence selected from the group consisting of SEQ ID NO: 7, 8, 9, 10, 11, 12, 13, 14, 15 and 16.

22. The antisense oligonucleotide of embodiment 12-20, wherein the contiguous nucleotide sequence comprises or consists of a sequence selected from SEQ ID NO: 17 or SEQ ID NO: 18.

23. The antisense oligonucleotide of embodiment 12-20, wherein the contiguous nucleotide sequence comprises or consists of SEQ ID NO: 19.

24. The antisense oligonucleotide of embodiment 12-23, wherein the contiguous nucleotide sequence has zero to three mismatches compared to the target nucleic acids it is complementary to.

25. The antisense oligonucleotide of embodiment 24, wherein the contiguous nucleotide sequence has one mismatch compared to the target nucleic acids.

26. The antisense oligonucleotide of embodiment 24, wherein the contiguous nucleotide sequence has two mismatches compared to the target nucleic acids.

27. The antisense oligonucleotide of embodiment 24, wherein the contiguous nucleotide sequence is fully complementary to both target nucleic acid sequences.

28. The antisense oligonucleotide of embodiment 12-27, comprising one or more modified nucleosides.

29. The antisense oligonucleotide of embodiment 28, wherein the one or more modified nucleoside is a high-affinity modified nucleosides.

30. The antisense oligonucleotide of embodiment 28 or 29, wherein the one or more modified nucleoside is a 2′ sugar modified nucleoside.

31. The antisense oligonucleotide of embodiment 30, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.

32. The antisense oligonucleotide of embodiment 28-31, wherein the one or more modified nucleoside is a LNA nucleoside.

33. The antisense oligonucleotide of embodiment 32, wherein the modified LNA nucleoside is selected from oxy-LNA, amino-LNA, thio-LNA, cET, and ENA.

34. The antisense oligonucleotide of embodiment 32 or 33, wherein the modified LNA nucleoside is oxy-LNA with the following 2′-4′ bridge —O—CH₂—.

35. The antisense oligonucleotide of embodiment 34, wherein the oxy-LNA is beta-D-oxy-LNA.

36. The antisense oligonucleotide of embodiment 32 or 33, wherein the modified LNA nucleoside is cET with the following 2′-4′ bridge —O—CH(CH₃)—.

37. The antisense oligonucleotide of embodiment 36, wherein the cET is (S)cET, i.e. 6′(S)methyl-beta-D-oxy-LNA.

38. The antisense oligonucleotide of embodiment 32 or 33, wherein the LNA is ENA, with the following 2′-4′ bridge —O—CH₂—CH₂—.

39. The antisense oligonucleotide of any one of embodiments 12-33, wherein the antisense oligonucleotide comprises at least one modified internucleoside linkage.

40. The antisense oligonucleotide of embodiment 39, wherein the modified internucleoside linkage is nuclease resistant.

41. The antisense oligonucleotide of embodiment 39 or 40, wherein at least 75% of the internucleoside linkages within the contiguous nucleotide sequence are phosphorothioate internucleoside linkages or boranophosphate internucleoside linkages.

42. The antisense oligonucleotide of embodiment 39 or 40, wherein all the internucleoside linkages within the contiguous nucleotide sequence are phosphorothioate internucleoside linkages.

43. The antisense oligonucleotide of embodiment 41 or 42, wherein at least one of the phosphorothioate internucleoside linkages are stereodefined

44. The antisense oligonucleotide of embodiment 12-43, wherein the antisense oligonucleotide is capable of recruiting RNase H.

45. The antisense oligonucleotide of embodiment 44, wherein the antisense oligonucleotide or the contiguous nucleotide sequence is a gapmer.

46. The antisense oligonucleotide of embodiment 45, wherein the gapmer has the formula 5′-F-G-F′-3′, where the F and F′ wing regions independently comprise or consist of 1-7 2′ sugar modified nucleosides in accordance with embodiments 31 to 38 and G is a region between 5 and 16 nucleosides which are capable of recruiting RNaseH.

47. The antisense oligonucleotide of embodiment 46, wherein each wing (F and F′) is characterized by having at least one 2′ sugar modified nucleoside at the 5′ terminal and the 3′ terminal of the wing and the G region has at least one DNA nucleoside adjacent to the wing regions (e.g. 5′ and 3′ terminal of the G region).

48. The antisense oligonucleotide of embodiment 46 or 47, wherein all the 2′ sugar modified nucleosides in region F and F′ are identical LNA nucleosides.

49. The oligonucleotide of embodiment 46-48, wherein
a. the F region is between 1 and 6 nucleotides in length and consists of 1-5 identical LNA nucleosides and 0-3 DNA nucleosides; and
b. the F′ region is between 2 and 6 nucleotides in length and consists of 2-5 identical LNA nucleosides and 0-3 DNA nucleosides; and
c. the G region is between 5 and 11 nucleotides which are capable of recruiting RNaseH, and
d. optionally a D′ region with 1 to 3 phosphodiester linked DNA nucleosides are positioned at the 5′ end of the F region

50. The antisense oligonucleotide of embodiment 47, wherein region F and F′ consist of identical LNA nucleosides.

51. The antisense oligonucleotide of embodiment 46-48, wherein all the 2′ sugar modified nucleosides in region F and F′ are oxy-LNA nucleosides.

52. The antisense oligonucleotide of embodiment 46 or 47, wherein at least one of region F or F′ further comprises at least one 2′ substituted modified nucleoside independently selected from the group consisting of 2′-O-alkyl-RNA, 2′-O-methyl-RNA, 2′-alkoxy-RNA, 2′-O-methoxyethyl-RNA, 2′-amino-DNA and 2′-fluoro-DNA.

53. The antisense oligonucleotide of embodiment 46-52, wherein the RNaseH recruiting nucleosides in region G are independently selected from DNA, alpha-L-LNA, C4′ alkylated DNA ANA and 2′ F-ANA and UNA.

54. The antisense oligonucleotide of embodiment 53, wherein the nucleosides in region G is DNA and/or alpha-L-LNA nucleosides.

55. The antisense oligonucleotide of embodiment 46 or 53 or 54, wherein region G consists of at least 75% DNA nucleosides.

56. The antisense oligonucleotide of embodiment 55, where all the nucleosides in region G are DNA nucleosides.

57. The antisense oligonucleotide of embodiment 12-55, wherein the antisense oligonucleotide is selected from CMP ID NO: 7_1 to 7_83, or pharmaceutically acceptable salts thereof.

58. The antisense oligonucleotide of embodiment 12-55, wherein the antisense oligonucleotide is selected from CMP ID NO: 8_1 to 8_81, or pharmaceutically acceptable salts thereof.

59. The antisense oligonucleotide of embodiment 12-55, wherein the antisense oligonucleotide is selected from CMP ID NO: 9_1 to 9_12, or pharmaceutically acceptable salts thereof.

60. The antisense oligonucleotide of embodiment 12-55, wherein the antisense oligonucleotide is selected from CMP ID NO: 10_1 to 10_18, or pharmaceutically acceptable salts thereof.

61. The antisense oligonucleotide of embodiment 12-55, wherein the antisense oligonucleotide is selected from CMP ID NO: 11_1 to 11_26, or pharmaceutically acceptable salts thereof.

62. The antisense oligonucleotide of embodiment 12-55, wherein the antisense oligonucleotide is selected from CMP ID NO: 12_1 to 12_15, or pharmaceutically acceptable salts thereof.

63. The antisense oligonucleotide of embodiment 12-55, wherein the antisense oligonucleotide is selected from CMP ID NO: 13_1 or 132, or pharmaceutically acceptable salts thereof.

64. The antisense oligonucleotide of embodiment 12-55, wherein the antisense oligonucleotide is selected from CMP ID NO: 14_1 to 14_13, or pharmaceutically acceptable salts thereof.

65. The antisense oligonucleotide of embodiment 12-55, wherein the antisense oligonucleotide is selected from CMP ID NO: 15_1 to 15_21, or pharmaceutically acceptable salts thereof.

66. The antisense oligonucleotide of embodiment 12-55, wherein the antisense oligonucleotide is selected from CMP ID NO: 16_1 to 16_5, or pharmaceutically acceptable salts thereof.

67. The antisense oligonucleotide of embodiment 12-55, wherein the antisense oligonucleotide is selected from CMP ID NO: 17_1 to 17_183, or pharmaceutically acceptable salts thereof.

68. The antisense oligonucleotide of embodiment 12-55, wherein the antisense oligonucleotide is selected from CMP ID NO: 18_1 to 18_31 or 18_250 to 18_361, or pharmaceutically acceptable salts thereof.

69. The antisense oligonucleotide of embodiment 68, wherein the antisense oligonucleotide is selected from CMP ID NO: 18_1, 185, 18_10, 18_15, 18_18, 18_19, 1824, 1827, 1830, 18346, 18_347, 18_357, 17_10, 17_137 and 17_139, or pharmaceutically acceptable salts thereof.

70. The antisense oligonucleotide of embodiment 69, wherein the antisense oligonucleotide is selected from CMP ID NO: 18_1, 18_15, 1827, 1830, 17_10, 17_137 and 17_139.

71. The antisense oligonucleotide of embodiment 12-55, wherein the antisense oligonucleotide is selected from CMP ID NO: 18_32 to 18_249 or 18_362 to 18_610, or pharmaceutically acceptable salts thereof.

72. The antisense oligonucleotide of embodiment 71, wherein the antisense oligonucleotide is selected from CMP ID NO: 18_223, 1836, 18_196, 18_188 and 18_243.

73. The antisense oligonucleotide of embodiment 12-55, wherein the antisense oligonucleotide is selected from CMP ID NO: 19_1 to 19_22, or pharmaceutically acceptable salts thereof.

74. A conjugate compound comprising a nucleic acid molecule according to any one of claims 1 to 11 or an antisense oligonucleotide according to any one of claims 12-57, and at least one conjugate moiety covalently attached to said antisense oligonucleotide.

75. The conjugate compound of embodiment 74, wherein the conjugate moiety is selected from carbohydrates, cell surface receptor ligands, drug substances, hormones, lipophilic substances, polymers, proteins, peptides, toxins, vitamins, viral proteins or combinations thereof.

76. The conjugate compound of embodiment 74 or 75, wherein the conjugate moiety is capable of binding to the asialoglycoprotein receptor.

77. The conjugate compound of embodiment 76, wherein the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine.

78. The conjugate compound of embodiment 77, wherein the asialoglycoprotein receptor targeting moiety is N-acetylgalactosamine (GalNAc).

79. The conjugate compound of embodiment 77 or 78, wherein the conjugate moiety is mono-valent, di-valent, tri-valent or tetra-valent with respect to asialoglycoprotein receptor targeting moieties.

80. The conjugate compound of embodiment 79, wherein the conjugate moiety consists of two to four terminal GalNAc moieties and a spacer linking each GalNAc moiety to a brancher molecule that can be conjugated to the antisense compound.

81. The conjugate compound of embodiment 80, wherein the spacer is a PEG spacer.

82. The conjugate compound of embodiment 76 to 81, wherein the conjugate moiety is a tri-valent N-acetylgalactosamine (GalNAc) moiety.

83. The conjugate compound of embodiment 76 to 82, wherein the conjugate moiety is selected from one of the trivalent GalNAc moieties in FIG. 1.

84. The conjugate compound of embodiment 83, wherein the conjugate moiety is the trivalent GalNAc moiety in FIG. 1D.

85. The conjugate compound of embodiment 74-84, comprising a linker which is positioned between the nucleic acid molecule or the antisense oligonucleotide and the conjugate moiety.

86. The conjugate compound of embodiment 85, wherein the linker is a physiologically labile linker.

87. The conjugate compound of embodiment 86, wherein the physiologically labile linker is nuclease susceptible linker.

88. The oligonucleotide conjugate of embodiment 86 or 87, wherein the physiologically labile linker is composed of 2 to 5 consecutive phosphodiester linkages.

89. The conjugate compound of embodiment 86 to 88, wherein the antisense oligonucleotide has the formula D′-F-G-F′ or F-G-F′-D″, wherein F, F′ and G are as defined in embodiments 46-56 and D′ or D″ comprises 1, 2 or 3 DNA nucleosides with phosphodiester internucleoside linkages.

90. The oligonucleotide conjugate of embodiment 88 or 89, wherein at least two consecutive phosphodiester internucleoside linkages are associated with a CA dinucleotide.

91. The conjugate compound of embodiment 76-90, which display improved cellular distribution between liver vs. kidney or improved cellular uptake into the liver of the conjugate compound as compared to an unconjugated nucleic acid molecule or antisense oligonucleotide.

92. The conjugate compound of embodiment 76-91, where in the conjugate compound is selected from the group consisting of CPM ID NO 20_12, 20_13, 20_14, 20_15, 20_16, 20_18, 2020, 20_21, 20_22, 20_30, 20_35, 20_36, 212, 21_33 and 21_34.

93. A pharmaceutical composition comprising a nucleic acid molecule according to any one of embodiments 1 to 11, an antisense oligonucleotide of embodiment 12-73, a conjugate compound of embodiment 74-92 or acceptable salts thereof and a pharmaceutically acceptable diluent, carrier, salt and/or adjuvant.

94. A method for manufacturing the antisense oligonucleotide of embodiment 12-73, comprising reacting nucleotide units thereby forming covalently linked contiguous nucleotide units comprised in the antisense oligonucleotide.

95. The method of embodiment 94, further comprising reacting the contiguous nucleotide sequence with a non-nucleotide conjugation moiety as described in any one of claims 76-84.

96. A method for manufacturing the composition of embodiment 93, comprising mixing the antisense oligonucleotide with a pharmaceutically acceptable diluent, carrier, salt and/or adjuvant.

97. An in vivo or in vitro method for modulating PAPD5 and PAPD7 expression in a target cell which is expressing PAPD5 and PAPD7, said method comprising administering the nucleic acid molecule of any one of embodiments 1 to 11, the antisense oligonucleotide of any one of embodiments 12-73 or the conjugate compound of any one of embodiment 74-92 or the pharmaceutical composition of embodiment 93 in an effective amount to said cell.

98. The method of embodiments 97, wherein the PAPD5 and PAPD7 expression is reduced by at least 30%, or at least or at least 40%, or at least 50%, or at least 60%, or at least 70%, or at least 80%, or at least 90%, or at least 95% in the target cell compared to the level without any treatment.

99. A method for treating or preventing a disease comprising administering a therapeutically or prophylactically effective amount of the nucleic acid molecule any one of embodiments 1 to 11, the antisense oligonucleotide of any one of embodiments 12-73 or the conjugate compound of any one of embodiments 74-92 or the pharmaceutical composition of embodiment 93 to a subject suffering from or susceptible to the disease.

100. The nucleic acid molecule any one of embodiments 1 to 11, the antisense oligonucleotide of any one of embodiments 12-57 or the conjugate compound of any one of embodiments 74-92 or the pharmaceutical composition of embodiment 93, for use as a medicament for treatment or prevention of a disease in a subject.

101. Use of the nucleic acid molecule any one of embodiments 1 to 11, the antisense oligonucleotide of any one of embodiment 12-73 or the conjugate compound of any one of embodiment 74-92 for the preparation of a medicament for treatment or prevention of a disease in a subject.

102. The method, the nucleic acid molecule, or the use of embodiments 99-101, wherein the disease is HBV infection or chronic HBV infection.

103. The method, the nucleic acid molecule or the use of embodiments 102, wherein the secretion of HBsAg and/or HBeAg and/or intracellular HBV mRNA and/or HBV DNA is reduced.

104. The method, the nucleic acid molecule or the use of embodiments 102 or 103, wherein HBsAg is reduced by at least 30%, or at least or at least 40%, or at least 50%, or at least 60%, or at least 70%, or at least 80%, or at least 90%, or at least 95% compared to the level without any treatment.

105. The method, the antisense oligonucleotide or the use of embodiments 99-104 wherein the subject is a mammal.

106. The method, the antisense oligonucleotide or the use of embodiment 105, wherein the mammal is human.

### EXAMPLES

The Examples illustrate the invention.

### Material and Methods

### Oligonucleotide Motif Sequences and Oligonucleotide Compounds

TABLE 4
List of oligonucleotide motif sequences targeting
human and mouse transcripts
Sequences are indicated by SEQ ID NO, the motif
sequence and the position they target on the human
PAPD5 transcript (SEQ ID NO: 1) and the human
PAPD7 transcript (SEQ ID NO: 2).

| SEQ | | Start | End | Start | End |
|---|---|---|---|---|---|
| ID | | ID | ID | ID | ID |
| NO | Motif Sequence | NO: 1 | NO: 1 | NO: 2 | NO: 2 |
| 7 | AGATCTGCATCCACAG | 759 | 774 | 1032 | 1047 |
| 8 | CAGATCTGCATCCACAG | 759 | 775 | 1032 | 1048 |
| 9 | CCAGATCTGCATCCACAG | 759 | 776 | 1032 | 1040 |
| 10 | CCAGATCTGCATCCACA | 760 | 776 | 1033 | 1049 |
| 11 | CCCAGATCTGCATCCAC | 761 | 777 | 1034 | 1050 |
| 12 | CCCAGATCTGCATCCA | 762 | 777 | 1035 | 1050 |
| 13 | TCCCAGATCTGCATCCA | 762 | 778 | 1035 | 1051 |
| 14 | GTCTCCCAGATCTGCAT | 765 | 781 | 1038 | 1054 |
| 15 | TCTCCCAGATCTGCAT | 765 | 780 | 1038 | 1053 |
| 16 | GTCTCCCAGATCTGCA | 766 | 781 | 1039 | 1054 |

Motif sequences represent the contiguous sequence of nucleobases present in the oligonucleotide.

TABLE 5
Lists oligonucleotides designs and specific antisense
oligonucleotide compounds Compounds are indicated
by CMP ID NO, and based on the on the motif sequence in table 4.

| SEQ | | | | |
|---|---|---|---|---|
| ID NO | Design | Oligonucleotide Compound | CMP ID NO | dG |
| 7 | 1-1-1-7-3-1-2 | AgAtctgcatCCAcAG | 7_1 | −23 |
| 7 | 1-9-3-1-2 | AgatctgcatCCAcAG | 7_2 | −22 |
| 7 | 1-9-2-1-3 | AgatctgcatCCaCAG | 7_3 | −23 |
| 7 | 1-1-2-6-2-2-2 | AgATctgcatCCacAG | 7_4 | −23 |
| 7 | 1-1-1-7-2-2-2 | AgAtctgcatCCacAG | 7_5 | −21 |
| 7 | 1-3-1-5-2-2-2 | AgatCtgcatCCacAG | 7_6 | −22 |
| 7 | 1-9-2-2-2 | AgatctgcatCCacAG | 7_7 | −21 |
| 7 | 2-8-1-1-4 | AGatctgcatCcACAG | 7_8 | −23 |
| 7 | 1-1-1-7-1-1-4 | AgAtctgcatCcACAG | 7_9 | −22 |
| 7 | 1-3-1-5-1-1-4 | AgatCtgcatCcACAG | 7_10 | −22 |
| 7 | 1-9-1-1-4 | AgatctgcatCcACAG | 7_11 | −21 |
| 7 | 3-7-1-1-1-1-2 | AGAtctgcatCcAcAG | 7_12 | −22 |
| 7 | 2-2-1-5-1-1-1-1-2 | AGatCtgcatCcAcAG | 7_13 | −21 |
| 7 | 2-8-1-1-1-1-2 | AGatctgcatCcAcAG | 7_14 | −20 |
| 7 | 1-1-3-5-1-1-1-1-2 | AgATCtgcatCcAcAG | 7_15 | −22 |
| 7 | 1-1-1-1-1-5-1-1-1-1-2 | AgAtCtgcatCcAcAG | 7_16 | −20 |
| 7 | 1-1-1-7-1-1-1-1-2 | AgAtctgcatCcAcAG | 7_17 | −19 |
| 7 | 1-2-2-5-1-1-1-1-2 | AgaTCtgcatCcAcAG | 7_18 | −21 |
| 7 | 1-3-1-5-1-1-1-1-2 | AgatCtgcatCcAcAG | 7_19 | −20 |
| 7 | 1-9-1-1-1-1-2 | AgatctgcatCcAcAG | 7_20 | −19 |
| 7 | 1-1-2-6-1-2-3 | AgATctgcatCcaCAG | 7_21 | −23 |
| 7 | 1-1-1-7-1-2-3 | AgAtctgcatCcaCAG | 7_22 | −21 |
| 7 | 1-3-1-5-1-2-3 | AgatCtgcatCcaCAG | 7_23 | −22 |
| 7 | 1-9-1-2-3 | AgatctgcatCcaCAG | 7_24 | −21 |
| 7 | 3-7-1-3-2 | AGAtctgcatCcacAG | 7_25 | −22 |
| 7 | 2-2-1-5-1-3-2 | AGatCtgcatCcacAG | 7_26 | −21 |
| 7 | 2-8-1-3-2 | AGatctgcatCcacAG | 7_27 | −20 |
| 7 | 1-1-3-5-1-3-2 | AgATCtgcatCcacAG | 7_28 | −22 |
| 7 | 1-1-1-1-1-5-1-3-2 | AgAtCtgcatCcacAG | 7_29 | −20 |
| 7 | 1-1-1-7-1-3-2 | AgAtctgcatCcacAG | 7_30 | −19 |
| 7 | 1-2-2-5-1-3-2 | AgaTCtgcatCcacAG | 7_31 | −21 |
| 7 | 1-3-1-5-1-3-2 | AgatCtgcatCcacAG | 7_32 | −20 |
| 7 | 1-9-1-3-2 | AgatctgcatCcacAG | 7_33 | −19 |
| 7 | 1-1-1-8-5 | AgAtctgcatcCACAG | 7_34 | −23 |
| 7 | 1-10-5 | AgatctgcatcCACAG | 7_35 | −23 |
| 7 | 2-2-1-6-2-1-2 | AGatCtgcatcCAcAG | 7_36 | −22 |
| 7 | 2-9-2-1-2 | AGatctgcatcCAcAG | 7_37 | −21 |
| 7 | 1-1-2-7-2-1-2 | AgATctgcatcCAcAG | 7_38 | −22 |
| 7 | 1-1-1-1-1-6-2-1-2 | AgAtCtgcatcCAcAG | 7_39 | −22 |
| 7 | 1-1-1-8-2-1-2 | AgAtctgcatcCAcAG | 7_40 | −21 |
| 7 | 1-3-1-6-2-1-2 | AgatCtgcatcCAcAG | 7_41 | −21 |
| 7 | 1-10-2-1-2 | AgatctgcatcCAcAG | 7_42 | −20 |
| 7 | 1-1-1-8-1-1-3 | AgAtctgcatcCaCAG | 7_43 | −21 |
| 7 | 1-3-1-6-1-1-3 | AgatCtgcatcCaCAG | 7_44 | −22 |
| 7 | 1-10-1-1-3 | AgatctgcatcCaCAG | 7_45 | −21 |
| 7 | 3-1-1-6-1-2-2 | AGAtCtgcatcCacAG | 7_46 | −22 |
| 7 | 2-2-1-6-1-2-2 | AGatCtgcatcCacAG | 7_47 | −21 |
| 7 | 1-1-3-6-1-2-2 | AgATCtgcatcCacAG | 7_48 | −22 |
| 7 | 1-1-1-1-1-6-1-2-2 | AgAtCtgcatcCacAG | 7_49 | −20 |
| 7 | 1-1-1-8-1-2-2 | AgAtctgcatcCacAG | 7_50 | −19 |
| 7 | 1-2-2-6-1-2-2 | AgaTCtgcatcCacAG | 7_51 | −21 |
| 7 | 1-3-1-6-1-2-2 | AgatCtgcatcCacAG | 7_52 | −20 |
| 7 | 1-10-1-2-2 | AgatctgcatcCacAG | 7_53 | −19 |
| 7 | 1-1-1-1-1-7-4 | AgAtCtgcatccACAG | 7_54 | −22 |
| 7 | 1-1-1-9-4 | AgAtctgcatccACAG | 7_55 | −21 |
| 7 | 1-2-2-7-4 | AgaTCtgcatccACAG | 7_56 | −23 |
| 7 | 1-3-1-7-4 | AgatCtgcatccACAG | 7_57 | −22 |
| 7 | 1-11-4 | AgatctgcatccACAG | 7_58 | −21 |
| 7 | 3-1-1-7-1-1-2 | AGAtCtgcatccAcAG | 7_59 | −22 |
| 7 | 3-9-1-1-2 | AGAtctgcatccAcAG | 7_60 | −21 |
| 7 | 2-2-1-7-1-1-2 | AGatCtgcatccAcAG | 7_61 | −20 |
| 7 | 1-1-3-7-1-1-2 | AgATCtgcatccAcAG | 7_62 | −22 |
| 7 | 1-1-1-1-1-7-1-1-2 | AgAtCtgcatccAcAG | 7_63 | −20 |
| 7 | 1-1-1-9-1-1-2 | AgAtctgcatccAcAG | 7_64 | −19 |
| 7 | 1-2-2-7-1-1-2 | AgaTCtgcatccAcAG | 7_65 | −20 |
| 7 | 1-3-1-7-1-1-2 | AgatCtgcatccAcAG | 7_66 | −19 |
| 7 | 1-11-1-1-2 | AgatctgcatccAcAG | 7_67 | −18 |
| 7 | 3-10-3 | AGAtctgcatccaCAG | 7_68 | −23 |
| 7 | 1-1-1-1-1-8-3 | AgAtCtgcatccaCAG | 7_69 | −22 |
| 7 | 1-1-1-10-3 | AgAtctgcatccaCAG | 7_70 | −21 |
| 7 | 1-2-2-8-3 | AgaTCtgcatccaCAG | 7_71 | −22 |
| 7 | 1-3-1-8-3 | AgatCtgcatccaCAG | 7_72 | −21 |
| 7 | 1-12-3 | AgatctgcatccaCAG | 7_73 | −20 |
| 7 | 3-1-1-9-2 | AGAtCtgcatccacAG | 7_74 | −22 |
| 7 | 3-11-2 | AGAtctgcatccacAG | 7_75 | −21 |
| 7 | 2-1-2-9-2 | AGaTCtgcatccacAG | 7_76 | −21 |
| 7 | 2-2-1-9-2 | AGatCtgcatccacAG | 7_77 | −20 |
| 7 | 1-1-3-9-2 | AgATCtgcatccacAG | 7_78 | −21 |
| 7 | 1-1-1-1-1-9-2 | AgAtCtgcatccacAG | 7_79 | −19 |
| 7 | 1-1-1-11-2 | AgAtctgcatccacAG | 7_80 | −18 |
| 7 | 1-2-2-9-2 | AgaTCtgcatccacAG | 7_81 | −20 |
| 7 | 1-3-1-9-2 | AgatCtgcatccacAG | 7_82 | −19 |
| 7 | 1-13-2 | AgatctgcatccacAG | 7_83 | −18 |
| 8 | 1-2-1-7-2-2-2 | CagAtctgcatCCacAG | 8_1 | −23 |
| 8 | 1-3-1-6-2-2-2 | CagaTctgcatCCacAG | 8_2 | −23 |
| 8 | 1-10-2-2-2 | CagatctgcatCCacAG | 8_3 | −22 |
| 8 | 1-2-1-7-1-1-4 | CagAtctgcatCcACAG | 8_4 | −23 |
| 8 | 1-10-1-1-4 | CagatctgcatCcACAG | 8_5 | −23 |
| 8 | 2-1-1-7-1-1-1-1-2 | CAgAtctgcatCcAcAG | 8_6 | −23 |
| 8 | 2-3-1-5-1-1-1-1-2 | CAgatCtgcatCcAcAG | 8_7 | −23 |
| 8 | 2-9-1-1-1-1-2 | CAgatctgcatCcAcAG | 8_8 | −22 |
| 8 | 1-1-2-7-1-1-1-1-2 | CaGAtctgcatCcAcAG | 8_9 | −23 |
| 8 | 1-1-1-2-1-5-1-1-1-1-2 | CaGatCtgcatCcAcAG | 8_10 | −22 |
| 8 | 1-1-1-8-1-1-1-1-2 | CaGatctgcatCcAcAG | 8_11 | −21 |
| 8 | 1-2-1-1-1-5-1-1-1-1-2 | CagAtCtgcatCcAcAG | 8_12 | −22 |
| 8 | 1-2-1-7-1-1-1-1-2 | CagAtctgcatCcAcAG | 8_13 | −21 |
| 8 | 1-3-2-5-1-1-1-1-2 | CagaTCtgcatCcAcAG | 8_14 | −22 |
| 8 | 1-4-1-5-1-1-1-1-2 | CagatCtgcatCcAcAG | 8_15 | −21 |
| 8 | 1-10-1-1-1-1-2 | CagatctgcatCcAcAG | 8_16 | −20 |
| 8 | 1-2-1-7-1-2-3 | CagAtctgcatCcaCAG | 8_17 | −23 |
| 8 | 1-10-1-2-3 | CagatctgcatCcaCAG | 8_18 | −22 |
| 8 | 2-1-1-7-1-3-2 | CAgAtctgcatCcacAG | 8_19 | −23 |
| 8 | 2-3-1-5-1-3-2 | CAgatCtgcatCcacAG | 8_20 | −23 |
| 8 | 2-9-1-3-2 | CAgatctgcatCcacAG | 8_21 | −22 |
| 8 | 1-1-2-7-1-3-2 | CaGAtctgcatCcacAG | 8_22 | −23 |
| 8 | 1-1-1-2-1-5-1-3-2 | CaGatCtgcatCcacAG | 8_23 | −22 |
| 8 | 1-1-1-8-1-3-2 | CaGatctgcatCcacAG | 8_24 | −21 |
| 8 | 1-2-1-1-1-5-1-3-2 | CagAtCtgcatCcacAG | 8_25 | −22 |
| 8 | 1-2-1-7-1-3-2 | CagAtctgcatCcacAG | 8_26 | −21 |
| 8 | 1-3-2-5-1-3-2 | CagaTCtgcatCcacAG | 8_27 | −22 |
| 8 | 1-4-1-5-1-3-2 | CagatCtgcatCcacAG | 8_28 | −21 |
| 8 | 1-10-1-3-2 | CagatctgcatCcacAG | 8_29 | −20 |
| 8 | 1-2-1-8-5 | CagAtctgcatcCACAG | 8_30 | −24 |
| 8 | 1-2-1-1-1-6-2-1-2 | CagAtCtgcatcCAcAG | 8_31 | −23 |
| 8 | 1-2-1-8-2-1-2 | CagAtctgcatcCAcAG | 8_32 | −22 |
| 8 | 1-4-1-6-2-1-2 | CagatCtgcatcCAcAG | 8_33 | −22 |
| 8 | 1-11-2-1-2 | CagatctgcatcCAcAG | 8_34 | −21 |
| 8 | 1-2-1-8-1-1-3 | CagAtctgcatcCaCAG | 8_35 | −22 |
| 8 | 1-4-1-6-1-1-3 | CagatCtgcatcCaCAG | 8_36 | −23 |
| 8 | 1-11-1-1-3 | CagatctgcatcCaCAG | 8_37 | −22 |
| 8 | 2-1-1-8-1-2-2 | CAgAtctgcatcCacAG | 8_38 | −22 |
| 8 | 2-3-1-6-1-2-2 | CAgatCtgcatcCacAG | 8_39 | −23 |
| 8 | 2-10-1-2-2 | CAgatctgcatcCacAG | 8_40 | −22 |
| 8 | 1-1-2-1-1-6-1-2-2 | CaGAtCtgcatcCacAG | 8_41 | −23 |
| 8 | 1-1-1-2-1-6-1-2-2 | CaGatCtgcatcCacAG | 8_42 | −22 |
| 8 | 1-2-3-6-1-2-2 | CagATCtgcatcCacAG | 8_43 | −23 |
| 8 | 1-2-1-1-1-6-1-2-2 | CagAtCtgcatcCacAG | 8_44 | −21 |
| 8 | 1-2-1-8-1-2-2 | CagAtctgcatcCacAG | 8_45 | −20 |
| 8 | 1-3-2-6-1-2-2 | CagaTCtgcatcCacAG | 8_46 | −22 |
| 8 | 1-4-1-6-1-2-2 | CagatCtgcatcCacAG | 8_47 | −21 |
| 8 | 1-11-1-2-2 | CagatctgcatcCacAG | 8_48 | −20 |
| 8 | 2-1-1-9-4 | CAgAtctgcatccACAG | 8_49 | −24 |
| 8 | 1-2-1-1-1-7-4 | CagAtCtgcatccACAG | 8_50 | −23 |
| 8 | 1-4-1-7-4 | CagatCtgcatccACAG | 8_51 | −23 |
| 8 | 1-12-4 | CagatctgcatccACAG | 8_52 | −22 |
| 8 | 2-1-1-1-1-7-1-1-2 | CAgAtCtgcatccAcAG | 8_53 | −23 |
| 8 | 2-1-1-9-1-1-2 | CAgAtctgcatccAcAG | 8_54 | −22 |
| 8 | 2-3-1-7-1-1-2 | CAgatCtgcatccAcAG | 8_55 | −22 |
| 8 | 2-11-1-1-2 | CAgatctgcatccAcAG | 8_56 | −21 |
| 8 | 1-1-2-1-1-7-1-1-2 | CaGAtCtgcatccAcAG | 8_57 | −23 |
| 8 | 1-1-1-2-1-7-1-1-2 | CaGatCtgcatccAcAG | 8_58 | −21 |
| 8 | 1-2-3-7-1-1-2 | CagATCtgcatccAcAG | 8_59 | −23 |
| 8 | 1-2-1-1-1-7-1-1-2 | CagAtCtgcatccAcAG | 8_60 | −21 |
| 8 | 1-2-1-9-1-1-2 | CagAtctgcatccAcAG | 8_61 | −20 |
| 8 | 1-3-2-7-1-1-2 | CagaTCtgcatccAcAG | 8_62 | −22 |
| 8 | 1-4-1-7-1-1-2 | CagatCtgcatccAcAG | 8_63 | −20 |
| 8 | 1-12-1-1-2 | CagatctgcatccAcAG | 8_64 | −19 |
| 8 | 2-1-1-10-3 | CAgAtctgcatccaCAG | 8_65 | −24 |
| 8 | 1-2-1-1-1-8-3 | CagAtCtgcatccaCAG | 8_66 | −23 |
| 8 | 1-2-1-10-3 | CagAtctgcatccaCAG | 8_67 | −22 |
| 8 | 1-4-1-8-3 | CagatCtgcatccaCAG | 8_68 | −22 |
| 8 | 1-13-3 | CagatctgcatccaCAG | 8_69 | −21 |
| 8 | 2-1-1-1-1-9-2 | CAgAtCtgcatccacAG | 8_70 | −23 |
| 8 | 2-1-1-11-2 | CAgAtctgcatccacAG | 8_71 | −22 |
| 8 | 2-2-2-9-2 | CAgaTCtgcatccacAG | 8_72 | −23 |
| 8 | 2-3-1-9-2 | CAgatCtgcatccacAG | 8_73 | −22 |
| 8 | 2-13-2 | CAgatctgcatccacAG | 8_74 | −21 |
| 8 | 1-1-2-1-1-9-2 | CaGAtCtgcatccacAG | 8_75 | −23 |
| 8 | 1-1-1-2-1-9-2 | CaGatCtgcatccacAG | 8_76 | −21 |
| 8 | 1-2-1-1-1-9-2 | CagAtCtgcatccacAG | 8_77 | −21 |
| 8 | 1-2-1-11-2 | CagAtctgcatccacAG | 8_78 | −20 |
| 8 | 1-3-2-9-2 | CagaTCtgcatccacAG | 8_79 | −21 |
| 8 | 1-4-1-9-2 | CagatCtgcatccacAG | 8_80 | −20 |
| 8 | 1-14-2 | CagatctgcatccacAG | 8_81 | −19 |
| 9 | 1-3-1-7-1-1-1-1-2 | CcagAtctgcatCcAcAG | 9_1 | −24 |
| 9 | 1-1-1-1-1-7-1-3-2 | CcAgAtctgcatCcacAG | 9_2 | −24 |
| 9 | 1-1-1-10-1-2-2 | CcAgatctgcatcCacAG | 9_3 | −23 |
| 9 | 1-12-1-2-2 | CcagatctgcatcCacAG | 9_4 | −23 |
| 9 | 1-1-1-1-1-9-1-1-2 | CcAgAtctgcatccAcAG | 9_5 | −23 |
| 9 | 1-1-1-11-1-1-2 | CcAgatctgcatccAcAG | 9_6 | −23 |
| 9 | 1-3-1-9-1-1-2 | CcagAtctgcatccAcAG | 9_7 | −23 |
| 9 | 1-13-1-1-2 | CcagatctgcatccAcAG | 9_8 | −22 |
| 9 | 1-3-1-10-3 | CcagAtctgcatccaCAG | 9_9 | −25 |
| 9 | 2-2-1-11-2 | CCagAtctgcatccacAG | 9_10 | −25 |
| 9 | 1-1-1-13-2 | CcAgatctgcatccacAG | 9_11 | −23 |
| 9 | 1-2-2-11-2 | CcaGAtctgcatccacAG | 9_12 | −25 |
| 10 | 1-3-1-6-1-3-2 | CcagAtctgcaTccaCA | 10_1 | −23 |
| 10 | 1-3-1-7-1-1-3 | CcagAtctgcatCcACA | 10_2 | −24 |
| 10 | 1-1-1-9-1-2-2 | CcAgatctgcatCcaCA | 10_3 | −23 |
| 10 | 1-3-1-7-1-2-2 | CcagAtctgcatCcaCA | 10_4 | −23 |
| 10 | 1-11-1-2-2 | CcagatctgcatCcaCA | 10_5 | −23 |
| 10 | 1-3-1-8-4 | CcagAtctgcatcCACA | 10_6 | −25 |
| 10 | 1-1-1-10-1-1-2 | CcAgatctgcatcCaCA | 10_7 | −23 |
| 10 | 1-3-1-8-1-1-2 | CcagAtctgcatcCaCA | 10_8 | −23 |
| 10 | 1-12-1-1-2 | CcagatctgcatcCaCA | 10_9 | −22 |
| 10 | 1-1-1-1-1-9-3 | CcAgAtctgcatccACA | 10_10 | −23 |
| 10 | 1-1-1-11-3 | CcAgatctgcatccACA | 10_11 | −23 |
| 10 | 1-3-1-9-3 | CcagAtctgcatccACA | 10_12 | −23 |
| 10 | 1-13-3 | CcagatctgcatccACA | 10_13 | −22 |
| 10 | 1-1-1-1-1-10-2 | CcAgAtctgcatccaCA | 10_14 | −23 |
| 10 | 1-1-1-12-2 | CcAgatctgcatccaCA | 10_15 | −22 |
| 10 | 1-2-2-10-2 | CcaGAtctgcatccaCA | 10_16 | −24 |
| 10 | 1-3-1-10-2 | CcagAtctgcatccaCA | 10_17 | −22 |
| 10 | 1-14-2 | CcagatctgcatccaCA | 10_18 | −22 |
| 11 | 1-1-1-8-1-1-1-1-2 | CcCagatctgcAtCcAC | 11_1 | −23 |
| 11 | 1-2-1-7-1-1-1-1-2 | CccAgatctgcAtCcAC | 11_2 | −23 |
| 11 | 1-10-1-1-1-1-2 | CccagatctgcAtCcAC | 11_3 | −23 |
| 11 | 1-1-1-8-1-2-3 | CcCagatctgcAtcCAC | 11_4 | −25 |
| 11 | 1-2-1-7-1-2-3 | CccAgatctgcAtcCAC | 11_5 | −25 |
| 11 | 1-10-1-2-3 | CccagatctgcAtcCAC | 11_6 | −24 |
| 11 | 2-1-1-7-1-3-2 | CCcAgatctgcAtccAC | 11_7 | −25 |
| 11 | 2-9-1-3-2 | CCcagatctgcAtccAC | 11_8 | −24 |
| 11 | 1-1-2-7-1-3-2 | CcCAgatctgcAtccAC | 11_9 | −25 |
| 11 | 1-1-1-1-1-6-1-3-2 | CcCaGatctgcAtccAC | 11_10 | −23 |
| 11 | 1-1-1-8-1-3-2 | CcCagatctgcAtccAC | 11_11 | −23 |
| 11 | 1-2-2-6-1-3-2 | CccAGatctgcAtccAC | 11_12 | −24 |
| 11 | 1-2-1-1-1-5-1-3-2 | CccAgAtctgcAtccAC | 11_13 | −23 |
| 11 | 1-2-1-7-1-3-2 | CccAgatctgcAtccAC | 11_14 | −23 |
| 11 | 1-10-1-3-2 | CccagatctgcAtccAC | 11_15 | −22 |
| 11 | 1-2-1-1-1-7-1-1-2 | CccAgAtctgcatCcAC | 11_16 | −24 |
| 11 | 1-12-1-1-2 | CccagatctgcatCcAC | 11_17 | −23 |
| 11 | 1-2-1-1-1-8-3 | CccAgAtctgcatcCAC | 11_18 | −25 |
| 11 | 1-4-1-8-3 | CccagAtctgcatcCAC | 11_19 | −24 |
| 11 | 2-3-1-9-2 | CCcagAtctgcatccAC | 11_20 | −25 |
| 11 | 1-1-2-1-1-9-2 | CcCAgAtctgcatccAC | 11_21 | −25 |
| 11 | 1-1-1-1-2-9-2 | CcCaGAtctgcatccAC | 11_22 | −25 |
| 11 | 1-1-1-12-2 | CcCagatctgcatccAC | 11_23 | −23 |
| 11 | 1-2-1-1-1-9-2 | CccAgAtctgcatccAC | 11_24 | −23 |
| 11 | 1-2-1-11-2 | CccAgatctgcatccAC | 11_25 | −23 |
| 11 | 1-14-2 | CccagatctgcatccAC | 11_26 | −22 |
| 12 | 1-9-2-2-2 | CccagatctgCAtcCA | 12_1 | −24 |
| 12 | 1-1-1-7-1-3-2 | CcCagatctgCatcCA | 12_2 | −23 |
| 12 | 1-2-1-6-1-3-2 | CccAgatctgCatcCA | 12_3 | −23 |
| 12 | 1-9-1-3-2 | CccagatctgCatcCA | 12_4 | −23 |
| 12 | 1-2-1-7-1-1-3 | CccAgatctgcAtCCA | 12_5 | −25 |
| 12 | 1-10-1-1-3 | CccagatctgcAtCCA | 12_6 | −24 |
| 12 | 2-9-1-2-2 | CCcagatctgcAtcCA | 12_7 | −24 |
| 12 | 1-1-1-8-1-2-2 | CcCagatctgcAtcCA | 12_8 | −23 |
| 12 | 1-2-1-7-1-2-2 | CccAgatctgcAtcCA | 12_9 | −23 |
| 12 | 1-3-1-6-1-2-2 | CccaGatctgcAtcCA | 12_10 | −23 |
| 12 | 1-10-1-2-2 | CccagatctgcAtcCA | 12_11 | −22 |
| 12 | 2-1-1-10-2 | CCcAgatctgcatcCA | 12_12 | −25 |
| 12 | 1-1-1-11-2 | CcCagatctgcatcCA | 12_13 | −22 |
| 12 | 1-2-1-10-2 | CccAgatctgcatcCA | 12_14 | −22 |
| 12 | 1-13-2 | CccagatctgcatcCA | 12_15 | −22 |
| 13 | 2-10-1-2-2 | TCccagatctgcAtcCA | 13_1 | −24 |
| 13 | 2-2-1-10-2 | TCccAgatctgcatcCA | 13_2 | −25 |
| 14 | 1-3-1-6-1-1-1-1-2 | GtctCccagatCtGcAT | 14_1 | −24 |
| 14 | 1-4-1-5-1-3-2 | GtctcCcagatCtgcAT | 14_2 | −23 |
| 14 | 1-10-1-3-2 | GtctcccagatCtgcAT | 14_3 | −23 |
| 14 | 1-1-1-2-1-6-1-2-2 | GtCtcCcagatcTgcAT | 14_4 | −24 |
| 14 | 1-4-1-6-1-2-2 | GtctcCcagatcTgcAT | 14_5 | −23 |
| 14 | 1-1-1-1-1-8-1-1-2 | GtCtCccagatctGcAT | 14_6 | −24 |
| 14 | 1-2-2-8-1-1-2 | GtcTCccagatctGcAT | 14_7 | −24 |
| 14 | 1-4-1-7-1-1-2 | GtctcCcagatctGcAT | 14_8 | −23 |
| 14 | 1-4-1-8-3 | GtctcCcagatctgCAT | 14_9 | −25 |
| 14 | 1-1-1-2-1-9-2 | GtCtcCcagatctgcAT | 14_10 | −23 |
| 14 | 1-1-1-12-2 | GtCtcccagatctgcAT | 14_11 | −23 |
| 14 | 1-3-1-10-2 | GtctCccagatctgcAT | 14_12 | −22 |
| 14 | 1-4-1-9-2 | GtctcCcagatctgcAT | 14_13 | −22 |
| 15 | 2-8-1-1-1-1-2 | TCtcccagatCtGcAT | 15_1 | −22 |
| 15 | 1-3-1-5-1-2-3 | TctcCcagatCtgCAT | 15_2 | −23 |
| 15 | 2-1-1-6-1-3-2 | TCtCccagatCtgcAT | 15_3 | −23 |
| 15 | 2-2-1-5-1-3-2 | TCtcCcagatCtgcAT | 15_4 | −23 |
| 15 | 2-8-1-3-2 | TCtcccagatCtgcAT | 15_5 | −22 |
| 15 | 1-3-1-5-1-3-2 | TctcCcagatCtgcAT | 15_6 | −21 |
| 15 | 2-9-2-1-2 | TCtcccagatcTGcAT | 15_7 | −23 |
| 15 | 2-1-1-7-1-2-2 | TCtCccagatcTgcAT | 15_8 | −23 |
| 15 | 2-2-1-6-1-2-2 | TCtcCcagatcTgcAT | 15_9 | −23 |
| 15 | 2-9-1-2-2 | TCtcccagatcTgcAT | 15_10 | −22 |
| 15 | 4-8-1-1-2 | TCTCccagatctGcAT | 15_11 | −24 |
| 15 | 3-9-1-1-2 | TCTcccagatctGcAT | 15_12 | −23 |
| 15 | 2-2-1-7-1-1-2 | TCtcCcagatctGcAT | 15_13 | −22 |
| 15 | 2-10-1-1-2 | TCtcccagatctGcAT | 15_14 | −21 |
| 15 | 2-2-1-8-3 | TCtcCcagatctgCAT | 15_15 | −24 |
| 15 | 1-3-1-8-3 | TctcCcagatctgCAT | 15_16 | −22 |
| 15 | 3-11-2 | TCtcccagatctgcAT | 15_17 | −22 |
| 15 | 2-1-1-10-2 | TCtCccagatctgcAT | 15_18 | −22 |
| 15 | 2-2-1-9-2 | TCtcCcagatctgcAT | 15_19 | −22 |
| 15 | 2-12-2 | TCtcccagatctgcAT | 15_20 | −21 |
| 15 | 1-2-2-9-2 | TctCCcagatctgcAT | 15_21 | −23 |
| 16 | 1-3-1-6-1-2-2 | GtctCccagatCtgCA | 16_1 | −24 |
| 16 | 1-10-1-2-2 | GtctcccagatCtgCA | 16_2 | −23 |
| 16 | 1-1-1-1-1-9-2 | GtCtCccagatctgCA | 16_3 | −24 |
| 16 | 1-1-1-11-2 | GtCtcccagatctgCA | 16_4 | −23 |
| 16 | 1-3-1-9-2 | GtctCccagatctgCA | 16_5 | −23 |

Designs refer to the gapmer design, F-G-F′. In classic gapmer design e.g. 3-10-3 all the nucleotides in the flanks (F and F′) are constituted of the same 2′-sugar modified nucleoside, e.g. LNA, cET, or MOE, and a stretch of DNA in the middle forming the gap (G). In gapmers with alternating flank designs the flanks of oligonucleotide is annotated as a series of integers, representing a number of 2′ sugar modified nucleosides (M) followed by a number of DNA nucleosides (D). For example a flank with a 2-2-1 motif represents 5′ [M]₂-[D]₂-[M]3′ and a 1-1-1-1-1 motif represents 5′ [M]-[D]-[M]-[D]-[M]3′. Both flanks have a 2′ sugar modified nucleoside at the 5′ and 3′ terminal. The gap region (G), which is constituted of a number of DNA nucleosides (typically between 5 and 16), is located between the flanks.

The heading “Oligonucleotide compound” in the table represents specific designs of a motifsequence. Capital letters represent beta-D-oxy LNA nucleosides, lowercase letters represent DNA nucleosides, all LNA C are 5-methyl cytosine, and 5-methyl cytosine DNA are presented by “e”, all internucleoside linkages are phosphorothioate internucleoside linkages.

TABLE 6
list of oligonucleotide motif sequences targeting
human and cyno Sequences are indicated by SEQ
ID NO, the motif sequence (nucleobase sequence)
and the position they target on the human PAPD5
transcript (SEQ ID NO: 1) and the human PAPD7
transcript (SEQ ID NO: 2).

| | | Start | | Start | |
|---|---|---|---|---|---|
| SEQ | | ID | End ID | ID | End ID |
| ID NO | Motif Sequence | NO: 1 | NO: 1 | NO: 2 | NO: 2 |
| 17 | TCAACTTTCACTTCAGT | 64669 | 64685 | 29514 | 29530 |
| 18 | TCAACTTTCACTTCAG | 64670 | 64685 | 29515 | 29530 |
| 19 | TGTTTCAATACTAAAA | 69414 | 69429 | 30731 | 30746 |

Motif sequences represent the contiguous sequence of nucleobases present in the oligonucleotide.

TABLE 7
Lists oligonucleotides designs and specific antisense
oligonucleotide compounds Compounds are indicated
by CMP ID NO, and based on the on the motif sequence in table 6.

| SEQ | | | | |
|---|---|---|---|---|
| ID NO | Design | Oligonucleotide Compound | CMP ID NO | dG |
| 17 | 2-12-3 | TCaactttcacttcAGT | 17_1 | −19 |
| 17 | 2-2-1-6-1-2-3 | TCaaCtttcacTtcAGT | 17_2 | −21 |
| 17 | 2-9-1-2-3 | TCaactttcacTtcAGT | 17_3 | −20 |
| 17 | 1-3-1-6-1-2-3 | TcaaCtttcacTtcAGT | 17_4 | −20 |
| 17 | 2-9-1-3-2 | TCaactttcacTtcaGT | 17_5 | −19 |
| 17 | 2-2-1-7-2-1-2 | TCaaCtttcactTCaGT | 17_6 | −21 |
| 17 | 1-1-1-9-1-1-3 | TcAactttcactTcAGT | 17_7 | −19 |
| 17 | 1-1-2-8-1-2-2 | TcAActttcactTcaGT | 17_8 | −18 |
| 17 | 5-8-1-1-2 | TCAACtttcacttCaGT | 17_9 | −23 |
| 17 | 4-9-1-1-2 | TCAActttcacttCaGT | 17_10 | −21 |
| 17 | 2-2-1-8-1-1-2 | TCaaCtttcacttCaGT | 17_11 | −20 |
| 17 | 2-11-1-1-2 | TCaactttcacttCaGT | 17_12 | −19 |
| 17 | 1-1-2-9-1-1-2 | TcAActttcacttCaGT | 17_13 | −18 |
| 17 | 3-11-3 | TCAactttcacttcAGT | 17_14 | −21 |
| 17 | 2-2-1-9-3 | TCaaCtttcacttcAGT | 17_15 | −20 |
| 17 | 2-13-2 | TCaactttcacttcaGT | 17_16 | −18 |
| 17 | 3-1-1-6-6 | TCAaCtttcacTTCAGT | 17_17 | −26 |
| 17 | 2-1-2-6-6 | TCaACtttcacTTCAGT | 17_18 | −25 |
| 17 | 2-2-1-6-6 | TCaaCtttcacTTCAGT | 17_19 | −25 |
| 17 | 2-9-6 | TCaactttcacTTCAGT | 17_20 | −24 |
| 17 | 1-1-3-6-6 | TcAACtttcacTTCAGT | 17_21 | −24 |
| 17 | 1-1-2-1-1-5-6 | TcAAcTttcacTTCAGT | 17_22 | −23 |
| 17 | 1-3-1-6-6 | TcaaCtttcacTTCAGT | 17_23 | −23 |
| 17 | 5-6-3-1-2 | TCAACtttcacTTCaGT | 17_24 | −25 |
| 17 | 4-7-3-1-2 | TCAActttcacTTCaGT | 17_25 | −23 |
| 17 | 3-1-1-6-3-1-2 | TCAaCtttcacTTCaGT | 17_26 | −24 |
| 17 | 3-2-1-5-3-1-2 | TCAacTttcacTTCaGT | 17_27 | −23 |
| 17 | 3-8-3-1-2 | TCAactttcacTTCaGT | 17_28 | −23 |
| 17 | 2-1-2-6-3-1-2 | TCaACtttcacTTCaGT | 17_29 | −23 |
| 17 | 2-1-1-1-1-5-3-1-2 | TCaAcTttcacTTCaGT | 17_30 | −22 |
| 17 | 2-1-1-7-3-1-2 | TCaActttcacTTCaGT | 17_31 | −21 |
| 17 | 2-2-1-6-3-1-2 | TCaaCtttcacTTCaGT | 17_32 | −22 |
| 17 | 2-3-1-5-3-1-2 | TCaacTttcacTTCaGT | 17_33 | −22 |
| 17 | 2-9-3-1-2 | TCaactttcacTTCaGT | 17_34 | −21 |
| 17 | 1-1-3-6-3-1-2 | TcAACtttcacTTCaGT | 17_35 | −22 |
| 17 | 5-6-2-1-3 | TCAACtttcacTTcAGT | 17_36 | −24 |
| 17 | 4-1-1-5-2-1-3 | TCAAcTttcacTTcAGT | 17_37 | −23 |
| 17 | 2-1-1-1-1-5-2-1-3 | TCaAcTttcacTTcAGT | 17_38 | −22 |
| 17 | 1-1-2-1-1-5-2-1-3 | TcAAcTttcacTTcAGT | 17_39 | −21 |
| 17 | 1-2-1-1-1-5-2-1-3 | TcaAcTttcacTTcAGT | 17_40 | −20 |
| 17 | 1-3-1-6-2-1-3 | TcaaCtttcacTTcAGT | 17_41 | −21 |
| 17 | 1-4-1-5-2-1-3 | TcaacTttcacTTcAGT | 17_42 | −20 |
| 17 | 1-1-3-6-2-2-2 | TcAACtttcacTTcaGT | 17_43 | −21 |
| 17 | 1-1-1-1-1-6-2-2-2 | TcAaCtttcacTTcaGT | 17_44 | −20 |
| 17 | 1-3-1-6-2-2-2 | TcaaCtttcacTTcaGT | 17_45 | −19 |
| 17 | 5-6-1-1-4 | TCAACtttcacTtCAGT | 17_46 | −26 |
| 17 | 3-1-1-6-1-1-4 | TCAaCtttcacTtCAGT | 17_47 | −25 |
| 17 | 2-1-1-7-1-1-4 | TCaActttcacTtCAGT | 17_48 | −22 |
| 17 | 2-2-1-6-1-1-4 | TCaaCtttcacTtCAGT | 17_49 | −23 |
| 17 | 2-3-1-5-1-1-4 | TCaacTttcacTtCAGT | 17_50 | −23 |
| 17 | 2-9-1-1-4 | TCaactttcacTtCAGT | 17_51 | −22 |
| 17 | 1-3-1-6-1-1-4 | TcaaCtttcacTtCAGT | 17_52 | −22 |
| 17 | 5-6-1-1-1-1-2 | TCAACtttcacTtCaGT | 17_53 | −23 |
| 17 | 4-1-1-5-1-1-1-1-2 | TCAAcTttcacTtCaGT | 17_54 | −22 |
| 17 | 4-7-1-1-1-1-2 | TCAActttcacTtCaGT | 17_55 | −22 |
| 17 | 3-1-1-6-1-1-1-1-2 | TCAaCtttcacTtCaGT | 17_56 | −22 |
| 17 | 3-8-1-1-1-1-2 | TCAactttcacTtCaGT | 17_57 | −21 |
| 17 | 2-1-2-6-1-1-1-1-2 | TCaACtttcacTtCaGT | 17_58 | −21 |
| 17 | 2-1-1-1-1-5-1-1-1-1-2 | TCaAcTttcacTtCaGT | 17_59 | −20 |
| 17 | 2-1-1-7-1-1-1-1-2 | TCaActttcacTtCaGT | 17_60 | −20 |
| 17 | 2-2-2-5-1-1-1-1-2 | TCaaCTttcacTtCaGT | 17_61 | −22 |
| 17 | 2-2-1-6-1-1-1-1-2 | TCaaCtttcacTtCaGT | 17_62 | −21 |
| 17 | 2-3-1-5-1-1-1-1-2 | TCaacTttcacTtCaGT | 17_63 | −20 |
| 17 | 2-9-1-1-1-1-2 | TCaactttcacTtCaGT | 17_64 | −20 |
| 17 | 5-6-1-2-3 | TCAACtttcacTtcAGT | 17_65 | −23 |
| 17 | 4-1-1-5-1-2-3 | TCAAcTttcacTtcAGT | 17_66 | −23 |
| 17 | 4-7-1-2-3 | TCAActttcacTtcAGT | 17_67 | −22 |
| 17 | 3-1-1-6-1-2-3 | TCAaCtttcacTtcAGT | 17_68 | −22 |
| 17 | 3-2-1-5-1-2-3 | TCAacTttcacTtcAGT | 17_69 | −22 |
| 17 | 2-1-2-6-1-2-3 | TCaACtttcacTtcAGT | 17_70 | −22 |
| 17 | 2-1-1-1-1-5-1-2-3 | TCaAcTttcacTtcAGT | 17_71 | −21 |
| 17 | 1-1-2-1-1-5-1-2-3 | TcAAcTttcacTtcAGT | 17_72 | −20 |
| 17 | 5-6-1-3-2 | TCAACtttcacTtcaGT | 17_73 | −22 |
| 17 | 4-7-1-3-2 | TCAActttcacTtcaGT | 17_74 | −21 |
| 17 | 3-1-2-5-1-3-2 | TCAaCTttcacTtcaGT | 17_75 | −23 |
| 17 | 3-1-1-6-1-3-2 | TCAaCtttcacTtcaGT | 17_76 | −21 |
| 17 | 3-2-1-5-1-3-2 | TCAacTttcacTtcaGT | 17_77 | −21 |
| 17 | 2-1-2-6-1-3-2 | TCaACtttcacTtcaGT | 17_78 | −21 |
| 17 | 2-1-1-1-1-5-1-3-2 | TCaAcTttcacTtcaGT | 17_79 | −20 |
| 17 | 2-2-1-6-1-3-2 | TCaaCtttcacTtcaGT | 17_80 | −20 |
| 17 | 2-3-1-5-1-3-2 | TCaacTttcacTtcaGT | 17_81 | −19 |
| 17 | 1-1-3-6-1-3-2 | TcAACtttcacTtcaGT | 17_82 | −20 |
| 17 | 1-1-1-1-1-6-1-3-2 | TcAaCtttcacTtcaGT | 17_83 | −19 |
| 17 | 1-3-1-6-1-3-2 | TcaaCtttcacTtcaGT | 17_84 | −19 |
| 17 | 5-7-5 | TCAACtttcactTCAGT | 17_85 | −26 |
| 17 | 2-1-1-8-5 | TCaActttcactTCAGT | 17_86 | −23 |
| 17 | 2-2-1-7-5 | TCaaCtttcactTCAGT | 17_87 | −23 |
| 17 | 2-3-1-6-5 | TCaacTttcactTCAGT | 17_88 | −23 |
| 17 | 2-10-5 | TCaactttcactTCAGT | 17_89 | −23 |
| 17 | 1-1-2-8-5 | TcAActttcactTCAGT | 17_90 | −22 |
| 17 | 1-1-1-1-1-7-5 | TcAaCtttcactTCAGT | 17_91 | −22 |
| 17 | 1-3-1-7-5 | TcaaCtttcactTCAGT | 17_92 | −22 |
| 17 | 1-11-5 | TcaactttcactTCAGT | 17_93 | −21 |
| 17 | 5-7-2-1-2 | TCAACtttcactTCaGT | 17_94 | −24 |
| 17 | 4-1-1-6-2-1-2 | TCAAcTttcactTCaGT | 17_95 | −23 |
| 17 | 4-8-2-1-2 | TCAActttcactTCaGT | 17_96 | −22 |
| 17 | 3-1-1-7-2-1-2 | TCAaCtttcacTTCaGT | 17_97 | −22 |
| 17 | 3-2-1-6-2-1-2 | TCAacTttcactTCaGT | 17_98 | −22 |
| 17 | 3-9-2-1-2 | TCAactttcactTCaGT | 17_99 | −22 |
| 17 | 2-1-1-8-2-1-2 | TCaActttcactTCaGT | 17_100 | −20 |
| 17 | 2-10-2-1-2 | TCaactttcactTCaGT | 17_101 | −20 |
| 17 | 1-1-3-7-2-1-2 | TcAACtttcacTTCaGT | 17_102 | −21 |
| 17 | 1-1-2-8-2-1-2 | TcAActttcactTCaGT | 17_103 | −19 |
| 17 | 1-1-1-1-1-7-2-1-2 | TcAaCtttcacTTCaGT | 17_104 | −20 |
| 17 | 1-1-1-2-1-6-2-1-2 | TcAacTttcacTTCaGT | 17_105 | −19 |
| 17 | 1-1-1-9-2-1-2 | TcAactttcacTTCaGT | 17_106 | −19 |
| 17 | 1-3-1-7-2-1-2 | TcaaCtttcactTCaGT | 17_107 | −20 |
| 17 | 1-11-2-1-2 | TcaactttcacTTCaGT | 17_108 | −19 |
| 17 | 4-8-1-1-3 | TCAActttcacTTcAGT | 17_109 | −22 |
| 17 | 3-1-1-7-1-1-3 | TCAaCtttcacTTcAGT | 17_110 | −22 |
| 17 | 2-10-1-1-3 | TCaactttcacTTcAGT | 17_111 | −20 |
| 17 | 1-1-3-7-1-1-3 | TcAACtttcacTTcAGT | 17_112 | −21 |
| 17 | 1-1-2-8-1-1-3 | TcAActttcacTTcAGT | 17_113 | −19 |
| 17 | 1-1-1-1-1-7-1-1-3 | TcAaCtttcacTTcAGT | 17_114 | −20 |
| 17 | 1-2-1-8-1-1-3 | TcaActttcacTTcAGT | 17_115 | −19 |
| 17 | 1-3-1-7-1-1-3 | TcaaCtttcacTTcAGT | 17_116 | −20 |
| 17 | 1-11-1-1-3 | TcaactttcacTTcAGT | 17_117 | −19 |
| 17 | 5-7-1-2-2 | TCAACtttcacTTcaGT | 17_118 | −22 |
| 17 | 4-8-1-2-2 | TCAActttcacTTcaGT | 17_119 | −21 |
| 17 | 3-1-1-7-1-2-2 | TCAaCtttcacTTcaGT | 17_120 | −21 |
| 17 | 3-9-1-2-2 | TCAactttcacTTcaGT | 17_121 | −20 |
| 17 | 2-2-1-7-1-2-2 | TCaaCtttcacTTcaGT | 17_122 | −20 |
| 17 | 2-10-1-2-2 | TCaactttcacTTcaGT | 17_123 | −19 |
| 17 | 1-1-1-1-1-7-1-2-2 | TcAaCtttcacTTcaGT | 17_124 | −19 |
| 17 | 1-1-1-9-1-2-2 | TcAactttcacTTcaGT | 17_125 | −18 |
| 17 | 1-2-1-8-1-2-2 | TcaActttcacTTcaGT | 17_126 | −18 |
| 17 | 1-11-1-2-2 | TcaactttcacTTcaGT | 17_127 | −17 |
| 17 | 5-8-4 | TCAACtttcacttCAGT | 17_128 | −25 |
| 17 | 3-10-4 | TCAactttcacttCAGT | 17_129 | −23 |
| 17 | 2-1-2-8-4 | TCaACtttcacttCAGT | 17_130 | −23 |
| 17 | 2-1-1-1-1-7-4 | TCaAcTttcacttCAGT | 17_131 | −22 |
| 17 | 2-1-1-9-4 | TCaActttcacttCAGT | 17_132 | −22 |
| 17 | 2-2-1-8-4 | TCaaCtttcacttCAGT | 17_133 | −23 |
| 17 | 2-3-1-7-4 | TCaacTttcacttCAGT | 17_134 | −22 |
| 17 | 2-11-4 | TCaactttcacttCAGT | 17_135 | −22 |
| 17 | 1-1-3-8-4 | TcAACtttcacttCAGT | 17_136 | −22 |
| 17 | 1-1-2-9-4 | TcAActttcacttCAGT | 17_137 | −21 |
| 17 | 1-1-1-1-1-8-4 | TcAaCtttcacttCAGT | 17_138 | −21 |
| 17 | 1-1-1-10-4 | TcAactttcacttCAGT | 17_139 | −20 |
| 17 | 4-1-1-7-1-1-2 | TCAAcTttcacttCaGT | 17_140 | −22 |
| 17 | 3-1-2-7-1-1-2 | TCAaCTttcacttCaGT | 17_141 | −23 |
| 17 | 3-1-1-8-1-1-2 | TCAaCtttcacttCaGT | 17_142 | −22 |
| 17 | 3-2-1-7-1-1-2 | TCAacTttcacttCaGT | 17_143 | −21 |
| 17 | 3-10-1-1-2 | TCAactttcacttCaGT | 17_144 | −21 |
| 17 | 2-1-3-7-1-1-2 | TCaACTttcacttCaGT | 17_145 | −22 |
| 17 | 2-1-2-8-1-1-2 | TCaACtttcacttCaGT | 17_146 | −21 |
| 17 | 2-1-1-1-1-7-1-1-2 | TCaAcTttcacttCaGT | 17_147 | −20 |
| 17 | 2-2-2-7-1-1-2 | TCaaCTttcacttCaGT | 17_148 | −21 |
| 17 | 2-3-1-7-1-1-2 | TCaacTttcacttCaGT | 17_149 | −20 |
| 17 | 1-1-3-8-1-1-2 | TcAACtttcacttCaGT | 17_150 | −20 |
| 17 | 1-1-1-1-1-8-1-1-2 | TcAaCtttcacttCaGT | 17_151 | −19 |
| 17 | 1-1-1-10-1-1-2 | TcAactttcacttCaGT | 17_152 | −18 |
| 17 | 1-2-1-9-1-1-2 | TcaActttcacttCaGT | 17_153 | −18 |
| 17 | 1-3-2-7-1-1-2 | TcaaCTttcacttCaGT | 17_154 | −20 |
| 17 | 1-12-1-1-2 | TcaactttcacttCaGT | 17_155 | −18 |
| 17 | 4-1-1-8-3 | TCAAcTttcacttcAGT | 17_156 | −22 |
| 17 | 4-10-3 | TCAActttcacttcAGT | 17_157 | −22 |
| 17 | 3-1-2-8-3 | TCAaCTttcacttcAGT | 17_158 | −23 |
| 17 | 3-1-1-9-3 | TCAaCtttcacttcAGT | 17_159 | −22 |
| 17 | 2-2-2-8-3 | TCaaCTttcacttcAGT | 17_160 | −22 |
| 17 | 2-3-1-8-3 | TCaacTttcacttcAGT | 17_161 | −20 |
| 17 | 1-1-1-1-1-9-3 | TcAaCtttcacttcAGT | 17_162 | −19 |
| 17 | 1-1-1-11-3 | TcAactttcacttcAGT | 17_163 | −18 |
| 17 | 1-2-1-10-3 | TcaActttcacttcAGT | 17_164 | −19 |
| 17 | 1-13-3 | TcaactttcacttcAGT | 17_165 | −18 |
| 17 | 6-9-2 | TCAACTttcacttcaGT | 17_166 | −23 |
| 17 | 5-10-2 | TCAACtttcacttcaGT | 17_167 | −22 |
| 17 | 4-1-1-9-2 | TCAAcTttcacttcaGT | 17_168 | −21 |
| 17 | 4-11-2 | TCAActttcacttcaGT | 17_169 | −20 |
| 17 | 3-1-2-9-2 | TCAaCTttcacttcaGT | 17_170 | −22 |
| 17 | 3-1-1-10-2 | TCAaCtttcacttcaGT | 17_171 | −21 |
| 17 | 3-12-2 | TCAactttcacttcaGT | 17_172 | −20 |
| 17 | 2-1-3-9-2 | TCaACTttcacttcaGT | 17_173 | −21 |
| 17 | 2-1-2-10-2 | TCaACtttcacttcaGT | 17_174 | −20 |
| 17 | 2-1-1-11-2 | TCaActttcacttcaGT | 17_175 | −19 |
| 17 | 2-2-1-10-2 | TCaaCtttcacttcaGT | 17_176 | −19 |
| 17 | 2-3-1-9-2 | TCaacTttcacttcaGT | 17_177 | −19 |
| 17 | 1-1-2-11-2 | TcAActttcacttcaGT | 17_178 | −18 |
| 17 | 1-1-1-1-1-10-2 | TcAaCtttcacttcaGT | 17_179 | −18 |
| 17 | 1-1-1-12-2 | TcAactttcacttcaGT | 17_180 | −17 |
| 17 | 1-2-1-11-2 | TcaActttcacttcaGT | 17_181 | −17 |
| 17 | 1-3-1-10-2 | TcaaCtttcacttcaGT | 17_182 | −18 |
| 17 | 1-14-2 | TcaactttcacttcaGT | 17_183 | −17 |
| 18 | 3-10-3 | TCAactttcacttCAG | 18_1 | −19 |
| 18 | 2-2-1-6-5 | TCaaCtttcacTTCAG | 18_2 | −21 |
| 18 | 1-1-3-6-2-1-2 | TcAACtttcacTTcAG | 18_3 | −18 |
| 18 | 5-6-1-1-3 | TCAACtttcacTtCAG | 18_4 | −22 |
| 18 | 4-7-1-1-3 | TCAActttcacTtCAG | 18_5 | −20 |
| 18 | 2-9-1-1-3 | TCaactttcacTtCAG | 18_6 | −18 |
| 18 | 1-3-1-6-1-1-3 | TcaaCtttcacTtCAG | 18_7 | −18 |
| 18 | 2-1-1-7-1-2-2 | TCaActttcacTtcAG | 18_8 | −17 |
| 18 | 5-7-4 | TCAACtttcactTCAG | 18_9 | −22 |
| 18 | 4-8-4 | TCAActttcactTCAG | 18_10 | −21 |
| 18 | 3-1-1-7-4 | TCAaCtttcactTCAG | 18_11 | −21 |
| 18 | 3-9-4 | TCAactttcactTCAG | 18_12 | −20 |
| 18 | 2-2-1-7-4 | TCaaCtttcactTCAG | 18_13 | −20 |
| 18 | 2-10-4 | TCaactttcactTCAG | 18_14 | −19 |
| 18 | 1-1-3-7-1-1-2 | TcAACtttcacTTcAG | 18_15 | −17 |
| 18 | 1-1-1-1-1-7-1-1-2 | TcAaCtttcacTTcAG | 18_16 | −16 |
| 18 | 1-3-1-7-1-1-2 | TcaaCtttcacTTcAG | 18_17 | −16 |
| 18 | 5-8-3 | TCAACtttcacttCAG | 18_18 | −21 |
| 18 | 4-9-3 | TCAActttcacttCAG | 18_19 | −20 |
| 18 | 3-1-1-8-3 | TCAaCtttcacttCAG | 18_20 | −20 |
| 18 | 2-2-1-8-3 | TCaaCtttcacttCAG | 18_21 | −19 |
| 18 | 2-11-3 | TCaactttcacttCAG | 18_22 | −18 |
| 18 | 5-9-2 | TCAACtttcacttcAG | 18_23 | −19 |
| 18 | 4-10-2 | TCAActttcacttcAG | 18_24 | −18 |
| 18 | 3-1-1-9-2 | TCAaCtttcacttcAG | 18_25 | −18 |
| 18 | 3-11-2 | TCAactttcacttcAG | 18_26 | −17 |
| 18 | 2-1-2-9-2 | TCaACtttcacttcAG | 18_27 | −17 |
| 18 | 2-2-1-9-2 | TCaaCtttcacttcAG | 18_28 | −17 |
| 18 | 2-12-2 | TCaactttcacttcAG | 18_29 | −16 |
| 18 | 1-1-3-9-2 | TcAACtttcacttcAG | 18_30 | −16 |
| 18 | 1-3-1-9-2 | TcaaCtttcacttcAG | 18_31 | −15 |
| 18 | 3-10-3 | TCAactttcacttCAG | 18_249 | −19 |
| 18 | 5-5-6 | TCAACtttcaCTTCAG | 18_250 | −25 |
| 18 | 4-6-6 | TCAActttcaCTTCAG | 18_251 | −24 |
| 18 | 3-1-1-5-6 | TCAaCtttcaCTTCAG | 18_252 | −24 |
| 18 | 2-1-2-5-6 | TCaACtttcaCTTCAG | 18_253 | −23 |
| 18 | 2-2-1-5-6 | TCaaCtttcaCTTCAG | 18_254 | −22 |
| 18 | 1-3-1-5-6 | TcaaCtttcaCTTCAG | 18_255 | −21 |
| 18 | 1-9-6 | TcaactttcaCTTCAG | 18_256 | −20 |
| 18 | 1-1-1-1-1-5-3-1-2 | TcAaCtttcaCTTcAG | 18_257 | −19 |
| 18 | 1-3-1-5-3-1-2 | TcaaCtttcaCTTcAG | 18_258 | −18 |
| 18 | 1-9-3-1-2 | TcaactttcaCTTcAG | 18_259 | −17 |
| 18 | 3-1-1-5-2-1-3 | TCAaCtttcaCTtCAG | 18_260 | −22 |
| 18 | 3-7-2-1-3 | TCAactttcaCTtCAG | 18_261 | −21 |
| 18 | 2-2-1-5-2-1-3 | TCaaCtttcaCTtCAG | 18_262 | −21 |
| 18 | 2-8-2-1-3 | TCaactttcaCTtCAG | 18_263 | −20 |
| 18 | 1-1-3-5-2-1-3 | TcAACtttcaCTtCAG | 18_264 | −21 |
| 18 | 1-3-1-5-2-1-3 | TcaaCtttcaCTtCAG | 18_265 | −20 |
| 18 | 1-9-2-1-3 | TcaactttcaCTtCAG | 18_266 | −19 |
| 18 | 5-5-2-2-2 | TCAACtttcaCTtcAG | 18_267 | −21 |
| 18 | 4-6-2-2-2 | TCAActttcaCTtcAG | 18_268 | −20 |
| 18 | 3-1-1-5-2-2-2 | TCAaCtttcaCTtcAG | 18_269 | −20 |
| 18 | 3-7-2-2-2 | TCAactttcaCTtcAG | 18_270 | −19 |
| 18 | 2-1-2-5-2-2-2 | TCaACtttcaCTtcAG | 18_271 | −20 |
| 18 | 2-1-1-6-2-2-2 | TCaActttcaCTtcAG | 18_272 | −18 |
| 18 | 1-1-1-1-1-5-2-2-2 | TcAaCtttcaCTtcAG | 18_273 | −18 |
| 18 | 1-3-1-5-2-2-2 | TcaaCtttcaCTtcAG | 18_274 | −18 |
| 18 | 5-5-1-1-4 | TCAACtttcaCtTCAG | 18_275 | −23 |
| 18 | 4-6-1-1-4 | TCAActttcaCtTCAG | 18_276 | −22 |
| 18 | 3-1-1-5-1-1-4 | TCAaCtttcaCtTCAG | 18_277 | −22 |
| 18 | 3-7-1-1-4 | TCAactttcaCtTCAG | 18_278 | −21 |
| 18 | 2-1-2-5-1-1-4 | TCaACtttcaCtTCAG | 18_279 | −22 |
| 18 | 2-1-1-6-1-1-4 | TCaActttcaCtTCAG | 18_280 | −20 |
| 18 | 2-2-1-5-1-1-4 | TCaaCtttcaCtTCAG | 18_281 | −21 |
| 18 | 2-8-1-1-4 | TCaactttcaCtTCAG | 18_282 | −20 |
| 18 | 2-2-1-5-1-1-1-1-2 | TCaaCtttcaCtTcAG | 18_283 | −18 |
| 18 | 2-8-1-1-1-1-2 | TCaactttcaCtTcAG | 18_284 | −17 |
| 18 | 1-1-3-5-1-1-1-1-2 | TcAACtttcaCtTcAG | 18_285 | −18 |
| 18 | 1-1-2-6-1-1-1-1-2 | TcAActttcaCtTcAG | 18_286 | −16 |
| 18 | 1-1-1-1-1-5-1-1-1-1-2 | TcAaCtttcaCtTcAG | 18_287 | −17 |
| 18 | 1-1-1-7-1-1-1-1-2 | TcAactttcaCtTcAG | 18_288 | −16 |
| 18 | 1-2-1-6-1-1-1-1-2 | TcaActttcaCtTcAG | 18_289 | −16 |
| 18 | 1-3-1-5-1-1-1-1-2 | TcaaCtttcaCTTcAG | 18_290 | −17 |
| 18 | 1-9-1-1-1-1-2 | TcaactttcaCtTcAG | 18_291 | −16 |
| 18 | 5-5-1-2-3 | TCAACtttcaCttCAG | 18_292 | −22 |
| 18 | 4-6-1-2-3 | TCAActttcaCttCAG | 18_293 | −21 |
| 18 | 3-1-1-5-1-2-3 | TCAaCtttcaCttCAG | 18_294 | −21 |
| 18 | 3-7-1-2-3 | TCAactttcaCttCAG | 18_295 | −20 |
| 18 | 2-1-2-5-1-2-3 | TCaACtttcaCttCAG | 18_296 | −21 |
| 18 | 2-1-1-6-1-2-3 | TCaActttcaCttCAG | 18_297 | −19 |
| 18 | 2-2-1-5-1-2-3 | TCaaCtttcaCttCAG | 18_298 | −20 |
| 18 | 2-8-1-2-3 | TCaactttcaCttCAG | 18_299 | −19 |
| 18 | 1-1-3-5-1-2-3 | TcAACtttcaCttCAG | 18_300 | −20 |
| 18 | 1-2-2-5-1-2-3 | TcaACtttcaCttCAG | 18_301 | −19 |
| 18 | 1-2-1-6-1-2-3 | TcaActttcaCttCAG | 18_302 | −18 |
| 18 | 5-5-1-3-2 | TCAACtttcaCttcAG | 18_303 | −20 |
| 18 | 4-6-1-3-2 | TCAActttcaCttcAG | 18_304 | −19 |
| 18 | 3-1-1-5-1-3-2 | TCAaCtttcaCttcAG | 18_305 | −19 |
| 18 | 3-7-1-3-2 | TCAactttcaCttcAG | 18_306 | −18 |
| 18 | 2-1-2-5-1-3-2 | TCaACtttcaCttcAG | 18_307 | −18 |
| 18 | 2-1-1-6-1-3-2 | TCaActttcaCttcAG | 18_308 | −17 |
| 18 | 2-2-1-5-1-3-2 | TCaaCtttcaCttcAG | 18_309 | −18 |
| 18 | 2-8-1-3-2 | TCaactttcaCttcAG | 18_310 | −17 |
| 18 | 1-1-3-5-1-3-2 | TcAACtttcaCttcAG | 18_311 | −17 |
| 18 | 1-1-2-6-1-3-2 | TcAActttcaCttcAG | 18_312 | −16 |
| 18 | 1-1-1-1-1-5-1-3-2 | TcAaCtttcaCttcAG | 18_313 | −16 |
| 18 | 1-1-1-7-1-3-2 | TcAactttcaCttcAG | 18_314 | −15 |
| 18 | 1-2-2-5-1-3-2 | TcaACtttcaCttcAG | 18_315 | −17 |
| 18 | 1-3-1-5-1-3-2 | TcaaCtttcaCttcAG | 18_316 | −16 |
| 18 | 1-9-1-3-2 | TcaactttcaCttcAG | 18_317 | −15 |
| 18 | 4-7-5 | TCAActttcacTTCAG | 18_318 | −22 |
| 18 | 3-1-1-6-5 | TCAaCtttcacTTCAG | 18_319 | −22 |
| 18 | 2-1-2-6-5 | TCaACtttcacTTCAG | 18_320 | −22 |
| 18 | 1-1-3-6-5 | TcAACtttcacTTCAG | 18_321 | −21 |
| 18 | 1-1-1-1-1-6-5 | TcAaCtttcacTTCAG | 18_322 | −20 |
| 18 | 1-3-1-6-5 | TcaaCtttcacTTCAG | 18_323 | −19 |
| 18 | 5-6-2-1-2 | TCAACtttcacTTcAG | 18_324 | −21 |
| 18 | 3-1-1-6-2-1-2 | TCAaCtttcacTTcAG | 18_325 | −20 |
| 18 | 2-2-1-6-2-1-2 | TCaaCtttcacTTcAG | 18_326 | −18 |
| 18 | 1-1-2-7-2-1-2 | TcAActttcacTTcAG | 18_327 | −16 |
| 18 | 1-1-1-1-1-6-2-1-2 | TcAaCtttcacTTcAG | 18_328 | −17 |
| 18 | 1-1-1-8-2-1-2 | TcAactttcacTTcAG | 18_329 | −16 |
| 18 | 1-3-1-6-2-1-2 | TcaaCtttcacTTcAG | 18_330 | −17 |
| 18 | 1-10-2-1-2 | TcaactttcacTTcAG | 18_331 | −16 |
| 18 | 3-1-1-6-1-1-3 | TCAaCtttcacTtCAG | 18_332 | −21 |
| 18 | 2-1-1-7-1-1-3 | TCaActttcacTtCAG | 18_333 | −19 |
| 18 | 2-2-1-6-1-1-3 | TCaaCtttcacTtCAG | 18_334 | −19 |
| 18 | 1-1-2-7-1-1-3 | TcAActttcacTtCAG | 18_335 | −18 |
| 18 | 1-10-1-1-3 | TcaactttcacTtCAG | 18_336 | −17 |
| 18 | 5-6-1-2-2 | TCAACtttcacTtcAG | 18_337 | −20 |
| 18 | 4-7-1-2-2 | TCAActttcacTtcAG | 18_338 | −18 |
| 18 | 3-1-1-6-1-2-2 | TCAaCtttcacTtcAG | 18_339 | −19 |
| 18 | 2-2-1-6-1-2-2 | TCaaCtttcacTtcAG | 18_340 | −17 |
| 18 | 2-9-1-2-2 | TCaactttcacTtcAG | 18_341 | −16 |
| 18 | 1-1-3-6-1-2-2 | TcAACtttcacTtcAG | 18_342 | −17 |
| 18 | 1-1-1-1-1-6-1-2-2 | TcAaCtttcacTtcAG | 18_343 | −16 |
| 18 | 1-3-1-6-1-2-2 | TcaaCtttcacTtcAG | 18_344 | −16 |
| 18 | 2-1-2-7-4 | TCaACtttcactTCAG | 18_345 | −21 |
| 18 | 2-1-1-8-4 | TCaActttcactTCAG | 18_346 | −19 |
| 18 | 1-1-2-8-4 | TcAActttcactTCAG | 18_347 | −18 |
| 18 | 1-2-1-8-4 | TcaActttcactTCAG | 18_348 | −18 |
| 18 | 1-11-4 | TcaactttcactTCAG | 18_349 | −17 |
| 18 | 4-8-1-1-2 | TCAActttcacTTcAG | 18_350 | −18 |
| 18 | 2-2-1-7-1-1-2 | TCaaCtttcacTTcAG | 18_351 | −17 |
| 18 | 2-10-1-1-2 | TCaactttcacTTcAG | 18_352 | −16 |
| 18 | 1-1-2-8-1-1-2 | TcAActttcacTTcAG | 18_353 | −15 |
| 18 | 1-2-2-7-1-1-2 | TcaACtttcacTTcAG | 18_354 | −17 |
| 18 | 1-2-1-8-1-1-2 | TcaActttcacTTcAG | 18_355 | −15 |
| 18 | 2-1-2-8-3 | TCaACtttcacttCAG | 18_356 | −20 |
| 18 | 2-1-1-9-3 | TCaActttcacttCAG | 18_357 | −18 |
| 18 | 1-2-2-8-3 | TcaACtttcacttCAG | 18_358 | −18 |
| 18 | 1-2-1-9-3 | TcaActttcacttCAG | 18_359 | −17 |
| 18 | 1-12-3 | TcaactttcacttCAG | 18_360 | −16 |
| 18 | 1-1-1-1-1-9-2 | TcAaCtttcacttcAG | 18_361 | −15 |
| 19 | 5-6-5 | TGTTTcaatacTAAAA | 19_1 | −16 |
| 19 | 4-7-5 | TGTTtcaatacTAAAA | 19_2 | −15 |
| 19 | 5-6-2-1-2 | TGTTTcaatacTAaAA | 19_3 | −16 |
| 19 | 5-5-6 | TGTTTcaataCTAAAA | 19_4 | −18 |
| 19 | 4-6-6 | TGTTtcaataCTAAAA | 19_5 | −17 |
| 19 | 3-1-1-5-6 | TGTtTcaataCTAAAA | 19_6 | −17 |
| 19 | 3-7-6 | TGTttcaataCTAAAA | 19_7 | −16 |
| 19 | 2-1-2-5-6 | TGtTTcaataCTAAAA | 19_8 | −16 |
| 19 | 2-2-1-5-6 | TGttTcaataCTAAAA | 19_9 | −15 |
| 19 | 1-1-3-5-6 | TgTTTcaataCTAAAA | 19_10 | −16 |
| 19 | 5-5-3-1-2 | TGTTTcaataCTAaAA | 19_11 | −17 |
| 19 | 4-6-3-1-2 | TGTTtcaataCTAaAA | 19_12 | −16 |
| 19 | 3-1-1-5-3-1-2 | TGTtTcaataCTAaAA | 19_13 | −16 |
| 19 | 3-7-3-1-2 | TGTttcaataCTAaAA | 19_14 | −16 |
| 19 | 2-1-2-5-3-1-2 | TGtTTcaataCTAaAA | 19_15 | −15 |
| 19 | 1-1-3-5-3-1-2 | TgTTTcaataCTAaAA | 19_16 | −15 |
| 19 | 5-5-2-1-3 | TGTTTcaataCTaAAA | 19_17 | −17 |
| 19 | 4-6-2-1-3 | TGTTtcaataCTaAAA | 19_18 | −16 |
| 19 | 3-1-1-5-2-1-3 | TGTtTcaataCTaAAA | 19_19 | −15 |
| 19 | 5-5-2-2-2 | TGTTTcaataCTaaAA | 19_20 | −16 |
| 19 | 4-6-2-2-2 | TGTTtcaataCTaaAA | 19_21 | −15 |
| 19 | 5-5-1-1-4 | TGTTTcaataCtAAAA | 19_22 | −15 |

Designs refer to the gapmer design, F-G-F′. In classic gapmer design e.g. 3-1 0-3 all the nucleotides in the flanks (F and F′) are constituted of the same 2′-sugar modified nucleoside, e.g. LNA, cET, or MOE, and a stretch of DNA in the middle forming the gap (G). In gapmers with alternating flank designs the flanks of oligonucleotide is annotated as a series of integers, representing a number of 2′ sugar modified nucleosides (M) followed by a number of DNA nucleosides (D). For example a flank with a 2-2-1 motif represents 5′ [M]₂-[D]₂-[M]3′ and a 1-1-1-1-1 motif represents 5′ [M]-[D]-[M]-[D]-[M]3′. Both flanks have a 2′ sugar modified nucleoside at the 5′ and 3′ terminal. The gap region (G), which is constituted of a number of DNA nucleosides (typically between 5 and 16), is located between the flanks.

The heading “Oligonucleotide compound” in the table represents specific designs of a motif sequence. Capital letters represent beta-D-oxy LNA nucleosides, lowercase letters represent DNA nucleosides, all LNA C are 5-methyl cytosine, and 5-methyl cytosine DNA are presented by “e”, all internucleoside linkages are phosphorothioate internucleoside linkages.

TABLE 8
List of stereodefined variants.
The parent oligonucleotide compound is indicated with its sequence
motif and design. The stereodefinition motif of the internucleoside
linkages of the parent compound is indicated below the sequence
and design, and reflects a fully stereorandom phosphorthioate
gapmer. The stereodefined variants of the parent are listed by
CMP ID NO and stereodefined motifs below the parent compound.
The table contain three parent compounds CMP ID NO: 18_1, 18_347
and 18_12.

| | | | Parent Compound/ |
|---|---|---|---|
| SEQ ID NO | Design | CMP ID NO | stereodefinition |
| 18 | 3-10-3 | 18_1 | TCAactttcacttCAG |
| | | | XXXXXXXXXXXXXXXH |
| CMP ID NO | Stereodefined motif | CMP ID NO | Stereodefined motif |
| 18_32 | RSSRXXXXXXXXXXXH | 18_365 | SSSSSRSRRXXXXXXH |
| 18_33 | XRSSRXXXXXXXXXXH | 18_366 | SSSSSSRRRXXXXXXH |
| 18_34 | XXRSSRXXXXXXXXXH | 18_367 | SSSRSRRRRXXXXXXH |
| 18_35 | XXXRSSRXXXXXXXXH | 18_368 | SSRSSRRRRXXXXXXH |
| 18_36 | XXXXRSSRXXXXXXXH | 18_369 | SSRRSSRRRXXXXXXH |
| 18_37 | XXXXXRSSRXXXXXXH | 18_370 | SSRRSRSRRXXXXXXH |
| 18_38 | XXXXXXRSSRXXXXXH | 18_371 | SSRRSSSRRXXXXXXH |
| 18_39 | XXXXXXXRSSRXXXXH | 18_372 | SSSRSRSRRXXXXXXH |
| 18_40 | XXXXXXXXRSSRXXXH | 18_373 | SSSSSRRRRXXXXXXH |
| 18_41 | XXXXXXXXXRSSRXXH | 18_374 | SSRSSSRRRXXXXXXH |
| 18_42 | XXXXXXXXXXRSSRXH | 18_375 | SSRSSRSRRXXXXXXH |
| 18_43 | XXXXXXXXXXXRSSRH | 18_376 | SSSRSSRRRXXXXXXH |
| 18_44 | XXXXXXXXXSSSSSRH | 18_377 | SSRRSRRRRXXXXXXH |
| 18_45 | XXXXXXXXXRRRRRRH | 18_378 | RSSRRSSSSRRRRSSH |
| 18_46 | XXXXXXXXXSSRRSRH | 18_379 | SRSRRSSSSRRRRSSH |
| 18_47 | XXXXXXXXXSSSRSRH | 18_380 | SSRRRSSSSRRRRSSH |
| 18_48 | XXXXXXXXXSSSRRSH | 18_381 | SSSSRSSSSRRRRSSH |
| 18_49 | XXXXXXXXXSRSSSSH | 18_382 | SSSRSSSSSRRRRSSH |
| 18_50 | XXXXXXXXXRSRSRSH | 18_383 | SSSRRRSSSRRRRSSH |
| 18_51 | XXXXXXXXXSSSSRSH | 18_384 | SSSRRSRSSRRRRSSH |
| 18_52 | XXXXXXXXXSSRRSSH | 18_385 | SSSRRSSRSRRRRSSH |
| 18_53 | XXXXXXXXXRRSSSSH | 18_386 | SSSRRSSSRRRRRSSH |
| 18_54 | XXXXXXXXXRSSRRRH | 18_387 | SSSRRSSSSSRRRSSH |
| 18_55 | XXXXXXXXXSRRRRSH | 18_388 | SSSRRSSSSSSRRSSH |
| 18_56 | XXXXXXXXXSSRSRRH | 18_389 | SSSRRSSSSRRSRSSH |
| 18_57 | XXXXXXXXXRRRSRRH | 18_390 | SSSRRSSSSRRRSSSH |
| 18_58 | XXXXXXXXXRRSRSRH | 18_391 | SSSRRSSSSRRRRRSH |
| 18_59 | XXXXXXXXXSSRRRSH | 18_392 | SSSRRSSSSRRRRSRH |
| 18_60 | XXXXXXXXXSRRSSSH | 18_393 | SRSSRSSSSRRRRSSH |
| 18_61 | XXXXXXXXXRRRRRSH | 18_394 | SSRSSRSSSRRRRSSH |
| 18_62 | XXXXXXXXXRRSSRRH | 18_395 | SSSRSSRSSRRRRSSH |
| 18_63 | XXXXXXXXXRSRRRRH | 18_396 | SSSRRRSSRRRRRSSH |
| 18_64 | XXXXXXXXXSRRRSSH | 18_397 | SSSRRSSRSSRRRSSH |
| 18_65 | XXXXXXXXXSRSRSRH | 18_398 | SSSRRSSSRSSRRSSH |
| 18_66 | XXXXXXXXXRSSSSRH | 18_399 | SSSRRSSSSRSSRSSH |
| 18_67 | XXXXXXXXXSSSSRRH | 18_400 | SSSRRSSSSRRSSRSH |
| 18_68 | XXXXXXXXXRRSSSRH | 18_401 | SSSRRSSSSRRRSSRH |
| 18_69 | XXXXXXXXXRSSRRSH | 18_402 | RSSRRSSSSRRRSSRH |
| 18_70 | XXXXXXXXXRSSSRRH | 18_403 | SRSSRSSSSRRSSRSH |
| 18_71 | XXXXXXXXXSRRRRRH | 18_404 | SSRSSRSSSRSSRSSH |
| 18_72 | XXXXXXXXXRRSRSSH | 18_405 | SSSRSSRSRSSRRSSH |
| 18_73 | XXXXXXXXXRSRSSRH | 18_406 | SSSRRSSRRSSRRSSH |
| 18_74 | XXXXXXXXXRSRSRRH | 18_407 | RSSRRRSSRRRRSSRH |
| 18_75 | XXXXXXXXXSRRRSRH | 18_408 | SSSRSSSRRRRRXXXH |
| 18_76 | XXXXXXXXXRRSRRSH | 18_409 | SSSSSSSRRRRRXXXH |
| 18_77 | XXXXXXXXXSSSRRRH | 18_410 | SSSRSSSRRSRRXXXH |
| 18_78 | XXXXXXXXXRSRRSRH | 18_411 | SSSRSSSRRRSRXXXH |
| 18_79 | XXXXXXXXXSRRSRSH | 18_412 | SSSSSSSRRSSRXXXH |
| 18_80 | XXXXXXXXXRRSRRRH | 18_413 | SSSSSSSRRSRRXXXH |
| 18_81 | XXXXXXXXXSRRSSRH | 18_414 | SSSSSSSRRRSRXXXH |
| 18_82 | XXXXXXXXXSRSSSRH | 18_415 | SSSRSSSRRSSRXXXH |
| 18_83 | XXXXXXXXXRSRRRSH | 18_416 | SSRRSRRRRXXRXXXH |
| 18_84 | XXXXXXXXXSSSRSSH | 18_417 | SSSRSRRRRXXRXXXH |
| 18_85 | XXXXXXXXXSSRSSRH | 18_418 | SSRSSRRRRXXRXXXH |
| 18_86 | XXXXXXXXXRSSRSSH | 18_419 | SSRRSSRRRXXRXXXH |
| 18_87 | XXXXXXXXXSRSSRSH | 18_420 | SSRRSRSRRXXRXXXH |
| 18_88 | XXXXXXXXXSSSSSSH | 18_421 | SSSSSSSRRXXRXXXH |
| 18_89 | XXXXXXXXXRSRRSSH | 18_422 | SSRSSSSRRXXRXXXH |
| 18_90 | XXXXXXXXXRRRRSRH | 18_423 | SSSRSSSRRXXRXXXH |
| 18_91 | XXXXXXXXXSSRSRSH | 18_424 | SSSSSSRRRXXRXXXH |
| 18_92 | XXXXXXXXXRRRRSSH | 18_425 | SSSSSRSRRXXRXXXH |
| 18_93 | XXXXXXXXXRSRSSSH | 18_426 | SSRSSRSRRXXRXXXH |
| 18_94 | XXXXXXXXXRSSRSRH | 18_427 | SSSRSRSRRXXRXXXH |
| 18_95 | XXXXXXXXXRRRSRSH | 18_428 | SSSRSSRRRXXRXXXH |
| 18_96 | XXXXXXXXXRRSSRSH | 18_429 | SSRSSSRRRXXRXXXH |
| 18_97 | XXXXXXXXXSRSSRRH | 18_430 | SSRRSSSRRXXRXXXH |
| 18_98 | XXXXXXXXXSRRSRRH | 18_431 | SSSSSRRRRXXRXXXH |
| 18_99 | XXXXXXXXXSRSRSSH | 18_432 | SSSRRSSSSRSRRSSH |
| 18_100 | XXXXXXXXXSRSRRRH | 18_433 | XXXXRSSRXSSSRXXH |
| 18_101 | XXXXXXXXXSSRSSSH | 18_434 | XXXXRSSRXSSRRXXH |
| 18_102 | XXXXXXXXXRSSSSSH | 18_435 | XXXXRSSRXRSSRXXH |
| 18_103 | XXXXXXXXXRSSSRSH | 18_436 | XXXXRSSRXSRSSXXH |
| 18_104 | XXXXXXXXXRRRSSRH | 18_437 | XXXXRSSRXRRRRXXH |
| 18_105 | XXXXXXXXXRRRSSSH | 18_438 | XXXXRSSRXRRSRXXH |
| 18_106 | XXXXXXXXXSRSRRSH | 18_439 | XXXXRSSRXSRRRXXH |
| 18_107 | XXXXXXXXXSSRRRRH | 18_440 | XXXXRSSRXRRSSXXH |
| 18_108 | XXXXXXXXXXSSRSSH | 18_441 | XXXXRSSRXRSRRXXH |
| 18_109 | XXXXXXXXXXRRRSSH | 18_442 | XXXXRSSRXRSSSXXH |
| 18_110 | XXXXXXXXXXRRSSRH | 18_443 | XXXXRSSRXRRRSXXH |
| 18_111 | XXXXXXXXXXRSSSRH | 18_444 | XXXXRSSRXRSRSXXH |
| 18_112 | XXXXXXXXXXRRSRRH | 18_445 | XXXXRSSRXSRRSXXH |
| 18_113 | XXXXXXXXXXSSSSRH | 18_446 | XXXXRSSRXSSSSXXH |
| 18_114 | XXXXXXXXXXRRRRRH | 18_447 | XXXXRSSRXSRSRXXH |
| 18_115 | XXXXXXXXXXSRSSSH | 18_448 | XXXXRSSRXSSRSXXH |
| 18_116 | XXXXXXXXXXSSRSRH | 18_449 | SSSRRSSSRRSSRSSH |
| 18_117 | XXXXXXXXXXRSSRSH | 18_450 | RSSRRSSSRRRRRSSH |
| 18_118 | XXXXXXXXXXRSRRRH | 18_451 | SRSRRSSSRRRRRSSH |
| 18_119 | XXXXXXXXXXSRRRRH | 18_452 | SSRRRSSSRRRRRSSH |
| 18_120 | XXXXXXXXXXSRRRSH | 18_453 | SSSSRSSSRRRRRSSH |
| 18_121 | XXXXXXXXXXSSSRSH | 18_454 | SSSRSSSSRRRRRSSH |
| 18_122 | XXXXXXXXXXRSRSSH | 18_455 | SSSRRSRSRRRRRSSH |
| 18_123 | XXXXXXXXXXSSSSSH | 18_456 | SSSRRSSRRRRRRSSH |
| 18_124 | XXXXXXXXXXSRRSSH | 18_457 | SSSRRSSSRSRRRSSH |
| 18_125 | XXXXXXXXXXRSRRSH | 18_458 | SSSRRSSSRRSRRSSH |
| 18_126 | XXXXXXXXXXSSRRSH | 18_459 | SSSRRSSSRRRSRSSH |
| 18_127 | XXXXXXXXXXRRRSRH | 18_460 | SSSRRSSSRRRRSSSH |
| 18_128 | XXXXXXXXXXSRSRRH | 18_461 | SSSRRSSSRRRRRRSH |
| 18_129 | XXXXXXXXXXRRSRSH | 18_462 | SSSRRSSSRRRRRSRH |
| 18_130 | XXXXXXXXXXRRSSSH | 18_463 | SSSRRSSSRRRSSSRH |
| 18_131 | XXXXXXXXXXRSSSSH | 18_464 | SSSRRSSSRRRSRRSH |
| 18_132 | XXXXXXXXXXRSSRRH | 18_465 | XXXXRSSRXRRSRRSH |
| 18_133 | XXXXXXXXXXSRRSRH | 18_466 | XXXXRSSRXXRSSSRH |
| 18_134 | XXXXXXXXXXSSRRRH | 18_467 | SSXXSXXRRXXRXXXH |
| 18_135 | XXXXXXXXXXSRSSRH | 18_468 | SSXXSXXRRXXXXXXH |
| 18_136 | XXXXXXXXXXRRRRSH | 18_469 | SSSXSSSRRXXRXXXH |
| 18_137 | XXXXXXXXXXRSRSRH | 18_470 | SXXXSXXXXXXXXXXH |
| 18_138 | XXXXXXXXXXSSSRRH | 18_497 | RRRSSRSSRSSRSRRH |
| 18_139 | XXXXXXXXXXSRSRSH | 18_498 | SSSRRSRRSRRSRSSH |
| 18_140 | SSRRRRSSSSSRSSRH | 18_499 | SRRSRSRSRRRSRRRH |
| 18_141 | SSSSSRRRRRRSRRSH | 18_500 | SRRRSSRRSSRSSSSH |
| 18_142 | SRSSRSSSRRRSRSRH | 18_501 | SRRRSSRSSRSRSSSH |
| 18_143 | SRRSSSSRRSRRRRRH | 18_502 | RRRSSRSRSSSRRRRH |
| 18_144 | SSRRSRSRSSSRSRRH | 18_503 | SRRRSSSRRRRSSSSH |
| 18_145 | SSSRRRRSRRRSSRRH | 18_504 | RRSSRSRSRSSRRSSH |
| 18_146 | RRSRSSRRSSSRRSSH | 18_505 | RRSRSRSRSSSRRSRH |
| 18_147 | RSSRRRSSSRSSSRSH | 18_506 | RSSSRRSSSRSRRSRH |
| 18_148 | SSSSRRRSRSSSRRSH | 18_507 | SRRSRSSSSSSRRRSH |
| 18_149 | SSSRSSSSSSSRRRRH | 18_508 | RRSSRSRRSRSRRRRH |
| 18_150 | SSSSRSSSSSSSSSSH | 18_509 | RRRRSRRRRSSSSRSH |
| 18_151 | RRSRRRRRSSSSSSSH | 18_510 | SSRRSRSRRSSSRRRH |
| 18_152 | RRRRSRSSRRRRSSSH | 18_511 | SSRRRRSRSSSRRRRH |
| 18_153 | RRRRRSSRRRSRSSRH | 18_512 | RRRRRSSSRSRSSSSH |
| 18_154 | SSRRRRSRSRSSRRSH | 18_513 | SRSRSSRRRSSSSSSH |
| 18_155 | RSSSSSRSSRRSSSSH | 18_514 | RSRSRSRSSRSRRRRH |
| 18_156 | RRRSSSSSRSRSRRSH | 18_515 | SSRRSRSSSSSRSSRH |
| 18_157 | RSSSRSRSRRRSRRRH | 18_516 | RSRRSRSSSSRRSSSH |
| 18_158 | RRSRRSSSRRRRRRSH | 18_517 | RRSSRSRRRSRRRSRH |
| 18_159 | RRSSSSRSRSSSRSRH | 18_518 | SRSRSSSSSSSSSSSH |
| 18_160 | RSSRSRSRSRSRSRRH | 18_519 | RSSSSSRSRSSSRSSH |
| 18_161 | SRRRSSSSRSRSRSRH | 18_520 | SRSSSSRSRSSSSRSH |
| 18_162 | SRSSSRRSRRRRSSRH | 18_521 | RRSRRSRRRSRRRSSH |
| 18_163 | RSSRRRSRRSRSSRRH | 18_522 | SRRSRSRSRSRSRRRH |
| 18_164 | SSRRRSSRSSRRRRSH | 18_523 | SRRRRSSSSRRSSRSH |
| 18_165 | RSRSSRRSRRRSSSRH | 18_524 | RSSSRRRRRSSSRRRH |
| 18_166 | RRRRSRRRSSRSRRSH | 18_525 | RRSSRRRRSSSSRRSH |
| 18_167 | SRRRSSSRSRSSRRRH | 18_526 | SSSSRSRRSRSSSRSH |
| 18_168 | SRSSRSSSSSRSRSSH | 18_527 | RRRRSRRSSSSSRSSH |
| 18_169 | SSRRSRSSSSSRSSSH | 18_528 | SRRSRSRRRRSSRRSH |
| 18_170 | SSRRRRRSRSRRSSSH | 18_529 | RSRSSRRRRRSSRSSH |
| 18_171 | SSSRRSSRSRRRRRSH | 18_530 | RRRSRSRSSRSRSSSH |
| 18_172 | RSSSSSSSRSRRRRRH | 18_531 | RRSSRSSSSSRSSSRH |
| 18_173 | SSRSRSSRSSRRSRRH | 18_532 | RRRSSSSSRSSSRSSH |
| 18_174 | SRSRSSSRRRSRRRSH | 18_533 | RRSSSSSRRSSRSRRH |
| 18_175 | RRRRRRRSSRRSSSRH | 18_534 | RSSRSRRSRSSSSRRH |
| 18_176 | SSRSRRRRRSRRSRSH | 18_535 | SSSSRSSSSRRSRRSH |
| 18_177 | RRSRRRRRRSSRRRSH | 18_536 | RRSSRRSSRSRRSSRH |
| 18_178 | SSSSRRRRRRRRRSRH | 18_537 | RRRSRRRRSSSRSSSH |
| 18_179 | SRRRSSRRRSSRRRSH | 18_538 | SSSRSSRRSRRRSSSH |
| 18_180 | SSSRRRRRSRRSSRRH | 18_539 | RSRRRRRRRSSSRRSH |
| 18_181 | RRSRRSSSSRRRSSRH | 18_540 | SSRSRSSSSRSRSRRH |
| 18_182 | SSRRSRSSRRRSSSSH | 18_541 | SSSRRSSSRSRRRRSH |
| 18_183 | SSRSRRRRSSRSSSRH | 18_542 | SSRRSSSSSRSRRSSH |
| 18_184 | RRRSRRSRSSRSRRRH | 18_543 | SSSRRRSRRRSSRSRH |
| 18_185 | RSRSSRSRSRRSRSRH | 18_544 | SRSSSSSRSSRSRRSH |
| 18_186 | SSSRRRRSSRRSRRRH | 18_545 | SRSSSSSSRRSSRRRH |
| 18_187 | RSSRRSRRRRSRRRSH | 18_546 | SRRSSSSRRRRRRSRH |
| 18_188 | SSSRRSSRSRSRSSSH | 18_547 | RSRSRRRSSSRSRRSH |
| 18_189 | RSRSSSSRSSRRRSSH | 18_548 | RRSRRSSSSSSSRSSH |
| 18_190 | SSSRSSSRSRRSRSSH | 18_549 | RSSRRRSSRRSSSSSH |
| 18_191 | RSSRSSSSRSSSSSRH | 18_550 | RSSRRSRSSRRSSRSH |
| 18_192 | RSSRRSSRSSSRRSRH | 18_551 | RRSSRSRRRRRRRRSH |
| 18_193 | RSSRRSRSRRSSSSRH | 18_552 | SRSSSRSRRRSSRSSH |
| 18_194 | RRSSSRRSRRRRSSSH | 18_553 | RSSRRRRRSRSRRRRH |
| 18_195 | RRRRRSSRSRRSSSRH | 18_554 | RSRSSSSRRSSSSSRH |
| 18_196 | SSSSRSRRRSSRRRSH | 18_555 | RRRRSSRRRSSRSSRH |
| 18_197 | RSRRRRRRRRSSRSRH | 18_556 | SSRSSRRSSSSRSRSH |
| 18_198 | RSRRSSSSRSSRSSRH | 18_557 | SRRRSSSSRRRSSRRH |
| 18_199 | SSRRSRSSRRRSSSRH | 18_558 | SRRSSSSRRSRRSRRH |
| 18_200 | RRRRSSSRRSRSRSSH | 18_559 | SSRRRSSRSSRSRRRH |
| 18_201 | RSRRRRRRSRRSSRSH | 18_560 | RSSRRRRSRSRRSRSH |
| 18_202 | SRRSRRRRRSRSSSSH | 18_561 | RSSRRRRSRRRRRRRH |
| 18_203 | SRRSRRSSSRSSSSSH | 18_562 | RRRRRRSRSRSRSSRH |
| 18_204 | SSSRRRRSRSRRRSSH | 18_563 | SSSRSSSSRRSSSRRH |
| 18_205 | SSRSRSRSSSRSRSRH | 18_564 | SRRSRSSSSSRSRRRH |
| 18_206 | SSSRRSRRSRRRSRSH | 18_565 | SSSSSRRSRSRSSRSH |
| 18_207 | SRSSRRRSSSSSRRRH | 18_566 | SSRSSRRSRRSSSRRH |
| 18_208 | RRSSRSSSSSSRSSRH | 18_567 | SSRSRSRRRSRSRRSH |
| 18_209 | SRSSRRSSRSRRSRRH | 18_568 | SRRSSRSRSRRRRSSH |
| 18_210 | RSRRSSRSRSSRRSSH | 18_569 | SRSRSRSRRSSSSRRH |
| 18_211 | RSSSRRSRSSSRSSSH | 18_570 | SRSSSRRRSRSSSSSH |
| 18_212 | SSSSSSSSRSRRRSSH | 18_571 | SRRSRSSSSSRSRSSH |
| 18_213 | RRSSSSSSSRSSSRRH | 18_572 | RSSRSRSRRSRSRRRH |
| 18_214 | SSSRRSSSSRRRRSSH | 18_573 | SSRSRRRRRRRSSSSH |
| 18_215 | SSSRRRRRRSSSSRRH | 18_574 | RRSSRRSSSSSSSSSH |
| 18_216 | RSRSRRRSSSRRRSRH | 18_575 | SRSSSRRRRRSSRSRH |
| 18_217 | SSSSRRSRRRSSRRRH | 18_576 | SSSSRSRRSSRRSRRH |
| 18_218 | RSSRRSSRSRRRSSSH | 18_577 | RSSSRSSRSRRRSSRH |
| 18_219 | RRSSSSSRRRRSRRSH | 18_578 | RRSRSRSRRRRSRRSH |
| 18_220 | RXXXXXXXXXXXXXXH | 18_579 | SRSRSSRSSSSSRRSH |
| 18_221 | SXXXXXXXXXXXXXXH | 18_580 | RRRSRRSSSSSSSRRH |
| 18_222 | XRXXXXXXXXXXXXXH | 18_581 | RRRSRSRSRSSRRRSH |
| 18_223 | XSXXXXXXXXXXXXXH | 18_582 | SSRRSRSSRRRRSSRH |
| 18_224 | XXRXXXXXXXXXXXXH | 18_583 | RRSSSSSRRRRSSRSH |
| 18_225 | XXSXXXXXXXXXXXXH | 18_584 | SRSSRRSRSSSRRSSH |
| 18_226 | XXXRXXXXXXXXXXXH | 18_585 | RSSSSSSRRSSSSRRH |
| 18_227 | XXXSXXXXXXXXXXXH | 18_586 | SRRRSSSSRRRSSSSH |
| 18_228 | XXXXRXXXXXXXXXXH | 18_587 | RRSRRRSRSSSSRSSH |
| 18_229 | XXXXSXXXXXXXXXXH | 18_588 | SSSSRSSSRSRSSSSH |
| 18_230 | XXXXXRXXXXXXXXXH | 18_589 | RRSRRRRRSRSSRSRH |
| 18_231 | XXXXXSXXXXXXXXXH | 18_590 | RRSSSRSRRRSRSSSH |
| 18_232 | XXXXXXRXXXXXXXXH | 18_591 | RRSRSRSSSRSSSSSH |
| 18_233 | XXXXXXSXXXXXXXXH | 18_592 | RRSSRSSSSRSRRSRH |
| 18_234 | XXXXXXXRXXXXXXXH | 18_593 | RRRRSSRSRSRSRSRH |
| 18_235 | XXXXXXXSXXXXXXXH | 18_594 | SRRSSRSSRRSRSSSH |
| 18_236 | XXXXXXXXRXXXXXXH | 18_595 | SRRSRRSRRRSSRSRH |
| 18_237 | XXXXXXXXSXXXXXXH | 18_596 | SSSSSRRRSSRRSSSH |
| 18_238 | XXXXXXXXXRXXXXXH | 18_597 | RRSRRRSRSSRSRRRH |
| 18_239 | XXXXXXXXXSXXXXXH | 18_598 | RSRSSRRSSRRSSRSH |
| 18_240 | XXXXXXXXXXRXXXXH | 18_599 | SSSRRRRSSRSRSSSH |
| 18_241 | XXXXXXXXXXSXXXXH | 18_600 | RRRRRSSRSRRRSRSH |
| 18_242 | XXXXXXXXXXXRXXXH | 18_601 | SSSRRSSRSRRSSRRH |
| 18_243 | XXXXXXXXXXXSXXXH | 18_602 | RRRSRSRSSRRSRRSH |
| 18_244 | XXXXXXXXXXXXRXXH | 18_603 | SRSSSSSRRSSRSRSH |
| 18_245 | XXXXXXXXXXXXSXXH | 18_604 | SSSRSSRSSSSSSSRH |
| 18_246 | XXXXXXXXXXXXXRXH | 18_605 | SSRSRSSRSSSSRRRH |
| 18_247 | XXXXXXXXXXXXXSXH | 18_606 | SRSRRSRRSRSRRRRH |
| 18_248 | XXXXXXXXXXXXXXRH | 18_607 | SRSRRRRSRSSRSSSH |
| 18_249 | XXXXXXXXXXXXXXSH | 18_608 | SRSRRRRRSSSRRSRH |
| 18_362 | SSSSSSSRRXXXXXXH | 18_609 | RRRSSSSRSSRRSSRH |
| 18_363 | SSRSSSSRRXXXXXXH | 18_610 | RRRSSSSSRRSRSRRH |
| 18_364 | SSSRSSSRRXXXXXXH | | |
| | | | Parent Oligonucleotide Cmp/ |
| SEQ ID NO | Design | CMP ID NO | stereodefinition |
| 18 | 1-1-2-8-4 | 18_347 | TcAActttcactTCAG |
| | | | XXXXXXXXXXXXXXXH |
| CMP ID NO | Stereodefined motif | CMP ID NO | Stereodefined motif |
| 18_471 | SSSRRSSSRRRRRSSH | 18_478 | SSSRSSSRSRRSRSSH |
| 18_472 | XXXXRSSRXXXXXXXH | 18_479 | SRRSRSRSRRRSRRRH |
| 18_473 | XXXXXXXXXXRSSSRH | 18_480 | SRRRSSRRSSRSSSSH |
| 18_474 | XXXXXXXXXRRSRRSH | 18_481 | SRRRSSRSSRSRSSSH |
| 18_475 | SSSSRSRRRSSRRRSH | 18_482 | RRRSSRSRSSSRRRRH |
| 18_476 | RRSRSSRRSSSRRSSH | 18_483 | SRRRSSSRRRRSSSSH |
| 18_477 | RSRSSSSRSSRRRSSH | | |
| | | | Parent Oligonucleotide Cmp/ |
| SEQ ID NO | Design | CMP ID NO | stereodefinition |
| 18 | 3-9-4 | 18_12 | TCAactttcactTCAG |
| | | | XXXXXXXXXXXXXXXH |
| CMP ID NO | Stereodefined motif | CMP ID NO | Stereodefined motif |
| 18_484 | SSSRRSSSRRRRRSSH | 18_491 | SSSSRSRRRSSRRRSH |
| 18_485 | XXXXRSSRXXXXXXXH | 18_492 | SRRSRSRSRRRSRRRH |
| 18_486 | XXXXXXXXXXRSSSRH | 18_493 | SRRRSSRRSSRSSSSH |
| 18_487 | XXXXXXXXXRRSRRSH | 18_494 | SRRRSSRSSRSRSSSH |
| 18_488 | RRSRSSRRSSSRRSSH | 18_495 | RRRSSRSRSSSRRRRH |
| 18_489 | RSRSSSSRSSRRRSSH | 18_496 | SRRRSSSRRRRSSSSH |
| 18_490 | SSSRSSSRSRRSRSSH | | |

In relation to the parent oligonucleotide CMP: Capital letters represent beta-D-oxy LNA nucleosides, lowercase letters represent DNA nucleosides, all LNA C are 5-methyl cytosine, all internucleoside linkages are phosphorothioate internucleoside linkages.

In relation to the stereodefinition/stereodefined motifs: X represent a stereorandom phosphorothioate internucleoside linkage, R represents one stereoisomeric form and S represents the other stereoisomeric form as defined in the a description, H represents the hydrogen atom at the 3′ terminus of the oligonucleotide. The first letter (X, R or S) in the stereodefined motif correspond to the internucleoside linkage between nucleoside 1 and 2 from the 5′ end of the oligonucleotide.

TABLE 9
Oligonucleotide motif sequences and antisense
compounds with 5′ ca biocleavable
linker.

| | | oligonucleotide compound | |
|---|---|---|---|
| | | with a C6 alkyl ca | CMP |
| SEQ ID NO | motif sequence | biocleavable linker | ID NO |
| 20 | CATCAACTTTCACTTCAG | C6ₒcₒaₒTCAactttcacttCAG | 20_1 |
| 20 | CATCAACTTTCACTTCAG | C6ₒcₒaₒTCAActttcactTCAG | 20_2 |
| 20 | CATCAACTTTCACTTCAG | C6ₒcₒaₒTCAActttcacttCAG | 20_3 |
| 20 | CATCAACTTTCACTTCAG | C6ₒcₒaₒTCAActttcacTtCAG | 20_4 |
| 20 | CATCAACTTTCACTTCAG | C6ₒcₒaₒTCAACtttcacttCAG | 20_5 |
| 20 | CATCAACTTTCACTTCAG | C6ₒcₒaₒTCAACtttcacttcAG | 20_6 |
| 20 | CATCAACTTTCACTTCAG | C6ₒcₒaₒTCAActttcacttcAG | 20_7 |
| 20 | CATCAACTTTCACTTCAG | C6ₒcₒaₒTCAactttcactTCAG | 20_8 |
| 20 | CATCAACTTTCACTTCAG | C6ₒcₒaₒTcAACtttcactTcAG | 20_9 |
| 20 | CATCAACTTTCACTTCAG | C6ₒcₒaₒTcAACtttcacttcAG | 20_10 |
| 20 | CATCAACTTTCACTTCAG | C6ₒcₒaₒTCaACtttcacttcAG | 20_11 |
| 20 | CATCAACTTTCACTTCAG | C6ₒcₒaₒTCaActttcacttCAG | 20_23 |
| 20 | CATCAACTTTCACTTCAG | C6ₒcₒaₒTCaactttcactTCAG | 20_24 |
| 20 | CATCAACTTTCACTTCAG | C6ₒcₒaₒTCAaCtttcacttCAG | 20_25 |
| 20 | CATCAACTTTCACTTCAG | C6ₒcₒaₒTCaaCtttcacttCAG | 20_26 |
| 20 | CATCAACTTTCACTTCAG | C6ₒcₒaₒTCAaCtttcacttcAG | 20_27 |
| 20 | CATCAACTTTCACTTCAG | C6ₒcₒaₒTCaActttcactTCAG | 20_28 |
| 20 | CATCAACTTTCACTTCAG | C6ₒcₒaₒTcAActttcactTCAG | 20_29 |
| 20 | CATCAACTTTCACTTCAG | C6ₒcₒaₒTCAActttcactTcAG | 20_37 |
| 20 | CATCAACTTTCACTTCAG | C6ₒcₒaₒTcaACtttcacttCAG | 20_38 |
| 21 | CATCAACTTTCACTTCAGT | C6ₒcₒaₒTCAActttcacttCaGT | 21_1 |
| 21 | CATCAACTTTCACTTCAGT | C6ₒcₒaₒTcAactttcactTcAGT | 21_3 |
| 21 | CATCAACTTTCACTTCAGT | C6ₒcₒaₒTcAActttcacttCaGT | 21_4 |
| 21 | CATCAACTTTCACTTCAGT | C6ₒcₒaₒTCAactttcacttcAGT | 21_5 |
| 21 | CATCAACTTTCACTTCAGT | C6ₒcₒaₒTCaactttcacTtCAGT | 21_6 |
| 21 | CATCAACTTTCACTTCAGT | C6ₒcₒaₒTCAactttcacTtCaGT | 21_7 |
| 21 | CATCAACTTTCACTTCAGT | C6ₒcₒaₒTCaActttcactTCAGT | 21_8 |
| 21 | CATCAACTTTCACTTCAGT | C6ₒcₒaₒTcAActttcactTCAGT | 21_9 |
| 21 | CATCAACTTTCACTTCAGT | C6ₒcₒaₒTCAActttcactTCaGT | 21_10 |
| 21 | CATCAACTTTCACTTCAGT | C6ₒcₒaₒTCAactttcactTCaGT | 21_11 |
| 21 | CATCAACTTTCACTTCAGT | C6ₒcₒaₒTcAActttcactTCaGT | 21_12 |
| 21 | CATCAACTTTCACTTCAGT | C6ₒcₒaₒTCaactttcactTcAGT | 21_13 |
| 21 | CATCAACTTTCACTTCAGT | C6ₒcₒaₒTCAactttcacttCAGT | 21_14 |
| 21 | CATCAACTTTCACTTCAGT | C6ₒcₒaₒTCaactttcacttCAGT | 21_15 |
| 21 | CATCAACTTTCACTTCAGT | C6ₒcₒaₒTcAActttcacttCAGT | 21_16 |
| 21 | CATCAACTTTCACTTCAGT | C6ₒcₒaₒTcAactttcacttCAGT | 21_17 |
| 21 | CATCAACTTTCACTTCAGT | C6ₒcₒaₒTCAactttcacttCaGT | 21_18 |
| 21 | CATCAACTTTCACTTCAGT | C6ₒcₒaₒTCAActttcacttcAGT | 21_19 |
| 21 | CATCAACTTTCACTTCAGT | C6ₒcₒaₒTCaactttcactTCAGT | 21_37 |
| 21 | CATCAACTTTCACTTCAGT | C6ₒcₒaₒTCaActttcactTCaGT | 21_38 |
| 21 | CATCAACTTTCACTTCAGT | C6ₒcₒaₒTCAActttcactTcaGT | 21_39 |
| 21 | CATCAACTTTCACTTCAGT | C6ₒcₒaₒTCaActttcacttCAGT | 21_40 |

C6 represents an amino alkyl group with 6 carbons, capital letters represent beta-D-oxy LNA nucleosides, lowercase letters represent DNA nucleosides, all LNA C are 5-methyl cytosine, subscript o represent a phosphodiester internucleoside linkage and unless otherwise indicated other internucleoside linkages are phosphorothioate internucleoside linkages.

TABLE 10
GalNAc conjugated antisense oligonucleotide
compounds.

| | | | Corre- |
|---|---|---|---|
| | | | sponding |
| | CMP | antisense | CMP ID |
| SEQ ID | ID | oligonucleotide | of naked |
| NO | NO | conjugate | compound |
| 20 | 20_12 | GN2-C6ₒcₒaₒTCAactttcacttCAG | 18_1 |
| 20 | 20_13 | GN2-C6ₒcₒaₒTCAActttcactCAG | 10_10 |
| 20 | 20_14 | GN2-C6ₒcₒaₒTCAActttcacttCAG | 18_19 |
| 20 | 20_15 | GN2-C6ₒcₒaₒTCAActttcacTtCAG | 18_5 |
| 20 | 20_16 | GN2-C6ₒcₒaₒTCAACtttcacttCAG | 18_18 |
| 20 | 20_17 | GN2-C6ₒcₒaₒTCAACtttcacttcAG | 18_23 |
| 20 | 20_18 | GN2-C6ₒcₒaₒTCAActttcacttcAG | 18_24 |
| 20 | 20_19 | GN2-C6ₒcₒaₒTCAactttcactTCAG | 18_12 |
| 20 | 20_20 | GN2-C6ₒcₒaₒTcAACtttcactTcAG | 18_15 |
| 20 | 20_21 | GN2-C6ₒcₒaₒTcAACtttcacttcAG | 18_30 |
| 20 | 20_22 | GN2-C6ₒcₒaₒTCaACtttcacttcAG | 18_27 |
| 20 | 20_30 | GN2-C6ₒcₒaₒTCaActttcacttCAG | 18_357 |
| 20 | 20_31 | GN2-C6ₒcₒaₒTCaactttcactTCAG | 18_14 |
| 20 | 20_32 | GN2-C6ₒcₒaₒTCAaCtttcacttCAG | 18_20 |
| 20 | 20_33 | GN2-C6ₒcₒaₒTCaaCtttcacttCAG | 18_21 |
| 20 | 20_34 | GN2-C6ₒcₒaₒTCAaCtttcacttcAG | 18_25 |
| 20 | 20_35 | GN2-C6ₒcₒaₒTCaActttcactTCAG | 18_346 |
| 20 | 20_36 | GN2-C6ₒcₒaₒTcAActttcactTCAG | 18_347 |
| 20 | 20_39 | GN2-C6ₒcₒaₒTCAActttcactTcAG | 18_350 |
| 20 | 20_40 | GN2-C6ₒcₒaₒTcaACtttcacttCAG | 18_358 |
| 21 | 21_2 | GN2-C6ₒcₒaₒTCAActttcacttCaGT | 17_10 |
| 21 | 21_20 | GN2-C6ₒcₒaₒTcAactttcactTcAGT | 17_7 |
| 21 | 21_21 | GN2-C6ₒcₒaₒTcAAcfficacttCaGT | 17_13 |
| 21 | 21_22 | GN2-C6ₒcₒaₒTCAactttcacttcAGT | 17_14 |
| 21 | 21_23 | GN2-C6ₒcₒaₒTCaactttcacTtCAGT | 17_51 |
| 21 | 21_24 | GN2-C6ₒcₒaₒTCAactttcacTtCaGT | 17_57 |
| 21 | 21_25 | GN2-C6ₒcₒaₒTCaActttcactTCAGT | 17_86 |
| 21 | 21_26 | GN2-C6ₒcₒaₒTcAActttcactTCAGT | 17_90 |
| 21 | 21_27 | GN2-C6ₒcₒaₒTCAActttcactTCaGT | 17_96 |
| 21 | 21_28 | GN2-C6ₒcₒaₒTCAactttcactTCaGT | 17_99 |
| 21 | 21_29 | GN2-C6ₒcₒaₒTcAActttcactTCaGT | 17_103 |
| 21 | 21_30 | GN2-C6ₒcₒaₒTCaactttcactTcAGT | 17_111 |
| 21 | 21_31 | GN2-C6ₒcₒaₒTCAactttcacttCAGT | 17_129 |
| 21 | 21_32 | GN2-C6ₒcₒaₒTCaactttcacttCAGT | 17_135 |
| 21 | 21_33 | GN2-C6ₒcₒaₒTcAActttcacttCAGT | 17_137 |
| 21 | 21_34 | GN2-C6ₒcₒaₒTcAactttcacttCAGT | 17_139 |
| 21 | 21_35 | GN2-C6ₒcₒaₒTCAactttcacttCaGT | 17_144 |
| 21 | 21_36 | GN2-C6ₒcₒaₒTCAActttcacttcAGT | 17_157 |
| 21 | 21_41 | GN2-C6ₒcₒaₒTCaactttcactTCAGT | 17_89 |
| 21 | 21_42 | GN2-C6ₒcₒaₒTCaActttcactTCaGT | 17_100 |
| 21 | 21_43 | GN2-C6ₒcₒaₒTCAActttcactTcaGT | 17_119 |
| 21 | 21_44 | GN2-C6ₒcₒaₒTCaActttcacttCAGT | 17_132 |

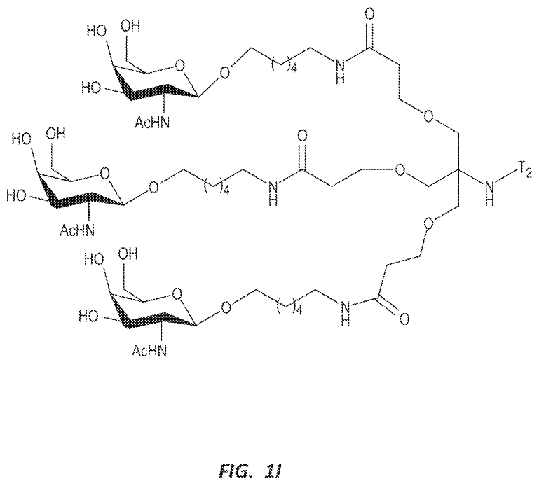

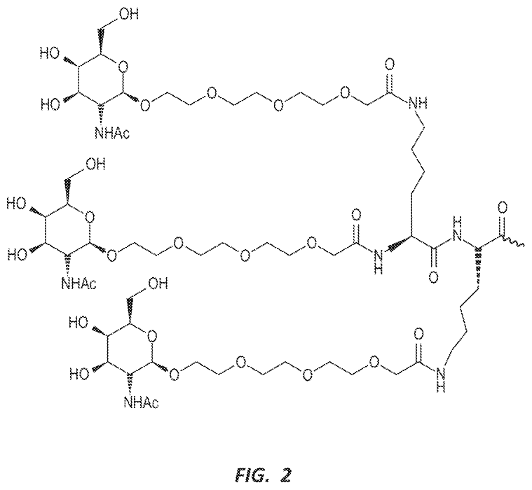

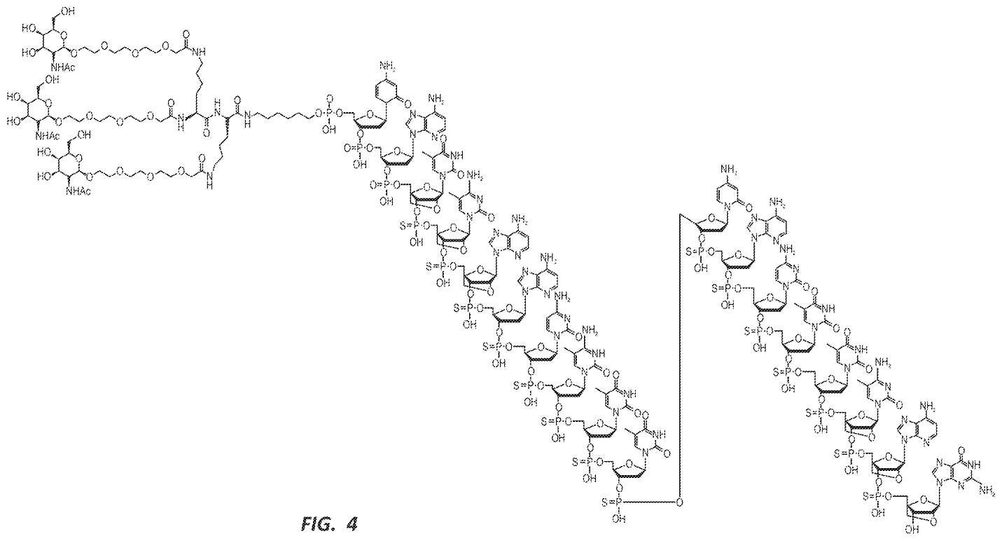

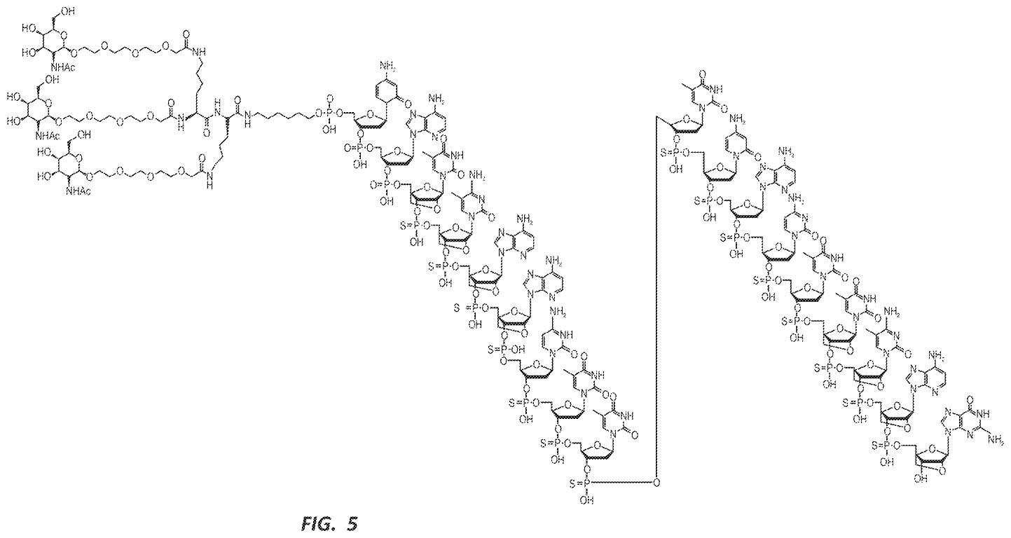

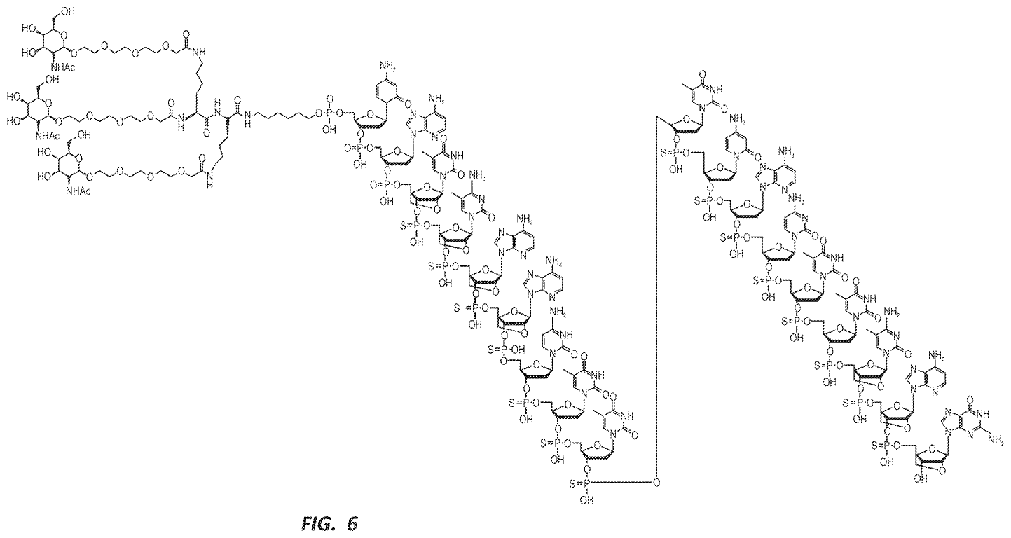

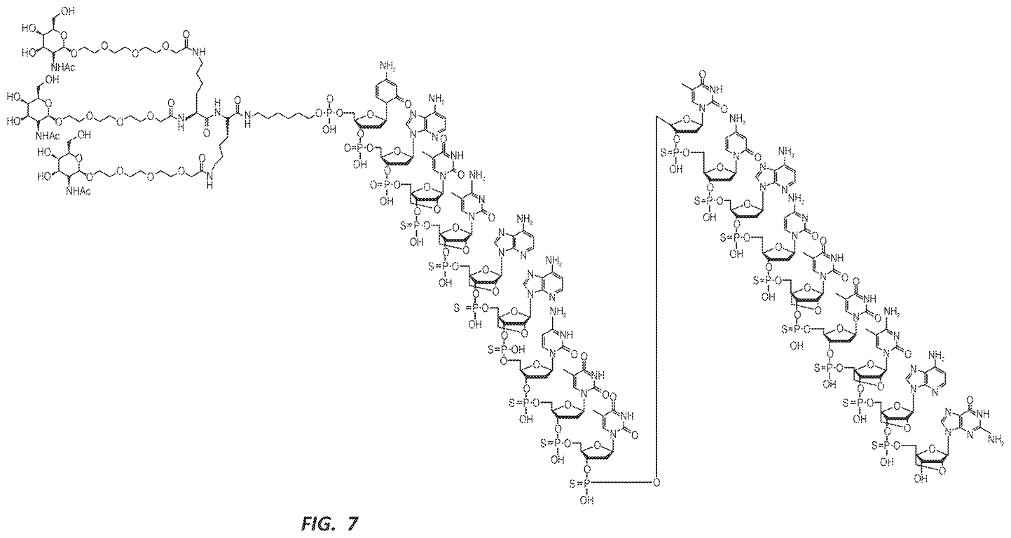

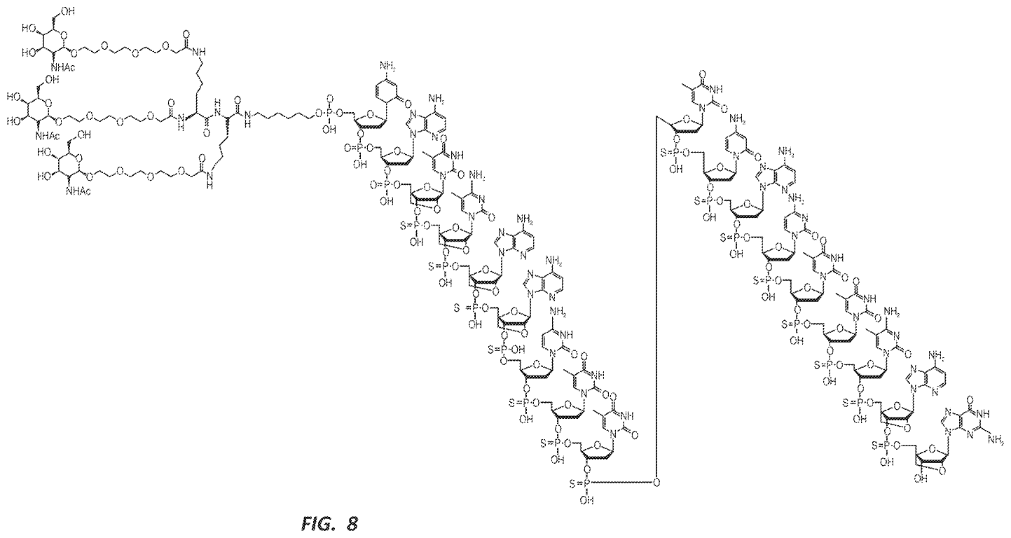

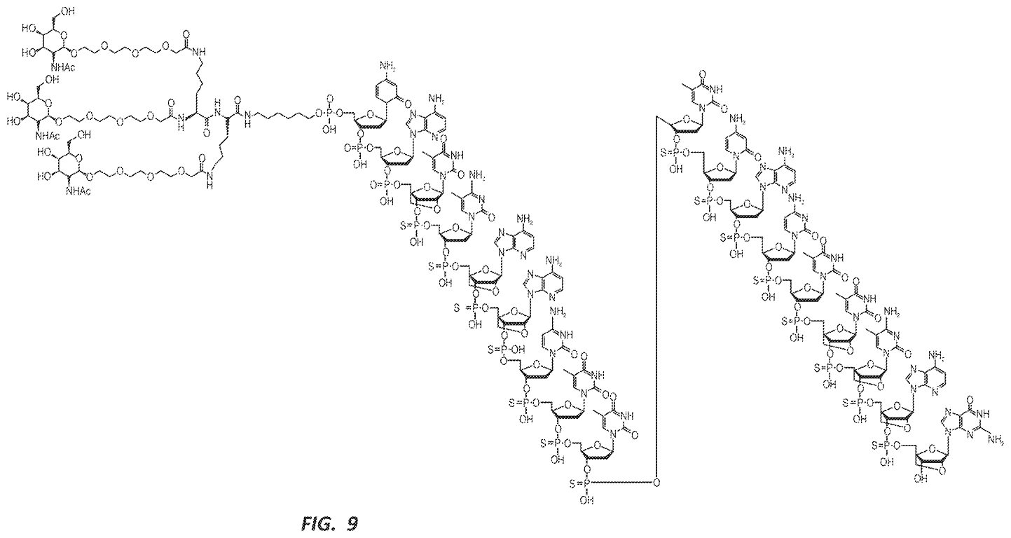

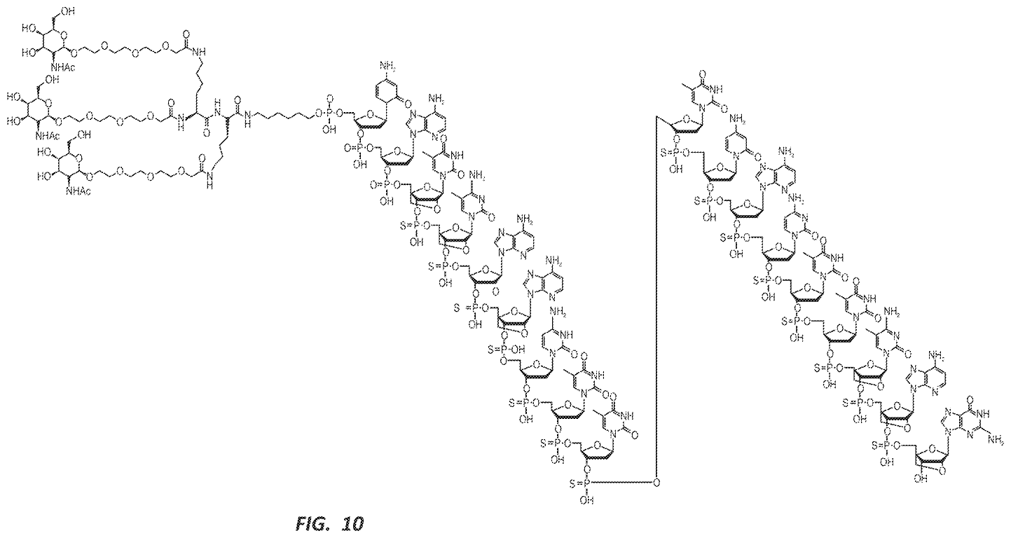

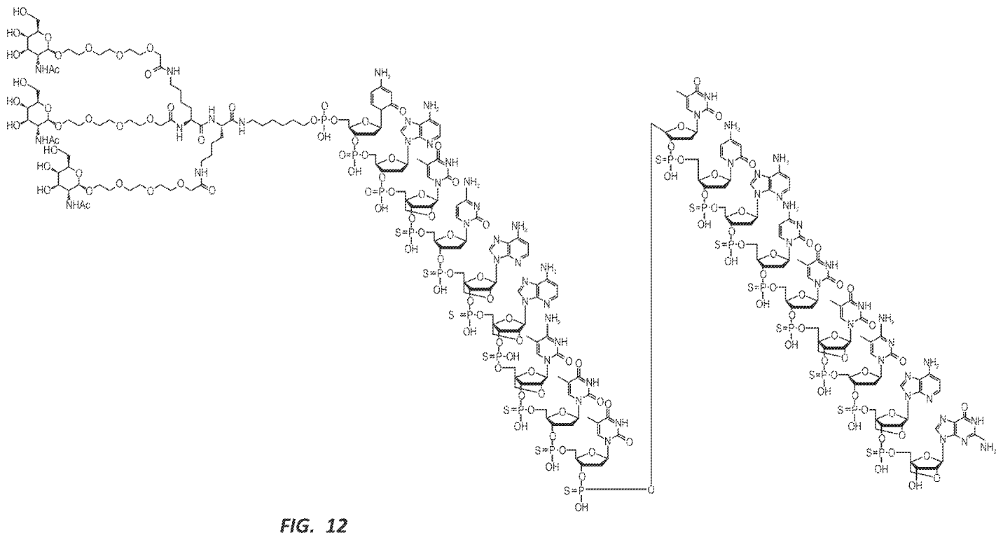

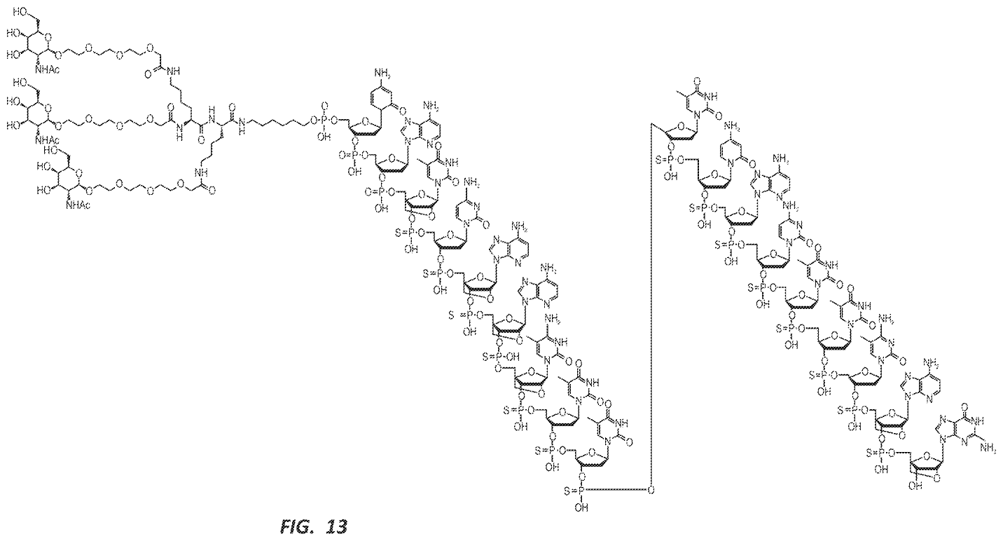

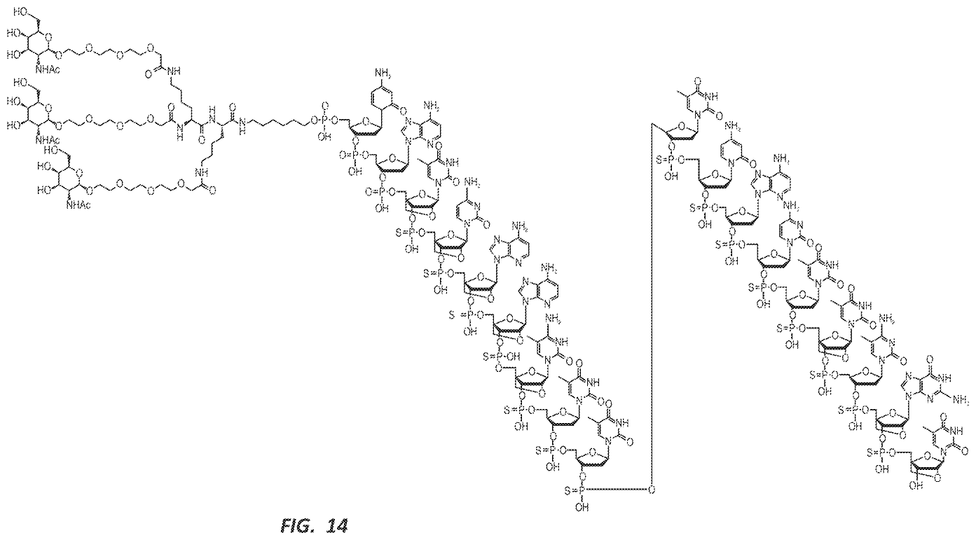

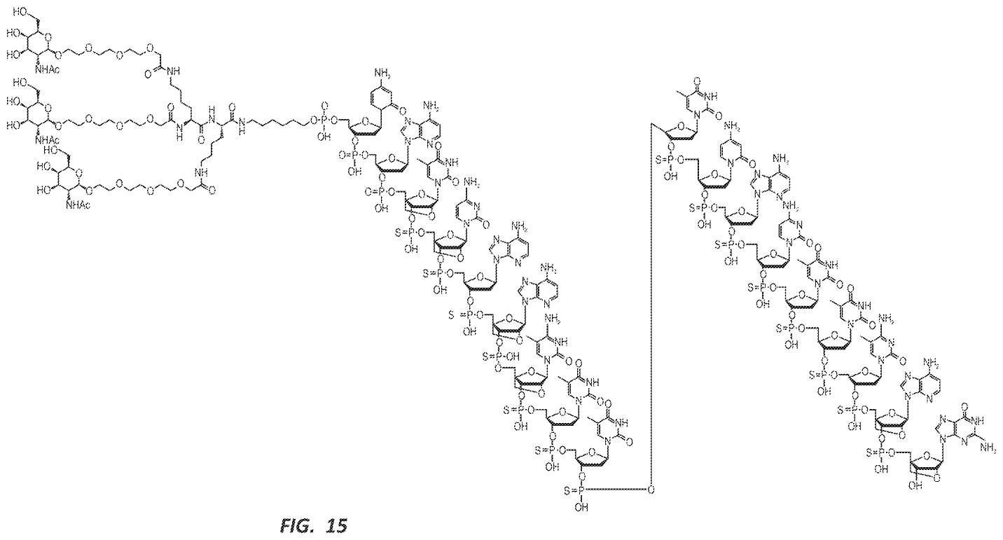

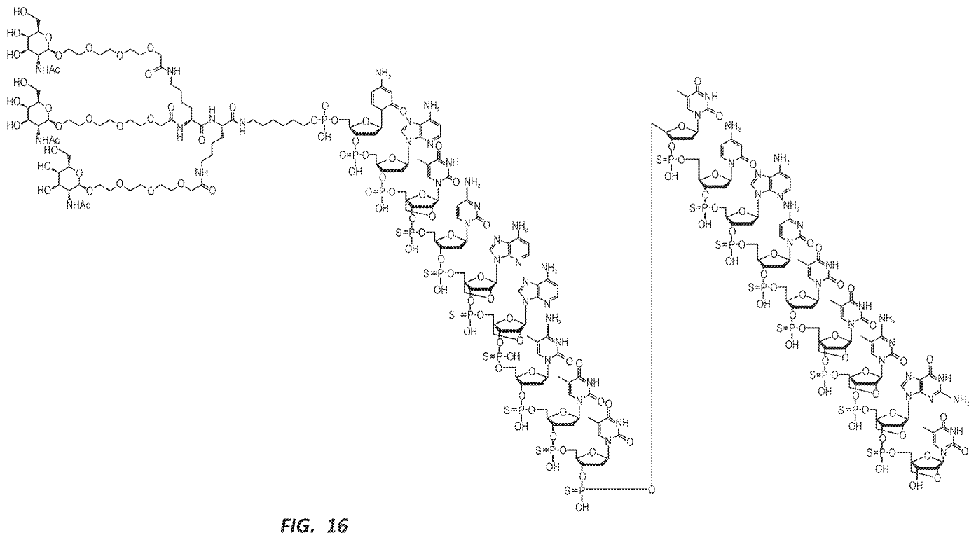

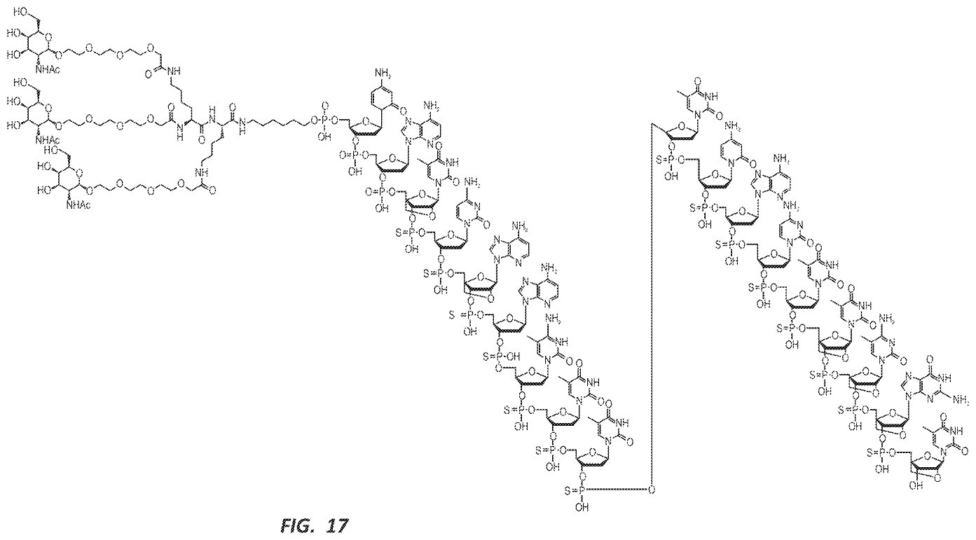

GN2 represents the trivalent GalNAc cluster shown in FIG. 2, C6 represents an amino alkyl group with 6 carbons, capital letters represent beta-D-oxy LNA nucleosides, lowercase letters represent DNA nucleosides, all LNA C are 5-methyl cytosine, subscript o represent a phosphodiester nucleoside linkage and unless otherwise indicated internucleoside linkages are phosphorothioate internucleoside linkages. Chemical drawings representing some of the molecules are shown in FIGS. 4 to 17.

### AAW/HBV Mouse Models

In the AAV/HBV mouse model mice are infected with a recombinant adeno-associated virus (AAV) carrying the HBV genome (AAV/HBV) maintains stable viremia and antigenimia for more than 30 weeks (Dan Yang, et al. 2014 Cellular & Molecular Immunology 11, 71-78).

Male C57BL/6 mice (4-6 weeks old), specific pathogen free, are purchased from SLAC (Shanghai Laboratory Animal Center of Chinese Academy of Sciences) and housed in an animal care facility in individually ventilated cages. Guidelines are followed for the care and use of animals as indicated by WuXi IACUC (Institutional Animal Care and Use Committee, WUXI IACUC protocol number R20131 126-Mouse). Mice are allowed to acclimate to the new environment for 3 days and are grouped according to the experimental design.

Recombinant AAV-HBV is diluted in PBS, 200 μL per injection. This recombinant virus carries 1.3 copies of the HBV genome (genotype D, serotype ayw).

On day 0, all mice are injected through tail vein with 200 μL AAV-HBV (1×10¹¹ vector genome). On Pre-dose Day 23 (23 days post AAV-HBV injection), animals were distributed to in groups based on serum levels of HBV markers and body weight. Each group was housed (up to 5/cage) in polycarbonate cages with corncob bedding. Low, medium, and high HBV titer values were spread, ensuring group means to be similar across groups. The animal groups can be treated with oligonucleotides which can be unconjugated or GalNAc conjugated. All serum collections (0.1 ml blood/mouse) were performed by retro-orbital bleeding after animals were anesthetized with isoflurane inhalation.

### HeLa Cell Lines

HeLa cell line was purchased from European Collection of Authenticated Cell Cultures (ECACC, #93021013) and maintained as recommended by the supplier in a humidified incubator at 37° C. with 5% CO2. For assays, 2,500 cells/well were seeded in a 96 multi well plate in Eagle's Minimum Essential Medium (Sigma, M2279) with 10% fetal bovine serum (FBS), 2 mM Glutamin AQ, 1% NEAA, 25 μg/ml Gentamicin.

### Differentiated HepaRG Cell Culture (No HBV Infection)

HepaRG cells (Biopredics International, Rennes, France, Cat #HPR101) were cultured at 37° C. in a humidified atmosphere with 5% CO2 in complete HepaRG growth medium consisting of William's E Medium (Sigma W4128), Growth Medium Supplement (Biopredics, Cat #ADD710) and 1% (v/v) GlutaMAX-I (Gibco #32551) for 2 weeks.

To initiate differentiation cells were grown in complete HepaRG growth medium for 2 weeks until they were fully confluent. Half of the medium was exchanged by HepaRG differentiation medium consisting of William's E Medium (Sigma W4128), Growth Medium Supplement (Biopredics, Cat #ADD720) and 1% (v/v) GlutaMAX-1 (Gibco #32551), final concentration of DMSO was 0.9% (v/v)). After 3 days, medium was fully replaced by complete differentiation medium (final concentration of DMSO 1.8% (v/v)) in which cells were maintained for approximately 2 weeks with differentiation medium renewal every 7 days. Differentiated HepaRG cells (dHepaRG), displayed hepatocyte-like cell islands surrounded by monolayer of biliary-like cells. Prior to compound treatment, dHepaRG cells were seeded into collagen I coated 96-well plates (Corning BioCoat REF354407) at 80,000 cells per well in 100 μL of complete differentiation medium. Cells were allowed to recover their differentiated phenotype in 96-well plates for approximately 1 week after plating prior to oligonucleotide treatment. RNA was isolated 6 days after treatment.

HBV Infected dHepaRG Cells

HepaRG cells (Biopredics International, Rennes, France, Cat #HPR101) were cultured at 37° C. in a humidified atmosphere with 5% CO2 in complete HepaRG growth medium consisting of William's E Medium (GIBCO), Growth Medium Supplement (Biopredics, Cat #ADD711C) and 1% (v/v) GlutaMAX-I (Gibco #32551) and 1×Pen/Strep (Gibco, #15140) for 2 weeks.

To initiate differentiation, 0.9% (v/v) DMSO (Sigma-Aldrich, D2650) was added to the growth medium on confluent cells. After one week, medium was replaced by complete differentiation medium (HepaRG growth medium supplemented with 1.8% (v/v) DMSO) in which cells were maintained for approximately 4 weeks with differentiation medium renewal every 7 days. Differentiated HepaRG cells (dHepaRG), displayed hepatocyte-like cell islands surrounded by monolayer of biliary-like cells.

Prior to HBV infection and compound treatment, dHepaRG cells were seeded into collagen I coated 96-well plates (Gibco, Cat #A11428-03) at 60,000 cells per well in 100 μL of complete differentiation medium. Cells were allowed to recover their differentiated phenotype in 96-well plates for approximately 1 week after plating prior to HBV infection.

The dHepaRG cells were infected with HBV particles at an MOI of 30. The HBV particles were produced from HBV-producing HepG2.2.15 cells (Sells et al 1987 Proc Natl Acad Sci USA 84, 1005-1009). dHepaRG culture conditions, differentiation and HBV infection have been described previously (Hantz, 2009, J. Gen. Virol., 2009, 90: 127-135). In brief complete differentiation medium (HepaRG growth medium consisting of William's E Medium (GIBCO), Growth Medium Supplement (Biopredics, Cat #ADD711C) and 1% (v/v) GlutaMAX-I (Gibco #32551) and 1×Pen/Strep (Gibco, #15140), supplemented with 1.8% (v/v) DMSO), containing 4% PEG-8000 and virus stock (20 to 30 GE/cell) was added (120 μL/well). One day post-infection, the cells were washed four times with phosphate-buffered saline and medium (complete differentiation medium) was replaced on day 4 and day 7 during the experiment.

### HBV Infected ASGPR-dHepaRG

From the HepaRG cell line (Biopredics International, Rennes, France, Cat #HPR101) a cell line stably overexpressing human ASGPR1 and ASGPR2 was generated using a lentiviral method. Proliferating HepaRG cells were transduced at MOI 300 with a lentivirus produced on demand by Sirion biotech (CLV-CMV-ASGPR1-T2a_ASGPR2-IRES-Puro) coding for Human ASGPR1 and 2 under the control of a CMV promoter and a puromycin resistance gene. Transduced cells were selected for 11 days with 1 μg/ml puromycin and then maintained in the same concentration of antibiotic to ensure stable expression of the transgenes. ASGPR1/2 overexpression was confirmed both at mRNA level by RT-qPCR (ASGPR1: 8560 fold vs non-transduced, ASGPR2: 2389 fold vs non-transduced), and at protein level by flow cytometry analysis. The differentiated cells are termed ASGPR-dHepaRG cells.

The ASGPR-HepaRG cells were differentiated using 1.8% DMSO for at least 2 weeks before infection. HBV infection was performed as for the dHepaRG cells described above.

### Primary Mouse Hepatocytes (PMH)

Primary mouse hepatocytes were isolated from livers of C57BL/6J mice anesthetized with Pentobarbital after a 2 step perfusion protocol according to the literature (Berry and Friend, 1969, J. Cell Biol; Paterna et al., 1998, Toxicol. Appl. Pharmacol.). The first step was 5 min with HBSS+15 mM HEPES+0.4 mM EGTA followed by 12 min HBSS+20 mM NaHCO₃+0.04% BSA (Sigma #A7979)+4 mM CaCL₂ (Sigma #21115)+0.2 mg/ml Collagenase Type 2 (Worthington #4176). The Hepatocytes were captured in 5 ml cold Williams medium E (WME) (Sigma #W1878, complemented with 1×Pen/Strep/Glutamine, 10% (v/v) FBS (ATCC #30-2030)) on ice.

The crude cell suspension was filtered through a 70 μm followed by a 40 μm cell strainer (Falcon #352350 and #352340), filled up to 25 ml with WME and centrifuged at room temperature for 5 min at 50×g to pellet the hepatocytes. The supernatant was removed and the hepatocytes were resuspended in 25 ml WME. After adding 25 ml 90% Percoll solution (Sigma #P4937; pH=8.5-9.5) and centrifugation for 10 min at 25° C., 50×g the supernatant and floating cells were removed. To remove the remaining Percoll the pellet was resuspended again in 50 mL WME medium, centrifuged 3 min, 25° C. at 50×g and the supernatant discarded. The cell pellet was resuspended in 20 mL WME and cell number and viability determined (Invitrogen, Cellcount) and diluted to 250,000 cells/ml. 25,000 cells/well were seeded on collagen-coated 96-well plates (PD Biocoat Collagen I #356407) and incubated at 37° C., 5% CO₂. After 3-4 h, the cells were washed with WME to remove unattached cells and the medium was replaced. 24 h after seeding the oligonucleotides were added in the desired concentration and the cells were incubated at 37° C., 5% CO2 for 72 hours. RNA isolation (Qiagen, RNeasy 96) was followed by one-step RT-QPCR (Quanta Bioscience, qScript XLT 1-Step RT-qPCR ToughMix) using TaqMan assays for the target genes (PAPD5:Mm01244121_m1 FAM-MGB, PAPD7: Mm01349513_m1 FAM-MGB) and a house keeping gene (GusB Mm_01197698_m1, VIC-MGB) according to the manufacturer's protocols.

### Primary Human Hepatocyte (PHH) Natural Infection Assay

Primary human hepatocytes (PHH) isolated by collagenase perfusion method from chimeric uPA/SCID mice with humanized livers were obtained from PhoenixBio (Hiroshima, Japan). The cells were plated on type I collagen coated 96-well plates at a concentration of 7×104 cells per well in culture media provided by Phoenix Bio (See Ishida et al 2015 Am J Pathol. Vol 185 page1275-1285 for further details). HBV genotype D was derived from HepG2.2.15 cell culture supernatant and concentrated using PEG precipitation. PHHs were infected in PHH medium containing 4% PEG 8000 at MOI 10 for 20 h at 37° C. before cells were washed 4 times with PBS. One day 1 post-infection, oligonucleotide was delivered to the cells in a final volume of 125 μl of PHH medium. The cells were retreated on day 4 and 7 post-infection. At day 11 post-infection, supernatants and cells were harvested. HBsAg and HBeAg levels in the supernatants were assessed using the CLIA ELISA assay (see Materials and Method section; HBV antigen measurements). mRNA was extracted from the cells using a MagNA Pure robot and the MagNA Pure 96 Cellular RNA Large Volume Kit (Roche, #05467535001) according to the manufacturer's protocol. The relative PAPD5 and PAPD7 mRNA expression levels were analyzed using Real-time PCR as described in Materials and Methods section.

### HBV Antigen Measurements

To evaluate the impact on HBV antigen expression and secretion, supernatants were collected on Day 11. The HBV propagation parameters, HBsAg and HBeAg levels, were measured using CLIA ELISA Kits (Autobio Diagnostic #CL0310-2, #CL0312-2), according to the manufacturer's protocol. Briefly, 25 μL of supernatant per well were transferred to the respective antibody coated microtiter plate and 25 μL of enzyme conjugate reagent were added. The plate was incubated for 60 min on a shaker at room temperature before the wells were washed five times with washing buffer using an automatic washer. 25 μL of substrate A and B were added to each well. The plates were incubated on a shaker for 10 min at room temperature before luminescence was measured using an Envision luminescence reader (Perkin Elmer).

Real-Time PCR for Intracellular HBV mRNA from HBV Infected Cells

HBV mRNA was quantified in technical duplicate by qPCR using a QuantStudio 12K Flex (Applied Biosystems), the TaqMan RNA-to-CT 1-Step Kit (Applied Biosystems, #4392938), Human ACTB endogenous control (Applied Biosystems, #4310881E). Taqman reagents were used together with the following commercial ThermoFisher Sceintific primers (HBV Pa03453406_s1, ACTB 4310881E). The mRNA expression was analyzed using the comparative cycle threshold 2-ΔΔCt method normalized to the reference gene ACTB and to PBS treated cells.

Real-Time PCR for PAPD5 and PAPD7 mRNA Expression

QPCR was conducted on RNA extracted from treated cells or homogenized tissue samples. After RNA/LNA duplex denaturation (90° C., 40 sec) Real-time PCR was done with a one-step protocol (qScript™ XLT One-Step RT-qPCR ToughMix®, Low ROX™ from Quanta Bioscience, #95134-500) in a duplex set up with the following TaqMan primer assays (ThermoFisher Scientific):
PAPD5 (Hs00223727_m1, FAM-MGB)
PAPD7 (Hs00173159_m1, FAM-MGB),

House keeping gene GUSB (Hu_4326320E, VIC-MGB) following the recommendations of the provider.

### HBV DNA Quantification Viral Particle Titer

HBV DNA extraction is performed using the QIAamp UltraSens Virus kit (Qiagen, #53704) according to the manufacturer's protocol with the following optimizations. 30 μL and 3 μL of the virus sample are diluted into 1 mL of PBS before adding buffer AC. The first centrifugation step is done for 45 min at full speed and 4° C. HBV DNA is quantified in duplicate by qPCR using a QuantStudio 12K Flex (Applied Biosystems), the TaqMan Gene Expression Master Mix (Applied Biosystems, #4369016) and a premix 1:1:0.5 of the primers indicated in Table 9 above and probe reconstituted at 100 μM. The qPCR is performed using the following settings: UDG incubation (2 min, 50° C.), enzyme activation (10 min, 95° C.) and qPCR (40 cycles with 15 sec, 95° C. for denaturation and 1 min, 60° C. for annealing and extension). Genomes equivalent calculation is based on a standard curve generated from HBV genotype D plasmid dilutions with known concentrations.

The HBV particle titer can be determined using HBV core-specific primer (Integrated DNA Technologies) (Table 11) in a QPCR on isolated intracellular mRNA from treated cells.

TABLE 11
HBV core specific TaqMan probes

| | | | | SEQ ID |
|---|---|---|---|---|
| | Name | Dye | Sequence | NO |
| HBV | Forward | | CTG TGC CTT GGG TGG | 24 |
| core | (F3_HBVcore) | | CTT T | |
| Primer | Reverse | | AAG GAA AGA AGT CAG | 25 |
| | (R3_HBVcore) | | AAG GCA AAA | |
| | Probe | FAM- | AGC TCC AAA/ZEN/TTC | 26 |
| | (P3_HBVcore) | MGB | TTT ATA AGG GTC GAT | |
| | | | GTC CAT G | |

ZEN is an internal quencher

### Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

### Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle, a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Tₘ Assay:

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Example 1: Screening for In Vitro Efficacy of Antisense Oligonucleotides Targeting PAPD5 And PAPD7 (Bispecific) in HeLa Cells

An oligonucleotide screen was done using 16 to 18mer gapmers targeting SEQ ID NO: 17, 18 and 19. Efficacy testing was performed in an in vitro experiment in HeLa cells expressing both PAPD5 and PAPD7.

HeLa cells were cultured as described in the Materials and Method section. The cells were incubated for 24 hours before addition of oligonucleotides dissolved in PBS. Final concentration of oligonucleotides was 5 and 25 μM, the final culture volume was 100 μl/well. The cells were harvested 3 days after addition of oligonucleotide compounds and RNA was extracted using the PureLink Pro 96 RNA Purification kit (Ambion), according to the manufacturer's instructions.

PAPD5 and PAPD7 mRNA levels were analysed by Real-time PCR as described in the Materials and Method section.

The relative PAPD5 mRNA and PAPD7 mRNA expression levels are shown in table 12 as % of average control samples (PBS-treated cells) i.e. the lower the value the larger the inhibition.

TABLE 12
in vitro efficacy of anti-PAPD5/PAPD7 compounds (single experiment
with duplex QPCR). PAPD5 and PAPD7 mRNA levels are normalized to
GUSB in HeLa cells and shown as % of control (PBS treated cells).

| | % PAPD5 mRNA of control | | | | % PAPD7 mRNA of control | | | | |
|---|---|---|---|---|---|---|---|---|---|
| CMP ID | 25 μM | | 5 μM | | 25 μM | | 5 μM | | |
| NO | Avg | sd | Avg | sd | Avg | sd | Avg | sd | Compound (CMP) |
| 17_2 | 35.36 | 0.58 | 69.86 | 3.08 | 31.55 | 0.88 | 89.02 | 14.48 | TCaaCtttcacTtcAGT |
| 17_3 | 13.76 | 1.40 | 35.71 | 3.94 | 11.56 | 1.63 | 56.65 | 11.86 | TCaactttcacTtcAGT |
| 17_4 | 39.72 | 2.23 | 51.51 | 4.97 | 83.29 | 11.18 | 117.6 | 14.81 | TcaaCtttcacTtcAGT |
| 17_5 | 24.87 | 2.09 | 53.56 | 8.57 | 62.21 | 2.96 | 27.92 | 2.32 | TCaactttcacTtcaGT |
| 17_6 | 19.50 | 1.22 | 34.68 | 0.37 | 14.51 | 0.16 | 82.74 | 26.43 | TCaaCtttcactTCaGT |
| 17_7 | 6.17 | 1.04 | 22.09 | 0.01 | 13.47 | 3.64 | 20.41 | 3.12 | TcAactttcactTcAGT |
| 17_8 | 9.85 | 1.44 | 28.15 | 4.60 | 25.29 | 4.47 | 26.39 | 3.48 | TcAActttcactTcaGT |
| 17_9 | 18.73 | 2.57 | 47.62 | 3.48 | 31.00 | 3.51 | 58.02 | 6.32 | TCAACtttcacttCaGT |
| 17_10 | 6.13 | 1.18 | 23.39 | 0.44 | 5.88 | 0.34 | 31.76 | 3.25 | TCAActttcacttCaGT |
| 17_11 | 14.04 | 2.09 | 31.58 | 4.40 | 42.82 | 6.50 | 86.43 | 11.95 | TCaaCtttcacttCaGT |
| 17_12 | 15.33 | 0.62 | 29.82 | 1.07 | 34.94 | 5.35 | 51.77 | 3.89 | TCaactttcacttCaGT |
| 17_13 | 6.63 | 0.34 | 23.62 | 9.01 | 8.49 | 0.51 | 20.44 | NA | TcAActttcacttCaGT |
| 17_14 | 4.61 | 1.98 | 22.51 | 5.00 | 6.19 | 0.36 | 44.27 | 6.69 | TCAactttcacttcAGT |
| 17_15 | 17.99 | 2.70 | 32.73 | 4.67 | 26.59 | 2.61 | 38.30 | 4.15 | TCaaCtttcacttcAGT |
| 17_16 | 42.29 | 1.06 | 75.49 | 6.32 | 26.91 | 1.57 | 46.19 | 0.88 | TCaactttcacttcaGT |
| 18_2 | 41.16 | 0.15 | 65.30 | 5.51 | 48.83 | 6.29 | 63.37 | 10.84 | TCaaCtttcacTTCAG |
| 18_3 | 54.39 | 3.08 | 71.95 | 2.89 | 69.99 | 0.89 | 66.50 | 3.56 | TcAACtttcacTTcAG |
| 18_4 | 40.86 | 1.32 | 64.99 | 4.39 | 78.13 | 1.60 | 109.0 | 0.49 | TCAACtttcacTtCAG |
| 18_5 | 9.30 | 0.76 | 27.26 | 0.91 | 7.32 | 1.32 | 14.80 | 1.92 | TCAActttcacTtCAG |
| 18_6 | 7.49 | 0.75 | 21.64 | 2.49 | 10.32 | 0.39 | 14.16 | 0.82 | TCaactttcacTtCAG |
| 18_7 | 25.02 | 0.30 | 47.25 | 4.07 | 37.93 | 10.34 | 68.66 | 5.11 | TcaaCtttcacTtCAG |
| 18_8 | 22.93 | 8.09 | 44.18 | 1.59 | 33.95 | 7.34 | 39.70 | 5.06 | TCaActttcacTtcAG |
| 18_9 | 15.21 | 2.21 | 39.74 | 0.32 | 12.21 | 1.80 | 23.08 | 0.01 | TCAACtttcactTCAG |
| 18_10 | 3.99 | 0.67 | 20.53 | 4.40 | 7.81 | 0.52 | 23.89 | 2.49 | TCAActttcactTCAG |
| 18_11 | 13.84 | 3.93 | 35.46 | 1.52 | 28.39 | 1.96 | 56.56 | 11.43 | TCAaCtttcactTCAG |
| 18_12 | 5.13 | 0.14 | 20.21 | 0.24 | 3.40 | 0.29 | 41.51 | 7.20 | TCAactttcactTCAG |
| 18_13 | 11.90 | 1.05 | 26.20 | 0.47 | 26.51 | 0.82 | 20.79 | 5.61 | TCaaCtttcactTCAG |
| 18_14 | 5.42 | 0.33 | 20.05 | 2.62 | 8.85 | 1.46 | 66.72 | 8.16 | TCaactttcactTCAG |
| 18_15 | 7.16 | 0.03 | 20.84 | 1.94 | 6.17 | 0.05 | 46.67 | 1.26 | TcAACtttcactTcAG |
| 18_16 | 14.28 | 2.44 | 33.79 | 1.00 | 29.49 | 1.95 | 16.87 | 2.38 | TcAaCtttcactTcAG |
| 18_17 | 27.49 | 2.66 | 61.62 | 9.21 | 55.71 | 3.61 | 36.14 | 0.32 | TcaaCtttcactTcAG |
| 18_18 | 5.43 | 0.61 | 26.45 | 0.75 | 3.16 | 0.61 | 35.64 | 2.03 | TCAACtttcacttCAG |
| 18_19 | 4.85 | 1.04 | 17.24 | 1.69 | 12.48 | 0.60 | 13.12 | 0.88 | TCAActttcacttCAG |
| 18_20 | 5.51 | 0.05 | 20.28 | 1.07 | 12.76 | 1.24 | 14.83 | 0.13 | TCAaCtttcacttCAG |
| 18_21 | 10.64 | 0.32 | 23.88 | 1.67 | 12.61 | 0.50 | 14.50 | 1.05 | TCaaCtttcacttCAG |
| 18_22 | 10.66 | 1.95 | 34.29 | 7.33 | 16.22 | 1.84 | 25.81 | 7.43 | TCaactttcacttCAG |
| 18_23 | 5.50 | 1.99 | 24.63 | 0.61 | 10.97 | 0.12 | 27.22 | 1.51 | TCAACtttcacttcAG |
| 18_24 | 8.37 | 0.44 | NA | NA | 12.02 | 1.77 | NA | NA | TCAActttcacttcAG |
| 18_25 | 7.58 | 0.80 | 23.71 | 3.32 | 9.03 | 0.05 | 19.79 | 1.14 | TCAaCtttcacttcAG |
| 18_26 | 12.94 | 0.46 | 35.03 | 2.99 | 25.90 | 0.06 | 28.01 | 0.45 | TCAactttcacttcAG |
| 18_27 | 7.21 | 1.46 | 21.24 | 2.15 | 19.27 | 2.92 | 72.92 | 25.73 | TCaACtttcacttcAG |
| 18_28 | 15.47 | 4.10 | 39.98 | 4.60 | 14.80 | 0.36 | 43.25 | 5.37 | TCaaCtttcacttcAG |
| 18_29 | 32.76 | 9.68 | 43.53 | 4.96 | 21.47 | 5.16 | 34.84 | 0.17 | TCaactttcacttcAG |
| 18_30 | 4.45 | 0.12 | 20.61 | 5.21 | 10.94 | 1.63 | 24.09 | 0.58 | TcAACtttcacttcAG |
| 18_31 | 55.81 | 9.87 | 71.92 | 22.31 | 50.86 | 4.18 | 60.22 | 0.42 | TcaaCtttcacttcAG |
| 19_1 | 101.9 | 10.60 | 89.66 | 13.79 | 59.35 | 6.51 | 160.6 | 2.10 | TGTTTcaatacTAAAA |
| 19_2 | 90.94 | 1.54 | 68.65 | 6.91 | 59.66 | 1.75 | 60.33 | 1.98 | TGTTtcaatacTAAAA |
| 19_3 | 104.6 | 13.82 | 86.79 | 12.54 | 80.71 | 0.60 | 68.25 | 5.99 | TGTTTcaatacTAaAA |

### Example 2: In Vitro EC50 and Efficacy in HBV Infected HepaRG Cells

All the oligonucleotides from Example 1 were tested for their effect on HBV propagation parameters in HBV infected dHepaRG cells.

For comparative purposes the antisense oligonucleotides of the invention were compared to antisense oligonucleotides targeting HBV mRNA directly. The HBV targeting oligonucleotides are shown in table 13.

TABLE 13
Comparative HBV targeting oligonucleotides

| | | SEQ ID | |
|---|---|---|---|
| Description | Compound | NO | Reference |
| HBV targeting | AGCgaagtgcacaCGG | 27 | WO2015/173208 |
| 1 (HBV1) | | | |
| HBV targeting | GCGtaaagagaGG | 28 | WO2015/173208 |
| 2 (HBV2) | | | |

HBV infected dHepaRG cells (described in the Materials and Methods section, HBV infected dHepaRG cells) were cultured in 96-well plates. One day post HBV infection the oligonucleotides were added to the cells in three-fold serial dilutions (20.00, 6.67, 2.22, 0.74, 0.25, 0.08, 0.03, 0.01 μM oligonucleotide) using unassisted uptake (gymnosis). A total of 49 oligonucleotides were tested. The experiment was conducted in triplicate, with PBS controls. The oligonucleotide treatment was repeated at day 4 and 7.

At day 11 post-infection, supernatants and cells were harvested.

HBsAg and HBeAg levels in the supernatants were assessed using the CLIA ELISA assay (see Materials and Methods, HBV antigen measurements).

EC 50, max KD (efficacy) of the HBV propagation parameters HBsAg and HBeAg was calculated using the R-function drm( ) from the drc package (v3.0-1) a four-parameter log-logistic function is fitted to the expression of the gene of interest as a function of oligonucleotide concentration to obtain a value for EC50 and maximum knock-down. The results are shown in table 14 and are % of average control samples (PBS control and Non infected (NIF), calculated as follows [(Test Value−meanPBS)/(meanNIF−meanPBS)]*100)).

TABLE 14
EC50 and Max KD of anti-PAPD5/PAPD7 compounds on HBsAg and HBeAg
(average of 3) in HBV infected dHepaRG cells.

| | HBsAg | | | | HBeAg | | | | |
|---|---|---|---|---|---|---|---|---|---|
| CMP | Max KD | | EC50 | | Max KD | | EC50 | | |
| ID | % of saline | | μM | | % of saline | | μM | | |
| NO | Avg | sd | Avg | sd | Avg | sd | Avg | sd | Compound |
| 17_7 | 57.18 | 6.67 | 7.36 | 20.66 | 33.61 | 10.44 | 7.07 | 15.94 | TcAactttcacTcCAGT |
| 17_8 | 28.29 | 13.46 | 4.75 | 1.59 | 23.75 | 11.32 | 5.14 | 1.69 | TcAActttcactTcaGT |
| 17_10 | 19.10 | 4.81 | 6.73 | 15.00 | 2.28 | 11.52 | 6.63 | 2.67 | TCAActttcacttCaGT |
| 17_13 | 22.07 | 8.55 | 5.74 | 1.01 | 4.09 | 15.51 | 4.40 | 1.52 | TcAActttcacttCaGT |
| 17_14 | 0.00 | 855.97 | 24.07 | 61.33 | 1.04 | NA | 21.37 | NA | TCAactttcacttcAGT |
| 18_1 | 5.42 | 9.05 | 4.67 | 0.71 | 5.88 | 14.10 | 4.12 | 1.22 | TCAactttcacttCAG |
| 18_5 | 4.70 | 9.40 | 6.67 | 1.20 | 0.30 | 7.04 | 4.86 | 0.80 | TCAActttcacTtCAG |
| 18_6 | 26.99 | 12.22 | 6.66 | 1.39 | 22.14 | 9.60 | 6.40 | 3.64 | TCaactttcacTtCAG |
| 18_10 | 0.00 | 10.01 | 4.94 | 0.88 | 2.68 | 10.92 | 4.40 | 1.09 | TCAActttcactTCAG |
| 18_12 | 14.01 | 8.21 | 6.52 | 0.60 | 3.86 | 14.96 | 6.12 | 1.14 | TCAactttcactTCAG |
| 18_15 | 15.87 | 25.90 | 6.22 | 3.82 | 32.23 | 7.88 | 2.10 | 4.75 | TcAACtttcactTCAG |
| 18_18 | 8.11 | 11.24 | 7.21 | 1.14 | 8.75 | 6.36 | 6.58 | 5.28 | TCAACtttcacttCAG |
| 18_19 | 3.43 | 3.49 | 2.32 | 0.18 | 3.75 | 5.69 | 2.16 | 3.09 | TCAActttcacttCAG |
| 18_20 | 36.72 | 4.45 | 7.05 | 17.16 | 0.00 | 74.91 | 8.07 | 9.71 | TCAaCtttcacttCAG |
| 18_21 | 26.03 | 51.79 | 9.16 | 9.36 | 0.00 | 92.94 | 10.13 | 14.18 | TCaaCtttcacttCAG |
| 18_23 | 11.13 | 7.74 | 5.53 | 0.76 | 6.33 | 9.42 | 4.82 | 0.99 | TCAACtttcacttcAG |
| 18_24 | 11.95 | 8.90 | 3.64 | 0.82 | 13.90 | 10.15 | 2.36 | 0.62 | TCAActttcacttcAG |
| 18_25 | 25.93 | 17.79 | 7.90 | 2.60 | 19.84 | 10.18 | 6.78 | 4.08 | TCAaCtttcacttcAG |
| 18_30 | 16.85 | 5.93 | 2.51 | 0.38 | 12.47 | 8.12 | 2.22 | 0.27 | TcAACtttcacttcAG |
| 17_3 | 93.91 | 127.26 | 32.39 | 329.47 | 89.14 | 8.47 | 0.91 | 10.00 | TCaactttcacTtcAGT |
| 17_5 | 90.80 | 7.82 | 1.31 | 10.00 | 95.11 | 10.13 | 0.10 | 10.00 | TCaactttcacTtcaGT |
| 17_6 | 92.43 | NA | 0.57 | NA | 89.80 | NA | 0.00 | NA | TCaaCtttcactTCaGT |
| 17_9 | 54.71 | 6.03 | 7.08 | 14.69 | 15.37 | 35.83 | 8.44 | 3.80 | TCAACtttcacttCaGT |
| 17_11 | 83.26 | 7.52 | 3.61 | 10.00 | 62.66 | 9.37 | 0.58 | 10.00 | TCaaCtttcacttCaGT |
| 17_12 | 97.35 | 7.36 | 19.89 | 10.00 | 78.78 | 8.65 | 0.35 | 10.00 | TCaactttcacttCaGT |
| 17_15 | 91.43 | NA | 0.67 | NA | 78.81 | 8.76 | 0.46 | 10.00 | TCaaCtttcacttcAGT |
| 18_7 | 90.45 | NA | 11.53 | NA | 85.05 | 8.27 | 0.34 | 10.00 | TcaaCtttcacTtCAG |
| 18_8 | 63.76 | 12.80 | 5.22 | 1.98 | 52.50 | 9.20 | 4.77 | 1.14 | TCaActttcacTtAG |
| 18_9 | 23.40 | 156.35 | 12.06 | 23.00 | 26.07 | 11.37 | 7.57 | 16.01 | TCAACtttcactTCAG |
| 18_11 | 0.00 | 236.59 | 23.95 | 50.46 | 0,05 | NA | 18.25 | NA | TCAaCtttcactTCAG |
| 18_13 | 53.81 | 6.31 | 7.16 | 11.60 | 42.15 | 8.15 | 7.31 | 13.89 | TCaaCtttcactTCAG |
| 18_14 | 32.71 | 11.10 | 5.13 | 1.25 | 24.27 | 14.19 | 4.20 | 1.31 | TCaactttcactTCAG |
| 18_16 | 81.65 | 6.89 | 7.15 | 17.43 | 72.67 | 8.30 | 7.01 | 9.77 | TcAaCtttcactTcAG |
| 18_22 | 29.19 | 5.87 | 6.40 | 7.22 | 16.60 | 18.52 | 4.54 | 1.31 | TCaactttcacttCAG |
| 18_26 | 40.75 | 8.16 | 5.35 | 0.90 | 36.63 | 6.43 | 5.34 | 1.09 | TCAactttcacttcAG |
| 18_27 | 20.92 | 10.83 | 4.61 | 1.10 | 13.89 | 13.63 | 4.03 | 1.20 | TCaACtttcacttcAG |
| 18_28 | 67.96 | 9.83 | 8.11 | 77.37 | 47.21 | 2274.28 | 18.70 | 138.89 | TCaaCtttcacttcAG |
| 17_2 | 84.70 | 14.17 | 0.28 | 10.00 | 61.86 | 9.52 | 0.21 | 10.00 | TCaaCtttcacTtcAGT |
| 17_4 | 85.48 | 10.18 | 0.31 | 10.00 | 55.95 | 9.53 | 0.13 | 10.00 | TcaaCtttcacTtcAGT |
| 17_16 | 68.31 | 10.41 | 0.10 | 10.00 | 39.65 | 9.69 | 0.27 | 10.00 | TCaactttcacttcaGT |
| 18_2 | 94.41 | 8.20 | 0.47 | 10.00 | 61.03 | 9.43 | 0.28 | 10.00 | TCaaCtttcacTTCAG |
| 18_3 | 68.72 | 9.16 | 0.24 | 10.00 | 51.03 | 9.02 | 0.14 | 10.00 | TcAACtttcacTTcAG |
| 18_4 | 92.84 | 8.61 | 0.12 | 10.00 | 85.97 | 8.77 | 0.18 | 10.00 | TCAACtttcacTtCAG |
| 18_17 | 71.76 | 8.21 | 0.59 | 10 00 | 49.14 | 8.82 | 0.83 | 10.00 | TcaaCtttcactTcAG |
| 18_29 | 81.88 | 9.30 | 1.00 | 10.00 | 72.13 | 9.16 | 0.24 | 10.00 | TCaactttcacttcAG |
| 18_31 | 73.12 | 9.07 | 0.43 | 10.00 | 73.76 | 8.47 | 0.47 | 10.00 | TcaaCtttcacttcAG |
| 19_1 | 82.69 | 9.37 | 0.20 | 10.00 | 96.30 | 10.43 | 0.06 | 10.00 | TGTTTcaatacTAAAA |
| 19_2 | 85.50 | 16.76 | 0.27 | 10.00 | 83.38 | 8.96 | 0.24 | 10.00 | TGTTtcaatacTAAAA |
| 19_3 | 103.91 | NA | 0.30 | NA | 108.39 | 8.81 | 0.09 | 10.00 | TGTTTcaatacTAaAA |
| HBV1 | 0.00 | 16.32 | 2.44 | 1.22 | 0.00 | 23.37 | 1.33 | 1.09 | AGCgaagtgcacaCGG |
| HBV2 | 0.00 | 55.69 | 16.80 | 19.97 | 0.00 | NA | 20.73 | NA | GCGtaaagagaGG |

From these data it can be seen that a significant number of the compounds have a good effect on HBsAg and HBeAg. Compounds with the oligonucleotide motif of SEQ ID NO 17 and 18 seem more efficient than the compounds that have been made with the motif of SEQ ID NO: 19

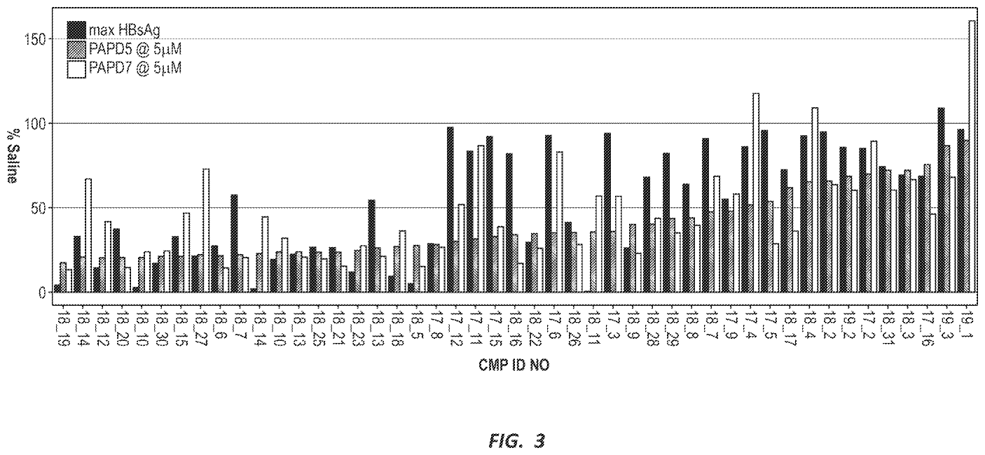

In FIG. 3, it can also be seen that for oligonucleotides that reduce PAPD5 and PAPD7 in HeLa cells with more than 70% there is a high correlation with respect to these oligonucleotides ability to reduce HBsAg in HBV infected dHepaRG cells.

### Example 3 Screening for In Vitro Efficacy of Antisense Oligonucleotides Targeting PAPD5 And PAPD7 in HeLa Cells

A further library of 298 oligonucleotides expanding the diversity of the oligonucleotide motifs of SEQ ID NO: 17, 18 and 19 using different designs was generated. Efficacy testing was performed in an in vitro experiment as described in Example 1, with the exception that the screening was only conducted at 5 μM.

The relative PAPD5 mRNA and PAPD7 mRNA expression levels are shown in table 15 as % of average control samples (PBS-treated cells) i.e. the lower the value the larger the inhibition.

TABLE 15
in vitro efficacy of anti-PAPD5/PAPD7 compounds
(single experiment with duplex QPCR). PAPD5 and
PAPD7 mRNA levels are normalized to GUSB in
HeLa cells and shown as % of control (PBS treated
cells).

| | % | | % | | |
|---|---|---|---|---|---|
| | PAPD5 mRNA | | PAPD7 mRNA | | |
| | of control | | of control | | |
| CMP ID | 5 μM | | 5 μM | | |
| NO | Avg | sd | Avg | sd | Compound (CMP) |
| 17_17 | 97.74 | 7.10 | 88.55 | 3.38 | TCAaCtttcacTTCAGT |
| 17_18 | 86.48 | 5.52 | 81.81 | 1.73 | TCaACtttcacTTCAGT |
| 17_19 | 66.13 | 13.83 | 78.41 | 1.05 | TCaaCtttcacTTCAGT |
| 17_20 | 62.79 | 2.79 | 61.90 | 1.55 | TCaactttcacTTCAGT |
| 17_21 | 86.77 | 5.77 | 84.45 | 2.79 | TcAACtttcacTTCAGT |
| 17_22 | 83.56 | 9.69 | 76.97 | 2.27 | TcAAcTttcacTTCAGT |
| 17_23 | 75.81 | 5.73 | 73.23 | 5.44 | TcaaCtttcacTTCAGT |
| 17_24 | 97.11 | NA | 88.80 | 2.14 | TCAACtttcacTTCaGT |
| 17_25 | 62.02 | 5.46 | 64.52 | 2.73 | TCAActttcacTTCaGT |
| 17_26 | 90.95 | 11.41 | 92.31 | 2.78 | TCAaCtttcacTTCaGT |
| 17_27 | 75.23 | 6.15 | 75.70 | 3.92 | TCAacTttcacTTCaGT |
| 17_28 | 57.34 | 11.56 | 51.15 | 2.33 | TCAactttcacTTCaGT |
| 17_29 | 86.07 | 8.22 | 79.21 | 4.63 | TCaACtttcacTTCaGT |
| 17_30 | 82.66 | 3.99 | 82.55 | 7.92 | TCaAcTttcacTTCaGT |
| 17_31 | 63.66 | 7.08 | 58.10 | 6.16 | TCaActttcacTTCaGT |
| 17_32 | 70.24 | 8.96 | 74.38 | 4.15 | TCaaCtttcacTTCaGT |
| 17_33 | 62.01 | 4.54 | 66.85 | 2.18 | TCaacTttcacTTCaGT |
| 17_34 | 47.04 | 1.05 | 53.40 | 3.12 | TCaactttcacTTCaGT |
| 17_35 | 77.50 | 7.79 | 79.78 | 1.36 | TcAACtttcacTTCaGT |
| 17_36 | 100.06 | 11.65 | 81.00 | 3.56 | TCAACtttcacTTcAGT |
| 17_37 | 85.23 | 8.93 | 80.34 | 2.60 | TCAAcTttcacTTcAGT |
| 17_38 | 68.09 | 6.84 | 70.24 | 2.54 | TCaAcTttcacTTcAGT |
| 17_39 | 75.83 | 14.88 | 74.95 | 1.29 | TcAAcTttcacTTcAGT |
| 17_40 | 60.89 | 6.53 | 69.40 | 1.14 | TcaAcTttcacTTcAGT |
| 17_41 | 67.33 | 12.02 | 73.92 | 1.59 | TcaaCtttcacTTcAGT |
| 17_42 | 55.60 | 7.22 | 68.28 | 1.86 | TcaacTttcacTTcAGT |
| 17_43 | NA | NA | 73.73 | 6.69 | TcAACtttcacTTcaGT |
| 17_44 | 78.69 | 9.83 | 69.98 | 3.35 | TcAaCtttcacTTcaGT |
| 17_45 | 76.31 | 5.75 | 77.93 | 6.73 | TcaaCtttcacTTcaGT |
| 17_46 | 82.77 | 4.94 | 88.62 | 3.06 | TCAACtttcacTtCAGT |
| 17_47 | 75.09 | 3.28 | 75.56 | NA | TCAaCtttcacTtCAGT |
| 17_48 | 41.87 | 3.23 | 46.58 | 4.31 | TCaActttcacTtCAGT |
| 17_49 | 65.39 | 3.03 | 73.12 | 4.72 | TCaaCtttcacTtCAGT |
| 17_50 | 44.54 | 7.92 | 58.99 | 1.91 | TCaacTttcacTtCAGT |
| 17_51 | 38.28 | 4.62 | 49.61 | 11.12 | TCaactttcacTtCAGT |
| 17_52 | 72.04 | 11.74 | 67.18 | 1.56 | TcaaCtttcacTtCAGT |
| 17_53 | 77.11 | 6.61 | 80.39 | 4.87 | TCAACtttcacTtCaGT |
| 17_54 | 68.58 | 5.17 | 81.14 | 9.92 | TCAAcTttcacTtCaGT |
| 17_55 | 54.70 | NA | 55.71 | 7.63 | TCAActttcacTtCaGT |
| 17_56 | 73.62 | 8.99 | 77.13 | 4.24 | TCAaCtttcacTtCaGT |
| 17_57 | 37.11 | 4.10 | 45.26 | 2.67 | TCAactttcacTtCaGT |
| 17_58 | 75.70 | 7.51 | 79.77 | 3.37 | TCaACtttcacTtCaGT |
| 17_59 | 62.77 | 7.89 | 67.67 | 2.31 | TCaAcTttcacTtCaGT |
| 17_60 | 59.08 | 5.30 | 53.75 | 3.07 | TCaActttcacTtCaGT |
| 17_61 | 58.34 | 2.53 | 66.25 | 3.04 | TCaaCTttcacTtCaGT |
| 17_62 | 69.33 | 5.17 | 72.06 | 2.78 | TCaaCtttcacTtCaGT |
| 17_63 | 61.54 | NA | 64.88 | 2.78 | TCaacTttcacTtCaGT |
| 17_64 | 49.47 | 3.41 | 50.89 | 2.55 | TCaactttcacTtCaGT |
| 17_65 | 80.85 | 11.35 | 81.88 | 4.86 | TCAACtttcacTtcAGT |
| 17_66 | 65.22 | NA | 68.32 | 2.12 | TCAAcTttcacTtcAGT |
| 17_67 | 54.53 | 4.81 | 53.80 | 1.98 | TCAActttcacTtcAGT |
| 17_68 | 74.51 | 6.00 | 76.56 | 0.65 | TCAaCtttcacTtcAGT |
| 17_69 | 56.83 | NA | 57.20 | 4.10 | TCAacTttcacTtcAGT |
| 17_70 | 76.86 | NA | 76.34 | 2.03 | TCaACtttcacTtcAGT |
| 17_71 | 63.44 | 10.55 | 64.68 | 5.87 | TCaAcTttcacTtcAGT |
| 17_72 | 62.56 | 5.79 | 61.72 | 1.34 | TcAAcTttcacTtcAGT |
| 17_73 | 60.51 | 6.25 | 67.89 | 3.45 | TCAACtttcacTtcaGT |
| 17_74 | 54.17 | NA | 56.84 | 3.66 | TCAActttcacTtcaGT |
| 17_75 | 66.76 | 4.71 | 62.81 | 3.26 | TCAaCTttcacTtcaGT |
| 17_76 | 66.23 | 5.60 | 53.07 | 13.10 | TCAaCtttcacTtcaGT |
| 17_77 | 59.39 | 8.21 | 63.25 | 4.95 | TCAacTttcacTtcaGT |
| 17_78 | 56.02 | 5.00 | 64.25 | 3.27 | TCaACtttcacTtcaGT |
| 17_79 | 45.91 | 4.00 | 56.13 | 3.45 | TCaAcTttcacTtcaGT |
| 17_80 | 69.86 | 6.08 | 69.85 | 3.93 | TCaaCtttcacTtcaGT |
| 17_81 | 65.32 | 5.73 | 70.58 | 4.02 | TCaacTttcacTtcaGT |
| 17_82 | 63.33 | 8.83 | 70.99 | 4.18 | TcAACtttcacTtcaGT |
| 17_83 | 68.96 | 8.36 | 74.25 | 5.87 | TcAaCtttcacTtcaGT |
| 17_84 | 63.62 | 7.64 | 81.25 | 4.70 | TcaaCtttcacTtcaGT |
| 17_85 | 83.30 | 4.59 | 84.25 | 2.62 | TCAACtttcactTCAGT |
| 17_86 | 37.09 | 7.98 | 43.15 | 2.13 | TCaActttcactTCAGT |
| 17_87 | 50.48 | 4.81 | 60.27 | 6.81 | TCaaCtttcactTCAGT |
| 17_88 | 53.38 | 5.35 | 56.84 | 5.09 | TCaacTttcactTCAGT |
| 17_89 | NA | NA | 43.67 | 3.84 | TCaactttcactTCAGT |
| 17_90 | 29.17 | 3.73 | 37.06 | 3.81 | TcAActttcactTCAGT |
| 17_91 | 61.71 | 7.15 | 71.61 | 3.90 | TcAaCtttcactTCAGT |
| 17_92 | 56.04 | 3.53 | 65.82 | 5.45 | TcaaCtttcactTCAGT |
| 17_93 | 45.09 | 4.71 | 56.40 | 2.59 | TcaactttcactTCAGT |
| 17_94 | 69.38 | 7.28 | 70.95 | 4.84 | TCAACtttcactTCaGT |
| 17_95 | 64.57 | 3.46 | 70.96 | 2.87 | TCAAcTttcactTCaGT |
| 17_96 | 34.51 | 2.38 | 39.62 | 1.63 | TCAActttcactTCaGT |
| 17_97 | 55.05 | 10.06 | 57.09 | 1.62 | TCAaCtttcactTCaGT |
| 17_98 | 64.97 | 7.46 | 63.11 | 2.12 | TCAacTttcactTCaGT |
| 17_99 | 36.70 | 4.12 | 39.75 | 1.43 | TCAactttcactTCaGT |
| 17_100 | 39.06 | NA | 41.61 | 1.24 | TCaActttcactTCaGT |
| 17_101 | 41.26 | 2.45 | 49.05 | 3.40 | TCaactttcactTCaGT |
| 17_102 | 78.96 | 10.63 | 60.35 | 2.12 | TcAACtttcactTCaGT |
| 17_103 | 32.50 | 2.83 | 36.44 | 1.34 | TcAActttcactTCaGT |
| 17_104 | 60.36 | 6.41 | 58.67 | 0.78 | TcAaCtttcactTCaGT |
| 17_105 | 58.78 | 3.01 | 65.37 | 2.47 | TcAacTttcactTCaGT |
| 17_106 | 41.78 | 7.71 | 45.57 | 2.93 | TcAactttcactTCaGT |
| 17_107 | 68.24 | 10.65 | 68.52 | 2.11 | TcaaCtttcactTCaGT |
| 17_108 | 63.66 | 6.15 | 69.87 | 1.49 | TcaactttcactTCaGT |
| 17_109 | 43.39 | 6.06 | 44.03 | 1.22 | TCAActttcactTcAGT |
| 17_110 | 67.71 | 3.99 | 68.24 | 2.49 | TCAaCtttcactTcAGT |
| 17_111 | 38.72 | 5.67 | 45.18 | 4.37 | TCaactttcactTcAGT |
| 17_112 | 74.81 | 8.54 | 82.12 | 2.07 | TcAACtttcactTcAGT |
| 17_113 | 45.61 | 3.48 | 49.46 | 3.00 | TcAActttcactTcAGT |
| 17_114 | 75.79 | 7.63 | 72.29 | 2.16 | TcAaCtttcactTcAGT |
| 17_115 | 75.42 | 15.41 | 74.41 | 3.07 | TcaActttcactTcAGT |
| 17_116 | 65.82 | 10.42 | 71.11 | 2.68 | TcaaCtttcactTcAGT |
| 17_117 | 59.41 | 10.07 | 62.29 | 5.94 | TcaactttcactTcAGT |
| 17_118 | 52.64 | NA | 52.72 | 2.61 | TCAACtttcactTcaGT |
| 17_119 | 39.63 | NA | 40.24 | 1.12 | TCAActttcactTcaGT |
| 17_120 | 59.98 | 2.92 | 50.20 | 0.85 | TCAaCtttcactTcaGT |
| 17_121 | 43.88 | 11.36 | 47.72 | 4.55 | TCAactttcactTcaGT |
| 17_122 | 64.88 | 13.05 | 60.50 | 3.00 | TCaaCtttcactTcaGT |
| 17_123 | 63.11 | 5.97 | 66.33 | 6.52 | TCaactttcactTcaGT |
| 17_124 | 56.82 | 7.60 | 52.41 | 2.44 | TcAaCtttcactTcaGT |
| 17_125 | 53.85 | 8.06 | 61.73 | 4.31 | TcAactttcactTcaGT |
| 17_126 | 81.50 | 15.86 | 84.13 | 4.80 | TcaActttcactTcaGT |
| 17_127 | 78.91 | 10.65 | 82.69 | 2.51 | TcaactttcactTcaGT |
| 17_128 | 81.11 | 11.24 | 78.80 | 1.05 | TCAACtttcacttCAGT |
| 17_129 | 32.28 | 2.57 | 39.12 | 1.07 | TCAactttcacttCAGT |
| 17_130 | 70.27 | 8.13 | 72.06 | 1.44 | TCaACtttcacttCAGT |
| 17_131 | 52.53 | 5.34 | 51.48 | 1.51 | TCaAcTttcacttCAGT |
| 17_132 | 39.54 | 5.34 | 40.49 | 2.90 | TCaActttcacttCAGT |
| 17_133 | 49.75 | 8.73 | 51.25 | 2.19 | TCaaCtttcacttCAGT |
| 17_134 | 40.11 | 4.72 | 46.40 | 3.25 | TCaacTttcacttCAGT |
| 17_135 | 32.68 | 5.78 | 44.12 | 1.28 | TCaactttcacttCAGT |
| 17_136 | 73.83 | 11.05 | 64.31 | 14.71 | TcAACtttcacttCAGT |
| 17_137 | 27.45 | 3.58 | 37.37 | 0.87 | TcAActttcacttCAGT |
| 17_138 | 52.94 | 2.36 | 52.33 | 6.75 | TcAaCtttcacttCAGT |
| 17_139 | 33.04 | 3.96 | 41.18 | 2.84 | TcAactttcacttCAGT |
| 17_140 | 51.65 | 1.57 | 52.29 | 3.62 | TCAAcTttcacttCaGT |
| 17_141 | 61.72 | 2.80 | 58.93 | 0.97 | TCAaCTttcacttCaGT |
| 17_142 | 46.19 | NA | 52.83 | 5.45 | TCAaCtttcacttCaGT |
| 17_143 | 43.84 | 1.08 | 45.66 | 0.98 | TCAacTttcacttCaGT |
| 17_144 | 37.39 | 2.38 | 43.74 | 1.32 | TCAactttcacttCaGT |
| 17_145 | 67.26 | 7.35 | 74.40 | 4.87 | TCaACTttcacttCaGT |
| 17_146 | 56.45 | 2.94 | 56.68 | 0.48 | TCaACtttcacttCaGT |
| 17_147 | 47.22 | 1.68 | 54.43 | 1.21 | TCaAcTttcacttCaGT |
| 17_148 | 43.18 | 2.71 | 56.05 | 1.42 | TCaaCTttcacttCaGT |
| 17_149 | 45.97 | NA | 53.84 | 3.68 | TCaacTttcacttCaGT |
| 17_150 | 59.24 | 6.22 | 60.59 | 3.40 | TcAACtttcacttCaGT |
| 17_151 | 51.93 | NA | 61.55 | 5.08 | TcAaCtttcacttCaGT |
| 17_152 | 47.41 | 5.67 | 52.89 | 3.10 | TcAactttcacttCaGT |
| 17_153 | 65.27 | 4.09 | 69.29 | 7.55 | TcaActttcacttCaGT |
| 17_154 | 53.74 | NA | 62.46 | 1.61 | TcaaCTttcacttCaGT |
| 17_155 | 66.62 | 5.23 | 74.14 | 3.90 | TcaactttcacttCaGT |
| 17_156 | 48.09 | 0.70 | 49.14 | 1.49 | TCAAcTttcacttcAGT |
| 17_157 | 38.49 | 2.92 | 43.72 | 1.30 | TCAActttcacttcAGT |
| 17_158 | 59.33 | 3.81 | 63.90 | 1.94 | TCAaCTttcacttcAGT |
| 17_159 | 56.79 | 9.47 | 55.56 | 2.69 | TCAaCtttcacttcAGT |
| 17_160 | 50.32 | 7.20 | 48.93 | 2.20 | TCaaCTttcacttcAGT |
| 17_161 | 40.36 | 4.00 | 45.81 | 1.30 | TCaacTttcacttcAGT |
| 17_162 | 64.11 | 4.76 | 62.08 | 1.69 | TcAaCtttcacttcAGT |
| 17_163 | 58.28 | NA | 59.97 | 2.18 | TcAactttcacttcAGT |
| 17_164 | 76.29 | 13.13 | 77.15 | 3.83 | TcaActttcacttcAGT |
| 17_165 | 78.09 | 15.89 | 72.59 | 8.69 | TcaactttcacttcAGT |
| 17_166 | 62.49 | 3.63 | 64.37 | 5.16 | TCAACTttcacttcaGT |
| 17_167 | 50.03 | 8.03 | 54.73 | 1.30 | TCAACtttcacttcaGT |
| 17_168 | 51.60 | 9.81 | 52.08 | 4.48 | TCAAcTttcacttcaGT |
| 17_169 | 46.17 | 5.15 | 51.40 | 2.49 | TCAActttcacttcaGT |
| 17_170 | 52.75 | 11.01 | 54.83 | 2.69 | TCAaCTttcacttcaGT |
| 17_171 | 53.33 | 9.21 | 54.36 | 2.78 | TCAaCtttcacttcaGT |
| 17_172 | 58.21 | 6.31 | 58.05 | 1.23 | TCAactttcacttcaGT |
| 17_173 | 53.76 | 2.90 | 58.61 | 1.13 | TCaACTttcacttcaGT |
| 17_174 | 50.25 | 5.79 | 50.99 | 7.67 | TCaACtttcacttcaGT |
| 17_175 | 51.82 | 4.61 | 54.72 | 1.85 | TCaActttcacttcaGT |
| 17_176 | 53.43 | NA | 58.36 | 6.34 | TCaaCtttcacttcaGT |
| 17_177 | 57.85 | 3.78 | 63.73 | 2.53 | TCaacTttcacttcaGT |
| 17_178 | 62.40 | 7.11 | 60.69 | 2.19 | TcAActttcacttcaGT |
| 17_179 | 58.09 | 9.19 | 57.23 | 4.50 | TcAaCtttcacttcaGT |
| 17_180 | 74.45 | 11.02 | 75.46 | 4.00 | TcAactttcacttcaGT |
| 17_181 | 90.80 | 14.30 | 82.83 | 2.65 | TcaActttcacttcaGT |
| 17_182 | 74.91 | NA | 75.31 | 4.39 | TcaaCtttcacttcaGT |
| 17_183 | 88.59 | 4.23 | 85.23 | 2.44 | TcaactttcacttcaGT |
| 18_1 | 32.92 | 3.39 | 35.69 | 3.82 | TCAactttcacttCAG |
| 18_250 | 100.08 | 10.66 | 88.51 | 4.20 | TCAACtttcaCTTCAG |
| 18_251 | 84.40 | 7.39 | 80.86 | 4.12 | TCAActttcaCTTCAG |
| 18_252 | 91.54 | 3.68 | 89.30 | 5.79 | TCAaCtttcaCTTCAG |
| 18_253 | 91.81 | 6.31 | 89.37 | 3.90 | TCaACtttcaCTTCAG |
| 18_254 | 85.25 | 10.05 | 84.67 | 2.91 | TCaaCtttcaCTTCAG |
| 18_255 | 86.24 | 2.27 | 87.98 | 0.91 | TcaaCtttcaCTTCAG |
| 18_256 | 78.51 | 4.22 | 82.48 | 9.24 | TcaactttcaCTTCAG |
| 18_257 | 89.59 | 11.37 | 90.01 | 5.75 | TcAaCtttcaCTTcAG |
| 18_258 | 95.95 | 14.37 | 92.27 | 12.06 | TcaaCtttcaCTTcAG |
| 18_259 | 81.62 | 8.01 | 75.93 | 5.23 | TcaactttcaCTTcAG |
| 18_260 | 89.34 | 4.48 | 92.90 | 6.69 | TCAaCtttcaCTtCAG |
| 18_261 | 54.74 | NA | 59.78 | 4.39 | TCAactttcaCTtCAG |
| 18_262 | 91.32 | 12.46 | 85.83 | 4.88 | TCaaCtttcaCTtCAG |
| 18_263 | 53.49 | 6.41 | 55.73 | 1.72 | TCaactttcaCTtCAG |
| 18_264 | 77.00 | 7.13 | 83.85 | 2.44 | TcAACtttcaCTtCAG |
| 18_265 | 82.71 | 2.41 | 80.20 | 3.21 | TcaaCtttcaCTtCAG |
| 18_266 | 65.50 | 14.42 | 63.32 | 7.76 | TcaactttcaCTtCAG |
| 18_267 | 88.30 | 14.79 | 88.12 | 2.67 | TCAACtttcaCTtcAG |
| 18_268 | 85.83 | 5.66 | 80.25 | 1.37 | TCAActttcaCTtcAG |
| 18_269 | 84.52 | 3.17 | 89.90 | 6.04 | TCAaCtttcaCTtcAG |
| 18_270 | 57.28 | 7.24 | 62.34 | NA | TCAactttcaCTtcAG |
| 18_271 | 84.49 | 8.06 | 91.51 | 3.02 | TCaACtttcaCTtcAG |
| 18_272 | 76.13 | 4.46 | 79.90 | NA | TCaActttcaCTtcAG |
| 18_273 | 85.88 | 7.38 | 97.42 | 4.00 | TcAaCtttcaCTtcAG |
| 18_274 | 95.40 | 13.18 | 95.86 | 1.55 | TcaaCtttcaCTtcAG |
| 18_275 | 95.60 | 10.21 | 92.33 | 2.77 | TCAACtttcaCtTCAG |
| 18_276 | 83.72 | 6.59 | 80.77 | 2.02 | TCAActttcaCtTCAG |
| 18_277 | 90.13 | 10.30 | 96.27 | 13.83 | TCAaCtttcaCtTCAG |
| 18_278 | 55.67 | 8.13 | 62.46 | 6.54 | TCAactttcaCtTCAG |
| 18_279 | 87.22 | 13.33 | 88.16 | 8.73 | TCaACtttcaCtTCAG |
| 18_280 | 76.65 | 3.97 | 79.84 | 12.72 | TCaActttcaCtTCAG |
| 18_281 | 81.18 | 8.97 | 84.87 | 7.12 | TCaaCtttcaCtTCAG |
| 18_282 | 61.04 | 7.74 | 61.76 | 1.66 | TCaactttcaCtTCAG |
| 18_283 | 84.65 | 3.34 | 80.88 | 2.96 | TCaaCtttcaCtTcAG |
| 18_284 | 61.02 | 6.86 | 62.10 | 2.82 | TCaactttcaCtTcAG |
| 18_285 | 86.61 | 3.69 | 95.03 | 18.61 | TcAACtttcaCtTcAG |
| 18_286 | 84.98 | 9.65 | 85.00 | 14.32 | TcAActttcaCtTcAG |
| 18_287 | 86.45 | 4.35 | 88.69 | 7.72 | TcAaCtttcaCtTcAG |
| 18_288 | 57.67 | 1.82 | 61.38 | NA | TcAactttcaCtTcAG |
| 18_289 | 79.05 | 6.07 | 83.92 | 4.10 | TcaActttcaCtTcAG |
| 18_290 | 87.52 | 9.96 | 91.14 | 2.20 | TcaaCtttcaCtTcAG |
| 18_291 | 73.29 | 5.03 | 69.25 | 5.43 | TcaactttcaCtTcAG |
| 18_292 | 72.78 | 7.03 | 68.16 | 1.00 | TCAACtttcaCttCAG |
| 18_293 | 59.43 | 5.50 | 58.08 | 2.89 | TCAActttcaCttCAG |
| 18_294 | 75.84 | 3.56 | 63.66 | 3.73 | TCAaCtttcaCttCAG |
| 18_295 | 46.89 | 3.57 | 49.06 | 2.63 | TCAactttcaCttCAG |
| 18_296 | 65.42 | 3.75 | 63.31 | 3.08 | TCaACtttcaCttCAG |
| 18_297 | 58.20 | 6.79 | 55.76 | 1.22 | TCaActttcaCttCAG |
| 18_298 | 66.88 | 4.87 | 66.09 | 3.03 | TCaaCtttcaCttCAG |
| 18_299 | 57.00 | 3.54 | 52.43 | 0.96 | TCaactttcaCttCAG |
| 18_300 | 67.40 | 4.43 | 64.15 | 3.50 | TcAACtttcaCttCAG |
| 18_301 | 76.29 | 2.94 | 66.61 | 0.93 | TcaACtttcaCttCAG |
| 18_302 | 79.40 | 6.94 | 75.09 | 2.40 | TcaActttcaCttCAG |
| 18_303 | 80.86 | 2.61 | 67.53 | 3.70 | TCAACtttcaCttcAG |
| 18_304 | 67.19 | 3.65 | 64.77 | 2.65 | TCAActttcaCttcAG |
| 18_305 | 79.81 | 7.90 | 76.61 | 4.75 | TCAaCtttcaCttcAG |
| 18_306 | 65.48 | 4.30 | 60.08 | 1.89 | TCAactttcaCttcAG |
| 18_307 | 70.08 | 6.13 | 70.40 | 2.08 | TCaACtttcaCttcAG |
| 18_308 | 70.99 | 2.21 | 71.46 | 3.87 | TCaActttcaCttcAG |
| 18_309 | 69.43 | 6.30 | 81.14 | 12.38 | TCaaCtttcaCttcAG |
| 18_310 | 73.04 | 7.86 | 73.31 | 4.69 | TCaactttcaCttcAG |
| 18_311 | 72.32 | 9.45 | 78.61 | 8.91 | TcAACtttcaCttcAG |
| 18_312 | 67.82 | 11.23 | 78.05 | 7.27 | TcAActttcaCttcAG |
| 18_313 | 75.81 | 10.76 | 78.01 | 7.76 | TcAaCtttcaCttcAG |
| 18_314 | 66.04 | 5.65 | 75.33 | 8.56 | TcAactttcaCttcAG |
| 18_315 | 78.82 | 5.66 | 75.34 | 2.78 | TcaACtttcaCttcAG |
| 18_316 | 87.37 | 14.72 | 95.41 | 6.94 | TcaaCtttcaCttcAG |
| 18_317 | 79.19 | 4.27 | 94.13 | 12.76 | TcaactttcaCttcAG |
| 18_318 | 59.57 | 10.72 | 63.41 | 2.62 | TCAActttcacTTCAG |
| 18_319 | 84.55 | 4.72 | 81.60 | 3.53 | TCAaCtttcacTTCAG |
| 18_320 | 72.74 | 2.03 | 79.32 | 10.24 | TCaACtttcacTTCAG |
| 18_321 | 72.73 | 6.17 | 74.90 | 3.78 | TcAACtttcacTTCAG |
| 18_322 | 70.71 | 12.19 | 72.65 | 3.47 | TcAaCtttcacTTCAG |
| 18_323 | 63.05 | 4.68 | 64.11 | 2.23 | TcaaCtttcacTTCAG |
| 18_324 | 90.00 | 7.49 | 79.94 | 4.07 | TCAACtttcacTTcAG |
| 18_325 | 79.21 | 4.73 | 75.34 | 2.42 | TCAaCtttcacTTcAG |
| 18_326 | 68.92 | NA | 67.74 | 4.83 | TCaaCtttcacTTcAG |
| 18_327 | 56.44 | 4.90 | 56.48 | 2.86 | TcAActttcacTTcAG |
| 18_328 | 75.87 | 4.14 | 71.99 | 4.42 | TcAaCtttcacTTcAG |
| 18_329 | 61.35 | 2.64 | 57.83 | 2.46 | TcAactttcacTTcAG |
| 18_330 | 82.34 | 3.56 | 78.64 | 4.39 | TcaaCtttcacTTcAG |
| 18_331 | 75.40 | 6.43 | 72.02 | 3.95 | TcaactttcacTTcAG |
| 18_332 | 72.69 | 7.00 | 73.99 | 3.23 | TCAaCtttcacTtCAG |
| 18_333 | 47.08 | 4.26 | 45.64 | 2.17 | TCaActttcacTtCAG |
| 18_334 | 63.55 | 2.17 | 61.47 | 5.18 | TCaaCtttcacTtCAG |
| 18_335 | 45.43 | 2.17 | 43.67 | 0.51 | TcAActttcacTtCAG |
| 18_336 | 62.16 | 1.68 | 63.10 | 4.22 | TcaactttcacTtCAG |
| 18_337 | 68.12 | 1.83 | 69.62 | 5.48 | TCAACtttcacTtcAG |
| 18_338 | 58.66 | 3.79 | 55.57 | 3.90 | TCAActttcacTtcAG |
| 18_339 | 64.78 | 3.20 | 67.31 | 4.73 | TCAaCtttcacTtcAG |
| 18_340 | 73.84 | 12.62 | 70.76 | 2.66 | TCaaCtttcacTtcAG |
| 18_341 | 63.86 | 1.31 | 62.80 | 2.97 | TCaactttcacTtcAG |
| 18_342 | 63.62 | 7.33 | 62.67 | 3.14 | TcAACtttcacTtcAG |
| 18_343 | 77.34 | 8.12 | 76.95 | 8.74 | TcAaCtttcacTtcAG |
| 18_344 | 77.52 | 4.63 | 72.61 | 19.40 | TcaaCtttcacTtcAG |
| 18_345 | 44.88 | 5.16 | 44.48 | 2.03 | TCaACtttcactTCAG |
| 18_346 | 33.58 | 3.96 | 33.46 | 0.75 | TCaActttcactTCAG |
| 18_347 | 25.34 | 3.90 | 27.48 | 1.20 | TcAActttcactTCAG |
| 18_348 | 72.22 | 13.10 | 69.54 | 2.55 | TcaActttcactTCAG |
| 18_349 | 60.34 | 3.62 | 62.20 | 3.43 | TcaactttcactTCAG |
| 18_350 | 42.64 | 7.75 | 39.08 | 1.64 | TCAActttcactTcAG |
| 18_351 | 64.87 | 4.90 | 60.46 | 2.58 | TCaaCtttcactTcAG |
| 18_352 | 60.50 | 8.75 | 58.85 | NA | TCaactttcactTcAG |
| 18_353 | 46.91 | 7.66 | 48.41 | 2.35 | TcAActttcactTcAG |
| 18_354 | 56.92 | 5.54 | 55.90 | 3.41 | TcaACtttcactTcAG |
| 18_355 | 83.71 | 14.79 | 81.27 | 2.26 | TcaActttcactTcAG |
| 18_356 | 39.74 | 8.56 | 46.46 | NA | TCaACtttcacttCAG |
| 18_357 | 38.75 | 4.00 | 38.86 | 1.61 | TCaActttcacttCAG |
| 18_358 | 38.88 | 4.61 | 43.88 | 5.77 | TcaACtttcacttCAG |
| 18_359 | 77.53 | 8.61 | 72.87 | 3.73 | TcaActttcacttCAG |
| 18_360 | 78.21 | NA | 75.73 | 4.38 | TcaactttcacttCAG |
| 18_361 | 57.41 | NA | 51.70 | 2.51 | TcAaCtttcacttcAG |
| 19_4 | 101.90 | 8.84 | 105.29 | 4.25 | TGTTTcaataCTAAAA |
| 19_5 | 105.24 | 11.89 | 100.23 | 3.22 | TGTTtcaataCTAAAA |
| 19_6 | 99.75 | 6.33 | 104.03 | 3.46 | TGTtTcaataCTAAAA |
| 19_7 | 91.29 | NA | 91.20 | 2.56 | TGTttcaataCTAAAA |
| 19_8 | 106.37 | NA | 100.46 | 3.70 | TGtTTcaataCTAAAA |
| 19_9 | 108.42 | 11.96 | 101.59 | 4.05 | TGttTcaataCTAAAA |
| 19_10 | 100.39 | 8.50 | 102.93 | 6.06 | TgTTTcaataCTAAAA |
| 19_11 | 90.83 | 3.68 | 92.38 | 3.27 | TGTTTcaataCTAaAA |
| 19_12 | 90.86 | 3.89 | 91.69 | 3.53 | TGTTtcaataCTAaAA |
| 19_13 | 89.85 | 3.87 | 91.34 | 2.59 | TGTtTcaataCTAaAA |
| 19_14 | 94.01 | 8.75 | 94.66 | 2.33 | TGTttcaataCTAaAA |
| 19_15 | 92.12 | 2.54 | 91.25 | 2.22 | TGtTTcaataCTAaAA |
| 19_16 | 97.86 | 5.30 | 93.85 | 1.92 | TgTTTcaataCTAaAA |
| 19_17 | 105.50 | 15.59 | 99.75 | 4.80 | TGTTTcaataCTaAAA |
| 19_18 | 102.61 | 5.30 | 96.26 | 2.40 | TGTTtcaataCTaAAA |
| 19_19 | 94.76 | 5.45 | 94.05 | 2.41 | TGTtTcaataCTaAAA |
| 19_20 | 97.80 | 9.88 | 102.61 | 9.09 | TGTTTcaataCTaaAA |
| 19_21 | 95.95 | 9.14 | 89.84 | 2.06 | TGTTtcaataCTaaAA |
| 19_22 | 101.79 | 7.29 | 95.45 | 3.90 | TGTTTcaataCtAAAA |

From these data it can be seen that the LNA-gapmer designs based on the motif sequence with SEQ ID NO: 19 have very low (between 0 and 10%) PAPD5 and PAPD7 knock down.

### Example 4: In Vitro EC50 and Efficacy of Selected Antisense Oligonucleotides in HeLa Cells

The EC50 and efficacy (KID) of the best performing oligonucleotides from Example 1 and 3 was determined using the same assay with the following oligonucleotide concentrations 50, 15.81, 5.00, 1.58, 0.50, 0.16, 0.05, and 0.016 μM.

EC 50, max KD (efficacy) of the PAPD5 and PAPD7 mRNA expression was calculated using the R-function drm( ) from the drc package (v3.0-1) a four-parameter log-logistic function is fitted to the expression of the gene of interest as a function of oligonucleotide concentration to obtain a value for EC50 and maximum knock-down. The results are shown in Table 16.

TABLE 16
EC50 and Max KD of anti-PAPD5/PAPD7 compounds on PAPD5 and PAPD7
mRNA expression in HeLa cells.

| | PAPD5 | | | | PAPD7 | | | | |
|---|---|---|---|---|---|---|---|---|---|
| | Max KD | | EC50 | | Max KD | | EC50 | | |
| CMP | % of saline | | μM | | % of saline | | μM | | |
| ID NO | Avg | sd | Avg | sd | Avg | sd | Avg | sd | Compound |
| 17_7 | 1.45 | 7.29 | 2.40 | 0.55 | 8.00 | 6.58 | 3.13 | 0.65 | TcAactttcactTcAGT |
| 17_8 | 7.66 | 4.14 | 3.08 | 0.42 | 5.37 | 5.16 | 4.00 | 0.62 | TcAActttcactTcaGT |
| 17_10 | 0.00 | 2.40 | 2.30 | 0.19 | 3.31 | 5.90 | 3.79 | 0.68 | TCAActttcacttCaGT |
| 17_12 | 6.52 | 3.37 | 2.72 | 0.31 | 11.14 | 4.37 | 3.32 | 0.49 | TCaactttcacttCaGT |
| 17_13 | 0.68 | 5.12 | 2.43 | 0.42 | 2.29 | 4.83 | 3.64 | 0.55 | TcAActttcacttCaGT |
| 17_14 | 0.19 | 5.00 | 2.51 | 0.42 | 3.13 | 4.54 | 3.69 | 0.52 | TCAactttcacttcAGT |
| 17_51 | 3.29 | 3.89 | 1.41 | 0.21 | 5.81 | 1.20 | 1.78 | 0.08 | TCaactttcacTtCAGT |
| 17_57 | 2.61 | 7.96 | 1.54 | 0.47 | 3.07 | 3.45 | 1.76 | 0.21 | TCAactttcacTtCaGT |
| 17_86 | 0.00 | 3.77 | 1.19 | 0.17 | 0.00 | 3.32 | 2.01 | 0.22 | TCaActttcactTCAGT |
| 17_89 | 6.03 | 2.64 | 1.02 | 0.11 | 9.23 | 3.65 | 1.44 | 0.21 | TCaactttcactTCAGT |
| 17_90 | 2.43 | 5.44 | 1.38 | 0.29 | 1.87 | 5.63 | 1.95 | 0.40 | TcAActttcactTCAGT |
| 17_96 | 3.27 | 2.62 | 1.85 | 0.18 | 0.00 | 3.44 | 1.99 | 0.24 | TCAActttcactTCaGT |
| 17_99 | 0.00 | 3.61 | 1.42 | 0.18 | 0.55 | 5.03 | 1.57 | 0.28 | TCAactttcactTCaGT |
| 17_100 | 1.01 | 2.65 | 1.66 | 0.16 | 3.81 | 3.46 | 1.89 | 0.24 | TCaActttcactTCaGT |
| 17_103 | 0.00 | 2.69 | 1.09 | 0.12 | 0.00 | 3.70 | 1.46 | 0.21 | TcAActttcactTCaGT |
| 17_111 | 3.45 | 3.62 | 1.39 | 0.20 | 2.65 | 5.82 | 2.03 | 0.41 | TCaactttcactTcAGT |
| 17_119 | 0.00 | 6.24 | 1.75 | 0.39 | 0.30 | 3.81 | 1.86 | 0.25 | TCAActttcactTcaGT |
| 17_129 | 0.00 | 2.62 | 1.02 | 0.11 | 2.60 | 2.44 | 1.41 | 0.13 | TCAactttcacttCAGT |
| 17_132 | 1.71 | 2.02 | 1.27 | 0.10 | 0.00 | 4.17 | 1.74 | 0.26 | TCaActttcacttCAGT |
| 17_135 | 0.00 | 3.23 | 1.24 | 0.14 | 8.56 | 4.86 | 2.04 | 0.38 | TCaactttcacttCAGT |
| 17_137 | 0.00 | 2.80 | 1.07 | 0.12 | 1.34 | 3.94 | 1.64 | 0.23 | TcAActttcacttCAGT |
| 17_139 | 0.00 | 3.62 | 1.43 | 0.20 | 2.48 | 5.82 | 1.89 | 0.39 | TcAactttcacttCAGT |
| 17_144 | 0.91 | 2.35 | 1.40 | 0.12 | 1.53 | 1.58 | 1.95 | 0.11 | TCAactttcacttCaGT |
| 17_157 | 2.94 | 2.87 | 1.27 | 0.14 | 2.32 | 3.12 | 1.62 | 0.18 | TCAActttcacttcAGT |
| 18_1 | 2.74 | 1.41 | 1.82 | 0.09 | 5.06 | 2.24 | 2.03 | 0.16 | TCAactttcacttCAG |
| 18_5 | 4.25 | 6.93 | 4.08 | 0.82 | 6.91 | 4.42 | 3.35 | 0.47 | TCAActttcacTtCAG |
| 18_6 | 5.49 | 4.00 | 2.97 | 0.39 | 8.16 | 4.67 | 2.93 | 0.45 | TCaactttcacTtCAG |
| 18_10 | 0.00 | 6.55 | 1.60 | 0.38 | 0.00 | 3.59 | 2.17 | 0.26 | TCAActttcactTCAG |
| 18_12 | 1.34 | 3.34 | 1.69 | 0.20 | 0.84 | 4.01 | 2.37 | 0.32 | TCAactttcactTCAG |
| 18_15 | 5.89 | 2.84 | 2.92 | 0.28 | 6.85 | 3.64 | 3.10 | 0.39 | TcAACtttcactTcAG |
| 18_18 | 4.23 | 4.44 | 2.71 | 0.41 | 2.40 | 10.93 | 2.76 | 0.88 | TCAACtttcacttCAG |
| 18_19 | 2.22 | 3.25 | 2.04 | 0.23 | 1.66 | 5.12 | 2.53 | 0.44 | TCAActttcacttCAG |
| 18_20 | 0.00 | 3.21 | 2.56 | 0.27 | 0.00 | 4.96 | 2.81 | 0.47 | TCAaCtttcacttCAG |
| 18_21 | 2.13 | 3.08 | 2.52 | 0.25 | 5.72 | 2.45 | 2.73 | 0.23 | TCaaCtttcacttCAG |
| 18_23 | 0.49 | 4.56 | 2.65 | 0.39 | 0.53 | 3.28 | 3.02 | 0.31 | TCAACtttcacttcAG |
| 18_24 | 0.29 | 6.14 | 2.82 | 0.54 | 0.00 | 6.27 | 2.95 | 0.61 | TCAActttcacttcAG |
| 18_25 | 2.22 | 5.75 | 2.55 | 0.49 | 0.00 | 3.68 | 3.13 | 0.36 | TCAaCtttcacttcAG |
| 18_27 | 0.00 | 4.13 | 2.30 | 0.30 | 1.21 | 2.04 | 2.87 | 0.19 | TCaACtttcacttcAG |
| 18_28 | 10.11 | 3.82 | 4.52 | 0.56 | 12.26 | 11.67 | 5.13 | 1.78 | TCaaCtttcacttcAG |
| 18_30 | 1.60 | 3.21 | 2.56 | 0.27 | 0.00 | 3.47 | 3.10 | 0.34 | TcAACtttcacttcAG |
| 18_346 | 0.56 | 3.27 | 1.27 | 0.17 | 1.43 | 1.58 | 1.49 | 0.09 | TCaActttcactTCAG |
| 18_347 | 0.16 | 3.81 | 0.87 | 0.14 | 0.00 | 1.55 | 1.17 | 0.07 | TcAActttcactTCAG |
| 18_350 | 0.00 | 3.12 | 1.54 | 0.17 | 1.43 | 1.29 | 2.10 | 0.09 | TCAActttcactTcAG |
| 18_357 | 0.00 | 2.87 | 1.61 | 0.18 | 0.00 | 1.97 | 2.18 | 0.15 | TCaActttcacttCAG |
| 18_358 | 0.00 | 2.30 | 1.54 | 0.13 | 0.15 | 1.91 | 2.31 | 0.14 | TcaACtttcacttCAG |

### Example 5: In Vitro Effect on HBV Infected ASGPR-dHepaRG Cells Using Selected Antisense Oligonucleotides Targeting PAPD5 and PAPD7

A selection of the oligonucleotides screened in example 3 was screened in ASGPR-dHepaRG essentially using the assay of example 2 with the following changes. The screening was conducted in HBV infected ASGPR-dHepaRG at the following concentrations 20, 6.67 and 2.22 μM of oligonucleotide and with the comparative molecules in table 17.

For comparative purposes combinations of a single targeting PAPD7 and a single targeting PAPD7 oligonucleotide in table 17 were tested together with the oligonucleotides of the invention.

TABLE 17
Combination of single targeting PAPD5 and PAPD7
oligonucleotide

| | | SEQ ID | |
|---|---|---|---|
| Description | Compound | NO | Reference |
| PAPD5 and | CAAaggttgttgtacTCT | 31 | PCT/EP2017/ |
| PAPD7 single | | | 064980 |
| targeting | CAGTtttatgctaatCA | 32 | PCT/EP2017/ |
| combination 1 | | | 064980 |
| (combo1) | | | |
| PAPD5 and | GTAttcttattcttgCT | 33 | PCT/EP2017/ |
| PAPD7 single | | | 064980 |
| targeting | CATTgcttttataatccTA | 34 | PCT/EP2017/ |
| combination 2 | | | 064980 |
| (combo2) | | | |

The reduction of HBsAg and HBeAg levels are shown in table 18 and 19, the larger the value the larger the inhibition.

TABLE 18
in vitro efficacy on HBsAg of anti-PAPD5/PAPD7 compounds in
three concentrations (average of 3) in HBV infected ASGPR-
dHepaRG cells.

| CMP ID | 20 μM | | 6.67 μM | | 2.22 μM | | |
|---|---|---|---|---|---|---|---|
| NO | Avg | sd | Avg | sd | Avg | sd | Compound |
| 17_51 | −9.61 | 19.93 | −30.60 | 9.19 | −33.16 | 6.96 | TCaactttcacTtCAGT |
| 17_57 | 9.44 | 6.27 | −18.18 | 8.10 | −33.24 | 6.19 | TCAactttcacTtCaGT |
| 17_86 | 20.58 | 5.80 | −5.34 | 4.43 | −8.03 | 5.54 | TCaActttcactTCAGT |
| 17_89 | 2.66 | 3.48 | −12.71 | 2.14 | −7.18 | 7.05 | TCaactttcactTCAGT |
| 17_90 | 40.07 | 6.93 | 3.05 | 14.90 | −11.67 | 7.22 | TcAActttcactTCAGT |
| 17_96 | 58.09 | 7.77 | 36.82 | 3.53 | 4.92 | 4.06 | TCAActttcactTCaGT |
| 17_99 | 25.54 | 6.97 | 5.75 | 8.72 | −7.25 | 5.93 | TCAactttcactTCaGT |
| 17_100 | 43.85 | 7.30 | 15.20 | 12.19 | −10.24 | 9.46 | TCaActttcactTCaGT |
| 17_103 | 41.44 | 9.31 | 25.07 | 2.93 | 9.98 | 3.98 | TcAActttcactTCaGT |
| 17_111 | −5.59 | 7.25 | −7.04 | 3.62 | −8.11 | 6.03 | TCaactttcactTcAGT |
| 17_119 | 73.06 | 2.91 | 51.21 | 3.44 | 13.11 | 9.33 | TCAActttcactTcaGT |
| 17_129 | 37.17 | 10.95 | 9.73 | 10.63 | 2.19 | 14.92 | TCAactttcacttCAGT |
| 17_132 | 41.31 | 5.57 | 11.54 | 5.29 | −10.07 | 4.00 | TCaActttcacttCAGT |
| 17_135 | 3.24 | 6.43 | 2.61 | 10.50 | −13.05 | 2.27 | TCaactttcacttCAGT |
| 17_137 | 60.37 | 4.60 | 44.00 | 4.51 | 13.77 | 1.76 | TcAActttcacttCAGT |
| 17_139 | 51.89 | 6.99 | 25.28 | 5.62 | −9.98 | 3.81 | TcAactttcacttCAGT |
| 17_144 | 15.51 | 9.49 | 2.98 | 11.13 | −14.47 | 6.57 | TCAactttcacttCaGT |
| 17_157 | 60.44 | 2.21 | 43.72 | 7.14 | −0.43 | 5.64 | TCAActttcacttcAGT |
| 18_1 | 90.68 | 1.23 | 75.99 | 2.96 | 17.58 | 8.44 | TCAactttcacttCAG |
| 18_346 | 87.27 | 1.42 | 51.65 | 5.99 | −0.36 | 6.52 | TCaActttcactTCAG |
| 18_347 | 88.09 | 2.70 | 66.31 | 4.12 | 1.27 | 11.46 | TcAActttcactTCAG |
| 18_350 | 82.82 | 2.94 | 68.17 | 3.68 | 25.39 | 3.40 | TCAActttcactTcAG |
| 18_357 | 91.46 | 1.63 | 77.08 | 2.24 | 35.54 | 3.18 | TCaActttcacttCAG |
| 18_358 | 83.98 | 3.39 | 63.78 | 6.55 | 26.29 | 5.45 | TcaACtttcacttCAG |
| Combo1 | 72.08 | 0.75 | 58.03 | 2.25 | 21.27 | 8.25 | |
| Combo2 | 71.77 | 4.54 | 67.54 | 3.72 | 50.53 | 5.82 | |

TABLE 19
in vitro efficacy on HBeAg of anti-PAPD5/PAPD7 compounds
in three concentrations (average of 3) in HBV infected
ASGPR-dHepaRG cells.

| CMP | 20 μM | | 6.67 μM | | 2.22 μM | | |
|---|---|---|---|---|---|---|---|
| ID NO | Avg | sd | Avg | sd | Avg | sd | Compound |
| 17_51 | −39.37 | 39.73 | −71.52 | 24.98 | −89.89 | 24.95 | TCaactttcacTtCAGT |
| 17_57 | 2.88 | 4.42 | −38.92 | 11.07 | −76.67 | 6.90 | TCAactttcacTtCaGT |
| 17_86 | 22.69 | 5.54 | −20.63 | 5.70 | −42.45 | 4.40 | TCaActttcactTCAGT |
| 17_89 | −11.41 | 3.45 | −36.53 | 9.77 | −34.92 | 9.69 | TCaactttcactTCAGT |
| 17_90 | 50.40 | 8.09 | −4.45 | 25.09 | −36.73 | 16.16 | TcAActttcactTCAGT |
| 17_96 | 68.32 | 9.42 | 47.89 | 5.53 | 2.93 | 16.50 | TCAActttcactTCaGT |
| 17_99 | 34.82 | 8.81 | 15.96 | 21.39 | −13.36 | 13.51 | TCAactttcactTCaGT |
| 17_100 | 55.17 | 5.99 | 20.03 | 20.34 | −25.12 | 18.75 | TCaActttcactTCaGT |
| 17_103 | 48.08 | 14.67 | 28.80 | 9.35 | 7.18 | 12.00 | TcAActttcactTCaGT |
| 17_111 | −5.24 | 15.62 | −10.26 | 3.22 | −18.78 | 9.24 | TCaactttcactTcAGT |
| 17_119 | 83.29 | 3.11 | 69.67 | 1.75 | 24.17 | 9.29 | TCAActttcactTcaGT |
| 17_129 | 47.32 | 8.81 | 19.21 | 17.51 | −6.65 | 24.28 | TCAactttcacttCAGT |
| 17_132 | 59.04 | 4.63 | 21.83 | 1.86 | −14.91 | 0.44 | TCaActttcacttCAGT |
| 17_135 | 8.35 | 11.28 | 2.09 | 13.51 | −25.60 | 9.12 | TCaactttcacttCAGT |
| 17_137 | 73.77 | 2.83 | 58.40 | 3.45 | 18.22 | 1.27 | TcAActttcacttCAGT |
| 17_139 | 64.19 | 7.67 | 39.45 | 5.57 | −17.73 | 3.08 | TcAactttcacttCAGT |
| 17_144 | 24.74 | 7.77 | 12.21 | 16.40 | −31.19 | 11.36 | TCAactttcacttCaGT |
| 17_157 | 75.79 | 1.10 | 61.26 | 4.35 | 9.64 | 7.17 | TCAActttcacttcAGT |
| 18_1 | 97.88 | 1.00 | 89.38 | 2.73 | 39.44 | 12.14 | TCAactttcacttCAG |
| 18_346 | 90.95 | 3.99 | 61.25 | 4.11 | −4.13 | 6.95 | TCaActttcactTCAG |
| 18_347 | 91.45 | 3.48 | 78.72 | 2.03 | 9.18 | 8.96 | TcAActttcactTCAG |
| 18_350 | 92.56 | 3.36 | 80.54 | 6.12 | 41.46 | 7.29 | TCAActttcactTcAG |
| 18_357 | 96.37 | 1.27 | 87.86 | 2.94 | 51.94 | 2.98 | TCaActttcacttCAG |
| 18_358 | 89.92 | 0.54 | 76.73 | 7.28 | 37.70 | 9.45 | TcaACtttcacttCAG |
| Combo 1 | 79.37 | 2.03 | 68.47 | 2.04 | 25.24 | 12.68 | |
| Combo 2 | 75.26 | 2.05 | 72.07 | 3.78 | 59.69 | 2.36 | |

From these data it can be seen that the best performing bispecific PAPD5/PAPD7 oligonucleotides have better effect in terms of HBsAg and HBeAg reduction with half the oligonucleotide concentration (20 μM) when compared to the combination treatments (2×20 μM).

### Example 6 Screening for In Vitro Efficacy of Stereodefined Antisense Oligonucleotides Targeting PAPD5 and PAPD7 in HeLa Cells

To expand the diversity around the motif sequences of SEQ ID NO: 18 even further, a library of stereodefined oligonucleotides was made based on the stereorandom parent compound with CMP ID NO 18_1.

Efficacy testing was performed in an in vitro experiment as described in Example 1, with the exception that the screening was conducted with 1 μM and some with 5 μM.

The relative PAPD5 mRNA and PAPD7 mRNA expression levels are shown in table 20 as % of the parent oligonucleotide i.e. the larger the value the better the inhibition.

TABLE 20
in vitro efficacy of stereodefined anti-PAPD5/PAPD7 compounds
(single experiment with duplex QPCR). PAPD5 and PAPD7 mRNA
levels are normalized to GUSB in HeLa cells and shown as % of
control (PBS treated cells).

| | % PAPD5 | | | | | | | | |
|---|---|---|---|---|---|---|---|---|---|
| | mRNA of control | | | | % PAPD7 mRNA of control | | | | |
| CMP ID | 1 μM | | 5 μM | | 1 μM | | 5 μM | | |
| NO | Avg | sd | Avg | sd | Avg | sd | Avg | sd | Stereodefinition |
| 18_1 | 100.0 | 6.3 | | | 100.0 | 3.4 | | | TCAactttcacttCAG |
| | | | | | | | | | XXXXXXXXXXXXXXXH |
| 18_32 | 87.0 | 5.1 | | | 94.7 | 0.9 | | | RSSRXXXXXXXXXXXH |
| 18_33 | 76.4 | NA | | | 89.7 | 1.7 | | | XRSSRXXXXXXXXXXH |
| 18_34 | 79.8 | 6.7 | | | 91.5 | 2.3 | | | XXRSSRXXXXXXXXXH |
| 18_35 | 70.0 | 10.8 | | | 86.7 | 3.8 | | | XXXRSSRXXXXXXXXH |
| 18_36 | 102.5 | 7.8 | | | 107.4 | 3.1 | | | XXXXRSSRXXXXXXXH |
| 18_37 | 88.8 | 7.6 | | | 95.1 | 4.5 | | | XXXXXRSSRXXXXXXH |
| 18_38 | 68.3 | 6.5 | | | 82.0 | 3.6 | | | XXXXXXRSSRXXXXXH |
| 18_39 | 87.2 | 5.7 | | | 93.8 | 5.0 | | | XXXXXXXRSSRXXXXH |
| 18_40 | 92.2 | 3.5 | | | 96.3 | 5.5 | | | XXXXXXXXRSSRXXXH |
| 18_41 | 81.1 | 1.3 | | | 95.2 | 7.6 | | | XXXXXXXXXRSSRXXH |
| 18_42 | 78.0 | 3.8 | | | 92.0 | 9.4 | | | XXXXXXXXXXRSSRXH |
| 18_43 | 80.4 | 3.4 | | | 92.7 | 3.6 | | | XXXXXXXXXXXRSSRH |
| 18_44 | 79.4 | 3.5 | | | 89.7 | 3.4 | | | XXXXXXXXXSSSSSRH |
| 18_45 | 75.2 | 8.2 | | | 88.7 | 2.4 | | | XXXXXXXXXRRRRRRH |
| 18_46 | 86.2 | 6.5 | | | 91.0 | 6.7 | | | XXXXXXXXXSSRRSRH |
| 18_47 | 79.7 | 6.2 | | | 85.7 | 1.5 | | | XXXXXXXXXSSSRSRH |
| 18_48 | 80.6 | 1.6 | | | 87.5 | 1.5 | | | XXXXXXXXXSSSRRSH |
| 18_49 | 79.9 | 3.2 | | | 101.8 | 6.5 | | | XXXXXXXXXSRSSSSH |
| 18_50 | 82.7 | 3.1 | | | 88.9 | 2.2 | | | XXXXXXXXXRSRSRSH |
| 18_51 | 78.0 | 5.7 | | | 90.2 | 2.9 | | | XXXXXXXXXSSSSRSH |
| 18_52 | 90.1 | 6.0 | | | 93.7 | 1.1 | | | XXXXXXXXXSSRRSSH |
| 18_53 | 82.7 | 8.7 | | | 90.7 | 3.2 | | | XXXXXXXXXRRSSSSH |
| 18_54 | 63.3 | 13.2 | | | 77.8 | 6.4 | | | XXXXXXXXXRSSRRRH |
| 18_55 | 73.9 | 6.2 | | | 90.9 | 1.6 | | | XXXXXXXXXSRRRRSH |
| 18_56 | 83.1 | 5.6 | | | 98.5 | 6.4 | | | XXXXXXXXXSSRSRRH |
| 18_57 | 73.4 | 6.8 | | | 89.6 | 8.2 | | | XXXXXXXXXRRRSRRH |
| 18_58 | 89.1 | 2.2 | | | 98.7 | 2.8 | | | XXXXXXXXXRRSRSRH |
| 18_59 | 73.2 | 8.5 | | | 91.7 | 2.5 | | | XXXXXXXXXSSRRRSH |
| 18_60 | 88.8 | 4.2 | | | 93.3 | 3.4 | | | XXXXXXXXXSRRSSSH |
| 18_61 | 77.0 | 13.6 | | | 81.6 | 13.7 | | | XXXXXXXXXRRRRRSH |
| 18_62 | 75.6 | 8.7 | | | 87.8 | 8.5 | | | XXXXXXXXXRRSSRRH |
| 18_63 | 74.8 | 5.0 | | | 85.5 | 1.4 | | | XXXXXXXXXRSRRRRH |
| 18_64 | 86.9 | 7.3 | | | 92.2 | 2.5 | | | XXXXXXXXXSRRRSSH |
| 18_65 | 77.8 | 10.3 | | | 89.0 | 7.4 | | | XXXXXXXXXSRSRSRH |
| 18_66 | 81.7 | 10.2 | | | 88.9 | 6.1 | | | XXXXXXXXXRSSSSRH |
| 18_67 | 77.6 | 7.4 | | | 81.1 | 4.7 | | | XXXXXXXXXSSSSRRH |
| 18_68 | 88.9 | 9.2 | | | 91.3 | 2.7 | | | XXXXXXXXXRRSSSRH |
| 18_69 | 77.8 | 3.8 | | | 89.9 | 4.0 | | | XXXXXXXXXRSSRRSH |
| 18_70 | 75.9 | 11.7 | | | 83.9 | 7.8 | | | XXXXXXXXXRSSSRRH |
| 18_71 | 84.2 | 6.7 | | | 88.7 | 1.4 | | | XXXXXXXXXSRRRRRH |
| 18_72 | 93.6 | 2.3 | | | 95.0 | 1.7 | | | XXXXXXXXXRRSRSSH |
| 18_73 | 90.5 | 4.3 | | | 92.4 | 2.9 | | | XXXXXXXXXRSRSSRH |
| 18_74 | 88.3 | 10.5 | | | 88.2 | 3.0 | | | XXXXXXXXXRSRSRRH |
| 18_75 | 85.2 | 7.1 | | | 89.0 | 3.1 | | | XXXXXXXXXSRRRSRH |
| 18_76 | 99.6 | 2.7 | | | 99.5 | 2.2 | | | XXXXXXXXXRRSRRSH |
| 18_77 | 87.4 | 1.5 | | | 87.2 | 1.8 | | | XXXXXXXXXSSSRRRH |
| 18_78 | 80.6 | 10.4 | | | 83.5 | 5.2 | | | XXXXXXXXXRSRRSRH |
| 18_79 | 89.3 | 6.8 | | | 98.7 | 3.4 | | | XXXXXXXXXSRRSRSH |
| 18_80 | 85.9 | 2.0 | | | 83.2 | 2.8 | | | XXXXXXXXXRRSRRRH |
| 18_81 | 92.4 | 5.0 | | | 84.1 | NA | | | XXXXXXXXXSRRSSRH |
| 18_82 | 86.8 | 3.4 | | | 89.8 | 3.0 | | | XXXXXXXXXSRSSSRH |
| 18_83 | 93.1 | 4.7 | | | 92.4 | 3.3 | | | XXXXXXXXXRSRRRSH |
| 18_84 | 91.1 | 4.9 | | | 93.4 | 5.2 | | | XXXXXXXXXSSSRSSH |
| 18_85 | 84.3 | 3.9 | | | 87.9 | 1.6 | | | XXXXXXXXXSSRSSRH |
| 18_86 | 86.2 | 8.1 | | | 84.6 | 2.2 | | | XXXXXXXXXRSSRSSH |
| 18_87 | 77.3 | 9.7 | | | 90.6 | 0.9 | | | XXXXXXXXXSRSSRSH |
| 18_88 | 85.8 | 5.4 | | | 92.4 | 3.0 | | | XXXXXXXXXSSSSSSH |
| 18_89 | 94.9 | 5.7 | | | 95.8 | 7.3 | | | XXXXXXXXXRSRRSSH |
| 18_90 | 91.2 | 6.3 | | | 92.9 | 2.3 | | | XXXXXXXXXRRRRSRH |
| 18_91 | 85.9 | 4.1 | | | 90.4 | 5.0 | | | XXXXXXXXXSSRSRSH |
| 18_92 | 84.7 | 6.5 | | | 90.1 | 9.3 | | | XXXXXXXXXRRRRSSH |
| 18_93 | 81.7 | 6.5 | | | 90.6 | 4.0 | | | XXXXXXXXXRSRSSSH |
| 18_94 | 82.2 | 7.7 | | | 82.9 | 8.0 | | | XXXXXXXXXRSSRSRH |
| 18_95 | 89.4 | 1.9 | | | 84.9 | 7.5 | | | XXXXXXXXXRRRSRSH |
| 18_96 | 80.1 | 3.7 | | | 85.0 | 5.9 | | | XXXXXXXXXRRSSRSH |
| 18_97 | 68.9 | 7.5 | | | 82.3 | 4.8 | | | XXXXXXXXXSRSSRRH |
| 18_98 | 81.7 | 4.1 | | | 93.9 | 6.9 | | | XXXXXXXXXSRRSRRH |
| 18_99 | 97.7 | 5.4 | | | 97.7 | 8.7 | | | XXXXXXXXXSRSRSSH |
| 18_100 | 77.5 | 3.7 | | | 85.4 | 4.1 | | | XXXXXXXXXSRSRRRH |
| 18_101 | 77.9 | 7.1 | | | 88.3 | 4.3 | | | XXXXXXXXXSSRSSSH |
| 18_102 | 77.3 | 6.3 | | | 93.0 | 2.8 | | | XXXXXXXXXRSSSSSH |
| 18_103 | 74.8 | 3.7 | | | 86.4 | 1.2 | | | XXXXXXXXXRSSSRSH |
| 18_104 | 90.3 | 6.1 | | | 91.5 | 2.3 | | | XXXXXXXXXRRRSSRH |
| 18_105 | 95.7 | 7.2 | | | 102.9 | 1.7 | | | XXXXXXXXXRRRSSSH |
| 18_106 | 79.7 | 5.4 | | | 85.7 | 1.2 | | | XXXXXXXXXSRSRRSH |
| 18_107 | 87.6 | 4.4 | | | 89.0 | 2.2 | | | XXXXXXXXXSSRRRRH |
| 18_108 | 86.4 | 10.6 | | | 95.3 | 4.0 | | | XXXXXXXXXXSSRSSH |
| 18_109 | 99.1 | 2.5 | | | 99.0 | 6.6 | | | XXXXXXXXXXRRRSSH |
| 18_110 | 91.1 | 5.4 | | | 93.1 | 3.5 | | | XXXXXXXXXXRRSSRH |
| 18_111 | 103.1 | 2.9 | | | 99.1 | 6.2 | | | XXXXXXXXXXRSSSRH |
| 18_112 | 96.5 | 2.7 | | | 90.7 | 2.5 | | | XXXXXXXXXXRRSRRH |
| 18_113 | 76.0 | 17.5 | | | 90.4 | 3.7 | | | XXXXXXXXXXSSSSRH |
| 18_114 | 86.9 | 3.4 | | | 88.8 | 4.5 | | | XXXXXXXXXXRRRRRH |
| 18_115 | 94.7 | 8.1 | | | 94.1 | 3.8 | | | XXXXXXXXXXSRSSSH |
| 18_116 | 79.8 | 4.1 | | | 83.7 | 2.6 | | | XXXXXXXXXXSSRSRH |
| 18_117 | 88.3 | 6.6 | | | 95.6 | 4.1 | | | XXXXXXXXXXRSSRSH |
| 18_118 | 83.6 | 7.9 | | | 86.8 | 2.1 | | | XXXXXXXXXXRSRRRH |
| 18_119 | 85.2 | 2.3 | | | 88.7 | 2.5 | | | XXXXXXXXXXSRRRRH |
| 18_120 | 86.2 | 6.8 | | | 91.9 | 0.7 | | | XXXXXXXXXXSRRRSH |
| 18_121 | 90.4 | 5.9 | | | 86.9 | 0.7 | | | XXXXXXXXXXSSSRSH |
| 18_122 | 74.2 | 8.8 | | | 79.5 | 7.8 | | | XXXXXXXXXXRSRSSH |
| 18_123 | 82.2 | 1.0 | | | 87.6 | 1.5 | | | XXXXXXXXXXSSSSSH |
| 18_124 | 91.0 | 12.7 | | | 111.4 | 11.9 | | | XXXXXXXXXXSRRSSH |
| 18_125 | 87.6 | 6.7 | | | 85.7 | 4.4 | | | XXXXXXXXXXRSRRSH |
| 18_126 | 81.5 | 7.1 | | | 85.5 | 1.9 | | | XXXXXXXXXXSSRRSH |
| 18_127 | 82.9 | 3.7 | | | 96.0 | 2.3 | | | XXXXXXXXXXRRRSRH |
| 18_128 | 79.0 | 3.7 | | | 83.5 | 4.3 | | | XXXXXXXXXXSRSRRH |
| 18_129 | 98.4 | NA | | | 91.7 | 6.2 | | | XXXXXXXXXXRRSRSH |
| 18_130 | 90.7 | 5.4 | | | 89.8 | 2.3 | | | XXXXXXXXXXRRSSSH |
| 18_131 | 82.2 | 6.1 | | | 89.6 | 1.0 | | | XXXXXXXXXXRSSSSH |
| 18_132 | 81.6 | 6.9 | | | 84.2 | 2.3 | | | XXXXXXXXXXRSSRRH |
| 18_133 | 88.9 | 4.1 | | | 94.5 | 4.0 | | | XXXXXXXXXXSRRSRH |
| 18_134 | 73.6 | 7.5 | | | 83.3 | 4.3 | | | XXXXXXXXXXSSRRRH |
| 18_135 | 86.6 | 10.3 | | | 91.0 | 7.1 | | | XXXXXXXXXXSRSSRH |
| 18_136 | 93.8 | 4.5 | | | 85.0 | 8.1 | | | XXXXXXXXXXRRRRSH |
| 18_137 | 100.6 | 6.4 | | | 83.2 | 7.2 | | | XXXXXXXXXXRSRSRH |
| 18_138 | 83.1 | 9.5 | | | 86.5 | 4.0 | | | XXXXXXXXXXSSSRRH |
| 18_139 | 82.4 | 10.8 | | | 87.3 | 2.9 | | | XXXXXXXXXXSRSRSH |
| 18_140 | 83.9 | 5.6 | | | 78.9 | 5.1 | | | SSRRRRSSSSSRSSRH |
| 18_141 | 96.7 | 9.9 | | | 89.2 | 13.8 | | | SSSSSRRRRRRSRRSH |
| 18_142 | 81.7 | 13.0 | | | 83.7 | 7.7 | | | SRSSRSSSRRRSRSRH |
| 18_143 | 86.4 | 11.5 | | | 80.3 | 11.7 | | | SRRSSSSRRSRRRRRH |
| 18_144 | 88.5 | 7.1 | | | 78.6 | 8.5 | | | SSRRSRSRSSSRSRRH |
| 18_145 | 75.2 | 12.2 | | | 78.4 | 3.9 | | | SSSRRRRSRRRSSRRH |
| 18_146 | 109.4 | 6.8 | | | 105.6 | 8.1 | | | RRSRSSRRSSSRRSSH |
| 18_147 | 82.8 | 7.1 | | | 80.3 | 2.9 | | | RSSRRRSSSRSSSRSH |
| 18_148 | 78.2 | 7.1 | | | 73.3 | 9.6 | | | SSSSRRRSRSSSRRSH |
| 18_149 | 78.5 | 3.9 | | | 77.1 | 14.5 | | | SSSRSSSSSSSRRRRH |
| 18_150 | 80.2 | 5.3 | | | 75.0 | 8.5 | | | SSSSRSSSSSSSSSSH |
| 18_151 | 65.6 | 21.5 | | | 73.0 | 9.1 | | | RRSRRRRRSSSSSSSH |
| 18_152 | 98.9 | 5.4 | | | 92.9 | 3.3 | | | RRRRSRSSRRRRSSSH |
| 18_153 | 92.1 | 9.5 | | | 93.2 | 3.1 | | | RRRRRSSRRRSRSSRH |
| 18_154 | 98.3 | 4.0 | | | 92.3 | 2.7 | | | SSRRRRSRSRSSRRSH |
| 18_155 | 77.4 | 8.1 | | | 82.0 | 3.8 | | | RSSSSSRSSRRSSSSH |
| 18_156 | 79.9 | 7.8 | | | 81.6 | 5.9 | | | RRRSSSSSRSRSRRSH |
| 18_157 | 76.8 | 4.3 | | | 82.6 | 3.5 | | | RSSSRSRSRRRSRRRH |
| 18_158 | 81.8 | 12.8 | | | 86.8 | 4.1 | | | RRSRRSSSRRRRRRSH |
| 18_159 | 76.4 | 12.4 | | | 77.9 | 2.8 | | | RRSSSSRSRSSSRSRH |
| 18_160 | 82.2 | 16.3 | | | 88.8 | 4.2 | | | RSSRSRSRSRSRSRRH |
| 18_161 | 76.4 | 14.9 | | | 77.9 | 4.9 | | | SRRRSSSSRSRSRSRH |
| 18_162 | 66.6 | 15.9 | | | 80.4 | 4.1 | | | SRSSSRRSRRRRSSRH |
| 18_163 | 76.8 | 14.0 | | | 85.3 | 2.9 | | | RSSRRRSRRSRSSRRH |
| 18_164 | 88.4 | 9.4 | | | 97.5 | 5.2 | | | SSRRRSSRSSRRRRSH |
| 18_165 | 75.1 | 14.9 | | | 85.2 | 3.0 | | | RSRSSRRSRRRSSSRH |
| 18_166 | 81.6 | 6.7 | | | 83.9 | 5.8 | | | RRRRSRRRSSRSRRSH |
| 18_167 | 74.4 | 11.7 | | | 77.5 | 4.5 | | | SRRRSSSRSRSSRRRH |
| 18_168 | 73.9 | 9.7 | | | 77.3 | 1.9 | | | SRSSRSSSSSRSRSSH |
| 18_169 | 73.7 | 15.1 | | | 86.2 | 1.1 | | | SSRRSRSSSSSRSSSH |
| 18_170 | 75.8 | 7.0 | | | 82.4 | 2.0 | | | SSRRRRRSRSRRSSSH |
| 18_171 | 97.4 | 2.3 | | | 98.5 | 3.3 | | | SSSRRSSRSRRRRRSH |
| 18_172 | 85.3 | 10.9 | | | 81.0 | 2.0 | | | RSSSSSSSRSRRRRRH |
| 18_173 | 88.5 | 10.0 | | | 92.5 | 1.4 | | | SSRSRSSRSSRRSRRH |
| 18_174 | 84.1 | 11.1 | | | 81.5 | 17.2 | | | SRSRSSSRRRSRRRSH |
| 18_175 | 72.7 | 6.6 | | | 79.1 | 1.1 | | | RRRRRRRSSRRSSSRH |
| 18_176 | 77.0 | 14.4 | | | 81.9 | 4.8 | | | SSRSRRRRRSRRSRSH |
| 18_177 | 81.9 | 5.6 | | | 79.9 | 10.1 | | | RRSRRRRRRSSRRRSH |
| 18_178 | 88.9 | 3.9 | | | 94.4 | 3.1 | | | SSSSRRRRRRRRRSRH |
| 18_179 | 87.6 | 11.8 | | | 81.5 | 8.6 | | | SRRRSSRRRSSRRRSH |
| 18_180 | 75.9 | 2.9 | | | 72.9 | 11.0 | | | SSSRRRRRSRRSSRRH |
| 18_181 | 85.3 | 11.1 | | | 86.7 | 1.9 | | | RRSRRSSSSRRRSSRH |
| 18_182 | 93.0 | 9.2 | | | 95.4 | 7.3 | | | SSRRSRSSRRRSSSSH |
| 18_183 | 83.6 | 12.3 | | | 80.6 | 5.2 | | | SSRSRRRRSSRSSSRH |
| 18_184 | 87.0 | 15.0 | | | 79.3 | 4.5 | | | RRRSRRSRSSRSRRRH |
| 18_185 | 98.7 | 4.6 | | | 96.8 | 1.7 | | | RSRSSRSRSRRSRSRH |
| 18_186 | 87.9 | 3.7 | | | 87.7 | 5.2 | | | SSSRRRRSSRRSRRRH |
| 18_187 | 99.1 | 3.5 | | | 99.8 | 2.3 | | | RSSRRSRRRRSRRRSH |
| 18_188 | 101.1 | 5.9 | | | 92.8 | 6.6 | | | SSSRRSSRSRSRSSSH |
| 18_189 | 106.9 | 4.2 | | | 105.0 | 3.1 | | | RSRSSSSRSSRRRSSH |
| 18_190 | 104.8 | 3.5 | | | 96.7 | 2.2 | | | SSSRSSSRSRRSRSSH |
| 18_191 | 87.7 | 10.4 | | | 84.9 | 7.8 | | | RSSRSSSSRSSSSSRH |
| 18_192 | 86.5 | 7.9 | | | 96.1 | 1.6 | | | RSSRRSSRSSSRRSRH |
| 18_193 | 76.5 | 8.0 | | | 80.4 | 3.2 | | | RSSRRSRSRRSSSSRH |
| 18_194 | 80.0 | 4.8 | | | 86.4 | 3.3 | | | RRSSSRRSRRRRSSSH |
| 18_195 | 100.4 | 8.3 | | | 99.3 | 1.6 | | | RRRRRSSRSRRSSSRH |
| 18_196 | 109.5 | 2.6 | | | 113.5 | 4.2 | | | SSSSRSRRRSSRRRSH |
| 18_197 | 82.6 | 1.9 | | | 81.0 | 4.8 | | | RSRRRRRRRRSSRSRH |
| 18_198 | 87.2 | 4.6 | | | 87.4 | 6.4 | | | RSRRSSSSRSSRSSRH |
| 18_199 | 80.9 | 2.8 | | | 91.5 | 1.0 | | | SSRRSRSSRRRSSSRH |
| 18_200 | 74.7 | 11.4 | | | 84.8 | 2.1 | | | RRRRSSSRRSRSRSSH |
| 18_201 | 73.5 | 13.7 | | | 82.0 | 1.3 | | | RSRRRRRRSRRSSRSH |
| 18_202 | 70.6 | 8.6 | | | 81.4 | 1.4 | | | SRRSRRRRRSRSSSSH |
| 18_203 | 69.8 | 9.5 | | | 73.8 | 1.4 | | | SRRSRRSSSRSSSSSH |
| 18_204 | 77.8 | 6.8 | | | 86.3 | 2.7 | | | SSSRRRRSRSRRRSSH |
| 18_205 | 73.4 | 4.2 | | | 77.8 | 2.6 | | | SSRSRSRSSSRSRSRH |
| 18_206 | 80.6 | 12.7 | | | 90.4 | 3.6 | | | SSSRRSRRSRRRSRSH |
| 18_207 | 67.8 | 7.5 | | | 74.3 | 2.6 | | | SRSSRRRSSSSSRRRH |
| 18_208 | 71.9 | 12.0 | | | 83.0 | 4.9 | | | RRSSRSSSSSSRSSRH |
| 18_209 | 74.0 | 5.5 | | | 83.7 | 3.4 | | | SRSSRRSSRSRRSRRH |
| 18_210 | 55.6 | 14.6 | 48.5 | 5.4 | 84.2 | 7.2 | 66.2 | 4.5 | RSRRSSRSRSSRRSSH |
| 18_211 | 60.5 | 11.1 | 52.4 | 6.7 | 84.4 | 6.7 | 76.4 | 6.8 | RSSSRRSRSSSRSSSH |
| 18_212 | 53.3 | 3.3 | 47.3 | 3.5 | 93.4 | 8.0 | 60.5 | 5.6 | SSSSSSSSRSRRRSSH |
| 18_213 | 43.0 | 8.3 | 26.1 | 6.0 | 72.4 | 10.7 | 38.3 | 8.4 | RRSSSSSSSRSSSRRH |
| 18_214 | 66.6 | 8.9 | 97.1 | 4.2 | 108.3 | 7.0 | 106.6 | 7.8 | SSSRRSSSSRRRRSSH |
| 18_215 | 61.0 | 11.2 | 59.9 | 8.2 | 98.3 | 10.7 | 76.0 | 11.9 | SSSRRRRRRSSSSRRH |
| 18_216 | 35.6 | 9.3 | 42.2 | 5.4 | 56.2 | 6.8 | 53.1 | 12.8 | RSRSRRRSSSRRRSRH |
| 18_217 | 37.6 | 8.9 | 73.8 | 8.8 | 65.0 | 6.4 | 79.6 | 8.0 | SSSSRRSRRRSSRRRH |
| 18_218 | 101.7 | 11.6 | 90.1 | 1.6 | 162.0 | 9.8 | 100.5 | 2.4 | RSSRRSSRSRRRSSSH |
| 18_219 | 70.9 | 10.8 | 75.5 | 3.7 | 97.0 | 9.1 | 93.3 | 4.9 | RRSSSSSRRRRSRRSH |
| 18_220 | 58.0 | 11.3 | 62.5 | 4.0 | 92.0 | 8.6 | 79.5 | 6.3 | RXXXXXXXXXXXXXXH |
| 18_221 | 66.8 | 8.8 | 89.8 | 4.1 | 101.2 | 11.1 | 109.1 | 6.9 | SXXXXXXXXXXXXXXH |
| 18_222 | 73.2 | 6.2 | 79.4 | 3.4 | 108.4 | 8.8 | 95.1 | 4.2 | XRXXXXXXXXXXXXXH |
| 18_223 | 84.1 | 9.0 | 98.4 | 4.9 | 134.3 | 6.6 | 134.7 | 5.5 | XSXXXXXXXXXXXXXH |
| 18_224 | 73.3 | 7.0 | 91.9 | 4.7 | 117.0 | 6.4 | 131.4 | 5.2 | XXRXXXXXXXXXXXXH |
| 18_225 | 76.5 | 9.3 | 94.3 | 7.7 | 110.1 | 6.0 | 108.4 | 7.6 | XXSXXXXXXXXXXXXH |
| 18_226 | 74.4 | 11.6 | 92.4 | 6.7 | 102.3 | 7.6 | 108.8 | 6.3 | XXXRXXXXXXXXXXXH |
| 18_227 | 83.1 | 11.6 | 109.9 | 8.4 | 99.1 | 14.1 | 111.2 | 6.9 | XXXSXXXXXXXXXXXH |
| 18_228 | 56.4 | 7.2 | 55.0 | 5.5 | 87.4 | 3.7 | 74.5 | 7.5 | XXXXRXXXXXXXXXXH |
| 18_229 | 69.4 | 6.2 | 81.4 | 4.4 | 113.1 | 4.6 | 104.9 | 7.4 | XXXXSXXXXXXXXXXH |
| 18_230 | 66.6 | 5.8 | 84.6 | 3.3 | 109.3 | 6.6 | 106.4 | 6.7 | XXXXXRXXXXXXXXXH |
| 18_231 | 80.7 | 2.7 | 109.0 | 1.1 | 114.1 | 5.6 | 120.8 | 4.9 | XXXXXSXXXXXXXXXH |
| 18_232 | 63.4 | 4.4 | 66.6 | 6.3 | 101.7 | 5.2 | 88.0 | 8.2 | XXXXXXRXXXXXXXXH |
| 18_233 | 68.3 | 3.1 | 96.4 | 8.0 | 102.4 | 6.5 | 120.3 | 6.6 | XXXXXXSXXXXXXXXH |
| 18_234 | 69.9 | 10.7 | 98.7 | 8.9 | 113.0 | 5.2 | 124.2 | 7.1 | XXXXXXXRXXXXXXXH |
| 18_235 | 68.6 | 16.7 | 82.3 | 7.5 | 91.1 | 12.4 | 90.3 | 9.2 | XXXXXXXSXXXXXXXH |
| 18_236 | 114.6 | 7.6 | 90.5 | 2.8 | 187.8 | 9.9 | 113.0 | 4.6 | XXXXXXXXRXXXXXXH |
| 18_237 | 66.4 | 13.5 | 66.6 | 7.3 | 117.3 | 12.3 | 93.2 | 7.3 | XXXXXXXXSXXXXXXH |
| 18_238 | 72.5 | 5.3 | 90.1 | 3.9 | 122.5 | 6.6 | 126.8 | 4.3 | XXXXXXXXXRXXXXXH |
| 18_239 | 39.8 | 3.0 | 20.9 | 5.7 | 67.2 | 6.4 | 29.2 | 2.1 | XXXXXXXXXSXXXXXH |
| 18_240 | 63.0 | 12.0 | 92.7 | 2.0 | 116.2 | 7.9 | 117.7 | 1.6 | XXXXXXXXXXRXXXXH |
| 18_241 | 65.1 | 15.1 | 75.4 | 4.4 | 105.9 | 19.9 | 104.8 | 5.0 | XXXXXXXXXXSXXXXH |
| 18_242 | 65.0 | 12.7 | 85.0 | 3.2 | 106.0 | 12.5 | 114.3 | 2.4 | XXXXXXXXXXXRXXXH |
| 18_243 | 145.2 | 7.8 | 112.0 | 6.0 | 180.8 | 6.4 | 118.8 | 6.5 | XXXXXXXXXXXSXXXH |
| 18_244 | 75.3 | 9.9 | 87.8 | 2.8 | 110.4 | 8.1 | 91.2 | 4.8 | XXXXXXXXXXXXRXXH |
| 18_245 | 81.7 | 8.6 | 63.6 | 5.6 | 100.3 | 5.9 | 79.2 | 1.9 | XXXXXXXXXXXXSXXH |
| 18_246 | 60.3 | 7.4 | 71.7 | 6.2 | 90.4 | 8.0 | 80.8 | 8.1 | XXXXXXXXXXXXXRXH |
| 18_247 | 70.3 | 8.0 | 90.4 | 6.4 | 108.4 | 7.5 | 94.4 | 8.1 | XXXXXXXXXXXXXSXH |
| 18_248 | 74.0 | 7.7 | 77.4 | 5.1 | 87.4 | 19.5 | 86.7 | 7.3 | XXXXXXXXXXXXXXRH |
| 18_249 | 74.8 | 4.9 | 88.2 | 5.4 | 114.8 | 5.6 | 109.7 | 6.4 | XXXXXXXXXXXXXXSH |

### Example 7: In Vitro EC50 and Efficacy of Selected Stereodefined Antisense Oligonucleotides in HeLa Cells

The EC50 and efficacy (KD) of the best performing oligonucleotides from Example 6 was determined using the same assay with the following oligonucleotide concentrations 33, 10.44, 3.33, 1.044, 0.33, 0.104, 0.033 and 0.01 μM.

EC 50, max KID (efficacy) of the PAPD5 and PAPD7 mRNA expression was calculated using R-function drm( ) from the drc package (v3.0-1) a four-parameter log-logistic function is fitted to the expression of the gene of interest as a function of oligonucleotide concentration to obtain a value for EC50 and maximum knock-down. The results are shown in Table 21.

TABLE 21
EC50 and Max KD of anti-PAPD5/PAPD7 compounds on PAPD5 and PAPD7 mRNA
expression in HeLa cells. CMP ID NO 18_1 is the stereorandom
parent compound.

| | PAPD5 | | | | PAPD7 | | | | |
|---|---|---|---|---|---|---|---|---|---|
| | Max KD % | | EC50 | | Max KD | | EC50 | | |
| CMP | of saline | | μM | | % of saline | | μM | | |
| ID NO | Avg | sd | Avg | sd | Avg | sd | Avg | sd | Stereodefined motif |
| 18_1 | 2.74 | 1.41 | 1.82 | 0.09 | 5.06 | 2.24 | 2.03 | 0.16 | TCAactttcacttCAG |
| | | | | | | | | | XXXXXXXXXXXXXXXH |
| 18_36 | 0.49 | 2.00 | 1.19 | 0.08 | 0.00 | 2.77 | 1.57 | 0.14 | XXXXRSSRXXXXXXXH |
| 18_76 | 1.83 | 5.88 | 3.18 | 0.54 | 1.12 | 7.32 | 3.38 | 0.69 | XXXXXXXXXRRSRRSH |
| 18_99 | 0.12 | 7.43 | 2.87 | 0.63 | 4.53 | 13.63 | 3.39 | 1.30 | XXXXXXXXXSRSRSSH |
| 18_109 | 2.46 | 3.84 | 1.59 | 0.20 | 2.66 | 4.77 | 2.04 | 0.32 | XXXXXXXXXXRRRSSH |
| 18_111 | 0.36 | 8.02 | 2.41 | 0.59 | 5.64 | 3.86 | 2.88 | 0.34 | XXXXXXXXXXRSSSRH |
| 18_124 | 0.00 | 8.02 | 1.76 | 0.45 | 0.00 | 4.30 | 2.27 | 0.28 | XXXXXXXXXXSRRSSH |
| 18_146 | 0.00 | 4.37 | 1.59 | 0.22 | 0.00 | 5.67 | 2.27 | 0.40 | RRSRSSRRSSSRRSSH |
| 18_171 | 0.00 | 3.47 | 1.44 | 0.17 | 0.00 | 5.90 | 2.24 | 0.41 | SSSRRSSRSRRRRRSH |
| 18_185 | 2.94 | 4.54 | 1.57 | 0.23 | 2.34 | 5.97 | 2.10 | 0.40 | RSRSSRSRSRRSRSRH |
| 18_187 | 0.00 | 2.50 | 1.73 | 0.14 | 0.00 | 6.11 | 2.27 | 0.40 | RSSRRSRRRRSRRRSH |
| 18_188 | 0.00 | 3.88 | 1.66 | 0.21 | 3.63 | 6.56 | 1.94 | 0.38 | SSSRRSSRSRSRSSSH |
| 18_190 | 3.56 | 5.01 | 2.59 | 0.41 | 7.41 | 6.38 | 3.11 | 0.62 | SSSRSSSRSRRSRSSH |
| 18_196 | 0.00 | 2.00 | 1.31 | 0.09 | 1.40 | 5.30 | 1.71 | 0.28 | SSSSRSRRRSSRRRSH |
| 18_223 | 0.00 | 3.36 | 1.40 | 0.16 | 1.15 | 4.84 | 1.83 | 0.28 | XSXXXXXXXXXXXXXH |
| 18_227 | 0.00 | 6.48 | 1.75 | 0.37 | 0.45 | 6.48 | 2.20 | 0.39 | XXXSXXXXXXXXXXXH |
| 18_231 | 0.00 | 3.57 | 1.37 | 0.17 | 0.00 | 4.34 | 2.13 | 0.28 | XXXXXSXXXXXXXXXH |
| 18_236 | 2.37 | 3.44 | 1.82 | 0.21 | 4.69 | 3.90 | 2.22 | 0.27 | XXXXXXXXRXXXXXXH |
| 18_243 | 0.15 | 5.38 | 2.38 | 0.37 | 5.18 | 8.67 | 2.52 | 0.66 | XXXXXXXXXXXSXXXH |

From these data it can be seen that improvements in EC50 and efficacy in relation to PAPD5 and PAPD7 knock down can be achieved both with stereodefined sub-libraries and with fully stereodefined compounds.

### Example 8: In Vitro Effect on HBV Infected ASGPR-dHepaRG Cells Using Selected Stereodefined Antisense Oligonucleotides Targeting PAPD5 and PAPD7

A selection of the most efficacious oligonucleotides from example 6 was tested for their effect on HBV propagation parameters in HBV infected dHepaRG-ASGPR cells.

The experiment was conducted as described in example 5.

The reduction of HBsAg and HBeAg levels are shown in table 22 and 23, the larger the value the larger the inhibition.

TABLE 22
in vitro efficacy on HBsAg of anti-PAPD5/PAPD7 compounds in three
concentrations (average of 3) in HBV infected ASGPR-dHepaRG
cells. CMP ID NO 18_1 is the stereorandom parent compound

| CMP ID | 20 μM | | 6.67 μM | | 2.22 μM | | |
|---|---|---|---|---|---|---|---|
| NO | Avg | sd | Avg | sd | Avg | sd | Stereodefined motif |
| 18_1 | 97.88 | 1.00 | 89.38 | 2.73 | 39.44 | 12.14 | TCAactttcacttCAG |
| | | | | | | | XXXXXXXXXXXXXXXH |
| 18_36 | 72.64 | 1.45 | 37.85 | 8.05 | 10.98 | 8.04 | XXXXRSSRXXXXXXXH |
| 18_76 | 40.85 | 34.07 | 2.07 | 19.39 | −15.02 | 23.15 | XXXXXXXXXRRSRRSH |
| 18_99 | 34.94 | 6.39 | −13.21 | 12.32 | −42.74 | 12.83 | XXXXXXXXXSRSRSSH |
| 18_105 | 82.12 | 2.60 | 74.93 | 3.30 | 19.30 | 7.25 | XXXXXXXXXRRRSSSH |
| 18_109 | 57.43 | 14.41 | 18.19 | 9.25 | 7.15 | 16.09 | XXXXXXXXXXRRRSSH |
| 18_111 | 28.98 | 6.10 | −10.71 | 7.93 | −30.92 | 15.15 | XXXXXXXXXXRSSSRH |
| 18_124 | 59.86 | 4.12 | 27.17 | 15.97 | −3.69 | 18.85 | XXXXXXXXXXSRRSSH |
| 18_146 | 62.69 | 6.93 | 44.31 | 4.08 | −19.52 | 12.39 | RRSRSSRRSSSRRSSH |
| 18_171 | 38.32 | 2.10 | −11.53 | 3.85 | −28.30 | 10.51 | SSSRRSSRSRRRRRSH |
| 18_185 | −20.73 | 17.60 | −19.59 | 14.46 | −4.32 | 7.01 | RSRSSRSRSRRSRSRH |
| 18_187 | 56.84 | 6.44 | 17.42 | 10.77 | −49.55 | 11.42 | RSSRRSRRRRSRRRSH |
| 18_188 | 59.41 | 12.82 | 25.09 | 16.54 | 6.76 | 20.56 | SSSRRSSRSRSRSSSH |
| 18_189 | 32.87 | 6.69 | −3.52 | 16.56 | −50.76 | 34.50 | RSRSSSSRSSRRRSSH |
| 18_190 | −53.00 | 16.64 | −57.27 | 12.78 | −69.75 | 14.40 | SSSRSSSRSRRSRSSH |
| 18_195 | 32.58 | 3.42 | −12.74 | 45.18 | −16.33 | 18.72 | RRRRRSSRSRRSSSRH |
| 18_196 | −17.72 | 3.29 | −36.50 | 9.00 | −49.29 | 11.33 | SSSSRSRRRSSRRRSH |
| 18_218 | 53.86 | 6.46 | 42.40 | 3.88 | 9.55 | 20.41 | RSSRRSSRSRRRSSSH |
| 18_223 | 83.06 | 2.73 | 62.17 | 11.58 | 15.29 | 11.02 | XSXXXXXXXXXXXXXH |
| 18_227 | 79.92 | 1.95 | 49.95 | 6.87 | −11.69 | 7.50 | XXXSXXXXXXXXXXXH |
| 18_231 | 83.13 | 1.45 | 69.70 | 3.35 | 37.16 | 11.77 | XXXXXSXXXXXXXXXH |
| 18_236 | 64.19 | 2.58 | 38.47 | 5.37 | −19.29 | 5.10 | XXXXXXXXRXXXXXXH |
| 18_243 | 82.96 | 1.85 | 67.55 | 3.06 | 26.96 | 10.36 | XXXXXXXXXXXSXXXH |
| Combo 1 | 79.37 | 2.03 | 68.47 | 2.04 | 25.24 | 12.68 | |
| Combo 2 | 75.26 | 2.05 | 72.07 | 3.78 | 59.69 | 2.36 | |

TABLE 23
in vitro efficacy on HBeAg of anti-PAPD5/PAPD7 compounds in
three concentrations (average of 3) in HBV infected
ASGPR-dHepaRG cells. CMP ID NO 18_1 is the
stereorandom parent compound

| CMP | 20 μM | | 6.67 μM | | 2.22 μM | | |
|---|---|---|---|---|---|---|---|
| ID NO | Avg | sd | Avg | sd | Avg | sd | Compound |
| 18_1 | 90.68 | 1.23 | 75.99 | 2.96 | 17.58 | 8.44 | TCAactttcacttCAG |
| | | | | | | | XXXXXXXXXXXXXXXH |
| 18_36 | 61.56 | 2.27 | 32.88 | 7.00 | 13.90 | 2.63 | XXXXRSSRXXXXXXXH |
| 18_76 | 42.45 | 24.97 | 12.44 | 4.58 | 5.05 | 11.65 | XXXXXXXXXRRSRRSH |
| 18_99 | 29.44 | 4.44 | −5.01 | 7.61 | −15.22 | 8.54 | XXXXXXXXXSRSRSSH |
| 18_105 | 77.20 | 2.93 | 63.83 | 3.75 | 17.89 | 6.08 | XXXXXXXXXRRRSSSH |
| 18_109 | 50.97 | 12.79 | 18.65 | 7.96 | 18.34 | 10.47 | XXXXXXXXXXRRRSSH |
| 18_111 | 26.62 | 5.65 | 5.57 | 6.76 | −5.32 | 8.48 | XXXXXXXXXXRSSSRH |
| 18_124 | 52.84 | 6.90 | 26.44 | 13.62 | 8.76 | 13.32 | XXXXXXXXXXSRRSSH |
| 18_146 | 57.25 | 5.51 | 32.84 | 4.19 | −5.83 | 9.16 | RRSRSSRRSSSRRSSH |
| 18_171 | 31.41 | 2.24 | −0.52 | 0.38 | −5.55 | 4.51 | SSSRRSSRSRRRRRSH |
| 18_185 | 3.01 | 9.20 | 0.38 | 6.33 | 6.86 | 2.17 | RSRSSRSRSRRSRSRH |
| 18_187 | 45.26 | 5.54 | 14.19 | 7.61 | −7.36 | 5.03 | RSSRRSRRRRSRRRSH |
| 18_188 | 51.94 | 10.97 | 26.12 | 10.92 | 15.12 | 17.90 | SSSRRSSRSRSRSSSH |
| 18_189 | 32.71 | 4.45 | 3.59 | 7.73 | −20.18 | 13.54 | RSRSSSSRSSRRRSSH |
| 18_190 | −8.26 | 5.56 | −19.34 | 5.60 | −23.56 | 3.06 | SSSRSSSRSRRSRSSH |
| 18_195 | 33.37 | 4.40 | 6.47 | 23.36 | −3.00 | 7.15 | RRRRRSSRSRRSSSRH |
| 18_196 | 8.16 | 3.13 | −5.42 | 9.08 | −16.04 | 9.21 | SSSSRSRRRSSRRRSH |
| 18_218 | 52.20 | 7.32 | 38.24 | 6.77 | 9.85 | 11.45 | RSSRRSSRSRRRSSSH |
| 18_223 | 79.06 | 3.79 | 53.28 | 3.42 | 15.60 | 12.30 | XSXXXXXXXXXXXXXH |
| 18_227 | 76.98 | 5.26 | 39.75 | 9.09 | −0.96 | 3.34 | XXXSXXXXXXXXXXXH |
| 18_231 | 72.79 | 4.62 | 54.88 | 2.74 | 25.58 | 8.29 | XXXXXSXXXXXXXXXH |
| 18_236 | 59.69 | 3.81 | 33.06 | 7.16 | −0.33 | 4.37 | XXXXXXXXRXXXXXXH |
| 18_243 | 79.05 | 1.15 | 53.54 | 2.97 | 21.12 | 7.39 | XXXXXXXXXXXSXXXH |
| Combo 1 | 72.08 | 0.75 | 58.03 | 2.25 | 21.27 | 8.25 | |
| Combo 2 | 71.77 | 4.54 | 67.54 | 3.72 | 50.53 | 5.82 | |

### Example 9: In Vitro Effect on HBV Infected ASGPR-dHepaRG Cells Using Selected GalNAc Conjugated Antisense Oligonucleotides Targeting PAPD5 and PAPD7

A selection of the most efficacious oligonucleotides from Example 1 were conjugated to a GalNAc conjugate moiety and tested for their effect on HBV propagation parameters in HBV infected ASGPR-dHepaRG cells.

The assessment of the EC50 and efficacy (KD) on HBsAg and HBeAg of the GalNAc conjugated oligonucleotides was performed as described in Example 2 using HBV infected ASGPR-dHepaRG cells and without comparative oligonucleotides. The results are shown in Table 24.

In addition to the procedure in example 2 the harvested cells were washed once in PBS and lysed in MagNA Pure lysis buffer (Roche #05467535001) and stored at −80° C. RNA was extracted using MagNA Pure “96 Cellular RNA Large Volume Kit” (Roche #05467535001) and PAPD5 and PAPD7 mRNA expression levels were determined as described in Materials and Methods section, Real-time PCR for PAPD5 and PAPD7. EC50 and efficacy (KD) was calculated using the R-function drm( ) from the drc package (v3.0-1) a four-parameter log-logistic function is fitted to the expression of the gene of interest as a function of oligonucleotide concentration to obtain a value for EC50 and maximum knock-down. The results are shown in Table 24A

TABLE 24
EC50 and Max KD of anti-PAPD5/PAPD7 compounds on HBsAg and HBeAg
(average of 3) in HBV infected ASGPR-dHepaRG cells.

| | HBeAg | | | | HBsAg | | | | |
|---|---|---|---|---|---|---|---|---|---|
| | Max KD | | | | Max KD | | | | |
| CMP | % of | | EC50 | | % of | | EC50 | | |
| ID | saline | | nM | | saline | | nM | | |
| NO | Avg | sd | Avg | sd | Avg | sd | Avg | sd | Compound |
| 20_12 | 6.1 | 1.0 | 127.7 | 10.1 | 7.7 | 1.6 | 87.0 | 17.4 | GN2-C6ₒcₒaₒTCAactttcacttCAG |
| 20_13 | 0.8 | 0.3 | 65.1 | 1.3 | 2.5 | 1.0 | 72.4 | 3.5 | GN2-C6ₒcₒaₒTCAActttcactTCAG |
| 20_14 | 0.3 | 1.1 | 43.2 | 3.4 | 1.2 | 1.3 | 58.5 | 5.1 | GN2-C6ₒcₒaₒTCAActttcacttCAG |
| 20_15 | 0.0 | 0.7 | 45.3 | 6.1 | 0.4 | 1.7 | 37.8 | 11.2 | GN2-C6ₒcₒaₒTCAActttcacTtCAG |
| 20_16 | 3.9 | 2.9 | 58.2 | 6.6 | 1.9 | 2.4 | 84.2 | 11.6 | GN2-C6ₒcₒaₒTCAACtttcacttCAG |
| 20_17 | 5.9 | 1.9 | 83.8 | 11.8 | 11.2 | 1.7 | 110.4 | 14.3 | GN2-C6ₒcₒaₒTCAACtttcacttcAG |
| 20_18 | 6.5 | 2.1 | 75.6 | 34.3 | 13.9 | 2.4 | 77.8 | 33.2 | GN2-C6ₒcₒaₒTCAActttcacttcAG |
| 20_19 | 0.0 | 7.3 | 76.3 | 81.9 | 11.4 | 4.2 | 106.9 | 26.9 | GN2-C6ₒcₒaₒTCAactttcactTCAG |
| 20_20 | 0.0 | 6.1 | 79.6 | 59.4 | 9.2 | 2.4 | 135.0 | 16.2 | GN2-C6ₒcₒaₒTcAACtttcacttcAG |
| 20_21 | 1.8 | 2.4 | 41.5 | 8.7 | 7.8 | 2.6 | 74.9 | 17.6 | GN2-C6ₒcₒaₒTcAACtttcacttcAG |
| 20_22 | 7.2 | 1.2 | 60.6 | 6.8 | 10.7 | 0.7 | 126.7 | 6.9 | GN2-C6ₒcₒaₒTCaACtttcacttcAG |
| 21_2 | 14.6 | 55 | 79.2 | 40.8 | 18.8 | 3.3 | 125.9 | 23.6 | GN2-C6ₒcₒaₒTCAActttcacttCaGT |

From these data it can be seen that by conjugating a GalNAc moiety to the oligonucleotide the EC50 values are improved at least 40 fold (note the current table is in nM whereas table 14 is in μM). For example is the HBsAg reduction of compound 20_15 (GalNAc conjugated) improved 176 fold over compound 18_05 (naked version of 20_15).

TABLE 24A
in vitro efficacy and potency (EC50) of GalNAc conjugated
anti-PAPD5/PAPD7 compounds. PAPD5 and PAPD7 mRNA levels
are normalized to GUSB in ASGPR-dHepaRG cells and shown
as % of control (PBS treated cells).

| | PAPD5 | | | | PAPD7 | | | |
|---|---|---|---|---|---|---|---|---|
| CMP | Max KD | | EC50 | | Max KD | | EC50 | |
| ID | % of saline | | μM | | % of saline | | μM | |
| NO | Avg | sd | Avg | sd | Avg | sd | Avg | sd |
| 20_12 | 1.9 | 0.95 | 0.032 | 0.002 | 1.6 | 1.59 | 0.030 | 0.003 |
| 20_13 | 17 | 1.93 | 0.045 | 0.009 | 17 | 1.57 | 0.038 | 0.006 |
| 20_14 | 5.2 | 1.24 | 0.024 | 0.008 | 2.9 | 1.47 | 0.003 | 0.002 |
| 20_15 | 11 | 1.45 | 0.002 | 0.002 | 8.5 | 0.99 | 0.001 | 0.001 |
| 20_16 | 10 | 1.20 | 0.046 | 0.006 | 11 | 1.18 | 0.041 | 0.005 |
| 20_17 | 5.2 | 2.29 | 0.022 | 0.012 | 4.3 | 2.05 | 0.037 | 0.013 |
| 20_18 | 5.4 | 1.14 | 0.047 | 0.006 | 2 | 1.27 | 0.014 | 0.007 |
| 20_19 | 4.7 | 1.68 | 0.048 | 0.009 | 6.5 | 1.54 | 0.041 | 0.009 |
| 20_20 | 9.3 | 1.33 | 0.047 | 0.005 | 4.7 | 2.17 | 0.019 | 0.012 |
| 20_21 | 6.2 | 1.30 | 0.043 | 0.006 | 4.4 | 2.78 | 0.020 | 0.008 |
| 20_22 | 4.7 | 1.29 | 0.044 | 0.008 | 5.4 | 2.68 | 0.048 | 0.010 |
| 21_2 | 12 | 1.12 | 0.075 | 0.005 | 12 | 3.41 | 0.052 | 0.013 |

From these data it can be seen that the majority of the selected GalNAc conjugated oligonucleotides targeting PAPD5 and PAPD7 are capable of reducing the mRNA levels to below 10%.

### Example 10: Screening for In Vitro Efficacy of Antisense Oligonucleotides Targeting Papd5 and Papd7 in dHepaRG Cells

The oligonucleotides screened for PAPD5 and PAPD7 knock down in HeLa cells (Example 1 and 3) were screened in dHepaRG cells to demonstrate efficient knock down in a liver cell line.

dHepaRG cells were cultured as described in the Materials and Method section. The following oligonucleotide concentrations 50, 15.81, 5.00, 1.58, 0.50, 0.16, 0.05, and 0.016 μM were used in a final culture volume of 100 μl/well. The cells were harvested 6 days after addition of oligonucleotide compounds and RNA was extracted using the PureLink Pro 96 RNA Purification kit (Ambion) according to the manufacturer's instructions.

PAPD5 and PAPD7 mRNA levels were analysed by Real-time PCR as described in the Materials and Method section. EC 50, max KD (efficacy) of the PAPD5 and PAPD7 mRNA expression was calculated using the R-function drm( ) from the drc package (v3.0-1) a four-parameter log-logistic function is fitted to the expression of the gene of interest as a function of oligonucleotide concentration to obtain a value for EC50 and maximum knock-down.

The results are shown in table 25.

TABLE 25
EC50 and Max KD of anti-PAPD5/PAPD7 compounds on
PAPD5 and PAPD7 mRNA expression in dHepaRG cells.

| | PAPD5 | | | | PAPD7 | | | | |
|---|---|---|---|---|---|---|---|---|---|
| | Max KD | | | | Max KD | | | | |
| | % of | | EC50 | | % of | | EC50 | | |
| CMP | saline | | μM | | saline | | μM | | |
| ID NO | Avg | sd | Avg | sd | Avg | sd | Avg | sd | Compound |
| 17_103 | 11.0 | 6.1 | 1.7 | 0.4 | 2.2 | 39.9 | 7.6 | 13.3 | TcAActttcactTCaGT |
| 17_111 | 7.5 | 8.7 | 2.2 | 0.8 | 0.0 | 38.7 | 6.4 | 11.7 | TCaactttcactTcAGT |
| 17_119 | 5.3 | 16.1 | 1.8 | 1.1 | 2.1 | 15.7 | 3.8 | 2.2 | TCAActttcactTcaGT |
| 17_129 | 11.5 | 5.5 | 1.5 | 0.4 | 0.0 | 31.2 | 5.0 | 6.2 | TCAactttcacttCAGT |
| 17_132 | 9.8 | 10.0 | 3.2 | 1.3 | 13.2 | 13.2 | 6.6 | 3.5 | TCaActttcacttCAGT |
| 17_135 | 4.1 | 3.6 | 1.1 | 0.1 | 0.0 | 32.7 | 4.0 | 4.3 | TCaactttcacttCAGT |
| 17_137 | 0.0 | 7.5 | 3.5 | 0.9 | 16.6 | 8.2 | 5.0 | 1.5 | TcAActttcacttCAGT |
| 17_139 | 5.3 | 8.3 | 2.3 | 0.7 | 5.7 | 19.1 | 7.7 | 4.9 | TcAactttcacttCAGT |
| 17_144 | 6.0 | 8.0 | 1.4 | 0.4 | 0.0 | 12.7 | 2.8 | 1.3 | TCAactttcacttCaGT |
| 17_157 | 8.2 | 4.6 | 3.1 | 0.5 | 0.0 | 16.2 | 8.8 | 4.9 | TCAActttcacttcAGT |
| 18_1 | 0.0 | 7.8 | 1.6 | 0.4 | 0.0 | 8.7 | 3.8 | 1.2 | TCAactttcacttCAG |
| 18_6 | 10.1 | 9.2 | 2.5 | 0.9 | 0.0 | 19.8 | 5.8 | 4.2 | TCaactttcacTtCAG |
| 18_10 | 13.4 | 15.6 | 1.5 | 1.0 | 10.1 | 15.1 | 4.1 | 2.3 | TCAActttcactTCAG |
| 18_12 | 8.8 | 7.4 | 1.9 | 0.6 | 13.3 | 8.9 | 4.6 | 1.6 | TCAactttcactTCAG |
| 18_15 | 0.0 | 35.4 | 4.7 | 6.0 | 34.8 | 11.8 | 4.8 | 2.3 | TcAACtttcactTcAG |
| 18_18 | 0.0 | 27.1 | 2.6 | 2.7 | 25.0 | 7.3 | 5.4 | 1.5 | TCAACtttcacttCAG |
| 18_19 | 0.0 | 7.0 | 2.8 | 0.7 | 0.0 | 18.1 | 1.2 | 1.0 | TCAActttcacttCAG |
| 18_20 | 11.9 | 10.6 | 4.2 | 1.8 | 0.0 | 64.2 | 9.3 | 22.5 | TCAaCtttcacttCAG |
| 18_21 | 21.9 | 7.0 | 4.4 | 1.3 | 0.0 | 40.5 | 16.0 | 25.6 | TCaaCtttcacttCAG |
| 18_23 | 8.8 | 10.8 | 3.0 | 1.2 | 0.0 | 32.5 | 3.5 | 4.1 | TCAACtttcacttcAG |
| 18_24 | 13.5 | 5.9 | 3.3 | 0.8 | 23.3 | 6.2 | 3.4 | 1.0 | TCAActttcacttcAG |
| 18_25 | 13.0 | 11.4 | 3.0 | 1.3 | 9.4 | 18.7 | 5.0 | 3.3 | TCAaCtttcacttcAG |
| 18_27 | 7.9 | 9.2 | 2.7 | 0.9 | 19.2 | 7.5 | 3.3 | 1.0 | TCaACtttcacttcAG |
| 18_28 | 13.4 | 11.3 | 4.7 | 2.1 | 19.1 | 5.8 | 4.6 | 1.1 | TCaaCtttcacttcAG |
| 18_30 | 9.9 | 7.4 | 5.1 | 1.2 | 0.0 | 14.4 | 7.1 | 3.5 | TcAACtttcacttcAG |
| 18_346 | 8.1 | 8.9 | 1.5 | 0.6 | 0.0 | 19.1 | 3.9 | 2.5 | TCaActttcactTCAG |
| 18_347 | 9.2 | 15.0 | 1.6 | 1.0 | 0.0 | 24.0 | 4.3 | 3.6 | TcAActttcactTCAG |
| 18_350 | 8.5 | 6.3 | 1.8 | 0.5 | 0.0 | 24.4 | 3.4 | 2.6 | TCAActttcactTcAG |
| 18_357 | 0.0 | 10.0 | 4.5 | 1.6 | 0.0 | 25.5 | 8.1 | 6.5 | TCaActttcacttCAG |
| 18_358 | 0.0 | 19.3 | 3.9 | 2.5 | 29.9 | 9.2 | 4.3 | 1.8 | TcaACtttcacttCAG |

From these data it can be seen that an effective target reduction can also be archived in a hepatocyte derived cell line.

### Example 11: Screening for In Vitro Efficacy of Stereodefined Antisense Oligonucleotides Targeting PAPD5 and PAPD7 in dHepaRG Cells

The stereodefined oligonucleotides screened for PAPD5 and PAPD7 knock down in HeLa cells (Example 7) were screened in dHepaRG cells to demonstrate efficient knock down in a liver cell line.

The screening was conducted as described in example 10 with the following oligonucleotide concentrations 33, 10.44, 3.33, 1.044, 0.33, 0.104, 0.033 and 0.01 μM.

PAPD5 and PAPD7 mRNA levels were analysed by Real-time PCR as described in the Materials and Method section. EC 50, max KD (efficacy) of the PAPD5 and PAPD7 mRNA expression was calculated using the R-function drm( ) from the drc package (v3.0-1) a four-parameter log-logistic function is fitted to the expression of the gene of interest as a function of oligonucleotide concentration to obtain a value for EC50 and maximum knock-down.

The results are shown in table 26.

TABLE 26
EC50 and Max KD of anti-PAPD5/PAPD7 stereodefined compounds
on PAPD5 and PAPD7 mRNA expression in dHepaRG cells

| | PAPD5 | | | | PAPD7 | | | | |
|---|---|---|---|---|---|---|---|---|---|
| | Max KD | | | | Max KD | | | | |
| | % of | | EC50 | | % of | | EC50 | | |
| CMP | saline | | μM | | saline | | μM | | |
| ID NO | Avg | sd | Avg | sd | Avg | sd | Avg | sd | Stereodefined motif |
| 18_1 | 0.0 | 7.75 | 1.6 | 0.43 | 0.0 | 8.71 | 3.8 | 1.16 | TCAactttcacttCAG |
| | | | | | | | | | XXXXXXXXXXXXXXXH |
| 18_36 | 3.6 | 2.33 | 1.3 | 0.11 | 0.0 | 7.23 | 1.6 | 0.34 | XXXXRSSRXXXXXXXH |
| 18_76 | 0.0 | 18.65 | 6.3 | 3.60 | 11.6 | 11.23 | 6.5 | 2.36 | XXXXXXXXXRRSRRSH |
| 18_99 | 9.4 | 6.99 | 5.7 | 1.40 | 13.7 | 18.67 | 7.2 | 4.94 | XXXXXXXXXSRSRSSH |
| 18_109 | 4.0 | 9.74 | 2.3 | 0.75 | 6.4 | 15.14 | 3.4 | 1.73 | XXXXXXXXXXRRRSSH |
| 18_111 | 7.4 | 16.00 | 3.0 | 1.61 | 12.6 | 14.95 | 4.4 | 2.12 | XXXXXXXXXXRSSSRH |
| 18_124 | 7.0 | 29.13 | 1.7 | 1.81 | 6.3 | 14.24 | 3.7 | 1.55 | XXXXXXXXXXSRRSSH |
| 18_146 | 1.7 | 19.93 | 1.8 | 1.19 | 12.3 | 20.51 | 4.9 | 3.39 | RRSRSSRRSSSRRSSH |
| 18_171 | 3.9 | 6.86 | 1.7 | 0.40 | 0.0 | 16.12 | 3.0 | 1.52 | SSSRRSSRSRRRRRSH |
| 18_185 | 0.0 | 14.48 | 2.6 | 1.19 | 10.4 | 9.76 | 4.1 | 1.28 | RSRSSRSRSRRSRSRH |
| 18_187 | 5.2 | 8.79 | 1.5 | 0.45 | 2.9 | 5.11 | 2.0 | 0.35 | RSSRRSRRRRSRRRSH |
| 18_188 | 7.5 | 4.82 | 1.5 | 0.28 | 12.2 | 10.13 | 1.7 | 0.63 | SSSRRSSRSRSRSSSH |
| 18_190 | 0.0 | 27.66 | 8.1 | 8.27 | 30.4 | 10.66 | 4.1 | 1.95 | SSSRSSSRSRRSRSSH |
| 18_196 | 9.0 | 8.92 | 1.8 | 0.62 | 19.7 | 8.01 | 1.5 | 0.51 | SSSSRSRRRSSRRRSH |
| 18_223 | 11.2 | 10.00 | 1.4 | 0.62 | 19.9 | 6.90 | 2.5 | 0.75 | XSXXXXXXXXXXXXXH |
| 18_227 | 6.4 | 20.21 | 1.7 | 1.19 | 10.8 | 10.55 | 3.2 | 1.15 | XXXSXXXXXXXXXXXH |
| 18_231 | 10.2 | 5.89 | 1.3 | 0.30 | 9.9 | 6.10 | 2.1 | 0.44 | XXXXXSXXXXXXXXXH |
| 18_236 | 10.8 | 6.26 | 3.1 | 0.59 | 15.3 | 6.47 | 3.3 | 0.64 | XXXXXXXXRXXXXXXH |
| 18_243 | 6.0 | 9.15 | 1.8 | 0.52 | 26.9 | 3.26 | 1.9 | 0.24 | XXXXXXXXXXXSXXXH |

From these data it can be seen that stereo defined oligonucleotides also are effective in target reduction in a hepatocyte derived cell line.

### Example 12: In Vitro Effect on HBV Infected ASGPR-dHepaRG Cells Using Selected GalNAc Conjugated Antisense Oligonucleotides Targeting PAPD5 and PAPD7

A selection of the most efficacious oligonucleotides from example 5 were conjugated to a GalNAc conjugate moiety and tested for their effect on HBV propagation parameters in HBV infected ASGPR-dHepaRG cells.

For comparative purposes the antisense oligonucleotides of the invention were compared to GalNAc conjugated versions of the he HBV targeting oligonucleotides shown in table 13, the GalNAc conjugated versions are shown in Table 13A.

TABLE 13A
Comparative HBV targeting oligonucleotides

| Description | Compound | SEQ ID NO | Reference |
|---|---|---|---|
| HBV targeting 1 | GN2ₒcₒaₒAGCgaagtgcacaCGG | 29 | WO2015/173208 |
| HBV targeting 2 | GN2ₒcₒaₒGCGtaaagagaGG | 30 | WO2015/173208 |

The assessment of the EC50 and efficacy (KD) on HBsAg and HBeAg of the GalNAc conjugated oligonucleotides was performed as described in Example 2 using HBV infected ASGPR-dHepaRG cells. The results are shown in Table 27.

In addition to the procedure in example 2 the harvested cells were washed once in PBS and lysed in MagNA Pure lysis buffer (Roche #05467535001) and stored at −80° C. RNA was extracted using MagNA Pure “96 Cellular RNA Large Volume Kit” (Roche #05467535001) and PAPD5 and PAPD7 mRNA expression levels were determined as described in Materials and Methods section, Real-time PCR for PAPD5 and PAPD7. EC50 and efficacy (KD) was calculated using the R-function drm( ) from the drc package (v3.0-1) a four-parameter log-logistic function is fitted to the expression of the gene of interest as a function of oligonucleotide concentration to obtain a value for EC50 and maximum knock-down. The results are shown in Table 27A.

TABLE 27
EC50 and Max KD of anti-PAPD5/PAPD7 compounds on
HBsAg and HBeAg (average of 3) in HBV infected ASGPR-
dHepaRG cells.

| | HBeAg | | | | HBsAg | | | | |
|---|---|---|---|---|---|---|---|---|---|
| | Max KD | | | | Max KD | | | | |
| CMP | % of | | EC50 | | % of | | EC50 | | |
| ID | saline | | μM | | saline | | μM | | |
| NO | Avg | sd | Avg | sd | Avg | sd | Avg | sd | Compound |
| HBV1 | 26.4 | 3.6 | 0.124 | 0.026 | 39.6 | 7.3 | 0.220 | 0.104 | GN2- |
| | | | | | | | | | C6caAGCgaagtgcacaCGG |
| HBV2 | 31.3 | 4.2 | 0.233 | 0.373 | 33.2 | 4.8 | 0.391 | 0.119 | GN2- |
| | | | | | | | | | C6caGCGtaaagagaGG |
| 21_26 | 11.4 | 15.7 | 0.175 | 0.113 | 18.1 | 8.9 | 0.201 | 0.070 | GN2- |
| | | | | | | | | | C6caTcAActttcactTCAGT |
| 21_27 | 18.5 | 6.2 | 0.128 | 0.041 | 23.3 | 8.1 | 0.192 | 0.068 | GN2- |
| | | | | | | | | | C6caTCAActttcactTCaGT |
| 21_33 | 28.4 | 19.3 | 0.247 | 0.133 | 33.2 | 10.5 | 0.242 | 0.106 | GN2- |
| | | | | | | | | | C6caTcAActttcacttCAGT |
| 21_34 | 17.6 | 5.5 | 0.083 | 0.037 | 27.3 | 3.7 | 0.085 | 0.091 | GN2- |
| | | | | | | | | | C6caTcAactttcacttCAGT |
| 21_36 | 13.8 | 6.0 | 0.086 | 0.156 | 20.6 | 9.6 | 0.193 | 0.086 | GN2- |
| | | | | | | | | | C6caTCAActttcacttcAGT |
| 20_12 | 0.0 | 2.6 | 0.073 | 0.088 | 9.9 | 1.9 | 0.057 | 0.005 | GN2- |
| | | | | | | | | | C6caTCAactttcacttCAG |
| 20_35 | 3.2 | 10.4 | 0.080 | 0.166 | 9.7 | 6.6 | 0.085 | 0.143 | GN2- |
| | | | | | | | | | C6caTCaActttcactTCAG |
| 20_36 | 3.7 | 4.0 | 0.082 | 0.001 | 3.9 | 3.1 | 0.082 | 0.014 | GN2- |
| | | | | | | | | | C6caTcAActttcactTCAG |
| 20_30 | 4.8 | 5.6 | 0.107 | 0.031 | 2.3 | 4.2 | 0.137 | 0.032 | GN2- |
| | | | | | | | | | C6caTCaActttcacttCAG |

The compounds indicated in the the table have phosphodiester linkages in the ca dinucleotide following the C6 linker as it is indicated in table 10.

TABLE 27A
in vitro efficacy and potency (EC50) of GalNAc conjugated
anti-PAPD5/PAPD7 compounds. PAPD5 and PAPD7 mRNA levels
are normalized to GUSB in ASGPR-dHepaRG cells and shown
as % of control (PBS treated cells).

| | PAPD5 | | | | PAPD7 | | | |
|---|---|---|---|---|---|---|---|---|
| CMP | Max KD | | EC50 | | Max KD | | EC50 | |
| ID | % of saline | | μM | | % of saline | | μM | |
| NO | Avg | sd | Avg | sd | Avg | sd | Avg | sd |
| HBV1 | 58 | 9.26 | Inf | 10.00 | 76 | 11.5 | 0.780 | 10.000 |
| HBV2 | 59 | 43.5 | Inf | 24000 | 82 | 7.47 | Inf | 10.000 |
| 21_26 | 11 | 2.01 | 0.080 | 0.010 | 14 | 2.01 | 0.059 | 0.010 |
| 21_27 | 7.8 | 1.04 | 0.056 | 0.004 | 14 | 3.4 | 0.076 | 0.018 |
| 21_33 | 8.4 | 1.2 | 0.050 | 0.005 | 14 | 2.16 | 0.075 | 0.009 |
| 21_34 | 4.8 | 1.05 | 0.065 | 0.004 | 9.4 | 1.75 | 0.047 | 0.006 |
| 21_36 | 3.9 | 1.04 | 0.087 | 0.005 | 2.4 | 5.85 | 0.033 | 0.025 |
| 20_12 | 1.6 | 1.05 | 0.034 | 0.004 | 3.6 | 1.79 | 0.040 | 0.006 |
| 20_35 | 6.7 | 1.51 | 0.038 | 0.006 | 8.4 | 1.81 | 0.054 | 0.008 |
| 20_36 | 3.4 | 1.48 | 0.037 | 0.004 | 6.9 | 4.35 | 0.082 | 0.018 |
| 20_30 | 1.9 | 1.06 | 0.035 | 0.003 | 4.9 | 5.8 | 0.040 | 0.019 |

Inf = EC50 could not be calculated due to lack in dose response.

As expected the two HBV targeting molecules had very insignificant effect on PAPD5 and PAPD7, their HBsAg and HBeAg effects are therefore not connected to their ability to reduce PAPD5 or PAPD7. The reminder of the tested compound show target reduction below 85% and EC50 values below 0.09 μM, which correlate well with the effects seen on HBsAg and HBeAg in table 27.

### Example 13 In Vitro Effect on HBV Infected PHH Cells Using Selected GalNAc Conjugated Antisense Oligonucleotides Targeting PAPD5 and PAPD7

A selection of GalNAc conjugated oligonucleotides were further tested in HBV infected primary human hepatocytes (see materials and method section; PHH natural infection assay) to illustrate efficacy in an in vitro system with a natural ASGPR expression. The oligonucleotide concentrations used were three-fold serial dilutions (20.00, 6.67, 2.22, 0.74, 0.25, 0.08, 0.03, 0.01 μM oligonucleotide).

EC 50, max KD (efficacy) of the HBV propagation parameters HBsAg and HBeAg was calculated using the R-function drm( ) from the drc package (v3.0-1) a four-parameter log-logistic function is fitted to the expression of the gene of interest as a function of oligonucleotide concentration to obtain a value for EC50 and maximum reduction. The results are shown in Table 28.

EC 50, max KD (efficacy) of the PAPD5 and PAPD7 mRNA expression was calculated using the same algorithm. The results are shown in Table 28A.

TABLE 28
EC50 and Max KD of anti-PAPD5/PAPD7 compounds on
HBsAg and HBeAg (average of 3) in HBV infected PHH cells.

| | HBsAg | | | | HBeAg | | | | |
|---|---|---|---|---|---|---|---|---|---|
| | Max KD | | | | Max KD | | | | |
| CMP | % of | | EC50 | | % of | | EC50 | | |
| ID | saline | | μM | | saline | | μM | | |
| NO | Avg | sd | Avg | sd | Avg | sd | Avg | sd | Compound |
| 20_13 | 11.8 | 4.1 | 0.078 | 0.179 | 9.0 | 2.3 | 0.078 | 0.010 | GN2- |
| | | | | | | | | | C6caTCAActttcactTCAG |
| 20_14 | 11.9 | 1.9 | 0.062 | 0.006 | 13.1 | 1.8 | 0.063 | 0.006 | GN2- |
| | | | | | | | | | C6caTCAActttcacttCAG |
| 20_12 | 17.0 | 2.1 | 0.054 | 0.006 | 24.4 | 1.3 | 0.075 | 0.005 | GN2- |
| | | | | | | | | | C6caTCAactttcacttCAG |
| 20_15 | 9.5 | 1.4 | 0.017 | 0.003 | 11.2 | 2.4 | 0.029 | 0.006 | GN2- |
| | | | | | | | | | C6caTCAActttcacTtCAG |
| 20_16 | 16.7 | 1.9 | 0.098 | 0.010 | 19.5 | 3.4 | 0.180 | 0.031 | GN2- |
| | | | | | | | | | C6caTCAACtttcacttCAG |
| 20_17 | 16.9 | 2.1 | 0.068 | 0.011 | 26.0 | 3.0 | 0.119 | 0.024 | GN2- |
| | | | | | | | | | C6caTCAACtttcacttcAG |
| 20_18 | 13.2 | 1.9 | 0.066 | 0.008 | 19.2 | 1.0 | 0.070 | 0.004 | GN2-C6caTCAActttcacttcAG |
| 20_20 | 14.8 | 5.0 | 0.087 | 0.022 | 18.8 | 4.3 | 0.168 | 0.043 | GN2- |
| | | | | | | | | | C6caTcAACtttcactTcAG |

The compounds indicated in the the table have phosphodiester linkages in the ca dinucleotide following the C6 linker as it is indicated in table 10.

From these data it can be seen that the selected GalNAc conjugated oligonucleotides targeting PAPD5 and PAPD7 are capable of reducing HBV antigen secretion in infected primary human hepatocytes.

TABLE 28A
in vitro efficacy and potency (EC50) of GalNAc conjugated
anti-PAPD5/PAPD7 compounds. PAPD5 and PAPD7 mRNA
levels are normalized to GUSB in PPH cells and shown
as % of control (PBS treated cells).

| | PAPD5 | | | | PAPD7 | | | |
|---|---|---|---|---|---|---|---|---|
| CMP | Max KD | | EC50 | | Max KD | | EC50 | |
| ID | % of saline | | μM | | % of saline | | μM | |
| NO | Avg | sd | Avg | sd | Avg | sd | Avg | sd |
| 20_13 | 0 | 6.28 | 0.030 | 0.028 | 0 | 10.4 | 0.018 | 0.034 |
| 20_14 | 3.6 | 1.92 | 0.026 | 0.007 | 0 | 8.63 | 0.011 | 0.020 |
| 20_12 | 4.2 | 3.41 | 0.033 | 0.009 | 2.9 | 5.31 | 0.007 | 0.012 |
| 20_15 | 0 | 6.37 | 0.001 | 0.001 | 0 | 8.93 | 0.033 | 0.061 |
| 20_16 | 11 | 2.67 | 0.094 | 0.016 | 1.8 | 6.2 | 0.016 | 0.016 |
| 20_17 | 91 | 4.6 | 4.200 | 0.270 | 13 | 6.05 | 0.039 | 0.022 |
| 20_18 | NA | NA | NA | NA | 6.7 | 7.11 | 0.015 | 0.016 |
| 20_20 | 11 | 3.19 | 0.045 | 0.012 | 13 | 7.9 | 0.004 | 0.015 |

NA = not assessed due to technical error

From these data it can be seen that the selected GalNAc conjugated oligonucleotides targeting PAPD5 and PAPD7 are capable of reducing their targets to 11% or lower, with the exception of compound 20_17 that appears to have very little effect on PAPD5 mRNA, while maintain the effect on PAPD7 mRNA.

### Example 14 Screening for In Vitro Efficacy of Antisense Oligonucleotides Targeting Human And Mouse PAPD5 and PAPD7 (Bispecific) in HeLa Cells and PMH Cells

An oligonucleotide screen was performed using gapmer oligonucleotides targeting the human and mouse transcripts of PAPD5 and PAPD7 (table 5) in the human HeLa cell line and in primary mouse hepatocytes (PMH).

The screening in HeLa cells was conducted as described in Example 1 with a 25 μM concentration.

The screening in PMH cells was conducted as described in the “Materials and methods” section under “Primary mouse Hepatocytes” using 5 μM oligonucleotide.

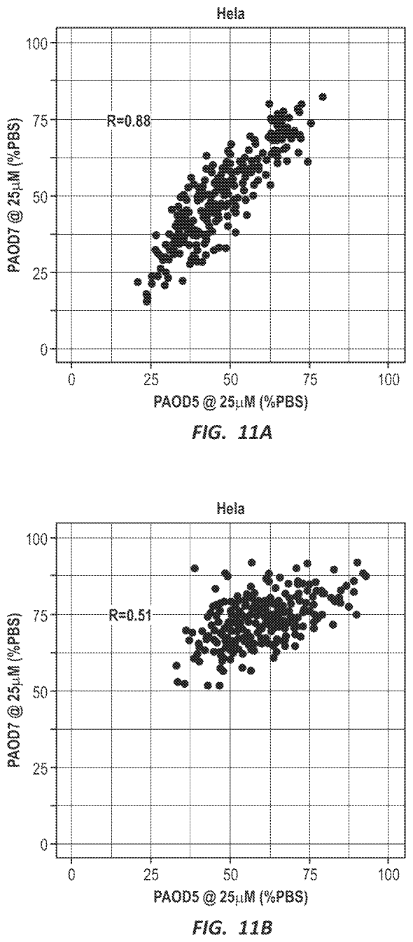

FIG. 11 shows the results of the screen, each dot represents a compound from table 5 and it's ability to reduce PAPD7 mRNA (Y axis) and PAPD5 mRNA (X axis). In the HeLa cells (human) there is a good correlation between PAPD5 and PAPD7 mRNA reduction, whereas in the PMH (mouse) cells it appears that the reduction of PAPD7 mRNA is not very efficient compared to the PAPD5 mRNA reduction.

A plausible explanation of the modest inhibition of PAPD7 mRNA in the mouse hepatocytes is that the primary spliced mRNA transcript of PAPD7 expressed in primary mouse hepatocytes has a transcription start site downstream of the binding site of the oligonucleotides. This was not identified until a whole transcriptome shotgun sequencing (RNAseq) was performed on the primary mouse hepatocytes.

### Example 15: In Vitro Effect on HBV Infected ASGPR-dHepaRG Cells Using Selected GalNAc Conjugated Antisense Oligonucleotides Targeting PAPD5 and PAPD7

A further selection of oligonucleotides from example 2 and 5 were conjugated to a GalNAc conjugate moiety and tested for their effect on HBV propagation parameters in HBV infected ASGPR-dHepaRG cells.

The assessment of the EC50 and efficacy (KD) on HBsAg and HBeAg of the GalNAc conjugated oligonucleotides was performed as described in Example 2 using HBV infected ASGPR-dHepaRG cells. The results are shown in Table 29.

In addition to the procedure in example 2 the harvested cells were washed once in PBS and lysed in MagNA Pure lysis buffer (Roche #05467535001) and stored at −80° C. RNA was extracted using MagNA Pure “96 Cellular RNA Large Volume Kit” (Roche #05467535001) and PAPD5 and PAPD7 mRNA expression levels were determined as described in Materials and Methods section, Real-time PCR for PAPD5 and PAPD7. EC50 and efficacy (KD) was calculated using the R-function drm( ) from the drc package (v3.0-1) a four-parameter log-logistic function is fitted to the expression of the gene of interest as a function of oligonucleotide concentration to obtain a value for EC50 and maximum knock-down. The results are shown in Table 29A.

TABLE 29
EC50 and Max KD of anti-PAPD5/PAPD7 compounds on HBsAg and
HBeAg (average of 3) in HBV infected ASGPR-dHepaRG cells.

| | HBeAg | | HBsAg | | |
|---|---|---|---|---|---|
| | Max KD | EC50 | Max KD | EC50 | |
| CMP ID | % of saline | μM | % of saline | μM | |
| NO | Avg | Avg | Avg | Avg | Compound |
| 20_12 | 8.12 | 0.05 | 9.59 | 0.05 | GN2-C6ocoaoTCAactttcacttCAG |
| 21_20 | 26.60 | 0.32 | 27.25 | 0.32 | GN2-C6ocoaoTcAactttcactTcAGT |
| 21_21 | 21.08 | 0.12 | 24.20 | 0.17 | GN2-C6ocoaoTcAActttcacttCaGT |
| 21_22 | 42.22 | 0.52 | 40.26 | 1.43 | GN2-C6ocoaoTCAactttcacttcAGT |
| 20_31 | 17.80 | 0.18 | 17.19 | 0.29 | GN2-C6ocoaoTCaactttcactTCAG |
| 20_32 | 1.20 | 0.07 | 10.25 | 0.08 | GN2-C6ocoaoTCAaCtttcacttCAG |
| 20_33 | 15.30 | 0.13 | 22.90 | 0.17 | GN2-C6ocoaoTCaaCtttcacttCAG |
| 20_34 | 12.51 | 0.07 | 14.65 | 0.07 | GN2-C6ocoaoTCAaCtttcacttcAG |
| 21_41 | 26.52 | 4.25 | 37.88 | 4.84 | GN2-C6ocoaoTCaactttcactTCAGT |
| 21_44 | 35.05 | 0.11 | 37.69 | 0.23 | GN2-C6ocoaoTCaActttcacttCAGT |
| 20_40 | 0.00 | 0.06 | 6.26 | 0.09 | GN2-C6ocoaoTcaACtttcacttCAG |
| 20_39 | 0.00 | 0.05 | 16.47 | 0.07 | GN2-C6ocoaoTCAActttcactTcAG |
| 21_42 | 23.75 | 0.13 | 26.69 | 0.17 | GN2-C6ocoaoTCaActttcactTCaGT |
| 21_43 | 8.92 | 0.08 | 16.60 | 0.16 | GN2-C6ocoaoTCAActttcactTcaGT |

TABLE 29A
in vitro efficacy and potency (EC50) of GalNAc conjugated
anti-PAPD5/PAPD7 compounds. PAPD5 and PAPD7 mRNA levels
are normalized to GUSB in ASGPR-dHepaRG cells and shown
as % of control (PBS treated cells).

| | PAPD5 | | | | PAPD7 | | | |
|---|---|---|---|---|---|---|---|---|
| CMP | Max KD | | EC50 | | Max KD | | EC50 | |
| ID | % of saline | | μM | | % of saline | | μM | |
| NO | Avg | sd | Avg | sd | Avg | sd | Avg | sd |
| 20_12 | 1.8 | 1.31 | 0.043 | 0.005 | 1.5 | 1.42 | 0.027 | 0.005 |
| 21_20 | 6.8 | 1.48 | 0.076 | 0.009 | 12 | 3.21 | 0.096 | 0.018 |
| 21_21 | 12 | 1.38 | 0.035 | 0.007 | 16 | 4.3 | 0.009 | 0.019 |
| 21_22 | 4.7 | 0.723 | 0.044 | 0.003 | 5.1 | 2.2 | 0.044 | 0.009 |
| 20_31 | 5.9 | 1.55 | 0.056 | 0.009 | 6.3 | 1.57 | 0.048 | 0.008 |
| 20_32 | 8 | 1.37 | 0.058 | 0.007 | 6.2 | 2.09 | 0.027 | 0.020 |
| 20_33 | 11 | 1.28 | 0.084 | 0.008 | 5.4 | 3.57 | 0.001 | 0.002 |
| 20_34 | 6.8 | 1.87 | 0.046 | 0.011 | 8.2 | 2.2 | 0.044 | 0.007 |
| 21_41 | 35 | 4.51 | 0.097 | 0.045 | 37 | 5.74 | 0.220 | 0.096 |
| 21_44 | 10 | 1.79 | 0.120 | 0.016 | 21 | 2.2 | 0.140 | 0.024 |
| 20_40 | 4.2 | 1.38 | 0.041 | 0.006 | 7.3 | 1.11 | 0.047 | 0.004 |
| 20_39 | 5.4 | 1.98 | 0.026 | 0.011 | 8 | 3.15 | 0.025 | 0.014 |
| 21_42 | 16 | 1.8 | 0.098 | 0.011 | 16 | 2.46 | 0.063 | 0.010 |
| 21_43 | 5.8 | 1.31 | 0.059 | 0.008 | 11 | 2.31 | 0.044 | 0.010 |

### Example 16 Effect on HBsAg Expression from Chromosomally Integrated HBV DNA Using Selected Bispecific PAPD5 and PAPD7 Targeting Oligonucleotides

In the current experiment it was tested whether a selection of GalNAc conjugated anti-PAPD5/7 oligonucleotides with good potency towards PAPD5 and PAPD7 were capable of reducing HBs antigen and mRNA expression from the human hepatocellular carcinoma cell line Hep3B which secrete HBs antigen from chromasomally integrated HBV DNA.

Hep3B cells (Knowles et al. 1980. Science 209 pp. 497-499) were purchased from ATCC (ATCC HB-8064) and cultured in Eagle's minimum essential medium (EMEM) supplemented with 10% FBS. The cells were plated on collagen coated 96-well plates at a concentration of 1.5×10⁵ cells per well and cultured at 37° C. in a humidified atmosphere with 5% CO₂. One day after seeding the cells oligonucleotide was added to the cells using concentrations starting at 20 μM and three-fold serial dilutions thereof (20.00, 6.67, 2.22, 0.74, 0.25, 0.08, 0.03, 0.01 μM oligonucleotide). The treatment was repeated with a medium change on day 4 and day 7. At day 11 the supernatants were harvested for HBsAg measurement (performed as described in the Materials and Method section under HBV antigen measurements) and the cells were washed once with PBS and 2000 μl MagNA Pure lysis buffer was added to each well and plates were stored at −80° C. for RNA extraction.

Intracellular mRNA was extracted from lysed Hep3B cells using a MagNA Pure robot and the MagNA Pure 96 Cellular RNA Large Volume Kit (Roche, #05467535001) according to the manufacturer's protocol. PAPD5 and PAPD7 mRNA was quantified in technical duplicate by separate RT-qPCRs using a QuantStudio 12K Flex (Applied Biosystems), the TaqMan RNA-to-CT 1-Step Kit (Applied Biosystems, #4392938), Human ACTB endogenous control (Applied Biosystems, #4310881E), and PAPD5 and PAPD7 mRNA Taqman primers and reagents (Life Technologies, assay ID Hs00900790_m1 (PAPD5) and Hs00173159_m1 (PAPD7) and custom assay ID APMFW4G (Small HBs)). The qPCR was performed using the following settings: UDG incubation (15 min, 48° C.), enzyme activation (10 min, 95° C.) and qPCR (40 cycles with 15 sec, 95° C. for denaturation and 1 min, 60° C. for annealing and extension).

EC 50 and max KD (Max efficacy in % of saline) of the HBsAg, HBs mRNA, PAPD5 and PAPD7 reductions was calculated using using GraphPad Prism 7.02 non line fit. The results are shown in Table 30 and 31.

TABLE 30
EC50 and Max KD of anti-PAPD5/PAPD7 compounds on chromosomal
integrated HBs mRNA and HBsAg expressed from the chromosomal
integrant (average of 3 biological replicates and 2 technical
duplicates) in Hep3B cells.

| | HBsAg | | HBs mRNA | | |
|---|---|---|---|---|---|
| | Max KD | EC50 | Max KD % | EC50 | |
| CMP ID | % of saline | μM | of saline | μM | |
| NO | Avg | Avg | Avg | Avg | Compound |
| 20_12 | 26.51 | 0.37 | 49.94 | 0.33 | GN2-C6ocoaoTCAactttcacttCAG |
| 20_21 | 45.17 | 1.55 | 52.85 | 0.27 | GN2-C6ocoaoTcAACtttcacttcAG |
| 20_20 | NA | >20 | 67.68 | 0.13 | GN2-C6ocoaoTcAACtttcactTcAG |
| 21_34 | 82.3 | NA | 86.73 | NA | GN2-C6ocoaoTcAactttcacttCAGT |
| 20_13 | 14.25 | 0.43 | 27.67 | 0.19 | GN2-C6ocoaoTCAActttcactTCAG |
| 20_14 | 19.60 | 0.39 | 35.97 | 0.15 | GN2-C6ocoaoTCAActttcacttCAG |
| 21_33 | 56.68 | 5.33 | 68.22 | 0.02 | GN2-C6ocoaoTcAActttcacttCAGT |

NA = not applicable

TABLE 31
EC50 and Max KD of anti-PAPD5/PAPD7 compounds on on
PAPD5 and PAPD7 mRNA expression (average of 3 biological
replicates and 2 technical duplicates) in Hep3B cells.

| | PAPD5 | | PAPD7 | | |
|---|---|---|---|---|---|
| | mRNA | | mRNA | | |
| | Max KD | EC50 | Max KD | EC50 | |
| CMP ID | % of saline | μM | % of saline | μM | |
| NO | Avg | Avg | Avg | Avg | Compound |
| 20_12 | 10.83 | 0.16 | 14.08 | 0.18 | GN2-C6ocoaoTCAactttcacttCAG |
| 20_21 | 15.57 | 0.33 | 15.72 | 0.35 | GN2-C6ocoaoTcAACtttcacttcAG |
| 20_20 | 27.34 | 0.17 | 33.46 | 0.22 | GN2-C6ocoaoTcAACtttcactTcAG |
| 21_34 | 21.51 | 0.43 | 33.83 | 0.46 | GN2-C6ocoaoTcAactttcacttCAGT |
| 20_13 | 9.76 | 0.11 | 12.31 | 0.17 | GN2-C6ocoaoTCAActttcactTCAG |
| 20_14 | 5.17 | 0.15 | 7.78 | 0.17 | GN2-C6ocoaoTCAActttcacttCAG |
| 21_33 | 21.19 | 0.16 | 30.13 | 0.31 | GN2-C6ocoaoTcAActttcacttCAGT |

From these data it can be seen that 4 out of the 7 tested oligonucleotides are capable of reducing HBsAg and HBs mRNA expression from an intergrated HBs fragment to less than 55% of the saline control.

### Example 17 Effect of a Selected Bispecific PAPD5 and PAPD7 Targeting Oligonucleotide in Non-Human Primates

Inhibition of PAPD5 and PAPD7 mRNA expression in the liver of cynomolgus macaques was quantified by RNA-sequencing. The animals were treated once-weekly with either saline or 1, 3, or 10 mg/kg/week with compound ID NO 20_12 for 4 weeks (6 animals per group, 5 doses total at days 1, 8, 15, 22 and 29) and sacrificed on day 29 (4 weeks post dosing). In parallel, animals were treated once-weekly with either saline or 10 mg/kg/week of compound ID NO 20_12), again for 4 weeks, for a total of 5 doses, but with a 4 week recovery period and sacrificed at day 56 (4 week dosing+4 weeks recovery).

Liver samples were collected in RNA-Later (Qiagen cat. 76104) within 20 min after exsanguination. Approximately 10 mg of tissue were lysed in 800 microL Magnapure lysis buffer (Roche) using the Tissue Lyser II (Qiagen). 350 microL aliquots of lysates were then transferred into the Magnapure 96 Deep Well Plate and processed automatically. RNA was quantified by absorption spectroscopy (Nanodrop, ThermoFischer) and RNA integrity (as per RNA integrity number, RIN) was controlled by microfluidic capillary array electrophoresis using the Agilent Bioanalyzer 2100 with RNA 6000 Nanochips (Agilent cat. 5067-1511).

For the construction of barcoded cDNA libraries, 400 ng total RNA aliquots were used as input for the TruSeq™ Stranded Total RNA kit (Illumina cat. 20020598) in conjunction with the Ribo-Zero™ Gold rRNA Removal Kit (Illumina cat. MRZG12324). The size distribution of the libraries was estimated by electrophoresis using the Agilent High Sensitivity DNA kit (cat. 5067-4627). The libraries were quantified using the KAPA Library Quantification qRT-PCR kit (Kapa Biosystems cat. KK4824). The libraries were pooled at equimolar concentrations and diluted to 11 pM prior to loading onto a flow cell of the Illumina HiSeq 4000 sequencer as follows The libraries were extended using the HiSeq PE Rapid Cluster Kit v2 (Illumina cat. PE-402-4002). The flow cells carrying amplified clusters were sequenced using paired-end reads (50-base pairs) with the TruSeq Rapid SBS Kit-HS (Illumina cat. FC-402-4001). Real time image analysis and base calling were performed using the HiSeq Sequencing Control Software (HCS). CASAVA software version 1.8 was used for production of FASTQ files of sequence read pairs.

The lowest library size obtained was 17 million read pairs and the highest was 114 million read pairs. On average there were 50 million read pairs per sample and the median was at 47 million read pairs per sample. Read pairs of each library were aligned to the Cynomolgus transcripts from the RefSeq/NCBI database using the GSNAP program to generate gene-level raw counts. These were normalized to the respective library size (for inter-samples comparisons) and for each transcript the data were further normalized to the respective transcript length (for inter-transcript comparisons). For all samples this generated transcript-level expression in normalized units RPKMs (Reads Per Kilobase of transcript, per Million mapped reads). The values for PAPD5 and PAPD7 in the treated animals were normalized to the saline-treated animals, at the corresponding timepoint the results are shown in table 32.

TABLE 32
PAPD5 and PAPD7 mRNA expression in liver of cynomolgus
monkeys treated with CMP ID NO 20_12.

| | | | PAPD5 | PAPD5 | PAPD7 | PAPD7 |
|---|---|---|---|---|---|---|
| | | | mRNA % | mRNA | mRNA % | mRNA |
| | | | of saline*, | geo- | of saline*, | geo- |
| | | | geo- | metric | geo- | metric |
| CMP ID | | | metric | SD | metric | SD |
| NO 20_12 | Dose | | mean | factor | mean | factor |
| After 4 wk | Saline | | 100 | 1.35 | 100 | 1.24 |
| dosing | 1 | mg/kg | 24.2 | 1.31 | 46.4 | 1.30 |
| | 3 | mg/kg | 18.2 | 1.23 | 37.1 | 1.40 |
| | 10 | mg/kg | 19.3 | 1.34 | 33.8 | 1.22 |
| After 4 wk | Saline | | 100 | 1.13 | 100 | 1.26 |
| dosing + 4 | 10 | mg/kg | 21.8 | 1.65 | 45.5 | 1.31 |
| wk follow up | | | | | | |

*normalized to control animals for same time-point

Relatively to the respective vehicle control group, the results show down-regulation of PAPD5 and PAPD7 mRNAs in liver, both in the main group animals and in recovery animals, at all tested dose levels of CMP ID NO 20_12. The down-regulation of PAPD5 mRNA appeared saturated in the liver with around 80% at 3 and 10 mg/kg. The down-regulation of PAPD7 mRNA was dose-related, reaching 66% reduction of mRNA at 10 mg/kg. In the recovery animals dosed with 10 mg/kg/week, the down-regulation of PAPD5 mRNA was 78%. For PAPD7 mRNA, the down-regulation reached 55%. The latter data indicates that the PAPD5 and PAPD7 mRNA inhibition persisted in the liver at least for 4 weeks after last dose.

### Example 18 Effect on HBsAg and HBeAg in HBV Infected Mice Following Administration Of PAPD5 and PAPD7 Targeting Oligonucleotides

The present study sets out to show an in vivo effect on the HBV propagation parameters when reducing the PAPD5 and PAPD7 transcripts in the AAV/HBV mouse model.

Example 14 and FIG. 11B showed that it was challenging to target both PAPD5 and PAPD7 in a mouse cell line using a single oligonucleotide. In the present study a combination of two oligonucleotides, one targeting mouse PAPD5 (CMP ID NO: 22_1) and one targeting mouse PAPD7 CMP ID NO: 22_1) listed in table 33, has therefore been used.

TABLE 33
Oligonucleotides targeting mouse PAPD5 (SEQ ID NO: 5)
or mouse PAPD7 (SEQ ID NO: 6)

| | | | | | |
|---|---|---|---|---|---|
| SEQ | | | | | |
| ID | | | | CMP ID | |
| NO | Motif sequence | Start | End | NO | Compound |

| | | | | |
|---|---|---|---|---|
| 22 | caacataagtctacacatcc | SEQ ID NO: 5 | 22_1 | 5′-GN2- |

| | | |
|---|---|---|
| 60034 | 60051 | C6ₒcₒaₒACataagtctacacATCC |

| | | | | |
|---|---|---|---|---|
| 23 | cagttttaccgattcatca | SEQ ID NO: 6 | 23_1 | 5′-GN2- |

| | | |
|---|---|---|
| 10684 | 10700 | C6ₒcₒaₒGTtttaccgattcATCA |

GN2 represents the trivalent GalNAc cluster shown in FIG. 2, C6 represents an amino alkyl group with 6 carbons, capital letters represent beta-D-oxy LNA nucleosides, lowercase letters represent DNA nucleosides, all LNA C are 5-methyl cytosine, subscript o represent a phosphodiester nucleoside linkage and unless otherwise indicated internucleoside linkages are phosphorothioate internucleoside linkages.

The AAV/HBV mouse model described in the Materials and Method section was used. Mice (3 pr. group) were dosed subcutaneously with a single dose of 10 mg/kg of each of compounds 22_1 and 23_1 (two separate injections 6 hours apart) or with 5 ml/kg saline (control) on day 0. HBsAg and HBeAg in serum was measured every 3 days using the methods described in the “Materials and Methods” section. To measure target knockdown two intermediate groups of mice were sacrificed on day 3 and day 14 and the remaining mice were sacrificed on day 27. After scarification their liver was removed following PBS perfusion. The perfused liver was cut in smaller pieces and directly frozen.

mRNA was extracted from the frozen liver pieces by adding them to 2 ml tubes containing ceramic beads and 1 ml MagNA Pure lysis buffer (Roche #05467535001). The liver pieces were homogenized using the TissueLyser (Qiagen). RNA was isolated from the tissue homogenates using MagNA Pure “96 Cellular RNA Large Volume Kit” (Roche #05467535001). The lysates may be stored at −80° C. PAPD5 and PAPD7 mRNA was measured essentially using qPCR as described in the Materials and Method section, with the following change in the TaqMan primer assay, which was performed with the following two assay (ThermoFisher Scientific):

| | |
|---|---|
| Mouse GUSB | Mm1197698_m1 |
| Mouse PAPD5 | Mm1244121_m1 |
| Mouse PAPD7 | Mm1349513_m1 |
| Mouse TBP | Mm00446971_m1 |
| Mouse PAPD5 | Mm_011244125m1 |
| Mouse PAPD7 | Mm1349513_m1 |

GUSB and TBP are housekeeping genes used for normalization of the PAPD5 and PAPD7 mRNA measured with the primer assay indicated below the housekeeping gene.

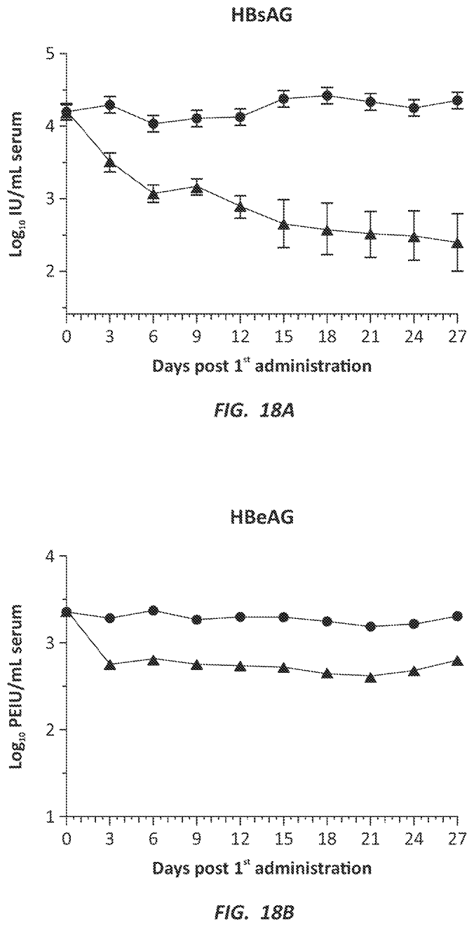

The results are shown in the table 34, 35 and 36 below. The data in table 34 are furthermore presented in FIGS. 18 A and B.

TABLE 34
HBsAg (Log10 IU/mL serum) in AAV/HBV mice treated
with PAPD5 and PAPD7 targeting oligonucleotides

| | | | | PAPD5 and PAPD7 | | |
|---|---|---|---|---|---|---|
| | Control | | | oligonucleotide | | |
| | (5 ml/kg saline) | | | (10 mg/kg each) | | |
| | HBsAg Mean | | No of | HBsAg Mean | | No of |
| Day | (Log10 IU/mL) | SD | animals | (Log10 IU/mL) | SD | animals |
| 0 | 4.21 | 0.19 | 10 | 4.23 | 0.31 | 11 |
| 3 | 4.30 | 0.19 | 10 | 3.50 | 0.43 | 11 |
| 6 | 4.05 | 0.29 | 7 | 3.08 | 0.36 | 8 |
| 9 | 4.12 | 0.29 | 7 | 3.17 | 0.35 | 8 |
| 12 | 4.15 | 0.32 | 7 | 2.89 | 0.44 | 8 |
| 15 | 4.39 | 0.12 | 4 | 2.67 | 0.75 | 5 |
| 18 | 4.45 | 0.23 | 4 | 2.59 | 0.80 | 5 |
| 21 | 4.36 | 0.14 | 4 | 2.51 | 0.73 | 5 |
| 24 | 4.27 | 0.11 | 4 | 2.50 | 0.77 | 5 |
| 27 | 4.37 | 0.06 | 4 | 2.41 | 0.90 | 5 |

The data show that targeting PAPD5 and PAPD7 in the AAV/HBV mouse model with a single treatment resulted in a sustained 2 log reduction in HBsAg up to 27 days after treatment.

TABLE 35
HBeAg (Log10 IU/mL serum) in AAV/HBV mice treated
with PAPD5 and PAPD7 targeting oligonucleotides

| | | | | PAPD5 and PAPD7 | | |
|---|---|---|---|---|---|---|
| | Control | | | oligonucleotide | | |
| | (5 ml/kg saline) | | | (10 mg/kg each) | | |
| | HBeAg Mean | | No of | HBeAg Mean | | No of |
| Day | (Log10 IU/mL) | SD | animals | (Log10 IU/mL) | SD | animals |
| 0 | 3.39 | 0.06 | 10 | 3.40 | 0.05 | 11 |
| 3 | 3.31 | 0.06 | 10 | 2.75 | 0.07 | 11 |
| 6 | 3.39 | 0.05 | 7 | 2.83 | 0.03 | 8 |
| 9 | 3.29 | 0.05 | 7 | 2.77 | 0.04 | 8 |
| 12 | 3.33 | 0.03 | 7 | 2.75 | 0.05 | 8 |
| 15 | 3.32 | 0.06 | 4 | 2.74 | 0.05 | 5 |
| 18 | 3.28 | 0.04 | 4 | 2.67 | 0.02 | 5 |
| 21 | 3.22 | 0.03 | 4 | 2.63 | 0.01 | 5 |
| 24 | 3.24 | 0.04 | 4 | 2.70 | 0.03 | 5 |
| 27 | 3.32 | 0.05 | 4 | 2.80 | 0.04 | 5 |

As for HBsAg the targeting of PAPD5 and PAPD7 leads to reduction in HBeAg levels in the serum, although not as significant as for HBsAg.

TABLE 36
PAPD5 and PAPD7 mRNA in AAV/HBV mice (3 animals on day 3 and
14 and 5 on day 27) and ALT levels (11 animals day 0, 8 on day
14 and 5 on day 27) following a single dose treatment with PAPD5
and PAPD7 targeting oligonucleotides (10 mg/kg of each).

| | % PAPD5 | | % PAPD7 | | | | | |
|---|---|---|---|---|---|---|---|---|
| | mRNA | | mRNA | | ALT (U/L) | | | |
| | of control | | of control | | Control | | Treated | |
| Day | Avg | sd | Avg | sd | Avg | sd | Avg | sd |
| 0 | NA | NA | NA | NA | 30.91 | 6.95 | 27.27 | 7.55 |
| 3 | 14.47 | 4.20 | 24.82 | 3.43 | NA | NA | NA | NA |
| 14 | 21.995 | 5.13 | 20.37 | 1.75 | 37.50 | 14.49 | 47.00 | 26.51 |
| 27 | 37.543 | 7.65 | 27.52 | 8.08 | 28.80 | 9.55 | 28.00 | 18.97 |

From these data it can be seen that the PAPD5 and PAPD7 targeting oligonucleotides leads to reduction in PAPD5 and PAPD7 mRNA levels, respectively, and are well tolerated in the AAV/HBV mouse model.

1. An antisense oligonucleotide of 12 to 30 nucleotides in length, which comprises a contiguous nucleotide sequence of 12 to 20 nucleotides in length which is capable of inhibiting the expression of both PAPD5 and PAPD7.

2. The nucleic acid molecule of claim 1, wherein the contiguous nucleotide sequence is at least 93% complementarity to the target nucleic acid of SEQ ID NO: 1 and SEQ ID NO: 2.

3. The nucleic acid molecule of claim 1 or 2, wherein the contiguous nucleotide sequence is complementary to position 64669 to 69429 on SEQ ID NO: 1 and position 29514 to 29530 on SEQ ID NO: 2.

4. The nucleic acid molecule of claim 1 or 2, wherein the contiguous nucleotide sequence is complementary to position 69414 to 69429 on SEQ ID NO: 11 and position 30731 to 30746 on SEQ ID NO: 2.

5. The antisense oligonucleotide of claims 1 to 3, wherein the contiguous nucleotide sequence comprises or consists of SEQ ID NO: 17 or 18

6. The antisense oligonucleotide of any one of claims 1 to 5, comprising one or more 2′ sugar modified nucleoside(s).

7. The antisense oligonucleotide of claim 6, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

8. The antisense oligonucleotide of claim 6 or 7, wherein the one or more 2′ sugar modified nucleoside is a LNA nucleoside.

9. The antisense oligonucleotide of claim 7 or 8, wherein the LNA nucleoside is oxy-LNA, or cET.

10. The antisense oligonucleotide of any one of claims 1 to 9, wherein at least 75% of the internucleoside linkages within the contiguous nucleotide sequence are phosphorothioate internucleoside linkages.

11. The antisense oligonucleotide of 10, wherein at least one of the phosphorothioate internucleoside linkages are stereodefined.

12. The antisense oligonucleotide of any one of claims 1 to 10, wherein the oligonucleotide is a gapmer of formula 5′-F-G-F′-3′, where the F and F′ wing regions independently comprise 1-8 2′ sugar modified nucleosides in accordance with claims 7 to 10 and G is a gap region between 5 and 16 nucleosides which are capable of recruiting RNaseH.

13. The antisense oligonucleotide of claim 12, wherein
a. the F region is between 1 and 6 nucleotides in length and consists of 1-5 identical LNA nucleosides and 0-3 DNA nucleosides; and
b. the F′ region is between 2 and 6 nucleotides in length and consists of 2-5 identical LNA nucleosides and 0-3 DNA nucleosides; and
c. the G region consists of between 5 and 11 DNA nucleotides.

14. The antisense oligonucleotide of any one of claims 1 to 13, wherein the antisense oligonucleotide is selected from the group consisting of CMP ID NO: 18_1, 18_5, 18_10, 18_15, 18_18, 18_19, 18_24, 18_27, 18_30, 18_346, 18_347, 18_357, 17_10, 17_137 and 17_139.

15. A conjugate compound comprising the antisense oligonucleotide according to any one of claims 1 to 14 and at least one conjugate moiety covalently attached to said oligonucleotide.

16. The conjugate compound of claim 15, wherein the conjugate moiety is selected from one of the trivalent GalNAc moieties in FIG. 1.

17. The conjugate compound of claim 15 or 16, wherein the conjugate moiety is the trivalent GalNAc moiety in FIG. 1D.

18. The conjugate compound of any one of claims 15-17, comprising a linker positioned between the antisense oligonucleotide and the conjugate moiety.

19. The conjugate compound of claim 18, wherein the linker is a physiologically labile linker composed of 2 to 5 consecutive phosphodiester linked nucleosides at the 5′ or 3′ terminal of the contiguous nucleotide sequence of the antisense compound.

20. The conjugate compound of any one of claims 15 to 19, wherein the conjugate compound is selected from the group consisting of CMP ID NO: 20_12, 20_13, 20_14, 20_15, 20_16, 20_18, 20_20, 20_21, 2022, 20_30, 20_35, 20_36, 21_2, 21_33 and 2134.

21. A pharmaceutical composition comprising the antisense oligonucleotide according to any one of claims 1 to 14, or the conjugate compound of any one of claims 15-19, or acceptable salts thereof and a pharmaceutically acceptable diluent, carrier, salt and/or adjuvant.

22. An in vivo or in vitro method for modulating PAPD5 and PAPD7 expression in a target cell which is expressing PAPD5 and PAPD7, said method comprising administering an antisense oligonucleotide according to any one of claims 1 to 14, or the conjugate compound of any one of claims 15-19 in an effective amount to said cell.

23. The antisense oligonucleotide according to any one of claims 1 to 14, or the conjugate compound of any one of claims 15-19 or the pharmaceutical composition of claim 21 for use as a medicament.

24. The antisense oligonucleotide according to any one of claims 1 to 14, or the conjugate compound of any one of claims 15-19 or the pharmaceutical composition of claim 21, for use in the treatment or prevention of HBV infection or chronic HBV infection or reduction of the infectiousness of a HBV infected person.
